(19) United States
(12) Patent Application Publication
(10) Pub. No.: US 2022/0388964 A1
(43) Pub. Date: Dec. 8, 2022

(54) (AZA)INDAZOLYL-ARYL SULFONAMIDE AND RELATED COMPOUNDS AND THEIR USE IN TREATING MEDICAL CONDITIONS

(71) Applicants: HiberCell, Inc., New York, NY (US); Lycera Corporation, Ann Arbor, MI (US)

(72) Inventors: Savithri Ramurthy, New York, NY (US); Mark J. Mulvihill, New York, NY (US); Bradley Sherborne, New York, NY (US); Eric Talbot, New York, NY (US); Chris Thomson, New York, NY (US); Thomas Daniel Aicher, Ann Arbor, MI (US); Fernando Padilla, Ann Arbor, MI (US); Clarke B. Taylor, Ann Arbor, MI (US); Peter L. Toogood, Ann Arbor, MI (US)

(21) Appl. No.: 17/603,267

(22) PCT Filed: Apr. 13, 2020
(86) PCT No.: PCT/US2020/027991

§ 371 Date: Oct. 12, 2021

Related U.S. Application Data
(60) Provisional application No. 62/832,982, filed on Apr. 12, 2019.

(51) Int. Cl. C07D 231/56; C07D 401/04; C07D 403/04; C07D 471/04; C07D 401/14; C07D 403/14; C07D 413/14; C07D 401/12; C07D 405/14; C07D 417/14; C07D 403/12

(52) U.S. Cl. CPC C07D 231/56; C07D 401/04; C07D 403/04; C07D 471/04; C07D 401/14; C07D 403/14; C07D 413/14; C07D 401/12; C07D 405/14; C07D 417/14; C07D 403/12

(57) ABSTRACT

The invention provides (aza)indazolyl-aryl sulfonamide and related compounds, pharmaceutical compositions, and their use in the treatment of medical conditions, such as cancer, and in inhibiting GCN2 activity.

### CROSS REFERENCE TO RELATED APPLICATIONS

This application is a U.S. national stage application under 35 U.S.C. § 371 of International Application No. PCT/US2020/027991, filed Apr. 13, 2020, which claims priority to and the benefit of U.S. Provisional Application No. 62/832,982, filed on Apr. 12, 2019, the entire contents of each of which are incorporated herein by reference in their entirety.

### FIELD OF THE INVENTION

The invention provides (aza)indazolyl-aryl sulfonamide and related compounds, pharmaceutical compositions, and their use in the treatment of medical conditions, such as cancer, and in inhibiting GCN2 activity.

### BACKGROUND

Cancer continues to be a significant health problem despite the substantial research efforts and scientific advances reported in the literature for treating this disease. Some of the most frequently diagnosed cancers include prostate cancer, breast cancer, and lung cancer. Prostate cancer is the most common form of cancer in men. Breast cancer remains a leading cause of death in women. Current treatment options for these cancers are not effective for all patients and/or can have substantial adverse side effects. New therapies are needed to address this unmet need in cancer therapy.

General control nonderepressible kinase 2 (GCN2) is a serine/threonine protein kinase that phosphorylates the a subunit of eukaryotic initiation factor 2 (eIF2α) in response to amino acid deficiency (see, for example, Wek, R. C. et al. in Biochem. Soc. Trans. 2006, 34(Pt 1), p. 7-11). Expression and activation of GCN2 have been shown to be elevated in human and mouse tumors, and reduction in the expression of GCN2 has been shown to inhibit tumor growth (see, e.g., Ye, J. et al. in EMBO J. 2010, 29(12), p. 2082-2096). Tumors grow in an environment of amino acid deficiency which can be further depleted with chemotherapy inducing a dependence on autophagy which requires GCN2 activity. In addition, GCN2 mediates the induction of anergy in T cells in response to tryptophan depletion by indoleamine 2,3-dioxygenase (IDO) in the tumor microenvironment (Munn, D. H. et al in Immunity 2005, 22, p. 633-642) and is essential for the proliferative fitness of cytotoxic T cells in amino acid limiting environments (Van de Velde, L-A., et al. in Cell Reports 2016, 17, p. 2247-2258). Inhibition of GCN2 has been reported as a therapeutic approach for cancer therapy (see, e.g., Wei, C. et al. in Mol. Biol. Cell. 2015, 26(6), p. 1044-1057). Accordingly, compounds having inhibitory activity towards GCN2 are needed as therapeutic agents for treating cancer.

The present invention addresses this need and provides other related advantages.

### SUMMARY

The invention provides (aza)indazolyl-aryl sulfonamide and related compounds, pharmaceutical compositions, and their use in the treatment of medical conditions, such as cancer, and in inhibiting GCN2 activity. In particular, one aspect of the invention provides a collection of (aza)indazolyl-aryl sulfonamide and related compounds, such as a compound represented by Formula I:

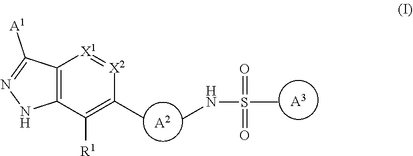

or a pharmaceutically acceptable salt thereof, where the variables are as defined in the detailed description. Another aspect of the invention provides a collection of (aza)indazolyl-aryl sulfonamide and related compounds represented by Formula II:

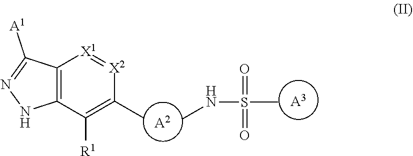

or a pharmaceutically acceptable salt thereof, where the variables are as defined in the detailed description. Further description of additional collections of (aza)indazolyl-aryl sulfonamide and related compounds are described in the detailed description. The compounds may be part of a pharmaceutical composition comprising a pharmaceutically acceptable carrier.

Another aspect of the invention provides a method of treating cancer in a subject. The method comprises administering a therapeutically effective amount of a compound described herein, such as a compound of Formula I, I-1, I-A, I-B, II, or II-A, to a subject in need thereof to treat the cancer. In certain embodiments, the cancer is a solid tumor, leukemia, or lymphoma. In certain other embodiments, the cancer is colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, sweat gland carcinoma, sebaceous gland carcinoma, thyroid cancer, kidney cancer, uterus cancer, esophagus cancer, liver cancer, head cancer, neck cancer, throat cancer, mouth cancer, bone cancer, chest cancer, lymph node cancer, eye cancer, mesothelioma, an acoustic neuroma, oligodendroglioma, meningioma, neuroblastoma, retinoblastoma, leukemia, or lymphoma. The compound may be used as monotherapy or as part of a combination therapy, to treat the cancer.

Another aspect of the invention provides a method of treating a neurodegenerative disease in a subject. The method comprises administering a therapeutically effective amount of a compound described herein, such as a compound of Formula I, I-1, I-A, I-B, II, or II-A, to a subject in need thereof to treat the neurodegenerative disease. In certain embodiments, the neurodegenerative disease is Alzheimer's disease, Parkinson's Disease, Huntington's Disease, amyotrophic lateral sclerosis, or spinocerebellar ataxia.

Another aspect of the invention provides a method of treating doxorubicin-induced cardiotoxicity in a subject. The method comprises administering a therapeutically effective amount of a compound described herein, such as a compound of Formula I, I-1, I-A, I-B, II, or II-A, to a subject in need thereof suffering from doxorubicin-induced cardiotoxicity, to thereby treat the doxorubicin-induced cardiotoxicity.

Another aspect of the invention provides a method of inhibiting the activity of GCN2. The method comprises exposing a GCN2 to an effective amount of a compound described herein, such as a compound of Formula I, I-1, I-A, I-B, II, or II-A, to inhibit the activity of said GCN2.

### DETAILED DESCRIPTION

The invention provides (aza)indazolyl-aryl sulfonamide and related compounds, pharmaceutical compositions, and their use in the treatment of medical conditions, such as cancer, and in inhibiting GCN2 activity. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name, and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “—O-alkyl” etc.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkylene” refers to a diradical of an alkyl group. Exemplary alkylene groups include —CH₂—, —CH₂CH₂—, and —CH₂C(H)(CH₃)CH₂—. The term “—(C₀ alkylene)-” refers to a bond. Accordingly, the term “—(C₀₋₃ alkylene)-” encompasses a bond (i.e., C₀) and a —(C₁₋₃ alkylene) group.

As used herein, “carbocyclyl” or “carbocyclic” refers to a radical of a non-aromatic cyclic hydrocarbon group having from 3 to 10 ring carbon atoms (“C3-10 carbocyclyl”) and zero heteroatoms in the non-aromatic ring system. In certain embodiments, a carbocyclyl group has 3 to 8 ring carbon atoms. In certain embodiments, a carbocyclyl group has 3 to 7 ring carbon atoms (“C3-7 carbocycyl”). In certain embodiments, a carbocyclyl group has 3 to 6 ring carbon atoms (“C3-6 carbocyclyl”). In certain embodiments, a carbocyclyl group has 5 to 10 ring carbon atoms (“C5-10 carbocyclyl”). In certain embodiments, a carbocyclyl group has 5 to 10 ring carbon atoms (“C7-10 carbocyclyl”). Exemplary C3-6 carbocyclyl groups include, without limitation, cyclopropyl (C3), cyclobutyl (C4), cyclobutenyl (C4), cyclopentyl (C5), cyclopentenyl (C5), cyclohexyl (C6), cyclohexenyl (C6), cyclohexadienyl (C6), and the like. Exemplary C3-8 carbocyclyl groups include, without limitation, the aforementioned C3-6 carbocyclyl groups as well as cycloheptyl (C7), cycloheptenyl (C7), cycloheptadienyl (C7), cycloheptatrienyl (C7), cyclooctyl (C8), cyclooctenyl (C8), bicyclo[2.2.1]heptanyl (C7), bicyclo[2.2.2]octanyl (C8), and the like. Exemplary C3-10 carbocyclyl groups include, without limitation, the aforementioned C3-8 carbocyclyl groups as well as cyclononyl (C9), cyclononenyl (C9), cyclodecyl (C10), cyclodecenyl (C10), octahydro-1H-indenyl (C9), decahydronaphthalenyl (C10), spiro[4.5]decanyl (C10), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or partially unsaturated.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “halocycloalkyl” refers to a cycloalkyl group that is substituted with at least one halogen.

The term “cycloalkylene” refers to a diradical of a cycloalkyl group. Exemplary cycloalkylene groups include

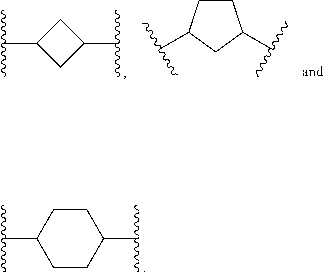

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The term “hydroxyfluoroalkyl” refers to a hydroxyalkyl that is substituted with at least one fluoro.

The term “aralkyl” refers to an alkyl group substituted with an aryl group. Exemplary aralkyl groups include

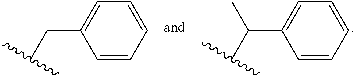

The term “heteroaralkyl” refers to an alkyl group substituted with a heteroaryl group.

The terms “alkenyl” and “alkynyl” are art-recognized and refer to unsaturated aliphatic groups analogous in length and possible substitution to the alkyls described above, but that contain at least one double or triple bond respectively.

The term “cycloalkenyl” refers to a monovalent unsaturated cyclic, bicyclic, or bridged (e.g., adamantyl) carbocyclic hydrocarbon containing at least one C—C double bond. In certain embodiments, the cycloalkenyl contains 5-10, 5-8, or 5-6 carbons, referred to herein, e.g., as “C₅-C₆ cycloalkenyl”. Exemplary cycloalkenyl groups include cyclohexenyl and cyclopentenyl.

The term “aryl” is art-recognized and refers to a carbocyclic aromatic group. Representative aryl groups include phenyl, naphthyl, anthracenyl, and the like. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “aryl” also includes polycyclic aromatic ring systems having two or more carbocyclic rings in which two or more carbons are common to two adjoining rings (the rings are “fused rings”) wherein all of the fused rings are aromatic rings, e.g., in a naphthyl group.

The term “phenylene” refers to a diradical of a phenyl group. Exemplary phenylene groups include

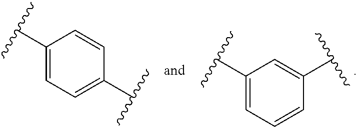

The term “heteroaryl” is art-recognized and refers to aromatic groups that include at least one ring heteroatom. In certain instances, a heteroaryl group contains 1, 2, 3, or 4 ring heteroatoms (e.g., O, N, and S). Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic aromatic ring systems having two or more rings in which two or more ring atoms are common to two adjoining rings (the rings are “fused rings”) wherein all of the fused rings are heteroaromatic, e.g., in a naphthyridinyl group. In certain embodiments, the heteroaryl is a 5-6 membered monocyclic ring or a 9-10 membered bicyclic ring.

The term “heteroarylene” refers to a diradical of a heteroaryl group. Exemplary heteroarylene groups include: phenylene, pyridinylene, pyridazinylene, pyrimidinylene, pyrazinylene,

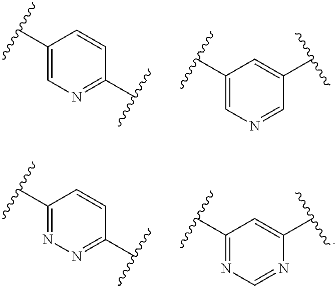

The terms ortho, meta, and para are art-recognized and refer to 1,2-, 1,3- and 1,4-disubstituted benzenes, respectively. For example, the names 1,2-dimethylbenzene and ortho-dimethylbenzene are synonymous.

As used herein, the terms “heterocyclic” and “heterocyclyl” represent, for example, an aromatic or nonaromatic ring (e.g., a saturated, partially saturated, or unsaturated monocyclic or bicyclic ring) containing one or more ring heteroatoms. The heteroatoms can be the same or different from each other. Examples of heteroatoms include, but are not limited to nitrogen, oxygen and sulfur. In certain instances, a heterocyclic group contains 1, 2, 3, or 4 ring heteroatoms (e.g., O, N, and S). Aromatic and nonaromatic heterocyclic rings are well-known in the art. Some nonlimiting examples of aromatic heterocyclic rings include, but are not limited to, pyridine, pyrimidine, indole, purine, quinoline and isoquinoline. Nonlimiting examples of nonaromatic heterocyclic compounds include, but are not limited to, piperidine, piperazine, morpholine, pyrrolidine and pyrazolidine. Examples of oxygen containing heterocyclic rings include, but are not limited to, furan, oxirane, 2H-pyran, 4H-pyran, 2H-chromene, benzofuran, and 2,3-dihydrobenzo[b][1,4]dioxine. Examples of sulfur-containing heterocyclic rings include, but are not limited to, thiophene, benzothiophene, and parathiazine. Examples of nitrogen containing rings include, but are not limited to, pyrrole, pyrrolidine, pyrazole, pyrazolidine, imidazole, imidazoline, imidazolidine, pyridine, piperidine, pyrazine, piperazine, pyrimidine, indole, purine, benzimidazole, quinoline, isoquinoline, triazole, and triazine. Examples of heterocyclic rings containing two different heteroatoms include, but are not limited to, phenothiazine, morpholine, parathiazine, oxazine, oxazole, thiazine, and thiazole. The heterocyclic ring is optionally further substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the heterocyclyl group is a 3-7 membered ring that, unless specified otherwise, is substituted or unsubstituted. In certain embodiments, the heterocyclyl group is a 3-7 membered ring that contains 1, 2, or 3 ring heteroatoms selected from oxygen, sulfur, and nitrogen.

The term “heterocycloalkyl” refers to a saturated heterocyclyl group having, for example, 3-7 ring atoms selected from carbon and heteroatoms (e.g., O, N, or S).

The terms “amine” and “amino” are art-recognized and refer to both unsubstituted and substituted amines, e.g., a moiety that may be represented by the general formulas:

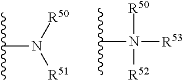

wherein R⁵⁰, R⁵¹, R⁵² and R⁵³ each independently represent a hydrogen, an alkyl, an alkenyl, —(CH₂)ₘ—R⁶¹, or R⁵⁰ and R⁵¹, taken together with the N atom to which they are attached complete a heterocycle having from 4 to 8 atoms in the ring structure; R⁶¹ represents an aryl, a cycloalkyl, a cycloalkenyl, a heterocycle or a polycycle; and m is zero or an integer in the range of 1 to 8. In certain embodiments, only one of R⁵⁰ or R⁵¹ may be a carbonyl, e.g., R⁵⁰, R⁵¹ and the nitrogen together do not form an imide. In other embodiments, R⁵⁰ and R⁵¹ (and optionally R⁵²) each independently represent a hydrogen, an alkyl, an alkenyl, or —(CH₂)ₘ—R⁶¹.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and —O—(CH₂)ₘ—R⁶¹, where m and R⁶¹ are described above.

The term “fluoroalkoxyl” refers to an alkoxyl group that is substituted with at least one fluoro group. Exemplary fluoroalkoxyl groups include —OCH₂F, —OCHF₂, —OCF₃, —OCH₂CF₃, —OCF₂CF₃, and the like.

The term “oxo” is art-recognized and refers to a “═O” substituent. For example, a cyclopentane substituted with an oxo group is cyclopentanone.

The symbols “ [symbol] ”, “*”, and “**” indicate a point of attachment.

The term “substituted” means that one or more hydrogens on the atoms of the designated group are replaced with a selection from the indicated group, provided that the atoms' normal valences under the existing circumstances are not exceeded, and that the substitution results in a stable compound. Combinations of substituents and/or variables are permissible only if such combinations result in stable compounds. The terms “stable compound” or “stable structure” refer to a compound that is sufficiently robust to survive isolation to a useful degree of purity from a reaction mixture, and formulation into an efficacious therapeutic agent.

When any substituent or variable occurs more than one time in any constituent or the compound of the invention, its definition on each occurrence is independent of its definition at every other occurrence, unless otherwise indicated.

It should also be noted that any carbon as well as heteroatom with unsatisfied valences in the text, schemes, examples and tables herein is assumed to have the sufficient number of hydrogen atom(s) to satisfy the valences.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Nonlimiting examples of suitable solvates include ethanolates, methanolates, and the like. “Hydrate” is a solvate wherein the solvent molecule is H₂O.

Certain compounds contained in compositions of the present invention may exist in particular geometric or stereoisomeric forms. Further, certain compounds described herein may be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. The compounds may contain one or more stereogenic centers. For example, asymmetric carbon atoms may be present in a substituent such as an alkyl group. All such isomers, as well as mixtures thereof, are intended to be included in this invention, such as, for example, racemic mixtures, single enantiomers, diastereomeric mixtures and individual diastereomers. Additional asymmetric centers may be present depending upon the nature of the various substituents on the molecule. Each such asymmetric center will independently produce two optical isomers, and it is intended that all of the possible optical isomers, diastereomers in mixtures, and pure or partially purified compounds are included within the ambit of this invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as a atropisomer (e.g., substituted biaryls), all forms of such atropisomer are considered part of this invention.

As used herein, the terms “subject” and “patient” are used interchangeable and refer to organisms to be treated by the methods of the present invention. Such organisms preferably include, but are not limited to, mammals (e.g., murines, simians, equines, bovines, porcines, canines, felines, and the like), and most preferably includes humans.

The term “IC₅₀” is art-recognized and refers to the concentration of a compound that is required to achieve 50% inhibition of the target.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₃, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate (also known as toluenesulfonate), undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. Further examples of salts include, but are not limited to: ascorbate, borate, nitrate, phosphate, salicylate, and sulfate. Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Additional exemplary basic salts include, but are not limited to: ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

The present invention includes the compounds of the invention in all their isolated forms (such as any solvates, hydrates, stereoisomers, and tautomers thereof). Further, the invention includes compounds in which one or more of the atoms may be artificially enriched in a particular isotope having the same atomic number, but an atomic mass or mass number different from the atomic mass or mass number predominantly found in nature. The present invention is meant to include all suitable isotopic variations of the compounds of the invention. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds can be prepared without undue experimentation by conventional techniques known to those skilled in the art or by processes analogous to those described in the Schemes and Examples herein using appropriate isotopically-enriched reagents and/or intermediates.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

The terms “a” and “an” as used herein mean “one or more” and include the plural unless the context is inappropriate.

The abbreviation “THF” is art-recognized and refers to tetrahydrofuran. The abbreviation “DCM” is art-recognized and refers to dichloromethane. The abbreviation “DMF” is art-recognized and refers to dimethylformamide. The abbreviation “DMA” is art-recognized and refers to dimethylacetamide. The abbreviation “EDTA” is art-recognized and refers to ethylenediaminetetraacetic acid. The abbreviation “TFA” is art-recognized and refers to trifluoroacetic acid. The abbreviation “Ts” is art-recognized and refers to tosylate. The abbreviation “TBS” is art-recognized and refers to tert-butyldimethylsilyl. The abbreviation “DMSO” is art-recognized and refers to dimethylsulfoxide. The abbreviation “Tf” is art-recognized and refers to triflate, or trifluoromethylsulfonate. The abbreviation “Pin” is art-recognized and refers to pinacolato.

As a general matter, compositions specifying a percentage are by weight unless otherwise specified.

### I. (Aza)Indazolyl-Aryl Sulfonamide and Related Compounds

The invention provides (aza)indazolyl-aryl sulfonamide and related compounds. The compounds may be used in the pharmaceutical compositions and therapeutic methods described herein. Exemplary compounds are described in the following sections, along with exemplary procedures for making the compounds. Additional exemplary compounds and synthetic procedures are described in the Examples.

One aspect of the invention provides a compound represented by Formula I:

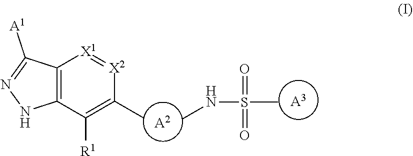

or a pharmaceutically acceptable salt thereof; wherein:

X¹ and X² are independently C(R²) or N, wherein X¹ is N and X² is C(R²), X¹ is C(R²) and X² is N, or both X¹ and X² are C(R²)

R¹ is halogen, hydrogen, C₁₋₄ alkyl, C₁₋₄ fluoroalkyl, or cyano;

R² represents independently for each occurrence hydrogen, halogen, C₁₋₄ alkyl, C₁₋₄ fluoroalkyl, cyano, C₁₋₄ alkoxyl, or hydroxyl;

R³ and R⁴ each represent independently for each occurrence hydrogen, C₁₋₄ alkyl, or C₃₋₇ cycloalkyl; or an occurrence of R³ and R⁴ attached to the same nitrogen atom are taken together with the nitrogen atom to which they are attached to form a 3-7 membered carbocyclyl or heterocyclyl;

R⁵ represents independently for each occurrence hydrogen, C₁₋₄ alkyl, or hydroxyl;

R⁶ represents independently for each occurrence hydrogen, C₁₋₆ alkyl, C₁₋₆ hydroxyalkyl, C₃₋₆ cycloalkyl, 4-7 membered heterocyclyl, 6-10 membered aryl, —(C₁₋₆ alkylene)-N(R³)(R⁴), —(C₁₋₆ alkylene)-N(R³)—C(O)(R⁴), —(C₁₋₆ alkylene)-(5-10 membered heteroaryl), —(C₁₋₆ alkylene)-C₃₋₆ cycloalkyl), —(C₁₋₆ alkylene)-(5-10 membered heterocycloalkyl), —(C₁₋₆ alkylene)-CO₂R³, —(C₁₋₆ alkylene)-C(O)N(R³)(R⁴), —(C₁₋₆ alkylene)-S(O)₂—(C₁₋₆ alkyl), —(C₁₋₆ alkylene)-O—(C₁₋₆ alkyl), or —(C₁₋₆ alkylene)-CN, wherein the C₁₋₆ alkyl, C₃₋₆ cycloalkyl, —(C₁₋₆ alkylene)-(C₃₋₆ cycloalkyl), 4-7 membered heterocyclyl, and —(C₁₋₆ alkylene)-(5-10 membered heterocycloalkyl) may be optionally substituted with 1, 2, or 3 substituents independently selected from the group consisting cyano, halogen, hydroxyl, oxo, and NH₂, and wherein if the 4-7 membered heterocyclyl and —(C₁₋₆ alkylene)-(5-10 membered heterocycloalkyl) contain a suitable ring nitrogen atom, that ring nitrogen may be optionally substituted by C₁₋₃ alkyl or —C(O)—C₁₋₃ alkyl;

R⁷ is C₁₋₄ alkyl, C₃₋₇ cycloalkyl, or —(C₁₋₆ alkylene)-(C₃₋₇ cycloalkyl);

A¹ is one of the following:
5-10 membered heterocyclyl or 6-10 membered aryl, each of which is optionally substituted with 1, 2, or 3 substituents independently selected from the group consisting of halogen, C₁₋₆ alkyl, C₁₋₆ hydroxyalkyl, C₁₋₄ haloalkyl, C₁₋₄ hydroxyfluoroalkyl, C₃₋₅ cycloalkyl, cyano, hydroxyl, C₁₋₄ alkoxyl, oxo, —N(R³)(R⁴), —N(R³)—C(O)(R⁴), —(C₁₋₆ alkylene)-N(R³)(R⁴), —C(O)N(R⁵)(R⁶), and —(C₁₋₆ alkylene)-C(O)N(R⁵)(R⁶); or
—C(O)N(R⁵)(R⁶) or —N(R⁵)C(O)(R⁷);

A² is phenylene or a 5-6 membered heteroarylene, each of which is optionally substituted with 1, 2, or 3 substituents independently selected from the group consisting of halogen, C₁₋₄ alkyl, C₁₋₄ haloalkyl, cyano, C₁₋₄ alkoxyl, C₃₋₅ cycloalkyl, and C₃₋₅ halocycloalkyl; and

A³ is phenyl, —CH₂—(C₃₋₆ cycloalkyl), 7-10 membered bicyclic carbocyclyl, or 5-10 membered heterocyclyl, each of which is optionally substituted with 1, 2, or 3 substituents independently selected from the group consisting of halogen, C₁₋₆ alkyl, C₁₋₆ hydroxyalkyl, C₁₋₄ haloalkyl, C₁₋₄ hydroxyfluoroalkyl, C₃₋₅ cycloalkyl, cyano, hydroxyl, C₁₋₄ alkoxyl, C₁₋₄ fluoroalkoxyl, —N(R³)(R⁴), —N(R³)—C(O)(R⁴), —(C₁₋₆ alkylene)-N(R³)(R⁴), —CO₂H, —CO₂(C₁₋₆ alkyl), —S—(C₁₋₆ alkyl), and —S—(C₁₋₆ fluoroalkyl), wherein each of the 7-10 membered bicyclic carbocyclyl and 5-10 membered heterocyclyl is optionally further substituted by oxo or oxime, and wherein if the 5-10 membered heterocyclyl contains a suitable ring nitrogen atom, that ring nitrogen may be optionally substituted by C₁₋₃ alkyl.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I.

In certain embodiments, X¹ and X² are both C(R²). In certain embodiments, X¹ is C(R²), and X² is N. In certain embodiments, X¹ is N, and X² is C(R²).

In certain embodiments, R¹ is halogen. In certain embodiments, R¹ is fluoro.

In certain embodiments, R² is hydrogen.

In certain embodiments, R³ and R⁴ each represent independently for each occurrence hydrogen or C₁₋₄ alkyl.

In certain embodiments, A¹ is 5-10 membered heterocyclyl or 6-10 membered aryl, each of which is optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, —N(R³)(R⁴), —(C₁₋₆ alkylene)-N(R³)(R⁴), —C(O)N(R⁵)(R⁶), and —(C₁₋₆ alkylene)-C(O)N(R⁵)(R⁶). In certain embodiments, A¹ is 5-10 membered heterocyclyl or 6-10 membered aryl, each of which is optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, —N(R³)(R⁴), and —(C₁₋₆ alkylene)-N(R³)(R⁴). In certain embodiments, A¹ is 5-10 membered heterocyclyl optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, —N(R³)(R⁴), and —(C₁₋₆ alkylene)-N(R³)(R⁴). In certain embodiments, A¹ is a 5-10 membered unsaturated heterocyclyl optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, —N(R³)(R⁴), and —(C₁₋₆ alkylene)-N(R³)(R⁴).

In certain embodiments, A¹ is a 5-membered heteroaryl optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, —N(R³)(R⁴), and —(C₁₋₆ alkylene)-N(R³)(R⁴). In certain embodiments, A¹ is a 5-membered heteroaryl optionally substituted with 1, 2, or 3 substituents independently selected from the group consisting of halogen, C₁₋₆ alkyl, C₁₋₆ hydroxyalkyl, and C₁₋₄ alkoxyl.

In certain embodiments, A¹ is 5-10 membered unsaturated heterocyclyl containing a ring —N(H)— group at the 2-position in A¹, wherein said heterocyclyl is optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, —N(R³)(R⁴), and —(C₁₋₆ alkylene)-N(R³)(R⁴).

In certain embodiments, A¹ is 5-10 membered unsaturated heterocyclyl containing a ring —N(H)— group, wherein said heterocyclyl is optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, —N(R³)(R⁴), and —(C₁₋₆ alkylene)-N(R³)(R⁴). In certain embodiments, A¹ is imidazolyl, pyrazolyl, oxazolyl, pyrrolyl, or furanyl, each of which is optionally substituted with 1, 2, or 3 substituents independently selected from the group consisting of halogen, C₁₋₆ alkyl, C₁₋₆ hydroxyalkyl, and C₁₋₄ alkoxyl. In certain embodiments, A¹ is imidazolyl or pyrazolyl, each of which is optionally substituted with 1, 2, or 3 substituents independently selected from the group consisting of halogen, C₁₋₆ alkyl, C₁₋₆ hydroxyalkyl, and C₁₋₄ alkoxyl. In certain embodiments, A¹ is imidazolyl optionally substituted with 1, 2, or 3 substituents independently selected from the group consisting of halogen, C₁₋₆ alkyl, C₁₋₆ hydroxyalkyl, and C₁₋₄ alkoxyl. In certain embodiments, A¹ is

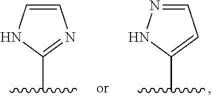 [SMILES: *c1ncc[nH]1 ; *c1ccn[nH]1]

each of which is optionally substituted with one or two substituents independently selected from the group consisting of C₁₋₃ alkyl and C₁₋₆ hydroxyalkyl.

In certain embodiments, A¹ is

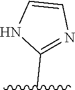 [SMILES: *c1ncc[nH]1]

optionally substituted with one or two C₁₋₃ alkyl.

In certain embodiments, A¹ is —C(O)N(R⁵)(R⁶).

In certain embodiments, R⁵ is hydrogen. In certain embodiments, R⁵ is C₁₋₄ alkyl. In certain embodiments, R⁵ is hydroxyl.

In certain embodiments, R⁶ represents independently for each occurrence hydrogen, C₁₋₆ alkyl, C₁₋₆ hydroxyalkyl, 4-7 membered heterocycloalkyl, phenyl, 5-10 membered heteroaryl, 5-6 membered unsaturated oxo-heterocyclyl, —(C₁₋₆ alkylene)-N(R³)(R⁴), —(C₁₋₆ alkylene)-N(R³)—C(O)(R⁴), —(C₁₋₆ alkylene)-(5-10 membered heteroaryl), —(C₁₋₆ alkylene)-(5-10 membered heterocycloalkyl), —(C₁₋₆ alkylene)-CO₂R³, —(C₁₋₆ alkylene)-C(O)N(R³)(R⁴), —(C₁₋₆ alkylene)-S(O)₂—(C₁₋₆ alkyl), —(C₁₋₆ alkylene)-O—(C₁₋₆ alkyl), or —(C₁₋₆ alkylene)-CN; wherein said heterocycloalkyl, phenyl, heteroaryl, and unsaturated oxo-heterocyclyl are optionally substituted by 1 or 2 substituents independently selected from the group consisting of halogen, C₁₋₄ alkyl, C₁₋₄ fluoroalkyl, C₁₋₄ hydroxyalkyl, cyano, C₁₋₄ alkoxyl, and hydroxyl. In certain embodiments, R⁶ represents independently for each occurrence 4-7 membered heterocycloalkyl, phenyl, 5-10 membered heteroaryl, or 5-6 membered unsaturated oxo-heterocyclyl; wherein said heterocycloalkyl, phenyl, heteroaryl, and unsaturated oxo-heterocyclyl are optionally substituted by 1 or 2 substituents independently selected from the group consisting of halogen, C₁₋₄ alkyl, C₁₋₄ fluoroalkyl, C₁₋₄ hydroxyalkyl, cyano, C₁₋₄ alkoxyl, and hydroxyl. In certain embodiments, R⁶ is hydrogen. In certain embodiments, R⁶ represents independently for each occurrence C₁₋₆ alkyl or C₁₋₆ hydroxyalkyl.

In certain embodiments, A¹ is —N(R⁵)C(O)(R⁷).

In certain embodiments, R⁷ is C₁₋₄ alkyl.

In certain embodiments, A² is phenylene or a 5-6 membered heteroarylene, each of which is optionally substituted with 1, 2, or 3 substituents independently selected from the group consisting of halogen, C₁₋₄ alkyl, C₁₋₄ haloalkyl, and cyano. In certain embodiments, A² is phenylene, pyridinylene, pyridazinylene, pyrimidinylene, or pyrazinylene, each of which is optionally substituted with 1, 2, or 3 substituents independently selected from the group consisting of halogen, C₁₋₄ alkyl, C₁₋₄ haloalkyl, and cyano.

In certain embodiments, A² is phenylene or a 5-6 membered heteroarylene, each of which is optionally substituted with 1, 2, or 3 substituents independently selected from the group consisting of halogen, C₁₋₄ alkyl, C₁₋₄ haloalkyl, cyano, and C₁₋₄ alkoxyl. In certain embodiments, A² is phenylene, pyridinylene, pyridazinylene, pyrimidinylene, or pyrazinylene, each of which is optionally substituted with 1, 2, or 3 substituents independently selected from the group consisting of halogen, C₁₋₄ alkyl, C₁₋₄ haloalkyl, cyano, and C₁₋₄ alkoxyl. In certain embodiments, A² is phenylene optionally substituted with 1, 2, or 3 substituents independently selected from the group consisting of fluoro, chloro, C₁₋₄ alkyl, C₁₋₄ haloalkyl, cyano, and C₁₋₄ alkoxyl. In certain embodiments, A² is pyridinylene optionally substituted with 1, 2, or 3 substituents independently selected from the group consisting of fluoro, chloro, C₁₋₄ alkyl, C₁₋₄ haloalkyl, cyano, and C₁₋₄ alkoxyl.

In certain embodiments, A² is phenylene optionally substituted with 1, 2, or 3 substituents independently selected from the group consisting of fluoro, chloro, C₁₋₄ alkyl, C₁₋₄ haloalkyl, and cyano. In certain embodiments, A² is pyridinylene, pyridazinylene, pyrimidinylene, or pyrazinylene, each of which is optionally substituted with 1, 2, or 3 substituents independently selected from the group consisting of halogen, C₁₋₄ alkyl, C₁₋₄ haloalkyl, and cyano. In certain embodiments, A² is pyridinylene optionally substituted with 1, 2, or 3 substituents independently selected from the group consisting of fluoro, chloro, C₁₋₄ alkyl, C₁₋₄ haloalkyl, and cyano.

In certain embodiments, A² is

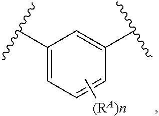

wherein R^{A} is fluoro; n is 1 or 2. In certain embodiments, A² is

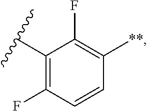

wherein ** is a bond to the sulfonamide nitrogen atom in Formula I.

In certain embodiments, wherein A² is

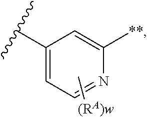

wherein R^{A} is fluoro; w is 1 or 2, and ** is a bond to the sulfonamide nitrogen atom in Formula I.

In certain embodiments, A² is

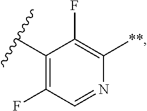

wherein ** is a bond to the sulfonamide nitrogen atom in Formula I.

In certain embodiments, A³ is phenyl substituted with 1, 2, or 3 substituents independently selected from the group consisting of halogen, C₁₋₆ alkyl, C₁₋₆ hydroxyalkyl, C₁₋₄ haloalkyl, cyano, and C₁₋₄ alkoxyl. In certain embodiments, A³ is phenyl substituted with 1, 2, or 3 substituents independently selected from the group consisting of halogen, C₁₋₆ hydroxyalkyl, C₁₋₄ haloalkyl, cyano, and C₁₋₄ alkoxyl. In certain embodiments, A³ is a 5-10 membered unsaturated heterocyclyl optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, —N(R³)(R⁴), and —(C₁₋₆ alkylene)-N(R³)(R⁴).

In certain embodiments, A³ is a 5-6 membered unsaturated heterocyclyl optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, —N(R³)(R⁴), and —(C₁₋₆ alkylene)-N(R³)(R⁴). In certain embodiments, A³ is a 5-6 membered unsaturated heterocyclyl optionally substituted with 1, 2, or 3 substituents independently selected from the group consisting of halogen, C₁₋₆ alkyl, C₁₋₆ hydroxyalkyl, C₁₋₄ alkoxyl, and oxo.

In certain embodiments, A³ is a 5-6 membered heteroaryl optionally substituted with 1, 2, or 3 substituents independently selected from the group consisting of halogen, C₁₋₆ alkyl, C₁₋₆ hydroxyalkyl, and C₁₋₄ alkoxyl. In certain embodiments, A³ is pyridinyl, pyridazinyl, pyrimidinyl, pyrazinyl, pyrazolyl, imidazolyl, or oxazolyl, each of which is optionally substituted with 1, 2, or 3 substituents independently selected from the group consisting of halogen, C₁₋₆ alkyl, C₁₋₆ hydroxyalkyl, and C₁₋₄ alkoxyl.

In certain embodiments, A³ is pyridinyl substituted with 1, 2, or 3 substituents independently selected from the group consisting of halogen, C₁₋₆ alkyl, C₁₋₆ hydroxyalkyl, and C₁₋₄ alkoxyl. In certain embodiments, A³ is pyridinyl substituted with (i) halogen and (ii) C₁₋₆ alkyl or C₁₋₄ alkoxyl. In certain embodiments, A³ is pyridinyl substituted with (i) halogen and (ii) C₁₋₄ alkoxyl. In certain embodiments, A³ is one of the following:

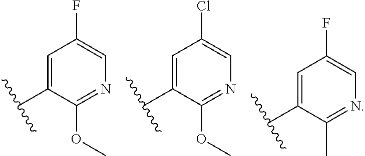

The description above describes multiple embodiments relating to compounds of Formula I. The patent application specifically contemplates all combinations of the embodiments.

Another aspect of the invention provides a compound represented by Formula I-1:

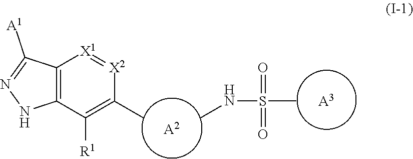

or a pharmaceutically acceptable salt thereof; wherein:

X¹ and X² are independently C(R²) or N, wherein X¹ is N and X² is C(R²), X¹ is C(R²) and X² is N, or both X¹ and X² are C(R²)

R¹ is halogen, hydrogen, C₁₋₄ alkyl, C₁₋₄ fluoroalkyl, or cyano;

R² represents independently for each occurrence hydrogen, halogen, C₁₋₄ alkyl, C₁₋₄ fluoroalkyl, cyano, C₁₋₄ alkoxyl, or hydroxyl;

R³ and R⁴ each represent independently for each occurrence hydrogen, C₁₋₄ alkyl, or C₃₋₇ cycloalkyl; or an occurrence of R³ and R⁴ attached to the same nitrogen atom are taken together with the nitrogen atom to which they are attached to form a 3-7 membered carbocyclyl or heterocyclyl;

R⁵ represents independently for each occurrence hydrogen, C₁₋₄ alkyl, or hydroxyl;

R⁶ represents independently for each occurrence hydrogen, C₁₋₆ alkyl, C₁₋₆ hydroxyalkyl, 4-7 membered heterocycloalkyl, —(C₁₋₆ alkylene)-N(R³)(R⁴), —(C₁₋₆ alkylene)-(5-10 membered heteroaryl), —(C₁₋₆ alkylene)-(5-10 membered heterocycloalkyl), —(C₁₋₆ alkylene)-CO₂R³, —(C₁₋₆ alkylene)-C(O)N(R³)(R⁴), —(C₁₋₆ alkylene)-S(O)₂—(C₁₋₆ alkyl), —(C₁₋₆ alkylene)-O—(C₁₋₆ alkyl), or —(C₁₋₆ alkylene)-CN;

R⁷ is C₁₋₄ alkyl, C₃₋₇ cycloalkyl, or —(C₁₋₆ alkylene)-(C₃₋₇ cycloalkyl);

A¹ is one of the following:
5-10 membered heterocyclyl or 6-10 membered aryl, each of which is optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, —N(R³)(R⁴), —(C₁₋₆ alkylene)-N(R³)(R⁴), —C(O)N(R⁵)(R⁶), and —(C₁₋₆ alkylene)-C(O)N(R⁵)(R⁶); or
—C(O)N(R⁵)(R⁶) or —N(R⁵)C(O)(R⁷);

A² is phenylene or a 5-6 membered heteroarylene, each of which is optionally substituted with 1, 2, or 3 substituents independently selected from the group consisting of halogen, C₁₋₄ alkyl, C₁₋₄ haloalkyl, cyano, C₁₋₄ alkoxyl, C₃₋₅ cycloalkyl, and C₃₋₅ halocycloalkyl; and

A³ is phenyl or a 5-10 membered unsaturated heterocyclyl, each of which is optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, C₁₋₄ fluoroalkoxyl, —N(R³)(R⁴), —(C₁₋₆ alkylene)-N(R³)(R⁴), —CO₂H, —CO₂(C₁₋₆ alkyl), —S—(C₁₋₆ alkyl), and —S—(C₁₋₆ fluoroalkyl), wherein the 5-10 membered unsaturated heterocyclyl is optionally further substituted by oxo.

The definitions of variables in Formula I-1 above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-1.

In certain embodiments, X¹ and X² are both C(R²). In certain embodiments, X¹ is C(R²), and X² is N. In certain embodiments, X¹ is N, and X² is C(R²).

In certain embodiments, R¹ is halogen. In certain embodiments, R¹ is fluoro.

In certain embodiments, R² is hydrogen.

In certain embodiments, R³ and R⁴ each represent independently for each occurrence hydrogen or C₁₋₄ alkyl.

In certain embodiments, A¹ is 5-10 membered heterocyclyl or 6-10 membered aryl, each of which is optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, —N(R³)(R⁴), —(C₁₋₆ alkylene)-N(R³)(R⁴), —C(O)N(R⁵)(R⁶), and —(C₁₋₆ alkylene)-C(O)N(R⁵)(R⁶). In certain embodiments, A¹ is 5-10 membered heterocyclyl or 6-10 membered aryl, each of which is optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, —N(R³)(R⁴), and —(C₁₋₆ alkylene)-N(R³)(R⁴). In certain embodiments, A¹ is 5-10 membered heterocyclyl optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, —N(R³)(R⁴), and —(C₁₋₆ alkylene)-N(R³)(R⁴). In certain embodiments, A¹ is a 5-10 membered unsaturated heterocyclyl optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, —N(R³)(R⁴), and —(C₁₋₆ alkylene)-N(R³)(R⁴).

In certain embodiments, A¹ is a 5-membered heteroaryl optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, —N(R³)(R⁴), and —(C₁₋₆ alkylene)-N(R³)(R⁴). In certain embodiments, A¹ is a 5-membered heteroaryl optionally substituted with 1, 2, or 3 substituents independently selected from the group consisting of halogen, C₁₋₆ alkyl, C₁₋₆ hydroxyalkyl, and C₁₋₄ alkoxyl.

In certain embodiments, A¹ is 5-10 membered unsaturated heterocyclyl containing a ring —N(H)— group at the 2-position in A¹, wherein said heterocyclyl is optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, —N(R³)(R⁴), and —(C₁₋₆ alkylene)-N(R³)(R⁴).

In certain embodiments, A¹ is 5-10 membered unsaturated heterocyclyl containing a ring —N(H)— group, wherein said heterocyclyl is optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, —N(R³)(R⁴), and —(C₁₋₆ alkylene)-N(R³)(R⁴). In certain embodiments, A¹ is imidazolyl, pyrazolyl, oxazolyl, pyrrolyl, or furanyl, each of which is optionally substituted with 1, 2, or 3 substituents independently selected from the group consisting of halogen, C₁₋₆ alkyl, C₁₋₆ hydroxyalkyl, and C₁₋₄ alkoxyl. In certain embodiments, A¹ is imidazolyl or pyrazolyl, each of which is optionally substituted with 1, 2, or 3 substituents independently selected from the group consisting of halogen, C₁₋₆ alkyl, C₁₋₆ hydroxyalkyl, and C₁₋₄ alkoxyl. In certain embodiments, A¹ is imidazolyl optionally substituted with 1, 2, or 3 substituents independently selected from the group consisting of halogen, C₁₋₆ alkyl, C₁₋₆ hydroxyalkyl, and C₁₋₄ alkoxyl. In certain embodiments, A¹ is

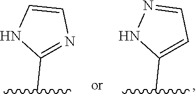 [SMILES: *c1ncc[nH]1 ; *c1ccn[nH]1]

each of which is optionally substituted with one or two substituents independently selected from the group consisting of C₁₋₃ alkyl and C₁₋₆ hydroxyalkyl.

In certain embodiments, A¹ is

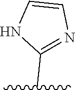 [SMILES: *c1ncc[nH]1]

optionally substituted with one or two C₁₋₃ alkyl.

In certain embodiments, A¹ is —C(O)N(R⁵)(R⁶).

In certain embodiments, R⁵ is hydrogen. In certain embodiments, R⁵ is C₁₋₄ alkyl. In certain embodiments, R⁵ is hydroxyl.

In certain embodiments, R⁶ is hydrogen. In certain embodiments, R⁶ represents independently for each occurrence C₁₋₆ alkyl or C₁₋₆ hydroxyalkyl.

In certain embodiments, A¹ is —N(R⁵)C(O)(R⁷).

In certain embodiments, R⁷ is C₁₋₄ alkyl.

In certain embodiments, A² is phenylene or a 5-6 membered heteroarylene, each of which is optionally substituted with 1, 2, or 3 substituents independently selected from the group consisting of halogen, C₁₋₄ alkyl, C₁₋₄ haloalkyl, and cyano. In certain embodiments, A² is phenylene, pyridinylene, pyridazinylene, pyrimidinylene, or pyrazinylene, each of which is optionally substituted with 1, 2, or 3 substituents independently selected from the group consisting of halogen, C₁₋₄ alkyl, C₁₋₄ haloalkyl, and cyano.

In certain embodiments, A² is phenylene or a 5-6 membered heteroarylene, each of which is optionally substituted with 1, 2, or 3 substituents independently selected from the group consisting of halogen, C₁₋₄ alkyl, C₁₋₄ haloalkyl, cyano, and C₁₋₄ alkoxyl. In certain embodiments, A² is phenylene, pyridinylene, pyridazinylene, pyrimidinylene, or pyrazinylene, each of which is optionally substituted with 1, 2, or 3 substituents independently selected from the group consisting of halogen, C₁₋₄ alkyl, C₁₋₄ haloalkyl, cyano, and C₁₋₄ alkoxyl. In certain embodiments, A² is phenylene optionally substituted with 1, 2, or 3 substituents independently selected from the group consisting of fluoro, chloro, C₁₋₄ alkyl, C₁₋₄ haloalkyl, cyano, and C₁₋₄ alkoxyl. In certain embodiments, A² is pyridinylene optionally substituted with 1, 2, or 3 substituents independently selected from the group consisting of fluoro, chloro, C₁₋₄ alkyl, C₁₋₄ haloalkyl, cyano, and C₁₋₄ alkoxyl.

In certain embodiments, A² is phenylene optionally substituted with 1, 2, or 3 substituents independently selected from the group consisting of fluoro, chloro, C₁₋₄ alkyl, C₁₋₄ haloalkyl, and cyano. In certain embodiments, A² is pyridinylene, pyridazinylene, pyrimidinylene, or pyrazinylene, each of which is optionally substituted with 1, 2, or 3 substituents independently selected from the group consisting of halogen, C₁₋₄ alkyl, C₁₋₄ haloalkyl, and cyano. In certain embodiments, A² is pyridinylene optionally substituted with 1, 2, or 3 substituents independently selected from the group consisting of fluoro, chloro, C₁₋₄ alkyl, C₁₋₄ haloalkyl, and cyano.

In certain embodiments, A² is

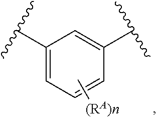

wherein R^{A} is fluoro; n is 1 or 2. In certain embodiments, A² is

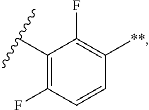

wherein ** is a bond to the sulfonamide nitrogen atom in Formula I-1.

In certain embodiments, A² is

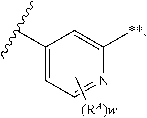

R^{A} is fluoro; w is 1 or 2, and ** is a bond to the sulfonamide nitrogen atom in Formula I-1. In certain embodiments, A² is

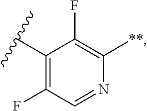

wherein ** is a bond to the sulfonamide nitrogen atom in Formula I-1.

In certain embodiments, A³ is phenyl substituted with 1, 2, or 3 substituents independently selected from the group consisting of halogen, C₁₋₆ alkyl, C₁₋₆ hydroxyalkyl, C₁₋₄ haloalkyl, cyano, and C₁₋₄ alkoxyl. In certain embodiments, A³ is phenyl substituted with 1, 2, or 3 substituents independently selected from the group consisting of halogen, C₁₋₆ hydroxyalkyl, C₁₋₄ haloalkyl, cyano, and C₁₋₄ alkoxyl. In certain embodiments, A³ is a 5-10 membered unsaturated heterocyclyl optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, —N(R³)(R⁴), and —(C₁₋₆ alkylene)-N(R³)(R⁴).

In certain embodiments, A³ is a 5-6 membered unsaturated heterocyclyl optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, —N(R³)(R⁴), and —(C₁₋₆ alkylene)-N(R³)(R⁴). In certain embodiments, A³ is a 5-6 membered unsaturated heterocyclyl optionally substituted with 1, 2, or 3 substituents independently selected from the group consisting of halogen, C₁₋₆ alkyl, C₁₋₆ hydroxyalkyl, C₁₋₄ alkoxyl, and oxo.

In certain embodiments, A³ is a 5-6 membered heteroaryl optionally substituted with 1, 2, or 3 substituents independently selected from the group consisting of halogen, C₁₋₆ alkyl, C₁₋₆ hydroxyalkyl, and C₁₋₄ alkoxyl. In certain embodiments, A³ is pyridinyl, pyridazinyl, pyrimidinyl, pyrazinyl, pyrazolyl, imidazolyl, or oxazolyl, each of which is optionally substituted with 1, 2, or 3 substituents independently selected from the group consisting of halogen, C₁₋₆ alkyl, C₁₋₆ hydroxyalkyl, and C₁₋₄ alkoxyl.

In certain embodiments, A³ is pyridinyl substituted with 1, 2, or 3 substituents independently selected from the group consisting of halogen, C₁₋₆ alkyl, C₁₋₆ hydroxyalkyl, and C₁₋₄ alkoxyl. In certain embodiments, A³ is pyridinyl substituted with (i) halogen and (ii) C₁₋₆ alkyl or C₁₋₄ alkoxyl. In certain embodiments, A³ is pyridinyl substituted with (i) halogen and (ii) C₁₋₄ alkoxyl. In certain embodiments, A³ is one of the following:

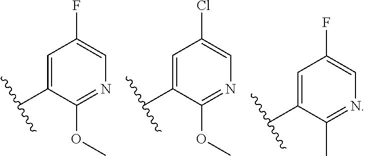

The description above describes multiple embodiments relating to compounds of Formula I-1. The patent application specifically contemplates all combinations of the embodiments.

Another aspect of the invention provides a compound represented by Formula I-A:

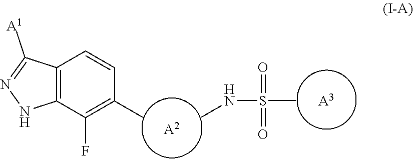

or a pharmaceutically acceptable salt thereof; wherein:

A¹ is one of the following:
5-membered heteroaryl optionally substituted with 1, 2, or 3 substituents independently selected from the group consisting of halogen, C₁₋₆ alkyl, C₁₋₆ hydroxyalkyl, C₁₋₄ haloalkyl, and C₁₋₄ alkoxyl; or
—C(O)N(R⁵)(R⁶), wherein R⁵ and R⁶ each represent independently hydrogen or C₁₋₄ alkyl;

A² is phenylene or pyridinylene, each of which is optionally substituted with 1 or 2 substituents independently selected from the group consisting of fluoro, chloro, C₁₋₂ alkyl, C₁₋₂ haloalkyl, and cyano; and

A³ is phenyl or a 5-6 membered unsaturated heterocyclyl, each of which is optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, and C₁₋₄ alkoxyl.

The definitions of variables in Formula I-A above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-A.

In certain embodiments, A¹ is a 5-membered heteroaryl optionally substituted with 1, 2, or 3 substituents independently selected from the group consisting of halogen, C₁₋₆ alkyl, C₁₋₆ hydroxyalkyl, C₁₋₄ haloalkyl, and C₁₋₄ alkoxyl. In certain embodiments, A¹ is imidazolyl, pyrazolyl, pyrrolyl, furanyl, or oxazolyl, each of which is optionally substituted with 1, 2, or 3 substituents independently selected from the group consisting of halogen, C₁₋₆ alkyl, C₁₋₆ hydroxyalkyl, and C₁₋₄ alkoxyl. In certain embodiments, A¹ is imidazolyl optionally substituted with 1, 2, or 3 substituents independently selected from the group consisting of halogen, C₁₋₆ alkyl, C₁₋₆ hydroxyalkyl, and C₁₋₄ alkoxyl. In certain embodiments, A¹ is

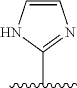 [SMILES: *c1ncc[nH]1]

optionally substituted with one or two C₁₋₃ alkyl.

In certain embodiments, A¹ is —C(O)N(R⁵)(R⁶).

In certain embodiments, A² is phenylene substituted with 1 or 2 substituents independently selected from the group consisting of fluoro and chloro. In certain embodiments, A² is

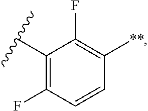

Wherein ** is a bond to the sulfonamide nitrogen atom in Formula I-A. In certain embodiments, A² is

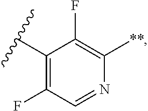

wherein ** is a bond to the sulfonamide nitrogen atom in Formula I-A.

In certain embodiments, A³ is phenyl substituted with 1, 2, or 3 substituents independently selected from the group consisting of halogen, C₁₋₆ hydroxyalkyl, C₁₋₄ haloalkyl, C₃₋₅ cycloalkyl, cyano, hydroxyl, and C₁₋₄ alkoxyl. In certain embodiments, A³ is phenyl substituted with 1, 2, or 3 substituents independently selected from the group consisting of halogen, C₁₋₄ haloalkyl, and C₁₋₄ alkoxyl.

In certain embodiments, A³ is a 5-6 membered heteroaryl optionally substituted with 1, 2, or 3 substituents independently selected from the group consisting of halogen, C₁₋₆ alkyl, C₁₋₆ hydroxyalkyl, and C₁₋₄ alkoxyl. In certain embodiments, A³ is pyridinyl, pyridazinyl, pyrimidinyl, pyrazinyl, pyrazolyl, imidazolyl, or oxazolyl, each of which is optionally substituted with 1, 2, or 3 substituents independently selected from the group consisting of halogen, C₁₋₆ alkyl, C₁₋₆ hydroxyalkyl, and C₁₋₄ alkoxyl.

In certain embodiments, A³ is pyridinyl substituted with 1, 2, or 3 substituents independently selected from the group consisting of halogen, C₁₋₆ alkyl, C₁₋₆ hydroxyalkyl, and C₁₋₄ alkoxyl. In certain embodiments, A³ is pyridinyl substituted with (i) halogen and (ii) C₁₋₆ alkyl or C₁₋₄ alkoxyl. In certain embodiments, A³ is pyridinyl substituted with (i) halogen and (ii) C₁₋₄ alkoxyl. In certain embodiments, A³ is one of the following:

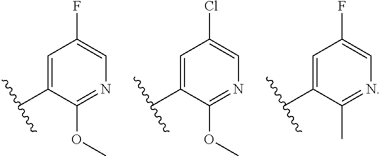

The description above describes multiple embodiments relating to compounds of Formula I-A. The patent application specifically contemplates all combinations of the embodiments.

Another aspect of the invention provides a compound represented by Formula I-B:

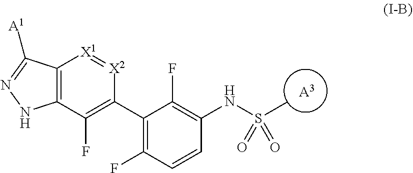

or a pharmaceutically acceptable salt thereof; wherein:
X¹ and X² are independently C(R²) or N, wherein X¹ is N and X² is C(R²), X¹ is C(R²) and X² is N, or both X¹ and X² are C(R²)
R² represents independently for each occurrence hydrogen or C₁₋₄ alkyl;
R³ and R⁴ each represent independently for each occurrence hydrogen or C₁₋₄ alkyl;
R⁵ is hydrogen or C₁₋₄ alkyl;
R⁶ is C₁₋₆ alkyl, C₁₋₆ hydroxyalkyl, C₃₋₆ cycloalkyl, 4-7 membered heterocyclyl, —(C₁₋₆ alkylene)-(C₃₋₆ cycloalkyl), —(C₁₋₆ alkylene)-(5-10 membered heterocycloalkyl), —(C₁₋₆ alkylene)-S(O)₂—(C₁₋₆ alkyl), —(C₁₋₆ alkylene)-N(R³)(R⁴), —(C₁₋₆ alkylene)-CN, wherein the C₁₋₆ alkyl, C₃₋₆ cycloalkyl, 4-7 membered heterocyclyl, —(C₁₋₆ alkylene)-(C₃₋₆ cycloalkyl), and —(C₁₋₆ alkylene)-(5-10 membered heterocycloalkyl) may be optionally substituted with 1, 2, or 3 substituents independently selected from the group consisting of cyano, halogen, hydroxyl, oxo, and —N(R³)(R⁴), and wherein if the 4-7 membered heterocyclyl and —(C₁₋₆ alkylene)-(5-10 membered heterocycloalkyl) contain a suitable ring nitrogen atom, that ring nitrogen may be optionally substituted by C₁₋₃ alkyl or —C(O)—C₁₋₃ alkyl;
R⁷ is C₁₋₄ alkyl;
A¹ is —C(O)N(R⁵)(R⁶), —N(R⁵)C(O)(R⁷), or 5-6 membered heterocyclyl; and
A³ is —CH₂—(C₃₋₆ cycloalkyl), phenyl, 5-10 membered heterocyclyl, and 7-10 membered bicyclic carbocyclyl, each of which is optionally substituted with 1, 2, or 3 substituents independently selected from the group consisting of C₁₋₆ hydroxyalkyl, C₁₋₄ fluoroalkoxyl, cyano, halogen, hydroxyl, and C₁₋₄ alkoxyl, wherein if the 5-10 membered heterocyclyl contains a suitable ring nitrogen atom, that ring nitrogen may be optionally substituted by C₁₋₃ alkyl.

The definitions of variables in Formula I-B above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-A.

In certain embodiments, X¹ and X² are both C(R²). In certain embodiments, R² represents independently for each occurrence is hydrogen or CH₃.

In certain embodiments, X¹ is C(R²) and X² is N. In certain embodiments, R² is hydrogen.

In certain embodiments, A¹ is —C(O)N(R⁵)(R⁶). In certain embodiments, R⁶ is C₁₋₆ alkyl, C₁₋₆ hydroxyalkyl, C₃₋₆ cycloalkyl, 4-7 membered heterocyclyl, —(C₁₋₆ alkylene)-(C₃₋₆ cycloalkyl), —(C₁₋₆ alkylene)-(5-10 membered heterocycloalkyl), —(C₁₋₆ alkylene)-S(O)₂—(C₁₋₆ alkyl), —(C₁₋₆ alkylene)-NH₂, —(C₁₋₆ alkylene)-CN, wherein the C₁₋₆ alkyl, C₃₋₆ cycloalkyl, 4-7 membered heterocyclyl, —(C₁₋₆ alkylene)-(C₃₋₆ cycloalkyl), and —(C₁₋₆ alkylene)-(5-10 membered heterocycloalkyl) may be optionally substituted with 1, 2, or 3 substituents independently selected from the group consisting of cyano, fluoro, hydroxyl, oxo, and NH₂, and wherein if the 4-7 membered heterocyclyl and —(C₁₋₆ alkylene)-(5-10 membered heterocycloalkyl) contain a suitable ring nitrogen atom, that ring nitrogen may be optionally substituted by CH₃ or —C(O)CH₃.

In certain embodiments, R⁶ is CH₃, CH₂CH₃,

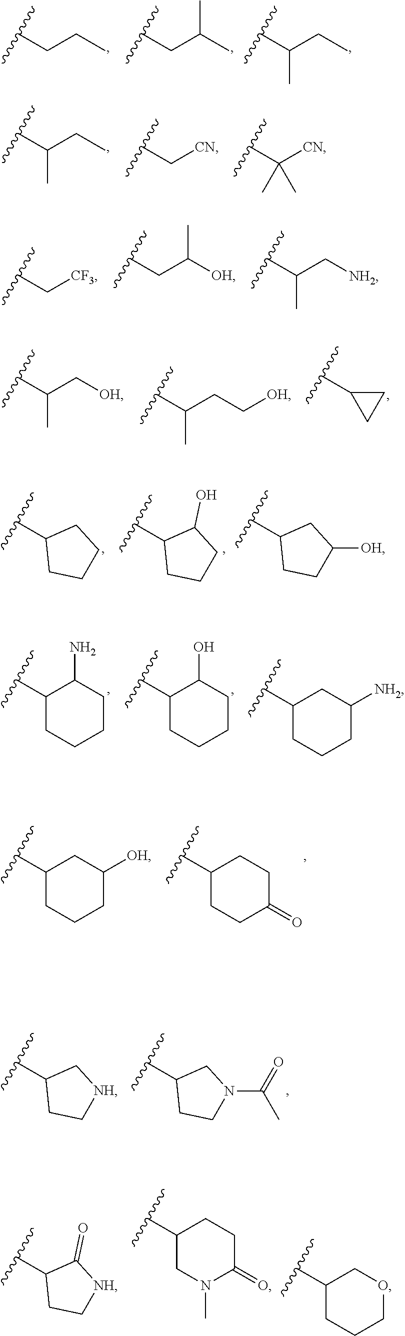

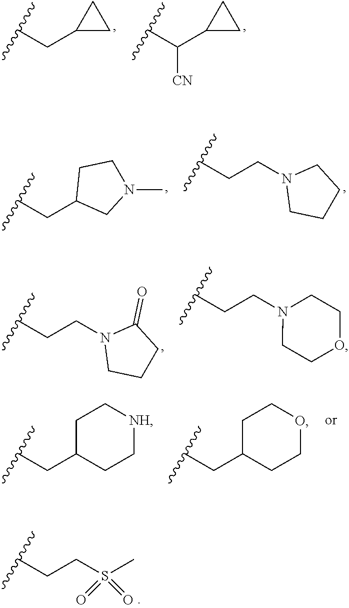

In certain embodiments, A¹ is —N(R⁵)C(O)(R⁷). In certain embodiments, R⁷ is CH₃.

In certain embodiments, R⁵ is hydrogen.

In certain embodiments, A¹ is 5-6 membered heterocyclyl. In certain embodiments, A¹ is

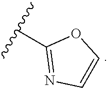 [SMILES: *c1ncco1]

In certain embodiments, A¹ is

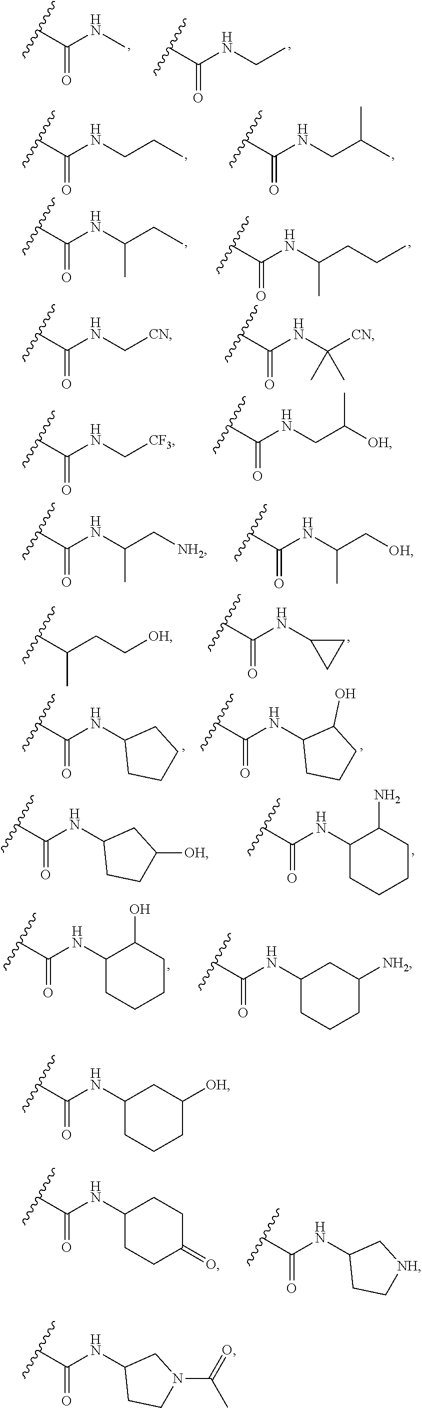

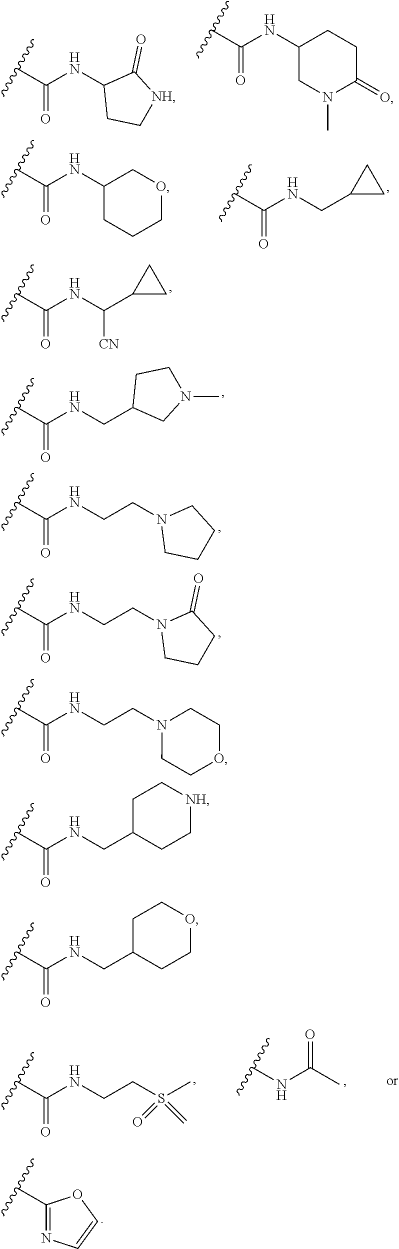

In certain embodiments, A³ is —CH₂—(C₃₋₆ cycloalkyl), phenyl, 5-10 membered heterocyclyl, and 7-10 membered bicyclic carbocyclyl, each of which is optionally substituted with 1, 2, or 3 substituents independently selected from the group consisting of chloro, fluoro, cyano, hydroxyl, CH₃, —CH₂OH, —O—CH₃, and —O—CHF₂, wherein if the 5-10 membered heterocyclyl contains a suitable ring nitrogen atom, that ring nitrogen may be optionally substituted by CH₃.

In certain embodiments, A³ is

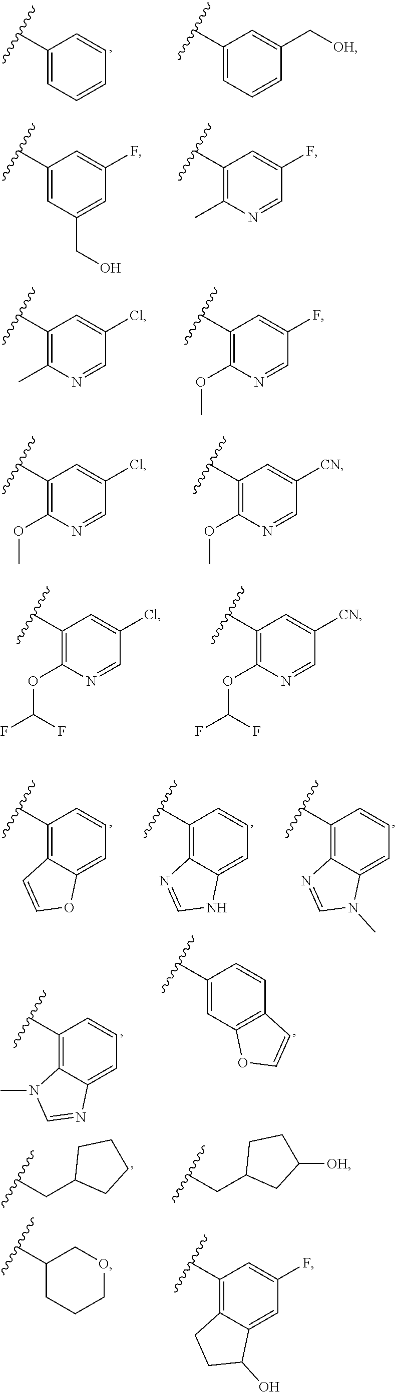

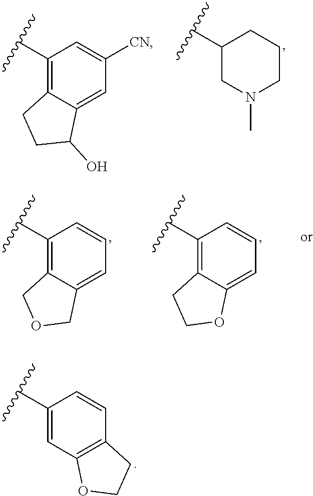

The description above describes multiple embodiments relating to compounds of Formula I-B. The patent application specifically contemplates all combinations of the embodiments.

Another aspect of the invention provides a compound represented by Formula II:

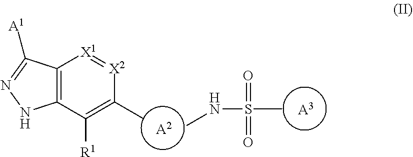

or a pharmaceutically acceptable salt thereof; wherein:

X¹ and X² are independently C(R²) or N, wherein X¹ is N and X² is C(R²), X¹ is C(R²) and X² is N, or both X¹ and X² are C(R²)

R¹ is halogen, hydrogen, C₁₋₄ alkyl, C₁₋₄ fluoroalkyl, or cyano;

R² represents independently for each occurrence hydrogen, halogen, C₁₋₄ alkyl, C₁₋₄ fluoroalkyl, cyano, C₁₋₄ alkoxyl, or hydroxyl;

R³ and R⁴ each represent independently hydrogen, C₁₋₄ alkyl, or C₃₋₇ cycloalkyl; or R³ and R⁴ are taken together with the nitrogen atom to which they are attached to form a 3-7 membered carbocyclyl or heterocyclyl;

R⁵ is hydrogen, C₁₋₄ alkyl, or C₃₋₇ cycloalkyl;

A¹ is —N(R³)(R⁴), —CO₂R⁵, C₁₋₄ alkyl, or hydrogen;

A² is phenylene or a 5-6 membered heteroarylene, each of which is optionally substituted with 1, 2, or 3 substituents independently selected from the group consisting of halogen, C₁₋₄ alkyl, C₁₋₄ haloalkyl, cyano, C₁₋₄ alkoxyl, C₃₋₅ cycloalkyl, and C₃₋₅ halocycloalkyl; and

A³ is one of the following:
5-10 membered unsaturated heterocyclyl optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, and —CO₂R⁵; or
phenyl substituted with 1, 2, or 3 substituents independently selected from the group consisting of halogen, C₁₋₆ alkyl, C₁₋₆ hydroxyalkyl, C₃₋₅ cycloalkyl, cyano, and C₁₋₄ alkoxyl.

The definitions of variables in Formula II above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula II.

In certain embodiments, X¹ and X² are independently C(R²). In certain embodiments, X¹ is C(R²), and X² is N. In certain embodiments, X¹ is N, and X² is C(R²).

In certain embodiments, R¹ is halogen. In certain embodiments, R¹ is fluoro.

In certain embodiments, R² is hydrogen. In certain embodiments, R³ and R⁴ are hydrogen. In certain embodiments, R², R³, and R⁴ are hydrogen.

In certain embodiments, R³ and R⁴ are taken together with the nitrogen atom to which they are attached to form a 3-7 membered ring.

In certain embodiments, A¹ is —CO₂R⁵. In certain embodiments, R⁵ is C₁₋₄ alkyl.

In certain embodiments, A¹ is —N(R³)(R⁴). In certain embodiments, A¹ is C₁₋₄ alkyl. In certain embodiments, A¹ is hydrogen.

In certain embodiments, A² is phenylene or a 5-6 membered heteroarylene, each of which is optionally substituted with 1, 2, or 3 substituents independently selected from the group consisting of halogen, C₁₋₄ alkyl, C₁₋₄ haloalkyl, and cyano. In certain embodiments, A² is phenylene, pyridinylene, pyridazinylene, pyrimidinylene, or pyrazinylene, each of which is optionally substituted with 1, 2, or 3 substituents independently selected from the group consisting of halogen, C₁₋₄ alkyl, C₁₋₄ haloalkyl, and cyano.

In certain embodiments, A² is phenylene optionally substituted with 1, 2, or 3 substituents independently selected from the group consisting of fluoro, chloro, C₁₋₄ alkyl, C₁-4 haloalkyl, and cyano. In certain embodiments, A² is pyridinylene, pyridazinylene, pyrimidinylene, or pyrazinylene, each of which is optionally substituted with 1, 2, or 3 substituents independently selected from the group consisting of halogen, C₁₋₄ alkyl, C₁₋₄ haloalkyl, and cyano. In certain embodiments, A² is pyridinylene optionally substituted with 1, 2, or 3 substituents independently selected from the group consisting of fluoro, chloro, C₁₋₄ alkyl, C₁₋₄ haloalkyl, and cyano.

In certain embodiments, A² is

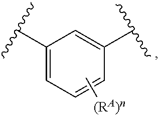

wherein R^{A} is fluoro; n is 1 or 2. In certain embodiments, A² is

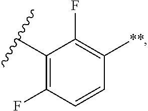

wherein ** is a bond to the sulfonamide nitrogen atom in Formula II.

In certain embodiments, A² is

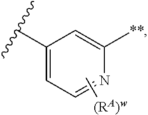

wherein R^{A} is fluoro; w is 1 or 2, and ** is a bond to the sulfonamide nitrogen atom in Formula II. In certain embodiments, A² is

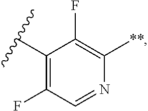

wherein ** is a bond to the sulfonamide nitrogen atom in Formula II.

In certain embodiments, A³ is a 5-10 membered unsaturated heterocyclyl optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, and oxo. In certain embodiments, A³ is a 5-6 membered unsaturated heterocyclyl optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, and oxo. In certain embodiments, A³ is a 5-6 membered heteroaryl substituted with C₁₋₄ alkoxyl and 1 or 2 substituents independently selected from the group consisting of halogen, C₁₋₆ alkyl, C₁₋₆ hydroxyalkyl, and C₁₋₄ alkoxyl.

In certain embodiments, A³ is pyridinyl, pyridazinyl, pyrimidinyl, pyrazinyl, pyrazolyl, imidazolyl, or oxazolyl, each of which is optionally substituted with 1, 2, or 3 substituents independently selected from the group consisting of halogen, C₁₋₆ alkyl, C₁₋₆ hydroxyalkyl, and C₁₋₄ alkoxyl. In certain embodiments, A³ is pyridinyl substituted with 1, 2, or 3 substituents independently selected from the group consisting of halogen, C₁₋₆ alkyl, C₁₋₆ hydroxyalkyl, and C₁₋₄ alkoxyl. In certain embodiments, A³ is pyridinyl substituted with (i) halogen and (ii) C₁₋₄ alkoxyl. In certain embodiments, A³ is one of the following:

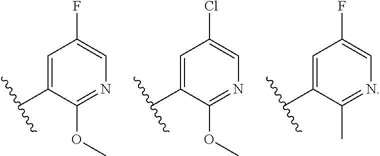

In certain embodiments, A³ is phenyl substituted with 1, 2, or 3 substituents independently selected from the group consisting of halogen, C₁₋₆ alkyl, C₁₋₆ hydroxyalkyl, C₃₋₅ cycloalkyl, cyano, and C₁₋₄ alkoxyl. In certain embodiments, A³ is phenyl substituted with 1, 2, or 3 substituents independently selected from the group consisting of halogen, C₁₋₆ hydroxyalkyl, C₃₋₅ cycloalkyl, cyano, and C₁₋₄ alkoxyl. In certain embodiments, A³ is phenyl substituted with (i) halogen and (ii) C₁₋₆ alkyl or C₁₋₄ alkoxyl. In certain embodiments, A³ is phenyl substituted with (i) halogen and (ii) C₁₋₄ alkoxyl.

The description above describes multiple embodiments relating to compounds of Formula II. The patent application specifically contemplates all combinations of the embodiments.

Another aspect of the invention provides a compound represented by Formula II-A:

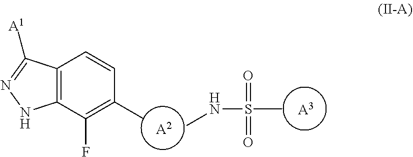

or a pharmaceutically acceptable salt thereof; wherein:

A¹ is —N(R³)(R⁴) or —CO₂R⁵;

R³ and R⁴ each represent independently hydrogen or C₁₋₄ alkyl;

R⁵ is hydrogen or C₁₋₄ alkyl;

A² is phenylene or pyridinylene, each of which is substituted with 1 or 2 substituents independently selected from the group consisting of fluoro, chloro, C₁₋₂ alkyl, C₁₋₂ haloalkyl, and cyano; and

A³ is one of the following:
5-10 membered unsaturated heterocyclyl optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, and C₁₋₄ alkoxyl; or
phenyl substituted with 1, 2, or 3 substituents independently selected from the group consisting of halogen, C₁₋₆ alkyl, C₁₋₆ hydroxyalkyl, C₃₋₅ cycloalkyl, cyano, and C₁₋₄ alkoxyl.

The definitions of variables in Formula II-A above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula II-A.

In certain embodiments, A¹ is —CO₂R⁵. In certain embodiments, R⁵ is C₁₋₄ alkyl.

In certain embodiments, A¹ is —N(R³)(R⁴). In certain embodiments, R³ and R⁴ are hydrogen. In certain embodiments, R³ and R⁴ are hydrogen or methyl.

In certain embodiments, A² is phenylene substituted with 1 or 2 substituents independently selected from the group consisting of fluoro, chloro, C₁₋₂ alkyl, C₁₋₂ haloalkyl, and cyano. In certain embodiments, A² is pyridinylene substituted with 1 or 2 substituents independently selected from the group consisting of fluoro, chloro, C₁₋₂ alkyl, C₁₋₂ haloalkyl, and cyano.

In certain embodiments, A² is

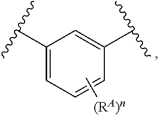

wherein R^{A} is fluoro; n is 1 or 2. In certain embodiments, A² is

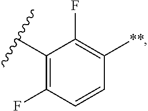

wherein ** is a bond to the sulfonamide nitrogen atom in Formula II-A.

In certain embodiments, A² is

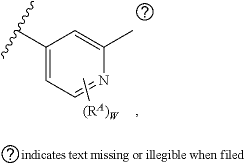

wherein R^{A} is fluoro; w is 1 or 2, and ** is a bond to the sulfonamide nitrogen atom in Formula II-A. In certain embodiments, A² is

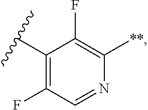

wherein ** is a bond to the sulfonamide nitrogen atom in Formula II-A.

In certain embodiments, A³ is a 5-10 membered unsaturated heterocyclyl optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, and C₁₋₄ alkoxyl. In certain embodiments, A³ is a 5-6 membered unsaturated heterocyclyl optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, and C₁₋₄ alkoxyl. In certain embodiments, A³ is a 5-6 membered heteroaryl substituted with C₁₋₄ alkoxyl and 1 or 2 substituents independently selected from the group consisting of halogen, C₁₋₆ alkyl, C₁₋₆ hydroxyalkyl, and C₁₋₄ alkoxyl.

In certain embodiments, A³ is pyridinyl, pyridazinyl, pyrimidinyl, pyrazinyl, pyrazolyl, imidazolyl, or oxazolyl, each of which is optionally substituted with 1, 2, or 3 substituents independently selected from the group consisting of halogen, C₁₋₆ alkyl, C₁₋₆ hydroxyalkyl, and C₁₋₄ alkoxyl. In certain embodiments, A³ is pyridinyl substituted with 1, 2, or 3 substituents independently selected from the group consisting of halogen, C₁₋₆ alkyl, C₁₋₆ hydroxyalkyl, and C₁₋₄ alkoxyl. In certain embodiments, A³ is pyridinyl substituted with (i) halogen and (ii) C₁₋₄ alkoxyl. In certain embodiments, A³ is one of the following:

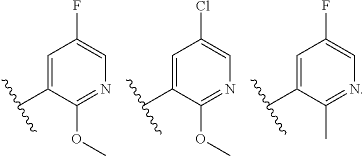

In certain embodiments, A³ is phenyl substituted with 1, 2, or 3 substituents independently selected from the group consisting of halogen, C₁₋₆ alkyl, C₁₋₆ hydroxyalkyl, C₃₋₅ cycloalkyl, cyano, and C₁₋₄ alkoxyl. In certain embodiments, A³ is phenyl substituted with 1, 2, or 3 substituents independently selected from the group consisting of halogen, C₁₋₆ hydroxyalkyl, C₃₋₅ cycloalkyl, cyano, and C₁₋₄ alkoxyl. In certain embodiments, A³ is phenyl substituted with (i) halogen and (ii) C₁₋₆ alkyl or C₁₋₄ alkoxyl. In certain embodiments, A³ is phenyl substituted with (i) halogen and (ii) C₁₋₄ alkoxyl.

The description above describes multiple embodiments relating to compounds of Formula II-A. The patent application specifically contemplates all combinations of the embodiments.

In certain other embodiments, the compound is one of the compounds listed in Table 1 below, or a pharmaceutically acceptable salt thereof. In certain embodiments, the compound is one of the compounds listed in Table 1 below. In certain other embodiments, the compound is one of the compounds listed in Table 2 below, or a pharmaceutically acceptable salt thereof. In certain embodiments, the compound is one of the compounds listed in Table 2 below. In certain other embodiments, the compound is one of the compounds listed in Tables 1-4 or 8 herein, or a pharmaceutically acceptable salt thereof. In certain other embodiments, the compound is one of the compounds listed in Tables 1-4 or 8 herein. In certain other embodiments, the compound is one of the compounds listed in Tables 3, 4, or 8 herein, or a pharmaceutically acceptable salt thereof. In certain other embodiments, the compound is one of the compounds listed in Tables 3, 4, or 8 herein. In certain other embodiments, the compound is one of the compounds listed in any one of Tables 5-7 and 9 herein, or a pharmaceutically acceptable salt thereof. In certain other embodiments, the compound is one of the compounds listed in any one of Tables 5-7 and 9 herein.

In certain embodiments, the compound is selected from the group consisting of:
6-(3-benzenesulfonamido-2,6-difluorophenyl)-7-fluoro-N-methyl-1H-indazole-3-carboxamide;
6-[2,6-Difluoro-3-[3-(hydroxymethyl)benzenesulfonamido]phenyl]-7-fluoro-N-methyl-1H-indazole-3-carboxamide;
6-[2,6-difluoro-3-[3-fluoro-5-(hydroxymethyl)benzenesulfonamido]phenyl]-7-fluoro-N-methyl-1H-indazole-3-carboxamide;
6-(3-amino-2-fluorophenyl)-7-fluoro-N-methyl-1-[[2-(trimethylsilyl)ethoxy]methyl]indazole-3-carboxamide;
N-(6-(3-(5-chloro-2-methoxypyridine-3-sulfonamido)-2,6-difluorophenyl)-7-fluoro-1H-indazol-3-yl)acetamide;
6-[3-[5-chloro-2-(difluoromethoxy)pyridine-3-sulfonamido]-2,6-difluorophenyl]-7-fluoro-N-methyl-1H-indazole-3-carboxamide;
6-[3-[5-Cyano-2-(difluoromethoxy)pyridine-3-sulfonamido]-2,6-difluorophenyl]-7-fluoro-N-methyl-1H-indazole-3-carboxamide;
6-[3-(5-chloro-2-methoxypyridine-3-sulfonamido)-2,6-difluorophenyl]-7-fluoro-N,4-dimethyl-1H-indazole-3-carboxamide;
6-[3-(5-Chloro-2-methoxypyridine-3-sulfonamido)-2,6-difluorophenyl]-7-fluoro-N-(2-methylpropyl)-1H-indazole-3-carboxamide;
6-(3-((5-Chloro-2-methoxypyridine)-3-sulfonamido)-2,6-difluorophenyl)-N-ethyl-7-fluoro-1H-indazole-3-carboxamide;
6-[3-(5-Chloro-2-methoxypyridine-3-sulfonamido)-2,6-difluorophenyl]-7-fluoro-N-(1-methyl-6-oxopiperidin-3-yl)-1H-indazole-3-carboxamide;
N-(1-acetylpyrrolidin-3-yl)-6-[3-(5-chloro-2-methoxypyridine-3-sulfonamido)-2,6-difluorophenyl]-7-fluoro-1H-indazole-3-carboxamide;
6-[3-(5-Chloro-2-methoxypyridine-3-sulfonamido)-2,6-difluorophenyl]-7-fluoro-N-(2-hydroxypropyl)-1H-indazole-3-carboxamide;
6-[3-(5-Chloro-2-methoxypyridine-3-sulfonamido)-2,6-difluorophenyl]-7-fluoro-N-(1-hydroxypropan-2-yl)-1H-indazole-3-carboxamide;
6-[3-(5-Chloro-2-methoxypyridine-3-sulfonamido)-2,6-difluorophenyl]-7-fluoro-N-(2-oxopyrrolidin-3-yl)-1H-indazole-3-carboxamide;
6-[3-(5-Chloro-2-methoxypyridine-3-sulfonamido)-2,6-difluorophenyl]-7-fluoro-N-(oxan-3-yl)-1H-indazole-3-carboxamide;
6-[3-(5-Chloro-2-methoxypyridine-3-sulfonamido)-2,6-difluorophenyl]-7-fluoro-N-(sec-butyl)-1H-indazole-3-carboxamide;
6-[3-(5-Chloro-2-methoxypyridine-3-sulfonamido)-2,6-difluorophenyl]-7-fluoro-N-(pyrrolidin-3-yl)-1H-indazole-3-carboxamide;
6-[3-(5-Chloro-2-methoxypyridine-3-sulfonamido)-2,6-difluorophenyl]-7-fluoro-N-propyl-1H-indazole-3-carboxamide;
6-[3-(5-Chloro-2-methoxypyridine-3-sulfonamido)-2,6-difluorophenyl]-7-fluoro-N-(pentan-2-yl)-1H-indazole-3-carboxamide;
6-[3-(5-chloro-2-methoxypyridine-3-sulfonamido)-2,6-difluorophenyl]-7-fluoro-N-(4-hydroxybutan-2-yl)-1H-indazole-3-carboxamide;
6-[3-(1-Benzofuran-4-sulfonamido)-2,6-difluorophenyl]-7-fluoro-N-methyl-1H-indazole-3-carboxamide;
6-[3-(1H-1,3-Benzodiazole-4-sulfonamido)-2,6-difluorophenyl]-7-fluoro-N-methyl-1H-indazole-3-carboxamide;
6-[2,6-Difluoro-3-(3-methyl-1,3-benzodiazole-4-sulfonamido)phenyl]-7-fluoro-N-methyl-1H-indazole-3-carboxamide;
5-chloro-N-[2,4-difluoro-3-[7-fluoro-3-(1,3-oxazol-2-yl)-1H-indazol-6-yl]phenyl]-2-methoxypyridine-3-sulfonamide;
6-[2,6-difluoro-3-(5-fluoro-2-methylpyridine-3-sulfonamido)phenyl]-7-fluoro-N-methyl-1H-indazole-3-carboxamide;
6-[2,6-Difluoro-3-(5-fluoro-2-methylpyridine-3-sulfonamido)phenyl]-7-fluoro-N-methyl-1H-pyrazolo[4,3-c]pyridine-3-carboxamide;
6-[3-(5-Chloro-2-methoxypyridine-3-sulfonamido)-2,6-difluorophenyl]-7-fluoro-N-methyl-1H-pyrazolo[4,3-c]pyridine-3-carboxamide;
N-[2,4-difluoro-3-[7-fluoro-3-(hydrazinecarbonyl)-1H-indazol-6-yl]phenyl]-1-benzofuran-6-sulfonamide;
6-[2,6-Difluoro-3-(1-methyl-1,3-benzodiazole-4-sulfonamido)phenyl]-7-fluoro-N-methyl-1H-indazole-3-carboxamide;
6-[2,6-Difluoro-3-(5-fluoro-2-methoxypyridine-3-sulfonamido)phenyl]-7-fluoro-N-methyl-1H-pyrazolo[4,3-c]pyridine-3-carboxamide;
6-[3-(5-Cyano-2-methoxypyridine-3-sulfonamido)-2,6-difluorophenyl]-7-fluoro-N-methyl-1H-pyrazolo[4,3-c]pyridine-3-carboxamide;
6-[3-(5-Chloro-2-methylpyridine-3-sulfonamido)-2,6-difluorophenyl]-7-fluoro-N-methyl-1H-pyrazolo[4,3-c]pyridine-3-carboxamide;
5-chloro-N-[2,4-difluoro-3-(7-fluoro-1H-indazol-6-yl)phenyl]-2-methoxypyridine-3-sulfonamide;
6-[3-(cyclopentylmethanesulfonamido)-2,6-difluorophenyl]-7-fluoro-N-methyl-1H-indazole-3-carboxamide;
6-[2,6-difluoro-3-(oxane-4-sulfonamido)phenyl]-7-fluoro-N-methyl-1H-indazole-3-carboxamide;
6-[2,6-Difluoro-3-(6-fluoro-1-hydroxy-2,3-dihydro-1H-indene-4-sulfonamido)phenyl]-7-fluoro-N-methyl-1H-indazole-3-carboxamide;
6-[2,6-difluoro-3-(1-methylpiperidine-3-sulfonamido)phenyl]-7-fluoro-N-methyl-1H-indazole-3-carboxamide;
6-[2,6-difluoro-3-[(3-hydroxycyclopentyl)methanesulfonamido]phenyl]-7-fluoro-N-methyl-1H-indazole-3-carboxamide;
6-[3-(5-Chloro-2-methoxypyridine-3-sulfonamido)-2,6-difluorophenyl]-N-cyclopentyl-7-fluoro-1H-indazole-3-carboxamide;
6-[3-(5-Chloro-2-methoxypyridine-3-sulfonamido)-2,6-difluorophenyl]-7-fluoro-N-(3-hydroxycyclohexyl)-1H-indazole-3-carboxamide;
6-[3-(5-Chloro-2-methoxypyridine-3-sulfonamido)-2,6-difluorophenyl]-N-cyclopropyl-7-fluoro-1H-indazole-3-carboxamide;
6-[3-(5-Chloro-2-ethoxypyridine-3-sulfonamido)-2,6-difluorophenyl]-7-fluoro-N-(morpholin-4-ylmethyl)-1H-indazole-3-carboxamide;
6-[3-(5-chloro-2-methoxypyridine-3-sulfonamido)-2,6-difluorophenyl]-7-fluoro-N-[2-(pyrrolidin-1-yl)ethyl]-1H-indazole-3-carboxamide;
6-[3-(5-Chloro-2-methoxypyridine-3-sulfonamido)-2,6-difluorophenyl]-7-fluoro-N-(2-methanesulfonylethyl)-1H-indazole-3-carboxamide;
6-[3-(5-Chloro-2-methoxypyridine-3-sulfonamido)-2,6-difluorophenyl]-N-(cyclopropylmethyl)-7-fluoro-1H-indazole-3-carboxamide;
6-[3-(5-Chloro-2-methoxypyridine-3-sulfonamido)-2,6-difluorophenyl]-7-fluoro-N-[2-(morpholin-4-yl)ethyl]-1H-indazole-3-carboxamide;
6-[3-(5-Chloro-2-methoxypyridine-3-sulfonamido)-2,6-difluorophenyl]-7-fluoro-N-(2-hydroxycyclopentyl)-1H-indazole-3-carboxamide;
6-[3-(5-Chloro-2-methoxypyridine-3-sulfonamido)-2,6-difluorophenyl]-7-fluoro-N-(2-hydroxycyclohexyl)-1H-indazole-3-carboxamide;
6-[3-(5-Chloro-2-methoxypyridine-3-sulfonamido)-2,6-difluorophenyl]-7-fluoro-N-(2-methylbut-3-yn-2-yl)-1H-indazole-3-carboxamide;
6-[3-(5-Chloro-2-methoxypyridine-3-sulfonamido)-2,6-difluorophenyl]-7-fluoro-N-(2,2,2-trifluoroethyl)-1H-indazole-3-carboxamide;
6-[3-(5-Chloro-2-methoxypyridine-3-sulfonamido)-2,6-difluorophenyl]-N-[cyano(cyclopropyl)methyl]-7-fluoro-1H-indazole-3-carboxamide;
6-[3-(5-Chloro-2-methoxypyridine-3-sulfonamido)-2,6-difluorophenyl]-7-fluoro-N-(prop-2-yn-1-yl)-1H-indazole-3-carboxamide;
6-[3-(5-Chloro-2-methoxypyridine-3-sulfonamido)-2,6-difluorophenyl]-7-fluoro-N-(piperidin-3-ylmethyl)-1H-indazole-3-carboxamide;
N-(2-Aminocyclohexyl)-6-[3-(5-chloro-2-methoxypyridine-3-sulfonamido)-2,6-difluorophenyl]-7-fluoro-1H-indazole-3-carboxamide;
N-(1-Aminopropan-2-yl)-6-[3-(5-chloro-2-methoxypyridine-3-sulfonamido)-2,6-difluorophenyl]-7-fluoro-1H-indazole-3-carboxamide;
6-[3-(5-Chloro-2-methoxypyridine-3-sulfonamido)-2,6-difluorophenyl]-7-fluoro-N-(4-oxocyclohexyl)-1H-indazole-3-carboxamide;
6-[3-(5-Chloro-2-methoxypyridine-3-sulfonamido)-2,6-difluorophenyl]-7-fluoro-N-(3-hydroxycyclopentyl)-1H-indazole-3-carboxamide;
N-(3-Aminocyclohexyl)-6-[3-(5-chloro-2-methoxypyridine-3-sulfonamido)-2,6-difluorophenyl]-7-fluoro-1H-indazole-3-carboxamide;
6-[3-(5-Chloro-2-methoxypyridine-3-sulfonamido)-2,6-difluorophenyl]-7-fluoro-N-[2-(2-oxopyrrolidin-1-yl)ethyl]-1H-indazole-3-carboxamide;
6-[3-(5-Chloro-2-methoxypyridine-3-sulfonamido)-2,6-difluorophenyl]-7-fluoro-N-[(1-methylpyrrolidin-3-yl)methyl]-1H-indazole-3-carboxamide;
6-[3-(1,3-dihydro-2-benzofuran-4-sulfonamido)-2,6-difluorophenyl]-7-fluoro-N-methyl-1H-indazole-3-carboxamide;
6-[3-(2,3-dihydro-1-benzofuran-6-sulfonamido)-2,6-difluorophenyl]-7-fluoro-N-methyl-1H-indazole-3-carboxamide;
6-[3-(2,3-Dihydro-1-benzofuran-6-sulfonamido)-2,6-difluorophenyl]-7-fluoro-N-methyl-1-[[2-(trimethylsilyl)ethoxy]methyl]indazole-3-carboxamide;
6-[3-(6-cyano-1-hydroxy-2,3-dihydro-1H-indene-4-sulfonamido)-2,6-difluorophenyl]-7-fluoro-N-methyl-1H-indazole-3-carboxamide;
6-[2,6-difluoro-3-(6-fluoro-1-hydroxy-2,3-dihydro-1H-indene-4-sulfonamido)phenyl]-7-fluoro-N-methyl-1H-pyrazolo[4,3-c]pyridine-3-carboxamide;
   or a pharmaceutically acceptable salt thereof.

TABLE 1

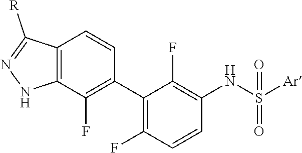

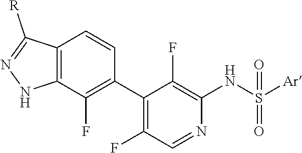

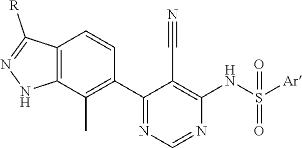

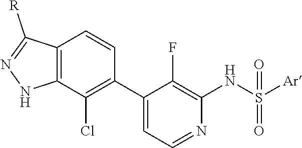

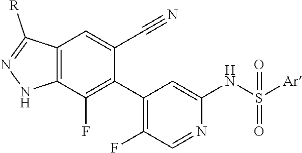

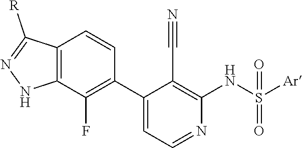

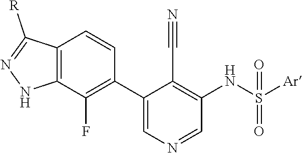

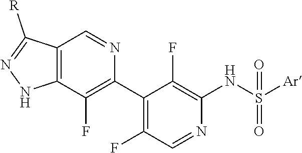

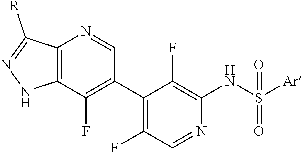

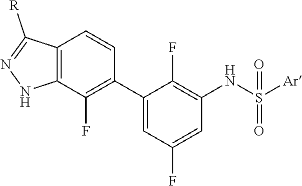

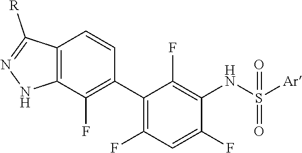

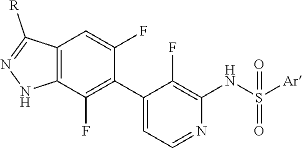

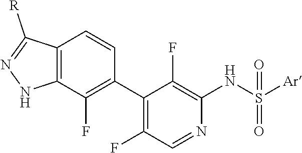

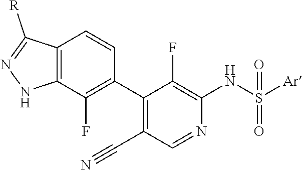

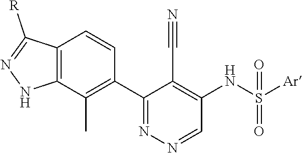

| No. | R | AR′ |
|---|---|---|
| I-1 | 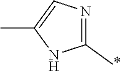 | 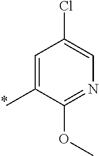 |
| I-2 | 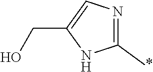 | 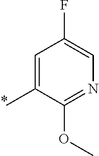 |
| I-3 | 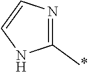 | 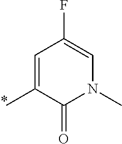 |
| I-4 | 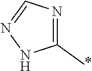 | 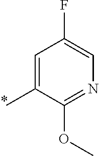 |
| I-5 | 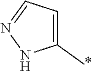 | 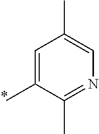 |
| I-6 | 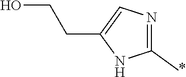 | 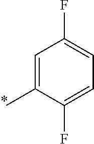 |
| I-7 | 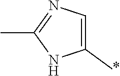 | 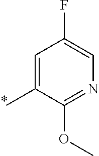 |
| I-8 | 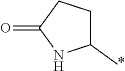 | 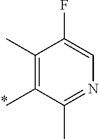 |
| I-9 | 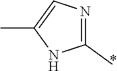 | 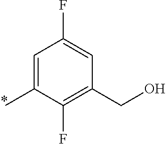 |
| I-10 | 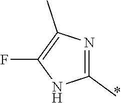 | 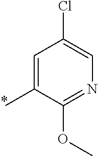 |
| I-11 | 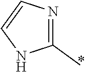 | 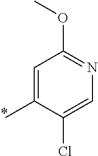 |
| I-12 | 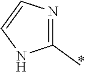 | 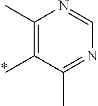 |
| I-13 | 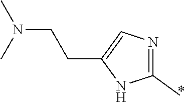 | 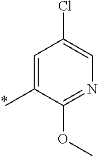 |
| I-14 | 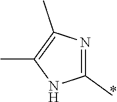 | 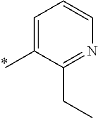 |
| I-15 | 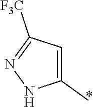 | 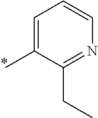 |
| I-16 | 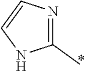 | 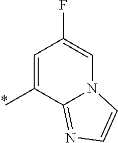 |
| I-17 | 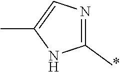 | 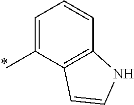 |
| I-18 | 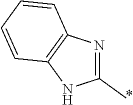 | 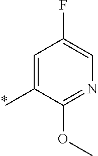 |
| I-19 | 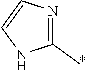 | 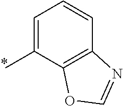 |
| I-20 | 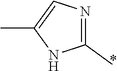 | 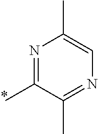 |
| I-21 | 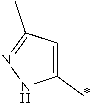 | 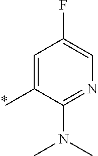 |
| I-22 | 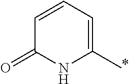 | 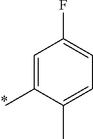 |

TABLE 2

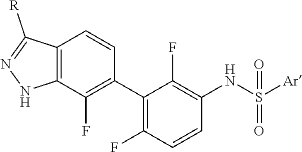

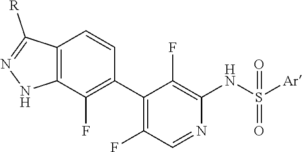

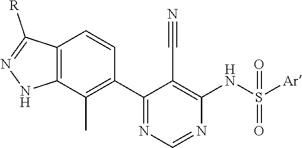

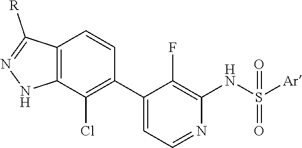

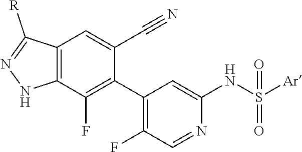

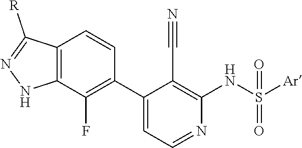

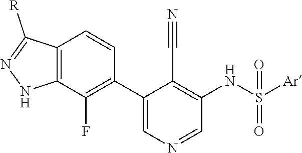

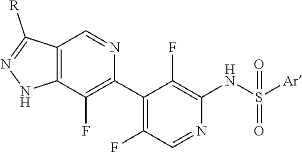

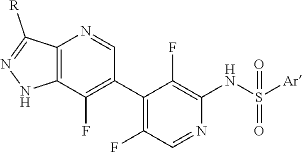

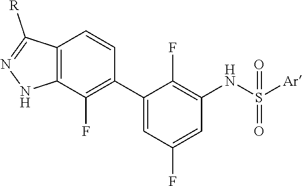

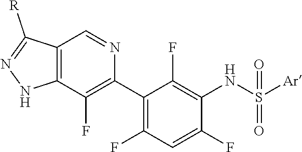

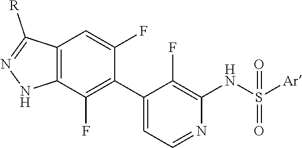

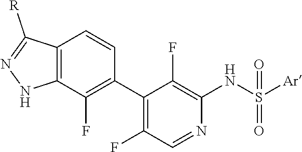

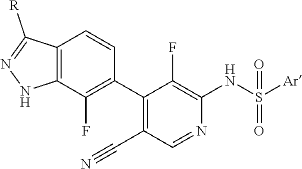

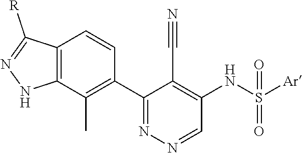

| No. | R | Ar′ |
|---|---|---|
| II-1 | 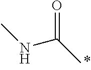 | 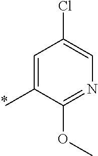 |
| II-2 | 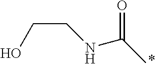 | 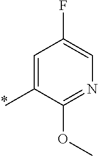 |
| II-3 | 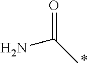 | 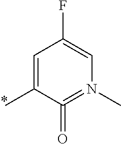 |
| II-4 | 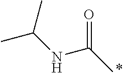 | 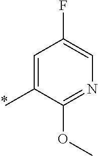 |
| II-5 | 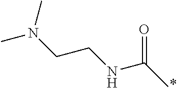 | 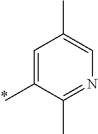 |
| II-6 | 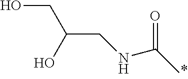 | 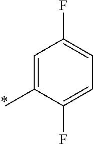 |
| II-7 | 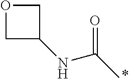 | 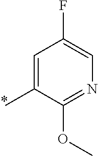 |
| II-8 | 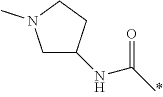 | 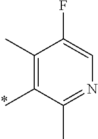 |
| II-9 | 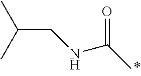 | 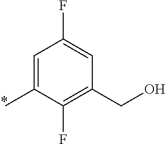 |
| II-10 | 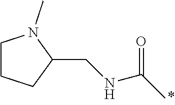 | 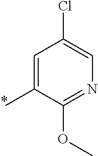 |
| II-11 | 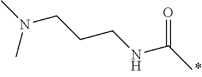 | 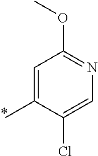 |
| II-12 | 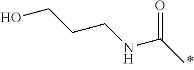 | 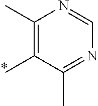 |
| II-13 | 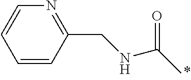 | 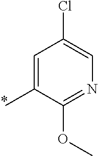 |
| II-14 | 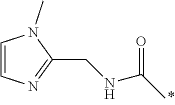 | 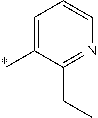 |
| II-15 | 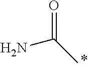 | 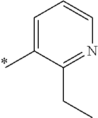 |
| II-16 | 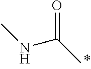 | 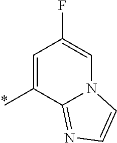 |
| II-17 | 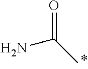 | 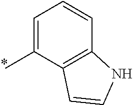 |
| II-18 | 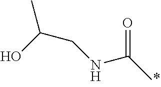 | 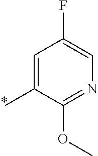 |
| II-19 | 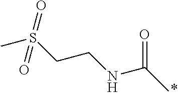 | 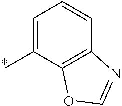 |
| II-20 | 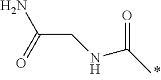 | 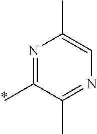 |
| II-21 | 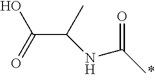 | 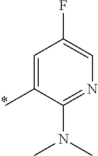 |
| II-22 | 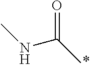 | 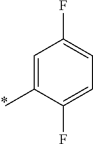 |

Methods for preparing compounds described herein are illustrated in the following synthetic Schemes. The Schemes are given for the purpose of illustrating the invention, and are not intended to limit the scope or spirit of the invention. Starting materials shown in the Schemes can be obtained from commercial sources or be prepared based on procedures described in the literature.

The synthetic route illustrated in Scheme 1 is a general method for preparing (aza)indazolyl-aryl sulfonamide and related compounds E, which includes compounds where one or more of A₁, A₂, A₃, or Cy are or contain one or more heteroatoms. The optionally substituted indazole or azaindazole A (prepared as described further below) can be treated, for example, with bis(pinacolato)diboron via Pd-mediated conditions to afford boronate B. Boronate B can then be coupled to a nitrogen-substituted aromatic or heteroaromatic halide Cₐ, C_{b}, or C_{c} to afford optionally substituted 6-aryl-indazole Dₐ, D_{b}, or D_{c}, and related compounds where one or more of A₁, A₂, A₃, or Cy are or contain one or more heteroatoms. The nitro-functionalized halide Cₐ and the biaryl Dₐ can be converted to aniline or aminoheterocycle C_{b} or D_{b}, for example, via hydrogenation with a transition metal-catalyst, or via a dissolving metal reduction in acid (such as Zn in acetic acid or a tin halide in a mineral acid). Coupling of aniline or aminoheterocycle (C_{b} or D_{b}) with a cyclic activated sulfonic acid derivative (CySO₂X, where X is either a halide or an appropriate leaving group such as a substituted phenol, alcohol or N-azole) under basic conditions (such as pyridine, a trialkyl amine, alkaline carbonate, alkaline hydroxide, DBU, LDA, or lithium hexamethyldisilazide) affords optionally substituted sulfonamide C_{c} or D_{c}. Removal of protecting group PG₁ (if used) with suitable conditions (for example, acidic conditions for removing a THP, SEM, or Boc moiety) affords (aza)indazolyl-aryl sulfonamide E, and related compounds where one or more of A₁, A₂, A₃, or Cy are or contain one or more heteroatoms. In cases where R₃ is an amide, R₃ may alternatively be taken through this sequence as an ester, and converted to the final amide E by treating the ester or its carboxylic acid with an amine with a suitable amide coupling reagent (DCC, PyBOP, DCI, etc.) or treating the ester with the amine with heat.

The reaction procedures in Scheme 1 are contemplated to be amenable to preparing a wide variety of (aza)indazolyl-aryl sulfonamide and related compounds E having different substituents at the A₁, A₂, A₃, Rₐ, R_{b}, R_{c}, Cy, and R₃-positions. For example, numerous substituted 3-haloanilines or haloaminoheterocycles (C_{b}) are known in the literature, and/or are commercially available or readily prepared from dihalo- or nitro-aromatic/heteroaromatic compounds. Also, numerous substituted cyclic sulfonyl halides (CySO₂X) are known in the literature and/or are commercially available or readily prepared from anilines or aryl halides. Furthermore, if a functional group on a molecule would not be amenable to a reaction condition described in Scheme 1, it is contemplated that the functional group can first be protected using standard protecting group chemistry and strategies, and then the protecting group can be removed after completing the desired synthetic transformation. See, for example, Greene, T. W.; Wuts, P. G. M. Protective Groups in Organic Synthesis, 2^{nd} ed.; Wiley: New York, 1991, for further description of protecting chemistry and strategies. Also see, for example, “Comprehensive Organic Synthesis” (B. M. Trost & I. Fleming, eds., 1991-1992).

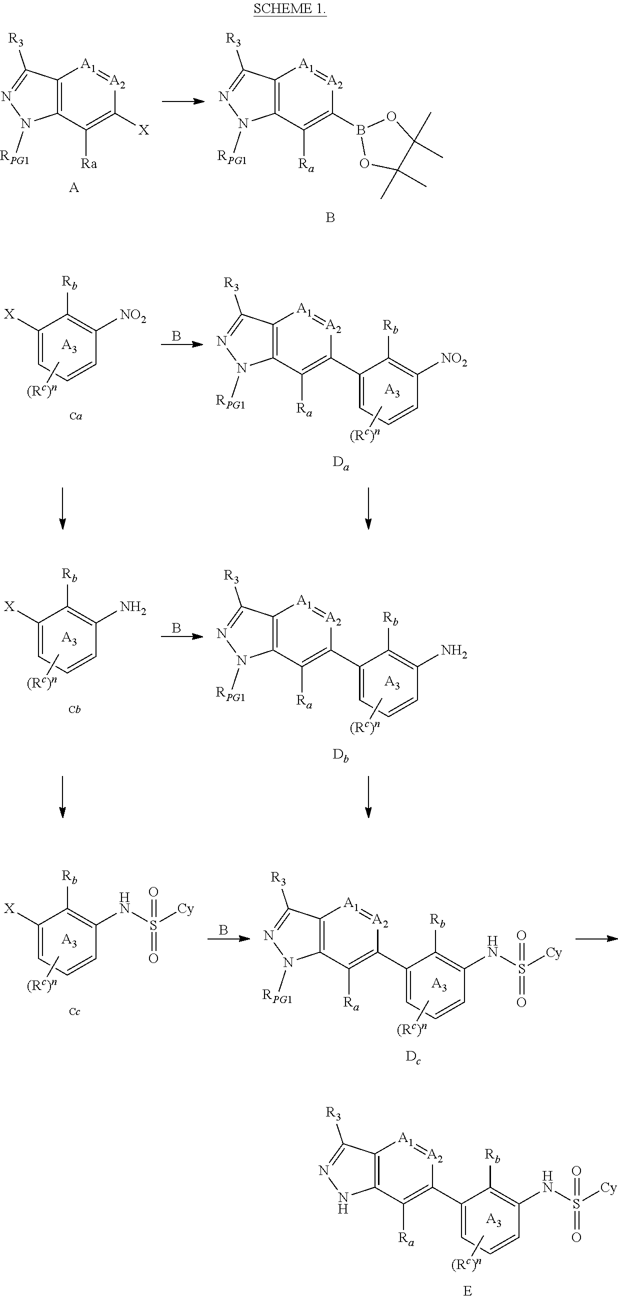

Scheme 2 illustrates general methods for preparing substituted indazoles or azaindazoles H. Substituted dihalobenzenes or substituted dihalopyridines or dihalopyrazines A, when not commercially available, can be prepared by lithiating between X₁ and X₂ by directed ortholithiation chemistry with, for example, LDA, nBuLi, or an MgNR₂ base followed by treatment with RaX. An ortho-directed metalation of A followed by treatment with CO₂ or DMF affords either directly the carboxylic acid B (R═OH) or an aldehyde B (R═H). Carboxylic acid B (R═OH) can be converted to Weinreb amide C using, for example, a suitable amide coupling reagent (e.g., DCC, PyBOP, DCI, etc.). Directed ortholithiations of N-protected azoles D (for example: pyrazoles, imidazoles, triazoles, or tetrazoles with THP, SEM, BOC or SO₂Ar as PG₁) affords lithium azole anion F. Alternatively, lithium azole anion F can be produced from a halogenated azole E by lithium-halogen exchange. Treatment of anion F with Weinreb amide C affords ketone G. Alternatively, treatment of anion F with aldehyde B (R═H) affords an intermediate carbinol, which can oxidized, for example, with manganese oxide or a chromium oxidant, to afford ketone G. Alternatively in select cases, dihalide A can be ortho-lithiated and treated with an ester I (X═OR) or a Weinreb amide I (X═NMeOMe) to afford ketone G. Treatment of ketone G with hydrazine (or a mono-protected hydrazine) affords, after optional protection, substituted indazole or azaindazole H.

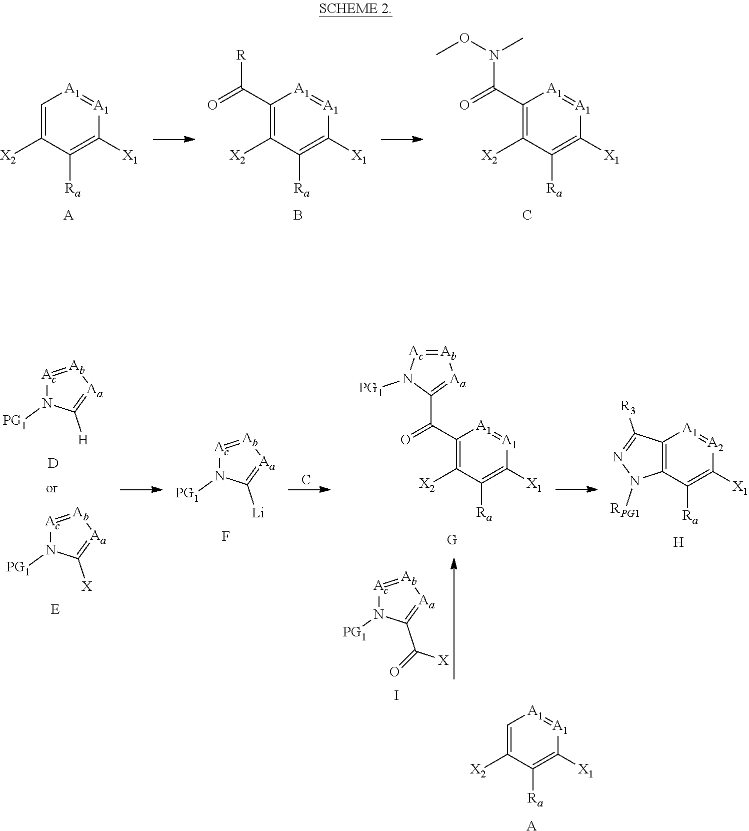

Scheme 3 illustrates a general method for preparing substituted indazoles or azaindazoles G and I. Hydrazone formation with the aldehyde A, followed by intramolecular cyclization with displacement of the halide X₂, affords indazole or azaindazole B. Halogenation of indazole or azaindazole B, for example, with iodine and base, affords iodide C, which can be protected to form iodide D. Pd-mediated addition of a cyanide affords nitrile E, which can be hydrolyzed in the presence of water or alcohol to afford the carboxlic acid or carboxylate ester F. The carboxylic acid F may also be formed directly from the iodide D via Pd-mediated insertion into CO₂. Standard amide formation with carboxylic acid F and a coupling reagent (e.g., DCC, PyBOP, DCI, etc.) or direct alcohol displacement of carboxylate ester F with an amine NH₂R₄ affords amide G. Nitrogen-protected azole boronate H (for example, pyrazoles, imidazoles, triazoles, or tetrazoles, with THP, SEM, BOC or SO₂Ar as PG₁), when not commercially available, may be produced, for example, by Pd-mediated coupling of bis(pinacolato)diboron with azole halide E depicted in Scheme 2, or from trimethyl borate and lithium anion F depicted in Scheme 2. Palladium-mediated cross-coupling of boronate H with iodide D affords indazole or azaindazole I.

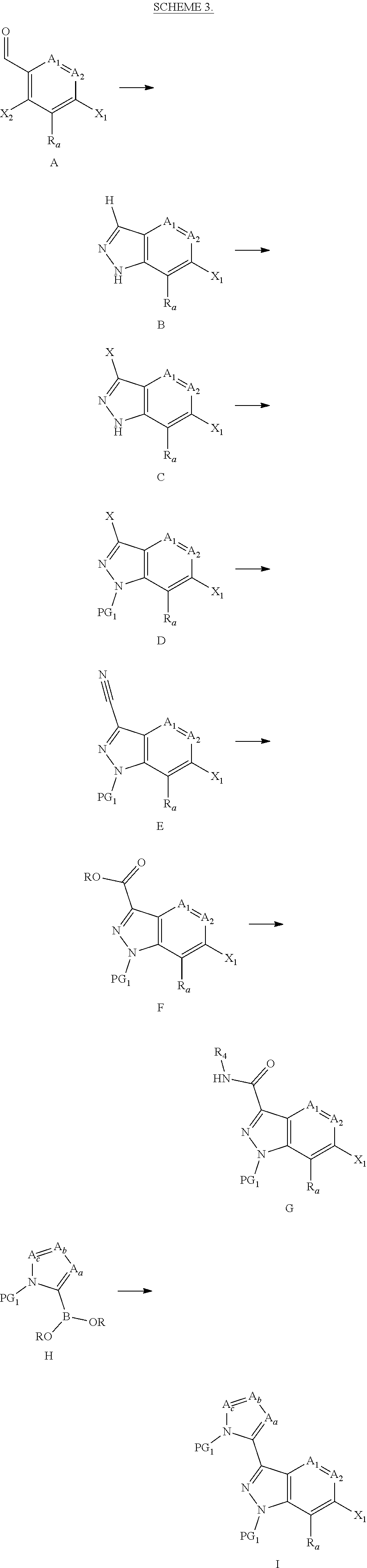

Scheme 4 illustrates another method for preparing substituted indazoles or azaindazoles. In select cases, a Fries reaction of resorcinol A with a 2-carboxylic acid, carbonyl halide, or acid anhydride of an azole in the presence of a Lewis acid affords ketone B. Treatment of the ketone with hydrazine affords hydroxylated azole C, which can be treated with triflic anhydride to afford triflate D (PG₁=Tf). Triflate D, may be used in place of the indazole or azaindazole halide A in the Pd-mediated couplings in Scheme 1.

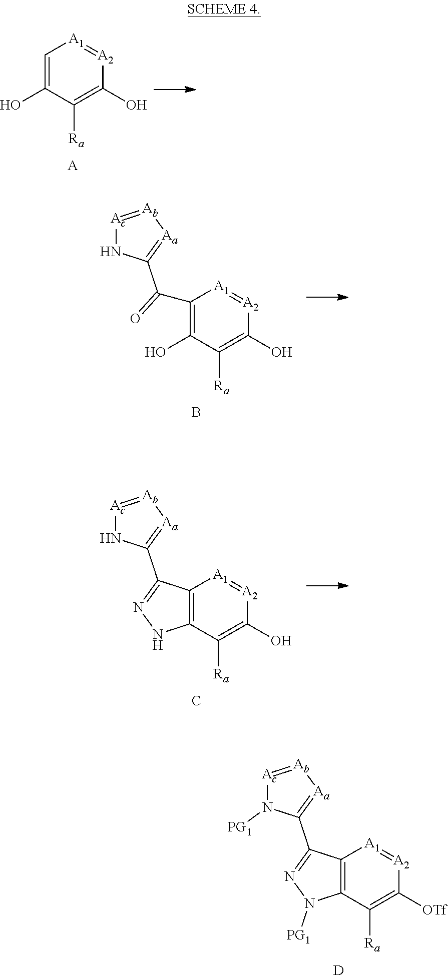

Scheme 5 illustrates a general procedure for preparing substituted aryl or heteroaryl sulfonyl halide C or activated sulfonate D, when not commercially available for use in Scheme 1. Pd-mediated addition of a benzyl thiol to aryl or heteroaryl halide A in the presence of base affords mercaptan B, which can be oxidized and converted directly to sulfonyl halide C by treatment with N-chlorosuccinimide in the presence of acetic acid or by treatment with sulfuryl chloride. Sulfonyl halide C may also be prepared by treating the aniline or aminoheterocycle E with nitrous acid or an alkyl nitrite to afford a diazonium salt which can then be converted to sulfonyl halide.

Select sulfonyl halides C do not possess good stability for storage and/or for conditions of coupling to anilines or amino-heterocycles. In those cases, it can be beneficial to convert the sulfonyl halide to an active sulfonate ester. For example, treatment of sulfonyl halide C with pentafluorophenol affords sulfonate D (Y═O, R_{act}═C₆H₅). In this instance, treatment of an aniline or amino-heterocycle (with LDA or LHMDS) forms a lithium anion which displaces the pentafluorophenol of sulfonate D to form the sulfonamide in THE at room temperature.

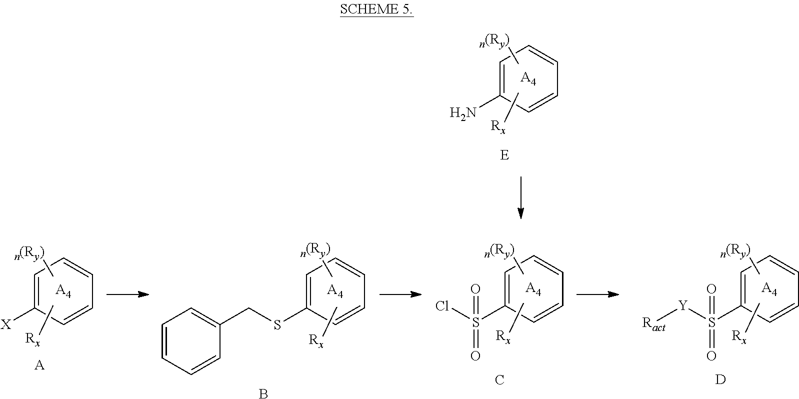

### II. Therapeutic Applications of (Aza)Indazolyl-Aryl Sulfonamide and Related Compounds

It is contemplated that (aza)indazolyl-aryl sulfonamide and related compounds described herein, such as a compound of Formula I, I-1, I-A, I-B, II, or II-A, or other compounds in Section I, provide therapeutic benefits to subjects suffering from cancer, neurodegenerative disease, and doxorubicin-induced cardiotoxicity. Accordingly, one aspect of the invention provides therapeutic methods for treating the foregoing diseases and conditions using (aza)indazolyl-aryl sulfonamide and related compounds described herein. Various aspects and embodiments of the therapeutic methods are described below Cancer

One aspect of the invention provides a method of treating cancer in a subject. The method comprises administering a therapeutically effective amount of a (aza)indazolyl-aryl sulfonamide or related compound described herein, such as a compound of Formula I, I-1, I-A, I-B, II, or II-A, or other compounds in Section I, to a subject in need thereof to treat the cancer. In certain embodiments, the particular compound of Formula I, I-1, I-A, I-B, II, or II-A, is a compound defined by one of the embodiments described above.

In certain embodiments, the cancer is a solid tumor, leukemia, or lymphoma. In certain embodiments, the cancer is colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, sweat gland carcinoma, sebaceous gland carcinoma, thyroid cancer, kidney cancer, uterus cancer, esophagus cancer, liver cancer, head cancer, neck cancer, throat cancer, mouth cancer, bone cancer, chest cancer, lymph node cancer, eye cancer, mesothelioma, an acoustic neuroma, oligodendroglioma, meningioma, neuroblastoma, retinoblastoma, leukemia, or lymphoma. In certain embodiments, the cancer is colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, leukemia, or lymphoma. In certain other embodiments, the cancer is colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, lung cancer, leukemia, bladder cancer, stomach cancer, cervical cancer, testicular cancer, skin cancer, rectal cancer, thyroid cancer, kidney cancer, uterus cancer, esophagus cancer, liver cancer, an acoustic neuroma, oligodendroglioma, meningioma, neuroblastoma, or retinoblastoma. In certain other embodiments, the cancer is small cell lung cancer, non-small cell lung cancer, melanoma, cancer of the central nervous system tissue, brain cancer, Hodgkin's lymphoma, non-Hodgkin's lymphoma, cutaneous T-Cell lymphoma, cutaneous B-Cell lymphoma, or diffuse large B-Cell lymphoma. In certain other embodiments, the cancer is breast cancer, colon cancer, small-cell lung cancer, non-small cell lung cancer, prostate cancer, renal cancer, ovarian cancer, leukemia, melanoma, or cancer of the central nervous system tissue. In certain other embodiments, the cancer is colon cancer, small-cell lung cancer, non-small cell lung cancer, renal cancer, ovarian cancer, renal cancer, or melanoma.

Additional exemplary cancers include fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, epithelial carcinoma, glioma, astrocytoma, medulloblastoma, and hemangioblastoma.

In certain embodiments, the cancer is a neuroblastoma, meningioma, hemangiopericytoma, multiple brain metastase, glioblastoma multiforms, glioblastoma, brain stem glioma, poor prognosis malignant brain tumor, malignant glioma, anaplastic astrocytoma, anaplastic oligodendroglioma, neuroendocrine tumor, rectal adeno carcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologic sarcoma, soft tissue sarcoma, scelroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waidenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, or leiomyoma.

### Neurodegenerative Disease

Another aspect of the invention provides a method of treating a neurodegenerative disease in a subject. The method comprises administering a therapeutically effective amount of a compound described herein, such as a compound of Formula I, I-1, I-A, I-B, II, or II-A, to a subject in need thereof to treat the neurodegenerative disease. In certain embodiments, the neurodegenerative disease is Alzheimer's disease, Parkinson's Disease, Huntington's Disease, amyotrophic lateral sclerosis, or spinocerebellar ataxia.

Aberrant autophagic processes contribute to neurodegenerative diseases. For example γ-secretase activity is enhanced in autophagic vacuoles through signal transduction mediated by GCN2 phosphorylation of the a subunit of eukaryotic initiation factor 2 (eIF2α) (see, e.g., Ohta, K. et al. in Autophagy 2010, 6, 345-352). The γ-secretase enhances amyloid-β synthesis and the progression of Alzheimer's disease. Accordingly, compounds having inhibitory activity towards GCN2 provide benefits to patients suffering from neurodegenerative diseases.

### Doxorubicin-Induced Cardiotoxicity

Another aspect of the invention provides a method of treating doxorubicin-induced cardiotoxicity in a subject. The method comprises administering a therapeutically effective amount of a compound described herein, such as a compound of Formula I, I-1, I-A, I-B, II, or II-A, to a subject in need thereof suffering from doxorubicin-induced cardiotoxicity, to thereby treat the doxorubicin-induced cardiotoxicity.

Another aspect of the invention provides a method of preventing doxorubicin-induced cardiotoxicity in a subject. The method comprises administering a therapeutically effective amount of a compound described herein, such as a compound of Formula I, I-1, I-A, I-B, II, or II-A, to a subject in need thereof that has received, or will receive, doxorubicin, to thereby prevent doxorubicin-induced cardiotoxicity.

Deficiency in GCN2 has been reported to ameliorate doxorubicin-induced cardiotoxicity. See, for example, Wang et al. in Redox Biology (2018) vol. 17, pages 25-34. Accordingly, compounds having inhibitory activity towards GCN2 provide benefits to patients suffering from or likely to suffer from doxorubicin-induced cardiotoxicity.

### Additional Aspects and Embodiments of Therapeutics Methods

In certain embodiments, the subject is a human.

Another aspect of the invention provides for the use of a compound described herein (such as a compound of Formula I, I-1, I-A, I-B, II, or II-A, or other compounds in Section I) in the manufacture of a medicament. In certain embodiments, the medicament is for treating a disorder described herein, such as cancer.

Another aspect of the invention provides for the use of a compound described herein (such as a compound of Formula I, I-1, I-A, I-B, II, or II-A, or other compounds in Section I) for treating a medical disorder, such a medical disorder described herein (e.g., cancer).

Further, it is contemplated that (aza)indazolyl-aryl sulfonamide and related compounds described herein, such as a compound of Formula I, I-1, I-A, I-B, II, or II-A, or other compounds in Section I, can inhibit the activity of GCN2. Accordingly, another aspect of the invention provides a method of inhibiting the activity of GCN2. The method comprises exposing a GCN2 to an effective amount of an (aza)indazolyl-aryl sulfonamide or related compound described herein, such as a compound of Formula I, I-1, I-A, I-B, II, or II-A, or other compounds in Section I, to inhibit GCN2 activity. In certain embodiments, the particular compound of Formula I, I-1, I-A, I-B, II, or II-A, is the compound defined by one of the embodiments described above.

### III. Combination Therapy

Another aspect of the invention provides for combination therapy. (aza)indazolyl-aryl sulfonamide and related compounds (e.g., a compound of Formula I, I-1, I-A, I-B, II, or II-A, or other compounds in Section I) or their pharmaceutically acceptable salts may be used in combination with additional therapeutic agents to treat medical disorders, such as a cancer.

Exemplary therapeutic agents that may be used as part of a combination therapy in treating cancer, include, for example, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and leutinizing hormone releasing factor.

Radiation therapy may also be used as part of a combination therapy.

An additional class of agents that may be used as part of a combination therapy in treating cancer is immune checkpoint inhibitors (also referred to as immune checkpoint blockers). Immune checkpoint inhibitors are a class of therapeutic agents that have the effect of blocking immune checkpoints. See, for example, Pardoll in Nature Reviews Cancer (2012) vol. 12, pages 252-264. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAB3, (v) B7-H3, (vi) B7-H4, and (vii) TIM3. The CTLA4 inhibitor Ipilumumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytoxic agents (e.g., tyrosine-kinase inhibitors).

Yet other agents that may be used as part of a combination therapy in treating cancer are agents which deplete amino acids or other nutrients, radiation, and agents that provoke the integrated stress response or that promote autophagy. Such agents may include aspariginase, argininase inhibitors of kinases such a b-Raf, and cytotoxic agents such as cis-platin.

Accordingly, another aspect of the invention provides a method of treating cancer in a patient, where the method comprises administering to the patient in need thereof (i) a therapeutically effective amount of a GCN2 inhibitor compound described herein and (ii) a second anti-cancer agent, in order to treat the cancer, where the second therapeutic agent may be one of the additional therapeutic agents described above (e.g., mitomycin, tretinoin, ribomustin, gemcitabine, an immune checkpoint inhibitor, or a monoclonal antibody agent that targets non-checkpoint targets) or one of the following:
an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor;
an agonist of OX40, CD137, CD40, GITR, CD27, HVEM, TNFRSF25, or ICOS;
a therapeutic antibody targeting one of the following: CD20, CD30, CD33, CD52, EpCAM, CEA, gpA33, a mucin, TAG-72, CAIX, PSMA, a folate-binding protein, a ganglioside, Le, VEGF, VEGFR, VEGFR2, integrin αVβ3, integrin α5β1, EGFR, ERBB2, ERBB3, MET, IGF1R, EPHA3, TRAILR1, TRAILR2, RANKL, FAP, tenascin, CD19, KIR, NKG2A, CD47, CEACAM1, c-MET, VISTA, CD73, CD38, BAFF, interleukin-1 beta, B4GALNT1, interleukin-6, and interleukin-6 receptor;
a cytokine selected from IL-12, IL-15, GM-CSF, and G-CSF;
a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine; or
a placental growth factor, an antibody-drug conjugate, an oncolytic virus, or an anti-cancer vaccine.

In certain embodiments, the second anti-cancer agent is an ALK Inhibitor. In certain embodiments, the second anti-cancer agent is an ALK Inhibitor comprising ceritinib or crizotinib. In certain embodiments, the second anti-cancer agent is an ATR Inhibitor. In certain embodiments, the second anti-cancer agent is an ATR Inhibitor comprising AZD6738 or VX-970. In certain embodiments, the second anti-cancer agent is an A2A Antagonist. In certain embodiments, the second anti-cancer agent is a Base Excision Repair Inhibitor comprising methoxyamine. In certain embodiments, the second anti-cancer agent is a Base Excision Repair Inhibitor, such as methoxyamine. In certain embodiments, the second anti-cancer agent is a Bcr-Abl Tyrosine Kinase Inhibitor. In certain embodiments, the second anti-cancer agent is a Bcr-Abl Tyrosine Kinase Inhibitor comprising dasatinib or nilotinib. In certain embodiments, the second anti-cancer agent is a Bruton's Tyrosine Kinase Inhibitor. In certain embodiments, the second anti-cancer agent is a Bruton's Tyrosine Kinase Inhibitor comprising ibrutinib. In certain embodiments, the second anti-cancer agent is a CDC7 Inhibitor. In certain embodiments, the second anti-cancer agent is a CDC7 Inhibitor comprising RXDX-103 or AS-141.

In certain embodiments, the second anti-cancer agent is a CHK1 Inhibitor. In certain embodiments, the second anti-cancer agent is a CHK1 Inhibitor comprising MK-8776, ARRY-575, or SAR-020106. In certain embodiments, the second anti-cancer agent is a Cyclin-Dependent Kinase Inhibitor. In certain embodiments, the second anti-cancer agent is a Cyclin-Dependent Kinase Inhibitor comprising palbociclib. In certain embodiments, the second anti-cancer agent is a DNA-PK Inhibitor. In certain embodiments, the second anti-cancer agent is a DNA-PK Inhibitor comprising MSC2490484A. In certain embodiments, the second anti-cancer agent is Inhibitor of both DNA-PK and mTOR. In certain embodiments, the second anti-cancer agent comprises CC-115.

In certain embodiments, the second anti-cancer agent is a DNMT1 Inhibitor. In certain embodiments, the second anti-cancer agent is a DNMT1 Inhibitor comprising decitabine, RX-3117, guadecitabine, NUC-8000, or azacytidine. In certain embodiments, the second anti-cancer agent comprises a DNMT1 Inhibitor and 2-chloro-deoxyadenosine. In certain embodiments, the second anti-cancer agent comprises ASTX-727.

In certain embodiments, the second anti-cancer agent is a HDAC Inhibitor. In certain embodiments, the second anti-cancer agent is a HDAC Inhibitor comprising OBP-801, CHR-3996, etinostate, resminostate, pracinostat, CG-200745, panobinostat, romidepsin, mocetinostat, belinostat, AR-42, ricolinostat, KA-3000, or ACY-241.

In certain embodiments, the second anti-cancer agent is a Hedgehog Signaling Pathway Inhibitor. In certain embodiments, the second anti-cancer agent is a Hedgehog Signaling Pathway Inhibitor comprising sonidegib or vismodegib. In certain embodiments, the second anti-cancer agent is an IDO Inhibitor. In certain embodiments, the second anti-cancer agent is an IDO Inhibitor comprising INCB024360. In certain embodiments, the second anti-cancer agent is a JAK Inhibitor. In certain embodiments, the second anti-cancer agent is a JAK Inhibitor comprising ruxolitinib or tofacitinib. In certain embodiments, the second anti-cancer agent is a mTOR Inhibitor. In certain embodiments, the second anti-cancer agent is a mTOR Inhibitor comprising everolimus or temsirolimus. In certain embodiments, the second anti-cancer agent is a MEK Inhibitor. In certain embodiments, the second anti-cancer agent is a MEK Inhibitor comprising cobimetinib or trametinib. In certain embodiments, the second anti-cancer agent is a MELK Inhibitor. In certain embodiments, the second anti-cancer agent is a MELK Inhibitor comprising ARN-7016, APTO-500, or OTS-167. In certain embodiments, the second anti-cancer agent is a MTH1 Inhibitor. In certain embodiments, the second anti-cancer agent is a MTH1 Inhibitor comprising (S)-crizotinib, TH287, or TH588.

In certain embodiments, the second anti-cancer agent is a PARP Inhibitor. In certain embodiments, the second anti-cancer agent is a PARP Inhibitor comprising MP-124, olaparib, BGB-290, talazoparib, veliparib, niraparib, E7449, rucaparb, or ABT-767. In certain embodiments, the second anti-cancer agent is a Phosphoinositide 3-Kinase Inhibitor. In certain embodiments, the second anti-cancer agent is a Phosphoinositide 3-Kinase Inhibitor comprising idelalisib. In certain embodiments, the second anti-cancer agent is an inhibitor of both PARP1 and DHODH (i.e., an agent that inhibits both poly ADP ribose polymerase 1 and dihydroorotate dehydrogenase).

In certain embodiments, the second anti-cancer agent is a Proteasome Inhibitor. In certain embodiments, the second anti-cancer agent is a Proteasome Inhibitor comprising bortezomib or carfilzomib. In certain embodiments, the second anti-cancer agent is a Topoisomerase-II Inhibitor. In certain embodiments, the second anti-cancer agent is a Topoisomerase-II Inhibitor comprising vosaroxin.

In certain embodiments, the second anti-cancer agent is a Tyrosine Kinase Inhibitor. In certain embodiments, the second anti-cancer agent is a Tyrosine Kinase Inhibitor comprising bosutinib, cabozantinib, imatinib or ponatinib. In certain embodiments, the second anti-cancer agent is a VEGFR Inhibitor. In certain embodiments, the second anti-cancer agent is a VEGFR Inhibitor comprising regorafenib. In certain embodiments, the second anti-cancer agent is a WEE1 Inhibitor. In certain embodiments, the second anti-cancer agent is a WEE1 Inhibitor comprising AZD1775.

In certain embodiments, the second anti-cancer agent is an agonist of OX40, CD137, CD40, GITR, CD27, HVEM, TNFRSF25, or ICOS. In certain embodiments, the second anti-cancer agent is a therapeutic antibody selected from the group consisting of rituximab, ibritumomab tiuxetan, tositumomab, obinutuzumab, ofatumumab, brentuximab vedotin, gemtuzumab ozogamicin, alemtuzumab, IGN101, adecatumumab, labetuzumab, huA33, pemtumomab, oregovomab, minetumomab, cG250, J591, Mov18, farletuzumab, 3F8, ch14.18, KW-2871, hu3S193, lgN311, bevacizumab, IM-2C6, pazopanib, sorafenib, axitinib, CDP791, lenvatinib, ramucirumab, etaracizumab, volociximab, cetuximab, panitumumab, nimotuzumab, 806, afatinib, erlotinib, gefitinib, osimertinib, vandetanib, trastuzumab, pertuzumab, MM-121, AMG 102, METMAB, SCH 900105, AVE1642, IMC-A12, MK-0646, R1507, CP 751871, KB004, IIIA-4, mapatumumab, HGS-ETR2, CS-1008, denosumab, sibrotuzumab, F19, 81C6, MEDI551, lirilumab, MEDI9447, daratumumab, belimumab, canakinumab, dinutuximab, siltuximab, and tocilizumab.

In certain embodiments, the second anti-cancer agent is a placental growth factor. In certain embodiments, the second anti-cancer agent is a placental growth factor comprising ziv-aflibercept. In certain embodiments, the second anti-cancer agent is an antibody-drug conjugate. In certain embodiments, the second anti-cancer agent is an antibody-drug conjugate selected from the group consisting of brentoxumab vedotin and trastuzumab emtransine.

In certain embodiments, the second anti-cancer agent is an oncolytic virus. In certain embodiments, the second anti-cancer agent is the oncolytic virus talimogene laherparepvec. In certain embodiments, the second anti-cancer agent is an anti-cancer vaccine. In certain embodiments, the second anti-cancer agent is an anti-cancer vaccine selected from the group consisting of a GM-CSF tumor vaccine, a STING/GM-CSF tumor vaccine, and NY-ESO-1. In certain embodiments, the second anti-cancer agent is a cytokine selected from IL-12, IL-15, GM-CSF, and G-CSF.

In certain embodiments, the second anti-cancer agent is a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine.

The doses and dosage regimen of the active ingredients used in the combination therapy may be determined by an attending clinician. In certain embodiments, the (aza)indazolyl-aryl sulfonamide or related compound (e.g., a compound of any one of Formula I, I-1, I-A, I-B, II, or II-A, or other compounds in Section I) and the additional therapeutic agent(s) are administered in doses commonly employed when such agents are used as monotherapy for treating the disorder. In other embodiments, the (aza)indazolyl-aryl sulfonamide or related compound (e.g., a compound of any one of Formula I, I-1, I-A, I-B, II, or II-A, or other compounds in Section I) and the additional therapeutic agent(s) are administered in doses lower than the doses commonly employed when such agents are used as monotherapy for treating the disorder. In certain embodiments, the (aza)indazolyl-aryl sulfonamide or related compound (e.g., a compound of any one of Formula I, I-1, I-A, I-B, II, or II-A, or other compounds in Section I) and the additional therapeutic agent(s) are present in the same composition, which is suitable for oral administration.

In certain embodiments, the (aza)indazolyl-aryl sulfonamide or related compound (e.g., a compound of any one of Formula I, I-1, I-A, I-B, II, or II-A, or other compounds in Section I) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

Another aspect of this invention is a kit comprising a therapeutically effective amount of the (aza)indazolyl-aryl sulfonamide or related compound (e.g., a compound of any one of Formula I, I-1, I-A, I-B, II, or II-A, or other compounds in Section I), a pharmaceutically acceptable carrier, vehicle or diluent, and optionally at least one additional therapeutic agent listed above.

### IV. Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising an (aza)indazolyl-aryl sulfonamide or related compound described herein in a therapeutically effective amount for the treatment of a medical disorder described herein.

### EXAMPLES

The invention now being generally described, will be more readily understood by reference to the following examples, which are included merely for purposes of illustration of certain aspects and embodiments of the present invention, and are not intended to limit the invention. Starting materials described herein can be obtained from commercial sources or may be readily prepared from commercially available materials using transformations known to those of skill in the art.

### Example 1—Synthesis of N-(2,4-difluoro-3-(7-fluoro-3-(1H-imidazol-2-yl)-1H-indazol-6-yl)phenyl)-4-methylbenzenesulfonamide

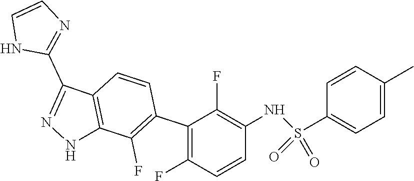 [SMILES: Cc1ccc(S(=O)(=O)Nc2ccc(F)c(-c3ccc4c(-c5ncc[nH]5)n[nH]c4c3F)c2F)cc1]

### Part I—Synthesis of Ethyl 1-((2-(Trimethylsilyl)ethoxy)methyl)-1H-imidazole-2-carboxylate

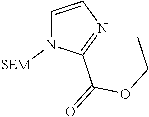 [SMILES: CCOC(=O)c1nccn1COCC[Si](C)(C)C]

In several small portions, 60% sodium hydride in mineral oil (1.03 g, 43 mmol) was added to a stirred solution of ethyl 1H-imidazole-2-carboxylate (5.0 g, 36 mmol) in THE (80 mL) at 0° C. After stirring an additional fifteen minutes at 0° C., 2-(trimethylsilyl)ethoxymethyl chloride (7.14 g, 43 mmol) was added dropwise at 0° C. The mixture was stirred an additional two hours at room temperature. Water was carefully added, and the mixture was extracted three times with ethyl acetate. The combined organic layers were washed with brine, dried (Na₂SO₄) and concentrated. The resulting residue was purified by MPLC eluting with a gradient of 0-33% ethyl acetate in petroleum ether to afford ethyl 1-((2-(trimethylsilyl)ethoxy)methyl)-1H-imidazole-2-carboxylate (8.0 g, 83%) as a light yellow oil.

### Part II—Synthesis of (4-Bromo-2,3-difluorophenyl)(1-((2-(trimethylsilyl)ethoxy)methyl)-1H-imidazol-2-yl)methanone

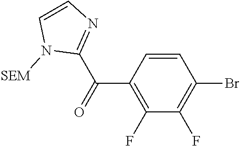 [SMILES: C[Si](C)(C)CCOCn1ccnc1C(=O)c1ccc(Br)c(F)c1F]

A 2.0 M solution of lithium diisopropyl amide (11.1 mL, 22.2 mmol) was added dropwise into a stirred solution of 1-bromo-2,3-difluorobenzene (4.28 g, 22.2 mmol) in THE (100 mL) at −78° C. Upon completion of the addition, the mixture was stirred for an additional hour at −78° C. A solution of ethyl 1-((2-(trimethylsilyl)ethoxy)methyl)-1H-imidazole-2-carboxylate (5.0 g, 18.5 mmol) in THE (5 mL) was added dropwise at −78° C. Upon completion of the addition, the mixture was stirred for an additional two hours at room temperature. The reaction was quenched by the addition of 1M HCl. The mixture was extracted three times with ethyl acetate. The combined organic layers were washed with brine, dried (Na₂SO₄) and concentrated. The resulting residue was purified by MPLC eluting with a gradient of 0-33% ethyl acetate in petroleum ether to afford (4-bromo-2,3-difluorophenyl)(1-((2-(trimethylsilyl)ethoxy)methyl)-1H-imidazol-2-yl)methanone (3.5 g, 45%) as a light yellow oil.

### Part III—Synthesis of 6-Bromo-7-fluoro-3-(1-((2-(trimethylsilyl)ethoxy)methyl)-1H-imidazol-2-yl)-1H-indazole

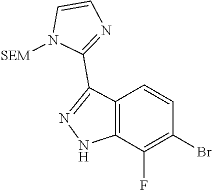 [SMILES: C[Si](C)(C)CCOCn1ccnc1-c1n[nH]c2c(F)c(Br)ccc12]

A mixture of (4-bromo-2,3-difluorophenyl)(1-((2-(trimethylsilyl)ethoxy)methyl)-1H-imidazol-2-yl)methanone (3.5 g, 8.4 mmol), xylene (30 mL), and 80% hydrazine hydrate (5.2 g) was stirred overnight at 140° C. and concentrated. The resulting residue was purified by MPLC eluting with a gradient of 0-33% ethyl acetate in petroleum ether to afford 6-bromo-7-fluoro-3-(1-((2-(trimethylsilyl)ethoxy)methyl)-1H-imidazol-2-yl)-1H-indazole (2.5 g, 72%) as a white solid.

### Part IV—Synthesis of 6-Bromo-7-fluoro-1-[[2-(trimethylsilyl)ethoxy]methyl]-3-(1-[[2-(trimethylsilyl)ethoxy]methyl]-1H-imidazol-2-yl)-1H-indazole

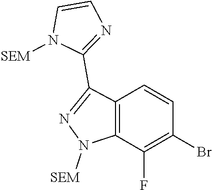 [SMILES: C[Si](C)(C)CCOCn1ccnc1-c1nn(COCC[Si](C)(C)C)c2c(F)c(Br)ccc12]

In several small portions, 60% sodium hydride in mineral oil (0.18 g, 7.50 mmol) was added to a stirred solution of 6-bromo-7-fluoro-3-(1-((2-(trimethylsilyl)ethoxy)methyl)-1H-imidazol-2-yl)-1H-indazole (2.5 g, 6.1 mmol) in THE (50 mL) at 0° C. After stirring an additional thirty minutes at 0° C., 2-(trimethylsilyl)ethoxymethyl chloride (1.22 g, 7.3 mmol) was added dropwise at 0° C. The resulting mixture was allowed to stir for an additional three hours at room temperature. Ice water was carefully added and the mixture was extracted three times with ethyl acetate. The combined organic layers were washed with brine, dried (Na₂SO₄) and concentrated. The resulting residue was purified by MPLC eluting with a gradient of 0-17% ethyl acetate in petroleum ether to afford 6-bromo-7-fluoro-1-[[2-(trimethylsilyl)ethoxy]methyl]-3-(1-[[2-(trimethylsilyl)ethoxy]methyl]-1H-imidazol-2-yl)-1H-indazole (2.5 g, 76%) as a light yellow oil.

### Part V—Synthesis of 7-Fluoro-6-(4,4,5,5-tetramethyl-1,3,2-dioxaborolan-2-yl)-1-((2-(trimethylsilyl)ethoxy)methyl)-3-(1-((2-(trimethylsilyl)ethoxy)methyl)-1H-imidazol-2-yl)-1H-indazole

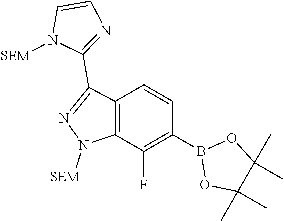 [SMILES: CC1(C)OB(c2ccc3c(-c4nccn4COCC[Si](C)(C)C)nn(COCC[Si](C)(C)C)c3c2F)OC1(C)C]

A mixture of 6-bromo-7-fluoro-1-[[2-(trimethylsilyl)ethoxy]methyl]-3-(1-[[2-(trimethylsilyl)ethoxy]methyl]-1H-imidazol-2-yl)-1H-indazole (2.5 g, 4.6 mmol), 4,4,5,5-tetramethyl-2-(4,4,5,5-tetramethyl-1,3,2-dioxaborolan-2-yl)-1,3,2-dioxaborolane (1.52 g, 6.0 mmol), dioxane (50 mL), potassium acetate (0.91 g, 9.2 mmol), and [1,1′-bis(diphenylphosphino)ferrocene]dichloropalladium(II) (377 mg, 0.46 mmol) was stirred overnight at 105° C. then concentrated. The resulting residue was purified by MPLC eluting with a gradient of 0-17% ethyl acetate in petroleum ether to afford 7-fluoro-6-(4,4,5,5-tetramethyl-1,3,2-dioxaborolan-2-yl)-1-((2-(trimethylsilyl)ethoxy)methyl)-3-(1-((2-(trimethylsilyl)ethoxy)methyl)-1H-imidazol-2-yl)-1H-indazole (2.2 g, 81%) as a light yellow oil.

### Part VI—Synthesis of 6-(2,6-Difluoro-3-nitrophenyl)-7-fluoro-1-((2-(trimethylsilyl)ethoxy)methyl)-3-(1-((2-(trimethylsilyl)ethoxy)methyl)-1H-imidazol-2-yl)-1H-indazole

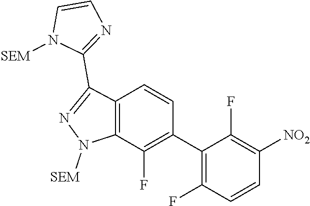 [SMILES: C[Si](C)(C)CCOCn1ccnc1-c1nn(COCC[Si](C)(C)C)c2c(F)c(-c3c(F)ccc([N+](=O)[O-])c3F)ccc12]

A mixture of 7-fluoro-6-(4,4,5,5-tetramethyl-1,3,2-dioxaborolan-2-yl)-1-((2-(trimethylsilyl)ethoxy)methyl)-3-(1-((2-(trimethylsilyl)ethoxy)methyl)-1H-imidazol-2-yl)-1H-indazole (2.5 g, 4.25 mmol), 2-bromo-1,3-difluoro-4-nitrobenzene (1.01 g, 4.25 mmol), toluene (30 mL), ethanol (10 mL), water (10 mL), potassium carbonate (1.76 g, 12.735 mmol), and tetrakis(triphenylphosphine)palladium(0) (0.49 g, 0.425 mmol) was stirred for three hours at 110° C. The mixture was concentrated. The resulting residue was purified by MPLC eluting with a gradient of 0-25% ethyl acetate in petroleum ether to afford 6-(2,6-difluoro-3-nitrophenyl)-7-fluoro-1-((2-(trimethylsilyl)ethoxy)methyl)-3-(1-((2-(trimethylsilyl)ethoxy)methyl)-1H-imidazol-2-yl)-1H-indazole (2.0 g, 76%) as a light yellow oil.

### Part VII—Synthesis of 2,4-Difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)aniline

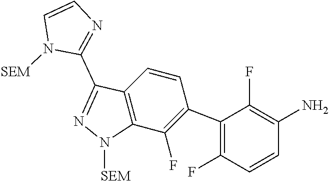 [SMILES: C[Si](C)(C)CCOCn1ccnc1-c1nn(COCC[Si](C)(C)C)c2c(F)c(-c3c(F)ccc(N)c3F)ccc12]

A mixture of 6-(2,6-difluoro-3-nitrophenyl)-7-fluoro-1-((2-(trimethylsilyl)ethoxy)methyl)-3-(1-((2-(trimethylsilyl)ethoxy)methyl)-1H-imidazol-2-yl)-1H-indazole (2.0 g, 3.2 mmol), ethyl acetate (40 mL), and 10% palladium on carbon (0.02 mmol) was stirred for two hours under an atmosphere of hydrogen. The mixture was filtered through Celite, and washed with ethyl acetate. The combined filtrate was concentrated. The resulting residue was purified by MPLC eluting with a gradient of 0-33% ethyl acetate in petroleum ether to afford 2,4-difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)aniline (1.5 g, 79%) as a light yellow solid.

### Part VIII—Synthesis of N-(2,4-Difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)phenyl)-4-methylbenzenesulfonamide

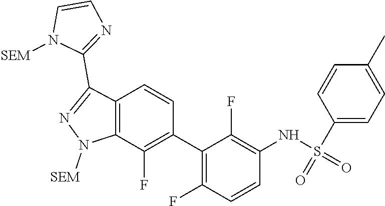 [SMILES: Cc1ccc(S(=O)(=O)Nc2ccc(F)c(-c3ccc4c(-c5nccn5COCC[Si](C)(C)C)nn(COCC[Si](C)(C)C)c4c3F)c2F)cc1]

A mixture of 2,4-difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)aniline (150 mg, 0.25 mmol), pyridine (4.0 mL), and 4-methylbenzene-1-sulfonyl chloride (58 mg, 0.31 mmol) was stirred overnight at room temperature then concentrated. The resulting residue was partitioned between ethyl acetate and 0.5 M HCl. The organic layer was dried (Na₂SO₄) and concentrated to afford N-(2,4-difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)phenyl)-4-methylbenzenesulfonamide (150 mg) which was used in the next step without further purification.

### Part IX—Synthesis of N-(2,4-Difluoro-3-(7-fluoro-3-(1H-imidazol-2-yl)-1H-indazol-6-yl)phenyl)-4-methylbenzenesulfonamide

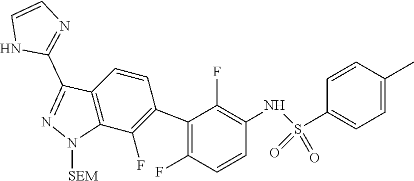 [SMILES: Cc1ccc(S(=O)(=O)Nc2ccc(F)c(-c3ccc4c(-c5ncc[nH]5)nn(COCC[Si](C)(C)C)c4c3F)c2F)cc1]

A mixture of N-(2,4-difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)phenyl)-4-methylbenzenesulfonamide (150 mg, 0.20 mmol), dichloromethane (6.0 mL), and trifluoroacetic acid (3.0 mL) was stirred for three hours at room temperature then concentrated. Methanol (6.0 mL) and potassium carbonate (100 mg) were added and the mixture was stirred for an additional three hours at room temperature. The mixture was extracted three times with ethyl acetate. The resulting extracts were combined and washed with brine, dried (Na₂SO₄) and concentrated. The resulting residue was purified by Prep-HPLC eluting with a gradient of 30-60% acetonitrile in aqueous ammonium bicarbonate to afford N-(2,4-difluoro-3-(7-fluoro-3-(1H-imidazol-2-yl)-1H-indazol-6-yl)phenyl)-4-methylbenzenesulfonamide (41 mg, 42%) as a white solid. ¹H-NMR (400 MHz, CD₃OD-d₄) δ 8.12 (d, J=8.4 Hz, 1H), 7.65 (d, J=8.4 Hz, 2H), 7.58 (m, 1H), 7.35 (d, J=8.4 Hz, 2H), 7.25 (s, 2H), 7.11 (t, J=5.6 Hz, 1H), 6.96 (m, 1H), 2.44 (s, 3H). (ES, m/z): (M+H)⁺484.

### Example 2—Synthesis of N-(2,4-difluoro-3-(7-fluoro-3-(1H-imidazol-2-yl)-1H-indazol-6-yl)phenyl)-2-methylpyridine-3-sulfonamide

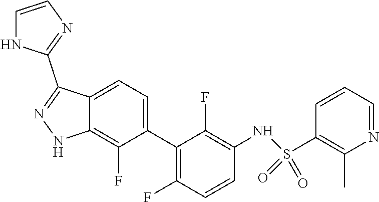 [SMILES: Cc1ncccc1S(=O)(=O)Nc1ccc(F)c(-c2ccc3c(-c4ncc[nH]4)n[nH]c3c2F)c1F]

### Part I—Synthesis of 3-(Benzylthio)-2-methylpyridine

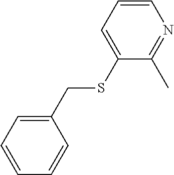

A mixture of 3-bromo-2-methylpyridine (2 g, 11.6 mmol), toluene (15 mL), benzylthiol (1.44 g, 11.6 mmol), diisopropylethyl amine (3.0 g, 23 mmol), 4,5-bis(diphenylphosphino)-9,9-dimethylxanthene (672 mg, 1.16 mmol), and tris(dibenzylideneacetone)dipalladium(0) (530 mg, 0.579 mmol) was stirred for two hours at 110° C. The mixture was cooled and filtered through Celite, then washed with ethyl acetate. The combined filtrate was concentrated and the resulting residue was purified by MPLC eluting with a gradient of 0-10% ethyl acetate in petroleum ether to afford 3-(benzylthio)-2-methylpyridine (1.3 g, 52%) as a light yellow oil.

### Part II—Synthesis of 2-Methylpyridine-3-sulfonyl chloride

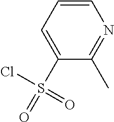

A mixture of 3-(benzylthio)-2-methylpyridine (600 mg, 2.8 mmol), acetic acid (9 mL), water (3 mL), and N-chlorosuccinimide (1.49 g, 11.2 mmol) was stirred for two hours at room temperature. Water was added and the mixture was extracted three times with ethyl acetate. The combined organic layers were concentrated and the resulting residue was triturated with hexane (5 mL) to afford 2-methylpyridine-3-sulfonyl chloride (420 mg, 79%) as a colorless oil.

### Part III—Synthesis of N-(2,4-Difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)phenyl)-2-methylpyridine-3-sulfonamide

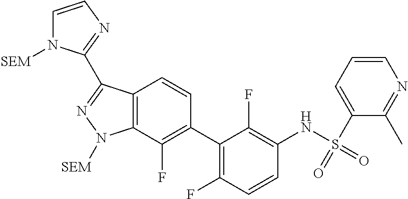 [SMILES: Cc1ncccc1S(=O)(=O)Nc1ccc(F)c(-c2ccc3c(-c4nccn4COCC[Si](C)(C)C)nn(COCC[Si](C)(C)C)c3c2F)c1F]

A mixture of 2,4-difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)aniline (200 mg, 0.34 mmol), pyridine (5 mL), and 2-methylpyridine-3-sulfonyl chloride (130 mg, 0.68 mmol) was stirred overnight at room temperature then concentrated. The resulting residue was purified by MPLC eluting with a gradient of 10-20% ethyl acetate in petroleum ether to afford N-(2,4-difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)phenyl)-2-methylpyridine-3-sulfonamide (150 mg, 59%).

### Part IV—Synthesis of N-(2,4-Difluoro-3-(7-fluoro-3-(1H-imidazol-2-yl)-1H-indazol-6-yl)phenyl)-2-methylpyridine-3-sulfonamide

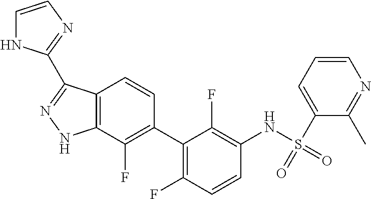 [SMILES: Cc1ncccc1S(=O)(=O)Nc1ccc(F)c(-c2ccc3c(-c4ncc[nH]4)n[nH]c3c2F)c1F]

A mixture of N-(2,4-difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)phenyl)-2-methylpyridine-3-sulfonamide (200 mg, 0.34 mmol) in trifluoroacetic acid (3 mL) was stirred for two hours at room temperature then concentrated. A solution of aqueous ammonia in methanol (10 mL) was added and stirred for thirty minutes, then concentrated. The resulting residue was purified by Prep-HPLC eluting with a gradient of 5-38% acetonitrile in 0.05% trifluoroacetic acid in water to afford N-(2,4-difluoro-3-(7-fluoro-3-(1H-imidazol-2-yl)-1H-indazol-6-yl)phenyl)-2-methylpyridine-3-sulfonamide (115 mg, 72%) as a white solid. ¹H-NMR (400 MHz, CD₃OD-d₄) δ 8.60 (dd, J=1.6 Hz, 4.8 Hz, 1H), 8.17 (d, J=6.8 Hz, 1H), 8.00 (d, J=8.4 Hz, 1H), 7.74 (s, 2H), 7.62-7.56 (m, 1H), 7.40-7.37 (m, 1H), 7.22-7.13 (m, 2H), 2.86 (s, 3H). (ES, m/z): (M+H)⁺ 485.

### Example 3—Synthesis of N-(2,4-Difluoro-3-(7-fluoro-3-(1H-imidazol-2-yl)-1H-indazol-6-yl)phenyl)-3-(hydroxymethyl)benzenesulfonamide

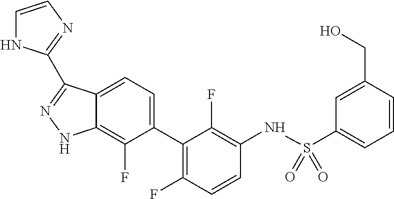 [SMILES: O=S(=O)(Nc1ccc(F)c(-c2ccc3c(-c4ncc[nH]4)n[nH]c3c2F)c1F)c1cccc(CO)c1]

### Part I—Synthesis of Methyl 3-(N-(2,4-Difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)phenyl)sulfamoyl)benzoate

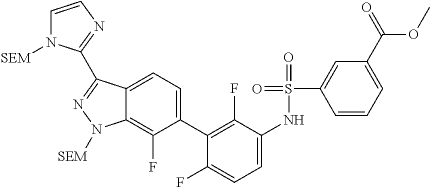 [SMILES: COC(=O)c1cccc(S(=O)(=O)Nc2ccc(F)c(-c3ccc4c(-c5nccn5COCC[Si](C)(C)C)nn(COCC[Si](C)(C)C)c4c3F)c2F)c1]

A mixture of 2,4-difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)aniline (500 mg, 0.85 mmol), pyridine (10 mL), and methyl 3-(chlorosulfonyl)benzoate (200 mg, 0.85 mmol) was stirred overnight at room temperature and concentrated. The resulting residue was purified via MPLC eluting with 25% ethyl acetate in petroleum ether to afford methyl 3-(N-(2,4-difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)phenyl)sulfamoyl)benzoate (695 mg, 100%).

### Part II—Synthesis of Methyl 3-(N-(2,4-Difluoro-3-(7-fluoro-3-(1H-imidazol-2-yl)-1H-indazol-6-yl)phenyl)sulfamoyl)benzoate

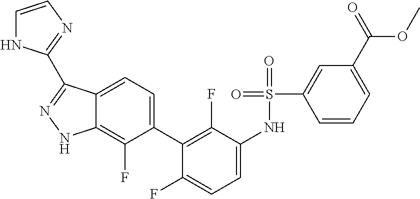 [SMILES: COC(=O)c1cccc(S(=O)(=O)Nc2ccc(F)c(-c3ccc4c(-c5ncc[nH]5)n[nH]c4c3F)c2F)c1]

A mixture of methyl 3-(N-(2,4-difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)phenyl)sulfamoyl)benzoate (695 mg, 0.88 mmol), dichloromethane (2 mL) and trifluoroacetic acid (6 mL) was stirred for three hours at room temperature then concentrated. A solution of 7M ammonia in methanol (5 mL) was added and stirred for three hours then concentrated. The resulting residue was purified via MPLC eluting with 50% ethyl acetate in hexanes to afford methyl 3-(N-(2,4-difluoro-3-(7-fluoro-3-(1H-imidazol-2-yl)-1H-indazol-6-yl)phenyl)sulfamoyl)benzoate (230 mg, 49%).

### Part III—Synthesis of N-(2,4-Difluoro-3-(7-fluoro-3-(1H-imidazol-2-yl)-1H-indazol-6-yl)phenyl)-3-(hydroxymethyl)benzenesulfonamide

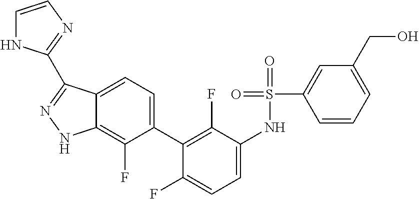 [SMILES: O=S(=O)(Nc1ccc(F)c(-c2ccc3c(-c4ncc[nH]4)n[nH]c3c2F)c1F)c1cccc(CO)c1]

Lithium aluminum hydride (39 mg, 1.0 mmol) was added to a solution of methyl 3-(N-(2,4-difluoro-3-(7-fluoro-3-(1H-imidazol-2-yl)-1H-indazol-6-yl)phenyl)sulfamoyl)benzoate (218 mg, 0.413 mmol), in THE (10 mL). The mixture was stirred for two hours at room temperature, then quenched by the addition of water. The mixture was extracted three times with ethyl acetate. The combined organic layers were dried (Na₂SO₄) and concentrated. The resulting residue was purified by Prep-HPLC eluting with a gradient of 10-40% acetonitrile in aqueous ammonium bicarbonate to afford N-(2,4-difluoro-3-(7-fluoro-3-(1H-imidazol-2-yl)-1H-indazol-6-yl)phenyl)-3-(hydroxymethyl)benzenesulfonamide (72 mg, 35%) of N-(2,4-difluoro-3-(7-fluoro-3-(1H-imidazol-2-yl)-1H-indazol-6-yl)phenyl)-3-(hydroxymethyl)benzenesulfonamide as a solid. ¹H-NMR (400 MHz, CD₃OD-d₄) δ 8.11 (d, J=8.4 Hz, 1H), 7.82 (s, 1H), 7.67 (d, J=7.7 Hz, 1H), 7.63-7.43 (m, 3H), 7.24 (s, 2H), 7.16-6.90 (m, 2H), 4.66 (s, 2H). (ES, m/z): (M+H)⁺ 500.

### Example 4—Synthesis of Methyl 6-(3-((5-Chloro-2-methoxypyridine)-3-sulfonamido)-2,6-difluorophenyl)-7-fluoro-1H-indazole-3-carboxylate

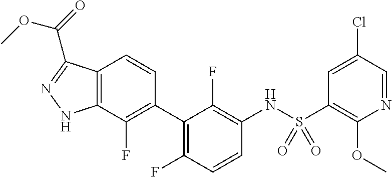

### Part I—Synthesis of 6-Bromo-7-fluoroindoline-2,3-dione

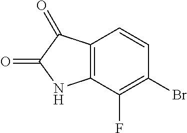 [SMILES: O=C1Nc2c(ccc(Br)c2F)C1=O]

A mixture of 3-bromo-2-fluoroaniline (5.0 g, 26.3 mmol), 2,2,2-trichloroethane-1,1-diol (4.35 g, 26.3 mmol), 1M aqueous sulfuric acid (20 mL), water (75 mL), sodium sulfate (50.0 g, 352 mmol), and hydroxylamine hydrochloride (5.50 g, 79.7 mmol) was stirred for thirty minutes at 130° C. The mixture was then cooled to 80° C., and filtered. The solids were washed with water. The filtrate was concentrated. The resulting residue was diluted with concentrated sulfuric acid. The resulting solution was stirred for an additional hour at 70° C. The reaction was then cooled and a mixture of ice and water was added. The mixture was extracted three times with ethyl acetate. The combined organic layers were washed with brine and concentrated. The resulting residue was purified via MPLC eluting with a gradient of 0-25% ethyl acetate in petroleum ether to afford 6-bromo-7-fluoroindoline-2,3-dione (3.0 g, 47%) as a yellow solid.

### Part II—Synthesis of 6-Bromo-7-fluoro-1H-indazole-3-carboxylic acid

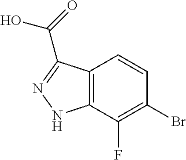 [SMILES: O=C(O)c1n[nH]c2c(F)c(Br)ccc12]

A mixture of 6-bromo-7-fluoroindoline-2,3-dione (1.0 g, 4.1 mmol), sodium hydroxide (197 mg) and water (5 mL) was stirred for thirty minutes at 50° C. and an hour at room temperature. A solution of sodium nitrite (0.34 g, 4.9 mmol) in water (2.0 mL) was added dropwise with stirring at 0° C. The mixture was then added dropwise a solution of sulfuric acid (0.5 mL) in water (20 mL) at 0° C. and stirred for an additional 30 minutes. A solution of SnCl₂.2H₂O (2.3 g, 10.2 mmol) in concentrated HCl (10.0 mL) was then added dropwise with stirring at 0° C. The resulting mixture was stirred for an additional hour at 0° C., and then quenched by the addition of ice water. The mixture was extracted three times with ethyl acetate. The combined organic layers were washed with brine and concentrated. The resulting residue was purified via MPLC eluting with a gradient of 0-10% methanol in dichloromethane to afford 6-bromo-7-fluoro-1H-indazole-3-carboxylic acid (150 mg, 14%) as a light yellow solid.

### Part III—Synthesis of Methyl 6-bromo-7-fluoro-1H-indazole-3-carboxylate

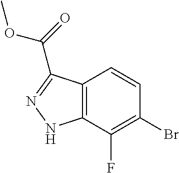

A mixture of 6-bromo-7-fluoro-1H-indazole-3-carboxylic acid (600 mg, 2.3 mmol), methanol (20 mL), and sulfuric acid (0.4 mL) was stirred overnight at 70° C. The resulting solution was concentrated, diluted with brine, and extracted with ethyl acetate. The organic layer was dried (Na₂SO₄) and concentrated. The resulting residue was purified by MPLC eluting with a gradient of 0-25% ethyl acetate in petroleum ether to afford methyl 6-bromo-7-fluoro-1H-indazole-3-carboxylate (300 mg, 47%) as a light yellow oil.

### Part IV—Synthesis of Methyl 6-Bromo-7-fluoro-1-((2-(trimethylsilyl)ethoxy)methyl)-1H-indazole-3-carboxylate

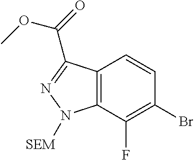 [SMILES: COC(=O)c1nn(COCC[Si](C)(C)C)c2c(F)c(Br)ccc12]

A mixture of 60% sodium hydride in mineral oil (53 mg) was added portionwise to a solution of methyl 6-bromo-7-fluoro-1H-indazole-3-carboxylate (300 mg, 1.1 mmol) in THE (20 mL) at 0° C. After stirring an additional thirty minutes at 0° C., 2-(trimethylsilyl)ethoxymethyl chloride (220 mg, 1.32 mmol) was added dropwise. The mixture was allowed to stir for an additional two hours at room temperature. Water was added and the mixture was extracted three times with ethyl acetate. The combined organic layers were washed with brine, dried (Na₂SO₄), and concentrated. The resulting residue was purified by MPLC eluting with a gradient of 0-20% ethyl acetate in petroleum ether to afford methyl 6-bromo-7-fluoro-1-((2-(trimethylsilyl)ethoxy)methyl)-1H-indazole-3-carboxylate (300 mg, 68%) as a solid.

### Part V—Synthesis of Methyl 7-fluoro-6-(4,4,5,5-tetramethyl-1,3,2-dioxaborolan-2-yl)-1-((2-(trimethylsilyl)ethoxy)methyl)-1H-indazole-3-carboxylate

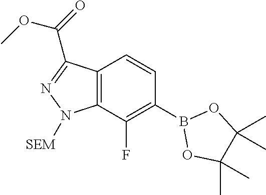

A mixture of methyl 6-bromo-7-fluoro-1-((2-(trimethylsilyl)ethoxy)methyl)-1H-indazole-3-carboxylate (300 mg, 0.744 mmol), dioxane (20 mL), 4,4,5,5-tetramethyl-2-(4,4,5,5-tetramethyl-1,3,2-dioxaborolan-2-yl)-1,3,2-dioxaborolane (283 mg, 1.12 mmol), [1,1′-bis(diphenylphosphino)ferrocene]dichloropalladium(II) (54 mg, 0.074 mmol), and potassium acetate (219 mg, 2.2 mmol) was stirred overnight at 105° C. The mixture was concentrated and the resulting residue was purified by MPLC eluting with a gradient of 0-20% ethyl acetate in petroleum ether to afford methyl 7-fluoro-6-(4,4,5,5-tetramethyl-1,3,2-dioxaborolan-2-yl)-1-((2-(trimethylsilyl)ethoxy)methyl)-1H-indazole-3-carboxylate (250 mg, 75%) as a solid.

### Part VI—Synthesis of Methyl 6-(2,6-Difluoro-3-nitrophenyl)-7-fluoro-1-((2-(trimethylsilyl)ethoxy)methyl)-1H-indazole-3-carboxylate

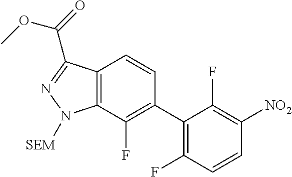 [SMILES: COC(=O)c1nn(COCC[Si](C)(C)C)c2c(F)c(-c3c(F)ccc([N+](=O)[O-])c3F)ccc12]

A mixture of methyl 7-fluoro-6-(4,4,5,5-tetramethyl-1,3,2-dioxaborolan-2-yl)-1-((2-(trimethylsilyl)ethoxy)methyl)-1H-indazole-3-carboxylate (250 mg, 0.56 mmol), 2-bromo-1,3-difluoro-4-nitrobenzene (158 mg, 0.67 mmol), tetrakis(triphenylphosphine)palladium(0) (64 mg, 0.056 mmol), toluene (9 mL), ethanol (3 mL), water (3 mL), and potassium carbonate (230 mg, 1.67 mmol) was stirred for three hours at 110° C. The mixture was concentrated and the resulting residue was purified by MPLC eluting with a gradient of 0-20% ethyl acetate in petroleum ether to afford methyl 6-(2,6-difluoro-3-nitrophenyl)-7-fluoro-1-((2-(trimethylsilyl)ethoxy)methyl)-1H-indazole-3-carboxylate (200 mg, 74%) as a solid.

### Part VII—Synthesis of Methyl 6-(3-Amino-2,6-difluorophenyl)-7-fluoro-1-((2-(trimethylsilyl)ethoxy)methyl)-1H-indazole-3-carboxylate

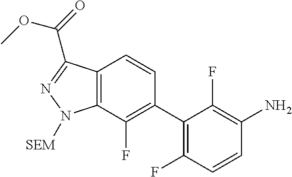 [SMILES: COC(=O)c1nn(COCC[Si](C)(C)C)c2c(F)c(-c3c(F)ccc(N)c3F)ccc12]

A mixture of methyl 6-(2,6-difluoro-3-nitrophenyl)-7-fluoro-1-((2-(trimethylsilyl)ethoxy)methyl)-1H-indazole-3-carboxylate (200 mg, 0.415 mmol), ethyl acetate (20 mL), and 10% palladium on carbon (200 mg) was stirred for three hours at room temperature under an atmosphere of hydrogen. The mixture was filtered through Celite, washed with ethyl acetate and the combined filtrate was concentrated. The resulting residue was purified by MPLC eluting with a gradient of 0-33% ethyl acetate in petroleum ether to afford methyl 6-(3-amino-2,6-difluorophenyl)-7-fluoro-1-((2-(trimethylsilyl)ethoxy)methyl)-1H-indazole-3-carboxylate (150 mg, 80%) as an oil.

### Part VIII—Synthesis of Methyl 6-(3-((5-Chloro-2-methoxypyridine)-3-sulfonamido)-2,6-difluorophenyl)-7-fluoro-1-((2-(trimethylsilyl)ethoxy)methyl)-1H-indazole-3-carboxylate

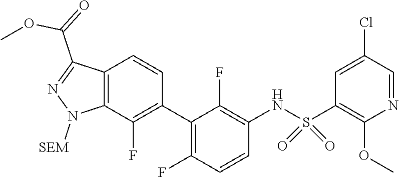

5-Chloro-2-methoxypyridine-3-sulfonyl chloride (241 mg, 1.0 mmol) was slowly added to a solution of methyl 6-(3-amino-2,6-difluorophenyl)-7-fluoro-1-((2-(trimethylsilyl)ethoxy)methyl)-1H-indazole-3-carboxylate (150 mg, 0.33 mmol) in pyridine (5 mL) at 0° C. The mixture was stirred for an additional six hours at room temperature, concentrated, and partitioned between ethyl acetate and 0.5 M HCl. The organic layer was concentrated and the resulting residue was purified by MPLC eluting with a gradient of 0-25% ethyl acetate in petroleum ether to afford methyl 6-(3-((5-chloro-2-methoxypyridine)-3-sulfonamido)-2,6-difluorophenyl)-7-fluoro-1-((2-(trimethylsilyl)ethoxy)methyl)-1H-indazole-3-carboxylate (150 mg, 69%) as a solid.

### Part IX—Synthesis of Methyl 6-(3-((5-Chloro-2-methoxypyridine)-3-sulfonamido)-2,6-difluorophenyl)-7-fluoro-1H-indazole-3-carboxylate

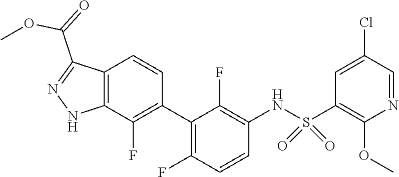

A solution of methyl 6-(3-((5-chloro-2-methoxypyridine)-3-sulfonamido)-2,6-difluorophenyl)-7-fluoro-1-((2-(trimethylsilyl)ethoxy)methyl)-1H-indazole-3-carboxylate (20 mg, 0.030 mmol), dichloromethane (6 mL), and trifluoroacetic acid (3 mL) was stirred for three hours at room temperature. The resulting mixture was concentrated, and the resulting residue was purified via Prep-HPLC eluting with a gradient of 30-55% acetonitrile in aqueous ammonium bicarbonate to afford methyl 6-(3-((5-chloro-2-methoxypyridine)-3-sulfonamido)-2,6-difluorophenyl)-7-fluoro-1H-indazole-3-carboxylate (6.4 mg, 40%) as a white solid. ¹H-NMR (400 MHz, CD₃OD-d₄) δ 8.36 (d, J=7.2 Hz, 1H), 8.11 (s, 1H), 8.00 (d, J=7.2 Hz, 1H), 7.70-7.50 (m, 1H), 7.25-7.10 (m, 2H), 4.05 (s, 3H), 4.00 (s, 3H). (ES, m/z): (M+H)⁺ 527.

### Example 5—Synthesis of 6-(3-((5-chloro-2-methoxypyridine)-3-sulfonamido)-2,6-difluorophenyl)-7-fluoro-N-methyl-1H-indazole-3-carboxamide

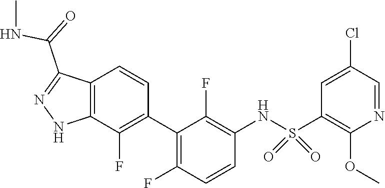 [SMILES: CNC(=O)c1n[nH]c2c(F)c(-c3c(F)ccc(NS(=O)(=O)c4cc(Cl)cnc4OC)c3F)ccc12]

Methyl 6-(3-((5-chloro-2-methoxypyridine)-3-sulfonamido)-2,6-difluorophenyl)-7-fluoro-1H-indazole-3-carboxylate (80 mg, 0.15 mmol) was added to a solution of 25% methylamine solution in ethanol (20 mL) and stirred overnight at 60° C. The mixture was concentrated, and the resulting residue was purified by Prep-HPLC with a gradient of 15-35% acetonitrile in aqueous ammonium bicarbonate to afford 6-(3-((5-chloro-2-methoxypyridine)-3-sulfonamido)-2,6-difluorophenyl)-7-fluoro-N-methyl-1H-indazole-3-carboxamide (33 mg, 56%) as a white solid. ¹H NMR (300 MHz, CD₃OD-d₄) δ 8.36 (d, J=7.2 Hz, 1H), 8.15-8.10 (m, 2H), 7.65-7.50 (m, 1H), 7.15-7.05 (m, 2H), 4.00 (s, 3H), 3.00 (s, 3H). (ES, m/z): (M+H)⁺ 526.

### Example 6—Synthesis of 5-Chloro-N-(3,5-difluoro-4-(7-fluoro-3-(1H-imidazol-2-yl)-1H-indazol-6-yl)pyridin-2-yl)-2-methoxypyridine-3-sulfonamide

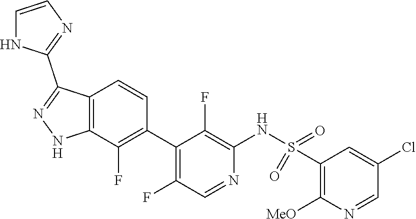 [SMILES: COc1ncc(Cl)cc1S(=O)(=O)Nc1ncc(F)c(-c2ccc3c(-c4ncc[nH]4)n[nH]c3c2F)c1F]

### Part I—Synthesis of 5-Chloro-N-(3,5-difluoro-4-iodopyridin-2-yl)-2-methoxypyridine-3-sulfonamide

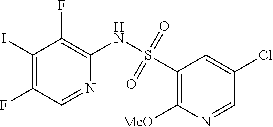 [SMILES: COc1ncc(Cl)cc1S(=O)(=O)Nc1ncc(F)c(I)c1F]

A mixture of 3,5-difluoro-4-iodopyridin-2-amine (1.86 g, 7.27 mmol), 5-chloro-2-methoxypyridine-3-sulfonyl chloride (2.29 g, 9.45 mmol), and pyridine (24 mL) was stirred at 80° C. overnight. The mixture was concentrated and the resulting residue was partitioned between 1M aqueous citric acid solution and ethyl acetate. The organic layer was washed with brine, dried (Na₂SO₄), and concentrated. The resulting residue was purified via MPLC eluting with a gradient of 10-100% ethyl acetate in hexanes to afford 5-chloro-N-(3,5-difluoro-4-iodopyridin-2-yl)-2-methoxypyridine-3-sulfonamide (800 mg, 24%).

### Part II—Synthesis of 5-Chloro-N-(3,5-difluoro-4-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)pyridin-2-yl)-2-methoxypyridine-3-sulfonamide

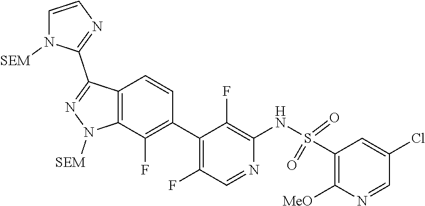 [SMILES: COc1ncc(Cl)cc1S(=O)(=O)Nc1ncc(F)c(-c2ccc3c(-c4nccn4COCC[Si](C)(C)C)nn(COCC[Si](C)(C)C)c3c2F)c1F]

A mixture of 5-chloro-N-(3,5-difluoro-4-iodopyridin-2-yl)-2-methoxypyridine-3-sulfonamide (140 mg, 0.303 mmol), 7-fluoro-6-(4,4,5,5-tetramethyl-1,3,2-dioxaborolan-2-yl)-1-((2-(trimethylsilyl)ethoxy)methyl)-3-(1-((2-(trimethylsilyl)ethoxy)methyl)-1H-imidazol-2-yl)-1H-indazole (179 mg, 0.303 mmol), potassium carbonate (84 mg, 0.607 mmol), toluene (2.8 mL), ethanol (0.7 mL), and water (0.7 mL), was degassed with a stream of nitrogen. Tetrakis(triphenylphospine)palladium (0) (35 mg, 0.03 mmol) was then added, and the reaction was heated to 100° C. overnight. The mixture was partitioned between water and ethyl acetate. The organic layer was then dried (Na₂SO₄), and concentrated. The resulting residue was purified by MPLC eluting with a gradient of 0-10% methanol in dichloromethane to afford 5-chloro-N-(3,5-difluoro-4-(7-fluoro-1-((2-(trimethylsilyl)ethoxy)-methyl)-3-(1-((2-(trimethylsilyl)ethoxy)methyl)-1H-imidazol-2-yl)-1H-indazol-6-yl)pyridin-2-yl)-2-methoxypyridine-3-sulfonamide (100 mg, 41%).

### Part III—Synthesis of 5-Chloro-N-(3,5-difluoro-4-(7-fluoro-3-(1H-imidazol-2-yl)-1H-indazol-6-yl)pyridin-2-yl)-2-methoxypyridine-3-sulfonamide

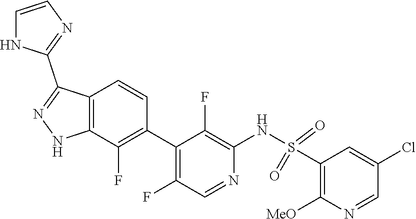 [SMILES: COc1ncc(Cl)cc1S(=O)(=O)Nc1ncc(F)c(-c2ccc3c(-c4ncc[nH]4)n[nH]c3c2F)c1F]

A solution of 5-chloro-N-(3,5-difluoro-4-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)pyridin-2-yl)-2-methoxypyridine-3-sulfonamide (100 mg, 0.125 mmol) dissolved in dichloromethane (5 mL) and trifluoroacetic acid (3 mL) was stirred for three hours at room temperature then concentrated. The resulting residue was dissolved in 10% ammonium hydroxide in methanol and the solution was stirred at room temperature for one hour then concentrated. The resulting residue was purified by MPLC eluting with a gradient of 0-10% methanol in dichloromethane to afford 5-chloro-N-(3,5-difluoro-4-(7-fluoro-3-(1H-imidazol-2-yl)-1H-indazol-6-yl)pyridin-2-yl)-2-methoxypyridine-3-sulfonamide (34 mg, 51%): ¹H NMR (400 MHz, DMSO-d₆) δ 8.52 (1H, br s), 8.16-8.40 (3H, m) 7.26 (3H, br s) 3.94 (3H, br s). (ES, m/z): (M+H)⁺ 536.

### Example 7—Synthesis of N-(3-(3-Amino-7-fluoro-1H-indazol-6-yl)-2,4-difluorophenyl)-5-chloro-2-methoxypyridine-3-sulfonamide

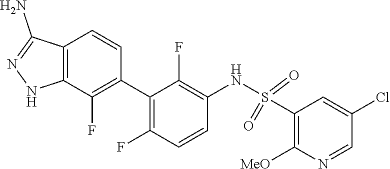 [SMILES: COc1ncc(Cl)cc1S(=O)(=O)Nc1ccc(F)c(-c2ccc3c(N)n[nH]c3c2F)c1F]

### Part I—Synthesis of 6-Bromo-7-fluoro-1H-indazol-3-amine

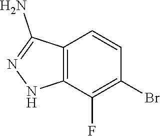 [SMILES: Nc1n[nH]c2c(F)c(Br)ccc12]

A mixture of 4-bromo-2,3-difluorobenzonitrile (1.00 g, 4.59 mmol), hydrazine (1.0 mL), and n-BuOH (10 mL) was stirred overnight at 120° C. The reaction mixture was cooled and diluted with water (100 mL). The mixture was extracted three times with ethyl acetate, and the combined organic layers were dried (Na₂SO₄) and concentrated to afford 6-bromo-7-fluoro-1H-indazol-3-amine (1.0 g, 95%) as a solid.

### Part II—Synthesis of 7-Fluoro-6-(4,4,5,5-tetramethyl-1,3,2-dioxaborolan-2-yl)-1H-indazol-3-amine

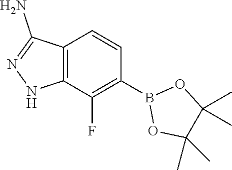 [SMILES: CC1(C)OB(c2ccc3c(N)n[nH]c3c2F)OC1(C)C]

A mixture of 6-bromo-7-fluoro-1H-indazol-3-amine (900 mg, 3.91 mmol), 1,4-dioxane (20 mL), 4,4,5,5-tetramethyl-2-(4,4,5,5-tetramethyl-1,3,2-dioxaborolan-2-yl)-1,3,2-dioxaborolane (1.49 g, 5.87 mmol), potassium acetate (1.15 g, 11.7 mmol), and [1, l-bis(diphenylphosphino)ferrocene]dichloropalladium(II) (287 mg, 0.39 mmol) was stirred for four hours at 110° C. The mixture was concentrated and the resulting residue was purified by MPLC eluting with 25% ethyl acetate in petroleum ether to afford 7-fluoro-6-(4,4,5,5-tetramethyl-1,3,2-dioxaborolan-2-yl)-1H-indazol-3-amine (842 mg, 78%).

### Part III—Synthesis of N-(3-(3-amino-7-fluoro-1H-indazol-6-yl)-2,4-difluorophenyl)-5-chloro-2-methoxypyridine-3-sulfonamide

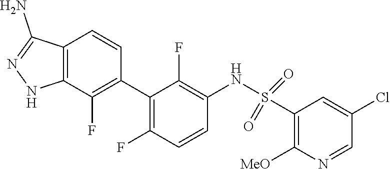 [SMILES: COc1ncc(Cl)cc1S(=O)(=O)Nc1ccc(F)c(-c2ccc3c(N)n[nH]c3c2F)c1F]

A mixture of 7-fluoro-6-(4,4,5,5-tetramethyl-1,3,2-dioxaborolan-2-yl)-1H-indazol-3-amine (605 mg, 2.18 mmol), N-(3-bromo-2,4-difluorophenyl)-5-chloro-2-methoxypyridine-3-sulfonamide (941 mg, 2.28 mmol), 1,4-dioxane (16 mL), potassium carbonate (904, 6.54 mmol), [1,1′-bis(diphenylphosphino)ferrocene]dichloropalladium(II) (160 mg, 0.22 mmol), and water (4 mL) was stirred for two hours at 100° C. The mixture was concentrated. The resulting residue was purified by MPLC eluting with ethyl acetate to afford N-(3-(3-amino-7-fluoro-1H-indazol-6-yl)-2,4-difluorophenyl)-5-chloro-2-methoxypyridine-3-sulfonamide (30.9 mg, 3%). ¹H NMR (400 MHz, CD₃OD-d₄) δ 8.35 (d, J=2.6 Hz, 1H), 8.07 (d, J=2.6 Hz, 1H), 7.64-7.49 (m, 2H), 7.11 (td, J=8.9, 1.8 Hz, 1H), 6.85 (dd, J=8.3, 5.5 Hz, 1H), 4.00 (s, 3H). (ES, m/z): (M+H)⁺ 484.

### Example 8—Synthesis of N-(6-(3-((5-Chloro-2-methoxypyridine)-3-sulfonamido)-2,6-difluorophenyl)-7-fluoro-1H-indazol-3-yl)acetamide

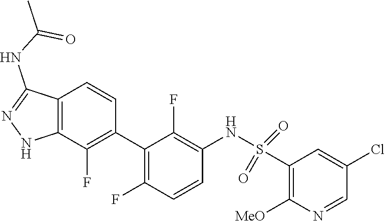 [SMILES: COc1ncc(Cl)cc1S(=O)(=O)Nc1ccc(F)c(-c2ccc3c(NC(C)=O)n[nH]c3c2F)c1F]

A solution of N-(3-(3-amino-7-fluoro-1H-indazol-6-yl)-2,4-difluorophenyl)-5-chloro-2-methoxypyridine-3-sulfonamide (150 mg, 0.310 mmol), THE (15 mL), and acetic anhydride (0.5 mL) was stirred overnight at room temperature. The mixture was diluted with water and extracted three times with ethyl acetate. The combined organic layers were dried (Na₂SO₄) and concentrated. The resulting residue was purified by MPLC eluting with 25% ethyl acetate in hexane to afford N-(6-(3-((5-chloro-2-methoxypyridine)-3-sulfonamido)-2,6-difluorophenyl)-7-fluoro-1H-indazol-3-yl)acetamide (6.9 mg, 4%) as a solid. ¹H NMR (400 MHz, CD₃OD-d₄) δ 8.35 (d, J=2.6 Hz, 1H), 8.07 (d, J=2.6 Hz, 1H), 7.64-7.49 (m, 2H), 7.11 (td, J=8.9, 1.8 Hz, 1H), 6.85 (dd, J=8.3, 5.5 Hz, 1H), 4.00 (s, 3H), 2.30 (s, 3H). (ES, m/z): (M+H)⁺ 526.

### Example 9—Preparation of Additional (Aza)Indazolyl-Aryl Sulfonamide and Related Compounds

Compounds in Table 3 were prepared based on experimental procedures described in Examples 1-8 and the Detailed Description. Additional physical characterization data (e.g., ¹H NMR data) for exemplary compounds are provided in Table 4.

TABLE 3

| Compd | | | Observed |
|---|---|---|---|
| No. | Structure | Name | m/z |
| 2A | 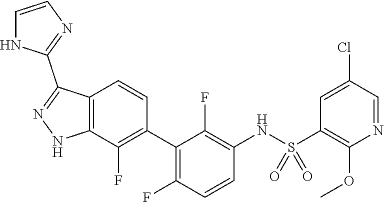 [SMILES: COc1ncc(Cl)cc1S(=O)(=O)Nc1ccc(F)c(-c2ccc3c(-c4ncc[nH]4)n[nH]c3c2F)c1F] | 5-chloro-N-(2,4-difluoro-3-(7- fluoro-3-(1H-imidazol-2-yl)- 1H-indazol-6-yl)phenyl)-2- methoxy-pyridine-3- sulfonamide | 535 (M + H)⁺ |
| 2B | 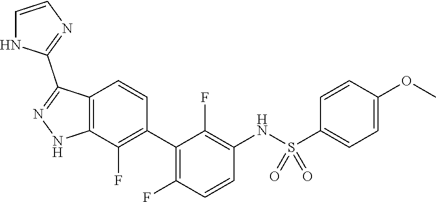 [SMILES: COc1ccc(S(=O)(=O)Nc2ccc(F)c(-c3ccc4c(-c5ncc[nH]5)n[nH]c4c3F)c2F)cc1] | N-(2,4-difluoro-3-(7-fluoro-3- (1H-imidazol-2-yl)-1H- indazol-6-yl)phenyl)-4- methoxybenzene-sulfonamide | 500 (M + H)⁺ |
| 2C | 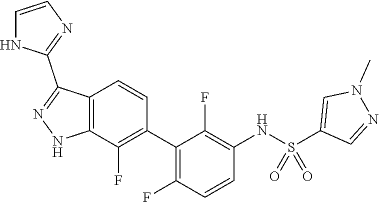 [SMILES: Cn1cc(S(=O)(=O)Nc2ccc(F)c(-c3ccc4c(-c5ncc[nH]5)n[nH]c4c3F)c2F)cn1] | N-(2,4-difluoro-3-(7-fluoro-3- (1H-imidazol-2-yl)-1H- indazol-6-yl)phenyl)-1- methyl-1H-pyrazole-4- sulfonamide | 474 (M + H)⁺ |
| 2D | 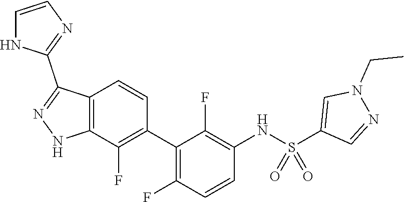 [SMILES: CCn1cc(S(=O)(=O)Nc2ccc(F)c(-c3ccc4c(-c5ncc[nH]5)n[nH]c4c3F)c2F)cn1] | N-(2,4-difluoro-3-(7-fluoro-3- (1H-imidazol-2-yl)-1H- indazol-6-yl)phenyl)-1-ethyl- 1H-pyrazole-4-sulfonamide | 488 (M + H)⁺ |
| 2E | 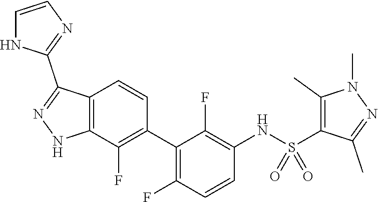 [SMILES: Cc1nn(C)c(C)c1S(=O)(=O)Nc1ccc(F)c(-c2ccc3c(-c4ncc[nH]4)n[nH]c3c2F)c1F] | N-(2,4-difluoro-3-(7-fluoro-3- (1H-imidazol-2-yl)-1H- indazol-6-yl)phenyl)-1,3,5- trimethyl-1H-pyrazole-4- sulfonamide | 502 (M + H)⁺ |
| 2F | 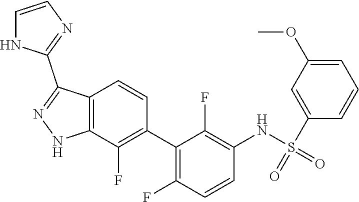 [SMILES: COc1cccc(S(=O)(=O)Nc2ccc(F)c(-c3ccc4c(-c5ncc[nH]5)n[nH]c4c3F)c2F)c1] | N-(2,4-difluoro-3-(7-fluoro-3- (1H-imidazol-2-yl)-1H- indazol-6-yl)phenyl)-3- methoxy-benzene- sulfonamide | 500 (M + H)⁺ |
| 2G | 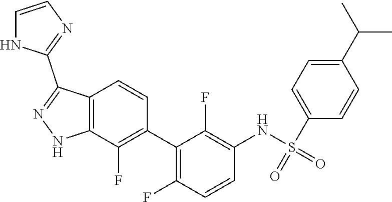 [SMILES: CC(C)c1ccc(S(=O)(=O)Nc2ccc(F)c(-c3ccc4c(-c5ncc[nH]5)n[nH]c4c3F)c2F)cc1] | N-(2,4-difluoro-3-(7-fluoro-3- (1H-imidazol-2-yl)-1H- indazol-6-yl)phenyl)-4- isopropyl- benzenesulfonamide | 512 (M + H)⁺ |
| 2H | 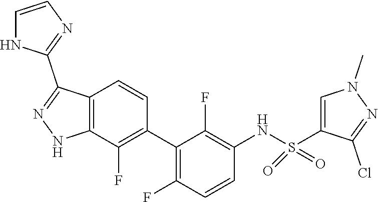 [SMILES: Cn1cc(S(=O)(=O)Nc2ccc(F)c(-c3ccc4c(-c5ncc[nH]5)n[nH]c4c3F)c2F)c(Cl)n1] | 3-chloro-N-(2,4-difluoro-3-(7- fluoro-3-(1H-imidazol-2-yl)- 1H-indazol-6-yl)phenyl)-1- methyl-1H-pyrazole-4- sulfonamide | 508 (M + H)⁺ |
| 2I | 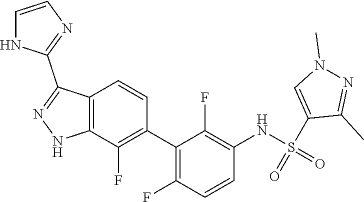 [SMILES: Cc1nn(C)cc1S(=O)(=O)Nc1ccc(F)c(-c2ccc3c(-c4ncc[nH]4)n[nH]c3c2F)c1F] | N-(2,4-difluoro-3-(7-fluoro-3- (1H-imidazol-2-yl)-1H- indazol-6-yl)phenyl)-1,3- dimethyl-1H-pyrazole-4- sulfonamide | 488 (M + H)⁺ |
| 2J | 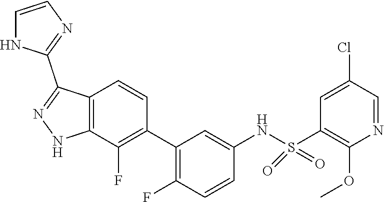 [SMILES: COc1ncc(Cl)cc1S(=O)(=O)Nc1ccc(F)c(-c2ccc3c(-c4ncc[nH]4)n[nH]c3c2F)c1] | 5-chloro-N-(4-fluoro-3-(7- fluoro-3-(1H-imidazol-2-yl)- 1H-indazol-6-yl)phenyl)-2- methoxy-pyridine-3- sulfonamide | 517 (M + H)⁺ |
| 2K | 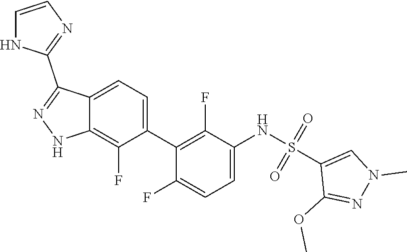 [SMILES: COc1nn(C)cc1S(=O)(=O)Nc1ccc(F)c(-c2ccc3c(-c4ncc[nH]4)n[nH]c3c2F)c1F] | N-(2,4-difluoro-3-(7-fluoro-3- (1H-imidazol-2-yl)-1H- indazol-6-yl)phenyl)-3- methoxy-1-methyl-1H- pyrazole-4-sulfonamide | 504 (M + H)⁺ |
| 2L | 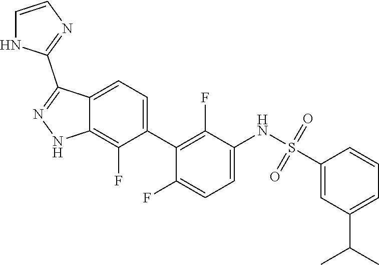 [SMILES: CC(C)c1cccc(S(=O)(=O)Nc2ccc(F)c(-c3ccc4c(-c5ncc[nH]5)n[nH]c4c3F)c2F)c1] | N-(2,4-difluoro-3-(7-fluoro-3- (1H-imidazol-2-yl)-1H- indazol-6-yl)phenyl)-3- isopropyl- benzenesulfonamide | 512 (M + H)⁺ |
| 2M | 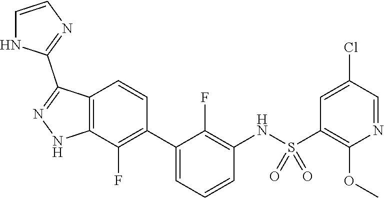 [SMILES: COc1ncc(Cl)cc1S(=O)(=O)Nc1cccc(-c2ccc3c(-c4ncc[nH]4)n[nH]c3c2F)c1F] | 5-chloro-N-(2-fluoro-3-(7- fluoro-3-(1H-imidazol-2-yl)- 1H-indazol-6-yl)phenyl)-2- methoxy-pyridine-3- sulfonamide | 517 (M + H)⁺ |
| 2N | 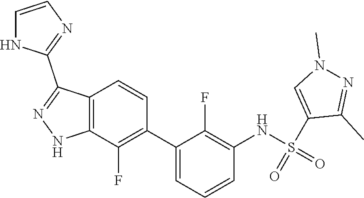 [SMILES: Cc1nn(C)cc1S(=O)(=O)Nc1cccc(-c2ccc3c(-c4ncc[nH]4)n[nH]c3c2F)c1F] | N-(2-fluoro-3-(7-fluoro-3- (1H-imidazol-2-yl)-1H- indazol-6-yl)phenyl)-1,3- dimethyl-1H-pyrazole-4- sulfonamide | 470 (M + H)⁺ |
| 2O | 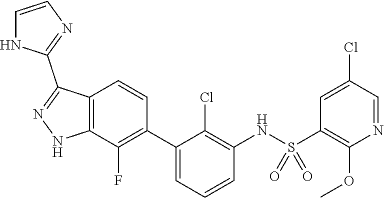 [SMILES: COc1ncc(Cl)cc1S(=O)(=O)Nc1cccc(-c2ccc3c(-c4ncc[nH]4)n[nH]c3c2F)c1Cl] | 5-chloro-N-(2-chloro-3-(7- fluoro-3-(1H-imidazol-2-yl)- 1H-indazol-6-yl)phenyl)-2- methoxy-pyridine-3- sulfonamide | 533 (M + H)⁺ |
| 2P | 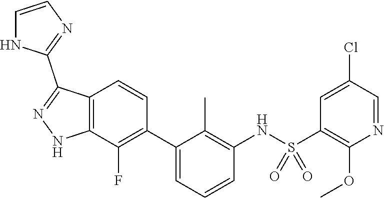 [SMILES: COc1ncc(Cl)cc1S(=O)(=O)Nc1cccc(-c2ccc3c(-c4ncc[nH]4)n[nH]c3c2F)c1C] | 5-chloro-N-(3-(7-fluoro-3- (1H-imidazol-2-yl)-1H- indazol-6-yl)-2- methylphenyl)-2- methoxypyridine-3- sulfonamide | 513 (M + H)⁺ |
| 2Q | 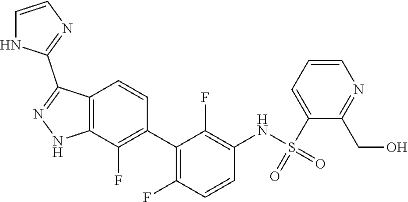 [SMILES: O=S(=O)(Nc1ccc(F)c(-c2ccc3c(-c4ncc[nH]4)n[nH]c3c2F)c1F)c1cccnc1CO] | N-(2,4-difluoro-3-(7-fluoro-3- (1H-imidazol-2-yl)-1H- indazol-6-yl)phenyl)-2- (hydroxyl-methyl)pyridine-3- sulfonamide | 501 (M + H)⁺ |
| 2R | 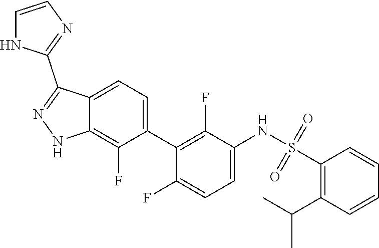 [SMILES: CC(C)c1ccccc1S(=O)(=O)Nc1ccc(F)c(-c2ccc3c(-c4ncc[nH]4)n[nH]c3c2F)c1F] | N-(2,4-difluoro-3-(7-fluoro-3- (1H-imidazol-2-yl)-1H- indazol-6-yl)phenyl)-3- isopropyl- benzenesulfonamide | 512 (M + H)⁺ |
| 2S | 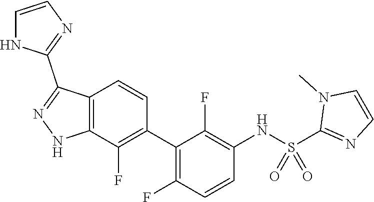 [SMILES: Cn1ccnc1S(=O)(=O)Nc1ccc(F)c(-c2ccc3c(-c4ncc[nH]4)n[nH]c3c2F)c1F] | N-(2,4-difluoro-3-(7-fluoro-3- (1H-imidazol-2-yl)-1H- indazol-6-yl)phenyl)-1- methyl-1H-imidazole-2- sulfonamide | 474 (M + H)⁺ |
| 2T | 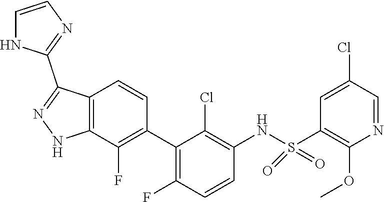 [SMILES: COc1ncc(Cl)cc1S(=O)(=O)Nc1ccc(F)c(-c2ccc3c(-c4ncc[nH]4)n[nH]c3c2F)c1Cl] | 5-chloro-N-(2-chloro-4- fluoro-3-(7-fluoro-3-(1H- imidazol-2-yl)-1H-indazol-6- yl)phenyl)-2- methoxypyridine-3- sulfonamide | 551 (M + H)⁺ |
| 2U | 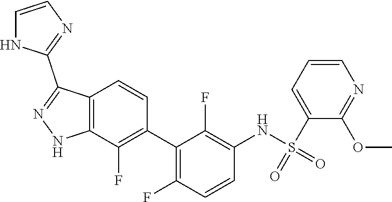 [SMILES: COc1ncccc1S(=O)(=O)Nc1ccc(F)c(-c2ccc3c(-c4ncc[nH]4)n[nH]c3c2F)c1F] | N-(2,4-difluoro-3-(7-fluoro-3- (1H-imidazol-2-yl)-1H- indazol-6-yl)phenyl)-2- methoxy-pyridine-3- sulfonamide | 501 (M + H)⁺ |
| 2V | 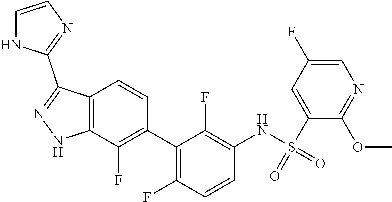 [SMILES: COc1ncc(F)cc1S(=O)(=O)Nc1ccc(F)c(-c2ccc3c(-c4ncc[nH]4)n[nH]c3c2F)c1F] | 5-fluoro-N-(2,4-difluoro-3-(7- fluoro-3-(1H-imidazol-2-yl)- 1H-indazol-6-yl)phenyl)-2- methoxy-pyridine-3- sulfonamide | 519 (M + H)⁺ |
| 2W | 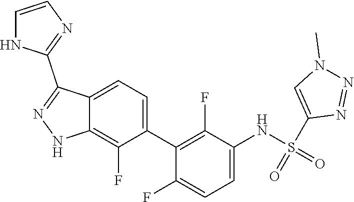 [SMILES: Cn1cc(S(=O)(=O)Nc2ccc(F)c(-c3ccc4c(-c5ncc[nH]5)n[nH]c4c3F)c2F)nn1] | N-(2,4-difluoro-3-(7-fluoro-3- (1H-imidazol-2-yl)-1H- indazol-6-yl)phenyl)-1- methyl-1H-1,2,3-triazole-4- sulfonamide | 475 (M + H)⁺ |
| 2X | 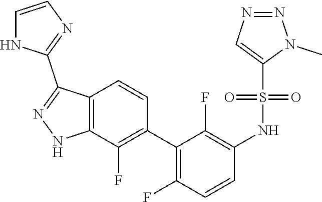 [SMILES: Cn1nncc1S(=O)(=O)Nc1ccc(F)c(-c2ccc3c(-c4ncc[nH]4)n[nH]c3c2F)c1F] | N-(2,4-difluoro-3-(7-fluoro-3- (1H-imidazol-2-yl)-1H- indazol-6-yl)phenyl)-1- methyl-1H-1,2,3-triazole-5- sulfonamide | 475 (M + H)⁺ |
| 2Y | 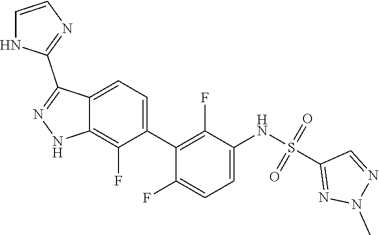 [SMILES: Cn1ncc(S(=O)(=O)Nc2ccc(F)c(-c3ccc4c(-c5ncc[nH]5)n[nH]c4c3F)c2F)n1] | N-(2,4-difluoro-3-(7-fluoro-3- (1H-imidazol-2-yl)-1H- indazol-6-yl)phenyl)-2- methyl-2H-l,2,3-triazole-4- sulfonamide | 475 (M + H)⁺ |
| 2Z | 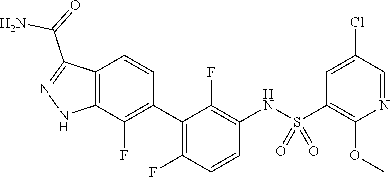 [SMILES: COc1ncc(Cl)cc1S(=O)(=O)Nc1ccc(F)c(-c2ccc3c(C(N)=O)n[nH]c3c2F)c1F] | 6-(3-((5-chloro-2-methoxy- pyridine)-3-sulfonamido)-2,6- difluorophenyl)-7-fluoro-1H- indazole-3-carboxamide | 512 (M + H)⁺ |
| 2AA | 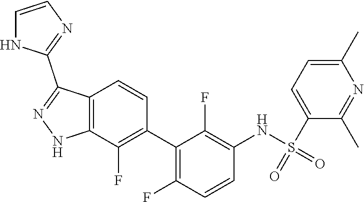 [SMILES: Cc1ccc(S(=O)(=O)Nc2ccc(F)c(-c3ccc4c(-c5ncc[nH]5)n[nH]c4c3F)c2F)c(C)n1] | N-(2,4-difluoro-3-(7-fluoro-3- (1H-imidazol-2-yl)-1H- indazol-6-yl)phenyl)-2,6- dimethyl-pyridine-3- sulfonamide | 499 (M + H)⁺ |
| 2AB | 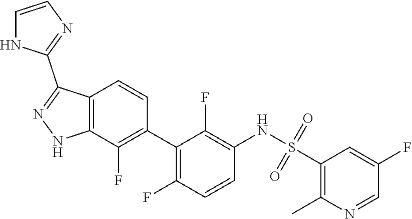 [SMILES: Cc1ncc(F)cc1S(=O)(=O)Nc1ccc(F)c(-c2ccc3c(-c4ncc[nH]4)n[nH]c3c2F)c1F] | N-(2,4-difluoro-3-(7-fluoro-3- (1H-imidazol-2-yl)-1H- indazol-6-yl)phenyl)-5-fluoro- 2-methylpyridine-3- sulfonamide | 503 (M + H)⁺ |
| 2AC | 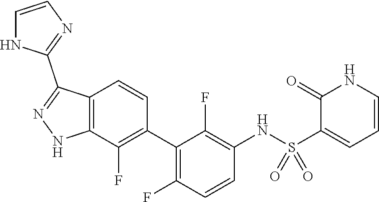 [SMILES: O=c1[nH]cccc1S(=O)(=O)Nc1ccc(F)c(-c2ccc3c(-c4ncc[nH]4)n[nH]c3c2F)c1F] | N-(2,4-difluoro-3-(7-fluoro-3- (1H-imidazol-2-yl)-1H- indazol-6-yl)phenyl)-2-oxo- 1,2-dihydro-pyridine-3- sulfonamide | 487 (M + H)⁺ |
| 2AD | 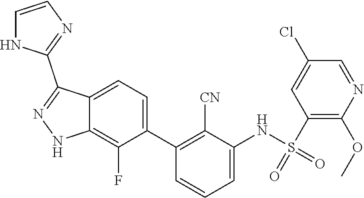 [SMILES: COc1ncc(Cl)cc1S(=O)(=O)Nc1cccc(-c2ccc3c(-c4ncc[nH]4)n[nH]c3c2F)c1C#N] | 5-chloro-N-(2-cyano-3-(7- fluoro-3-(1H-imidazol-2-yl)- 1H-indazol-6-yl)phenyl)-2- methoxypyridine-3- sulfonamide | 524 (M + H)⁺ |
| 2AE | 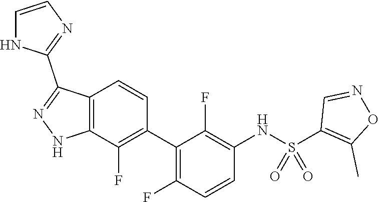 [SMILES: Cc1oncc1S(=O)(=O)Nc1ccc(F)c(-c2ccc3c(-c4ncc[nH]4)n[nH]c3c2F)c1F] | N-(2,4-difluoro-3-(7-fluoro-3- (1H-imidazol-2-yl)-1H- indazol-6-yl)phenyl)-5- methylisoxazole-4- sulfonamide | 475 (M + H)⁺ |
| 2AF | 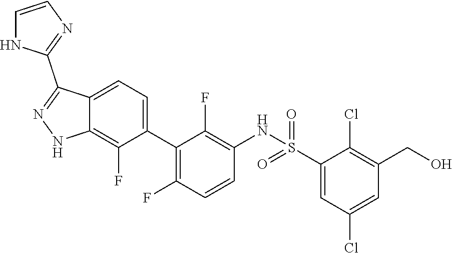 [SMILES: O=S(=O)(Nc1ccc(F)c(-c2ccc3c(-c4ncc[nH]4)n[nH]c3c2F)c1F)c1cc(Cl)cc(CO)c1Cl] | 2,5-dichloro-N-(2,4-difluoro- 3-(7-fluoro-3-(1H-imidazol-2- yl)-1H-indazol-6-yl)-phenyl)- 3-(hydroxyl- methyl)benzenesulfonamide | 568 (M + H)⁺ |
| 2AG | 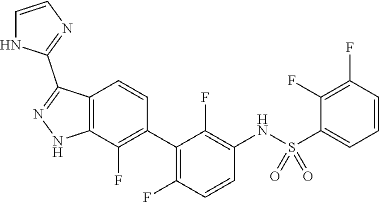 [SMILES: O=S(=O)(Nc1ccc(F)c(-c2ccc3c(-c4ncc[nH]4)n[nH]c3c2F)c1F)c1cccc(F)c1F] | N-(2,4-difluoro-3-(7-fluoro-3- (1H-imidazol-2-yl)-1H- indazol-6-yl)phenyl)-2,3- difluorobenzenesulfonamide | 506 (M + H)⁺ |
| 2AH | 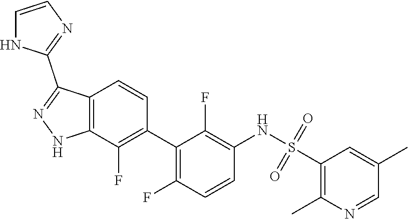 [SMILES: Cc1cnc(C)c(S(=O)(=O)Nc2ccc(F)c(-c3ccc4c(-c5ncc[nH]5)n[nH]c4c3F)c2F)c1] | N-(2,4-difluoro-3-(7-fluoro-3- (1H-imidazol-2-yl)-1H- indazol-6-yl)phenyl)-2,5- dimethyl-pyridine-3- sulfonamide | 499 (M + H)⁺ |
| 2AI | 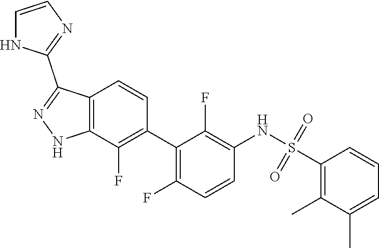 [SMILES: Cc1cccc(S(=O)(=O)Nc2ccc(F)c(-c3ccc4c(-c5ncc[nH]5)n[nH]c4c3F)c2F)c1C] | N-(2,4-difluoro-3-(7-fluoro-3- (1H-imidazol-2-yl)-1H- indazol-6-yl)phenyl)-2,3- dimethylbenzenesulfonamide | 498 (M + H)⁺ |
| 2AJ | 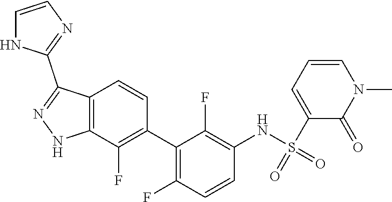 [SMILES: Cn1cccc(S(=O)(=O)Nc2ccc(F)c(-c3ccc4c(-c5ncc[nH]5)n[nH]c4c3F)c2F)c1=O] | N-(2,4-difluoro-3-(7-fluoro-3- (1H-imidazol-2-yl)-1H- indazol-6-yl)phenyl)-1- methyl-2-oxo-1,2-dihydro- pyridine-3-sulfonamide | 501 (M + H)⁺ |
| 2AK | 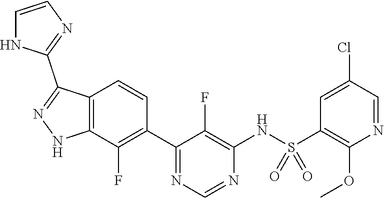 [SMILES: COc1ncc(Cl)cc1S(=O)(=O)Nc1ncnc(-c2ccc3c(-c4ncc[nH]4)n[nH]c3c2F)c1F] | 5-chloro-N-(5-fluoro-6-(7- fluoro-3-(1H-imidazol-2-yl)- 1H-indazol-6-yl)pyrimidin-4- yl)-2-methoxypyridine-3- sulfonamide | 519 (M + H)⁺ |
| 2AL | 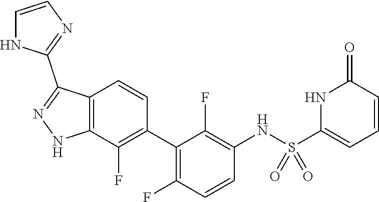 [SMILES: O=c1cccc(S(=O)(=O)Nc2ccc(F)c(-c3ccc4c(-c5ncc[nH]5)n[nH]c4c3F)c2F)[nH]1] | N-(2,4-difluoro-3-(7-fluoro-3- (1H-imidazol-2-yl)-1H- indazol-6-yl)phenyl)-6-oxo- 1,6-dihydropyridine-2- sulfonamide | 487 (M + H)⁺ |
| 2AM | 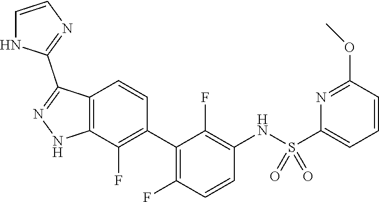 [SMILES: COc1cccc(S(=O)(=O)Nc2ccc(F)c(-c3ccc4c(-c5ncc[nH]5)n[nH]c4c3F)c2F)n1] | N-(2,4-difluoro-3-(7-fluoro-3- (1H-imidazol-2-yl)-1H- indazol-6-yl)phenyl)-6- methoxypyridine-2- sulfonamide | 501 (M + H)⁺ |
| 2AN | 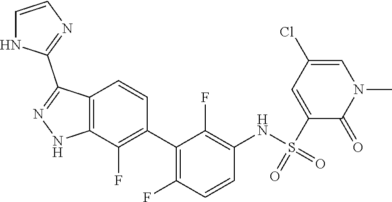 [SMILES: Cn1cc(Cl)cc(S(=O)(=O)Nc2ccc(F)c(-c3ccc4c(-c5ncc[nH]5)n[nH]c4c3F)c2F)c1=O] | 5-chloro-N-(2,4-difluoro-3-(7- fluoro-3-(1H-imidazol-2-yl)- 1H-indazol-6-yl)phenyl)-1- methyl-2-oxo-1,2-dihydro- pyridine-3-sulfonamide | 535 (M + H)⁺ |
| 2AO | 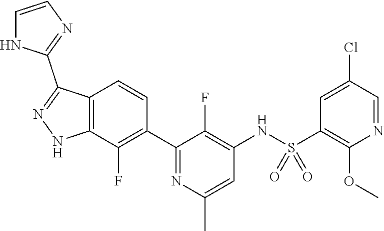 [SMILES: COc1ncc(Cl)cc1S(=O)(=O)Nc1cc(C)nc(-c2ccc3c(-c4ncc[nH]4)n[nH]c3c2F)c1F] | 5-chloro-N-(3-fluoro-2-(7- fluoro-3-(1H-imidazol-2-yl)- 1H-indazol-6-yl)-6-methyl- pyridin-4-yl)-2-methoxy- pyridine-3-sulfonamide | 532 (M + H)⁺ |
| 2AP | 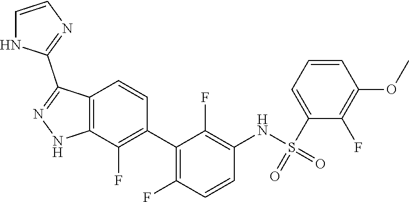 [SMILES: COc1cccc(S(=O)(=O)Nc2ccc(F)c(-c3ccc4c(-c5ncc[nH]5)n[nH]c4c3F)c2F)c1F] | N-(2,4-difluoro-3-(7-fluoro-3- (1H-imidazol-2-yl)-1H- indazol-6-yl)phenyl)-2-fluoro- 3-methoxybenzene- sulfonamide | 518 (M + H)⁺ |
| 2AQ | 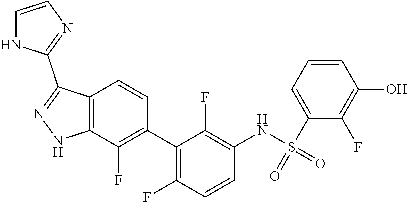 [SMILES: O=S(=O)(Nc1ccc(F)c(-c2ccc3c(-c4ncc[nH]4)n[nH]c3c2F)c1F)c1cccc(O)c1F] | N-(2,4-difluoro-3-(7-fluoro-3- (1H-imidazol-2-yl)-1H- indazol-6-yl)phenyl)-2-fluoro- 3-hydroxybenzene- sulfonamide | 504 (M + H)⁺ |
| 2AR | 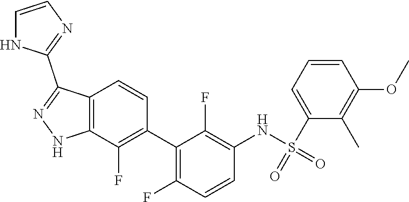 [SMILES: COc1cccc(S(=O)(=O)Nc2ccc(F)c(-c3ccc4c(-c5ncc[nH]5)n[nH]c4c3F)c2F)c1C] | N-(2,4-difluoro-3-(7-fluoro-3- (1H-imidazol-2-yl)-1H- indazol-6-yl)phenyl)-3- methoxy-2-methylbenzene- sulfonamide | 514 (M + H)⁺ |
| 2AS | 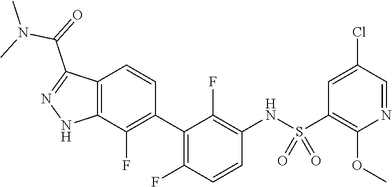 | 6-(3-((5-chloro-2-methoxy- pyridine)-3-sulfonamido)-2,6- difluorophenyl)-7-fluoro-N,N- dimethyl-1H-indazole-3- carboxamide | 540 (M + H)⁺ |
| 2AT | 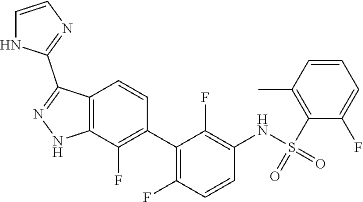 [SMILES: Cc1cccc(F)c1S(=O)(=O)Nc1ccc(F)c(-c2ccc3c(-c4ncc[nH]4)n[nH]c3c2F)c1F] | N-(2,4-difluoro-3-(7-fluoro-3- (1H-imidazol-2-yl)-1H- indazol-6-yl)phenyl)-2-fluoro- 6-methylbenzene-sulfonamide | 502 (M + H)⁺ |
| 2AU | 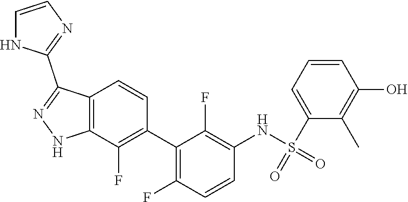 [SMILES: Cc1c(O)cccc1S(=O)(=O)Nc1ccc(F)c(-c2ccc3c(-c4ncc[nH]4)n[nH]c3c2F)c1F] | N-(2,4-difluoro-3-(7-fluoro-3- (1H-imidazol-2-yl)-1H- indazol-6-yl)phenyl)-3- hydroxy-2-methylbenzene- sulfonamide | 500 (M + H)⁺ |
| 2AV | 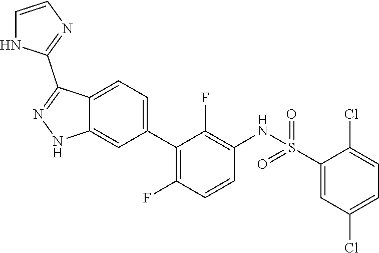 [SMILES: O=S(=O)(Nc1ccc(F)c(-c2ccc3c(-c4ncc[nH]4)n[nH]c3c2)c1F)c1cc(Cl)ccc1Cl] | N-(3-(3-(1H-imidazol-2-yl)- 1H-indazol-6-yl)-2,4- difluorophenyl)-2,5- dichlorobenzenesulfonamide | 520 (M + H)⁺ |
| 2AW | 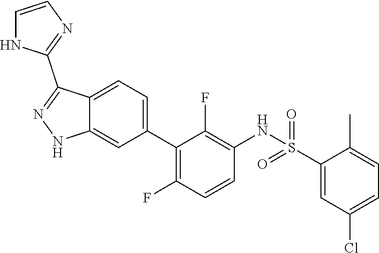 [SMILES: Cc1ccc(Cl)cc1S(=O)(=O)Nc1ccc(F)c(-c2ccc3c(-c4ncc[nH]4)n[nH]c3c2)c1F] | N-(3-(3-(1H-imidazol-2-yl)- 1H-indazol-6-yl)-2,4- difluorophenyl)-5-chloro-2- methylbenzenesulfonamide | 500 (M + H)⁺ |
| 2AX | 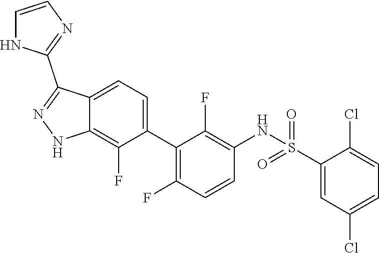 [SMILES: O=S(=O)(Nc1ccc(F)c(-c2ccc3c(-c4ncc[nH]4)n[nH]c3c2F)c1F)c1cc(Cl)ccc1Cl] | 2,5-dichloro-N-(2,4-difluoro- 3-(7-fluoro-3-(1H-imidazol-2- yl)-1H-indazol-6-yl)phenyl)- benzenesulfonamide | 538 (M + H)⁺ |
| 2AY | 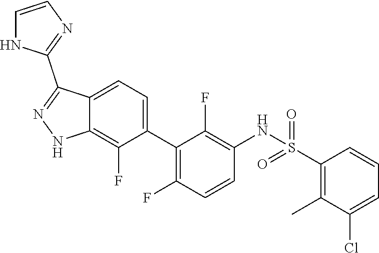 [SMILES: Cc1c(Cl)cccc1S(=O)(=O)Nc1ccc(F)c(-c2ccc3c(-c4ncc[nH]4)n[nH]c3c2F)c1F] | 3-chloro-N-(2,4-difluoro-3-(7- fluoro-3-(1H-imidazol-2-yl)- 1H-indazol-6-yl)phenyl)-2- methylbenzenesulfonamide | 518 (M + H)⁺ |
| 2AZ | 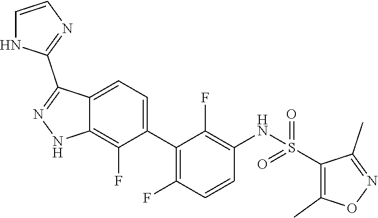 [SMILES: Cc1noc(C)c1S(=O)(=O)Nc1ccc(F)c(-c2ccc3c(-c4ncc[nH]4)n[nH]c3c2F)c1F] | N-(2,4-difluoro-3-(7-fluoro-3- (1H-imidazol-2-yl)-1H- indazol-6-yl)phenyl)-3,5- dimethylisoxazole-4- sulfonamide | 489 (M + H)⁺ |
| 2BA | 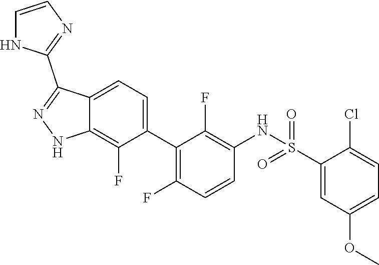 [SMILES: COc1ccc(Cl)c(S(=O)(=O)Nc2ccc(F)c(-c3ccc4c(-c5ncc[nH]5)n[nH]c4c3F)c2F)c1] | 2-chloro-N-(2,4-difluoro-3-(7- fluoro-3-(1H-imidazol-2-yl)- 1H-indazol-6-yl)phenyl)-5- methoxybenzene-sulfonamide | 534 (M + H)⁺ |
| 2BB | 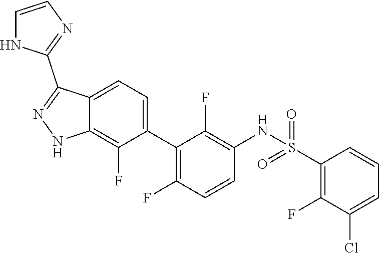 [SMILES: O=S(=O)(Nc1ccc(F)c(-c2ccc3c(-c4ncc[nH]4)n[nH]c3c2F)c1F)c1cccc(Cl)c1F] | 3-chloro-N-(2,4-difluoro-3-(7- fluoro-3-(1H-imidazol-2-yl)- 1H-indazol-6-yl)phenyl)-2- fluorobenzenesulfonamide | 522 (M + H)⁺ |
| 2BC | 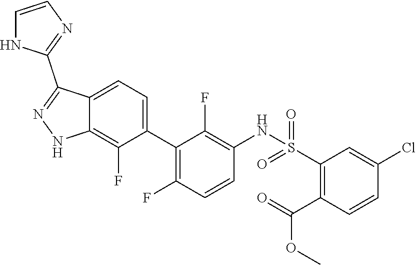 [SMILES: COC(=O)c1ccc(Cl)cc1S(=O)(=O)Nc1ccc(F)c(-c2ccc3c(-c4ncc[nH]4)n[nH]c3c2F)c1F] | methyl 4-chloro-2-(N-(2,4- difluoro-3-(7-fluoro-3-(1H- imidazol-2-yl)-1H-indazol-6- yl)phenyl)sulfamoyl)benzoate | 562 (M + H)⁺ |
| 2BD | 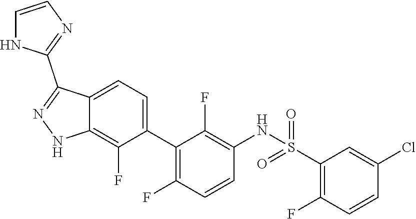 [SMILES: O=S(=O)(Nc1ccc(F)c(-c2ccc3c(-c4ncc[nH]4)n[nH]c3c2F)c1F)c1cc(Cl)ccc1F] | 5-chloro-N-(2,4-difluoro-3-(7- fluoro-3-(1H-imidazol-2-yl)- 1H-indazol-6-yl)phenyl)-2- fluorobenzenesulfonamide | 522 (M + H)⁺ |
| 2BE | 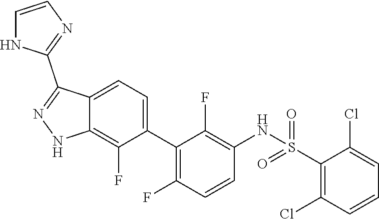 [SMILES: O=S(=O)(Nc1ccc(F)c(-c2ccc3c(-c4ncc[nH]4)n[nH]c3c2F)c1F)c1c(Cl)cccc1Cl] | 2,5-dichloro-N-(2,4-difluoro- 3-(7-fluoro-3-(1H-imidazol-2- yl)-1H-indazol-6-yl)phenyl)- benzenesulfonamide | 538 (M + H)⁺ |
| 2BF | 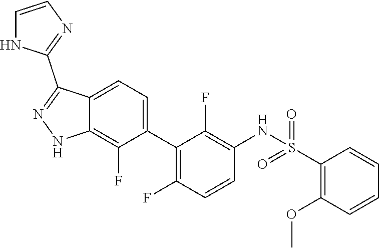 [SMILES: COc1ccccc1S(=O)(=O)Nc1ccc(F)c(-c2ccc3c(-c4ncc[nH]4)n[nH]c3c2F)c1F] | N-(2,4-difluoro-3-(7-fIuoro-3- (1H-imidazol-2-yl)-1H- indazol-6-yl)phenyl)-2- methoxybenzenesulfonamide | 500 (M + H)⁺ |
| 2BG | 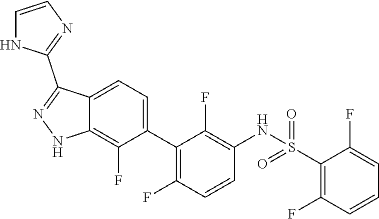 [SMILES: O=S(=O)(Nc1ccc(F)c(-c2ccc3c(-c4ncc[nH]4)n[nH]c3c2F)c1F)c1c(F)cccc1F] | N-(2,4-difluoro-3-(7-fluoro-3- (1H-imidazol-2-yl)-1H- indazol-6-yl)phenyl)-2,6- difluorobenzenesulfonamide | 506 (M + H)⁺ |
| 2BH | 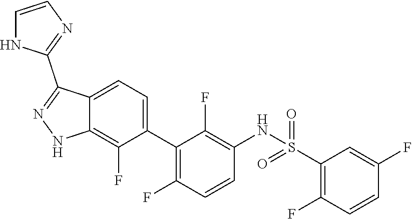 [SMILES: O=S(=O)(Nc1ccc(F)c(-c2ccc3c(-c4ncc[nH]4)n[nH]c3c2F)c1F)c1cc(F)ccc1F] | N-(2,4-difluoro-3-(7-fluoro-3- (1H-imidazol-2-yl)-1H- indazol-6-yl)phenyl)-2,5- difluorobenzenesulfonamide | 506 (M + H)⁺ |
| 2BI | 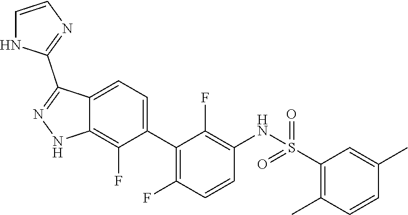 [SMILES: Cc1ccc(C)c(S(=O)(=O)Nc2ccc(F)c(-c3ccc4c(-c5ncc[nH]5)n[nH]c4c3F)c2F)c1] | N-(2,4-difluoro-3-(7-fluoro-3- (1H-imidazol-2-yl)-1H- indazol-6-yl)phenyl)-2,5- dimethylbenzenesulfonamide | 498 (M + H)⁺ |
| 2BJ | 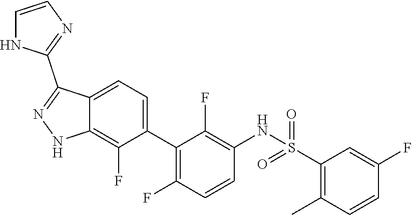 [SMILES: Cc1ccc(F)cc1S(=O)(=O)Nc1ccc(F)c(-c2ccc3c(-c4ncc[nH]4)n[nH]c3c2F)c1F] | N-(2,4-difluoro-3-(7-fluoro-3- (1H-imidazol-2-yl)-1H- indazol-6-yl)phenyl)-5-fluoro- 2-methylbenzenesulfonamide | 502 (M + H)⁺ |
| 2BK | 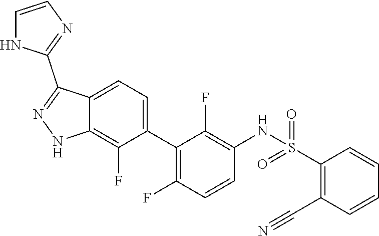 [SMILES: N#Cc1ccccc1S(=O)(=O)Nc1ccc(F)c(-c2ccc3c(-c4ncc[nH]4)n[nH]c3c2F)c1F] | 2-cyano-N-(2,4-difluoro-3-(7- fluoro-3-(1H-imidazol-2-yl)- 1H-indazol-6-yl)phenyl)- benzenesulfonamide | 495 (M + H)⁺ |
| 2BL | 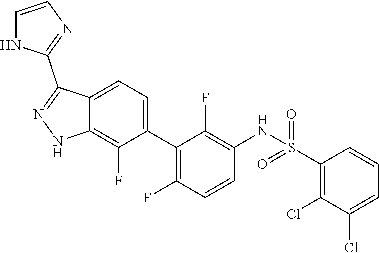 [SMILES: O=S(=O)(Nc1ccc(F)c(-c2ccc3c(-c4ncc[nH]4)n[nH]c3c2F)c1F)c1cccc(Cl)c1Cl] | 2,3-dichloro-N-(2,4-difluoro- 3-(7-fluoro-3-(1H-imidazol-2- yl)-1H-indazol-6-yl)phenyl)- benzenesulfonamide | 538 (M + H)⁺ |
| 2BM | 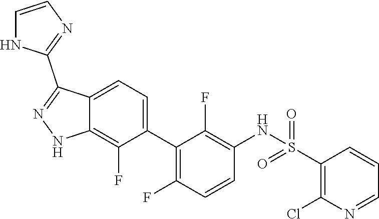 [SMILES: O=S(=O)(Nc1ccc(F)c(-c2ccc3c(-c4ncc[nH]4)n[nH]c3c2F)c1F)c1cccnc1Cl] | 2-chloro-N-(2,4-difluoro-3-(7- fluoro-3-(1H-imidazol-2-yl)- 1H-indazol-6-yl)phenyl)- pyridine-3-sulfonamide | 505 (M + H)⁺ |
| 2BN | 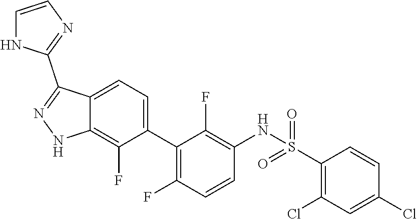 [SMILES: O=S(=O)(Nc1ccc(F)c(-c2ccc3c(-c4ncc[nH]4)n[nH]c3c2F)c1F)c1ccc(Cl)cc1Cl] | 2,4-dichloro-N-(2,4-difluoro- 3-(7-fluoro-3-(1H-imidazol-2- yl)-1H-indazol-6-yl)phenyl)- benzenesulfonamide | 538 (M + H)⁺ |
| 2BO | 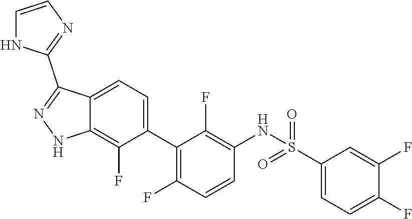 [SMILES: O=S(=O)(Nc1ccc(F)c(-c2ccc3c(-c4ncc[nH]4)n[nH]c3c2F)c1F)c1ccc(F)c(F)c1] | N-(2,4-difluoro-3-(7-fluoro-3- (1H-imidazol-2-yl)-1H- indazol-6-yl)phenyl)-3,4- difluorobenzenesulfonamide | 506 (M + H)⁺ |
| 2BP | 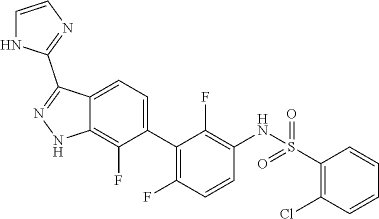 [SMILES: O=S(=O)(Nc1ccc(F)c(-c2ccc3c(-c4ncc[nH]4)n[nH]c3c2F)c1F)c1ccccc1Cl] | 2-chloro-N-(2,4-difluoro-3-(7- fluoro-3-(1H-imidazol-2-yl)- 1H-indazol-6-yl)phenyl)- benzenesulfonamide | 504 (M + H)⁺ |
| 2BQ | 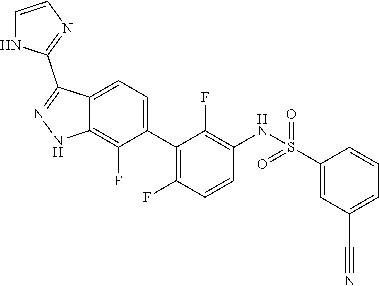 [SMILES: N#Cc1cccc(S(=O)(=O)Nc2ccc(F)c(-c3ccc4c(-c5ncc[nH]5)n[nH]c4c3F)c2F)c1] | 3-cyano-N-(2,4-difluoro-3-(7- fluoro-3-(1H-imidazol-2-yl)- 1H-indazol-6-yl)phenyl)- benzenesulfonamide | 495 (M + H)⁺ |
| 2BR | 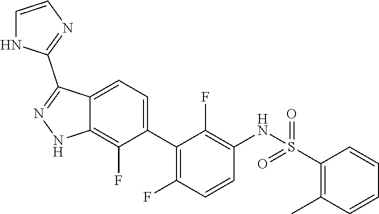 [SMILES: Cc1ccccc1S(=O)(=O)Nc1ccc(F)c(-c2ccc3c(-c4ncc[nH]4)n[nH]c3c2F)c1F] | N-(2,4-difluoro-3-(7-fluoro-3- (1H-imidazol-2-yl)-1H- indazol-6-yl)phenyl)-2- methylbenzenesulfonamide | 484 (M + H)⁺ |
| 2BS | 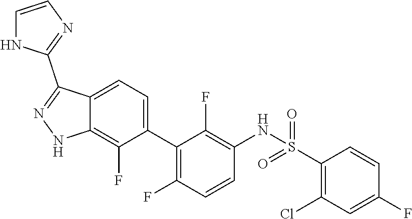 [SMILES: O=S(=O)(Nc1ccc(F)c(-c2ccc3c(-c4ncc[nH]4)n[nH]c3c2F)c1F)c1ccc(F)cc1Cl] | 2-chloro-N-(2,4-difluoro-3-(7- fluoro-3-1H-imidazol-2-yl)- 1H-indazol-6-yl)phenyl)-4- fluorobenzenesulfonamide | 522 (M + H)⁺ |
| 2BT | 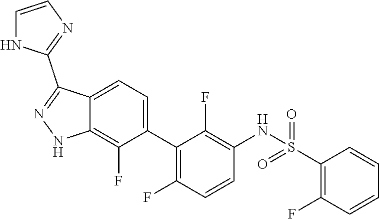 [SMILES: O=S(=O)(Nc1ccc(F)c(-c2ccc3c(-c4ncc[nH]4)n[nH]c3c2F)c1F)c1ccccc1F] | N-(2,4-difluoro-3-(7-fluoro-3- (1H-imidazol-2-yl)-1H- indazol-6-yl)phenyl)-2- fluorobenzenesulfonamide | 488 (M + H)⁺ |
| 2BU | 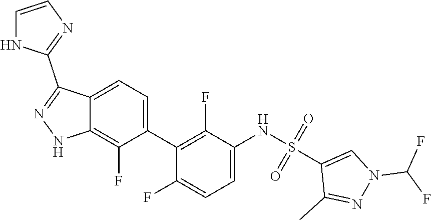 [SMILES: Cc1nn(C(F)F)cc1S(=O)(=O)Nc1ccc(F)c(-c2ccc3c(-c4ncc[nH]4)n[nH]c3c2F)c1F] | N-(2,4-difluoro-3-(7-fluoro-3- (1H-imidazol-2-yl)-1H- indazol-6-yl)phenyl)-1- (difluoromethyl)-3-methyl- 1H-pyrazole-4-sulfonamide | 524 (M + H)⁺ |
| 2BV | 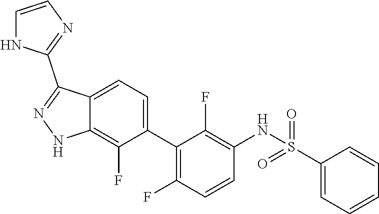 [SMILES: O=S(=O)(Nc1ccc(F)c(-c2ccc3c(-c4ncc[nH]4)n[nH]c3c2F)c1F)c1ccccc1] | N-(2,4-difluoro-3-(7-fluoro-3- (1H-imidazol-2-yl)-1H- indazol-6-yl)phenyl)benzene- sulfonamide | 470 (M + H)⁺ |
| 2BW | 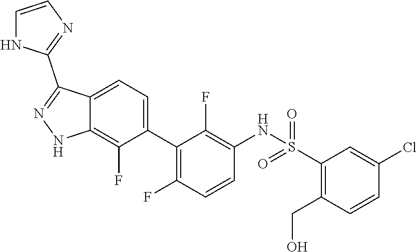 [SMILES: O=S(=O)(Nc1ccc(F)c(-c2ccc3c(-c4ncc[nH]4)n[nH]c3c2F)c1F)c1cc(Cl)ccc1CO] | 5-chloro-N-(2,4-difluoro-3-(7- fluoro-3-(1H-imidazol-2-yl)- 1H-indazol-6-yl)phenyl)-2- (hydroxyl-methyl)benzene- sulfonamide | 534 (M + H)⁺ |
| 2BX | 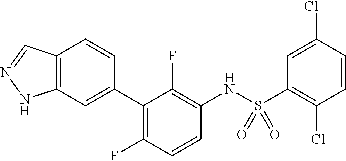 [SMILES: O=S(=O)(Nc1ccc(F)c(-c2ccc3cn[nH]c3c2)c1F)c1cc(Cl)ccc1Cl] | 2,5-dichloro-N-(2,4-difluoro- 3-(1H-indazol-6- yl)phenyl)benzenesulfonamide | 455 (M + H)⁺ |
| 2BY | 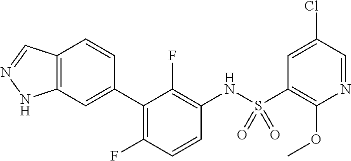 [SMILES: COc1ncc(Cl)cc1S(=O)(=O)Nc1ccc(F)c(-c2ccc3cn[nH]c3c2)c1F] | 5-chloro-N-(2,4-difluoro-3- (1H-indazol-6-yl)-phenyl)-2- methoxypyridine-3- sulfonamide | 451 (M + H)⁺ |
| 2BZ | 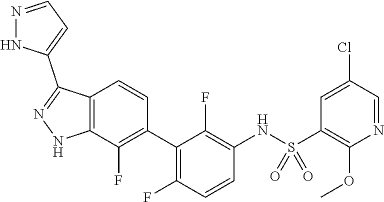 [SMILES: COc1ncc(Cl)cc1S(=O)(=O)Nc1ccc(F)c(-c2ccc3c(-c4ccn[nH]4)n[nH]c3c2F)c1F] | 5-chloro-N-(2,4-difluoro-3-(7- fluoro-3-(1H-pyrazol-5-yl)- 1H-indazol-6-yl)phenyl)-2- methoxypyridine-3- sulfonamide | 517 (M + H)⁺ |
| 2CA | 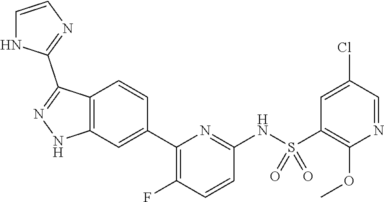 [SMILES: COc1ncc(Cl)cc1S(=O)(=O)Nc1ccc(F)c(-c2ccc3c(-c4ncc[nH]4)n[nH]c3c2)n1] | N-(6-(3-1H-imidazol-2-yl)- 1H-indazol-6-yl)-5-fluoro- pyridin-2-yl)-5-chloro-2- methoxypyridine-3- sulfonamide | 500 (M + H)⁺ |
| 2CB | 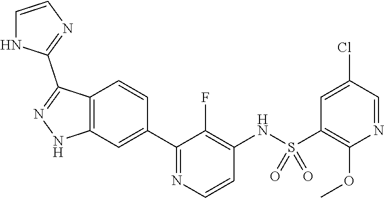 [SMILES: COc1ncc(Cl)cc1S(=O)(=O)Nc1ccnc(-c2ccc3c(-c4ncc[nH]4)n[nH]c3c2)c1F] | N-(2-(3-(1H-imidazol-2-yl)- 1H-indazol-6-yl)-3-fluoro- pyridin-4-yl)-5-chloro-2- methoxypyridinc-3- sulfonamide | 500 (M + H)⁺ |
| 2CC | 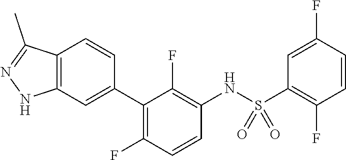 [SMILES: Cc1n[nH]c2cc(-c3c(F)ccc(NS(=O)(=O)c4cc(F)ccc4F)c3F)ccc12] | N-(2,4-difluoro-3-(3-methyl- 1H-indazol-6-yl)phenyl)-2,5- difluorobenzenesulfonamide | 436 (M + H)⁺ |
| 2CD | 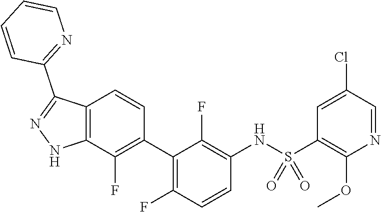 [SMILES: COc1ncc(Cl)cc1S(=O)(=O)Nc1ccc(F)c(-c2ccc3c(-c4ccccn4)n[nH]c3c2F)c1F] | 5-chloro-N-(2,4-difluoro-3-(7- fluoro-3-(pyridin-2-yl)-1H- indazol-6-yl)phenyl)-2- methoxypyridine-3- sulfonamide | 546 (M + H)⁺ |
| 2CE | 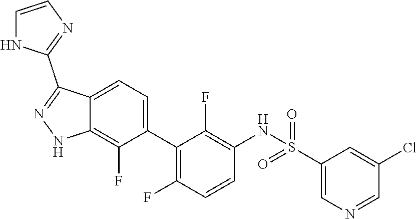 [SMILES: O=S(=O)(Nc1ccc(F)c(-c2ccc3c(-c4ncc[nH]4)n[nH]c3c2F)c1F)c1cncc(Cl)c1] | 5-chloro-N-(2,4-difluoro-3-(7- fluoro-3-1H-imidazol-2-yl)- 1H-indazol-6-yl)phenyl)- pyridine-3-sulfonamide | 505 (M + H)⁺ |
| 2CF | 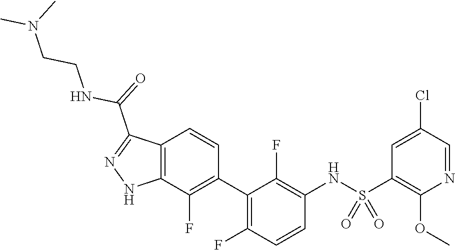 | 6-(3-((5-chloro-2-methoxy- pyridine)-3-sulfonamido)-2,6- difluorophenyl)-N-(2-(di- methylamino)ethyl)-7-fluoro- 1H-indazole-3-carboxamide | 583 (M + H)⁺ |
| 2CG | 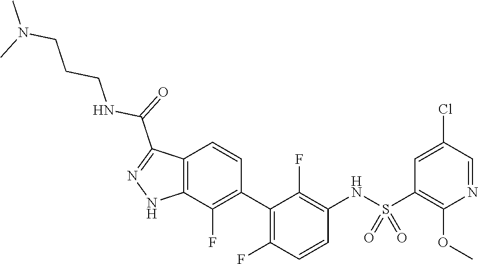 | 6-(3-((5-chloro-2-methoxy- pyridine)-3-sulfonamido)-2,6- difluorophenyl)-N-(3-(di- methylamino)propyl)-7- fluoro-1H-indazole-3- carboxamide | 597 (M + H)⁺ |
| 2CH | 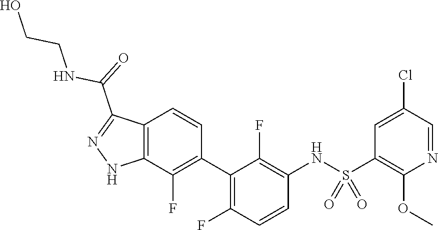 [SMILES: COc1ncc(Cl)cc1S(=O)(=O)Nc1ccc(F)c(-c2ccc3c(C(=O)NCCO)n[nH]c3c2F)c1F] | 6-(3-((5-chloro-2-methoxy- pyridine)-3-sulfonamido)-2,6- difluorophenyl)-7-fluoro-N- (2-hydroxyethyl)-1H- indazole-3-carboxamide | 556 (M + H)⁺ |
| 2CI | 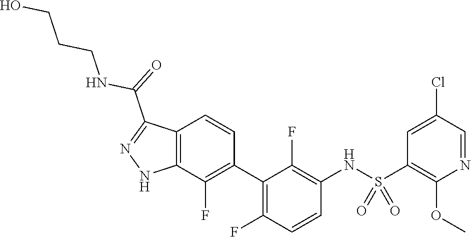 [SMILES: COc1ncc(Cl)cc1S(=O)(=O)Nc1ccc(F)c(-c2ccc3c(C(=O)NCCCO)n[nH]c3c2F)c1F] | 6-(3-((5-chloro-2-methoxy- pyridine)-3-sulfonamido)-2,6- difluorophenyl)-7-fluoro-N- (3-hydroxypropyl)-1H- indazole-3-carboxamide | 570 (M + H)⁺ |
| 2CJ | 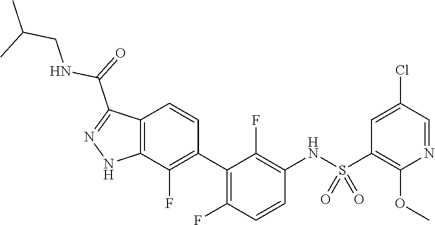 [SMILES: COc1ncc(Cl)cc1S(=O)(=O)Nc1ccc(F)c(-c2ccc3c(C(=O)NCC(C)C)n[nH]c3c2F)c1F] | 6-(3-((5-chloro-2-methoxy- pyridine)-3-sulfonamido)-2,6- difluorophenyl)-7-fluoro-N- isobutyl-1H-indazole-3- carboxamide | 568 (M + H)⁺ |
| 2CK | 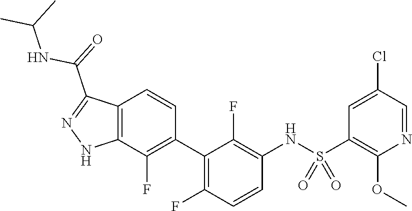 [SMILES: COc1ncc(Cl)cc1S(=O)(=O)Nc1ccc(F)c(-c2ccc3c(C(=O)NC(C)C)n[nH]c3c2F)c1F] | 6-(3-((5-chloro-2-methoxy- pyridine)-3-sulfonamido)-2,6- difluorophenyl)-7-fluoro-N- isopropyl-1H-indazole-3- carboxamide | 554 (M + H)⁺ |
| 2CL | 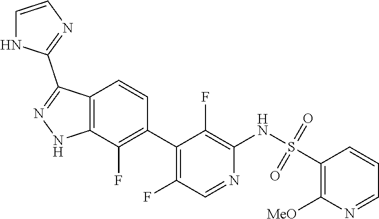 [SMILES: COc1ncccc1S(=O)(=O)Nc1ncc(F)c(-c2ccc3c(-c4ncc[nH]4)n[nH]c3c2F)c1F] | N-(3,5-difluoro-4-(7-fluoro-3- (1H-imidazol-2-yl)-1H- indazol-6-yl)pyridin-2-yl)-2- methoxypyridine-3- sulfonamide | 502 (M + H)⁺ |
| 2CM | 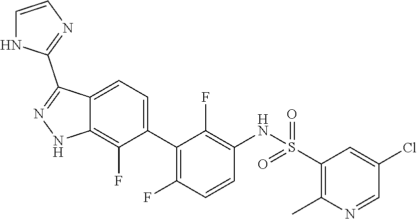 [SMILES: Cc1ncc(Cl)cc1S(=O)(=O)Nc1ccc(F)c(-c2ccc3c(-c4ncc[nH]4)n[nH]c3c2F)c1F] | 5-chloro-N-(2,4-difluoro-3-(7- fluoro-3-(1H-imidazol-2-yl)- 1H-indazol-6-yl)phenyl)-2- methylpyridine-3-sulfonamide | 519 (M + H)⁺ |
| 2CN | 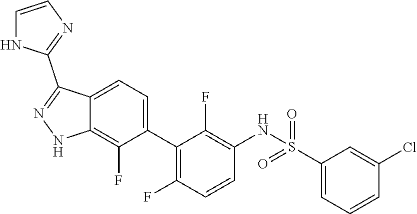 [SMILES: O=S(=O)(Nc1ccc(F)c(-c2ccc3c(-c4ncc[nH]4)n[nH]c3c2F)c1F)c1cccc(Cl)c1] | 3-chloro-N-(2,4-difluoro-3-(7- fluoro-3-(1H-imidazol-2-yl)- 1H-indazol-6-yl)phenyl)ben- zenesulfonamide | 504 (M + H)⁺ |
| 2CO | 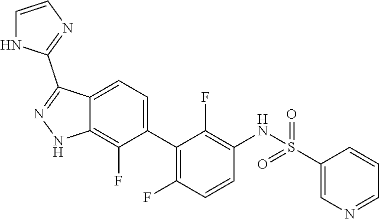 [SMILES: O=S(=O)(Nc1ccc(F)c(-c2ccc3c(-c4ncc[nH]4)n[nH]c3c2F)c1F)c1cccnc1] | N-(2,4-difluoro-3-(7-fluoro-3- (1H-imidazol-2-yl)-1H- indazol-6-yl)phenyl)pyridine- 3-sulfonamide | 471 (M + H)⁺ |

TABLE 4

| Compd | |
|---|---|
| No. | Physical Characterization Data |
| 2A | ¹H NMR (300 MHz, CD₃OD-d₄) δ 8.36 (d, J = 2.5 Hz, 1H), 8.15 (d, J = 8.15 d = 8.1 |
| | Hz, 1H), 8.09 (d, J = 2.6 Hz, 1H), 7.60 (td, J = 8.9, 5.7 Hz, 1H), 7.25 (s, 2H), 7.15- |
| | 6.73 (m, 2H), 4.01 (s, 3H). |
| 2B | ¹H NMR (400 MHz, CD₃OD-d₄) δ 8.12 (d, J = 8.4 Hz, 1H), 7.71 (d, J = 8.4 Hz, 2H), |
| | 7.62-7.55 (m, 1H), 7.25 (s, 2H), 7.15-7.10 (m, 1H), 7.05 (d, J = 8.4 Hz, 2H), 7.00-6.98 |
| | (m, 1H), 3.88 (s, 3H). |
| 2C | ¹H NMR (400 MHz, CD₃OD-d₄) δ 8.15 (d, J = 8.0 Hz, 1H), 8.00 (s, 1H), 7.70 (s, |
| | 1H), 7.70-7.60 (m, 1H), 7.25 (s, 2H), 7.20-7.05 (m, 2H), 3.33 (s, 3H). |
| 2D | ¹H NMR (400 MHz, CD₃OD-d₄) δ. 8.15 (d, J = 8.0 Hz, 1H), 8.03 (s, 1H), 7.70 (s, 1H), |
| | 7.70-7.60 (m, 1H), 7.25 (s, 2H), 7.20-7.12 (m, 1H), 7.12-7.03 (m, 1H), 4.20 (q, 2H), |
| | 1.41 (t, 3H). |
| 2E | ¹H NMR (400 MHz, DMSO-d₆) δ 14.01 (s, 1H), 12.84 (s, 1H), 9.88 (s, 1H), 8.26 (d, J = |
| | 8.4 Hz, 1H), 7.55-7.50 (m, 1H), 7.35-7.25 (m, 2H), 7.12 (s, 1H), 7.10-7.02 (m, 1H), 3.67 |
| | (s, 3H), 2.17 (s, 3H), 2.09 (s, 3H). |
| 2F | ¹H NMR (400 MHz, CD₃OD-d₄) δ 8.13 (d, J = 8.4 Hz, 1H), 7.63-7.57 (m, 1H), 7.46- |
| | 7.42 (m, 1H), 7.34 (d, J = 8.4 Hz, 1H), 7.24-7.12 (m, 5H), 7.01-6.97 (m, 1H), 3.82 (s, |
| | 3H). |
| 2G | ¹H NMR (400 MHz, CD₃OD-d₄) δ 8.10 (d, J = 8.4 Hz, 1H), 7.68-7.56 (m, 3H), 7.40 |
| | (d, J = 8.0 Hz, 2H), 7.24 (s, 2H), 7.15-7.10 (m, 1H), 6.95-6.91 (m, 1H), 3.03-2.94 |
| | (m, 1H), 1.31-1.13 (m, 6H). |
| 2H | ¹H NMR (400 MHz, CD₃OD-d₄) δ 8.16 (d, J = 8.4 Hz, 1H), 8.05 (s, 1H), 7.59 (m, 1H), |
| | 7.25 (s, 2H), 7.13 (m, 2H), 3.86 (s, 3H). |
| 2I | ¹H NMR (400 MHz, CD₃OD-d₄) δ 8.16 (d, J = 8.4 Hz, 1H), 7.88 (s, 1H), 7.64-7.58 |
| | (m, 1H), 7.25 (s, 2H), 7.18-7.15 (m, 1H), 7.14-7.06 (m, 1H), 3.82 (s, 3H), 2.25 (s, |
| | 3H). |
| 2J | ¹H NMR (400 MHz, DMSO-d₆) δ 14.78 (s, 1H), 10.66 (s, 1H), 8.52 (d, J = 2.6 Hz, 1H), |
| | 8.23-8.16 (m, 2H), 7.77 (s, 2H), 7.50-7.05 (m, 4H), 3.98 (s, 3H). |
| 2K | ¹H NMR (400 MHz, CD₃OD-d₄) δ 8.06 (d, 1H), 7.77 (s, 1H), 7.73 (s, 2H), 7.61 (td, |
| | J = 8.8, 5.6 Hz, 1H), 7.32 (dd, J = 8.6, 5.6 Hz, 1H), 7.10 (m, 1H), 3.86 (s, 3H), 3.74 (s, |
| | 3H). |
| 2L | ¹H NMR (400 MHz, CD₃OD-d₄) δ 8.11 (d, J = 8.4 Hz, 1H), 7.77-7.54 (m, 3H), 7.48 |
| | (dd, 2H), 7.24 (s, 2H), 7.18-7.06 (m, 1H), 6.94 (t, J = 7.2 Hz, 1H), 2.95 (p, J = 6.9 Hz, |
| | 1H), 1.21 (d, J = 6.9 Hz, 6H). |
| 2M | ¹H NMR (400 MHz, CD₃OD-d₄) δ 8.33 (d, J = 2.4 Hz, 1H), 8.12-8.09 (m, 1H), 7.58- |
| | 7.54 (m, 1H), 7.29-7.23 (m, 4H), 7.12 (dd, J = 6 Hz, 8.4 Hz, 1H), 4.00 (s, 3H). |
| 2N | ¹H NMR (400 MHz, CD₃OD-d₄) δ 8.03 (m, 1H), 7.93 (d, J = 3.2 Hz, 1H), 7.74 (s, 2H), |
| | 7.62 (s, 1H), 7.34 (s, 2H), 3.82 (s, 3H), 2.24 (s, 3H). |
| 2O | ¹H NMR (400 MHz, CD₃OD-d₄) δ 8.35 (d, J = 2.6 Hz, 1H), 8.13 (dd, J = 2.6, 0.9 Hz, |
| | 1H), 8.01 (d, J = 8.5 Hz, 1H), 7.75-7.66 (m, 3H), 7.46 (t, J = 7.9 Hz, 1H), 7.32 (dd, |
| | J = 7.7, 1.6 Hz, 1H), 7.24 (dd, J = 8.4, 5.9 Hz, 1H), 3.94 (s, 3H). |
| 2P | ¹H NMR (400 MHz, CD₃OD-d₄) δ 8.39 (d, J = 2.6 Hz, 1H), 8.06 (d, J = 2.6 Hz, 1H), |
| | 8.00 (d, J = 8.4 Hz, 1H), 7.75 (s, 2H), 7.29-7.14 (m, 4H), 4.07 (s, 3H), 2.12 (d, J = 1.2 |
| | Hz, 3H). |
| 2Q | ¹H NMR (400 MHz, CD₃OD-d₄) δ 8.76 (s, 1H), 8.13 (t, J = 7.5 Hz, 2H), 7.50 (s, 1H), |
| | 7.47 (s, 1H), 7.24 (s, 2H), 7.13 (s, 1H), 7.01 (s, 1H), 5.09 (s, 2H). |
| 2R | ¹H NMR (400 MHz, CD₃OD-d₄) δ 8.12 (d, J = 8.4 Hz, 1H), 7.89 (d, J = 8.1 Hz, 1H), |
| | 7.66-7.43 (m, 3H), 7.34-7.22 (m, 3H), 7.11-6.98 (m, 2H), 3.96 (m, J = 6.7 Hz, |
| | 1H), 1.26 (d, J = 6.8 Hz, 6H). |
| 2S | ¹H NMR (400 MHz, CD₃OD-d₄) δ 8.05 (d, J = 8.6 Hz, 1H), 7.72 (s, 2H), 7.62-7.54 |
| | (m, 1H), 7.31 (d, J = 12.8 Hz, 2H), 7.18 (t, J = 8.8 Hz, 1H), 7.07 (s, 1H), 3.94 (s, 3H). |
| 2T | ¹H NMR (400 MHz, CD₃OD-d₄) δ 8.35 (d, J = 2.4 Hz, 1H), 8.10 (d, J = 2.8 Hz, 1H), |
| | 8.05 (d, J = 8.4 Hz, 1H), 7.75-7.69 (m, 3H), 7.35-7.30 (m, 1H), 7.25-7.21 (m, 1H), |
| | 3.95 (s, 3H). |
| 2U | ¹H NMR (400 MHz, CD₃OD-d₄) δ 8.36 (dd, J = 5.0, 1.9 Hz, 1H), 8.12 (dd, J = 7.6, 1.9 |
| | Hz, 1H), 8.03 (d, J = 8.4 Hz, 1H), 7.75 (s, 2H), 7.60 (td, J = 9.0, 5.8 Hz, 1H), 7.31- |
| | 7.22 (m, 1H), 7.18-7.03 (m, 2H), 4.03 (s, 3H). |
| 2V | ¹H NMR (400 MHz, CD₃OD-d₄) δ 8.29 (d, J = 3.0 Hz, 1H), 8.04 (d, J = 8.4 Hz, 1H), |
| | 7.96 (dd, J = 7.3, 3.0 Hz, 1H), 7.75 (d, J = 2.9 Hz, 2H), 7.62 (td, J = 8.9, 5.7 Hz, 1H), |
| | 7.34-7.25 (m, 1H), 7.22-7.12 (m, 1H), 4.01 (s, 3H). |
| 2W | ¹H NMR (400 MHz, CD₃OD-d₄) δ 8.38 (s, 1H), 8.15 (d, J = 8.4 Hz, 1H), 7.61 (td, J = |
| | 8.9, 5.7 Hz, 1H), 7.25 (s, 2H), 7.12 (ddd, J = 10.8, 7.5, 3.4 Hz, 2H), 4.16 (s, 3H). |
| 2X | ¹H NMR (400 MHz, CD₃OD-d₄) δ 8.02 (d, J = 12 Hz, 1H), 7.94 (s, 1H), 7.72 (s, |
| | 2H), 7.87-7.81 (m, 1H), 7.41-7.22 (m, 3H), 4.27 (s, 3H). |
| 2Y | ¹H NMR (400 MHz, CD₃OD-d₄) δ 8.06 (d, J = 8.6 Hz, 1H), 7.98 (s, 1H), 7.75 (s, 2H), |
| | 7.64 (td, J = 8.9, 5.7 Hz, 1H), 7.40-7.29 (m, 1H), 7.28-7.09 (m, 1H), 4.27 (s, 3H). |
| 2Z | ¹H NMR (400 MHz, CD₃OD-d₄) δ 8.35 (s, 1H), 8.10-8.05. (m, 2H), 7.70-7.50. (m, |
| | 1H), 7.25-7.10. (m, 2H), 4.00 (s, 3H). |
| 2AA | ¹H NMR (400 MHz, CD₃OD-d₄) δ 8.13 (d, J = 8.4 Hz, 1H), 8.02 (d, J = 8.1 Hz, 1H), |
| | 7.56 (td, J = 8.9, 5.7 Hz, 1H), 7.27-7.19 (m, 3H), 7.11 (td, J = 9.0, 1.8 Hz, 1H), 6.99 |
| | (dd, J = 8.4, 5.7 Hz, 1H), 2.83 (s, 3H), 2.57 (s, 3H). |
| 2AB | ¹H NMR (400 MHz, CD₃OD-d₄) δ 8.56 (d, J = 1 Hz, 1H), 8.01 (d, J = 8.8 Hz, 1H), |
| | 7.93 (dd, J = 2.8 Hz, 8 Hz, 1H), 7.74 (s, 2H), 7.64-7.58 (m, 1H), 7.26-7.17 (m, 2H), |
| | 2.81 (s, 3H). |
| 2AC | ¹H NMR (400 MHz, CD₃OD-d₄) δ 8.18-8.09 (m, 2H), 7.72 (dd, J = 6.4, 2.2 Hz, 1H), |
| | 7.57 (td, J = 8.9, 5.7 Hz, 1H), 7.25 (s, 2H), 7.16-7.06 (m, 2H), 6.44 (t, J = 6.8 Hz, |
| | 1H). |
| 2AD | ¹H NMR (400 MHz, CD₃OD-d₄) δ 8.27 (d, J = 2.6 Hz, 1H), 8.16 (dd, J = 5.4, 2.9 Hz, |
| | 2H), 7.68-7.53 (m, 2H), 7.30-7.13 (m, 4H), 3.92 (s, 3H). |
| 2AE | ¹H NMR (400 MHz, CD₃OD-d₄) δ 8.07 (dd, J = 13.0, 8.5 Hz, 1H), 7.77 (d, J = 1.3 Hz, |
| | 2H), 7.75-7.60 (m, 1H), 7.51-7.38 (m, 1H), 7.30-7.11 (m, 1H), 2.43-1.86 (m, |
| | 3H). |
| 2AF | ¹H NMR (400 MHz, CD₃OD-d₄) δ 8.14 (d, J = 8.4 Hz, 1H), 7.95-7.74 (m, 2H), 7.64- |
| | 7.40 (m, 1H), 7.25 (s, 2H), 7.16-6.94 (m, 2H), 4.74 (s, 2H). |
| 2AG | ¹H NMR (400 MHz, CD₃OD-d₄) δ 8.03 (d, J = 12 Hz, 1H), 7.76 (s, 1H), 7.83-7.56 (m, |
| | 3H), 7.34-7.15 (m, 2H), 7.20-7.16 (t, J = 8 Hz, 1H). |
| 2AH | ¹H NMR (400 MHz, CD₃OD-d₄) δ 8.42 (s, 1H), 8.11 (d, J = 8 Hz, 1H), 7.97 (s, 1H), |
| | 7.55 (m, 1H), 7.23 (m, 2H), 7.11 (t, J = 8 Hz, 1H), 7.00 (m, 1H), 2.8 (s, 3H), 2.33 (s, |
| | 3H). |
| 2AI | ¹H NMR (400 MHz, CD3OD-d₄) δ 8.12 (d, J = 8.4 Hz, 1H), 7.72 (d, J = 7.6 Hz, 1H), |
| | 7.53-7.46 (m, 3H), 7.41 (d, J = 7.2 Hz, 1H), 7.24 (s, 2H), 7.19-7.14 (m, 1H), 7.07- |
| | 6.96 (m, 2H), 2.61 (s, 3H), 2.34 (s, 3H). |
| 2AJ | ¹H NMR (400 MHz, CD₃OD-d₄) δ 8.13 (d, J = 2.8 Hz, 1H), 8.04 (d, J = 2.8 Hz, |
| | 1H), 8.01 (d, J = 8.4 Hz, 1H), 7.76 (s, 2H), 7.60 (m, 1H), 7.34 (m, 1H), 7.11 |
| | (m), 6.44 (t, 1H), 3.61 (s, 3H). |
| 2AK | ¹H NMR (400 MHz, CD₃OD-d₄) δ 8.56 (d, J = 1.4 Hz, 1H), 8.48-8.39 (m, 2H), 8.10 |
| | (d, J = 8.6 Hz, 1H), 7.77 (s, 2H), 7.63 (dd, J = 8.6, 5.7 Hz, 1H), 4.06 (s, 3H). |
| 2AL | ¹H NMR (400 MHz, CD₃OD-d₄) δ 8.02 (d, J = 8 Hz, 1H), 7.78 (d, J = 8 Hz, 1H), 7.74 |
| | (s, 2H), 7.66-7.60 (m, 1H), 7.35-7.29 (m, 2H), 7.15-7.10 (t, J = 8 Hz, 1H), 6.85 (d, J = 8 |
| | Hz, 1H). |
| 2AM | ¹H NMR (400 MHz, CD₃OD-d₄) δ 8.03 (d, J = 12 Hz, 1H), 7.83 (d, J = 8 Hz, 1H), |
| | 7.78-7.70 (m, 3H), 7.54 (d, J = 4 Hz, 1H), 7.28 (t, J = 6 Hz, 1H), 7.13 (t, J = 10 Hz, |
| | 1H), 6.98 (d, J = 4 Hz, 1H). |
| 2AN | ¹H NMR (400 MHz, CD₃OD-d₄) δ 8.16 (d, J = 2.8 Hz, 1H), 8.07-8.03 (m, 2H), 7.73 (s, |
| | 2H), 7.60-7.54 (m, 1H), 7.34 (dd, J = 6 Hz, 8.8 Hz, 1H), 7.13 (t, J = 9.2 Hz, 1H), 3.59 |
| | (s, 3H). |
| 2AO | ¹H NMR (400 MHz, CD₃OD-d₄) δ 8.35 (d, J = 4 Hz, 1H), 8.12 (d, J = 4 Hz, 1H), 7.54 |
| | (m, 3H), 7.43 (t, J = 8 Hz, 1H), 3.98 (s, 3H), 2.53 (s, 3H). |
| 2AP | ¹H NMR (400 MHz, CD₃OD-d₄) δ 8.03 (d, J = 8.4 Hz, 1H), 7.71 (m, 2H), 7.53-7.58 |
| | (m, 2H), 7.45 (m, 1H), 7.27 (m, 1H), 7.06-7.20 (m, 2H), 4.05 (s, 3H). |
| 2AQ | ¹H NMR (400 MHz, CD₃OD-d₄) δ 8.02 (d, J = 8.4 Hz, 1H), 7.74 (s, 2H), 7.58 (m, 1H), |
| | 7.52 (dd, 1H), 7.48 (m, 1H), 7.39 (m, 1H), 7.27 (m, 1H), 7.14 (m, 1H), 6.89 (m, 1H). |
| 2AR | ¹H NMR (400 MHz, CD₃OD-d₄) δ 8.02 (d, J = 8.4 Hz, 1H), 7.74 (s, 2H), 7.47-7.53 (m, |
| | 2H), 7.20-7.35 (m, 3H), 7.09 (m, 1H), 3.90 (s, 3H), 2.52 (s, 3H). |
| 2AS | ¹H NMR (400 MHz, CD₃OD-d₄) δ 8.32 (s, 1H), 8.08 (s, 1H), 7.84 (d, 1H), 7.56 (m, |
| | 1H), 7.07-7.13 (m, 2H), 3.99 (s, 3H), 3.44 (s, 3H), 3.15 (s, 3H). |
| 2AT | ¹H NMR (400 MHz, DMSO-d₆) δ 13.97 (1H, s) 12.80 (1H, br s) 10.47 (1H, br s) 8.21 |
| | (1H, d, J = 8.41 Hz) 7.46-7.58 (1H, m) 7.32-7.45 (1H, m) 7.07-7.31 (5H, m) 6.92- |
| | 7.05 (1H, m) 2.46 (s, 3H). |
| 2AU | ¹H NMR (400 MHz, CD₃OD-d₄) δ 8.03 (d, J = 8.4 Hz, 1H), 7.70-7.73 (m, 2H), 7.48 |
| | (m, 1H), 7.45 (m, 1H), 7.26 (m, 1H), 7.01-7.13 (m, 3H), 2.51 (s, 3H). |
| 2AV | ¹H NMR (400 MHz, DMSO-d₆) δ 13.38 (1H, s), 12.55-12.81 (1H, m), 10.54-10.76 |
| | (1H, m), 8.33-8.50 (1H, m), 7.84 (1H, d, J = 2.15 Hz), 7.66-7.80 (2H, m), 7.50 (1H, |
| | s), 7.31 (1H, td, J = 8.71, 5.87 Hz), 7.13-7.25 (2H, m), 6.94-7.13 (2H, m). |
| 2AW | ¹H NMR (400 MHz, DMSO-d₆) δ 13.37 (1H, s), 12.60-12.77 (1H, m), 10.41 (1H, br |
| | s), 8.39 (1H, d, J = 8.41 Hz), 7.59-7.67 (2H, m), 7.38-7.49 (2H, m), 7.07-7.34 (4H, |
| | m), 7.02 (1H, br d, J = 9.00 Hz), 2.54 (3H, s). |
| 2AX | ¹H NMR (400 MHz, DMSO-d₆) δ 14.01 (1H, s), 10.69 (1H, br d, J = 1.76 Hz), 8.13- |
| | 8.26 (1H, m), 7.84 (1H, d, J = 2.35 Hz), 7.68-7.80 (2H, m), 7.41 (1H, td, J = |
| | 8.90, 5.87 Hz), 7.13-7.29 (3H, m), 7.06 (1H, br dd, J = 8.02, 5.87 Hz). |
| 2AY | ¹H NMR (400 MHz, DMSO-d₆) δ 13.96 (1H, s), 12.78 (2H, br s), 10.51 (2H, br s), 8.21 |
| | (2H, d, J = 8.41 Hz), 7.74 (2H, br d, J = 7.83 Hz), 7.36 (2H, br t, J = 8.02 Hz), 7.06- |
| | 7.28 (4H, m), 6.99 (1H, br dd, J = 8.02, 5.87 Hz), 5.73 (1H, s), 2.60-2.68 (3H, s). |
| 2BA | ¹H NMR (400 MHz, DMSO-d₆) δ 13.96 (1H, s), 12.79 (2H, br s), 10.50 (2H, br s), |
| | 8.22 (1H, d, J = 8.41 Hz), 7.57 (1H, d, J = 8.80 Hz), 7.30-7.46 (3H, m), 7.09- |
| | 7.28 (5H, m), 7.04 (1H, br dd, J = 8.12, 5.77 Hz), 5.73 (1H, s), 3.76 (3H, s). |
| 2BB | ¹H NMR (400 MHz, DMSO-d₆) δ 13.96 (1H, s), 13.76 (1H, br s), 12.63-12.92 (2H, m), |
| | 10.75 (2H, br d, J = 9.59 Hz), 8.05-8.32 (3H, m), 7.79-8.01 (2H, m), 7.62-7.79 (2H, |
| | m), 7.49-7.61 (1H, m), 7.34-7.46 (2H, m), 7.06-7.29 (4H, m), 6.98-7.05 (1H, m). |
| 2BD | ¹H NMR (400 MHz, DMSO-d₆) δ 10.77 (1H, br s), 8.17 (1H, d, J = 8.61 Hz), 7.76-7.85 |
| | (1H, m), 7.60-7.72 (3H, m), 7.39-7.57 (2H, m), 7.25-7.36 (1H, m) 7.13-7.25 (1H, m). |
| 2BE | ¹H NMR (400 MHz, DMSO-d₆) δ 10.71 (1H, br s) 8.16 (1H, d, J = 8.41 Hz) 7.59-7.72 |
| | (4H, m) 7.50-7.59 (1H, m) 7.40 (1H, td, J = 8.80, 5.87 Hz) 7.13-7.34 (2H, m). |
| 2BF | ¹H NMR (400 MHz, DMSO-d₆) δ 9.92 (1H, s), 8.15 (1H, d, J = 8.61 Hz), 7.56-7.75 |
| | (4H, m), 7.31-7.48 (1H, m), 7.12-7.28 (3H, m), 6.95-7.06 (1H, m), 3.80 (3H, s). |
| 2BG | ¹H NMR (400 MHz, DMSO-d₆) δ 10.88 (1H, br s), 8.16 (1H, d, J = 8.41 Hz), 7.61-7.81 |
| | (3H, m), 7.46 (1H, td, J = 8.90, 5.87 Hz), 7.17-7.37 (4H, m). |
| 2BH | ¹H NMR (400 MHz, DMSO-d₆) δ 10.76 (1H, br s), 8.16 (1H, d, J = 8.41 Hz), 7.73 (2H, |
| | s), 7.40-7.68 (4H, m), 7.09-7.36 (2H, m). |
| 2BI | ¹H NMR (400 MHz, DMSO-d₆) δ 10.24 (1H, s), 8.15 (1H, d, J = 8.61 Hz), 7.68 (2H, s), |
| | 7.53 (1H, s), 7.14-7.39 (5H, m), 2.51 (3H, s), 2.26 (3H, s). |
| 2BJ | ¹H NMR (400 MHz, DMSO-d₆) δ 10.48 (1H, s), 8.15 (1H, d, J = 8.61 Hz), 7.69 (2H, s), |
| | 7.34-7.56 (4H, m), 7.24-7.33 (1H, m), 7.19 (1H, br dd, J = 8.12, 5.97 Hz), 2.54 (3H, s). |
| 2BK | ¹H NMR (400 MHz, DMSO-d₆) δ 10.62-10.76 (1H, m), 8.16 (1H, d, J = 8.41 Hz), 8.08 |
| | (1H, dd, J = 7.53, 1.27 Hz), 7.75-8.01 (3 H, m), 7.52-7.75 (2H, m), 7.24-7.51 (3H, m), |
| | 7.17 (1H, br dd, J = 7.53, 5.18 Hz). |
| 2BL | ¹H NMR (400 MHz, DMSO-d₆) δ 10.70 (1H, br s), 8.16 (1H, d, J = 8.41 Hz), 7.92 (2H, |
| | ddd, J = 19.32, 7.97, 1.47 Hz), 7.69 (2H, br s), 7.52 (1H, t, J = 8.02 Hz), 7.40 (1H, td, |
| | J = 8.90, 5.87 Hz), 7.14-7.33 (2H, m). |
| 2BM | ¹H NMR (400 MHz, DMSO-d₆) δ 10.80 (1H, br s), 8.63 (1H, dd, J = 4.79, 1.86 Hz), |
| | 8.29 (1H, dd, J = 7.82, 1.76 Hz), 8.15 (1H, d, J = 8.41 Hz), 7.73 (2H, s), 7.60 (1H, dd, |
| | J = 7.82, 4.70 Hz), 7.45 (1H, td, J = 8.85, 5.97 Hz), 7.17-7.36 (2H, m). |
| 2BN | ¹H NMR (400 MHz, DMSO-d₆) δ 10.63 (1H, br s), 8.16 (1H, d, J = 8.41 Hz), 7.84-7.94 |
| | (2H, m), 7.70 (2H, br s), 7.59 (1H, dd, J = 8.51, 2.05 Hz), 7.39 (1H, td, J = 8.80, 5.87 |
| | Hz), 7.16-7.32 (2H, m). |
| 2BO | ¹H NMR (400 MHz, DMSO-d₆) δ 10.45 (1H, br s), 8.16 (1H, d, J = 8.41 Hz), 7.56-7.82 |
| | (5H, m), 7.40 (1H, td, J = 8.85, 5.97 Hz), 7.15-7.34 (2H, m). |
| 2BP | ¹H NMR (400 MHz, DMSO-d₆) δ 10.50 (1H, s), 8.16 (1H, d, J = 8.41 Hz), 7.90 (1H, |
| | dd, J = 7.82, 1.37 Hz), 7.57-7.73 (4H, m), 7.44-7.53 (1H, m), 7.38 (1H, td, J = 8.71, |
| | 6.06 Hz), 7.12-7.29 (2H, m). |
| 2BQ | ¹H NMR (400 MHz, DMSO-d₆) δ 10.53 (1H, br s) 8.08-8.23 (3H, m), 8.02 (1H, dt, J = |
| | 8.31, 1.32 Hz), 7.80 (1H, t, J = 7.92 Hz), 7.70 (2H, br s), 7.40 (1H, td, J = 8.90, 5.87 |
| | Hz), 7.14-7.34 (2H, m). |
| 2BS | ¹H NMR (400 MHz, DMSO-d₆) δ 10.56 (1H, s), 8.16 (1H, d, J = 8.41 Hz), 7.94 (1H, |
| | dd, J = 8.90, 5.97 Hz), 7.59-7.78 (3H, m), 7.32-7.45 (2H, m), 7.12-7.32 (2H, m). |
| 2BT | ¹H NMR (400 MHz, DMSO-d₆) δ 10.57 (1H, s), 8.15 (1H, d, J = 8.41 Hz), 7.58-7.81 |
| | (4H, m), 7.25-7.47 (4H, m), 7.18 (1H, dd, J = 8.02, 5.87 Hz). |
| 2BU | ¹H NMR (400 MHz, DMSO-d₆) δ 10.35 (1H, s), 8.16 (1H, d, J = 8.41 Hz), 7.73 (2H, s), |
| | 7.68 (1H, t, J = 60 Hz), 7.47 (1H, dt, J = 8.0, 4.0), 7.32 (1H, t, J = 8.0 Hz), 7.19 (1H, br |
| | dd, J = 8.12, 5.97 Hz), 2.26 (3H, s). |
| 2BV | ¹H NMR (400 MHz, DMSO-d₆) δ 10.28 (1H, s), 8.15 (1H, d, J = 8.61 Hz), 7.49-7.78 |
| | (7H, m), 7.37 (1H, td, J = 8.80, 5.87 Hz), 7.09-7.31 (2H, m). |
| 2BW | ¹H NMR (400 MHz, DMSO-d₆) δ 10.33-10.59 (1H, bs), 8.16 (1H, br t, J = 9.29 |
| | Hz), 7.57-7.85 (5H, m), 7.23-7.47 (2H, m), 7.17 (1H, br d, J = 5.67 Hz), 4.74- |
| | 4.93 (2H, m). |
| 2BX | ¹H NMR (400 MHz, DMSO-d₆) δ 13.18 (1H, bs), 10.63 (1H, s), 8.12 (1H, s), 7.79-7.90 |
| | (2H, m), 7.68-7.79 (2H, m), 7.46 (1H, s), 7.15-7.36 (2H, m), 6.93-7.01 (1H, m). |
| 2BY | ¹H NMR (400 MHz, DMSO-d₆) δ 10.36 (1H, s), 8.48 (1H, d, J = 2.54 Hz), 8.12 (1H, d, |
| | J = 0.98 Hz), 8.04 (1H, d, J = 2.54 Hz), 7.83 (1H, d, J = 8.41 Hz), 7.46 (1H, s), 7.26- |
| | 7.39 (1H, m), 7.19 (1H, td, J = 9.10, 1.37 Hz), 6.99 (1H, dd, J = 8.22, 0.98 Hz), 3.89 (s, |
| | 3H). |
| 2CA | ¹H NMR (400 MHz, DMSO-d₆) δ 11.59 (1H, br s), 8.41 (2H, dd, J = 16.43, 2.54 Hz), |
| | 8.32 (1H, d, J = 8.80 Hz), 8.09 (1H, s), 7.68-7.86 (4H, m), 7.01 (1H, dd, J = 8.80, 2.74 |
| | Hz), 3.92 (3H, s). |
| 2CB | ¹H NMR (400 MHz, DMSO-d₆) δ 8.46 (1H, br d, J = 2.35 Hz), 8.39 (1H, d, J = 8.61 |
| | Hz), 8.25 (2H, br d, J = 2.35 Hz), 8.11 (1H, s), 7.82 (2H, s), 7.74 (1H, br d, J = 8.41 |
| | Hz), 7.54 (1H, br t, J = 6.16 Hz), 3.87 (3H, s). |
| 2CC | ¹H NMR (400 MHz, DMSO-d₆) δ 12.75 (1H, s), 10.65 (1H, s), 7.76 (1H, br d, J = 8.02 |
| | Hz), 7.44-7.66 (3H, m), 7.25-7.39 (2H, m), 7.14-7.25 (1H, m), 6.92 (1H, br d, J = 8.02 |
| | Hz), 3.30 (3H, s). |
| 2CD | ¹H NMR (400 MHz, DMSO-d₆) δ 10.44 (1H, s) 8.72 (1H, br d, J = 4.50 Hz) 8.33-8.56 |
| | (2H, m), 8.19 (1H, br d, J = 8.02 Hz), 8.00-8.12 (1H, m), 7.91 (1H, br t, J = 7.63 Hz), |
| | 7.33-7.51 (2H, m), 7.27 (1H, br t, J = 8.90 Hz), 7.09 (1H, br t, J = 6.94 Hz), 3.89 (3H, |
| | s). |
| 2CE | ¹H NMR (400 MHz, CD₃OD-d₄) δ 8.80 (2H, d), 8.17 (s, 1H), 8.04 (d, 1H), 7.70 (s, |
| | 2H), 7.65 (m, 1H), 7.22-7.37 (m, 2H). |
| 2CF | ¹H NMR (400 MHz, CD₃OD-d₄) δ 8.33 (s, 1H), 8.09-8.06 (m, 2H), 7.58 (m, 1H), 7.07- |
| | 7.13 (m, 2H), 4.01 (s, 3H), 3.63 (t, 2H), 2.71 (t, 2H), 2.42 (s, 6H). |
| 2CG | ¹H NMR (400 MHz, CD₃OD-d₄) δ 8.30 (s, 1H), 8.09-8.05 (m, 2H), 7.52 (m, 1H), 7.04- |
| | 7.14 (m, 2H), 3.98 (s, 3H), 3.49 (t, 2H), 2.59 (t, 2H), 2.40 (s, 6H), 1.93 (m, 2H). |
| 2CH | ¹H NMR (400 MHz, CD₃OD-d₄) δ 8.36 (s, 1H), 8.09-8.07 (m, 2H), 7.60 (m, 1H), 7.08- |
| | 7.16 (m, 2H), 4.00 (s, 3H), 3.77 (t, 2H), 3.60 (t, 2H). |
| 2CI | ¹H NMR (400 MHz, CD₃OD-d₄) δ 8.36 (s, 1H), 8.09-8.07 (m, 2H), 7.60 (m, 1H), 7.08- |
| | 7.16 (m, 2H), 4.00 (s, 3H), 3.71 (t, 2H), 3.60 (t, 2H), 1.89 (m, 2H). |
| 2CJ | ¹H NMR (400 MHz, CD₃OD-d₄) δ 8.35 (s, 1H), 8.08-8.06 (m, 2H), 7.60 (m, 1H), 7.08- |
| | 7.15 (m, 2H), 4.00 (s, 3H), 3.28 (d, 2H), 1.97 (m, 1H), 1.02 (d, 6H). |
| 2CK | ¹H NMR (400 MHz, CD₃OD-d₄) δ 8.35 (s, 1H), 8.09-8.06 (m, 2H), 7.58 (m, 1H), 7.08- |
| | 7.15 (m, 2H), 4.30 (m, 1H), 4.00 (s, 3H), 1.33 (d, 6H). |
| 2CL | ¹H NMR (400 MHz, CD₃OD-d₄) δ 8.30-8.33 (m, 2H), 8.20 (d, 1H), 7.96 (s, 1H), 7.25 |
| | (s, 2H), 7.23 (m, 1H), 7.11 (m, 1H), 4.03 (s, 3H). |
| 2CM | ¹H NMR (400 MHz, CD₃OD-d₄) δ 8.64 (s, 1H), 8.13 (s, 1H), 8.04 (m, 1H), 7.72 (s, |
| | 2H), 7.63 (m, 1H), 7.19-7.35 (m, 2H), 2.83 (s, 3H). |

### Example 10—Synthesis of N-(2,4-Difluoro-3-(3-phenyl-1H-indazol-6-yl)phenyl)-2,5-difluorobenzenesulfonamide

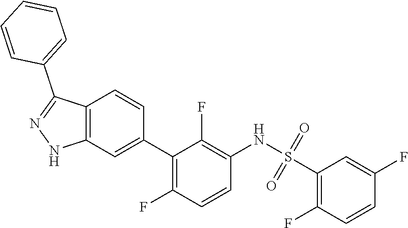 [SMILES: O=S(=O)(Nc1ccc(F)c(-c2ccc3c(-c4ccccc4)n[nH]c3c2)c1F)c1cc(F)ccc1F]

### Part I—Synthesis of N-(2,4-Difluoro-3-(3-iodo-1H-indazol-6-yl)phenyl)-2,5-difluorobenzenesulfonamide

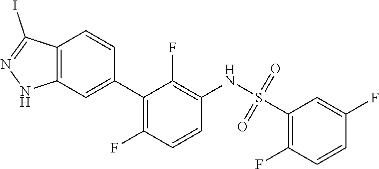 [SMILES: O=S(=O)(Nc1ccc(F)c(-c2ccc3c(I)n[nH]c3c2)c1F)c1cc(F)ccc1F]

A mixture of N-(2,4-difluoro-3-(1H-indazol-6-yl)phenyl)-2,5-difluorobenzenesulfonamide (1.35 g, 3.2 mmol), iodine (894 mg, 3.52 mmol), DMF (16 mL), and powdered potassium hydroxide (720 mg, 12.82 mmol) was stirred at room temperature overnight. The mixture was diluted with water, and partitioned between ethyl acetate and aqueous sodium bisulfite. The organic layer was washed with brine, dried (Na₂SO₄), and concentrated. The residue was purified via MPLC to afford N-(2,4-difluoro-3-(3-iodo-1H-indazol-6-yl)phenyl)-2,5-difluorobenzenesulfonamide (980 mg, 56%).

### Part II—Synthesis of N-(2,4-Difluoro-3-(3-iodo-1-((2-(trimethylsilyl)ethoxy)methyl)-1H-indazol-6-yl)phenyl)-2,5-difluoro-N-((2-(trimethylsilyl)ethoxy)methyl)benzenesulfonamide

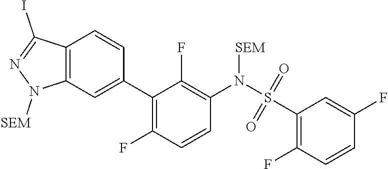 [SMILES: C[Si](C)(C)CCOCN(c1ccc(F)c(-c2ccc3c(I)nn(COCC[Si](C)(C)C)c3c2)c1F)S(=O)(=O)c1cc(F)ccc1F]

Sodium hydride (60% , 206 mg, 5.37 mmol) was added to a stirred solution of N-(2,4-difluoro-3-(3-iodo-1H-indazol-6-yl)phenyl)-2,5-difluorobenzenesulfonamide (980 mg, 1.79 mmol) in THF (18 mL) at 0° C. The mixture was stirred for an hour, and 2-(trimethylsilyl)ethoxymethyl chloride (896 mg, 5.37 mmol) was added. The ice bath was removed after thirty minutes, and the reaction was stirred overnight at room temperature. Water was added and the mixture was extracted with ethyl acetate. The organic layer was dried (Na₂SO₄), concentrated, and the residue was purified by MPLC eluting with 20% ethyl acetate in hexanes to afford N-(2,4-difluoro-3-(3-iodo-1-((2-(trimethylsilyl)ethoxy)methyl)-1H-indazol-6-yl)phenyl)-2,5-difluoro-N-((2-(trimethylsilyl)ethoxy)methyl)benzenesulfonamide (900 mg, 62%).

### Part III—Synthesis of N-(2,4-Difluoro-3-(3-phenyl-1-((2-(trimethylsilyl)ethoxy)-methyl)-1H-indazol-6-yl)phenyl)-2,5-difluoro-N-((2-(trimethylsilyl)ethoxy)methyl)-benzenesulfonamide

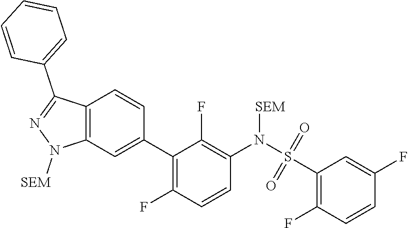 [SMILES: C[Si](C)(C)CCOCN(c1ccc(F)c(-c2ccc3c(-c4ccccc4)nn(COCC[Si](C)(C)C)c3c2)c1F)S(=O)(=O)c1cc(F)ccc1F]

A mixture of N-(2,4-difluoro-3-(3-iodo-1-((2-(trimethylsilyl)ethoxy)methyl)-1H-indazol-6-yl)phenyl)-2,5-difluoro-N-((2-(trimethylsilyl)ethoxy)methyl)benzenesulfonamide (100 mg, 0.12 mmol), potassium carbonate (34 mg, 0.25 mmol), phenylboronic acid (17 mg, 0.14 mmol) in toluene (3 mL), ethanol (1 mL), water (1 mL), and tetrakistriphenylphospine palladium (14 mg, 0.012 mmol) was heated at reflux overnight. The mixture was partitioned between ethyl acetate and water, and the organic phase was dried (Na₂SO₄), and concentrated. The residue was purified by MPLC eluting with 40% ethyl acetate in hexanes to afford N-(2,4-difluoro-3-(3-phenyl-1-((2-(trimethylsilyl)ethoxy)methyl)-1H-indazol-6-yl)phenyl)-2,5-difluoro-N-((2-(trimethylsilyl)ethoxy)methyl)benzenesulfonamide (33 mg, 35%).

### Part IV—Synthesis of N-(2,4-Difluoro-3-(3-phenyl-1H-indazol-6-yl)phenyl)-2,5-difluorobenzenesulfonamide

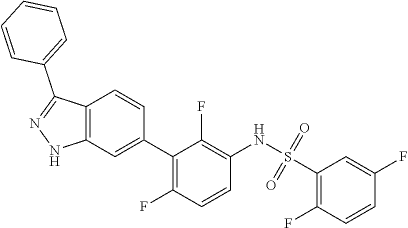 [SMILES: O=S(=O)(Nc1ccc(F)c(-c2ccc3c(-c4ccccc4)n[nH]c3c2)c1F)c1cc(F)ccc1F]

N-(2,4-Difluoro-3-(3-phenyl-1H-indazol-6-yl)phenyl)-2,5-difluorobenzenesulfonamide was prepared from N-(2,4-difluoro-3-(3-phenyl-1-((2-(trimethylsilyl)ethoxy)methyl)-1H-indazol-6-yl)phenyl)-2,5-difluoro-N-((2-(trimethylsilyl)ethoxy)methyl)benzenesulfonamide using the procedures described in Part III of Example 6. ¹H NMR (400 MHz, CD₃OD-d₄) δ 8.23 (s, 1H), 8.20 (m, 1H), 8.14 (d, 1H), 7.98 (s, 1H), 7.26 (s, 2H), 7.23 (m, 1H), 4.01 (s, 3H). (ES, m/z): (M+H)⁺ 498.

### Example 11—Synthesis of 5-Chloro-N-(3-fluoro-4-(7-fluoro-3-(1H-imidazol-2-yl)-1H-indazol-6-yl)pyridin-2-yl)-2-methoxypyridine-3-sulfonamide

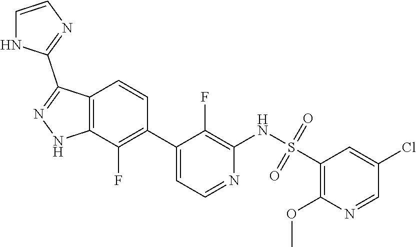 [SMILES: COc1ncc(Cl)cc1S(=O)(=O)Nc1nccc(-c2ccc3c(-c4ncc[nH]4)n[nH]c3c2F)c1F]

### Part I—Synthesis of 3-Fluoro-4-iodopyridin-2-amine

A 2.5 M solution of n-butyllithium in hexanes (23 mL, 57.6 mmol) was added slowly to a stirred solution of 2-amino-3-fluoropyridine (2.59 g, 23.0 mmol) in THE (100 mL) at −78° C. The mixture was stirred at −78° C. for ninety minutes. Iodine (17.56 g, 69.2 mmol) was added, and the reaction was stirred at −78° C. for an additional fifteen minutes, and then allowed to warm to room temperature. The mixture was quenched with a saturated solution of sodium thiosulfate, and was extracted with ethyl acetate. The organic layer was washed with brine, dried (Na₂SO₄), and concentrated. The residue was purified by MPLC, eluting with a gradient of 0-10% methanol in dichloromethane to afford 3-fluoro-4-iodopyridin-2-amine (1.15 g, 21%).

### Part II—Synthesis of 5-Chloro-N-(3-fluoro-4-(7-fluoro-3-(1H-imidazol-2-yl)-1H-indazol-6-yl)pyridin-2-yl)-2-methoxypyridine-3-sulfonamide

5-Chloro-N-(3-fluoro-4-(7-fluoro-3-(1H-imidazol-2-yl)-1H-indazol-6-yl)pyridin-2-yl)-2-methoxypyridine-3-sulfonamide was prepared from 3-fluoro-4-iodopyridin-2-amine using procedures similar to those described in Example 6. ¹H NMR (400 MHz, DMSO-d₆) δ 8.51 (d, J=2.35 Hz, 1H), 8.21-8.36 (m, 2H), 8.05-8.21 (m, 2H), 7.68 (s, 2H), 7.41-7.60 (m, 2H), 3.92 (s, 3H). (ES, m/z): (M+H)⁺ 518.

### Example 12—Synthesis of 5-Fluoro-N-(2-fluoro-3-(7-fluoro-3-(5-oxopyrrolidin-2-yl)-1H-indazol-6-yl)phenyl)-2-methoxypyridine-3-sulfonamide

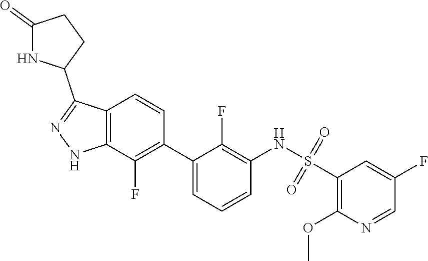 [SMILES: COc1ncc(F)cc1S(=O)(=O)Nc1cccc(-c2ccc3c(C4CCC(=O)N4)n[nH]c3c2F)c1F]

### Part I—Synthesis of Ethyl 5-oxo-1-((2-(trimethylsilyl)ethoxy)methyl)pyrrolidine-2-carboxylate

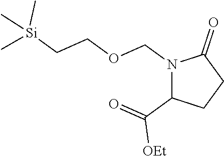 [SMILES: CCOC(=O)C1CCC(=O)N1COCC[Si](C)(C)C]

Sodium hydride (1.07 g, 28 mmol) was added to a stirred solution of L-Pyroglutamic acid ethyl ester (4.0 g, 25.5 mmol) in THE (128 mL) in an ice bath. The mixture was stirred an hour, and 2-(trimethylsilyl)ethoxymethyl chloride (4.67 g, 28 mmol) was added. The mixture was stirred for six hour, quenched with water, and extracted with ethyl acetate. The organic layer was then dried (Na₂SO₄), and concentrated. The residue was purified via MPLC eluting with 40% ethyl acetate in hexane to afford ethyl 5-oxo-1-((2-(trimethylsilyl)ethoxy)-methyl)pyrrolidine-2-carboxylate (4.17 g, 57%).

### Part II—Synthesis of 5-Oxo-1-((2-(trimethylsilyl)ethoxy)methyl)pyrrolidine-2-carboxylic acid

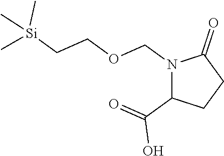 [SMILES: C[Si](C)(C)CCOCN1C(=O)CCC1C(=O)O]

A mixture of ethyl 5-oxo-1-((2-(trimethylsilyl)ethoxy)methyl)pyrrolidine-2-carboxylate (4.17 g, 14.5 mmol), ethanol (116 mL), and an aqueous solution of sodium hydroxide (1.16 g in 39 mL of water) was stirred at room temperature an hour. The ethanol was removed in vacuo, and the pH of the remaining aqueous mixture was adjusted to 2 with 6 N HCl. The mixture was extracted twice with ethyl acetate. The combined organic layers were then washed with brine, dried (Na₂SO₄), and concentrated to afford 5-oxo-1-((2-(trimethylsilyl)ethoxy)methyl)pyrrolidine-2-carboxylic acid (3.76 g, 100%) as a clear oil.

### Part III—Synthesis of N-Methoxy-N-methyl-5-oxo-1-((2-(trimethylsilyl)ethoxy)methyl)-pyrrolidine-2-carboxamide

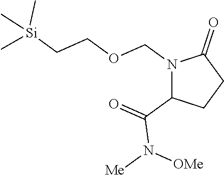 [SMILES: CON(C)C(=O)C1CCC(=O)N1COCC[Si](C)(C)C]

A mixture of 5-oxo-1-((2-(trimethylsilyl)ethoxy)methyl)pyrrolidine-2-carboxylic acid (3.76 g, 14.5 mmol), diisopropylethylamine (7.49 g, 58 mmol), 1-[bis(dimethylamino)methylene]-1H-1,2,3-triazolo[4,5-b]pyridinium 3-oxide hexafluorophosphate, N-[(dimethylamino)-1H-1,2,3-triazolo-[4,5-b]pyridin-1-ylmethylene]-N-methylmethanaminium hexafluorophosphate N-oxide (5.51 g, 14.5 mmol), and DMF (71 mL) was stirred for ten minutes. N,O-Dimethylhydroxylamine hydrochloride (2.83 g, 29 mmol) was added, and the reaction was stirred at room temperature overnight and concentrated. The residue was partitioned between ethyl acetate and water. The organic layer was washed with water, brine, dried (Na₂SO₄), and concentrated. The residue was purified via MPLC eluting with a gradient of 0-10% methanol in dichloromethane to afford N-methoxy-N-methyl-5-oxo-1-((2-(trimethylsilyl)ethoxy)methyl)pyrrolidine-2-carboxamide (2.94 g, 67%).

### Part IV—Synthesis of 5-(4-Bromo-2,3-difluorobenzoyl)-1-((2-(trimethylsilyl)ethoxy)-methyl)pyrrolidin-2-one

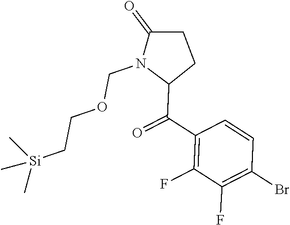 [SMILES: C[Si](C)(C)CCOCN1C(=O)CCC1C(=O)c1ccc(Br)c(F)c1F]

A 2.5 M solution of n-butyllithium in hexanes (4.33 mL, 10.8 mmol) was added to a stirred solution of diisopropylamine (1.1 g, 10.8 mmol) in THE (48 mL) at 0° C. The mixture was stirred at 0° C. for thirty minutes, and then cooled to −78° C. A solution of 2,3-difluoro-1-bromobenzene (1.9 g, 9.8 mmol) in THE (5 mL) was added dropwise. The mixture was stirred at −78° C. for an hour, and N-methoxy-N-methyl-5-oxo-1-((2-(trimethylsilyl)ethoxy)methyl)-pyrrolidine-2-carboxamide (2.98 g, 9.8 mmol) was added. The reaction was stirred at −78° C. for thirty minutes, and allowed to warm to 0° C., and then quenched with water. The mixture was extracted twice with ethyl acetate. The combined organic layers were washed with water, brine, dried (Na₂SO₄), and then concentrated. The residue was purified via MPLC eluting with 0-10% methanol in dichloromethane to afford 5-(4-bromo-2,3-difluorobenzoyl)-1-((2-(trimethylsilyl)ethoxy)methyl)pyrrolidin-2-one (580 mg, 14%).

### Part V—Synthesis of 5-Fluoro-N-(2-fluoro-3-(7-fluoro-3-(5-oxopyrrolidin-2-yl)-1H-indazol-6-yl)phenyl)-2-methoxypyridine-3-sulfonamide

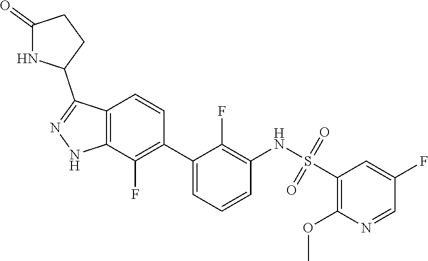 [SMILES: COc1ncc(F)cc1S(=O)(=O)Nc1cccc(-c2ccc3c(C4CCC(=O)N4)n[nH]c3c2F)c1F]

5-Fluoro-N-(2-fluoro-3-(7-fluoro-3-(5-oxopyrrolidin-2-yl)-1H-indazol-6-yl)phenyl)-2-methoxypyridine-3-sulfonamide was prepared from 5-(4-bromo-2,3-difluorobenzoyl)-1-((2-(trimethylsilyl)ethoxy)methyl)pyrrolidin-2-one using the procedures described in Part III-IX of Example 1. ¹H NMR (400 MHz, DMSO-d₆) δ 13.50-13.78 (m, 1H), 10.42 (s, 1H), 8.38 (m, 1H), 8.00 (dd, J=7.24, 2.93 Hz, 1H), 7.52 (d, J=8.41 Hz, 1H), 7.14-7.40 (m, 3H), 7.00 (br dd, J=8.02, 6.06 Hz, 1H), 5.29 (m, 1H), 4.98 (d, J=10.37 Hz, 1H), 3.72-3.83 (s, 3H), 2.28-2.45 (m, 2H), 1.94-2.19 (m, 2H). (ES, m/z): (M+H)⁺ 518.

### Example 13—Synthesis of 5-Fluoro-N-(2-fluoro-3-(7-fluoro-3-(4-(hydroxymethyl)-1H-imidazol-2-yl)-1H-indazol-6-yl)phenyl)-2-methoxypyridine-3-sulfonamide

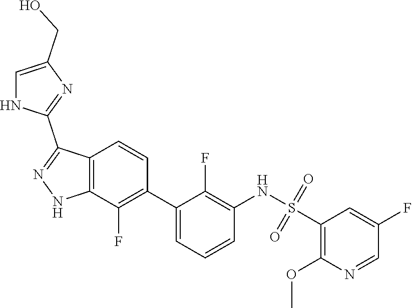 [SMILES: COc1ncc(F)cc1S(=O)(=O)Nc1cccc(-c2ccc3c(-c4nc(CO)c[nH]4)n[nH]c3c2F)c1F]

### Part I—Synthesis of 4-(((tert-Butyldimethylsilyl)oxy)methyl)-1H-imidazole

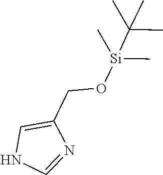

A mixture of 4-hydroxymethyl imidazole hydrochloride (3.6 g, 26.8 mmol), tert-butyldimethylchlorosilane (4.44 g, 29.4 mmol), and imidazole (4.0 g, 58.9 mmol) and DMF (27 mL) were stirred overnight at room temperature. The mixture was concentrated, and the residue was partitioned between ethyl acetate and water. The organic phase was washed with water, brine, dried (Na₂SO₄), and concentrated. The residue was purified via MPLC eluting with 20% ethyl acetate in hexanes to afford 4-(((tert-butyldimethylsilyl)oxy)methyl)-1H-imidazole (5.67 g, 100%).

### Part II—Synthesis of 4-(((tert-Butyldimethylsilyl)oxy)methyl)-1-((2-(trimethylsilyl)-ethoxy)methyl)-1H-imidazole

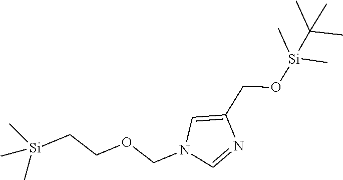 [SMILES: CC(C)(C)[Si](C)(C)OCc1cn(COCC[Si](C)(C)C)cn1]

Sodium hydride (1.13 g, 29.4 mmol) was added to a stirred solution of 4-(((tert-butyldimethylsilyl)oxy)methyl)-1H-imidazole (5.67 g, 26.7 mmol) in THE (134 mL) at 0° C. The mixture was stirred for an hour, and 2-(trimethylsilyl)ethoxymethyl chloride (4.91 g, 29.4 mmol) was added and the mixture was allowed to warm to room temperature. After four hours, the reaction was quenched with water, and extracted with ethyl acetate. The organic layer was washed with brine, dried (Na₂SO₄), and concentrated. The residue was purified via MPLC eluting with 20% ethyl acetate in hexanes to afford 4-(((tert-butyldimethylsilyl)oxy)methyl)-1-((2-(trimethylsilyl)ethoxy)methyl)-1H-imidazole (3.71 g, 40%).

### Part III—Synthesis of (4-Bromo-2,3-difluorophenyl)(4-(((tert-butyldimethylsilyl)-oxy)methyl)-1-((2-(trimethylsilyl)ethoxy)methyl)-1H-imidazol-2-yl)methanol

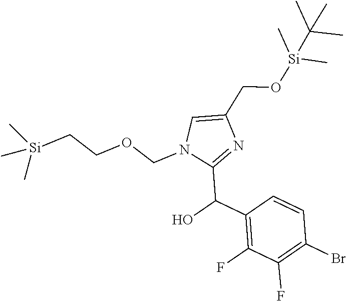 [SMILES: CC(C)(C)[Si](C)(C)OCc1cn(COCC[Si](C)(C)C)c(C(O)c2ccc(Br)c(F)c2F)n1]

A 2.5 M solution of n-butyl lithium in hexanes (4.33 mL, 10.8 mmol) was added dropwise to a stirred solution of 4-(((tert-butyldimethylsilyl)oxy)methyl)-1-((2-(trimethylsilyl)ethoxy)methyl)-1H-imidazole (3.71 g, 10.8 mmol) in THE (55 mL) at −78° C. The mixture was allowed to warm to −20° C. where it was held for thirty minutes. The solution was then re-cooled to −78° C., and 4-bromo-2,3-difluorobenzaldehyde (2.39 g, 10.8 mmol) was added. The reaction was stirred at −78° C. for two hours, and quenched with saturated aqueous ammonium chloride. The mixture was partitioned between water and ethyl acetate. The organic layer was dried (Na₂SO₄) and concentrated. The residue was purified via MPLC eluting with a gradient—of 0-50% ethyl acetate in hexanes to afford (4-bromo-2,3-difluorophenyl)(4-(((tert-butyldimethylsilyl)oxy)methyl)-1-((2-(trimethylsilyl)ethoxy)methyl)-1H-imidazol-2-yl)methanol (5.11 g, 84%).

### Part IV—Synthesis of (4-Bromo-2,3-difluorophenyl)(4-(((tert-butyldimethylsilyl)-oxy)methyl)-1-((2-(trimethylsilyl)ethoxy)methyl)-1H-imidazol-2-yl)methanone

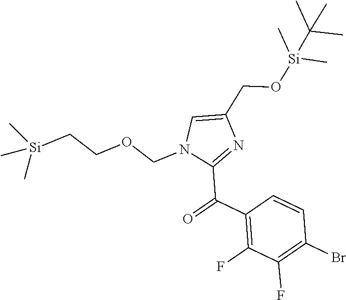 [SMILES: CC(C)(C)[Si](C)(C)OCc1cn(COCC[Si](C)(C)C)c(C(=O)c2ccc(Br)c(F)c2F)n1]

A mixture of (4-bromo-2,3-difluorophenyl)(4-(((tert-butyldimethylsilyl)oxy)-methyl)-1-((2-(trimethylsilyl)ethoxy)methyl)-1H-imidazol-2-yl)methanol (5.11 g, 9.1 mmol), dichloromethane (91 mL), and manganese dioxide (7.88 g, 91 mmol) was stirred at room temperature overnight. The mixture was filtered through a plug of celite, and the filtrate was concentrated to afford (4-bromo-2,3-difluorophenyl)(4-(((tert-butyldimethylsilyl)oxy)methyl)-1-((2-(trimethylsilyl)ethoxy)methyl)-1H-imidazol-2-yl)methanone (4.8 g, 94%) as a light yellow oil.

### Part V—Synthesis of 5-Fluoro-N-(2-fluoro-3-(7-fluoro-3-(4-(hydroxymethyl)-1H-imidazol-2-yl)-1H-indazol-6-yl)phenyl)-2-methoxypyridine-3-sulfonamide

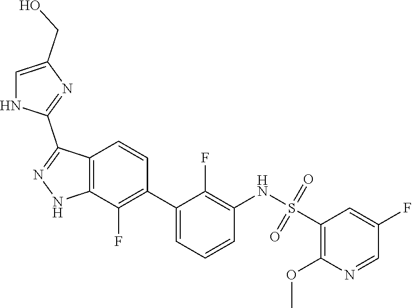 [SMILES: COc1ncc(F)cc1S(=O)(=O)Nc1cccc(-c2ccc3c(-c4nc(CO)c[nH]4)n[nH]c3c2F)c1F]

5-Fluoro-N-(2-fluoro-3-(7-fluoro-3-(4-(hydroxymethyl)-1H-imidazol-2-yl)-1H-indazol-6-yl)phenyl)-2-methoxypyridine-3-sulfonamide was prepared from (4-bromo-2,3-difluorophenyl)(4-(((tert-butyldimethylsilyl)oxy)methyl)-1-((2-(trimethylsilyl)ethoxy)methyl)-1H-imidazol-2-yl)methanone using the procedures described in Part III-IX of Example 1. ¹H NMR (400 MHz, DMSO-d₆) δ 10.45 (s, 1H), 8.44 (d, J=2.93 Hz, 1H), 8.14 (d, J=8.41 Hz, 1H), 8.01 (br dd, J=7.24, 2.93 Hz, 1H), 7.53 (br s, 1H), 7.18-7.43 (m, 4H), 4.56 (s, 2H), 3.90 (s, 3H). (ES, m/z): (M+H)⁺ 531.

### Example 14—Synthesis of N-(2,4-Difluoro-3-(7-fluoro-3-(6-methoxypyridin-2-yl)-1H-indazol-6-yl)phenyl)-5-fluoro-2-methylpyridine-3-sulfonamide

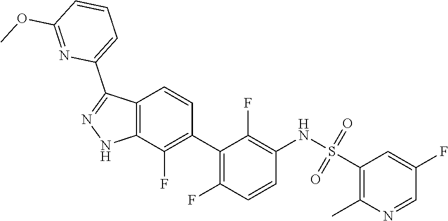 [SMILES: COc1cccc(-c2n[nH]c3c(F)c(-c4c(F)ccc(NS(=O)(=O)c5cc(F)cnc5C)c4F)ccc23)n1]

### Part I—Synthesis of 6-Bromo-7-fluoro-3-(6-methoxypyridin-2-yl)-1-((2-(trimethylsilyl)-ethoxy)methyl)-1H-indazole

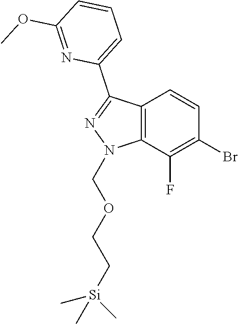 [SMILES: COc1cccc(-c2nn(COCC[Si](C)(C)C)c3c(F)c(Br)ccc23)n1]

A mixture of 6-bromo-7-fluoro-3-iodo-1-((2-(trimethylsilyl)ethoxy)methyl)-1H-indazole (500 mg, 1.06 mmol), 2-methoxy-6-(4,4,5,5-tetramethyl-1,3,2-dioxaborolan-2-yl)pyridine (262 mg, 1.11 mmol), dioxane (9 mL), potassium carbonate (440 mg, 3.18 mmol), water (3 mL), and [1,1′-bis(diphenylphosphino)ferrocene]dichloropalladium(II) (40 mg, 0.05 mmol) was heated at 90° C. overnight. The mixture was partitioned between water and ethyl acetate. The organic layer was washed with brine, dried (Na₂SO₄) and concentrated. The residue was purified via MPLC eluting with 0-50% ethyl acetate in hexanes to afford 6-bromo-7-fluoro-3-(6-methoxypyridin-2-yl)-1-((2-(trimethylsilyl)ethoxy)methyl)-1H-indazole (188 mg, 39%).

### Part II—Synthesis of N-(2,4-Difluoro-3-(7-fluoro-3-(6-methoxypyridin-2-yl)-1H-indazol-6-yl)phenyl)-5-fluoro-2-methylpyridine-3-sulfonamide

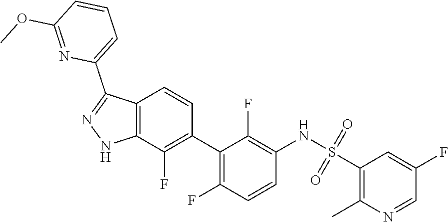 [SMILES: COc1cccc(-c2n[nH]c3c(F)c(-c4c(F)ccc(NS(=O)(=O)c5cc(F)cnc5C)c4F)ccc23)n1]

N-(2,4-Difluoro-3-(7-fluoro-3-(6-methoxypyridin-2-yl)-1H-indazol-6-yl)phenyl)-5-fluoro-2-methylpyridine-3-sulfonamide was prepared from 6-bromo-7-fluoro-3-(6-methoxypyridin-2-yl)-1-((2-(trimethylsilyl)ethoxy)methyl)-1H-indazole using the procedures described in Part IV-IX of Example 1. (ES, m/z): (M+H)⁺ 544.

### Example 15—Synthesis of N-(2,4-Difluoro-3-(7-fluoro-3-(6-oxo-1,6-dihydropyridin-2-yl)-1H-indazol-6-yl)phenyl)-5-fluoro-2-methylpyridine-3-sulfonamide

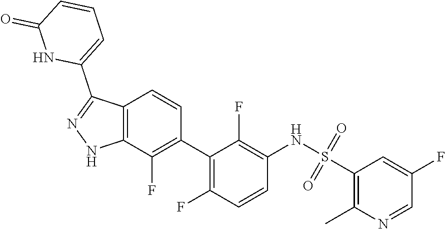 [SMILES: Cc1ncc(F)cc1S(=O)(=O)Nc1ccc(F)c(-c2ccc3c(-c4cccc(=O)[nH]4)n[nH]c3c2F)c1F]

A solution of N-(2,4-difluoro-3-(7-fluoro-3-(6-methoxypyridin-2-yl)-1H-indazol-6-yl)phenyl)-5-fluoro-2-methylpyridine-3-sulfonamide (47 mg, 0.086 mmol), ethanol (3 mL), and aqueous HBr (48%, 293 μL, 2.59 mmol) was heated at 80° C. for two hours. The reaction was cooled, and quenched with saturated aqueous sodium bicarbonate. The mixture was extracted twice with ethyl acetate. The combined organic layers were washed with brine, dried (Na₂SO₄), and concentrated. The residue was purified by preparative HPLC affording N-(2,4-difluoro-3-(7-fluoro-3-(6-oxo-1,6-dihydropyridin-2-yl)-1H-indazol-6-yl)phenyl)-5-fluoro-2-methylpyridine-3-sulfonamide (13 mg, 24%). ¹H NMR (400 MHz, DMSO-d₆) δ 10.75 (s, 1H), 10.59 (s, 1H), 8.70 (m, 1H), 7.86 (m, 1H), 7.50-7.74 (m, 2H), 7.44 (td, J=8.80, 6.06 Hz, 1H), 7.18-7.34 (m, 2H), 7.05 (br t, J=7.14 Hz, 2H), 6.55 (m, 1H), 2.73 (s, 3H). (ES, m/z): (M+H)⁺ 530.

### Example 16—Synthesis of 6-Bromo-N-(3-(dimethylamino)propyl)-7-fluoro-1-((2-(trimethylsilyl)ethoxy)methyl)-1H-indazole-3-carboxamide Intermediate

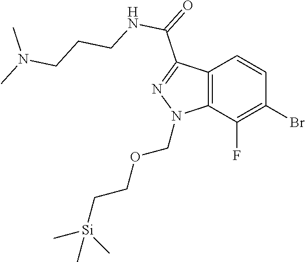 [SMILES: CN(C)CCCNC(=O)c1nn(COCC[Si](C)(C)C)c2c(F)c(Br)ccc12]

A mixture of 6-bromo-7-fluoro-3-iodo-1-((2-(trimethylsilyl)ethoxy)methyl)-1H-indazole (200 mg, 0.42 mmol), toluene (5 mL), diisopropylethylamine (110 mg, 0.85 mmol), N,N-dimethylpropane-1,3-diamine (217 mg, 2.12 mmol) and [1,1′-bis(diphenylphosphino)-ferrocene]dichloropalladium(II) (32 mg, 0.042 mmol) was stirred under an atmosphere of carbon monoxide at room temperature overnight. The mixture was concentrated, and the residue was purified via MPLC eluting with 30% methanol in dichloromethane to afford 6-bromo-N-(3-(dimethylamino)propyl)-7-fluoro-1-((2-(trimethylsilyl)ethoxy)methyl)-1H-indazole-3-carboxamide (200 mg, 100%).

### Example 17—Synthesis of N-(3-(3-(4-Chloro-1H-imidazol-2-yl)-7-fluoro-1H-indazol-6-yl)-2,4-difluorophenyl)-5-fluoro-2-methoxypyridine-3-sulfonamide

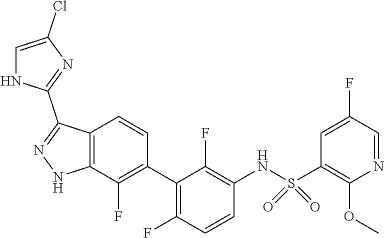 [SMILES: COc1ncc(F)cc1S(=O)(=O)Nc1ccc(F)c(-c2ccc3c(-c4nc(Cl)c[nH]4)n[nH]c3c2F)c1F]

### Part I—Synthesis of 3-(5-Chloro-1-((2-(trimethylsilyl)ethoxy)methyl)-1H-imidazol-2-yl)-6-(2,6-difluoro-3-nitrophenyl)-7-fluoro-1-((2-(trimethylsilyl)ethoxy)methyl)-1H-indazole

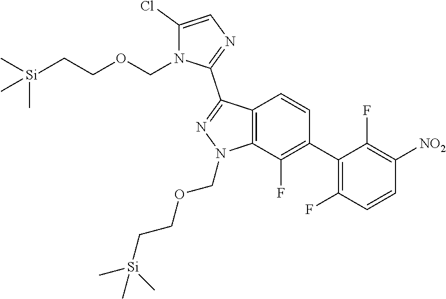 [SMILES: C[Si](C)(C)CCOCn1c(Cl)cnc1-c1nn(COCC[Si](C)(C)C)c2c(F)c(-c3c(F)ccc([N+](=O)[O-])c3F)ccc12]

A solution of 6-(2,6-difluoro-3-nitrophenyl)-7-fluoro-1-((2-(trimethylsilyl)ethoxy)-methyl)-3-(1-((2-(trimethylsilyl)ethoxy)methyl)-1H-imidazol-2-yl)-1H-indazole (210 mg, 0.34 mmol), acetonitrile (4 mL), and N-chlorosuccinimide (45 mg, 0.34 mmol) was stirred for an hour at 80° C. The solution was cooled and concentrated. The residue was purified via MPLC eluting with 25% ethyl acetate in petroleum ether to afford 3-(5-chloro-1-((2-(trimethylsilyl)-ethoxy)methyl)-1H-imidazol-2-yl)-6-(2,6-difluoro-3-nitrophenyl)-7-fluoro-1-((2-(trimethylsilyl)ethoxy)methyl)-1H-indazole (150 mg, 68%).

### Part II—Synthesis of 3-(3-(5-Chloro-1-((2-(trimethylsilyl)ethoxy)methyl)-1H-imidazol-2-yl)-7-fluoro-1-((2-(trimethylsilyl)ethoxy)methyl)-1H-indazol-6-yl)-2,4-difluoroaniline

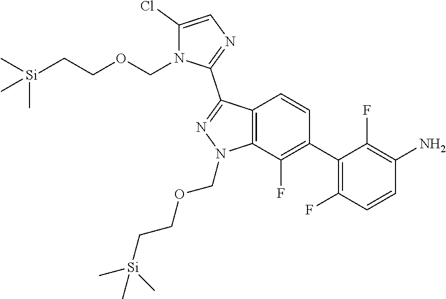 [SMILES: C[Si](C)(C)CCOCn1c(Cl)cnc1-c1nn(COCC[Si](C)(C)C)c2c(F)c(-c3c(F)ccc(N)c3F)ccc12]

A mixture of 3-(5-chloro-1-((2-(trimethylsilyl)-ethoxy)methyl)-1H-imidazol-2-yl)-6-(2,6-difluoro-3-nitrophenyl)-7-fluoro-1-((2-(trimethylsilyl)ethoxy)methyl)-1H-indazole (150 mg, 0.23 mmol), ethyl acetate (4 mL) and 10% Rh/C (150 mg, 1.46 mmol) was stirred under an atmosphere of hydrogen at room temperature overnight. The mixture was filtered and the filtrate was concentrated. The residue was purified via MPLC eluting with 25% ethyl acetate in petroleum ether to afford 3-(3-(5-chloro-1-((2-(trimethylsilyl)ethoxy)methyl)-1H-imidazol-2-yl)-7-fluoro-1-((2-(trimethylsilyl)ethoxy)methyl)-1H-indazol-6-yl)-2,4-difluoroaniline (130 mg, 91%) as a light yellow solid.

### Part III—Synthesis of N-(3-(3-(5-Chloro-1-((2-(trimethylsilyl)ethoxy)methyl)-1H-imidazol-2-yl)-7-fluoro-1-((2-(trimethylsilyl)ethoxy)methyl)-1H-indazol-6-yl)-2,4-difluorophenyl)-5-fluoro-2-methoxypyridine-3-sulfonamide

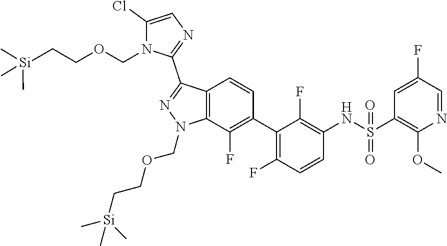

A mixture of 3-(3-(5-chloro-1-((2-(trimethylsilyl)ethoxy)methyl)-1H-imidazol-2-yl)-7-fluoro-1-((2-(trimethylsilyl)ethoxy)methyl)-1H-indazol-6-yl)-2,4-difluoroaniline (130 mg, 0.21 mmol), pyridine (5 mL), and 5-fluoro-2-methoxypyridine-3-sulfonyl chloride (390 mg, 1.73 mmol) was stirred for an hour at room temperature. The mixture was diluted with ethyl acetate and washed with 0.5 M HCl. The organic layer was dried (Na₂SO₄) and concentrated. The residue was purified via MPLC eluting with 25% ethyl acetate in petroleum ether to afford N-(3-(3-(5-chloro-1-((2-(trimethylsilyl)ethoxy)methyl)-1H-imidazol-2-yl)-7-fluoro-1-((2-(trimethylsilyl)ethoxy)methyl)-1H-indazol-6-yl)-2,4-difluorophenyl)-5-fluoro-2-methoxypyridine-3-sulfonamide (100 mg, 59%).

### Part IV—Synthesis of N-(3-(3-(4-Chloro-1H-imidazol-2-yl)-7-fluoro-1H-indazol-6-yl)-2,4-difluorophenyl)-5-fluoro-2-methoxypyridine-3-sulfonamide

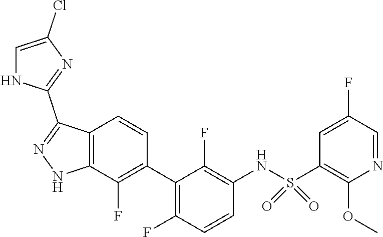 [SMILES: COc1ncc(F)cc1S(=O)(=O)Nc1ccc(F)c(-c2ccc3c(-c4nc(Cl)c[nH]4)n[nH]c3c2F)c1F]

A mixture of N-(3-(3-(5-chloro-1-((2-(trimethylsilyl)ethoxy)methyl)-1H-imidazol-2-yl)-7-fluoro-1-((2-(trimethylsilyl)ethoxy)methyl)-1H-indazol-6-yl)-2,4-difluorophenyl)-5-fluoro-2-methoxypyridine-3-sulfonamide (100 mg), dichloromethane (2 mL), and trifluoroacetic acid (1 mL) was stirred for two hours at room temperature and concentrated. The residue was stirred in 7M ammonia in methanol (1 mL) for an hour at room temperature and concentrated. The residue was purified by reverse phase HPLC eluting with a gradient of 35% to 65% acetonitrile in 10M aqueous ammonium bicarbonate to afford N-(3-(3-(4-chloro-1H-imidazol-2-yl)-7-fluoro-1H-indazol-6-yl)-2,4-difluorophenyl)-5-fluoro-2-methoxypyridine-3-sulfonamide (19.8 mg, 29%) as an off-white solid. ¹H NMR (400 MHz, CD₃OD-d₄) δ 8.26 (d, J=3.0 Hz, 1H), 8.14 (d, J=8.4 Hz, 1H), 7.95 (dd, J=7.3, 3.1 Hz, 1H), 7.57 (td, J=8.9, 5.6 Hz, 1H), 7.19 (s, 1H), 7.11 (ddd, J=11.5, 7.9, 3.5 Hz, 2H), 3.99 (s, 3H). (ES, m/z): (M+H)⁺ 553.

### Example 18—Synthesis of N-(3-(7-Chloro-3-(1H-imidazol-2-yl)-1H-indazol-6-yl)-2-fluorophenyl)-5-fluoro-2-methoxypyridine-3-sulfonamide

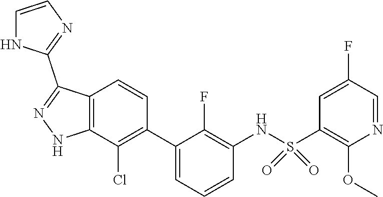 [SMILES: COc1ncc(F)cc1S(=O)(=O)Nc1cccc(-c2ccc3c(-c4ncc[nH]4)n[nH]c3c2Cl)c1F]

### Part I—Synthesis of (4-Bromo-3-chloro-2-fluorophenyl)(1-((2-(trimethylsilyl)ethoxy)-methyl)-1H-imidazol-2-yl)methanone

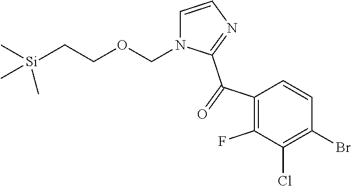 [SMILES: C[Si](C)(C)CCOCn1ccnc1C(=O)c1ccc(Br)c(Cl)c1F]

A 2M solution of isopropyl magnesium chloride lithium chloride (7.5 mL, 15 mmol) was added dropwise to a stirred solution of 1-bromo-2-chloro-3-fluoro-4-iodobenzene (2.48 g, 7.0 mmol) in THE (50 mL) at −78° C. A solution of ethyl 1-((2-(trimethylsilyl)ethoxy)methyl)-1H-imidazole-2-carboxylate (1.00 g, 3.70 mmol) in THE (2.0 mL) was added dropwise with stirring at −78° C. The resulting solution was stirred for an additional 30 minutes at −78° C., and an additional four hours at room temperature. The reaction was quenched by the addition of saturated ammonium chloride. The mixture was extracted three times with ethyl acetate. The combined organic layers were washed with brine, dried (Na₂SO₄) and concentrated. The residue was purified via MPLC eluting with a gradient of 0-25% ethyl acetate in petroleum ether to afford (4-bromo-3-chloro-2-fluorophenyl)(1-((2-(trimethylsilyl)ethoxy)-methyl)-1H-imidazol-2-yl)methanone (900 mg, 56%) as a yellow solid.

### Part II—Synthesis of N-(3-(7-Chloro-3-(1H-imidazol-2-yl)-1H-indazol-6-yl)-2-fluorophenyl)-5-fluoro-2-methoxypyridine-3-sulfonamide

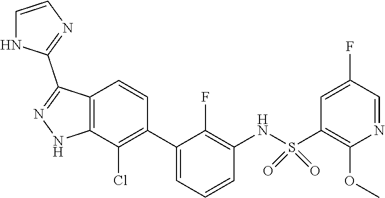 [SMILES: COc1ncc(F)cc1S(=O)(=O)Nc1cccc(-c2ccc3c(-c4ncc[nH]4)n[nH]c3c2Cl)c1F]

N-(3-(7-Chloro-3-(1H-imidazol-2-yl)-1H-indazol-6-yl)-2-fluorophenyl)-5-fluoro-2-methoxypyridine-3-sulfonamide was prepared from (4-bromo-3-chloro-2-fluorophenyl)(1-((2-(trimethylsilyl)ethoxy)-methyl)-1H-imidazol-2-yl)methanone using the procedures described in Part III-IX of Example 1. ¹H NMR (400 MHz, CD₃OD-d₄) δ 8.27 (s, 1H), 8.15 (d, J=8.4 Hz, 1H), 7.96 (dd, J=7.2, 3.0 Hz, 1H), 7.74 (s, 2H), 7.62 (m, 1H), 7.17-7.37 (m, 3H), 4.02 (s, 3H). (ES, m/z): (M+H)⁺ 517.

### Example 19—Synthesis of N-(3-(3-(4-Cyano-1H-imidazol-2-yl)-7-fluoro-1H-indazol-6-yl)-2,4-difluorophenyl)-5-fluoro-2-methoxypyridine-3-sulfonamide

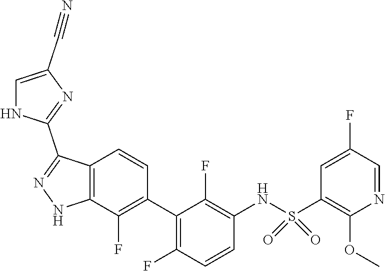 [SMILES: COc1ncc(F)cc1S(=O)(=O)Nc1ccc(F)c(-c2ccc3c(-c4nc(C#N)c[nH]4)n[nH]c3c2F)c1F]

### Part I—Synthesis of 3-(5-Bromo-1-((2-(trimethylsilyl)ethoxy)methyl)-1H-imidazol-2-yl)-6-(2,6-difluoro-3-nitrophenyl)-7-fluoro-1-((2-(trimethylsilyl)ethoxy)methyl)-1H-indazole

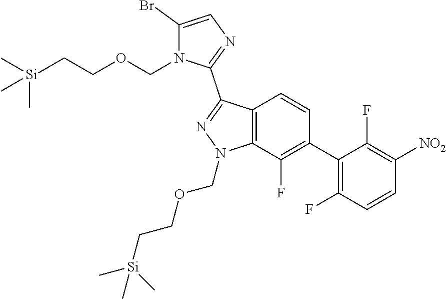 [SMILES: C[Si](C)(C)CCOCn1c(Br)cnc1-c1nn(COCC[Si](C)(C)C)c2c(F)c(-c3c(F)ccc([N+](=O)[O-])c3F)ccc12]

A solution of 6-(2,6-difluoro-3-nitrophenyl)-7-fluoro-1-((2-(trimethylsilyl)ethoxy)-methyl)-3-(1-((2-(trimethylsilyl)ethoxy)methyl)-1H-imidazol-2-yl)-1H-indazole (300 mg, 0.484 mmol), acetonitrile (5 mL), and N-bromosuccinimide (86 mg, 0.484 mmol) was stirred for an hour at 80° C. The solution was cooled and partitioned between water and ethyl acetate. The organic layer was dried (Na₂SO₄) and concentrated. The residue was purified via MPLC eluting with 25% ethyl acetate in petroleum ether to afford 3-(5-bromo-1-((2-(trimethylsilyl)-ethoxy)methyl)-1H-imidazol-2-yl)-6-(2,6-difluoro-3-nitrophenyl)-7-fluoro-1-((2-(trimethylsilyl)ethoxy)methyl)-1H-indazole (250 mg, 74%).

### Part II—Synthesis of 2-(6-(2,6-Difluoro-3-nitrophenyl)-7-fluoro-1-((2-(trimethylsilyl)-ethoxy)methyl)-1H-indazol-3-yl)-1-((2-(trimethylsilyl)ethoxy)methyl)-1H-imidazole-5-carbonitrile

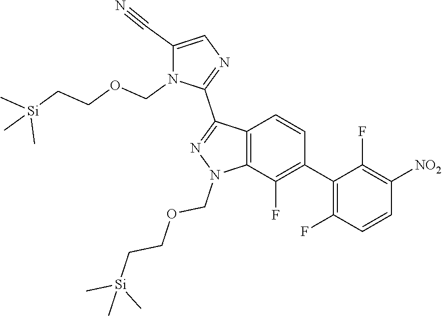

A mixture of 3-(5-bromo-1-((2-(trimethylsilyl)-ethoxy)methyl)-1H-imidazol-2-yl)-6-(2,6-difluoro-3-nitrophenyl)-7-fluoro-1-((2-(trimethylsilyl)ethoxy)methyl)-1H-indazole (300 mg, 0.43 mmol), DMF (3 mL), zinc cyanide (151 mg, 1.29 mmol), and tetrakis(triphenylphosphine)palladium(0) (99 mg, 0.086 mmol) was stirred for an hour at 180° C. The mixture was cooled, and partitioned between ethyl acetate and water. The organic layer was dried (Na₂SO₄) and concentrated. The residue was purified via MPLC eluting with 25% ethyl acetate in petroleum ether to afford 2-(6-(2,6-difluoro-3-nitrophenyl)-7-fluoro-1-((2-(trimethylsilyl)-ethoxy)methyl)-1H-indazol-3-yl)-1-((2-(trimethylsilyl)ethoxy)methyl)-1H-imidazole-5-carbonitrile (200 mg, 72%).

### Part III—Synthesis of 2-(6-(3-Amino-2,6-difluorophenyl)-7-fluoro-1-((2-(trimethylsilyl)ethoxy)methyl)-1H-indazol-3-yl)-1-((2-(trimethylsilyl)ethoxy)methyl)-1H-imidazole-5-carbonitrile

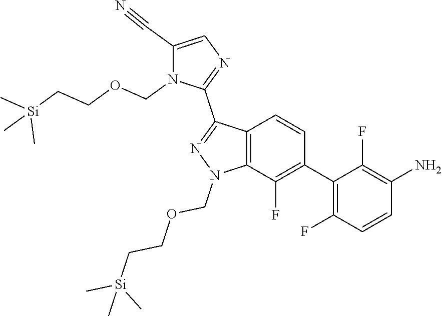

A mixture of 2-(6-(2,6-difluoro-3-nitrophenyl)-7-fluoro-1-((2-(trimethylsilyl)-ethoxy)methyl)-1H-indazol-3-yl)-1-((2-(trimethylsilyl)ethoxy)methyl)-1H-imidazole-5-carbonitrile (360 mg, 0.56 mmol), ethanol (5 mL), iron powder (156 mg, 2.79 mmol), and ammonium chloride (150 mg, 2.79 mmol) was stirred for two hours at 80° C. The mixture was filtered. The filtrate was concentrated, and the residue was purified via MPLC eluting with 25% ethyl acetate in petroleum ether to afford 2-(6-(3-amino-2,6-difluorophenyl)-7-fluoro-1-((2-(trimethylsilyl)ethoxy)methyl)-1H-indazol-3-yl)-1-((2-(trimethylsilyl)ethoxy)methyl)-1H-imidazole-5-carbonitrile (300 mg, 63%).

### Part IV—Synthesis of N-(3-(3-(5-Cyano-1-((2-(trimethylsilyl)ethoxy)methyl)-1H-imidazol-2-yl)-7-fluoro-1-((2-(trimethylsilyl)ethoxy)methyl)-1H-indazol-6-yl)-2,4-difluorophenyl)-5-fluoro-2-methoxypyridine-3-sulfonamide

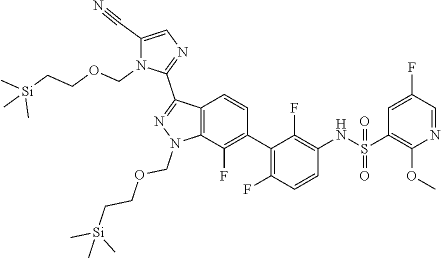

A mixture of 2-(6-(3-amino-2,6-difluorophenyl)-7-fluoro-1-((2-(trimethylsilyl)ethoxy)methyl)-1H-indazol-3-yl)-1-((2-(trimethylsilyl)ethoxy)methyl)-1H-imidazole-5-carbonitrile (300 mg, 0.49 mmol), pyridine (10 mL), and 5-fluoro-2-methoxypyridine-3-sulfonyl chloride (330 mg, 1.46 mmol) was stirred overnight at room temperature. The mixture was diluted with ethyl acetate and washed with 0.5 M HCl. The organic layer was dried (Na₂SO₄) and concentrated. The residue was purified via MPLC eluting with 25% ethyl acetate in petroleum ether to afford N-(3-(3-(5-cyano-1-((2-(trimethylsilyl)ethoxy)methyl)-1H-imidazol-2-yl)-7-fluoro-1-((2-(trimethylsilyl)ethoxy)methyl)-1H-indazol-6-yl)-2,4-difluorophenyl)-5-fluoro-2-methoxypyridine-3-sulfonamide (100 mg, 25%).

### Part V—Synthesis of N-(3-(3-(4-Cyano-1H-imidazol-2-yl)-7-fluoro-1H-indazol-6-yl)-2,4-difluorophenyl)-5-fluoro-2-methoxypyridine-3-sulfonamide

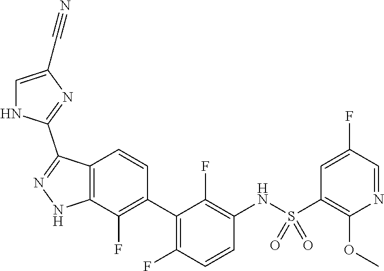

A mixture of N-(3-(3-(5-cyano-1-((2-(trimethylsilyl)ethoxy)methyl)-1H-imidazol-2-yl)-7-fluoro-1-((2-(trimethylsilyl)ethoxy)methyl)-1H-indazol-6-yl)-2,4-difluorophenyl)-5-fluoro-2-methoxypyridine-3-sulfonamide (100 mg), dichloromethane (2 mL), and trifluoroacetic acid (1 mL) was stirred for two hours at room temperature and concentrated. The residue was stirred in 7M ammonia in methanol (1 mL) for an hour at room temperature and concentrated. The residue was purified by reverse phase HPLC eluting with a gradient of 35% to 50% acetonitrile in 10M aqueous ammonium bicarbonate to afford N-(3-(3-(4-cyano-1H-imidazol-2-yl)-7-fluoro-1H-indazol-6-yl)-2,4-difluorophenyl)-5-fluoro-2-methoxypyridine-3-sulfonamide (6.4 mg, 15%) as an off-white solid. ¹H NMR (400 MHz, CD₃OD-d₄) δ 8.28 (d, J=3.0 Hz, 1H), 8.20 (d, J=8.4 Hz, 1H), 7.99 (s, 1H), 7.95 (dd, J=7.2, 3.0 Hz, 1H), 7.59 (td, J=8.9, 5.7 Hz, 1H), 7.17-7.07 (m, 2H), 4.00 (s, 3H). (ES, m/z): (M+H)⁺ 544.

### Example 20—Synthesis of 2-(6-(2,6-Difluoro-3-((5-fluoro-2-methoxypyridine)-3-sulfonamido)phenyl)-7-fluoro-1H-indazol-3-yl)-1H-imidazole-4-carboxamide

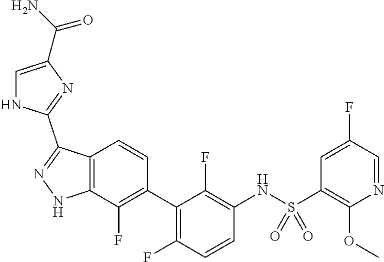

### Part I—Synthesis of 2-(6-(2,6-Difluoro-3-((5-fluoro-2-methoxypyridine)-3-sulfonamido)phenyl)-7-fluoro-1-((2-(trimethylsilyl)ethoxy)methyl)-1H-indazol-3-yl)-1-((2-(trimethylsilyl)ethoxy)methyl)-1H-imidazole-5-carboxamide

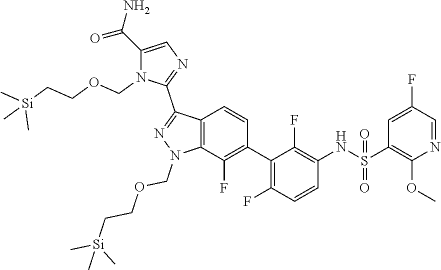

A solution of N-(3-(3-(5-cyano-1-((2-(trimethylsilyl)ethoxy)methyl)-1H-imidazol-2-yl)-7-fluoro-1-((2-(trimethylsilyl)ethoxy)methyl)-1H-indazol-6-yl)-2,4-difluorophenyl)-5-fluoro-2-methoxypyridine-3-sulfonamide (70 mg, 0.087 mmol), DMSO (2 mL), potassium carbonate (36 mg, 0.26 mmol), and 30% hydrogen peroxide in water (600 μL) was stirred for two hours at room temperature. The mixture was concentrated to afford 2-(6-(2,6-difluoro-3-((5-fluoro-2-methoxypyridine)-3-sulfonamido)phenyl)-7-fluoro-1-((2-(trimethylsilyl)ethoxy)methyl)-1H-indazol-3-yl)-1-((2-(trimethylsilyl)ethoxy)methyl)-1H-imidazole-5-carboxamide (60 mg, 84%) as a light yellow solid.

### Part II—Synthesis of 2-(6-(2,6-Difluoro-3-((5-fluoro-2-methoxypyridine)-3-sulfonamido)phenyl)-7-fluoro-1H-indazol-3-yl)-1H-imidazole-4-carboxamide

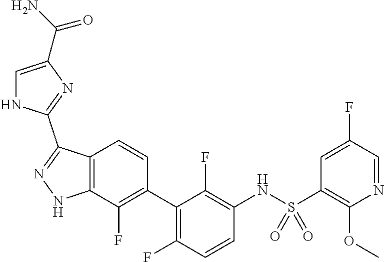

A mixture of 2-(6-(2,6-difluoro-3-((5-fluoro-2-methoxypyridine)-3-sulfonamido)phenyl)-7-fluoro-1-((2-(trimethylsilyl)ethoxy)methyl)-1H-indazol-3-yl)-1-((2-(trimethylsilyl)ethoxy)methyl)-1H-imidazole-5-carboxamide (60 mg), dichloromethane (2 mL), and trifluoroacetic acid (1 mL) was stirred for two hours at room temperature and concentrated. The residue was stirred in 7M ammonia in methanol (1 mL) for an hour at room temperature and concentrated. The residue was purified by reverse phase HPLC eluting with a gradient of 25% to 55% acetonitrile in 10M aqueous ammonium bicarbonate to afford 2-(6-(2,6-difluoro-3-((5-fluoro-2-methoxypyridine)-3-sulfonamido)phenyl)-7-fluoro-1H-indazol-3-yl)-1H-imidazole-4-carboxamide (8.9 mg, 22%) as an off-white solid. ¹H NMR (400 MHz, CD₃OD-d₄) δ 8.36 (d, J=8.4 Hz, 1H), 8.28 (d, J=3.0 Hz, 1H), 7.95 (dd, J=7.3, 3.0 Hz, 1H), 7.81 (s, 1H), 7.58 (td, J=8.7, 5.6 Hz, 1H), 7.11 (q, J=7.0, 5.3 Hz, 2H), 4.00 (s, 3H). (ES, m/z): (M+H)⁺ 562.

### Example 21—Synthesis of N-(3-(3-(4-(1,2-Dihydroxyethyl)-1H-imidazol-2-yl)-7-fluoro-1H-indazol-6-yl)-2,4-difluorophenyl)-5-fluoro-2-methoxypyridine-3-sulfonamide

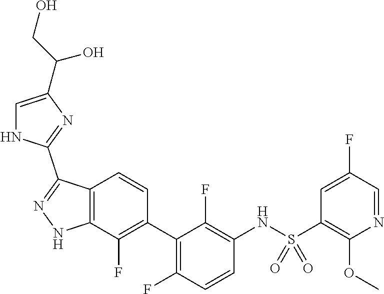

### Part I—Synthesis of N-(3-(3-(5-Bromo-1-((2-(trimethylsilyl)ethoxy)methyl)-1H-imidazol-2-yl)-7-fluoro-1-((2-(trimethylsilyl)ethoxy)methyl)-1H-indazol-6-yl)-2,4-difluorophenyl)-5-fluoro-2-methoxypyridine-3-sulfonamide

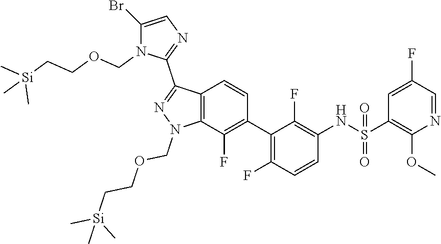

A solution of N-(2,4-difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)phenyl)-5-fluoro-2-methoxypyridine-3-sulfonamide (1.00 g, 1.28 mmol), N-bromosuccinimide (228 mg, 1.28 mmol) and acetonitrile (20 mL) was stirred for three hours at 60° C. The mixture was cooled, then partitioned between ethyl acetate and water. The organic layer was dried (Na₂SO₄) and concentrated. The residue was purified via MPLC eluting with 16% ethyl acetate in petroleum ether to afford N-(3-(3-(5-bromo-1-((2-(trimethylsilyl)ethoxy)methyl)-1H-imidazol-2-yl)-7-fluoro-1-((2-(trimethylsilyl)ethoxy)methyl)-1H-indazol-6-yl)-2,4-difluorophenyl)-5-fluoro-2-methoxypyridine-3-sulfonamide (500 mg, 45%).

### Part II—Synthesis of N-(2,4-Difluoro-3-(7-fluoro-1-((2-(trimethylsilyl)ethoxy)methyl)-3-(1-((2-(trimethylsilyl)ethoxy)methyl)-5-vinyl-1H-imidazol-2-yl)-1H-indazol-6-yl)phenyl)-5-fluoro-2-methoxypyridine-3-sulfonamide

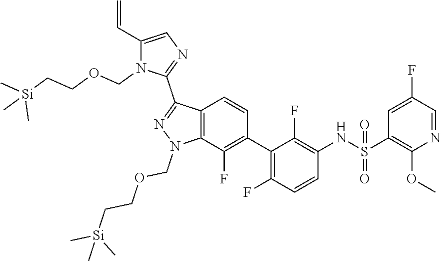

A mixture of N-(3-(3-(5-bromo-1-((2-(trimethylsilyl)ethoxy)methyl)-1H-imidazol-2-yl)-7-fluoro-1-((2-(trimethylsilyl)ethoxy)methyl)-1H-indazol-6-yl)-2,4-difluorophenyl)-5-fluoro-2-methoxypyridine-3-sulfonamide (500 mg, 0.58 mmol), 2-ethenyl-4,4,5,5-tetramethyl-1,3,2-dioxaborolane (108 mg, 0.70 mmol), tetrakis(triphenylphosphine)-palladium(0) (67 mg, 0.058 mmol), potassium carbonate (242 mg, 1.75 mmol), toluene (15 mL), ethanol (5 mL), and water (5 mL) was stirred overnight at 100° C. The mixture was cooled, then partitioned between ethyl acetate and water. The organic layer was dried (Na₂SO₄) and concentrated. The residue was purified via MPLC eluting with 25% ethyl acetate in petroleum ether to afford N-(2,4-difluoro-3-(7-fluoro-1-((2-(trimethylsilyl)ethoxy)methyl)-3-(1-((2-(trimethylsilyl)ethoxy)methyl)-5-vinyl-1H-imidazol-2-yl)-1H-indazol-6-yl)phenyl)-5-fluoro-2-methoxypyridine-3-sulfonamide (350 mg, 75%).

### Part III—Synthesis of N-(3-(3-(5-(1,2-Dihydroxyethyl)-1-((2-(trimethylsilyl)ethoxy) methyl)-1H-imidazol-2-yl)-7-fluoro-1-((2-(trimethylsilyl)ethoxy)methyl)-1H-indazol-6-yl)-2,4-difluorophenyl)-5-fluoro-2-methoxypyridine-3-sulfonamide

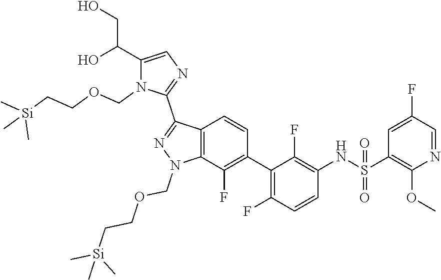

A mixture of N-(2,4-difluoro-3-(7-fluoro-1-((2-(trimethylsilyl)ethoxy)methyl)-3-(1-((2-(trimethylsilyl)ethoxy)methyl)-5-vinyl-1H-imidazol-2-yl)-1H-indazol-6-yl)phenyl)-5-fluoro-2-methoxypyridine-3-sulfonamide (80 mg, 0.099 mmol), dioxane (5 mL), water (5 mL), sodium hydroxide (20 mg, 0.50 mmol), and potassium permanganate (79 mg, 0.50 mmol) was stirred overnight at room temperature. The mixture was filtered, and the filtrate was extracted three times with ethyl acetate. The combined organic layers were dried (Na₂SO₄) and concentrated. The residue was purified via MPLC eluting with 50% ethyl acetate in petroleum ether to afford N-(3-(3-(5-(1,2-dihydroxyethyl)-1-((2-(trimethylsilyl)ethoxy)methyl)-1H-imidazol-2-yl)-7-fluoro-1-((2-(trimethylsilyl)ethoxy)methyl)-1H-indazol-6-yl)-2,4-difluorophenyl)-5-fluoro-2-methoxypyridine-3-sulfonamide (50 mg, 60%) as a yellow solid.

### Part IV—Synthesis of N-(3-(3-(4-(1,2-Dihydroxyethyl)-1H-imidazol-2-yl)-7-fluoro-1H-indazol-6-yl)-2,4-difluorophenyl)-5-fluoro-2-methoxypyridine-3-sulfonamide

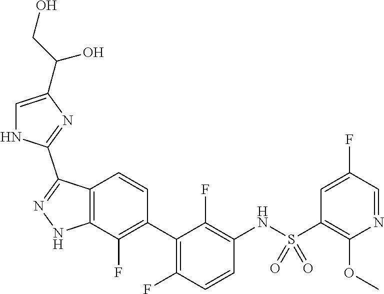

A mixture of N-(3-(3-(5-(1,2-dihydroxyethyl)-1-((2-(trimethylsilyl)-ethoxy)methyl)-1H-imidazol-2-yl)-7-fluoro-1-((2-(trimethylsilyl)ethoxy)methyl)-1H-indazol-6-yl)-2,4-difluorophenyl)-5-fluoro-2-methoxypyridine-3-sulfonamide (50 mg, 0.06 mmol), dichloromethane (2 mL), and trifluoroacetic acid (2 mL) was stirred for two hours at room temperature and concentrated. The residue was purified by reverse phase HPLC eluting with a gradient of 30% to 50% acetonitrile in 10M aqueous ammonium bicarbonate to afford N-(3-(3-(4-(1,2-dihydroxyethyl)-1H-imidazol-2-yl)-7-fluoro-1H-indazol-6-yl)-2,4-difluorophenyl)-5-fluoro-2-methoxypyridine-3-sulfonamide (5.5 mg, 16%) as a white solid. ¹H NMR (400 MHz, CD₃OD-d₄) δ 8.27 (d, J=2.8 Hz, 1H), 8.16 (d, J=8.4 Hz, 1H), 7.97 (m, 1H), 7.58 (m, 1H), 7.19 (s, H), 7.13-7.05 (m, 2H), 4.00 (s, 3H), 3.91 (m, 1H), 3.83 (m, 1H). (ES, m/z): (M+H)⁺ 579.

### Example 22—Synthesis of N-(3-(3-(1H-Imidazol-2-yl)-7-methyl-1H-indazol-6-yl)-2-fluorophenyl)-5-fluoro-2-methoxypyridine-3-sulfonamide

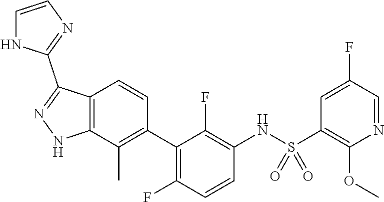

### Part I—Synthesis of 6-(2-Fluoro-3-nitrophenyl)-7-methyl-1-((2-(trimethylsilyl)ethoxy)-methyl)-3-(1-((2-(trimethylsilyl)ethoxy)methyl)-1H-imidazol-2-yl)-1H-indazole

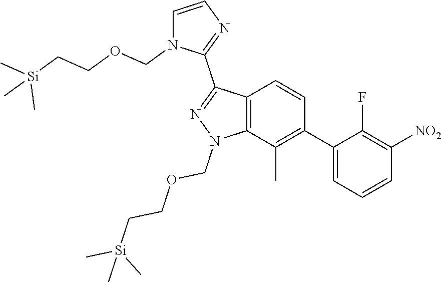

A mixture of 7-chloro-6-(2-fluoro-3-nitrophenyl)-1-((2-(trimethylsilyl)ethoxy)methyl)-3-(1-((2-(trimethylsilyl)ethoxy)methyl)-1H-imidazol-2-yl)-1H-indazole (240 mg, 0.388 mmol), trimethyl-1,3,5,2,4,6-trioxatriborinane (487 mg, 3.88 mmol), dioxane (10 mL), potassium phosphate (165 mg, 0.776 mmol), water (2 mL), tricyclohexylphosphine (10.9 mg, 0.039 mmol), and [(tricyclohexylphosphine)-2-(2′-aminobiphenyl)]palladium(II) methanesulfonate (33 mg, 0.039 mmol) was stirred overnight at 80° C. and concentrated. The residue was purified via MPLC eluting with 25% ethyl acetate in petroleum ether to afford 6-(2-fluoro-3-nitrophenyl)-7-methyl-1-((2-(trimethylsilyl)ethoxy)-methyl)-3-(1-((2-(trimethylsilyl)ethoxy)methyl)-1H-imidazol-2-yl)-1H-indazole (230 mg, 99%).

### Part II—Synthesis of 2-Fluoro-3-(7-methyl-1-((2-(trimethylsilyl)ethoxy)methyl)-3-(1-((2-(trimethylsilyl)ethoxy)methyl)-1H-imidazol-2-yl)-1H-indazol-6-yl)aniline

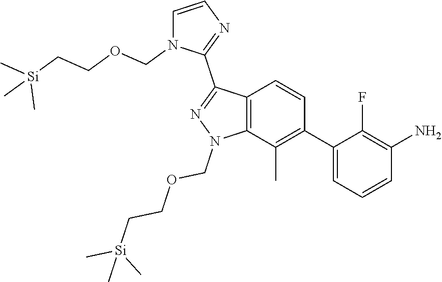

A mixture of 6-(2-fluoro-3-nitrophenyl)-7-methyl-1-((2-(trimethylsilyl)ethoxy)-methyl)-3-(1-((2-(trimethylsilyl)ethoxy)methyl)-1H-imidazol-2-yl)-1H-indazole (331 mg, 0.554 mmol), ethyl acetate (20 mL), and 10% palladium on carbon (330 mg, 3.101 mmol) was stirred under an atmosphere of hydrogen for three hours at room temperature. The mixture was filtered through celite. The filtrate was concentrated to afford 2-fluoro-3-(7-methyl-1-((2-(trimethylsilyl)ethoxy)methyl)-3-(1-((2-(trimethylsilyl)ethoxy)methyl)-1H-imidazol-2-yl)-1H-indazol-6-yl)aniline (300 mg) which was used without further purification.

### Part III—Synthesis of 5-Fluoro-N-(2-fluoro-3-(7-methyl-1-((2-(trimethylsilyl)ethoxy)methyl)-3-(1-((2-(trimethylsilyl)ethoxy)methyl)-1H-imidazol-2-yl)-1H-indazol-6-yl)phenyl)-2-methoxypyridine-3-sulfonamide

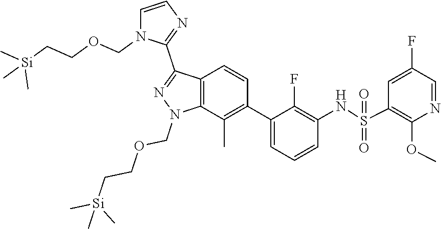

A mixture of 2-fluoro-3-(7-methyl-1-((2-(trimethylsilyl)ethoxy)methyl)-3-(1-((2-(trimethylsilyl)ethoxy)methyl)-1H-imidazol-2-yl)-1H-indazol-6-yl)aniline (150 mg, 0.264 mmol), pyridine (10 mL), and 5-fluoro-2-methoxypyridine-3-sulfonyl chloride (200 mg, 0.975 mmol) was stirred overnight at room temperature and concentrated. The residue was purified via MPLC eluting with 25% ethyl acetate in petroleum ether to afford 5-fluoro-N-(2-fluoro-3-(7-methyl-1-((2-(trimethylsilyl)ethoxy)methyl)-3-(1-((2-(trimethylsilyl)ethoxy)methyl)-1H-imidazol-2-yl)-1H-indazol-6-yl)phenyl)-2-methoxypyridine-3-sulfonamide (80 mg, 40%).

### Part IV—Synthesis of N-(3-(3-(1H-Imidazol-2-yl)-7-methyl-1H-indazol-6-yl)-2-fluorophenyl)-5-fluoro-2-methoxypyridine-3-sulfonamide

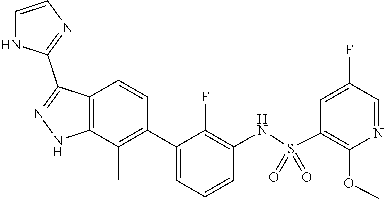 [SMILES: COc1ncc(F)cc1S(=O)(=O)Nc1cccc(-c2ccc3c(-c4ncc[nH]4)n[nH]c3c2C)c1F]

A mixture of 5-fluoro-N-(2-fluoro-3-(7-methyl-1-((2-(trimethylsilyl)ethoxy)methyl)-3-(1-((2-(trimethylsilyl)ethoxy)methyl)-1H-imidazol-2-yl)-1H-indazol-6-yl)phenyl)-2-methoxypyridine-3-sulfonamide (80 mg, 0.106 mmol), dichloromethane (5 mL), and trifluoroacetic acid (5 mL) was stirred for two hours at room temperature and concentrated. The residue was stirred in 7M ammonia in methanol (1 mL) for an hour at room temperature and concentrated. The residue was purified by reverse phase HPLC eluting with a gradient of 30% to 60% acetonitrile in 10M aqueous ammonium bicarbonate to afford N-(3-(3-(1H-imidazol-2-yl)-7-methyl-1H-indazol-6-yl)-2-fluorophenyl)-5-fluoro-2-methoxypyridine-3-sulfonamide (23 mg, 44%) as an off-white solid. ¹H NMR (400 MHz, CD₃OD-d₄) δ 8.26 (d, J=3.0 Hz, 1H), 8.13 (m, 1H), 7.95 (dd, J=7.3, 3.0 Hz, 1H), 7.52 (td, J=7.8, 1.7 Hz, 1H), 7.16-7.30 (m, 3H), 7.13 (t, J=7.3 Hz, 1H), 7.02 (d, J=8.4 Hz, 1H), 4.02 (s, 3H), 2.19 (d, J=1.5 Hz, 3H). (ES, m/z): (M+H)⁺ 497.

### Example 23—Synthesis of N-(2-Acetamidoethyl)-6-(2,6-difluoro-3-((5-fluoro-2-methoxypyridine)-3-sulfonamido)phenyl)-7-fluoro-1H-indazole-3-carboxamide

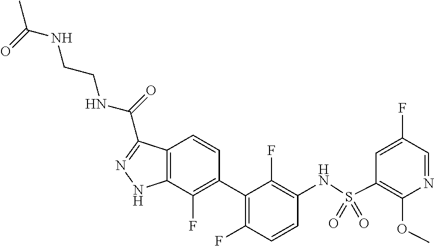 [SMILES: COc1ncc(F)cc1S(=O)(=O)Nc1ccc(F)c(-c2ccc3c(C(=O)NCCNC(C)=O)n[nH]c3c2F)c1F]

### Part I—Synthesis of 4-Bromo-2,3-difluorobenzaldehyde

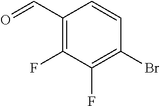 [SMILES: O=Cc1ccc(Br)c(F)c1F]

To a stirred solution of diisopropyl amine (24.23 g, 239.4 mmol) in THE (1 L) in an ice bath at 0° C. was added a solution of n-butyl lithium in hexanes (22.8 mL, 239 mmol). The mixture was stirred for an additional 30 minutes and then cooled to −78° C. A solution of 2,3-difluoro-1-bromobenzene (42.0 g, 218 mmol) in THE (50 mL) was then added dropwise. The mixture was stirred an hour at −78° C. and then DMF (84.4 mL, 1.08 mol) was added. The mixture was stirred for 30 minutes at −78° C., and then allowed to warm to 0° C. The mixture was quenched with water, and extracted twice with ethyl acetate. The organic layer was washed with brine, dried (Na₂SO₄) and concentrated. The residue was purified with flash chromatography eluting with a gradient of 5-20% ethyl acetate in hexanes to afford 4-bromo-2,3-difluorobenzaldehyde (38 g, 79%) as a yellow solid.

### Part II—Synthesis of 6-Bromo-7-fluoro-1H-indazole

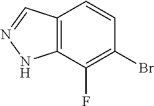 [SMILES: Fc1c(Br)ccc2cn[nH]c12]

A mixture of 4-bromo-2,3-difluorobenzaldehyde (20.0 g, 90.4 mmol), potassium carbonate (15.0 g, 106 mmol), and methoxyamine hydrochloride (8.4 g, 99.5 mmol) in dimethoxyethane (250 mL) was stirred at 40° C. for three hours. The mixture was cooled and filtered through celite, and the celite pad was washed with ethyl acetate. The combined filtrate was concentrated, and the residue was diluted with dimethoxyethane (250 mL). Hydrazine hydrate (45.4 g, 90.4 mmol) was added and the mixture was stirred at 90° C. overnight. The mixture was cooled and concentrated. The residue was triturated in water, and filtered to afford 6-bromo-7-fluoro-1H-indazole (20 g, 102%) as a light brown solid.

### Part III—Synthesis of 6-Bromo-7-fluoro-3-iodo-1H-indazole

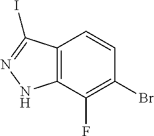 [SMILES: Fc1c(Br)ccc2c(I)n[nH]c12]

A mixture of 6-bromo-7-fluoro-1H-indazole (20 g, 93 mmol), DMF (180 mL), iodine (26 g, 102 mmol), and potassium hydroxide (20.8 g, 372 mmol) was stirred at room temperature overnight. The mixture was diluted with water, and adjusted to pH 7 with concentrated HCl. After extracting twice with ethyl acetate, the organic layer was washed with brine, and concentrated under vacuum. The residue was purified by MPLC eluting with a gradient of ethyl acetate in hexanes to afford 6-bromo-7-fluoro-3-iodo-1H-indazole (30.2 g, 95%) as a white solid.

### Part IV—Synthesis of 6-Bromo-7-fluoro-3-iodo-1-((2-(trimethylsilyl)ethoxy)methyl)-1H-indazole

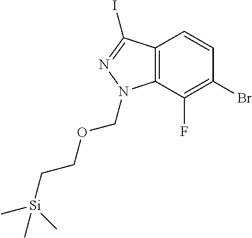 [SMILES: C[Si](C)(C)CCOCn1nc(I)c2ccc(Br)c(F)c21]

A suspension of NaH in mineral oil (3.88 g, 98.8 mmol) was added to a solution of 6-bromo-7-fluoro-3-iodo-1H-indazole (30 g, 88 mmol) in THE (900 mL) at 0° C. The mixture was stirred an hour at 0° C., and 2-(trimethylsilyl)ethoxymethyl chloride (16.6 g, 99.2 mmol) was added at 0° C. After stirring at room temperature an additional two hours, the mixture was quenched with water, and extracted twice with ethyl acetate. The organic layer was washed with brine, and concentrated. The residue was purified by MPLC eluting with a gradient of ethyl acetate in hexanes to afford 6-bromo-7-fluoro-3-iodo-1-((2-(trimethylsilyl)ethoxy)methyl)-1H-indazole (37.3 g, 90%) as colorless oil.

### Part V—Synthesis of Methyl 6-bromo-7-fluoro-1-((2-(trimethylsilyl)ethoxy)methyl)-1H-indazole-3-carboxylate

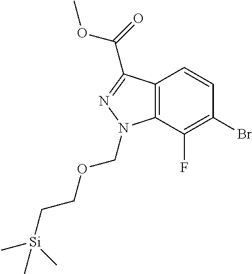 [SMILES: COC(=O)c1nn(COCC[Si](C)(C)C)c2c(F)c(Br)ccc12]

In a 1 L autoclave was placed a suspension of 6-bromo-7-fluoro-3-iodo-1-((2-(trimethylsilyl)ethoxy)methyl)-1H-indazole (10 g, 21.3 mmol), methanol (500 mL), triethyl amine (5.3 g, 7.33 mL, 53 mmol) and [1,1-bis(diphenylphosphino)ferrocene]dichloro-palladium(II) (1.67 g, 2.13 mmoL). The mixture was stirred at 60° C. with a pressure of 5 bars of carbon monoxide overnight. The resulting mixture was concentrated under vacuum. The residue was purified by MPLC eluting with a gradient of ethyl acetate in hexanes to afford methyl 6-bromo-7-fluoro-1-((2-(trimethylsilyl)ethoxy)methyl)-1H-indazole-3-carboxylate (6.84 g, 80%).

### Part VI—Synthesis of Methyl 7-fluoro-6-(4,4,5,5-tetramethyl-1,3,2-dioxaborolan-2-yl)-1-((2-(trimethylsilyl)ethoxy)methyl)-1H-indazole-3-carboxylate

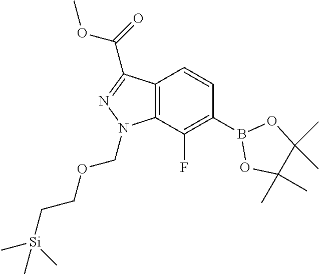

A mixture of 6-bromo-7-fluoro-1-((2-(trimethylsilyl)ethoxy)methyl)-1H-indazole-3-carboxylate (25 g, 62 mmol), potassium acetate (15.2 g, 155 mmol), bis(pinacolato)diboron (1.57 g, 6.2 mmol), [1,1′-bis(diphenylphosphino)ferrocene]dichloropalladium(II) (4.54 g, 6.2 mmol) in dioxane (300 mL) was stirred overnight at 110° C. The reaction mixture was cooled and concentrated. The residue was purified via MPLC eluting with 10% ethyl acetate in petroleum ether to afford methyl 7-fluoro-6-(4,4,5,5-tetramethyl-1,3,2-dioxaborolan-2-yl)-1-((2-(trimethylsilyl)ethoxy)methyl)-1H-indazole-3-carboxylate (26 g, 92%).

### Part VII—Synthesis of Methyl 6-(2,6-difluoro-3-((5-fluoro-2-methoxypyridine)-3-sulfonamido)phenyl)-7-fluoro-1-((2-(trimethylsilyl)ethoxy)methyl)-1H-indazole-3-carboxylate

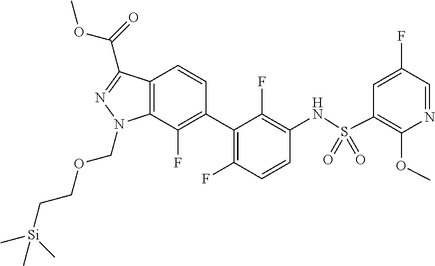

A mixture of methyl 7-fluoro-6-(4,4,5,5-tetramethyl-1,3,2-dioxaborolan-2-yl)-1-((2-(trimethylsilyl)ethoxy)methyl)-1H-indazole-3-carboxylate (1.35 g, 3 mmol) in dioxane (40 mL), water (10 mL), N-(3-bromo-2,4-difluorophenyl)-5-fluoro-2-methoxypyridine-3-sulfonamide (1.0 g, 2.5 mmol), [1,1′-bis(di-tert-butylphosphino)ferrocene]dichloropalladium(II) (162 mg, 0.25 mmol), and potassium phosphate (1.1 g, 5.2 mmol) was stirred at 60° C. overnight. The mixture was concentrated under vacuum. The residue was purified by MPLC eluting with 10% ethyl acetate in petroleum ether to afford methyl 6-(2,6-difluoro-3-((5-fluoro-2-methoxypyridine)-3-sulfonamido)phenyl)-7-fluoro-1-((2-(trimethylsilyl)ethoxy)methyl)-1H-indazole-3-carboxylate (0.61 g, 38%).

### Part VIII and IX—Synthesis of N-(2-Acetamidoethyl)-6-(2,6-difluoro-3-((5-fluoro-2-methoxypyridine)-3-sulfonamido)phenyl)-7-fluoro-1H-indazole-3-carboxamide

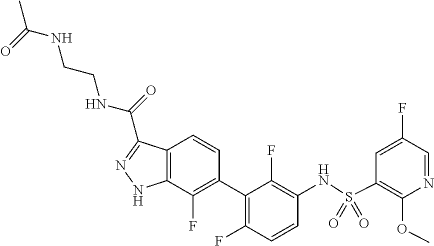 [SMILES: COc1ncc(F)cc1S(=O)(=O)Nc1ccc(F)c(-c2ccc3c(C(=O)NCCNC(C)=O)n[nH]c3c2F)c1F]

N-(2-Acetamidoethyl)-6-(2,6-difluoro-3-((5-fluoro-2-methoxypyridine)-3-sulfonamido)phenyl)-7-fluoro-1H-indazole-3-carboxamide was prepared from methyl 6-(2,6-difluoro-3-((5-fluoro-2-methoxypyridine)-3-sulfonamido)phenyl)-7-fluoro-1-((2-(trimethylsilyl)ethoxy)methyl)-1H-indazole-3-carboxylate using the deprotection procedure described in Part IX of Example 4 and the amide-coupling procedure of Example 5, using N-(2-aminoethyl)acetamide instead of methylamine. ¹H NMR (400 MHz, CD₃OD-d₄) δ 8.28 (s, J=3.0 Hz, 1H), 8.08 (d, J=8.4 Hz, 1H), 7.95 (dd, J=7.2, 3.0 Hz, 1H), 7.60 (td, J=8.9, 5.7 Hz, 1H), 7.19-7.02 (m, 2H), 4.00 (s, 3H), 3.58 (dd, J=6.7, 5.3 Hz, 2H), 3.41-3.54 (m, 2H), 1.98 (s, 3H). (ES, m/z): (M+H)⁺ 581.

### Example 24—Synthesis of 6-(3,5-Difluoro-2-((5-fluoro-2-methylpyridine)-3-sulfonamido)pyridin-4-yl)-7-fluoro-N-methyl-1H-indazole-3-carboxamide

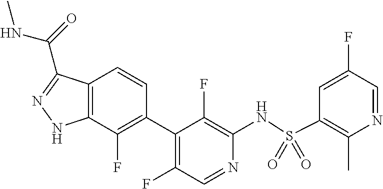 [SMILES: CNC(=O)c1n[nH]c2c(F)c(-c3c(F)cnc(NS(=O)(=O)c4cc(F)cnc4C)c3F)ccc12]

### Part I—Synthesis of N-(3,5-Difluoro-4-iodopyridin-2-yl)-5-fluoro-2-methylpyridine-3-sulfonamide

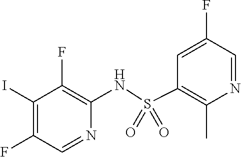 [SMILES: Cc1ncc(F)cc1S(=O)(=O)Nc1ncc(F)c(I)c1F]

A mixture of 2,3,5-trifluoro-4-iodopyridine (204 mg, 0.789 mmol), 5-fluoro-2-methylpyridine-3-sulfonamide (150 mg, 0.789 mmol), potassium carbonate (327 mg, 2.37 mmol), and DMF (5 mL) was stirred overnight at 100° C. The mixture was concentrated, and the residue was purified via MPLC eluting with 12% methanol in dichloromethane/methanol to afford N-(3,5-difluoro-4-iodopyridin-2-yl)-5-fluoro-2-methylpyridine-3-sulfonamide (230 mg, 68%).

### Part II—Synthesis of 6-(3,5-Difluoro-2-((5-fluoro-2-methylpyridine)-3-sulfonamido)pyridin-4-yl)-7-fluoro-1-((2-(trimethylsilyl)ethoxy)methyl)-1H-indazole-3-carboxylic acid

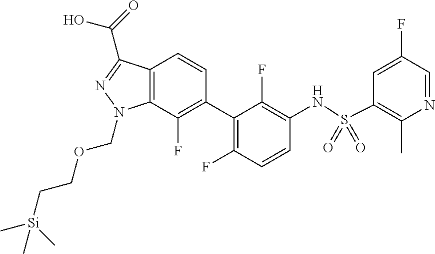 [SMILES: Cc1ncc(F)cc1S(=O)(=O)Nc1ccc(F)c(-c2ccc3c(C(=O)O)nn(COCC[Si](C)(C)C)c3c2F)c1F]

A mixture of N-(3,5-difluoro-4-iodopyridin-2-yl)-5-fluoro-2-methylpyridine-3-sulfonamide (150 mg, 0.35 mmol), methyl 7-fluoro-6-(4,4,5,5-tetramethyl-1,3,2-dioxaborolan-2-yl)-1-((2-(trimethylsilyl)ethoxy)methyl)-1H-indazole-3-carboxylate (210 mg, 0.467 mmol), tetrakis(triphenylphosphine)palladium(0) (54 mg, 0.047 mmol), potassium carbonate (194 mg, 1.401 mmol), toluene (5 mL), ethanol (1.5 mL), and water (1.5 mL) was stirred at 110° C. for three hours. The mixture was concentrated and the residue was purified by reverse phase HPLC eluting with a gradient of 47-53% water in acetonitrile to afford 6-(3,5-difluoro-2-((5-fluoro-2-methylpyridine)-3-sulfonamido)pyridin-4-yl)-7-fluoro-1-((2-(trimethylsilyl)ethoxy)methyl)-1H-indazole-3-carboxylic acid (124 mg, 58%).

### Part III—Synthesis of 6-(3,5-Difluoro-2-((5-fluoro-2-methylpyridine)-3-sulfonamido)pyridin-4-yl)-7-fluoro-N-methyl-1-((2-(trimethylsilyl)ethoxy)methyl)-1H-indazole-3-carboxamide

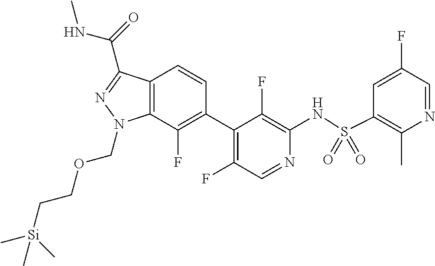 [SMILES: CNC(=O)c1nn(COCC[Si](C)(C)C)c2c(F)c(-c3c(F)cnc(NS(=O)(=O)c4cc(F)cnc4C)c3F)ccc12]

A solution of 6-(3,5-difluoro-2-((5-fluoro-2-methylpyridine)-3-sulfonamido)pyridin-4-yl)-7-fluoro-1-((2-(trimethylsilyl)ethoxy)methyl)-1H-indazole-3-carboxylic acid (105 mg, 0.17 mmol), 1-[bis(dimethylamino)methylene]-1H-1,2,3-triazolo[4,5-b]pyridinium 3-oxid hexafluorophosphate (78 mg, 0.21 mmol), N,N-diisopropylethylamine (111 mg, 0.86 mmol), and DMF (10 mL) was stirred for twenty minutes. A solution of methylamine (8.0 mg, 0.26 mmol) was added and the mixture was stirred at room temperature for three hours and concentrated. The residue was purified via MPLC eluting with a gradient of methanol in dichloromethane to afford 6-(3,5-difluoro-2-((5-fluoro-2-methylpyridine)-3-sulfonamido)pyridin-4-yl)-7-fluoro-N-methyl-1-((2-(trimethylsilyl)ethoxy)methyl)-1H-indazole-3-carboxamide (107 mg, 99%) as a yellow solid.

### Part IV—Synthesis of 6-(3,5-Difluoro-2-((5-fluoro-2-methylpyridine)-3-sulfonamido)pyridin-4-yl)-7-fluoro-N-methyl-1H-indazole-3-carboxamide

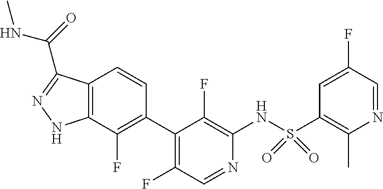 [SMILES: CNC(=O)c1n[nH]c2c(F)c(-c3c(F)cnc(NS(=O)(=O)c4cc(F)cnc4C)c3F)ccc12]

A mixture of 6-(3,5-difluoro-2-((5-fluoro-2-methylpyridine)-3-sulfonamido)pyridin-4-yl)-7-fluoro-N-methyl-1-((2-(trimethylsilyl)ethoxy)methyl)-1H-indazole-3-carboxamide (83 mg, 0.133 mmol), dichloromethane (2 mL), and trifluoroacetic acid (2 mL) was stirred for an hour at room temperature and concentrated. The residue was stirred in 7M ammonia in methanol (1 mL) for an hour at room temperature and concentrated. The residue was purified by reverse phase HPLC eluting with a gradient of 20% to 45% acetonitrile in 0.05% trifluoroacetic acid in water to afford 6-(3,5-difluoro-2-((5-fluoro-2-methylpyridine)-3-sulfonamido)pyridin-4-yl)-7-fluoro-N-methyl-1H-indazole-3-carboxamide (39 mg, 48%) as a white solid. ¹H NMR (400 MHz, DMSO-d₆) δ 1.27 (t, J=6.1 Hz, 1H), 2.46 (s, 3H), 2.87 (s, 3H), 7.04 (dd, J=8.3, 5.6 Hz, 1H), 7.50 (dd, J=8.2, 2.8 Hz, 1H), 7.87 (d, J=8.3 Hz, 1H), 8.06 (d, J=2.8 Hz, 1H), 8.45-8.55 (m, 2H), 10.98 (s, 1H), 14.22 (s, 1H). (ES, m/z): (M+H)⁺ 495.

### Example 25—Synthesis of 7-Fluoro-6-(2-fluoro-3-((5-fluoro-2-methoxypyridine)-3-sulfonamido)-6-methoxyphenyl)-N-(3-hydroxypropyl)-1H-indazole-3-carboxamide

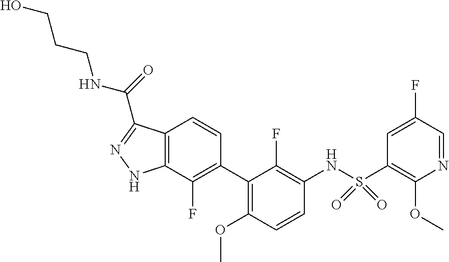 [SMILES: COc1ccc(NS(=O)(=O)c2cc(F)cnc2OC)c(F)c1-c1ccc2c(C(=O)NCCCO)n[nH]c2c1F]

### Part I—Synthesis of 2-Bromo-3-fluoro-1-methoxy-4-nitrobenzene

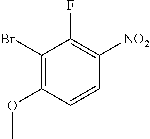

Sodium methoxide (145 mg, 2.68 mmol) was added slowly to a stirred solution of 2-bromo-1,3-difluoro-4-nitrobenzene (637 mg, 2.68 mmol) in methanol (4 mL) at 0° C. The mixture was stirred for an hour at 0° C., and an additional four hours at room temperature. The reaction was diluted with water and was extracted with three times with ethyl acetate. The combined organic layers were dried (Na₂SO₄) and concentrated. The residue was purified via MPLC eluting with 3% ethyl acetate in petroleum ether to afford 2-bromo-3-fluoro-1-methoxy-4-nitrobenzene (306 mg, 46%) as a white solid.

### Part II—Synthesis of Methyl 7-fluoro-6-(2-fluoro-6-methoxy-3-nitrophenyl)-1-((2-(trimethylsilyl)ethoxy)methyl)-1H-indazole-3-carboxylate

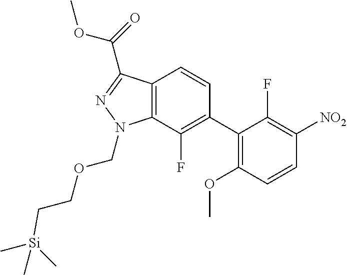 [SMILES: COC(=O)c1nn(COCC[Si](C)(C)C)c2c(F)c(-c3c(OC)ccc([N+](=O)[O-])c3F)ccc12]

A mixture of methyl 7-fluoro-6-(4,4,5,5-tetramethyl-1,3,2-dioxaborolan-2-yl)-1-((2-(trimethylsilyl)ethoxy)methyl)-1H-indazole-3-carboxylate (250 mg, 0.56 mmol), 2-bromo-3-fluoro-1-methoxy-4-nitrobenzene (69 mg, 0.28 mmol), potassium phosphate (295 mg, 1.39 mmol), [1,3-bis(2,6-diisopropylphenyl)imidazol-2-ylidene](3-chloropyridyl)palladium(II) dichloride (38 mg, 0.056 mmol), 1,4-dioxane (5 mL), and water (1 mL) was stirred for three hours at 100° C. The mixture was concentrated, and the residue was purified by MPLC eluting with 33% ethyl acetate in petroleum ether to afford methyl 7-fluoro-6-(2-fluoro-6-methoxy-3-nitrophenyl)-1-((2-(trimethylsilyl)ethoxy)methyl)-1H-indazole-3-carboxylate (137 mg, 50%).

### Part III—Synthesis of 7-Fluoro-6-(2-fluoro-3-((5-fluoro-2-methoxypyridine)-3-sulfonamido)-6-methoxyphenyl)-N-(3-hydroxypropyl)-1H-indazole-3-carboxamide

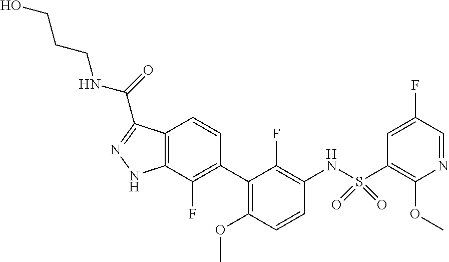 [SMILES: COc1ccc(NS(=O)(=O)c2cc(F)cnc2OC)c(F)c1-c1ccc2c(C(=O)NCCCO)n[nH]c2c1F]

7-Fluoro-6-(2-fluoro-3-((5-fluoro-2-methoxypyridine)-3-sulfonamido)-6-methoxyphenyl)-N-(3-hydroxypropyl)-1H-indazole-3-carboxamide was prepared from methyl 7-fluoro-6-(2-fluoro-6-methoxy-3-nitrophenyl)-1-((2-(trimethylsilyl)ethoxy)methyl)-1H-indazole-3-carboxylate using the procedures described in Part VII-IX of Example 4 and the amide-coupling procedure of Example 5, using 3-aminopropan-1-ol instead of methylamine. ¹H NMR (400 MHz, CD₃OD-d₄ δ 1.89 (p, J=6.5 Hz, 2H), 3.57 (t, J=6.8 Hz, 2H), 3.71 (t, J=6.2 Hz, 2H), 3.78 (s, 3H), 4.01 (s, 3H), 6.93 (dd, J=9.1, 1.5 Hz, 1H), 6.99 (dd, J=8.4, 5.7 Hz, 1H), 7.49 (t, J=8.9 Hz, 1H), 7.88 (dd, J=7.2, 3.0 Hz, 1H), 7.99 (d, J=8.4 Hz, 1H), 8.27 (d, J=3.0 Hz, 1H). (ES, m/z): (M+H)⁺ 566.

### Example 26—Synthesis of 7-Fluoro-6-(2-fluoro-3-((5-fluoro-2-methoxypyridine)-3-sulfonamido)phenyl)-N-(3-hydroxypropyl)-1H-indazole-3-carboxamide

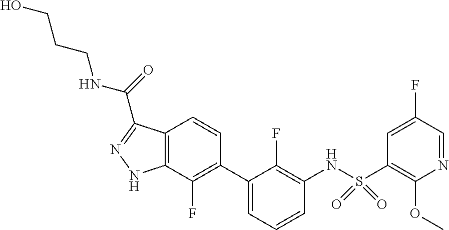 [SMILES: COc1ncc(F)cc1S(=O)(=O)Nc1cccc(-c2ccc3c(C(=O)NCCCO)n[nH]c3c2F)c1F]

### Part I—Synthesis of methyl 7-fluoro-6-(2-fluoro-3-((5-fluoro-2-methoxypyridine)-3-sulfonamido)phenyl)-1-((2-(trimethylsilyl)ethoxy)methyl)-1H-indazole-3-carboxylate

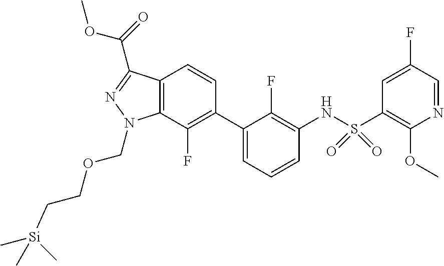

A mixture of N-(3-bromo-2-fluorophenyl)-5-fluoro-2-methoxypyridine-3-sulfonamide (900 mg, 2.37 mmol), 1,4-dioxane (16 mL), water (4 mL), methyl 7-fluoro-6-(4,4,5,5-tetramethyl-1,3,2-dioxaborolan-2-yl)-1-((2-(trimethylsilyl)ethoxy)methyl)-1H-indazole-3-carboxylate (1282 mg, 2.85 mmol), potassium phosphate (1510 mg, 7.12 mmol), [1,1′-bis(di-tert-butylphosphino)ferrocene]dichloropalladium(II) (155 mg, 0.24 mmol) was stirred overnight at 60° C. The mixture was concentrated, and the residue was purified via MPLC eluting with 20% ethyl acetate in petroleum ether to afford methyl 7-fluoro-6-(2-fluoro-3-((5-fluoro-2-methoxypyridine)-3-sulfonamido)phenyl)-1-((2-(trimethylsilyl)ethoxy)methyl)-1H-indazole-3-carboxylate (740 mg, 50%).

### Part II—Synthesis of 7-Fluoro-6-(2-fluoro-3-((5-fluoro-2-methoxypyridine)-3-sulfonamido)phenyl)-1-((2-(trimethylsilyl)ethoxy)methyl)-1H-indazole-3-carboxylic acid

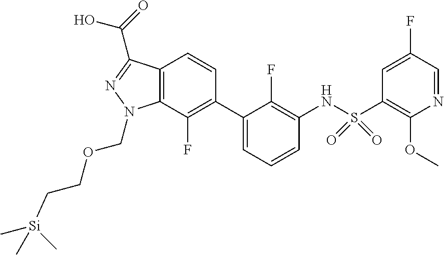

A mixture of methyl 7-fluoro-6-(2-fluoro-3-((5-fluoro-2-methoxypyridine)-3-sulfonamido)phenyl)-1-((2-(trimethylsilyl)ethoxy)methyl)-1H-indazole-3-carboxylate (740 mg, 1.19 mmol), THE (15 mL), water (3 mL), and lithium hydroxide monohydrate (249 mg, 5.94 mmol) was stirred for three hours at room temperature. The pH value of the solution was adjusted to 5-6 with 1M aqueous HCl. The mixture was extracted three times with ethyl acetate. The combined organic layers were washed with brine, dried (Na₂SO₄), and concentrated. The residue was purified via MPLC eluting with 10% methanol in dichloromethane to afford 7-fluoro-6-(2-fluoro-3-((5-fluoro-2-methoxypyridine)-3-sulfonamido)phenyl)-1-((2-(trimethylsilyl)ethoxy)-methyl)-1H-indazole-3-carboxylic acid (450 mg, 62%) as a yellow solid.

### Part III—Synthesis of 7-Fluoro-6-(2-fluoro-3-((5-fluoro-2-methoxypyridine)-3-sulfonamido)phenyl)-N-(3-hydroxypropyl)-1-((2-(trimethylsilyl)ethoxy)methyl)-1H-indazole-3-carboxamide

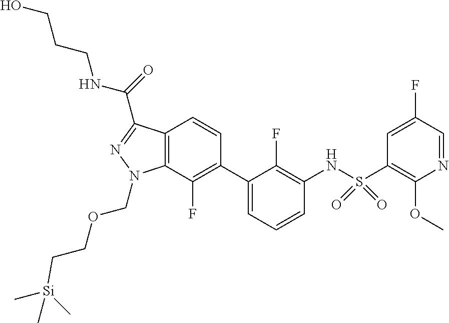

A solution of 7-fluoro-6-(2-fluoro-3-((5-fluoro-2-methoxypyridine)-3-sulfonamido)phenyl)-1-((2-(trimethylsilyl)ethoxy)-methyl)-1H-indazole-3-carboxylic acid (100 mg, 0.164 mmol), dichloromethane (3 mL), 1-[bis(dimethylamino)methylene]-1H-1,2,3-triazolo[4,5-b]pyridinium 3-oxid hexafluorophosphate (125 mg, 0.329 mmol), N,N-diisopropylethylamine (106 mg, 0.82 mmol), DMF (0.60 mL), and 3-aminopropan-1-ol (12.34 mg, 0.164 mmol) was stirred for three hours at room temperature and concentrated. The residue was purified via MPLC eluting with 5% methanol in dichloromethane to afford 7-fluoro-6-(2-fluoro-3-((5-fluoro-2-methoxypyridine)-3-sulfonamido)phenyl)-N-(3-hydroxypropyl)-1-((2-(trimethylsilyl)ethoxy)methyl)-1H-indazole-3-carboxamide (80 mg, 73%) as a yellow solid.

### Part IV—Synthesis of 7-Fluoro-6-(2-fluoro-3-((5-fluoro-2-methoxypyridine)-3-sulfonamido)phenyl)-N-(3-hydroxypropyl)-1H-indazole-3-carboxamide

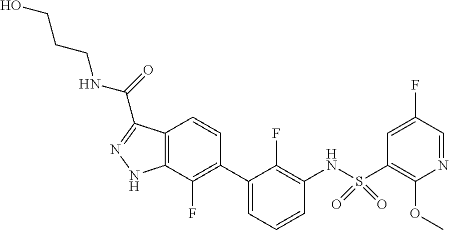 [SMILES: COc1ncc(F)cc1S(=O)(=O)Nc1cccc(-c2ccc3c(C(=O)NCCCO)n[nH]c3c2F)c1F]

A mixture of 7-fluoro-6-(2-fluoro-3-((5-fluoro-2-methoxypyridine)-3-sulfonamido)phenyl)-N-(3-hydroxypropyl)-1-((2-(trimethylsilyl)ethoxy)methyl)-1H-indazole-3-carboxamide (60 mg, 0.090 mmol), dichloromethane (4 mL), and trifluoroacetic acid (2 mL) was stirred for two hours at room temperature and concentrated. The residue purified by reverse phase HPLC eluting with a gradient of 25% to 40% acetonitrile in 0.05% trifluoroacetic acid in water to afford 7-fluoro-6-(2-fluoro-3-((5-fluoro-2-methoxypyridine)-3-sulfonamido)phenyl)-N-(3-hydroxypropyl)-1H-indazole-3-carboxamide (20.6 mg, 43%) as a white solid. ¹H NMR (400 MHz, CD₃OD) δ 8.21 (d, J=3.0 Hz, 1H), 8.05 (d, J=8.4 Hz, 1H), 7.97 (dd, J=7.3, 3.0 Hz, 1H), 7.51 (m, 1H), 7.23-7.12 (m, 3H), 3.97 (s, 3H), 3.71 (t, J=6.2 Hz, 2H), 3.57 (t, J=6.8 Hz, 2H), 1.89 (p, J=6.5 Hz, 2H). (ES, m/z): (M+H)⁺ 536.

### Example 27—Synthesis of N-(2,5-Difluoro-3-(7-fluoro-3-(1H-imidazol-2-yl)-1H-indazol-6-yl)phenyl)-5-fluoro-2-methylpyridine-3-sulfonamide

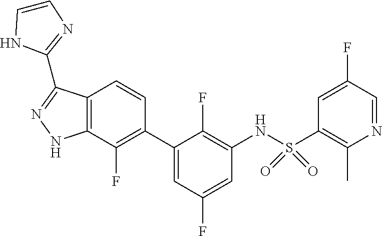 [SMILES: Cc1ncc(F)cc1S(=O)(=O)Nc1cc(F)cc(-c2ccc3c(-c4ncc[nH]4)n[nH]c3c2F)c1F]

### Part I—Synthesis of 2,5-Difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)aniline

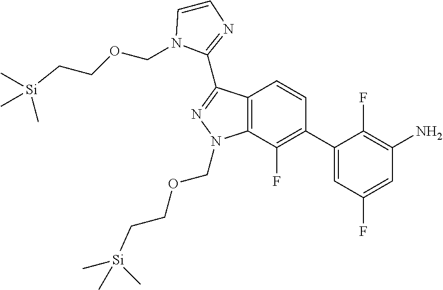 [SMILES: C[Si](C)(C)CCOCn1ccnc1-c1nn(COCC[Si](C)(C)C)c2c(F)c(-c3cc(F)cc(N)c3F)ccc12]

A mixture of 7-fluoro-6-(4,4,5,5-tetramethyl-1,3,2-dioxaborolan-2-yl)-1-((2-(trimethylsilyl)ethoxy)methyl)-3-(1-((2-(trimethylsilyl)ethoxy)methyl)-1H-imidazol-2-yl)-1H-indazole (500 mg, 0.849 mmol), 1,4-dioxane (6 mL), water (2 mL), potassium carbonate (352 mg, 2.55 mmol), [1,1′-bis(diphenylphosphino)ferrocene]dichloropalladium(II) (62 mg, 0.085 mmol), and 3-bromo-2,5-difluoroaniline (177 mg, 0.85 mmol) was stirred for two hours at 90° C. The mixture was cooled and extracted twice with ethyl acetate. The combined organic layers were dried (Na₂SO₄) and concentrated. The residue was purified via MPLC eluting with 33% ethyl acetate in petroleum ether to afford 2,5-difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)aniline (300 mg, 60%) as a yellow solid.

### Part II—Synthesis of N-(2,5-Difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)phenyl)-5-fluoro-2-methylpyridine-3-sulfonamide

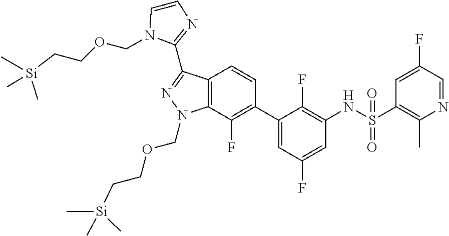 [SMILES: Cc1ncc(F)cc1S(=O)(=O)Nc1cc(F)cc(-c2ccc3c(-c4nccn4COCC[Si](C)(C)C)nn(COCC[Si](C)(C)C)c3c2F)c1F]

5-Fluoro-2-methylpyridine-3-sulfonyl chloride (70 mg, 0.33 mmol) was added dropwise to a stirred solution of 2,5-difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)aniline (100 mg, 0.17 mmol) and pyridine (3 mL) at 0-5° C. The solution was stirred overnight at room temperature and concentrated. The residue was purified via MPLC eluting a gradient of 2-5% methanol in dichloromethane to afford N-(2,5-difluoro-3-(7-fluoro-1-((2-(trimethylsilyl)ethoxy)-methyl)-3-(1-((2-(trimethylsilyl)ethoxy)methyl)-1H-imidazol-2-yl)-1H-indazol-6-yl)phenyl)-5-fluoro-2-methylpyridine-3-sulfonamide (90 mg, 70%).

### Part III—Synthesis of N-(2,5-Difluoro-3-(7-fluoro-3-(1H-imidazol-2-yl)-1H-indazol-6-yl)phenyl)-5-fluoro-2-methylpyridine-3-sulfonamide

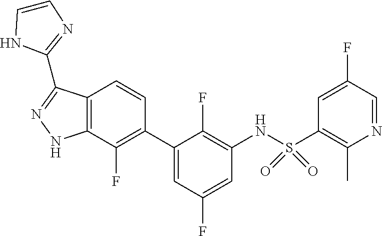 [SMILES: Cc1ncc(F)cc1S(=O)(=O)Nc1cc(F)cc(-c2ccc3c(-c4ncc[nH]4)n[nH]c3c2F)c1F]

A mixture of 7-fluoro-6-(2-fluoro-3-((5-fluoro-2-methoxypyridine)-3-sulfonamido)phenyl)-N-(3-hydroxypropyl)-1-((2-(trimethylsilyl)ethoxy)methyl)-1H-indazole-3-carboxamide (90 mg, 0.12 mmol) and trifluoroacetic acid (3 mL) was stirred for an hour at room temperature and concentrated. The residue was stirred in 7M ammonia in methanol (5 mL) for thirty minutes at room temperature and concentrated. The residue was purified by reverse phase HPLC eluting with a gradient of 17% to 40% acetonitrile in 0.05% trifluoroacetic acid in water to afford N-(2,5-difluoro-3-(7-fluorxzo-3-(1H-imidazol-2-yl)-1H-indazol-6-yl)phenyl)-5-fluoro-2-methylpyridine-3-sulfonamide (41.5 mg, 76%) as a white solid. ¹H NMR (400 MHz, CD₃OD) δ 8.58 (d, J=2.8 Hz, 1H), 7.97-8.09 (m, 2H), 7.72 (s, 2H), 7.41 (m, 1H), 7.31 (dd, J=8.5, 5.8 Hz, 1H), 7.17 (ddd, J=8.3, 5.1, 3.1 Hz, 1H), 2.85 (s, 3H). (ES, m/z): (M+H)⁺ 503.

### Example 28—Synthesis of N-(3,5-Difluoro-4-(7-fluoro-3-(1H-imidazol-2-yl)-1H-indazol-6-yl)pyridin-2-yl)-5-fluoro-2-methoxypyridine-3-sulfonamide

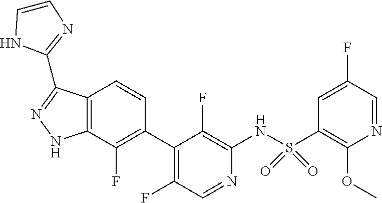 [SMILES: COc1ncc(F)cc1S(=O)(=O)Nc1ncc(F)c(-c2ccc3c(-c4ncc[nH]4)n[nH]c3c2F)c1F]

### Part I—Synthesis of 3,5-Difluoro-4-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)pyridin-2-amine

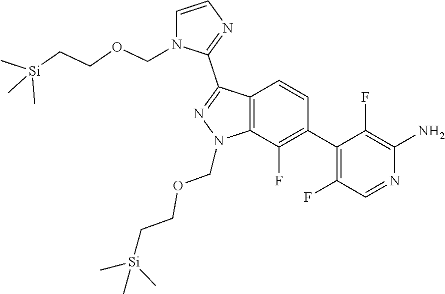 [SMILES: C[Si](C)(C)CCOCn1ccnc1-c1nn(COCC[Si](C)(C)C)c2c(F)c(-c3c(F)cnc(N)c3F)ccc12]

A mixture of 7-fluoro-6-(4,4,5,5-tetramethyl-1,3,2-dioxaborolan-2-yl)-1-((2-(trimethylsilyl)ethoxy)methyl)-3-(1-((2-(trimethylsilyl)ethoxy)methyl)-1H-imidazol-2-yl)-1H-indazole (1.00 g, 1.70 mmol), 3,5-difluoro-4-iodopyridin-2-amine (0.43 g, 2.0 mmol), potassium carbonate (0.70 g, 5.0 mmol), [1,1′-bis(diphenylphosphino)ferrocene]dichloro-palladium(II) (0.06 g, 0.000 mmol), water (5 mL), and 1,4-dioxane (20 mL) was stirred for two hours at 90° C. The mixture was concentrated, and the residue was purified via MPLC eluting with 50% ethyl acetate in petroleum ether to afford 3,5-difluoro-4-(7-fluoro-1-((2-(trimethylsilyl)ethoxy)methyl)-3-(1-((2-(trimethylsilyl)ethoxy)-methyl)-1H-imidazol-2-yl)-1H-indazol-6-yl)pyridin-2-amine (800 mg, 80%) as a yellow oil.

### Part II—Synthesis of 6-(2-Bromo-3,5-difluoropyridin-4-yl)-7-fluoro-1-((2-(trimethylsilyl)ethoxy)methyl)-3-(1-((2-(trimethylsilyl)ethoxy)methyl)-1H-imidazol-2-yl)-1H-indazole

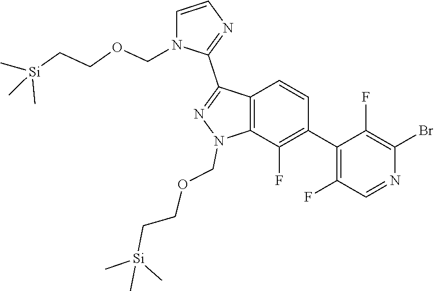 [SMILES: C[Si](C)(C)CCOCn1ccnc1-c1nn(COCC[Si](C)(C)C)c2c(F)c(-c3c(F)cnc(Br)c3F)ccc12]

A mixture of 3,5-difluoro-4-(7-fluoro-1-((2-(trimethylsilyl)ethoxy)methyl)-3-(1-((2-(trimethylsilyl)ethoxy)-methyl)-1H-imidazol-2-yl)-1H-indazol-6-yl)pyridin-2-amine (600 mg, 1.0 mmol), copper(II) bromide (272 mg, 1.22 mmol), acetonitrile (10 mL), tert-butyl nitrite (154 mg, 1.5 mmol) was stirred for an hour at 65° C. The mixture was concentrated, and the residue was purified via MPLC eluting with 20% ethyl acetate in petroleum ether to afford 6-(2-bromo-3,5-difluoropyridin-4-yl)-7-fluoro-1-((2-(trimethylsilyl)ethoxy)methyl)-3-(1-((2-(trimethylsilyl)ethoxy)methyl)-1H-imidazol-2-yl)-1H-indazole (300 mg, 45%) as a yellow oil.

### Part III—Synthesis of N-(3,5-Difluoro-4-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)pyridin-2-yl)-5-fluoro-2-methoxypyridine-3-sulfonamide

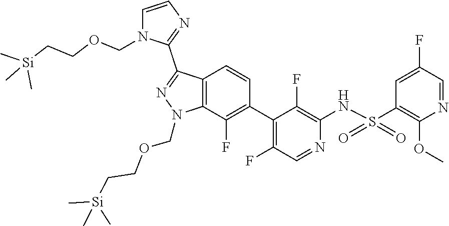

A mixture of 6-(2-bromo-3,5-difluoropyridin-4-yl)-7-fluoro-1-((2-(trimethylsilyl)ethoxy)methyl)-3-(1-((2-(trimethylsilyl)ethoxy)methyl)-1H-imidazol-2-yl)-1H-indazole (100 mg, 0.153 mmol), (1R,2R)—N′,N′-dimethylcyclohexane-1,2-diamine (6.52 mg, 0.046 mmol), 5-fluoro-2-methoxypyridine-3-sulfonamide (31 mg, 0.15 mmol), potassium carbonate (42 mg, 0.305 mmol), copper(I) iodide (4.36 mg, 0.023 mmol), and DMF (5 mL) was stirred overnight at 120° C. The mixture was concentrated, and the residue was purified via MPLC eluting with 16% methanol in dichloromethane to afford N-(3,5-difluoro-4-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)pyridin-2-yl)-5-fluoro-2-methoxypyridine-3-sulfonamide (50 mg, 42%) as a yellow solid.

### Part IV—Synthesis of N-(3,5-Difluoro-4-(7-fluoro-3-(1H-imidazol-2-yl)-1H-indazol-6-yl)pyridin-2-yl)-5-fluoro-2-methoxypyridine-3-sulfonamide

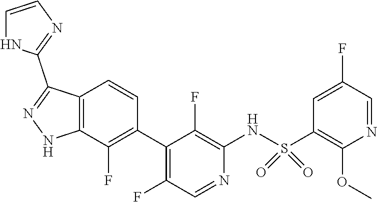 [SMILES: COc1ncc(F)cc1S(=O)(=O)Nc1ncc(F)c(-c2ccc3c(-c4ncc[nH]4)n[nH]c3c2F)c1F]

A solution of N-(3,5-difluoro-4-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)pyridin-2-yl)-5-fluoro-2-methoxypyridine-3-sulfonamide (50 mg, 0.064 mmol) and trifluoroacetic acid (2 mL) was stirred for an hour at room temperature and concentrated. The residue was stirred in 7M ammonia in methanol (2 mL) for an hour at room temperature and concentrated. The residue was purified by reverse phase HPLC eluting with a gradient of 10% to 30% acetonitrile in water with 0.05% trifluoroacetic acid to afford N-(3,5-difluoro-4-(7-fluoro-3-(1H-imidazol-2-yl)-1H-indazol-6-yl)pyridin-2-yl)-5-fluoro-2-methoxypyridine-3-sulfonamide (12.2 mg, 36%) as a white solid. ¹H NMR (400 MHz, CD₃OD-d₄) δ 8.23 (s, 1H), 8.20 (m, 1H), 8.14 (d, 1H), 7.98 (s, 1H), 7.26 (s, 2H), 7.23 (m, 1H), 4.01 (s, 3H). (ES, m/z): (M+H)⁺ 520.

### Example 29—Synthesis of N-(3-Cyano-2-(7-fluoro-3-(1H-imidazol-2-yl)-1H-indazol-6-yl)pyridin-4-yl)-5-fluoro-2-methoxypyridine-3-sulfonamide

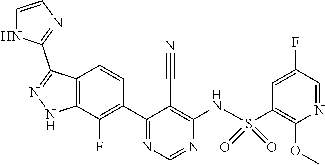 [SMILES: COc1ncc(F)cc1S(=O)(=O)Nc1ncnc(-c2ccc3c(-c4ncc[nH]4)n[nH]c3c2F)c1C#N]

### Part I—Synthesis of 4-Amino-2-bromonicotinonitrile

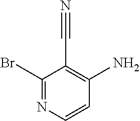

A mixture of 2,4-dibromopyridine-3-carbonitrile (2.50 g, 9.55 mmol), THE (20 mL), and ammonium hydroxide (20 mL) was stirred for two hours at 100° C. The mixture was concentrated, then diluted with water (100 mL). The mixture was extracted three times with ethyl acetate, and the combined organic layers were concentrated. The residue was purified by re-crystallization from dichloromethane to afford 4-amino-2-bromonicotinonitrile (1.25 g, 66%) as a light yellow solid. The mother liquor from the recrystallization was concentrated to afford 2-amino-4-bromonicotinonitrile (860 mg, 28%) as a light yellow solid.

### Part II—Synthesis of N-(2-bromo-3-cyanopyridin-4-yl)-5-fluoro-2-methoxypyridine-3-sulfonamide

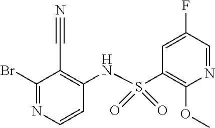 [SMILES: COc1ncc(F)cc1S(=O)(=O)Nc1ccnc(Br)c1C#N]

Sodium hydride (808 mg, 34 mmol) was added to a solution of 4-amino-2-bromopyridine-3-carbonitrile (1.00 g, 5.05 mmol) in THE (15 mL) at 0° C. The mixture was stirred for thirty minutes at 0° C., and then 5-fluoro-2-methoxypyridine-3-sulfonyl chloride (1.48 g, 6.56 mmol) was added with stirring at 0° C. The resulting solution was stirred for an additional two hours at room temperature. The mixture was quenched by the addition of ice, and the mixture was concentrated. The residue was purified via MPLC eluting with 10% ethyl acetate in petroleum ether to afford N-(2-bromo-3-cyanopyridin-4-yl)-5-fluoro-2-methoxypyridine-3-sulfonamide (920 mg, 47%).

### Part III—Synthesis of N-(3-Cyano-2-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)pyridin-4-yl)-5-fluoro-2-methoxypyridine-3-sulfonamide

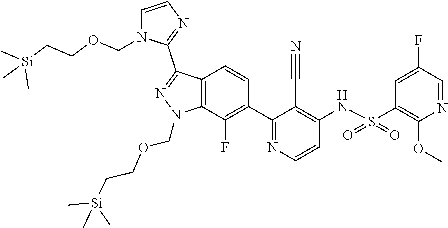

A mixture of N-(2-bromo-3-cyanopyridin-4-yl)-5-fluoro-2-methoxypyridine-3-sulfonamide (300 mg, 0.775 mmol), 1,4-dioxane (5 mL), water (0.5 mL), 7-fluoro-6-(4,4,5,5-tetramethyl-1,3,2-dioxaborolan-2-yl)-1-((2-(trimethylsilyl)ethoxy)methyl)-3-(1-((2-(trimethylsilyl)ethoxy)methyl)-1H-imidazol-2-yl)-1H-indazole (479 mg, 0.814 mmol), potassium carbonate (321 mg, 2.32 mmol), and [1,1′-bis(diphenylphosphino)ferrocene]dichloro-palladium(II) (57 mg, 0.077 mmol) was stirred for two hours at 90° C. The reaction mixture was cooled, filtered, and the filtrate was concentrated. The residue was purified via MPLC eluting with 25% ethyl acetate in petroleum ether to afford N-(3-cyano-2-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)pyridin-4-yl)-5-fluoro-2-methoxypyridine-3-sulfonamide (120 mg, 20%) as a light yellow solid.

### Part IV—Synthesis of N-(3-Cyano-2-(7-fluoro-3-(1H-imidazol-2-yl)-1H-indazol-6-yl)pyridin-4-yl)-5-fluoro-2-methoxypyridine-3-sulfonamide

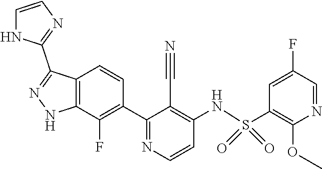 [SMILES: COc1ncc(F)cc1S(=O)(=O)Nc1ccnc(-c2ccc3c(-c4ncc[nH]4)n[nH]c3c2F)c1C#N]

A solution of N-(3-cyano-2-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)pyridin-4-yl)-5-fluoro-2-methoxypyridine-3-sulfonamide (120 mg, 0.156 mmol) and trifluoroacetic acid (3 mL) was stirred for an hour at room temperature and concentrated. The residue was stirred in 7M ammonia in methanol (5 mL) for thirty minutes at room temperature and concentrated. The residue was purified by reverse phase HPLC eluting with a gradient of 15% to 23% acetonitrile in water with 0.05% trifluoroacetic acid to afford N-(3-cyano-2-(7-fluoro-3-(1H-imidazol-2-yl)-1H-indazol-6-yl)pyridin-4-yl)-5-fluoro-2-methoxypyridine-3-sulfonamide (35.5 mg, 45%) as a white solid. ¹H NMR (400 MHz, CD₃OD-d₄) δ 8.27 (d, J=3.1 Hz, 2H), 8.15-8.23 (m, 2H), 7.88 (d, J=7.3 Hz, 1H), 7.77 (s, 2H), 7.62 (dd, J=8.5, 5.7 Hz, 1H), 3.99 (s, 3H). (ES, m/z): (M+H)⁺ 509.

### Example 30—Synthesis of N-(3,5-Difluoro-4-(7-fluoro-3-(1H-imidazol-2-yl)-1H-indazol-6-yl)pyridin-2-yl)-5-fluoro-2-methylbenzenesulfonamide

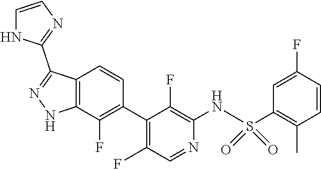 [SMILES: Cc1ccc(F)cc1S(=O)(=O)Nc1ncc(F)c(-c2ccc3c(-c4ncc[nH]4)n[nH]c3c2F)c1F]

### Part I—Synthesis of N-(3,5-Difluoro-4-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)pyridin-2-yl)-5-fluoro-2-methylbenzenesulfonamide

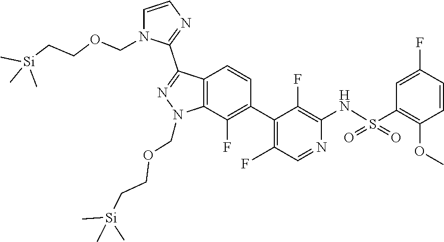

Sodium hydride (15.64 mg, 0.652 mmol) was added to a solution of 3,5-difluoro-4-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)pyridin-2-amine (350 mg, 0.592 mmol) in THE (20 mL) at 0° C. and was stirred for thirty minutes. 5-Fluoro-2-methylbenzenesulfonyl chloride (148 mg, 0.711 mmol) was added and was stirred overnight at room temperature. Water (10 mL) was added and the mixture was extracted three times with ethyl acetate (10 mL each). The combined organic layers were dried (Na₂SO₄) and concentrated. The residue was purified via MPLC eluting with 33% ethyl acetate in petroleum ether to afford N-(3,5-difluoro-4-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)pyridin-2-yl)-5-fluoro-2-methylbenzenesulfonamide (88 mg, 19%).

### Part II—Synthesis of N-(3,5-Difluoro-4-(7-fluoro-3-(1H-imidazol-2-yl)-1H-indazol-6-yl)pyridin-2-yl)-5-fluoro-2-methylbenzenesulfonamide

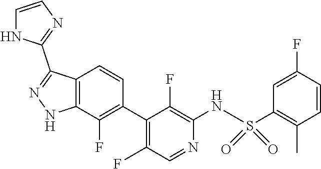 [SMILES: Cc1ccc(F)cc1S(=O)(=O)Nc1ncc(F)c(-c2ccc3c(-c4ncc[nH]4)n[nH]c3c2F)c1F]

A solution of N-(3,5-difluoro-4-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)pyridin-2-yl)-5-fluoro-2-methylbenzenesulfonamide (50 mg, 0.064 mmol) and trifluoroacetic acid (2 mL) was stirred for an hour at room temperature and concentrated. The residue was stirred in 7M ammonia in methanol (2 mL) for an hour at room temperature and concentrated. The residue was purified by reverse phase HPLC eluting with a gradient of 40% to 60% acetonitrile in 10 mM ammonium carbonate to afford N-(3,5-difluoro-4-(7-fluoro-3-(1H-imidazol-2-yl)-1H-indazol-6-yl)pyridin-2-yl)-5-fluoro-2-methylbenzenesulfonamide (25 mg, 43%) as a white solid. ¹H NMR (400 MHz, CD₃OD-d₄) δ 8.24 (dd, 1H), 8.01 (s, 1H), 7.85 (dd, 1H), 7.35 (m, 1H), 7.29 (s, 2H), 7.25 (m, 2H), 2.69 (s, 3H). (ES, m/z): (M+H)⁺ 503.

### Example 31—Synthesis of N-(3-Cyano-4-(7-fluoro-3-(1H-imidazol-2-yl)-1H-indazol-6-yl)pyridin-2-yl)-5-fluoro-2-methoxypyridine-3-sulfonamide

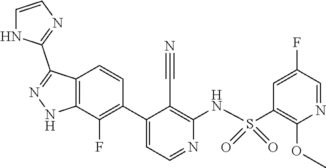 [SMILES: COc1ncc(F)cc1S(=O)(=O)Nc1nccc(-c2ccc3c(-c4ncc[nH]4)n[nH]c3c2F)c1C#N]

### Part I—Synthesis of N-(4-Bromo-3-cyanopyridin-2-yl)-5-fluoro-2-methoxypyridine-3-sulfonamide

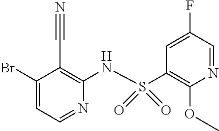 [SMILES: COc1ncc(F)cc1S(=O)(=O)Nc1nccc(Br)c1C#N]

Sodium hydride (397 mg, 16.5 mmol) was added to a solution of 2-amino-4-bromopyridine-3-carbonitrile (560 mg, 2.83 mmol) in THE (15 mL) at 0° C. The mixture was stirred for ten minutes at 0° C., and then 5-fluoro-2-methoxypyridine-3-sulfonyl chloride (490 mg, 2.17 mmol) was added. The resulting solution was stirred for an additional two hours at room temperature. The mixture was quenched by the addition of ice, and the mixture was concentrated. The residue was purified via MPLC eluting with 10% ethyl acetate in petroleum ether to afford N-(4-bromo-3-cyanopyridin-2-yl)-5-fluoro-2-methoxypyridine-3-sulfonamide (370 mg, 34%).

### Part II—Synthesis of N-(3-Cyano-4-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)pyridin-2-yl)-5-fluoro-2-methoxypyridine-3-sulfonamide

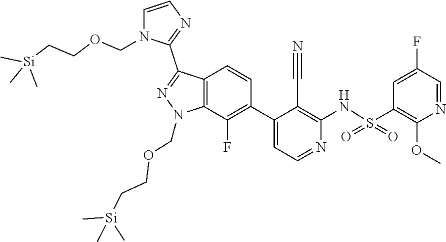

A mixture of N-(4-bromo-3-cyanopyridin-2-yl)-5-fluoro-2-methoxypyridine-3-sulfonamide (200 mg, 0.52 mmol), toluene (10 mL), ethanol (3 mL), water (3 mL), 7-fluoro-6-(4,4,5,5-tetramethyl-1,3,2-dioxaborolan-2-yl)-1-((2-(trimethylsilyl)ethoxy)methyl)-3-(1-((2-(trimethylsilyl)ethoxy)methyl)-1H-imidazol-2-yl)-1H-indazole (319 mg, 0.542 mmol), potassium carbonate (143 mg, 1.03 mmol), and tetrakis(triphenylphosphine)palladium(0) (30 mg, 0.026 mmol) was stirred for two hours at 110° C. The reaction mixture was cooled, filtered, and the filtrate was concentrated. The residue was purified via MPLC eluting with 16% ethyl acetate in petroleum ether to afford N-(3-cyano-4-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)pyridin-2-yl)-5-fluoro-2-methoxypyridine-3-sulfonamide (230 mg, 58%) as a light yellow solid.

### Part III—Synthesis of N-(3-Cyano-2-(7-fluoro-3-(1H-imidazol-2-yl)-1H-indazol-6-yl)pyridin-4-yl)-5-fluoro-2-methoxypyridine-3-sulfonamide

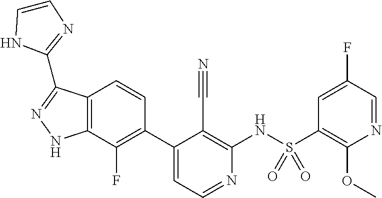 [SMILES: COc1ncc(F)cc1S(=O)(=O)Nc1nccc(-c2ccc3c(-c4ncc[nH]4)n[nH]c3c2F)c1C#N]

A solution of N-(3-cyano-4-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)pyridin-2-yl)-5-fluoro-2-methoxypyridine-3-sulfonamide (170 mg) and trifluoroacetic acid (3 mL) was stirred for an hour at room temperature and concentrated. The residue was stirred in 7M ammonia in methanol (5 mL) for thirty minutes at room temperature and concentrated. The residue was purified by reverse phase HPLC eluting with a gradient of 5% to 40% acetonitrile in water with 0.05% trifluoroacetic acid to afford N-(3-cyano-2-(7-fluoro-3-(1H-imidazol-2-yl)-1H-indazol-6-yl)pyridin-4-yl)-5-fluoro-2-methoxypyridine-3-sulfonamide (71 mg, 63%) as a white solid. ¹H NMR (400 MHz, CD₃OD-d₄) δ 8.26-8.34 (m, 2H), 8.12-8.20 (m, 2H), 7.75 (s, 2H), 7.52 (dd, J=8.5, 5.9 Hz, 1H), 7.22 (d, J=6.2 Hz, 1H), 3.95 (s, 3H). (ES, m/z): (M+H)⁺ 509.

### Example 32—Synthesis of N-(2,4-Difluoro-3-(7-fluoro-3-(1H-imidazol-2-yl)-1H-indazol-6-yl)phenyl)-5-fluoro-3-(hydroxymethyl)-2-methylbenzenesulfonamide

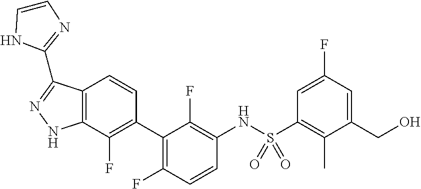 [SMILES: Cc1c(CO)cc(F)cc1S(=O)(=O)Nc1ccc(F)c(-c2ccc3c(-c4ncc[nH]4)n[nH]c3c2F)c1F]

### Part I—Synthesis of Methyl 3-amino-5-fluoro-2-methylbenzoate

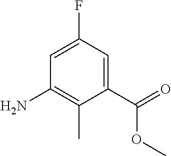

A mixture of methyl 5-fluoro-2-methyl-3-nitrobenzoate (2.00 g, 9.38 mmol), propan-2-ol (18 mL), water (2 mL), ammonium chloride (854 mg, 16.0 mmol), and iron powder (1.30 g, 0.023 mmol) was stirred for five hours at 80° C. The mixture was filtered, and the filtrate was concentrated. The residue was purified via MPLC eluting with a gradient of 10-20% ethyl acetate in petroleum ether to afford methyl 3-amino-5-fluoro-2-methylbenzoate (1.6 g, 93%) as a light yellow oil.

### Part II—Synthesis of Methyl 3-(chlorosulfonyl)-5-fluoro-2-methylbenzoate

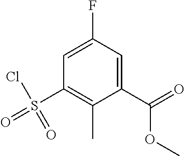 [SMILES: COC(=O)c1cc(F)cc(S(=O)(=O)Cl)c1C]

Sulfonyl chloride (2.50 mL, 1 equiv) was added dropwise with stirring to H₂O (15.00 mL) at 0° C. The mixture was stirred overnight at room temperature. To this was added CuCl (25.00 mg) at 0° C. The resulting solution was stirred for 10 min at 0° C. to obtain “solution A”. Into a 100-mL round-bottom flask, was placed methyl 3-amino-5-fluoro-2-methylbenzoate (500.00 mg, 2.730 mmol, 1.00 equiv) and HCl (3.00 mL). This was followed by the addition of a solution of NaNO₂ (282.50 mg, 4.094 mmol, 1.50 equiv) in H₂O (0.5 mL) dropwise with stirring at 0° C. over 30 min. The above mixture was stirred for 0.5 hour at 0° C. to give “mixture B”. Supernatant of mixture B was added to solution A at 0° C. over 30 minutes. The resulting solution was stirred for 30 min at 0° C. in an ice/salt bath. The resulting solution was extracted with 3×50 mL of dichloromethane, and the organic layers were combined and concentrated to afford methyl 3-(chlorosulfonyl)-5-fluoro-2-methylbenzoate (630 mg, 86.6%) as a light yellow oil.

### Part III—Synthesis of Methyl 3-(N-(2,4-difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)phenyl)sulfamoyl)-5-fluoro-2-methylbenzoate

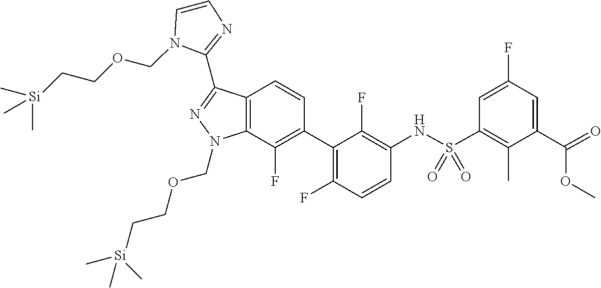 [SMILES: COC(=O)c1cc(F)cc(S(=O)(=O)Nc2ccc(F)c(-c3ccc4c(-c5nccn5COCC[Si](C)(C)C)nn(COCC[Si](C)(C)C)c4c3F)c2F)c1C]

A mixture of 2,4-difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)aniline (300 mg, 0.51 mol), pyridine (2 mL), and methyl 3-(chlorosulfonyl)-5-fluoro-2-methylbenzoate (203 mg, 0.76 mmol) was stirred overnight at room temperature and concentrated. The residue was purified via MPLC eluting with a gradient of 2-5% methanol in dichloromethane to afford methyl 3-(N-(2,4-difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)phenyl)sulfamoyl)-5-fluoro-2-methylbenzoate (320 mg, 77%) as a light yellow oil.

### Part IV—Synthesis of Methyl 3-(N-(2,4-difluoro-3-(7-fluoro-3-(1H-imidazol-2-yl)-1H-indazol-6-yl)phenyl)sulfamoyl)-5-fluoro-2-methylbenzoate

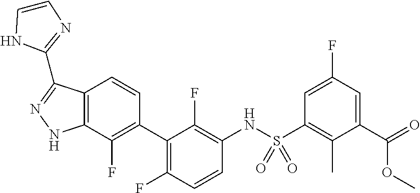 [SMILES: COC(=O)c1cc(F)cc(S(=O)(=O)Nc2ccc(F)c(-c3ccc4c(-c5ncc[nH]5)n[nH]c4c3F)c2F)c1C]

A solution of methyl 3-(N-(2,4-difluoro-3-(7-fluoro-1-((2-(trimethylsilyl)ethoxy)methyl)-3-(1-((2-(trimethylsilyl)ethoxy)methyl)-1H-imidazol-2-yl)-1H-indazol-6-yl)phenyl)sulfamoyl)-5-fluoro-2-methylbenzoate (320 mg, 0.39 mmol) in trifluoroacetic acid (3 mL) was stirred for an hour at room temperature and concentrated. The residue was purified via MPLC eluting with a gradient of 2-5% methanol in dichloromethane to afford methyl 3-(N-(2,4-difluoro-3-(7-fluoro-3-(1H-imidazol-2-yl)-1H-indazol-6-yl)phenyl)sulfamoyl)-5-fluoro-2-methylbenzoate (180 mg, 82%) as a light yellow solid.

### Part V—Synthesis of N-(2,4-Difluoro-3-(7-fluoro-3-(1H-imidazol-2-yl)-1H-indazol-6-yl)phenyl)-5-fluoro-3-(hydroxymethyl)-2-methylbenzenesulfonamide

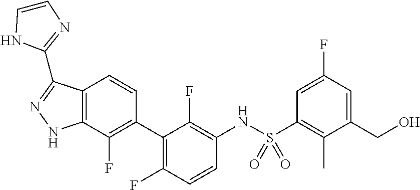 [SMILES: Cc1c(CO)cc(F)cc1S(=O)(=O)Nc1ccc(F)c(-c2ccc3c(-c4ncc[nH]4)n[nH]c3c2F)c1F]

A mixture of methyl 3-(N-(2,4-difluoro-3-(7-fluoro-3-(1H-imidazol-2-yl)-1H-indazol-6-yl)phenyl)sulfamoyl)-5-fluoro-2-methylbenzoate (300 mg, 0.536 mmol), THE (15 mL), and lithium aluminum hydride (50 mg, 1.32 mmol) was stirred for an hour at room temperature. Water (20 μL) was added, and the mixture was filtered. The filtrate was concentrated, and the residue was purified by Prep-HPLC eluting with a gradient of 20-48% acetonitrile in water with 0.05% trifluoroacetic acid to afford N-(2,4-difluoro-3-(7-fluoro-3-(1H-imidazol-2-yl)-1H-indazol-6-yl)phenyl)-5-fluoro-3-(hydroxymethyl)-2-methylbenzenesulfonamide (35.6 mg, 12%) as a white solid. ¹H NMR (400 MHz, CD₃OD-d₄) δ 8.02 (d, J=8.6 Hz, 1H), 7.75 (s, 2H), 7.47-7.57 (m, 3H), 7.26 (dd, J=8.5, 5.8 Hz, 1H), 7.14 (td, J=9.0, 1.8 Hz, 1H), 4.68 (s, 2H), 2.56 (s, 3H). (ES, m/z): (M+H)⁺ 532.

### Example 33—Synthesis of Imidazo[1,2-a]pyridine-8-sulfonyl chloride Intermediate

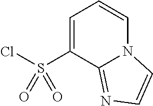 [SMILES: O=S(=O)(Cl)c1cccn2ccnc12]

A mixture of imidazo[1,2-a]pyridine (1.20 g, 10.2 mmol) and chlorosulfonic acid (6.0 g, 51 mmol) was stirred for two hours at 105° C. The mixture was cooled and quenched by the addition of ice water (100 mL). The mixture was extracted three times with dichloromethane, and the combined organic layers were concentrated to afford imidazo[1,2-a]pyridine-8-sulfonyl chloride (1.05 g, 48%) as a light yellow solid.

### Example 34—Synthesis of 5-Cyano-2-methoxypyridine-3-sulfonyl chloride

### Intermediate

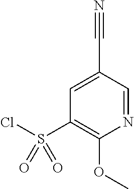

### Part I—Synthesis of 5-Bromo-6-methoxynicotinonitrile

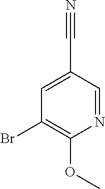

A solution of 5-bromo-6-chloropyridine-3-carbonitrile (4.00 g, 18.4 mmol), methanol (20 mL), and sodium methoxide (20 mg, 0.370 mmol) was stirred overnight at 80° C. The mixture was concentrated, and the residue was purified via MPLC eluting with 20% ethyl acetate in petroleum ether to afford 5-bromo-6-methoxynicotinonitrile (1.5 g, 38%) as an off-white solid.

### Part II—Synthesis of 5-(Benzylthio)-6-methoxynicotinonitrile

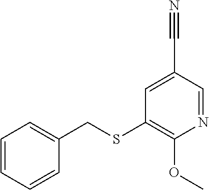 [SMILES: COc1ncc(C#N)cc1SCc1ccccc1]

A mixture of 5-bromo-6-methoxypyridine-3-carbonitrile (2.00 g, 9.39 mmol), toluene (30 mL), N,N-diisopropylethylamine (3.06 mL, 17.6 mmol), 4,5-bis(diphenylphosphino)-9,9-dimethylxanthene (538 mg, 0.93 mmol), tris(dibenzylideneacetone)dipalladium(0) (425 mg, 0.46 mmol), benzyl mercaptan (1.1 mL, 1.34 mmol) was stirred for two hours at 110° C. The mixture was concentrated, and the residue was purified via MPLC eluting with ethyl 16% acetate in petroleum ether to afford 5-(benzylthio)-6-methoxynicotinonitrile (1.5 g, 62%) as a yellow solid.

### Part III—Synthesis of 5-Cyano-2-methoxypyridine-3-sulfonyl chloride

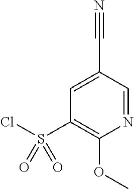

A mixture of 5-(benzylthio)-6-methoxynicotinonitrile (1.00 g, 3.90 mmol), acetic acid (15 mL), water (5 mL), and N-chlorosuccinimide (2.08 g, 0.016 mmol) was stirred for two hours at room temperature. The mixture was diluted with water and was extracted twice with ethyl acetate. The combined organic layers were dried (Na₂SO₄) and concentrated. The residue was purified via MPLC eluting with 20% ethyl acetate in petroleum ether to afford 5-cyano-2-methoxypyridine-3-sulfonyl chloride (900 mg, 99%) as a yellow solid.

### Example 35—Synthesis of 6-[3-[(5-chloro-2-methoxy-3-pyridyl)sulfonylamino]-2-fluoro-phenyl]-N-[3-(dimethylamino)propyl]-1H-pyrazolo[4,3-c]pyridine-3-carboxamide

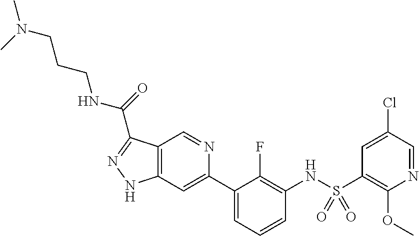

### Part I—Synthesis of 6-chloro-3-iodo-1H-pyrazolo[4,3-c]pyridine

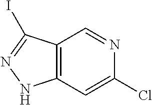 [SMILES: Clc1cc2[nH]nc(I)c2cn1]

A mixture of 6-chloro-1H-pyrazolo[4,3-c]pyridine (3.0 g, 19.5 mmol), DMF (100 mL), potassium hydroxide (3.29 g, 58.6 mmol) and iodine (8.9248 g, 35.163 mmol) was stirred at 50° C. overnight. The mixture was cooled, and quenched with aqueous sodium thiosulfate, diluted with water, and extracted three times with ethyl acetate. The combined organic layers were washed with water, brine, dried (MgSO₄) and concentrated. The residue was triturated with dichloromethane, filtered, and dried to afford 6-chloro-3-iodo-1H-pyrazolo[4,3-c]pyridine (2.06 g).

### Part II— Synthesis of methyl 6-chloro-1H-pyrazolo[4,3-c]pyridine-3-carboxylate

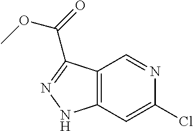

A mixture of 6-chloro-3-iodo-1H-pyrazolo[4,3-c]pyridine (470 mg, 1.7 mmol) in triethylamine (5.80 g, 58 mmol), methyl alcohol (1.10 g, 34 mmol), 4,5-bis(diphenylphosphino)-9,9-dimethylxanthene (40 mg, 0.07 mmol), and palladium acetate (8 mg, 0.04 mmol) was stirred at 50° C. under an atmosphere of carbon monoxide for three days. The mixture was concentrated and purified via MPLC eluting with a gradient of 0-100% ethyl acetate in hexanes to afford methyl 6-chloro-1H-pyrazolo[4,3-c]pyridine-3-carboxylate (230 mg, 64% yield).

### Part III—Synthesis of methyl 6-chloro-1-(2-trimethylsilylethoxymethyl)pyrazolo[4,3-c]pyridine-3-carboxylate

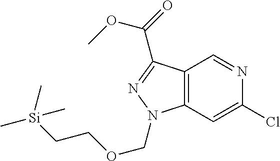 [SMILES: COC(=O)c1nn(COCC[Si](C)(C)C)c2cc(Cl)ncc12]

Sodium hydride, 60% in mineral oil (0.1 g, 2.9 mmol) was added portionwise to a stirred solution of methyl 6-chloro-1H-pyrazolo[4,3-c]pyridine-3-carboxylate (0.55 g, 2.6 mmol) in THE (20 mL) at 0° C. and stirred for an additional hour. 2-(Trimethylsilyl)ethoxymethyl chloride (460 mg, 2.7 mmol) was added and the mixture was stirred overnight at room temperature. The mixture was quenched with saturated ammonium chloride, and the mixture was extracted three times with ethyl acetate. The combined organic layers were washed with brine, dried (MgSO₄) and concentrated. The residue was purified by MPLC eluting with 0-30% ethyl acetate in hexanes to afford methyl 6-chloro-1-(2-trimethylsilylethoxymethyl)pyrazolo[4,3-c]pyridine-3-carboxylate (660 mg, 74% yield) as an off-white solid.

### Part IV—Synthesis of 5-chloro-N-[2-fluoro-3-(4,4,5,5-tetramethyl-1,3,2-dioxaborolan-2-yl)phenyl]-2-methoxy-pyridine-3-sulfonamide

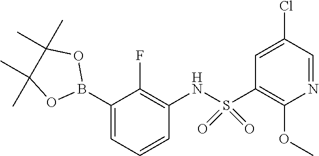

A solution of 2-fluoro-3-(4,4,5,5-tetramethyl-1,3,2-dioxaborolan-2-yl)aniline (580 mg, 2.4 mmol), 5-chloro-2-methoxy-pyridine-3-sulfonyl chloride (600 mg, 2.4 mmol), and pyridine (2.5 mL) was stirred at room temperature overnight. The mixture was concentrated, and the residue was partitioned between ethyl acetate and saturated aqueous sodium bicarbonate. The aqueous layer was extracted three times with ethyl acetate. The combined organic layers were washed with brine, dried (MgSO₄) and concentrated. The residue was purified by MPLC eluting with 0-30% ethyl acetate in hexanes to afford 5-chloro-N-[2-fluoro-3-(4,4,5,5-tetramethyl-1,3,2-dioxaborolan-2-yl)phenyl]-2-methoxy-pyridine-3-sulfonamide (910 mg, 84% yield) as an off-white solid.

### Part V—Synthesis of methyl 6-[3-[(5-chloro-2-methoxy-3-pyridyl)sulfonylamino]-2-fluoro-phenyl]-1-(2-trimethylsilylethoxymethyl)pyrazolo[4,3-c]pyridine-3-carboxylate

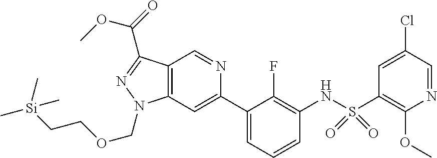

A mixture of methyl 6-chloro-1-(2-trimethylsilylethoxymethyl)pyrazolo[4,3-c]pyridine-3-carboxylate (170 mg, 0.49 mmol), 5-chloro-N-[2-fluoro-3-(4,4,5,5-tetramethyl-1,3,2-dioxaborolan-2-yl)phenyl]-2-methoxy-pyridine-3-sulfonamide (220 mg, 0.49 mmol), potassium phosphate (260 mg, 1.2 mmol), 1,4-dioxane (5 mL), water (1 mL), and [1,3-bis(2,6-diisopropylphenyl)imidazol-2-ylidene](3-chloropyridyl)palladium(II) dichloride (16 mg, 0.024 mmol) was stirred at 65° C. for three hours. The mixture was quenched with saturated sodium bicarbonate and was extracted three times with ethyl acetate. The combined organic layers were washed with brine, dried (MgSO₄), and concentrated. The residue was purified by MPLC eluting with 0-40% ethyl acetate in hexanes to afford methyl 6-[3-[(5-chloro-2-methoxy-3-pyridyl)sulfonylamino]-2-fluoro-phenyl]-1-(2-trimethylsilylethoxymethyl)pyrazolo[4,3-c]pyridine-3-carboxylate (123 mg, 41% yield).

### Part VI—Synthesis of methyl 6-[3-[(5-chloro-2-methoxy-3-pyridyl)sulfonylamino]-2-fluoro-phenyl]-1H-pyrazolo[4,3-c]pyridine-3-carboxylate

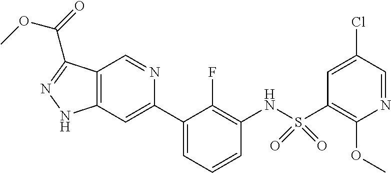

A mixture of methyl 6-[3-[(5-chloro-2-methoxy-3-pyridyl)sulfonylamino]-2-fluoro-phenyl]-1-(2-trimethylsilylethoxymethyl)pyrazolo[4,3-c]pyridine-3-carboxylate (140 mg, 0.30 mmol), dichloromethane (5 mL), and trifluoroacetic acid (1.8 g, 16 mmol) was stirred at room temperature for three hours. The mixture was concentrated, and the residue was re-dissolved in MeOH containing 10% NH₄OH. The mixture was stirred at room temperature for two hours, concentrate, and the residue was purified by MPLC eluting with 0-5% methanol in dichloromethane to afford methyl 6-[3-[(5-chloro-2-methoxy-3-pyridyl)sulfonylamino]-2-fluoro-phenyl]-1H-pyrazolo[4,3-c]pyridine-3-carboxylate (84 mg, 74% yield) as a white solid.

### Part VII—Synthesis of 6-[3-[(5-chloro-2-methoxy-3-pyridyl)sulfonylamino]-2-fluoro-phenyl]-N-[3-(dimethylamino)propyl]-1H-pyrazolo[4,3-c]pyridine-3-carboxamide

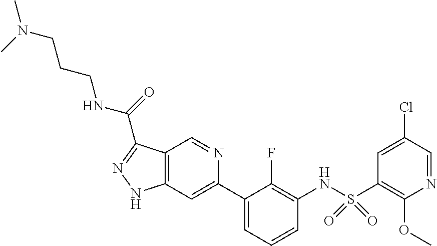

A mixture of methyl 6-[3-[(5-chloro-2-methoxy-3-pyridyl)sulfonylamino]-2-fluoro-phenyl]-1H-pyrazolo[4,3-c]pyridine-3-carboxylate (20 mg, 0.04 mmol) and N,N-dimethyl-1,3-propanediamine (40 mg, 0.39 mmol) was stirred at 90° C. overnight. The mixture was concentrated, and the residue purified by MPLC eluting with 0-20% methanol in dichloromethane to afford 6-[3-[(5-chloro-2-methoxy-3-pyridyl)sulfonylamino]-2-fluoro-phenyl]-N-[3-(dimethylamino)propyl]-1H-pyrazolo[4,3-c]pyridine-3-carboxamide (8 mg, 39% yield) as a white solid. ¹H NMR (400 MHz, CDCl₃): δ 9.63 (m, 1H), 8.32 (br s, 1H), 8.19 (m, 1H), 8.07 (m, 1H), 7.64-7.76 (m, 2H), 7.52 (br t, J=7.1 Hz, 1H), 7.13 (m, 1H), 4.04 (s, 3H), 3.55-3.72 (m, 2H), 2.98 (br s, 2H), 2.66 (s, 6H), 2.08-2.22 (m, 2H). (ES, m/z): (M+H)⁺ 562.29, 564.27.

### Example 36—Synthesis of 5-chloro-N-[2,5-difluoro-3-[7-fluoro-3-(1H-imidazol-2-yl)-1H-pyrazolo[4,3-c]pyridin-6-yl]phenyl]-2-methoxy-pyridine-3-sulfonamide

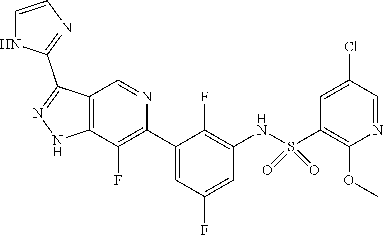 [SMILES: COc1ncc(Cl)cc1S(=O)(=O)Nc1cc(F)cc(-c2ncc3c(-c4ncc[nH]4)n[nH]c3c2F)c1F]

### Part I—Synthesis of N-methoxy-N-methyl-1-(2-trimethylsilylethoxymethyl)imidazole-2-carboxamide

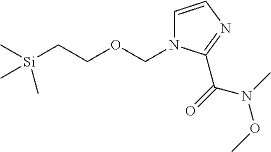

To a solution of ethyl 1-(2-trimethylsilylethoxymethyl)imidazole-2-carboxylate (10.7 g, 39.7 mmol) in 1,4-dioxane (240 mL) and water (80 mL) was added lithium hydroxide (1.05 g, 43.7 mmol). The mixture was stirred at 50° C. for four hours then concentrated. The residue was suspended in toluene and concentrated again. A mixture of the residual lithium salt, N,N-diisopropylethylamine (10.3 g, 79.4 mmol), 2-(1H-benzotriazole-1-yl)-1,1,3,3-tetramethylaminium tetrafluoroborate (17.2 g, 53.6 mmol), DMF (70 mL), dichloromethane (70 mL) and N,O-dimethylhydroxylamine hydrochloride (4.3 g, 43.7 mmol) was stirred at room temperature overnight. Water (200 mL) was added, and the product was extracted with EtOAc (3×100 mL). The combined organics were washed with water (1×60 mL), brine (3×60 mL), dried (MgSO₄), and concentrated. The residue was purified by MPLC eluting with 0-40% ethyl acetate in hexanes to afford N-methoxy-N-methyl-1-(2-trimethylsilylethoxymethyl)imidazole-2-carboxamide (3.27 g, 29% yield) as a light brown clear liquid.

### Part II—Synthesis of (4,6-dichloro-5-fluoro-3-pyridyl)-[1-(2-trimethylsilylethoxymethyl)-imidazol-2-yl]methanone

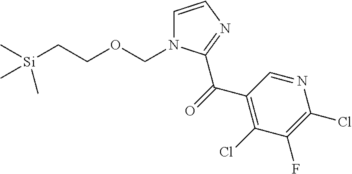 [SMILES: C[Si](C)(C)CCOCn1ccnc1C(=O)c1cnc(Cl)c(F)c1Cl]

A 2M solution of lithium diisopropylamide in THE (7.25 mL, 14.5 mmol) was added dropwise to a solution of 2,4-dichloro-3-fluoro-pyridine (2.40 g, 14.5 mmol) in THE (25 mL) at −78° C. The mixture was stirred for an additional hour at −78° C. A solution of N-methoxy-N-methyl-1-(2-trimethylsilylethoxymethyl)imidazole-2-carboxamide (3.4 g, 12 mmol) in THE (11 mL) was added dropwise over 5 minutes, then the mixture was stirred at −78° C. for an additional hour, and at room temperature overnight. Saturated aqueous ammonium chloride was added, and the mixture was extracted three times with ethyl acetate. The combined organic layers were washed with brine, dried (MgSO₄), and concentrated. The residue was purified by MPLC eluting with 0-30% ethyl acetate in hexanes to afford (4,6-dichloro-5-fluoro-3-pyridyl)-[1-(2-trimethylsilylethoxymethyl)imidazol-2-yl]methanone (2.16 g, 46%) as a light yellow clear liquid.

### Part III—2-[[2-(6-chloro-7-fluoro-1H-pyrazolo[4,3-c]pyridin-3-yl)imidazol-1-yl]methoxy]-ethyl-trimethyl-silane

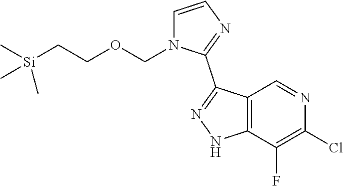 [SMILES: C[Si](C)(C)CCOCn1ccnc1-c1n[nH]c2c(F)c(Cl)ncc12]

A stirred solution of (4,6-dichloro-5-fluoro-3-pyridyl)-[1-(2-trimethylsilylethoxymethyl)imidazol-2-yl]methanone (1.4 g, 3.6 mmol), tert-butyl carbazate (940 mg, 7.143 mmol), acetic acid (430 mg, 7.1 mmol) and molecular sieves in methanol (12 mL) was heated at reflux for three days. The suspension was filtered through celite, the filter cake washed with 10% methanol in dichloromethane. The filtrate was diluted with saturated solution of sodium bicarbonate, and the mixture was extracted with dichloromethane (5×40 mL). The combined organic layers were dried (MgSO₄) and concentrated. The residue was purified by MPLC eluting with 0-30% ethyl acetate in hexanes to afford 2-[[2-(6-chloro-7-fluoro-1H-pyrazolo[4,3-c]pyridin-3-yl)imidazol-1-yl]methoxy]ethyl-trimethyl-silane (900 mg, 68% yield) as an off-white solid.

### Part IV—2-[[6-chloro-7-fluoro-3-[1-(2-trimethylsilylethoxymethyl)imidazol-2-yl]pyrazolo[4,3-c]pyridin-1-yl]methoxy]ethyl-trimethyl-silane

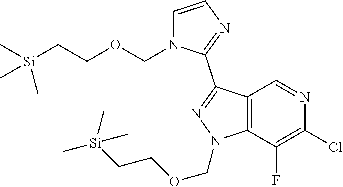 [SMILES: C[Si](C)(C)CCOCn1ccnc1-c1nn(COCC[Si](C)(C)C)c2c(F)c(Cl)ncc12]

Sodium hydride (60% in mineral oil, 100 mg, 2.7 mmol) was added portionwise to a stirred solution of 2-[[2-(6-chloro-7-fluoro-1H-pyrazolo[4,3-c]pyridin-3-yl)imidazol-1-yl]methoxy]ethyl-trimethyl-silane (900 mg, 2.5 mmol) in THE (20 mL) at 0° C. and stirred for an additional hour at 0° C. 2-(Trimethylsilyl)ethoxymethyl chloride (430 mg, 2.6 mmol) was added dropwise, and the mixture was stirred overnight at room temperature. The mixture was quenched with saturated aqueous ammonium chloride, and the mixture was extracted three times with dichloromethane. The combined organic layers were dried (MgSO₄) and concentrated. The residue was purified by MPLC eluting with 0-20% ethyl acetate in hexanes to afford 2-[[6-chloro-7-fluoro-3-[1-(2-trimethylsilylethoxymethyl)imidazol-2-yl]pyrazolo[4,3-c]pyridin-1-yl]methoxy]ethyl-trimethyl-silane (743 mg, 61% yield) as an off-white solid.

### Part V—5-chloro-N-[2,5-difluoro-3-(4,4,5,5-tetramethyl-1,3,2-dioxaborolan-2-yl)phenyl]-2-methoxy-pyridine-3-sulfonamide

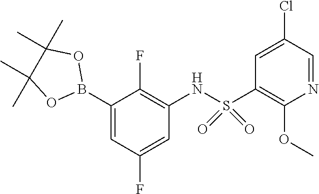

A solution of 2,5-difluoro-3-(4,4,5,5-tetramethyl-1,3,2-dioxaborolan-2-yl)aniline (370 mg, 1.46 mmol) and 5-chloro-2-methoxy-pyridine-3-sulfonyl chloride (390 mg, 1.6 mmol) in pyridine (2.5 mL) was stirred at room temperature overnight. The mixture was concentrated, and the residue was partitioned between ethyl acetate and saturated aqueous sodium bicarbonate. The aqueous layer was extracted three times with ethyl acetate, and the combined organic layers were washed with brine, dried (MgSO₄) and concentrated. The residue was purified by MPLC eluting with 0-30% ethyl acetate in hexanes to afford 5-chloro-N-[2,5-difluoro-3-(4,4,5,5-tetramethyl-1,3,2-dioxaborolan-2-yl)phenyl]-2-methoxy-pyridine-3-sulfonamide (382 mg, 57% yield) as a white solid.

### Part VI— 5-chloro-N-[2,5-difluoro-3-[7-fluoro-1-(2-trimethylsilylethoxymethyl)-3-[1-(2-trimethylsilylethoxymethyl)imidazol-2-yl]pyrazolo[4,3-c]pyridin-6-yl]phenyl]-2-methoxy-pyridine-3-sulfonamide

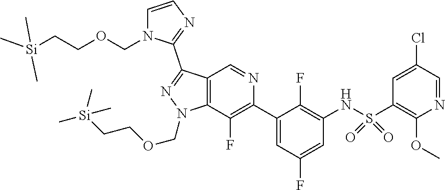

A degassed mixture of 2-[[6-chloro-7-fluoro-3-[1-(2-trimethylsilylethoxymethyl)imidazol-2-yl]pyrazolo[4,3-c]pyridin-1-yl]methoxy]ethyl-trimethyl-silane (140 mg, 0.27 mmol), 5-chloro-N-[2,5-difluoro-3-(4,4,5,5-tetramethyl-1,3,2-dioxaborolan-2-yl)phenyl]-2-methoxy-pyridine-3-sulfonamide (140 mg, 0.3 mmol), potassium phosphate (140 mg, 0.7 mmol), 1,4-dioxane (3 mL), water (0.6 mL), and [1,3-bis(2,6-diisopropylphenyl)imidazol-2-ylidene](3-chloropyridyl)palladium(II) dichloride (9.2 mg, 0.014 mmol) was stirred at 90° C. for three days. The mixture was partitioned between ethyl acetate and water, and the aqueous phase extracted three times with ethyl acetate. The combined organic layers were washed with brine, dried (MgSO₄) and concentrated. The residue was purified by MPLC eluting with 0-30% ethyl acetate in hexanes to afford 5-chloro-N-[2,5-difluoro-3-[7-fluoro-1-(2-trimethylsilylethoxymethyl)-3-[1-(2-trimethylsilylethoxymethyl)imidazol-2-yl]pyrazolo[4,3-c]pyridin-6-yl]phenyl]-2-methoxy-pyridine-3-sulfonamide (51 mg, 24% yield) as a light yellow liquid.

### Part VII—5-chloro-N-[2,5-difluoro-3-[7-fluoro-3-(1H-imidazol-2-yl)-1H-pyrazolo[4,3-c]pyridin-6-yl]phenyl]-2-methoxy-pyridine-3-sulfonamide

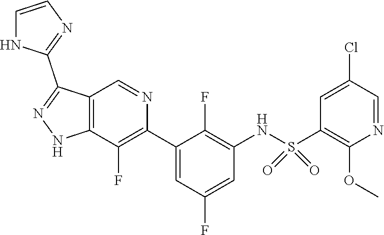 [SMILES: COc1ncc(Cl)cc1S(=O)(=O)Nc1cc(F)cc(-c2ncc3c(-c4ncc[nH]4)n[nH]c3c2F)c1F]

A solution of 5-chloro-N-[2,5-difluoro-3-[7-fluoro-1-(2-trimethylsilylethoxymethyl)-3-[1-(2-trimethylsilylethoxymethyl)imidazol-2-yl]pyrazolo[4,3-c]pyridin-6-yl]phenyl]-2-methoxy-pyridine-3-sulfonamide (50 mg, 0.06 mmol), dichloromethane (2 mL) and trifluoroacetic acid (1.5 g, 13 mmol) was stirred at room temperature overnight. The mixture was concentrated, and the residue was diluted with methanol containing 10% NH₄OH. The solution was stirred at room temperature for two hours then concentrated. The residue was purified by MPLC eluting with 0-15% methanol in dichloromethane to afford 5-chloro-N-[2,5-difluoro-3-[7-fluoro-3-(1H-imidazol-2-yl)-1H-pyrazolo[4,3-c]pyridin-6-yl]phenyl]-2-methoxy-pyridine-3-sulfonamide (21.8 mg, 62% yield) as a white solid. ¹H NMR (400 MHz, CD₃OD): δ 9.42 (m, 1H), 8.32 (br d, J=1.4 Hz, 1H), 8.16 (br s, 1H), 7.42 (br d, J=2.7 Hz, 1H), 7.26 (s, 2H), 7.17 (m, 1H), 3.97 (s, 3H). (ES, m/z): (M+H)⁺ 536.14, 538.14.

### Example 37—Preparation of Additional (Aza)Indazolyl-Aryl Sulfonamide and Related Compounds

Compounds in Table 5 were prepared based on experimental procedures described in Examples 1-36 and in the Detailed Description. Additional physical characterization data (e.g., ¹H NMR data) for exemplary compounds are provided in Table 6.

TABLE 5

| Compd | | | Observed |
|---|---|---|---|
| No. | Structure | Name | m/z |
| 3A | 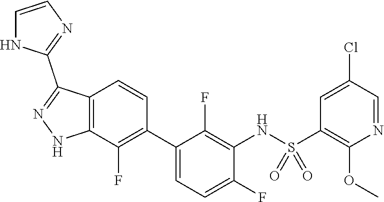 [SMILES: COc1ncc(Cl)cc1S(=O)(=O)Nc1c(F)ccc(-c2ccc3c(-c4ncc[nH]4)n[nH]c3c2F)c1F] | 5-chloro-N-(2,6-difluoro-3- (7-fluoro-3-(1H-imidazol- 2-yl)-1H-indazol-6- yl)phenyl)-2- methoxypyridine-3- sulfonamide | 535 (M + H)⁺ |
| 3B | 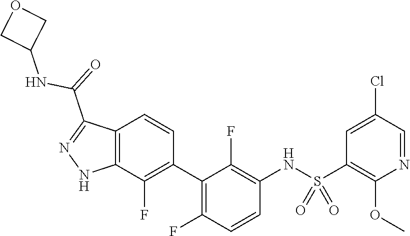 [SMILES: COc1ncc(Cl)cc1S(=O)(=O)Nc1ccc(F)c(-c2ccc3c(C(=O)NC4COC4)n[nH]c3c2F)c1F] | 6-(3-((5-chloro-2-methoxy- pyridine)-3-sulfonamido)- 2,6-difluorophenyl)-7- fluoro-N-(oxetan-3-yl)-1H- indazole-3-carboxamide | 568 (M + H)⁺ |
| 3C | 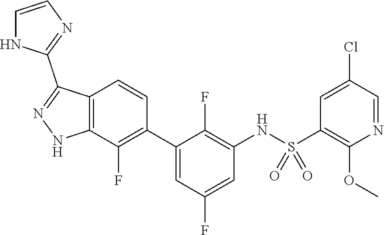 [SMILES: COc1ncc(Cl)cc1S(=O)(=O)Nc1cc(F)cc(-c2ccc3c(-c4ncc[nH]4)n[nH]c3c2F)c1F] | 5-chloro-N-(2,5-difluoro-3- (7-fluoro-3-(1H-imidazol- 2-yl)-1H-indazol-6- yl)phenyl)-2- methoxypyridine-3- sulfonamide | 535 (M + H)⁺ |
| 3D | 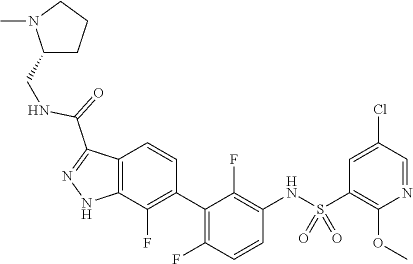 | 6-(3-((5-chloro-2-methoxy- pyridine)-3-sulfonamido)- 2,6-difluorophenyl)-7- fluoro-N-(((R)-1- methylpyrrolidin-2- yl)methyl)-1H-indazole-3- carboxamide | 609 (M + H)⁺ |
| 3E | 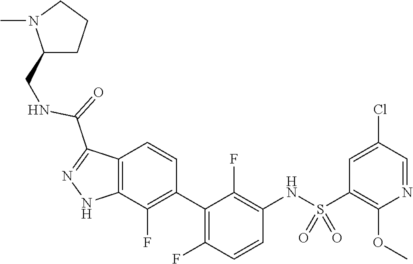 | 6-(3-((5-chloro-2-methoxy- pyridine)-3-sulfonamido)- 2,6-difluorophenyl)-7- fluoro-N-(((S)-1- methylpyrrolidin-2- yl)methyl)-1H-indazole-3- carboxamide | 609 (M + H)⁺ |
| 3F | 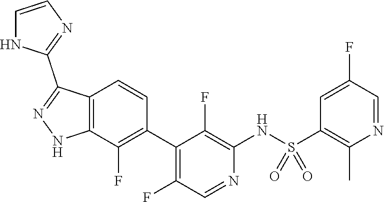 [SMILES: Cc1ncc(F)cc1S(=O)(=O)Nc1ncc(F)c(-c2ccc3c(-c4ncc[nH]4)n[nH]c3c2F)c1F] | N-(3,5-difluoro-4-(7- fluoro-3-(1H-imidazol-2- yl)-1H-indazol-6- yl)pyridin-2-yl)-5-fluoro-2- methylpyridine-3- sulfonamide | 504 (M + H)⁺ |
| 3G | 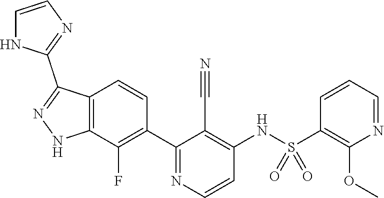 [SMILES: COc1ncccc1S(=O)(=O)Nc1ccnc(-c2ccc3c(-c4ncc[nH]4)n[nH]c3c2F)c1C#N] | N-(3-cyano-2-(7-fluoro-3- (1H-imidazol-2-yl)-1H- indazol-6-yl)pyridin-4-yl)- 2-methoxypyridine-3- sulfonamide | 491 (M + H)⁺ |
| 3H | 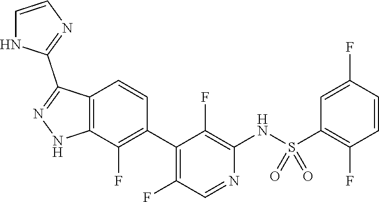 [SMILES: O=S(=O)(Nc1ncc(F)c(-c2ccc3c(-c4ncc[nH]4)n[nH]c3c2F)c1F)c1cc(F)ccc1F] | N-(3,5-difIuoro-4-(7- fluoro-3-(1H-imidazol-2- yl)-1H-indazol-6- yl)pyridin-2-yl)-2,5- difluorobenzene- sulfonamide | 507 (M + H)⁺ |
| 3I | 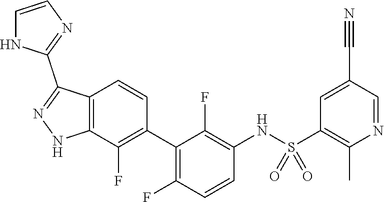 [SMILES: Cc1ncc(C#N)cc1S(=O)(=O)Nc1ccc(F)c(-c2ccc3c(-c4ncc[nH]4)n[nH]c3c2F)c1F] | 5-cyano-N-(2,4-difluoro-3- (7-fluoro-3-(1H-imidazol- 2-yl)-1H-indazol-6- yl)phenyl)-2- methylpyridine-3- sulfonamide | 510 (M + H)⁺ |
| 3J | 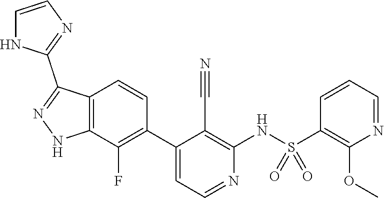 [SMILES: COc1ncccc1S(=O)(=O)Nc1nccc(-c2ccc3c(-c4ncc[nH]4)n[nH]c3c2F)c1C#N] | N-(3-cyano-4-(7-fluoro-3- (1H-imidazol-2-yl)-1H- indazol-6-yl)pyridin-2-yl)- 2-methoxypyridine-3- sulfonamide | 491 (M + H)⁺ |
| 3K | 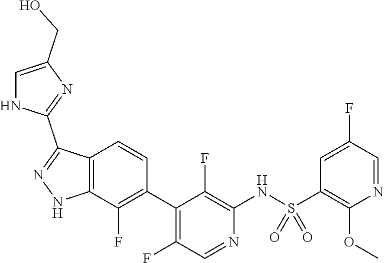 [SMILES: COc1ncc(F)cc1S(=O)(=O)Nc1ncc(F)c(-c2ccc3c(-c4nc(CO)c[nH]4)n[nH]c3c2F)c1F] | N-(3,5-difluoro-4-(7- fluoro-3-(4- (hydroxymethyl)-1H- imidazol-2-yl)-1H-indazol- 6-yl)pyridin-2-yl)-5-fluoro- 2-methoxypyridine-3- sulfonamide | 550 (M + H)⁺ |
| 3L | 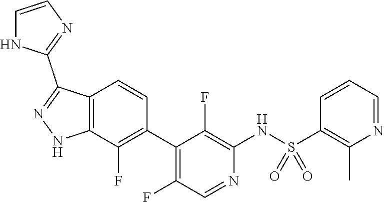 [SMILES: Cc1ncccc1S(=O)(=O)Nc1ncc(F)c(-c2ccc3c(-c4ncc[nH]4)n[nH]c3c2F)c1F] | N-(3,5-difluoro-4-(7- fluoro-3-(1H-imidazol-2- yl)-1H-indazol-6- yl)pyridin-2-yl)-2- methylpyridine-3- sulfonamide | 486 (M + H)⁺ |
| 3M | 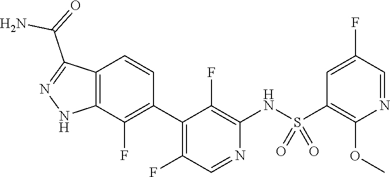 [SMILES: COc1ncc(F)cc1S(=O)(=O)Nc1ncc(F)c(-c2ccc3c(C(N)=O)n[nH]c3c2F)c1F] | 6-(3,5-difluoro-2-((5- fluoro-2-methoxypyridine)- 3-sulfonamido)pyridin-4- yl)-7-fluoro-1H-indazole-3- carboxamide | 497 (M + H)⁺ |
| 3N | 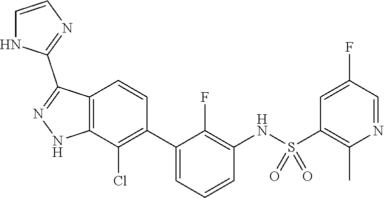 [SMILES: Cc1ncc(F)cc1S(=O)(=O)Nc1cccc(-c2ccc3c(-c4ncc[nH]4)n[nH]c3c2Cl)c1F] | N-(3-(7-chloro-3-(1H- imidazol-2-yl)-1H-indazol- 6-yl)-2-fluorophenyl)-5- fluoro-2-methylpyridine-3- sulfonamide | 501 (M + H)⁺ |
| 3O | 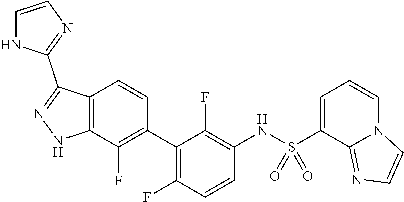 [SMILES: O=S(=O)(Nc1ccc(F)c(-c2ccc3c(-c4ncc[nH]4)n[nH]c3c2F)c1F)c1cccn2ccnc12] | N-(2,4-difIuoro-3-(7- fluoro-3-(1H-imidazol-2- yl)-1H-indazol-6- yl)phenyl)-imidazo[1,2- a]pyridine-8-sulfonamide | 510 (M + H)⁺ |
| 3P | 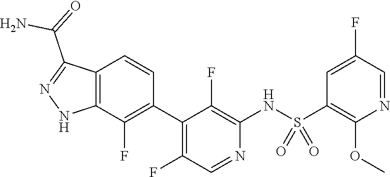 [SMILES: COc1ncc(F)cc1S(=O)(=O)Nc1ncc(F)c(-c2ccc3c(C(N)=O)n[nH]c3c2F)c1F] | 6-(3,5-difluoro-2-((5- fluoro-2-methoxypyridine)- 3-sulfonamido)pyridin-4- yl)-1H-indazole-3- carboxamide | 479 (M + H)⁺ |
| 3Q | 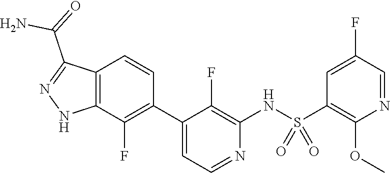 [SMILES: COc1ncc(F)cc1S(=O)(=O)Nc1nccc(-c2ccc3c(C(N)=O)n[nH]c3c2F)c1F] | 6-(3-fluoro-2-((5-fluoro-2- methoxypyridine)-3- sulfonamido)pyridin-4-yl)- 7-fluoro-1H-indazole-3- carboxamide | 479 (M + H)⁺ |
| 3R | 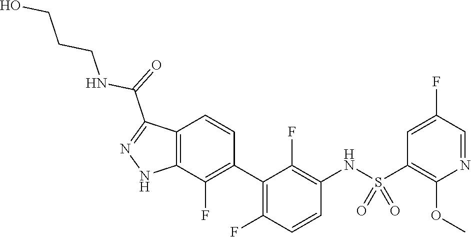 [SMILES: COc1ncc(F)cc1S(=O)(=O)Nc1ccc(F)c(-c2ccc3c(C(=O)NCCCO)n[nH]c3c2F)c1F] | 6-(2,6-difluoro-3-((5- fluoro-2-methoxypyridine)- 3-sulfonamido)phenyl)-7- fluoro-N-(3- hydroxypropyl)-1H- indazole-3-carboxamide | 554 (M + H)⁺ |
| 3S | 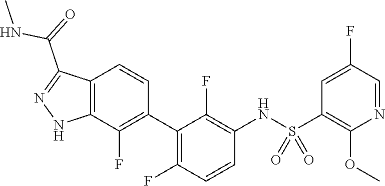 | 6-(2,6-difluoro-3-((5- fluoro-2-methoxypyridine)- 3-sulfonamido)phenyl)-7- fluoro-N-methyl-1H- indazole-3-carboxamide | 510 (M + H)⁺ |
| 3T | 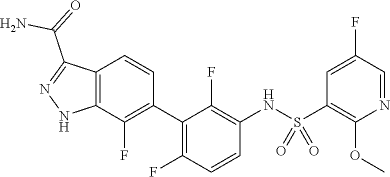 [SMILES: COc1ncc(F)cc1S(=O)(=O)Nc1ccc(F)c(-c2ccc3c(C(N)=O)n[nH]c3c2F)c1F] | 6-(2,6-difluoro-3-((5- fluoro-2-methoxypyridine)- 3-sulfonamido)phenyl)-7- fluoro-1H-indazole-3- carboxamide | 496 (M + H)⁺ |
| 3U | 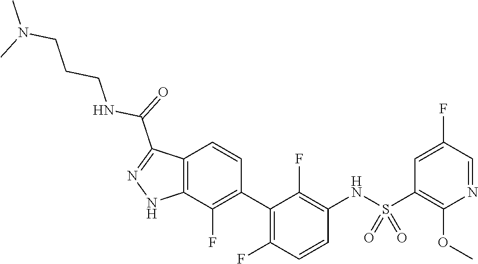 | 6-(2,6-difluoro-3-((5- fluoro-2-methoxypyridine)- 3-sulfonamido)phenyl)-N- (3-(dimethylamino)propyl)- 7-fluoro-1H-indazole-3- carboxamide | 581 (M + H)⁺ |
| 3V | 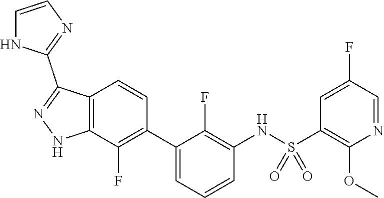 [SMILES: COc1ncc(F)cc1S(=O)(=O)Nc1cccc(-c2ccc3c(-c4ncc[nH]4)n[nH]c3c2F)c1F] | N-(2-fluoro-3-(7-fluoro-3- (1H-imidazol-2-yl)-1H- indazol-6-yl)phenyl)-5- fluoro-2-methoxypyridine- 3-sulfonamide | 501 (M + H)⁺ |
| 3W | 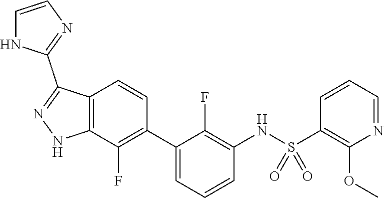 [SMILES: COc1ncccc1S(=O)(=O)Nc1cccc(-c2ccc3c(-c4ncc[nH]4)n[nH]c3c2F)c1F] | N-(2-fluoro-3-(7-fluoro-3- (1H-imidazol-2-yl)-1H- indazol-6-yl)phenyl)-2- methoxypyridine-3- sulfonamide | 483 (M + H)⁺ |
| 3X | 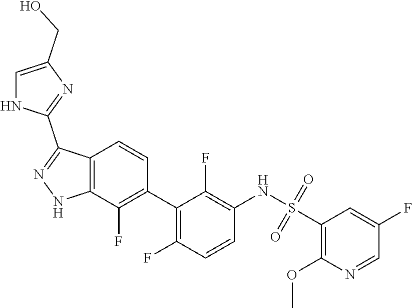 [SMILES: COc1ncc(F)cc1S(=O)(=O)Nc1ccc(F)c(-c2ccc3c(-c4nc(CO)c[nH]4)n[nH]c3c2F)c1F] | N-(2,4-difluoro-3-(7- fluoro-3-(4- (hydroxymethyl)-1H- imidazol-2-yl)-1H-indazol- 6-yl)phenl)-5-fluoro-2- methoxypyridine-3- sulfonamide | 549 (M + H)⁺ |
| 3Y | 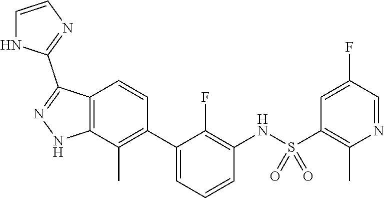 [SMILES: Cc1ncc(F)cc1S(=O)(=O)Nc1cccc(-c2ccc3c(-c4ncc[nH]4)n[nH]c3c2C)c1F] | N-(3-(3-(1H-imidazol-2- yl)-7-methyl-1H-indazol-6- yl)-2-fluorophenyl)-5- fluoro-2-methylpyridine-3- sulfonamide | 481 (M + H)⁺ |
| 3Z | 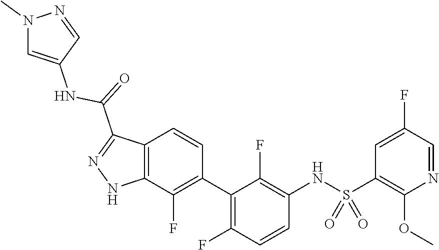 | 6-(2,6-difluoro-3-((5- fluoro-2-methoxypyridine)- 3-sulfonamido)phenyl)-7- fluoro-1H-(1-methyl-1H- pyrazol-4-yl)-1H-indazole- 3-carboxamide | 576 (M + H)⁺ |
| 3AA | 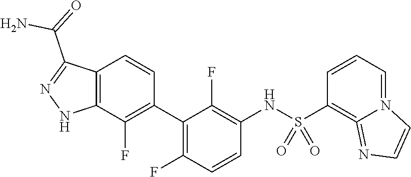 | 6-(2,6-difluoro-3-(imidazo- [1,2-a]pyridine-8-sulfon- amido)phenyl)-7-fluoro- 1H-indazole-3- carboxamide | 487 (M + H)⁺ |
| 3AB | 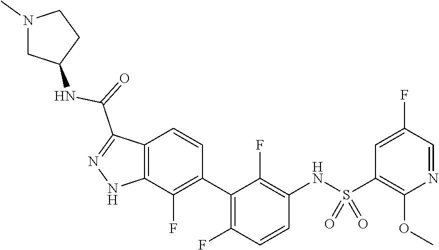 | 6-(3-((5-fluoro-2-methoxy- pyridine)-3-sulfonamido)- 2,6-difluorophenyl)-7- fluoro-N-((R)-1- methylpyrrolidin-3-yl)-1H- indazole-3-carboxamide | 579 (M + H)⁺ |
| 3AC | 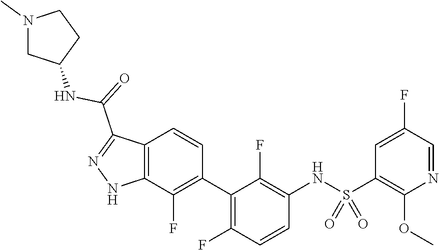 | 6-(3-((5-fluoro-2-methoxy- pyridine)-3-sulfonamido)- 2,6-difluorophenyl)-7- fluoro-N-((S)-1- methylpyrrolidin-3-yl)-1H- indazole-3-carboxamide | 579 (M + H)⁺ |
| 3AD | 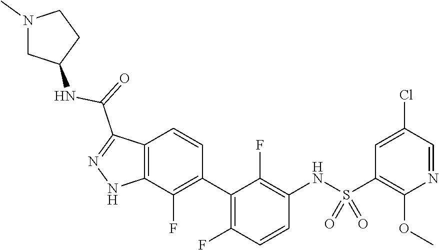 | 6-(3-((5-chloro-2-methoxy- pyridine)-3-sulfonamido)- 2,6-difluorophenyl)-7- fluoro-N-((R)-1- methylpyrrolidin-3-yl)-1H- indazole-3-carboxamide | 595 (M + H)⁺ |
| 3AF | 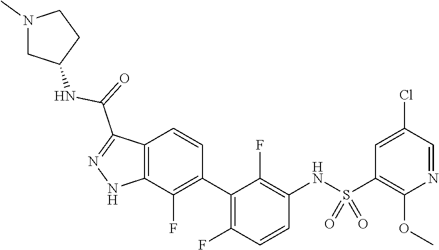 | 6-(3-((5-chloro-2-methoxy- pyridine)-3-sulfonamido)- 2,6-difluorophenyl)-7- fluoro-N-((S)-1- methylpyrrolidin-3-yl)-1H- indazole-3-carboxamide | 595 (M + H)⁺ |
| 3AG | 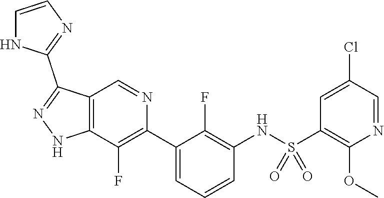 [SMILES: COc1ncc(Cl)cc1S(=O)(=O)Nc1cccc(-c2ncc3c(-c4ncc[nH]4)n[nH]c3c2F)c1F] | 5-chloro-N-(2-fluoro-3-(7- fluoro-3-(1H-imidazol-2- yl)-1H-pyrazolo[4,3- c]pyridin-6-yl)phenyl)-2- methoxy-pyridine-3- sulfonamide | 518 (M + H)⁺ |
| 3AH | 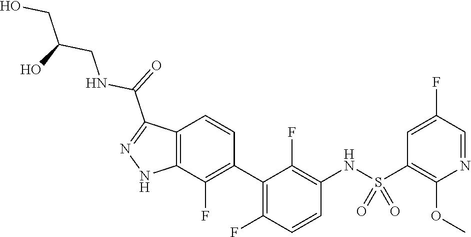 [SMILES: COc1ncc(F)cc1S(=O)(=O)Nc1ccc(F)c(-c2ccc3c(C(=O)NC[C@@H](O)CO)n[nH]c3c2F)c1F] | 6-(2,6-difluoro-3-((5- fluoro-2-methoxypyridine)- 3-sulfonamido)phenyl)-N- ((R)-2,3-dihydroxypropyl)- 7-fluoro-1H-indazole-3- carboxamide | 570 (M + H)⁺ |
| 3AI | 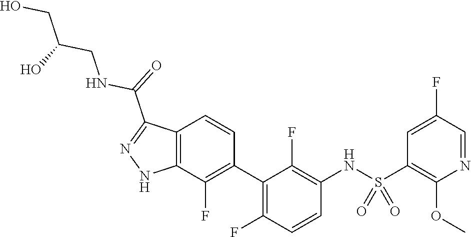 [SMILES: COc1ncc(F)cc1S(=O)(=O)Nc1ccc(F)c(-c2ccc3c(C(=O)NC[C@H](O)CO)n[nH]c3c2F)c1F] | 6-(2,6-difluoro-3-((5- fluoro-2-methoxypyridine)- 3-sulfonamido)phenyl)-N- ((R)-2,3-dihydroxypropyl)- 7-fluoro-1H-indazole-3- carboxamide | 570 (M + H)⁺ |
| 3AJ | 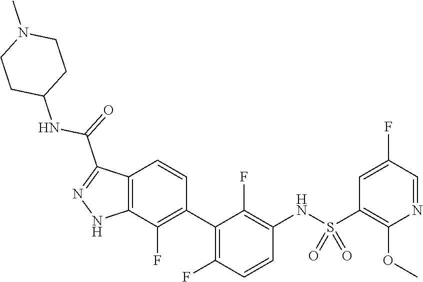 [SMILES: COc1ncc(F)cc1S(=O)(=O)Nc1ccc(F)c(-c2ccc3c(C(=O)NC4CCN(C)CC4)n[nH]c3c2F)c1F] | 6-(2,6-difluoro-3-((5- fluoro-2-methoxypyridine)- 3-sulfonamido)phenyl)-7- fluoro-N-(1- methylpiperidin-4-yl)-1H- indazole-3-carboxamide | 593 (M + H)⁺ |
| 3AK | 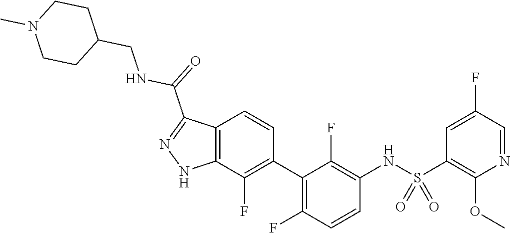 | 6-(2,6-difluoro-3-((5- fluoro-2-methoxypyridine)- 3-sulfonamido)phenyl)-7- fluoro-N-((1- methylpiperidin-4- yl)methyl)-1H-indazole-3- carboxamide | 607 (M + H)⁺ |
| 3AL | 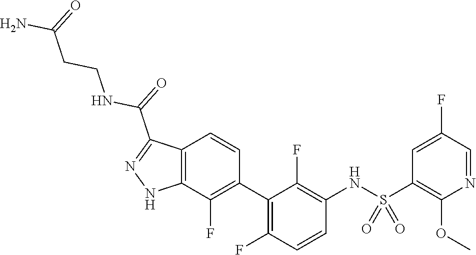 [SMILES: COc1ncc(F)cc1S(=O)(=O)Nc1ccc(F)c(-c2ccc3c(C(=O)NCCC(N)=O)n[nH]c3c2F)c1F] | N-(3-amino-3-oxopropyl)- 6-(2,6-difluoro-3-((5- fluoro-2-methoxypyridine)- 3-sulfonamido)phenyl)-7- fluoro-1H-indazole-3- carboxamide | 567 (M + H)⁺ |
| 3AM | 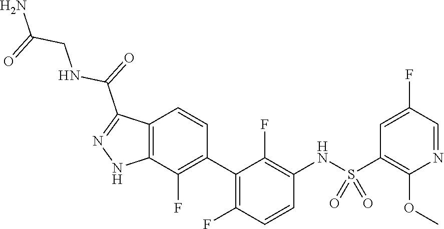 [SMILES: COc1ncc(F)cc1S(=O)(=O)Nc1ccc(F)c(-c2ccc3c(C(=O)NCC(N)=O)n[nH]c3c2F)c1F] | N-(2-amino-2-oxoethyl)-6- (2,6-difluoro-3-((5-fluoro- 2-methoxypyridine)-3- sulfon-amido)phenyl)-7- fluoro-1H-indazole-3- carboxamide | 553 (M + H)⁺ |
| 3AN | 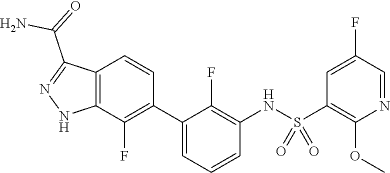 [SMILES: COc1ncc(F)cc1S(=O)(=O)Nc1cccc(-c2ccc3c(C(N)=O)n[nH]c3c2F)c1F] | 7-fluoro-6-(2-fluoro-3-((5- fluoro-2-methoxypyridine)- 3-sulfonamido)phenyl)-1H- indazole-3-carboxamide | 478 (M + H)⁺ |
| 3AO | 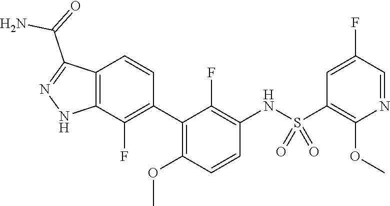 [SMILES: COc1ccc(NS(=O)(=O)c2cc(F)cnc2OC)c(F)c1-c1ccc2c(C(N)=O)n[nH]c2c1F] | 7-fluoro-6-(2-fluoro-3-((5- fluoro-2-methoxypyridine)- 3-sulfonamido)-6-methoxy- phenyl)-1H-indazole-3- carboxamide | 508 (M + H)⁺ |
| 3AP | 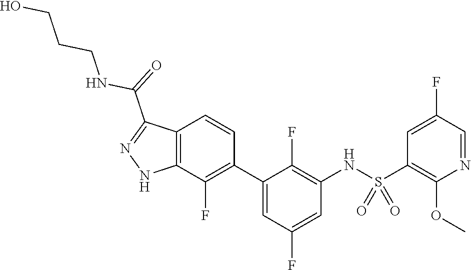 [SMILES: COc1ncc(F)cc1S(=O)(=O)Nc1cc(F)cc(-c2ccc3c(C(=O)NCCCO)n[nH]c3c2F)c1F] | 6-(2,5-difluoro-3-((5- fluoro-2-methoxypyridine)- 3-sulfon-amido)phenyl)-7- fluoro-N-(3- hydroxypropyl)-1H- indazole-3-carboxamide | 554 (M + H)⁺ |
| 3AQ | 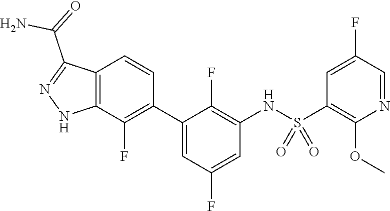 [SMILES: COc1ncc(F)cc1S(=O)(=O)Nc1cc(F)cc(-c2ccc3c(C(N)=O)n[nH]c3c2F)c1F] | 6-(2,5-difluoro-3-((5- fluoro-2-methoxypyridine)- 3-sulfon-amido)phenyl)-7- fluoro-1H-indazole-3- carboxamide | 496 (M + H)⁺ |
| 3AR | 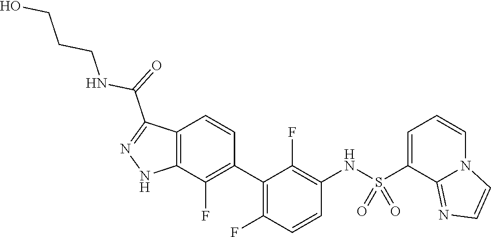 [SMILES: O=C(NCCCO)c1n[nH]c2c(F)c(-c3c(F)ccc(NS(=O)(=O)c4cccn5ccnc45)c3F)ccc12] | 6-(2,6-difluoro-3-(imidazo- [1,2-a]pyridine-8-sulfon- amido)phenyl)-7-fluoro-N- (3-hydroxypropyl)-1H- indazole-3-carboxamide | 545 (M + H)⁺ |
| 3AS | 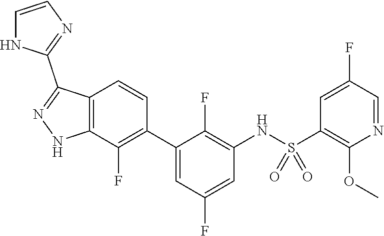 | N-(2,5-difluoro-3-(7- fluoro-3-(1H-imidazol-2- yl)-1H-indazol-6- yl)phenyl)-5-fluoro-2- methoxypyridine-3- sulfonamide | 549 (M + H)⁺ |
| 3AT | 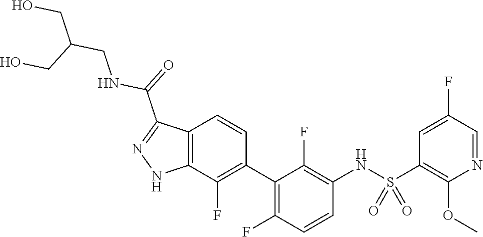 [SMILES: COc1ncc(F)cc1S(=O)(=O)Nc1ccc(F)c(-c2ccc3c(C(=O)NCC(CO)CO)n[nH]c3c2F)c1F] | 6-(2,6-difluoro-3-((5- fluoro-2-methoxypyridine)- 3-sulfonamido)phenyl)-7- fluoro-N-(3-hydroxy-2- (hydroxymethyl)propyl)- 1H-indazole-3- carboxamide | 584 (M + H)⁺ |
| 3AU | 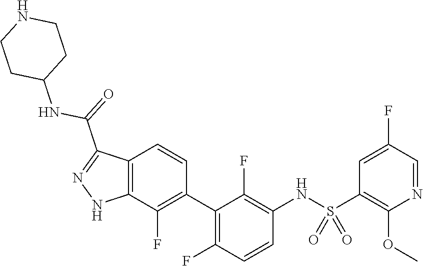 [SMILES: COc1ncc(F)cc1S(=O)(=O)Nc1ccc(F)c(-c2ccc3c(C(=O)NC4CCNCC4)n[nH]c3c2F)c1F] | 6-(2,6-difluoro-3-((5- fluoro-2-methoxypyridine)- 3-sulfonamido)phenyl)-7- fluoro-N-(piperidin-4-yl)- 1H-indazole-3- carboxamide | 579 (M + H)⁺ |
| 3AV | 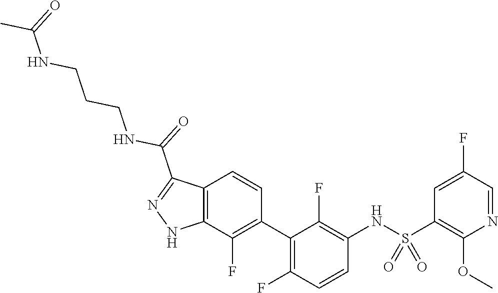 [SMILES: COc1ncc(F)cc1S(=O)(=O)Nc1ccc(F)c(-c2ccc3c(C(=O)NCCCNC(C)=O)n[nH]c3c2F)c1F] | N-(3-acetamidopropyl)-6- (2,6-difluoro-3-((5-fluoro- 2-methoxypyridine)-3- sulfon-amido)phenyl)-7- fluoro-1H-indazole-3- carboxamide | 595 (M + H)⁺ |
| 3AW | 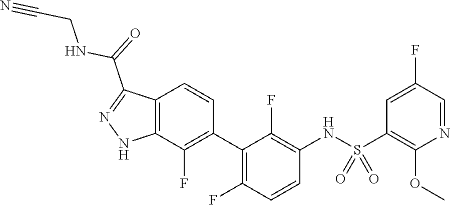 [SMILES: COc1ncc(F)cc1S(=O)(=O)Nc1ccc(F)c(-c2ccc3c(C(=O)NCC#N)n[nH]c3c2F)c1F] | N-(cyanomethyl)-6-(2,6- difluoro-3-((5-fluoro-2- methoxypyridine)-3-sulfon- amido)phenyl)-7-fluoro- 1H-indazole-3- carboxamide | 535 (M + H)⁺ |
| 3AX | 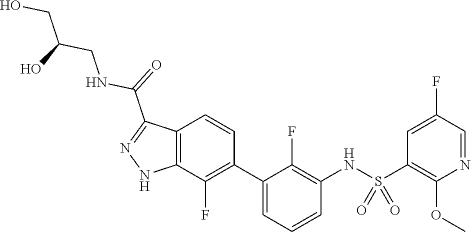 [SMILES: COc1ncc(F)cc1S(=O)(=O)Nc1cccc(-c2ccc3c(C(=O)NC[C@@H](O)CO)n[nH]c3c2F)c1F] | 6-(2-fluoro-3-((5-fluoro-2- methoxypyridine)-3- sulfonamido)phenyl)-N- ((R)-2,3-dihydroxypropyl)- 7-fluoro-1H-indazole-3- carboxamide | 552 (M + H)⁺ |
| 3AY | 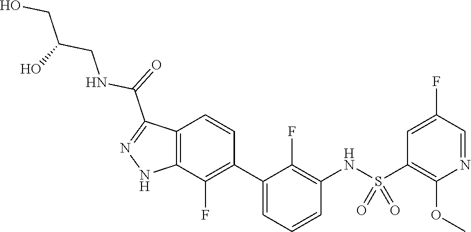 [SMILES: COc1ncc(F)cc1S(=O)(=O)Nc1cccc(-c2ccc3c(C(=O)NC[C@H](O)CO)n[nH]c3c2F)c1F] | 6-(2-fluoro-3-((5-fluoro-2- methoxypyridine)-3- sulfonamido)phenyl)-N- ((S)-2,3-dihydroxypropyl)- 7-fluoro-1H-indazole-3- carboxamide | 552 (M + H)⁺ |
| 3AZ | 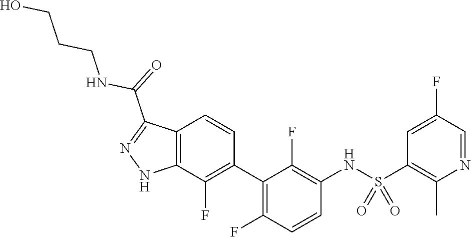 [SMILES: Cc1ncc(F)cc1S(=O)(=O)Nc1ccc(F)c(-c2ccc3c(C(=O)NCCCO)n[nH]c3c2F)c1F] | 6-(2,6-difluoro-3-((5- fluoro-2-methylpyridine)- 3-sulfonamido)phenyl)-7- fluoro-N-(3- hydroxypropyl)-1H- indazole-3-carboxamide | 538 (M + H)⁺ |
| 3BA | 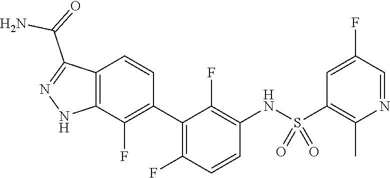 [SMILES: Cc1ncc(F)cc1S(=O)(=O)Nc1ccc(F)c(-c2ccc3c(C(N)=O)n[nH]c3c2F)c1F] | 6-(2,6-difluoro-3-((5- fluoro-2-methylpyridine)- 3-sulfon-amido)phenyl)-7- fluoro-1H-indazole-3- carboxamide | 480 (M + H)⁺ |
| 3BB | 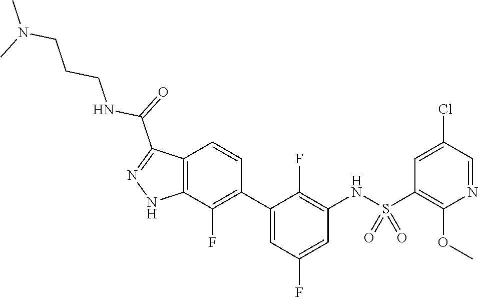 | 6-(3-((5-chloro-2-methoxy- pyridine)-3-sulfonamido)- 2,5-difluorophenyl)-N-(3- (dimethylamino)propyl)-7- fluoro-1H-indazole-3- carboxamide | 597 (M + H)⁺ |
| 3BC | 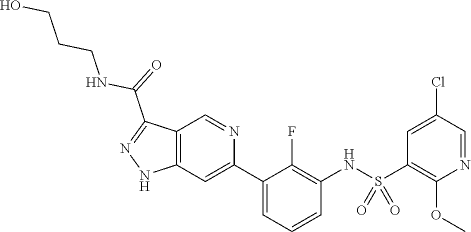 [SMILES: COc1ncc(Cl)cc1S(=O)(=O)Nc1cccc(-c2cc3[nH]nc(C(=O)NCCCO)c3cn2)c1F] | 6-(3-((5-chloro-2-methoxy- pyridine)-3-sulfonamido)- 2-fluorophenyl)-N-(3- hydroxy-propyl)-1H- pyrazolo[4,3-c]pyridine-3- carboxamide | 535 (M + H)⁺ |
| 3BD | 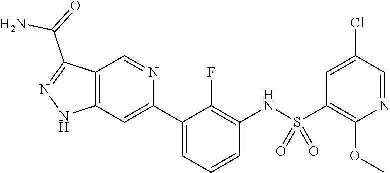 [SMILES: COc1ncc(Cl)cc1S(=O)(=O)Nc1cccc(-c2cc3[nH]nc(C(N)=O)c3cn2)c1F] | 6-(3-((5-chloro-2-methoxy- pyridine)-3-sulfonamido)- 2-fluorophenyl)-1H- pyrazolo-[4,3-c]pyridine-3- carboxamide | 477 (M + H)⁺ |
| 3BE | 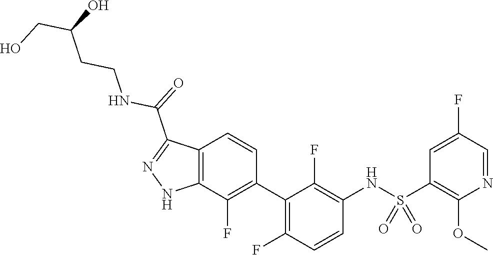 [SMILES: COc1ncc(F)cc1S(=O)(=O)Nc1ccc(F)c(-c2ccc3c(C(=O)NCC[C@H](O)CO)n[nH]c3c2F)c1F] | 6-(2,6-difluoro-3-((5- fluoro-2-methoxypyridine)- 3-sulfonamido)phenyl)-N- ((S)-3,4-dihydroxybutyl)-7- fluoro-1H-indazole-3- carboxamide | 584 (M + H)⁺ |
| 3BF | 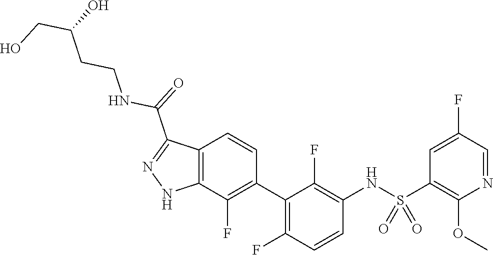 [SMILES: COc1ncc(F)cc1S(=O)(=O)Nc1ccc(F)c(-c2ccc3c(C(=O)NCC[C@@H](O)CO)n[nH]c3c2F)c1F] | 6-(2,6-difluoro-3-((5- fluoro-2-methoxypyridine)- 3-sulfonamido)phenyl)-N- ((R)-3,4-dihydroxybutyl)-7- fluoro-1H-indazole-3- carboxamide | 584 (M + H)⁺ |
| 3BG | 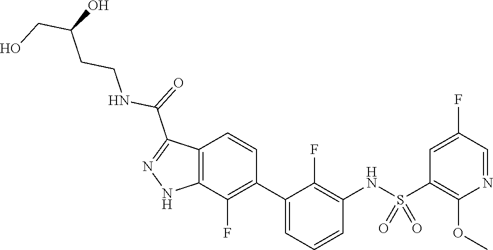 [SMILES: COc1ncc(F)cc1S(=O)(=O)Nc1cccc(-c2ccc3c(C(=O)NCC[C@H](O)CO)n[nH]c3c2F)c1F] | 6-(2-fluoro-3-((5-fluoro-2- methoxypyridine)-3- sulfonamido)phenyl)-N- ((S)-3,4-dihydroxybutyl)-7- fluoro-1H-indazole-3- carboxamide | 566 (M + H)⁺ |
| 3BH | 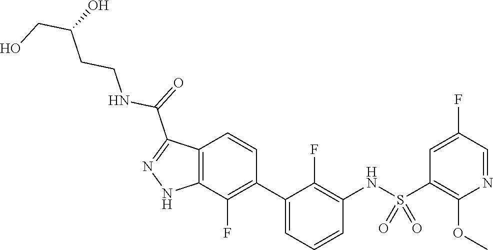 [SMILES: COc1ncc(F)cc1S(=O)(=O)Nc1cccc(-c2ccc3c(C(=O)NCC[C@@H](O)CO)n[nH]c3c2F)c1F] | 6-(2-fluoro-3-((5-fluoro-2- methoxypyridine)-3- sulfonamido)phenyl)-N- ((R)-3,4-dihydroxybutyl)-7- fluoro-1H-indazole-3- carboxamide | 566 (M + H)⁺ |
| 3BI | 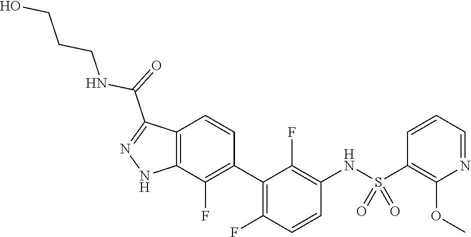 [SMILES: COc1ncccc1S(=O)(=O)Nc1ccc(F)c(-c2ccc3c(C(=O)NCCCO)n[nH]c3c2F)c1F] | 6-(2,6-difluoro-3-((2- methoxypyridine)-3- sulfonamido)phenyl)-7- fluoro-N-(3- hydroxypropyl)-1H- indazole-3-carboxamide | 536 (M + H)⁺ |
| 3BJ | 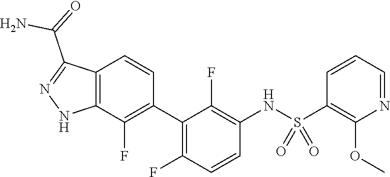 [SMILES: COc1ncccc1S(=O)(=O)Nc1ccc(F)c(-c2ccc3c(C(N)=O)n[nH]c3c2F)c1F] | 6-(2,6-difluoro-3-((2- methoxypyridine)-3- sulfonamido)phenyl)-7- fluoro-1H-indazole-3- carboxamide | 478 (M + H)⁺ |
| 3BK | 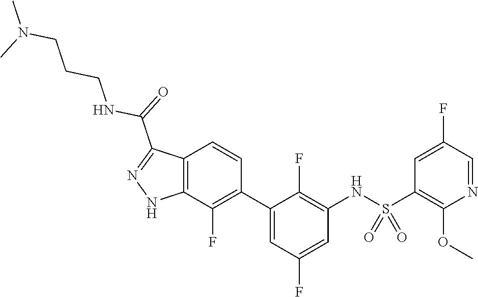 | 6-(3-((5-fluoro-2-methoxy- pyridine)-3-sulfonamido)- 2,5-difluorophenyl)-N-(3- (dimethylamino)propyl)-7- fluoro-1H-indazole-3- carboxamide | 581 (M + H)⁺ |
| 3BL | 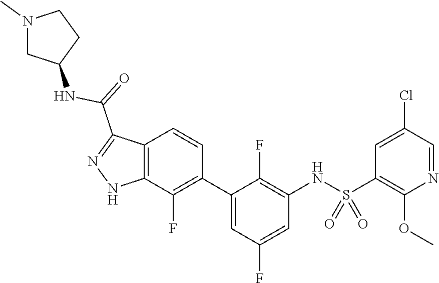 | 6-(3-((5-chloro-2-methoxy- pyridine)-3-sulfonamido)- 2,5-difluorophenyl)-7- fluoro-N-((R)-1- methylpyrrolidin-3-yl)-1H- indazole-3-carboxamide | 595 (M + H)⁺ |
| 3BM | 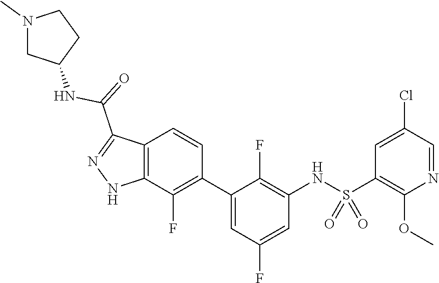 | 6-(3-((5-chloro-2-methoxy- pyridine)-3-sulfonamido)- 2,5-difluorophenyl)-7- fluoro-N-((S)-1- methylpyrrolidin-3-yl)-1H- indazole-3-carboxamide | 595 (M + H)⁺ |
| 3BN | 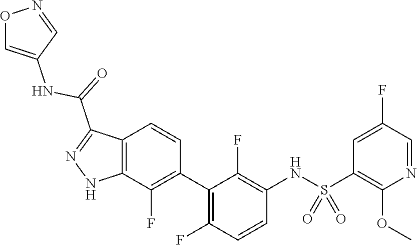 [SMILES: COc1ncc(F)cc1S(=O)(=O)Nc1ccc(F)c(-c2ccc3c(C(=O)Nc4cnoc4)n[nH]c3c2F)c1F] | 6-(2,6-difluoro-3-((5- fluoro-2-methoxypyridine)- 3-sulfonamido)phenyl)-7- fluoro-N-(isoxazol-4-yl)- 1H-indazole-3- carboxamide | 563 (M + H)⁺ |
| 3BO | 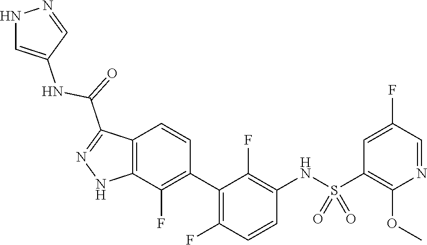 [SMILES: COc1ncc(F)cc1S(=O)(=O)Nc1ccc(F)c(-c2ccc3c(C(=O)Nc4cn[nH]c4)n[nH]c3c2F)c1F] | 6-(2,6-difluoro-3-((5- fluoro-2-methoxypyridine)- 3-sulfonamido)phenyl)-7- fluoro-N-(1H-pyrazol-4- yl)-1H-indazole-3- carboxamide | 562 (M + H)⁺ |
| 3BP | 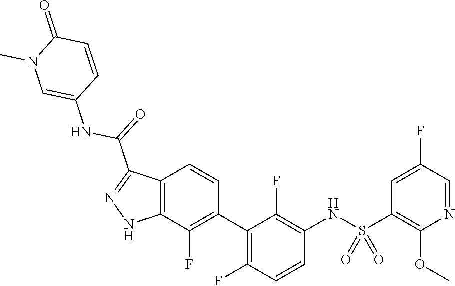 | 6-(2,6-difluoro-3-((5- fluoro-2-methoxypyridine)- 3-sulfonamido)phenyl)-7- fluoro-N-(1-methyl-6-oxo- 1,6-dihydropyridin-3-yl)- 1H-indazole-3- carboxamide | 603 (M + H)⁺ |
| 3BQ | 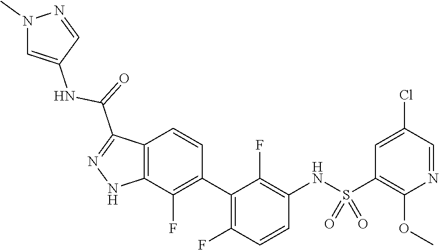 | 6-(3-((5-chloro-2-methoxy- pyridine)-3-sulfonamido)- 2,6-difluorophenyl)-7- fluoro-N-(1-methyl-1H- pyrazol-4-yl)-1H-indazole- 3-carboxamide | 592 (M + H)⁺ |
| 3BR | 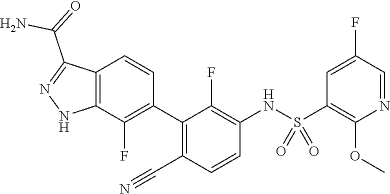 [SMILES: COc1ncc(F)cc1S(=O)(=O)Nc1ccc(C#N)c(-c2ccc3c(C(N)=O)n[nH]c3c2F)c1F] | 6-(6-cyano-2-fluoro-3-((5- fluoro-2-methoxypyridine)- 3-sulfonamido)phenyl)-7- fluoro-1H-indazole-3- carboxamide | 503 (M + H)⁺ |
| 3BS | 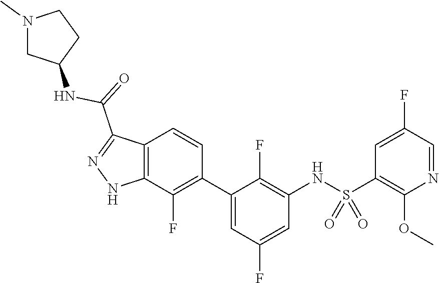 | 6-(3-((5-fluoro-2-methoxy- pyridine)-3-sulfonamido)- 2,5-difluorophenyl)-7- fluoro-N-((R)-1- methylpyrrolidin-3-yl)-1H- indazole-3-carboxamide | 579 (M + H)⁺ |
| 3BT | 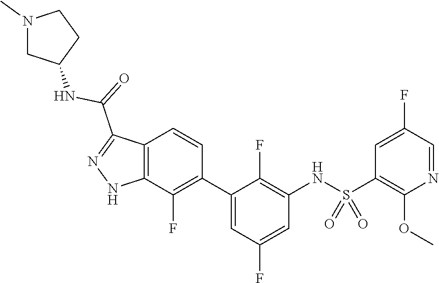 | 6-(3-((5-fluoro-2-methoxy- pyridine)-3-sulfonamido)- 2,5-difluorophenyl)-7- fluoro-N-((S)-1- methylpyrrolidin-3-yl)-1H- indazole-3-carboxamide | 579 (M + H)⁺ |
| 3BU | 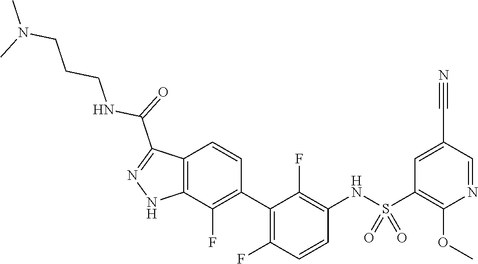 | 6-(3-((5-cyano-2-methoxy- pyridine)-3-sulfonamido)- 2,6-difluorophenyl)-N-(3- (dimethylamino)propyl)-7- fluoro-1H-indazole-3- carboxamide | 588 (M + H)⁺ |
| 3BV | 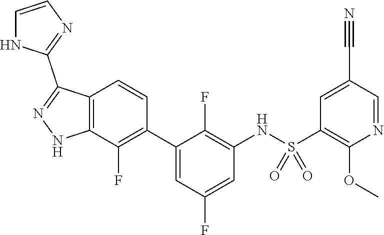 [SMILES: COc1ncc(C#N)cc1S(=O)(=O)Nc1cc(F)cc(-c2ccc3c(-c4ncc[nH]4)n[nH]c3c2F)c1F] | 5-cyano-N-(2,5-difluoro-3- (7-fluoro-3-(1H-imidazol- 2-yl)-1H-indazol-6- yl)phenyl)-2- methoxypyridine-3- sulfonamide | 526 (M + H)⁺ |
| 3BW | 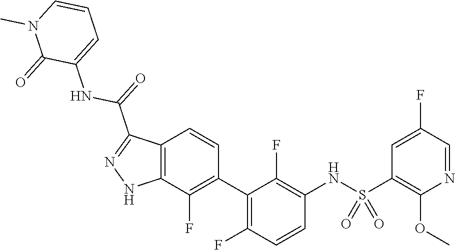 | 6-(2,6-difluoro-3-((5- fluoro-2-methoxypyridine)- 3-sulfonamido)phenyl)-7- fluoro-N-(1-methyl-2-oxo- 1,2-dihydropyridin-3-yl)- 1H-indazole-3- carboxamide | 603 (M + H)⁺ |
| 3BX | 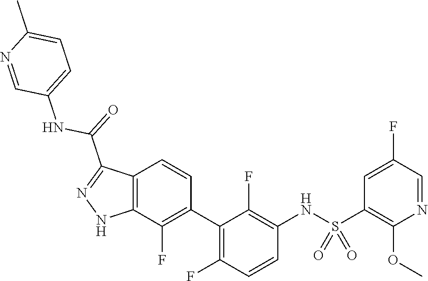 [SMILES: COc1ncc(F)cc1S(=O)(=O)Nc1ccc(F)c(-c2ccc3c(C(=O)Nc4ccc(C)nc4)n[nH]c3c2F)c1F] | 6-(2,6-difluoro-3-((5- fluoro-2-methoxypyridine)- 3-sulfonamido)phenyl)-7- fluoro-N-(6-methylpyridin- 3-yl)-1H-indazole-3- carboxamide | 587 (M + H)⁺ |
| 3BY | 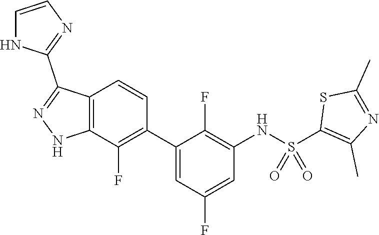 [SMILES: Cc1nc(C)c(S(=O)(=O)Nc2cc(F)cc(-c3ccc4c(-c5ncc[nH]5)n[nH]c4c3F)c2F)s1] | N-(2,5-difluoro-3-(7- fluoro-3-(1H-imidazol-2- yl)-1H-indazol-6- yl)phenyl)-2,4- dimethylthiazole-5- sulfonamide | 505 (M + H)⁺ |
| 3BZ | 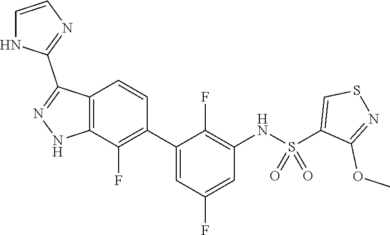 [SMILES: COc1nscc1S(=O)(=O)Nc1cc(F)cc(-c2ccc3c(-c4ncc[nH]4)n[nH]c3c2F)c1F] | N-(2,5-difluoro-3-(7- fluoro-3-(1H-imidazol-2- yl)-1H-indazol-6- yl)phenyl)-3- methoxyisothiazole-4- sulfonamide | 507 (M + H)⁺ |
| 3CA | 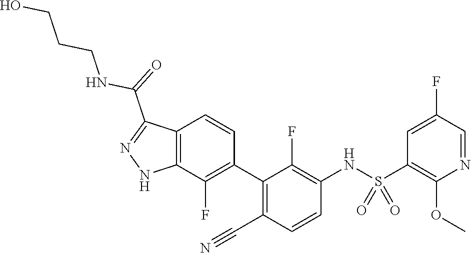 [SMILES: COc1ncc(F)cc1S(=O)(=O)Nc1ccc(C#N)c(-c2ccc3c(C(=O)NCCCO)n[nH]c3c2F)c1F] | 6-(6-cyano-2-fluoro-3-((5- fluoro-2-methoxypyridine)- 3-sulfonamido)phenyl)-7- fluoro-N-(3- hydroxypropyl)-1H- indazole-3-carboxamide | 561 (M + H)⁺ |
| 3CB | 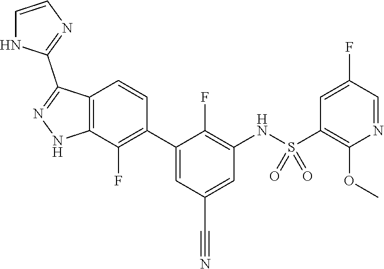 [SMILES: COc1ncc(F)cc1S(=O)(=O)Nc1cc(C#N)cc(-c2ccc3c(-c4ncc[nH]4)n[nH]c3c2F)c1F] | N-(5-cyano-2-fluoro-3-(7- fluoro-3-(1H-imidazol-2- yl)-1H-indazol-6- yl)phenyl)-5-fluoro-2- methoxypyridine-3- sulfonamide | 526 (M + H)⁺ |
| 3CC | 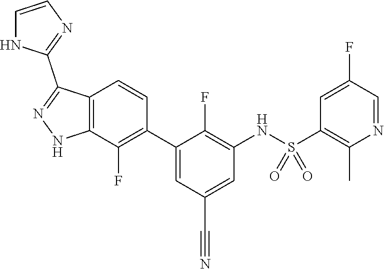 [SMILES: Cc1ncc(F)cc1S(=O)(=O)Nc1cc(C#N)cc(-c2ccc3c(-c4ncc[nH]4)n[nH]c3c2F)c1F] | N-(5-cyano-2-fluoro-3-(7- fluoro-3-(1H-imidazol-2- yl)-1H-indazol-6- yl)phenyl)-5-fluoro-2- methylpyridine-3- sulfonamide | 510 (M + H)⁺ |
| 3CD | 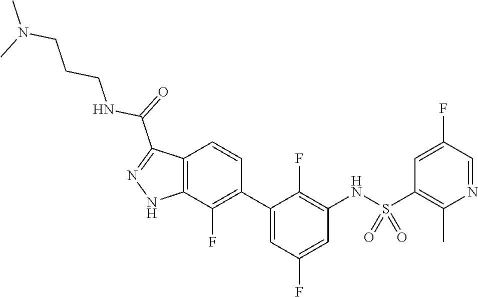 [SMILES: Cc1ncc(F)cc1S(=O)(=O)Nc1cc(F)cc(-c2ccc3c(C(=O)NCCCN(C)C)n[nH]c3c2F)c1F] | 6-(2,5-difluoro-3-((5- fluoro-2-methylpyridine)- 3-sulfonamido)phenyl)-N- (3-(dimethylamino)propyl)- 7-fluoro-1H-indazole-3- carboxamide | 565 (M + H)⁺ |
| 3CE | 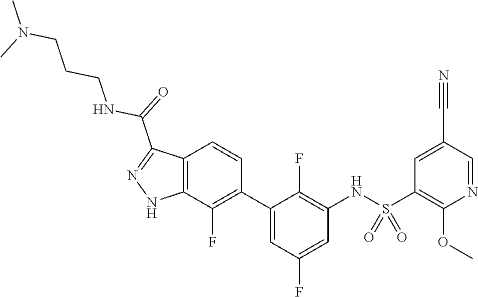 | 6-(3-((5-cyano-2-methoxy- pyridine)-3-sulfonamido)- 2,5-difluorophenyl)-N-(3- (dimethylamino)propyl)-7- fluoro-1H-indazole-3- carboxamide | 588 (M + H)⁺ |
| 3CF | 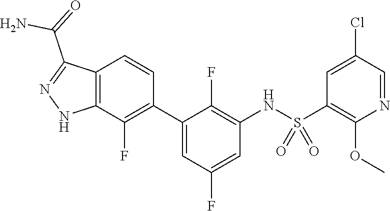 [SMILES: COc1ncc(Cl)cc1S(=O)(=O)Nc1cc(F)cc(-c2ccc3c(C(N)=O)n[nH]c3c2F)c1F] | 6-(3-((5-chloro-2-methoxy- pyridine)-3-sulfonamido)- 2,5-difluorophenyl)-7- fluoro-1H-indazole-3- carboxamide | 512 (M + H)⁺ |
| 3CG | 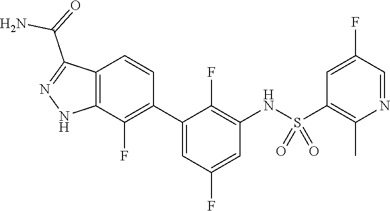 [SMILES: Cc1ncc(F)cc1S(=O)(=O)Nc1cc(F)cc(-c2ccc3c(C(N)=O)n[nH]c3c2F)c1F] | 6-(2,5-difluoro-3-((5- fluoro-2-methylpyridine)- 3-sulfon-amido)phenyl)-7- fluoro-1H-indazole-3- carboxamide | 480 (M + H)⁺ |
| 3CH | 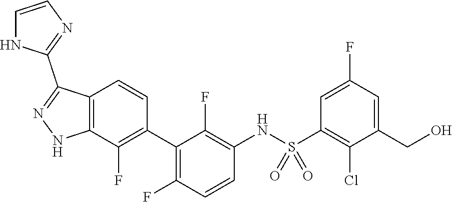 [SMILES: O=S(=O)(Nc1ccc(F)c(-c2ccc3c(-c4ncc[nH]4)n[nH]c3c2F)c1F)c1cc(F)cc(CO)c1Cl] | 2-chloro-N-(2,4-difluoro-3- (7-fluoro-3-(1H-imidazol- 2-yl)-1H-indazol-6- yl)phenyl)-5-fluoro-3- (hydroxymethyl)- benzenesulfonamide | 552 (M + H)⁺ |
| 3CI | 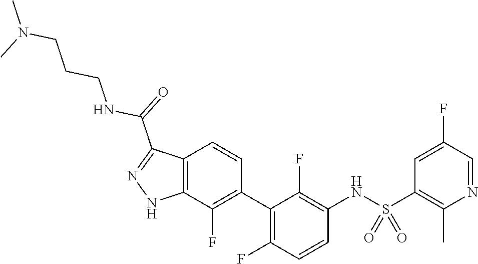 [SMILES: Cc1ncc(F)cc1S(=O)(=O)Nc1ccc(F)c(-c2ccc3c(C(=O)NCCCN(C)C)n[nH]c3c2F)c1F] | 6-(2,6-difluoro-3-((5- fluoro-2-methylpyridine)- 3-sulfonamido)phenyl)-N- (3-(dimethylamino)propyl)- 7-fluoro-1H-indazole-3- carboxamide | 565 (M + H)⁺ |
| 3CJ | 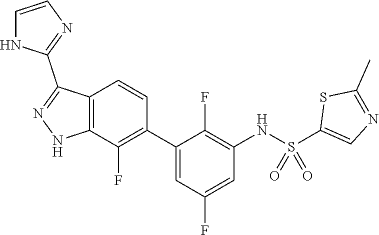 [SMILES: Cc1ncc(S(=O)(=O)Nc2cc(F)cc(-c3ccc4c(-c5ncc[nH]5)n[nH]c4c3F)c2F)s1] | N-(2,5-difluoro-3-(7- fluoro-3-(1H-imidazol-2- yl)-1H-indazol-6- yl)phenyl)-2- methylthiazole-5- sulfonamide | 491 (M + H)⁺ |
| 3CK | 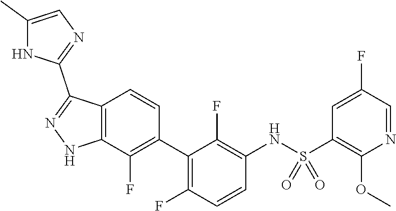 | N-(2,4-difluoro-3-(7- fluoro-3-(5-methyl-4H- 1,2,4-triazol-3-yl)-1H- indazol-6-yl)phenyl)-5- fluoro-2-methoxypyridine- 3-sulfonamide | 534 (M + H)⁺ |
| 3CL | 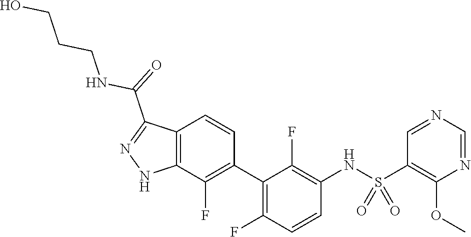 [SMILES: COc1ncncc1S(=O)(=O)Nc1ccc(F)c(-c2ccc3c(C(=O)NCCCO)n[nH]c3c2F)c1F] | 6-(2,6-difluoro-3-((4- methoxypyrimidine)-5- sulfonamido)phenyl)-7- fluoro-N-(3- hydroxypropyl)-1H- indazole-3-carboxamide | 537 (M + H)⁺ |
| 3CM | 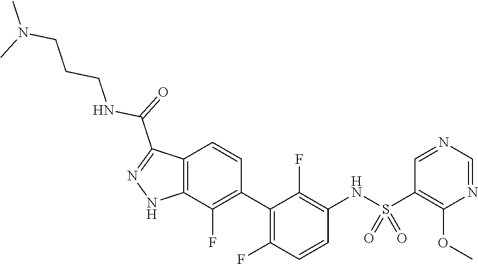 | 6-(2,6-difluoro-3-((4- methoxypyrimidine)-5- sulfonamido)phenyl)-N-(3- (dimethylamino)propyl)-7- fluoro-1H-indazole-3- carboxamide | 564 (M + H)⁺ |
| 3CN | 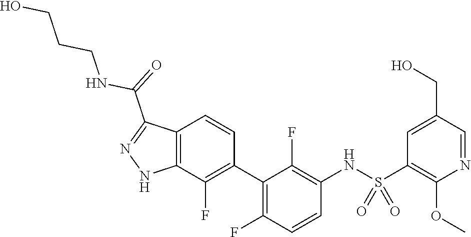 [SMILES: COc1ncc(CO)cc1S(=O)(=O)Nc1ccc(F)c(-c2ccc3c(C(=O)NCCCO)n[nH]c3c2F)c1F] | 6-(2,6-difluoro-3-((5- (hydroxylmethyl)-2- methoxy-pyridine)-3- sulfonamido)-phenyl)-7- fluoro-N-(3- hydroxypropyl)-1H- indazole-3-carboxamide | 566 (M + H)⁺ |
| 3CO | 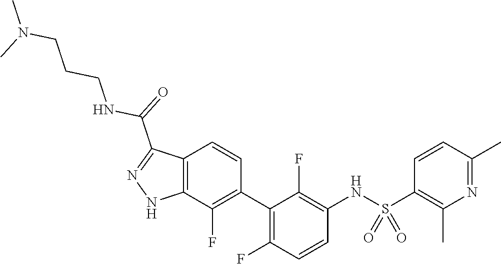 | N-(3- (dimethylamino)propyl)-6- (3-((2,6-dimethylpyridine)- 3-sulfonamido)-2,6- difluorophenyl)-7-fluoro- 1H-indazole-3- carboxamide | 561 (M + H)⁺ |
| 3CP | 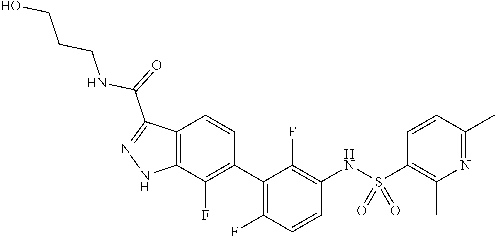 [SMILES: Cc1ccc(S(=O)(=O)Nc2ccc(F)c(-c3ccc4c(C(=O)NCCCO)n[nH]c4c3F)c2F)c(C)n1] | 6-(3-((2,6- dimethylpyridine)-3- sulfonamido)-2,6- difluorophenyl)-7-fluoro- N-(3-hydroxypropyl)-1H- indazole-3-carboxamide | 534 (M + H)⁺ |
| 3CQ | 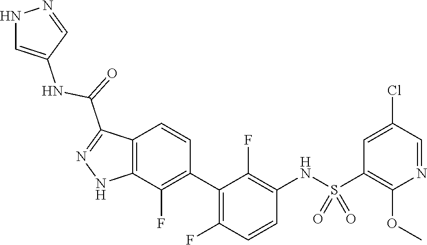 [SMILES: COc1ncc(Cl)cc1S(=O)(=O)Nc1ccc(F)c(-c2ccc3c(C(=O)Nc4cn[nH]c4)n[nH]c3c2F)c1F] | 6-(3-((5-chloro-2-methoxy- pyridine)-3-sulfonamido)- 2,6-difluorophenyl)-7- fluoro-N-(1H-pyrazol-4- yl)-1H-indazole-3- carboxamide | 578 (M + H)⁺ |
| 3CR | 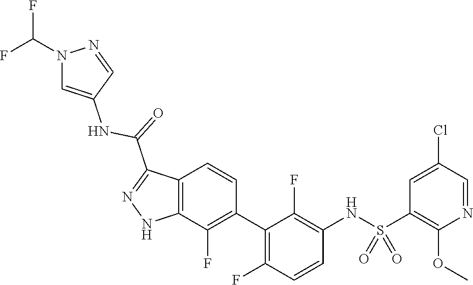 [SMILES: COc1ncc(Cl)cc1S(=O)(=O)Nc1ccc(F)c(-c2ccc3c(C(=O)Nc4cnn(C(F)F)c4)n[nH]c3c2F)c1F] | 6-(3-((5-chloro-2-methoxy- pyridine)-3-sulfonamido)- 2,6-difluorophenyl)-N-(1- (difluoromethyl)-1H pyrazol-4-yl)-7-fluoro-1H- indazole-3-carboxamide | 628 (M + H)⁺ |
| 3CS | 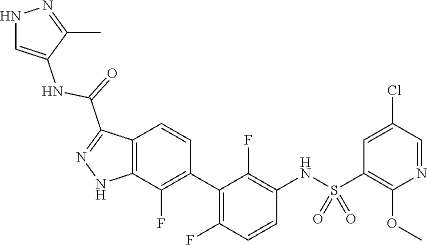 [SMILES: COc1ncc(Cl)cc1S(=O)(=O)Nc1ccc(F)c(-c2ccc3c(C(=O)Nc4c[nH]nc4C)n[nH]c3c2F)c1F] | 6-(3-((5-chloro-2-methoxy- pyridine)-3-sulfonamido)- 2,6-difluorophenyl)-7- fluoro-N-(3-methyl-1H- pyrazol-4-yl)-1H-indazole- 3-carboxamide | 592 (M + H)⁺ |
| 3CT | 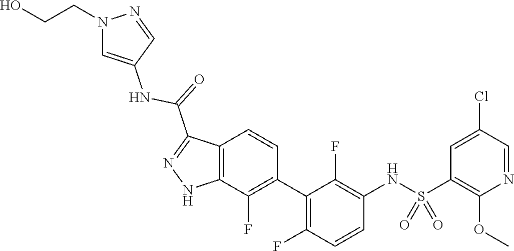 [SMILES: COc1ncc(Cl)cc1S(=O)(=O)Nc1ccc(F)c(-c2ccc3c(C(=O)Nc4cnn(CCO)c4)n[nH]c3c2F)c1F] | 6-(3-((5-chloro-2-methoxy- pyridine)-3-sulfonamido)- 2,6-difluorophenyl)-7- fluoro-N-(1-(2- hydroxyethyl)-1H-pyrazol- 4-yl)-1H-indazole-3- carboxamide | 622 (M + H)⁺ |
| 3CU | 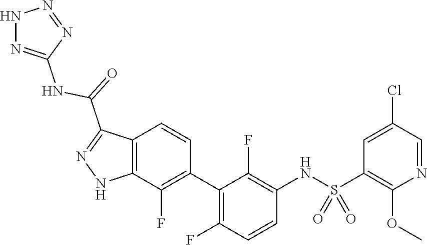 [SMILES: COc1ncc(Cl)cc1S(=O)(=O)Nc1ccc(F)c(-c2ccc3c(C(=O)Nc4nn[nH]n4)n[nH]c3c2F)c1F] | 6-(3-((5-chloro-2-methoxy- pyridine)-3-sulfonamido)- 2,6-difluorophenyl)-7- fluoro-N-(2H-tetrazol-5- yl)-1H-indazole-3- carboxamide | 580 (M + H)⁺ |
| 3CV | 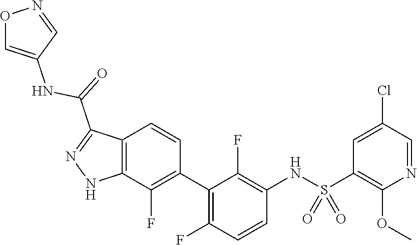 [SMILES: COc1ncc(Cl)cc1S(=O)(=O)Nc1ccc(F)c(-c2ccc3c(C(=O)Nc4cnoc4)n[nH]c3c2F)c1F] | 6-(3-((5-chloro-2-methoxy- pyridine)-3-sulfonamido)- 2,6-difluorophenyl)-7- fluoro-N-(isoxazol-4-yl)- 1H-indazole-3- carboxamide | 579 (M + H)⁺ |
| 3CW | 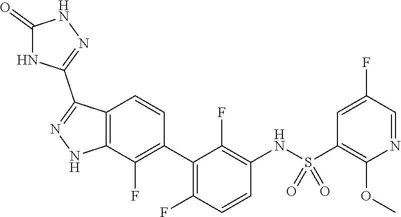 [SMILES: COc1ncc(F)cc1S(=O)(=O)Nc1ccc(F)c(-c2ccc3c(-c4n[nH]c(=O)[nH]4)n[nH]c3c2F)c1F] | N-(2,4-difluoro-3-(7- fluoro-3-(5-oxo-4,5- dihydro-1H-1,2,4-triazol-3- yl)-1H-indazol-6- yl)phenyl)-5-fluoro-2- methoxypyridine-3- sulfonamide | 536 (M + H)⁺ |
| 3CX | 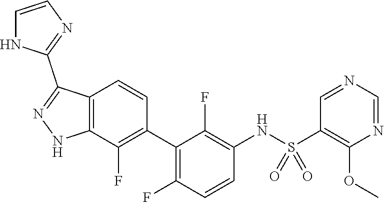 [SMILES: COc1ncncc1S(=O)(=O)Nc1ccc(F)c(-c2ccc3c(-c4ncc[nH]4)n[nH]c3c2F)c1F] | N-(2,4-difluoro-3-(7- fluoro-3-(1H-imidazol-2- yl)-1H-indazol-6- yl)phenyl)-4- methoxypyrimidine-5- sulfonamide | 502 (M + H)⁺ |
| 3CY | 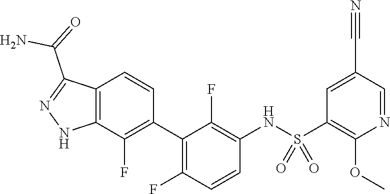 [SMILES: COc1ncc(C#N)cc1S(=O)(=O)Nc1ccc(F)c(-c2ccc3c(C(N)=O)n[nH]c3c2F)c1F] | 6-(3-((5-cyano-2-methoxy- pyridine)-3-sulfonamido)- 2,6-difluorophenyl)-7- fluoro-1H-indazole-3- carboxamide | 503 (M + H)⁺ |
| 3CZ | 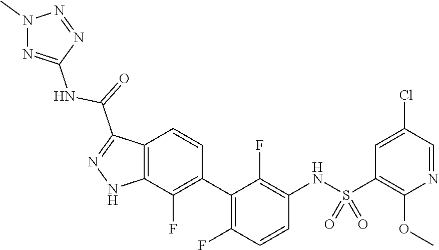 | 6-(3-((5-chloro-2-methoxy- pyridine)-3-sulfonamido)- 2,6-difluorophenyl)-7- fluoro-N-(2-methyl-2H- tetrazol-5-yl)-1H-indazole- 3-carboxamide | 594 (M + H)⁺ |
| 3DA | 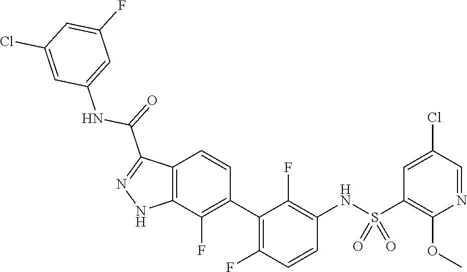 [SMILES: COc1ncc(Cl)cc1S(=O)(=O)Nc1ccc(F)c(-c2ccc3c(C(=O)Nc4cc(F)cc(Cl)c4)n[nH]c3c2F)c1F] | 6-(3-((5-chloro-2-methoxy- pyridine)-3-sulfonamido)- 2,6-difluorophenyl)-N-(3- chloro-5-fluorophenyl)-7- fluoro-1H-indazole-3- carboxamide | 638 (M + H)⁺ |
| 3DB | 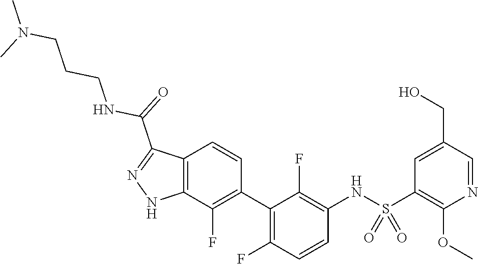 | 6-(2,6-difluoro-3-((5- (hydroxymethyl)-2-meth- oxypyridine)-3- sulfonamido)-phenyl)-N- (3-(dimethyl- amino)propyl)-7-fluoro- 1H-indazole-3- carboxamide | 593 (M + H)⁺ |
| 3DC | 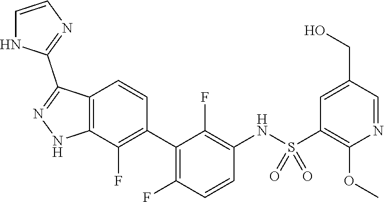 [SMILES: COc1ncc(CO)cc1S(=O)(=O)Nc1ccc(F)c(-c2ccc3c(-c4ncc[nH]4)n[nH]c3c2F)c1F] | N-(2,4-difluoro-3-(7- fluoro-3-(1H-imidazol-2- yl)-1H-indazol-6- yl)phenyl)-5- (hydroxylmethyl)-2- methoxypyridine-3- sulfonamide | 531 (M + H)⁺ |
| 3DD | 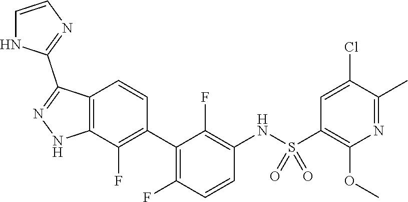 [SMILES: COc1nc(C)c(Cl)cc1S(=O)(=O)Nc1ccc(F)c(-c2ccc3c(-c4ncc[nH]4)n[nH]c3c2F)c1F] | 5-chloro-N-(2,4-difluoro-3- (7-fluoro-3-(1H-imidazol- 2-yl)-1H-indazol-6- yl)phenyl)-2-methoxy-6- methylpyridine-3- sulfonamide | 549 (M + H)⁺ |
| 3DE | 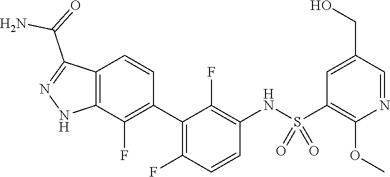 [SMILES: COc1ncc(CO)cc1S(=O)(=O)Nc1ccc(F)c(-c2ccc3c(C(N)=O)n[nH]c3c2F)c1F] | 6-(2,6-difluoro-3-((5- (hydroxylmethyl)-2- methoxy-pyridine)-3- sulfonamido)-phenyl)-7- fluoro-1H-indazole-3- carboxamide | 508 (M + H)⁺ |
| 3DF | 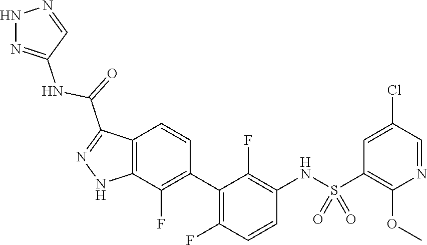 [SMILES: COc1ncc(Cl)cc1S(=O)(=O)Nc1ccc(F)c(-c2ccc3c(C(=O)Nc4cn[nH]n4)n[nH]c3c2F)c1F] | 6-(3-((5-chloro-2-methoxy- pyridine)-3-sulfonamido)- 2,6-difluorophenyl)-7- fluoro-1H-(2H-1,2,3-triazol- 4-yl)-1H-indazole-3- carboxamide | 579 (M + H)⁺ |
| 3DG | 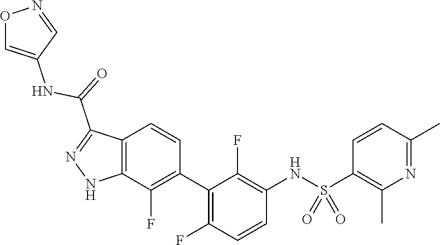 [SMILES: Cc1ccc(S(=O)(=O)Nc2ccc(F)c(-c3ccc4c(C(=O)Nc5cnoc5)n[nH]c4c3F)c2F)c(C)n1] | 6-(3-((2,6- dimethylpyridine)-3- sulfonamido)-2,6-difluoro- phenyl)-7-fluoro-N- (isoxazol-4-yl)-1H- indazole-3-carboxamide | 543 (M + H)⁺ |
| 3DH | 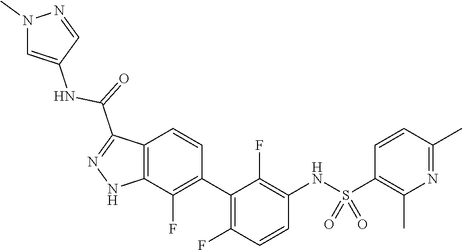 | 6-(3-((2,6- dimethylpyridine)-3- sulfonamido)-2,6-difluoro- phenyl)-7-fluoro-N-(1- methyl-1H-pyrazol-4-yl)- 1H-indazole-3- carboxamide | 556 (M + H)⁺ |
| 3DI | 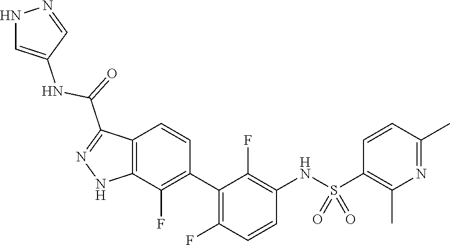 [SMILES: Cc1ccc(S(=O)(=O)Nc2ccc(F)c(-c3ccc4c(C(=O)Nc5cn[nH]c5)n[nH]c4c3F)c2F)c(C)n1] | 6-(3-((2,6- dimethylpyridine)-3- sulfonamido)-2,6-difluoro- phenyl)-7-fluoro-N-(1H- pyrazol-4-yl)-1H-indazole- 3-carboxamide | 542 (M + H)⁺ |
| 3DJ | 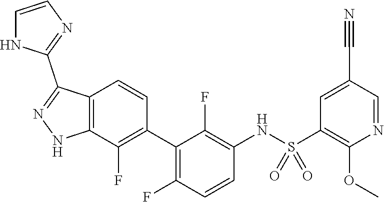 [SMILES: COc1ncc(C#N)cc1S(=O)(=O)Nc1ccc(F)c(-c2ccc3c(-c4ncc[nH]4)n[nH]c3c2F)c1F] | 5-cyano-N-(2,4-difluoro-3- (7-fluoro-3-(1H-imidazol- 2-yl)-1H-indazol-6- yl)phenyl)-2- methoxypyridine-3- sulfonamide | 526 (M + H)⁺ |
| 3DK | 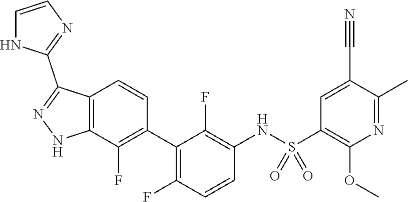 [SMILES: COc1nc(C)c(C#N)cc1S(=O)(=O)Nc1ccc(F)c(-c2ccc3c(-c4ncc[nH]4)n[nH]c3c2F)c1F] | 5-cyano-N-(2,4-difluoro-3- (7-fluoro-3-(1H-imidazol- 2-yl)-1H-indazol-6- yl)phenyl)-2-methoxy-6- methylpyridine-3- sulfonamide | 540 (M + H)⁺ |
| 3DL | 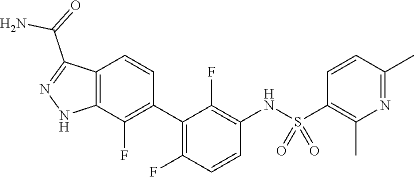 [SMILES: Cc1ccc(S(=O)(=O)Nc2ccc(F)c(-c3ccc4c(C(N)=O)n[nH]c4c3F)c2F)c(C)n1] | 6-(3-((2,6- dimethylpyridine)-3- sulfonamido)-2,6-difluoro- phenyl)-7-fluoro-1H- indazole-3-carboxamide | 476 (M + H)⁺ |
| 3DM | 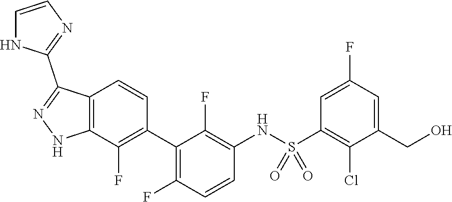 [SMILES: O=S(=O)(Nc1ccc(F)c(-c2ccc3c(-c4ncc[nH]4)n[nH]c3c2F)c1F)c1cc(F)cc(CO)c1Cl] | 2-chloro-N-(2,4-difluoro-3- (7-fluoro-3-(1H-imidazol- 2-yl)-1H-indazol-6- yl)phenyl)-5-fluoro-3- (hydroxymethyl)- benzenesulfonamide | 552 (M + H)⁺ |
| 3DN | 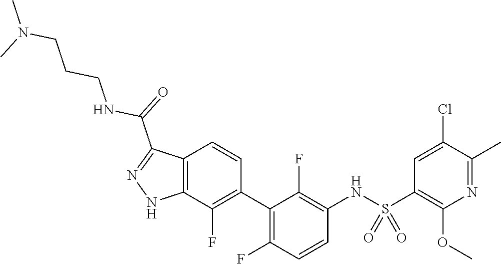 | 6-(3-((5-chloro-2-methoxy- 6-methylpyridine)-3- sulfon-amido)-2,6- difluorophenyl)-N-(3- (dimethylamino)propyl)-7- fluoro-1H-indazole-3- carboxamide | 611 (M + H)⁺ |
| 3DO | 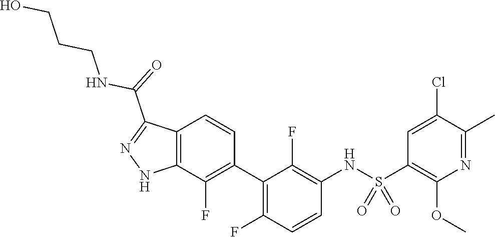 [SMILES: COc1nc(C)c(Cl)cc1S(=O)(=O)Nc1ccc(F)c(-c2ccc3c(C(=O)NCCCO)n[nH]c3c2F)c1F] | 6-(3-((5-chloro-2-methoxy- 6-methylpyridine)-3- sulfon-amido)-2,6- difluorophenyl)-7-fluoro- N-(3-hydroxypropyl)-1H- indazole-3-carboxamide | 584 (M + H)+ |
| 3DP | 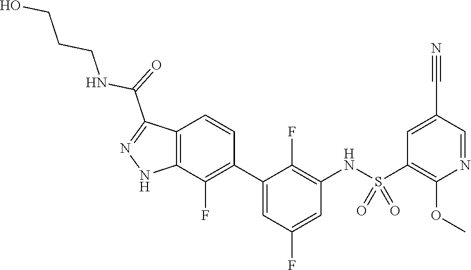 [SMILES: COc1ncc(C#N)cc1S(=O)(=O)Nc1cc(F)cc(-c2ccc3c(C(=O)NCCCO)n[nH]c3c2F)c1F] | 6-(3-((5-cyano-2-methoxy- pyridine)-3-sulfonamido)- 2,5-difluorophenyl)-7- fluoro-N-(3- hydroxypropyl)-1H- indazole-3-carboxamide | 561 (M + H)⁺ |
| 3DQ | 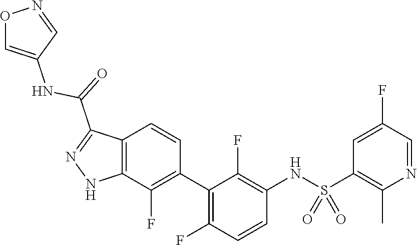 [SMILES: Cc1ncc(F)cc1S(=O)(=O)Nc1ccc(F)c(-c2ccc3c(C(=O)Nc4cnoc4)n[nH]c3c2F)c1F] | 6-(2,6-difluoro-3-((5- fluoro-2-methylpyridine)- 3-sulfonamido)phenyl)-7- fluoro-N-(isoxazol-4-yl)- 1H-indazole-3- carboxamide | 547 (M + H)⁺ |
| 3DR | 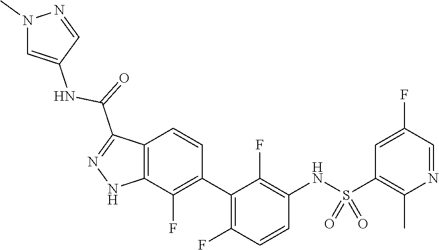 [SMILES: Cc1ncc(F)cc1S(=O)(=O)Nc1ccc(F)c(-c2ccc3c(C(=O)Nc4cnn(C)c4)n[nH]c3c2F)c1F] | 6-(2,6-difluoro-3-((5- fluoro-2-methylpyridine)- 3-sulfon-amido)phenyl)-7- fluoro-N-(1-methyl-1H- pyrazol-4-yl)-1H-indazole- 3-carboxamide | 560 (M + H)⁺ |
| 3DS | 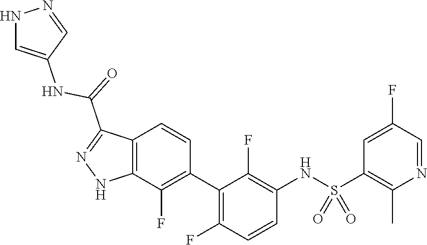 [SMILES: Cc1ncc(F)cc1S(=O)(=O)Nc1ccc(F)c(-c2ccc3c(C(=O)Nc4cn[nH]c4)n[nH]c3c2F)c1F] | 6-(2,6-difluoro-3-((5- fluoro-2-methylpyridine)- 3-sulfonamido)phenyl)-7- fluoro-N-(1H-pyrazol-4- yl)-1H-indazole-3- carboxamide | 546 (M + H)⁺ |

TABLE 6

| Compd | |
|---|---|
| No. | Physical Characterization Data |
| 3A | ¹H NMR (400 MHz, CD₃OD-d₄) δ 8.38 (d, J = 2.4 Hz, 1H), 8.02-8.06 (m, |
| | 2H), 7.75 (s, 2H), 7.52 (m, 1H), 7.35 (m, 1H), 7.20 (m, 1H), 4.06 (s, 3H). |
| 3B | ¹H NMR (400 MHz, CD₃OD-d₄) δ 8.32 (s, 1H), 8.10 (s, 1H), 7.90 (d, J = 7.4 |
| | Hz, 1H), 7.55 (m, 1H), 7.05-7.15 (m, 2H), 4.90 (m, 1H), 4.80 (m, 1H), 4.10 |
| | (m, 1H), 3.96 (s, 3H), 3.75 (m, 1H), 3.65 (m, 1H). |
| 3C | ¹H NMR (400 MHz, CD₃OD-d₄) δ 8.36 (s, 1H), 8.19 (s, 1H), 8.15 (d, J = 8.3 |
| | Hz, 1H), 7.38 (ddd, J = 9.3, 5.9, 3.2 Hz, 1H), 7.24 (s, 2H), 7.15 (dd, J = 8.4, |
| | 5.9 Hz, 1H), 7.08 (ddd, J = 8.3, 5.1, 3.2 Hz, 1H), 4.00 (s, 3H). |
| 3D | ¹H NMR (400 MHz, CD₃OD-d₄) δ 8.30 (s, 1H), 8.09 (s, 1H), 8.07 (d, J = |
| | 12.4 Hz, 1H), 7.54 (m, 1H), 7.05-7.15 (m, 2H), 3.98 (s, 3H), 3.75 (m, 1H), |
| | 3.45 (m, 1H), 3.17 (m, 1H), 2.69 (m, 1H), 2.50 (s, 3H), 2.40 (m, 1H), 2.06 (m, |
| | 1H), 1.75-1.90 (m, 3H). |
| 3E | ¹H NMR (400 MHz, CD₃OD-d₄) δ 8.30 (s, 1H), 8.09 (s, 1H), 8.07 (d, J = |
| | 12.4 Hz, 1H), 7.54 (m, 1H), 7.05-7.15 (m, 2H), 3.98 (s, 3H), 3.75 (m, 1H), |
| | 3.45 (m, 1H), 3.17 (m, 1H), 2.69 (m, 1H), 2.50 (s, 3H), 2.40 (m, 1H), 2.06 (m, |
| | 1H), 1.75-1.90 (m, 3H). |
| 3F | ¹H NMR (400 MHz, CD₃OD-d₄) δ 8.51 (s, 1H), 8.26 (d, 1H), 8.20 (d, 1H), |
| | 7.97 (s, 1H), 7.26 (s, 2H), 7.22 (m, 1H), 2.89 (s, 3H). |
| 3G | ¹H NMR (400 MHz, CD₃OD-d₄) δ 8.35-8.38 (m, 3H), 8.15 (d, 1H), 7.84 (d, |
| | 1H), 7.77 (s, 2H), 7.60 (m, 1H), 7.16 (m, 1H), 4.02 (s, 3H). |
| 3H | ¹H NMR (400 MHz, CD₃OD-d₄) δ 8.17 (dd, 1H), 7.83 (s, 1H), 7.72 (m, 1H), |
| | 7.25 (s, 2H), 7.14-7.25 (m, 3H). |
| 3I | ¹H NMR (400 MHz, CD₃OD-d₄) δ 8.75 (s, 1H), 8.42 (s, 1H), 8.08 (d, 1H), |
| | 7.62 (m, 1H), 7.08-7.18 (m, 2H), 4.10 (s, 3H). |
| 3J | ¹H NMR (400 MHz, CD₃OD-d₄) δ 8.27-8.41 (m, 3H), 8.14 (d, J = 8.4 Hz, |
| | 1H), 7.75 (s, 2H), 7.52 (dd, J = 8.6, 5.9 Hz, 1H), 7.20-7.20 (m, 2H), 3.97 (s, |
| | 3H). |
| 3K | ¹H NMR (400 MHz, CD₃OD-d₄) δ ppm 8.30 (d, J = 2.93 Hz, 1H), 8.02-8.18 |
| | (m, 3H), 7.62 (s, 1H), 7.41 (br dd, J = 8.51, 5.77 Hz, 1H), 4.71 (s, 2H), 4.02 |
| | (s, 3H). |
| 3L | ¹H NMR (400 MHz, CD₃OD-d₄) δ 7.90-7.97 (m, 2H), 7.88 (s, 1H), 7.82 (dd, |
| | J = 7.9, 1.4 Hz, 1H), 7.26 (s, 2H), 6.95-7.03 (m, 2H), 2.52 (s, 3H). |
| 3M | ¹H NMR (400 MHz, CD₃OD-d₄) δ 7.88 (d, J = 7.8 Hz, 1H), 7.80 (s, 1H), |
| | 7.55 (s, 1H), 7.42 (d, J = 6.8 Hz, 1H), 7.05 (m, 1H), 3.68 (s, 3H). |
| 3N | ¹H NMR (400 MHz, CD₃OD-d₄) δ 8.55 (s, 1H), 8.14 (d, J = 8.4 Hz, 1H), |
| | 7.97 (dd, J = 8.1, 2.8 Hz, 1H), 7.75 (s, 2H), 7.63 (td, J = 7.6, 1.8 Hz, 1H), |
| | 7.15-7.41 (m, 3H), 2.83 (s, 3H). |
| 3O | ¹H NMR (400 MHz, CD₃OD-d₄) δ 8.79 (d, J = 7.2 Hz, 1H), 7.97 (d, J = 8.6 |
| | Hz, 2H), 7.76 (m, 1H), 7.75 (s, 2H), 7.59-7.67 (m, 2H), 7.16-7.27 (m, 2H), |
| | 7.10 (m, 1H). |
| 3P | ¹H NMR (400 MHz, CD₃OD-d₄) δ 8.22 (s, 1H), 8.12 (dd, J = 8.4, 0.9 Hz, |
| | 1H), 7.68 (s, 1H), 7.39 (s, 1H), 7.34 (dd, J = 7.3, 3.0 Hz, 1H), 7.08 (dt, J = |
| | 8.4, 1.3 Hz, 1H), 3.88 (s, 3H). |
| 3Q | ¹H NMR (400 MHz, CD₃OD-d₄) δ 8.29 (s, 1H), 8.19 (dd, J = 7.3, 3.0 Hz, |
| | 1H), 8.15 (d, J = 8.4 Hz, 1H), 7.99 (s, 1H), 7.29 (dd, J = 8.4, 5.9 Hz, 1H), |
| | 7.17 (t, J = 5.3 Hz, 1H), 3.98 (s, 3H). |
| 3R | ¹H NMR (400 MHz, CD₃OD-d₄) δ 8.25 (s, 1H), 8.07 (d, J = 7.8 Hz, 1H), |
| | 7.95 (m, 1H), 7.55 (m, 1H), 7.14-7.05 (m, 2H), 3.97 (s, 3H), 3.70 (t, J = 6.2 |
| | Hz, 2H), 3.65 (t, J = 5.6 Hz, 2H), 1.90 (m, 2H). |
| 3S | ¹H NMR (400 MHz, CD₃OD-d₄) δ 8.26 (s, 1H), 8.07 (d, J = 7.8 Hz, 1H), |
| | 7.95 (m, 1H), 7.57 (m, 1H), 7.06-7.12 (m, 2H), 3.98 (s, 3H), 3.00 (s, 3H). |
| 3T | ¹H NMR (400 MHz, CD₃OD-d₄) δ 8.27 (s, 1H), 8.07 (d, J = 7.8 Hz, 1H), |
| | 7.95 (d, J = 5.6 Hz, 1H), 7.56 (m, 1H), 7.06-7.12 (m, 2H), 3.99 (s, 3H). |
| 3U | ¹H NMR (400 MHz, CD₃OD-d₄) δ 8.22 (s, 1H), 8.10 (d, J = 5.6 Hz, 1H), |
| | 7.95 (d, J = 5.6 Hz, 1H), 7.58 (m, 1H), 7.05-7.15 (m, 2H), 3.95 (s, 3H), 3.55 |
| | (t, J = 5.6 Hz, 2H), 2.55 (m, 2H), 2.35 (s, 6H), 1.90 (m, 2H). |
| 3V | ¹H NMR (400 MHz, CD₃OD-d₄) δ 8.27 (s, 1H), 7.94-8.05 (m, 2H), 7.74 (s, |
| | 2H), 7.60 (m, 1H), 7.24-7.37 (m, 3H), 4.01 (s, 3H). |
| 3W | ¹H NMR (400 MHz, CD₃OD-d₄) δ 8.35 (dd, J = 5.0, 1.9 Hz, 1H), 8.15 (dd, |
| | J = 7.6, 1.9 Hz, 1H), 8.00 (d, J = 8.4 Hz, 1H), 7.74 (s, 2H), 7.59 (td, J = 7.4, 2.4 |
| | Hz, 1H), 7.20-7.36 (m, 3H), 7.06 (dd, J = 7.6, 5.0 Hz, 1H), 4.03 (s, 3H). |
| 3Y | ¹H NMR (400 MHz, CD₃OD-d₄) δ 8.54 (d, J = 2.8 Hz, 1H), 8.12 (d, J = 8.3 |
| | Hz, 1H), 7.96 (dd, J = 8.1, 2.8 Hz, 1H), 7.54 (m, 1H), 7.10-7.36 (m, 4H), 6.99 |
| | (d, J = 8.4 Hz, 1H), 2.85 (s, 3H), 2.15 (s, 3H). |
| 3Z | ¹H NMR (400 MHz, CD₃OD-d₄) δ 8.26 (s, 1H), 8.12 (d, J = 5.6 Hz, 1H), |
| | 8.10 (s, 1H), 7.95 (d, J = 5.6 Hz, 1H), 7.75 (s, 1H), 7.58 (m, 1H), 7.02-7.18 |
| | (m, 2H), 3.98 (s, 3H), 3.92 (s, 3H). |
| 3AA | ¹H NMR (400 MHz, CD₃OD-d₄) δ 8.83 (d, J = 6.9 Hz, 1H), 8.13 (s, 1H), |
| | 8.01 (d, J = 8.4 Hz, 1H), 7.84 (d, J = 9.1 Hz, 1H), 7.75 (m, 1H), 7.64 (td, J = |
| | 8.8, 5.7 Hz, 1H), 7.35 (m, 1H), 7.18 (td, J = 8.9, 1.8 Hz, 1H), 6.92 (dd, J = |
| | 8.4, 5.7 Hz, 1H). |
| 3AB | ¹H NMR (400 MHz, CD₃OD-d₄) δ 8.26 (s, 1H), 8.10 (d, J = 5.6 Hz, 1H), |
| | 7.95 (d, J = 5.6 Hz, 1H), 7.58 (m, 1H), 7.02-7.18 (m, 2H), 4.70 (m, 1H), 4.00 |
| | (s, 3H), 3.00-2.88 (m, 2H), 2.76 (m, 1H), 2.67 (m, 1H), 2.50 (s, 3H), 2.44 (m, |
| | 1H), 1.94 (m, 1H). |
| 3AC | ¹H NMR (400 MHz, CD₃OD-d₄) δ 8.26 (s, 1H), 8.10 (d, J = 5.6 Hz, 1H), |
| | 7.95 (d, J = 5.6 Hz, 1H), 7.58 (m, 1H), 7.02-7.18 (m, 2H), 4.70 (m, 1H), 4.00 |
| | (s, 3H), 3.00-2.88 (m, 2H), 2.76 (m, 1H), 2.67 (m, 1H), 2.50 (s, 3H), 2.44 (m, |
| | 1H), 1.94 (m, 1H). |
| 3AD | ¹H NMR (400 MHz, CD₃OD-d₄) δ 8.33 (s, 1H), 8.04-8.09 (m, 2H), 7.57 (m, |
| | 1H), 7.05-7.15 (m, 2H), 4.72 (m, 1H), 4.00 (s, 3H), 3.03 (m, 2H), 2.80 (m, |
| | 1H), 2.65 (m, 1H), 2.50 (s, 3H), 2.46 (m, 1H), 1.95 (m, 1H). |
| 3AF | ¹H NMR (400 MHz, CD₃OD-d₄) δ 8.33 (s, 1H), 8.04-8.09 (m, 2H), 7.57 (m, |
| | 1H), 7.05-7.15 (m, 2H), 4.72 (m, 1H), 4.00 (s, 3H), 3.03 (m, 2H), 2.80 (m, |
| | 1H), 2.65 (m, 1H), 2.50 (s, 3H), 2.46 (m, 1H), 1.95 (m, 1H). |
| 3AH | ¹H NMR (400 MHz, CD₃OD-d₄) δ 8.25 (s, 1H), 8.07 (d, 1H), 8.01 (d, J = 7.8 |
| | Hz, 1H), 7.55 (m, 1H), 7.05-7.14 (m, 2H), 4.00 (s, 3H), 3.89 (m, 1H), 3.75- |
| | 3.55 (m, 3H), 3.45 (m, 1H). |
| 3AI | ¹H NMR (400 MHz, CD₃OD-d₄) δ 8.25 (s, 1H), 8.07 (d, 1H), 8.01 (d, J = 7.8 |
| | Hz, 1H), 7.55 (m, 1H), 7.05-7.14 (m, 2H), 4.00 (s, 3H), 3.89 (m, 1H), 3.75- |
| | 3.55 (m, 3H), 3.45 (m, 1H). |
| 3AJ | ¹H NMR (400 MHz, DMSO-d₆) δ 14.35 (bs, 1H), 10.05 (bs, 1H), 8.47 (d, J = |
| | 6.8 Hz, 1H), 8.35 (s, 1H), 8.02 (d, J = 5.6 Hz, 1H), 7.95 (d, J = 5.6 Hz, 1H), |
| | 7.35 (m, 1H), 7.10-7.20 (m, 2H), 3.95 (m, 1H), 3.85 (s, 3H), 3.00 (m, 2H), |
| | 2.45 (s, 3H), 2.43-2.20 (m, 2H), 1.70-1.90 (m, 4H). |
| 3AK | ¹H NMR (400 MHz, CD₃OD-d₄) δ 8.24 (s, 1H), 8.02 (d, J = 5.6 Hz, 1H), |
| | 7.95 (d, J = 5.6 Hz, 1H), 7.58 (m, 1H), 7.05-7.15 (m, 2H), 4.00 (s, 3H), 3.40 |
| | (d, J = 5.6 Hz, 2H), 3.05 (m, 2H), 2.40 (s, 3H), 2.25 (m, 2H), 1.70-1.90 (m, |
| | 3H), 1.47 (m, 2H). |
| 3AL | ¹H NMR (400 MHz, CD₃OD-d₄) δ 8.28 (s, 1H), 8.06-8.14 (m, 2H), 7.94 (dd, |
| | J = 7.2, 3.0 Hz, 1H), 7.60 (tdd, J = 9.0, 5.7, 1.3 Hz, 1H), 7.05-7.15 (m, 2H), |
| | 4.00 (s, 3H), 3.73 (t, J = 6.7 Hz, 2H), 2.62 (t, J = 6.7 Hz, 2H). |
| 3AM | ¹H NMR (400 MHz, CD₃OD-d₄) δ 8.27 (s, 1H), 8.10 (dd, 1H), 7.95 (d, 1H), |
| | 7.59 (m, 1H), 7.07-7.16 (m, 2H), 4.13 (s, 2H), 3.99 (s, 3H). |
| 3AN | ¹H NMR (400 MHz, CD₃OD-d₄) δ 8.27 (d, J = 3.0 Hz, 1H), 8.06 (d, J = 8.4 |
| | Hz, 1H), 7.97 (dd, J = 7.2, 3.0 Hz, 1H), 7.58 (m, 1H), 7.21-7.33 (m, 2H), 7.16 |
| | (dd, J = 8.3, 6.0 Hz, 1H), 4.00 (s, 3H). |
| 3AO | ¹H NMR (400 MHz, CD₃OD-d₄) δ 3.78 (s, 3H), 4.01 (s, 3H), 6.93 (dd, J = |
| | 9.0, 1.5 Hz, 1H), 7.00 (dd, J = 8.4, 5.7 Hz, 1H), 7.49 (t, J = 9.0 Hz, 1H), 7.88 |
| | (dd, J = 7.2, 3.0 Hz, 1H), 7.99 (d, J = 8.4 Hz, 1H), 8.26 (d, J = 3.0 Hz, 1H). |
| 3AP | ¹H NMR (400 MHz, DMSO-d₆) δ 1.72 (p, J = 6.6 Hz, 2H), 3.40 (m, 2H), 3.50 |
| | (t, J = 6.2 Hz, 2H), 3.87 (s, 3H), 4.53 (bs, 1H), 7.19 (dd, J = 8.3, 6.0 Hz, 1H), |
| | 7.29-7.33 (m, 2H), 8.04 (d, J = 8.3 Hz, 1H), 8.12 (dd, J = 7.3, 3.0 Hz, 1H), |
| | 8.47 (d, J = 3.0 Hz, 1H), 8.53 (t, J = 5.9 Hz, 1H), 10.76 (s, 1H), 14.28 (s, 1H). |
| 3AQ | ¹H NMR (400 MHz, CD₃OD-d₄) δ 3.99 (s, 3H), 7.08 (ddd, J = 8.3, 5.1, 3.1 |
| | Hz, 1H), 7.17 (dd, J = 8.4, 5.9 Hz, 1H), 7.39 (ddd, J = 9.2, 5.9, 3.2 Hz, 1H), |
| | 8.04-8.10 (m, 2H), 8.29 (d, J = 2.9 Hz, 1H). |
| 3AR | ¹H NMR (400 MHz, CD₃OD-d₄) δ 8.81 (d, J = 6.9 Hz, 1H), 8.10 (s, 1H), |
| | 8.01 (d, J = 8.4 Hz, 1H), 7.82 (dt, J = 9.2, 1.2 Hz, 1H), 7.73 (ddd, J = 9.1, 6.8, |
| | 1.2 Hz, 1H), 7.64 (td, J = 8.9, 5.7 Hz, 1H), 7.32 (td, J = 6.9, 1.2 Hz, 1H), 7.18 |
| | (td, J = 8.9, 1.7 Hz, 1H), 6.91 (dd, J = 8.4, 5.7 Hz, 1H), 3.71 (t, J = 6.2 Hz, |
| | 2H), 3.56 (t, J = 6.8 Hz, 2H), 1.88 (p, J = 6.5 Hz, 2H). |
| 3AS | ¹H NMR (400 MHz, CD₃OD-d₄) δ 8.27 (d, J = 3.0 Hz, 1H), 8.15 (d, J = 8.4 |
| | Hz, 1H), 8.05 (dd, J = 7.3, 3.0 Hz, 1H), 7.37 (ddd, J = 9.3, 5.9, 3.1 Hz, 1H), |
| | 7.24 (s, 2H), 7.15 (dd, J = 8.4, 5.9 Hz, 1H), 7.04 (dt, J = 5.9, 3.4 Hz, 1H), 3.99 |
| | (s, 3H). |
| 3AT | ¹H NMR (400 MHz, CD₃OD-d₄) δ 8.27 (s, 1H), 8.07 (d, J = 5.6, 1H), 7.95 |
| | (d, J = 5.6, 1H), 7.60 (m, 1H), 7.15-7.05 (m, 2H), 4.00 (s, 3H), 3.75-3.65 (m, |
| | 4H), 3.58 (d, J = 7.2, 2H), 2.03 (m, 1H). |
| 3AU | ¹H NMR (400 MHz, CD₃OD-d₄) δ 8.64 (d, J = 7.9 Hz, 1H), 8.18 (d, J = 3.0 |
| | Hz, 1H), 8.01 (d, J = 8.4 Hz, 1H), 7.90 (dd, J = 7.8, 3.1 Hz, 1H), 7.34-7.10 |
| | (m, 2H), 6.90 (t, J = 9.1 Hz, 1H), 4.13 (m, 1H), 3.80 (s, 3H), 3.31 (m, 2H), |
| | 2.99 (m, 2H), 1.98 (m, 2H), 1.82 (m, 2H). |
| 3AV | ¹H NMR (400 MHz, CD₃OD-d₄) δ 8.27 (d, J = 3.0 Hz, 1H), 8.08 (d, J = 8.4 |
| | Hz, 1H), 7.94 (dd, J = 7.2, 3.0 Hz, 1H), 7.58 (m, 1H), 7.07-7.15 (m, 2H), 3.99 |
| | (s, 3H), 3.50 (t, J = 6.7 Hz, 2H), 3.31 (d, J = 6.5 Hz, 1H), 1.98 (s, 3H), 1.85 |
| | (m, 2H). |
| 3AW | ¹H NMR (400 MHz, CD₃OD-d₄) δ 8.27 (s, 1H), 8.09 (d, 1H), 7.93 (d, 1H), |
| | 7.60 (m, 1H), 7.05-7.15 (m, 2H), 4.41 (s, 2H), 3.99 (s, 3H). |
| 3AX | ¹H NMR (400 MHz, CD₃OD-d₄) δ 8.27 (d, J = 3.0 Hz, 1H), 8.07 (d, J = 8.4 |
| | Hz, 1H), 7.97 (dd, J = 7.2, 3.0 Hz, 1H), 7.58 (td, J = 7.6, 2.2 Hz, 1H), 7.21- |
| | 7.33 (m, 2H), 7.16 (dd, J = 8.4, 6.0 Hz, 1H), 4.00 (s, 3H), 3.88 (m, 1H), 3.67 |
| | (dd, J = 13.7, 4.9 Hz, 1H), 3.62 (d, J = 5.4 Hz, 2H), 3.49 (dd, J = 13.8, 6.8 Hz, |
| | 1H). |
| 3AY | ¹H NMR (400 MHz, CD₃OD-d₄) δ 8.27 (d, J = 3.0 Hz, 1H), 8.07 (d, J = 8.4 |
| | Hz, 1H), 7.97 (dd, J = 7.2, 3.0 Hz, 1H), 7.58 (td, J = 7.6, 2.2 Hz, 1H), 7.21- |
| | 7.33 (m, 2H), 7.16 (dd, J = 8.4, 6.0 Hz, 1H), 4.00 (s, 3H), 3.88 (m, 1H), 3.67 |
| | (dd, J = 13.7, 4.9 Hz, 1H), 3.62 (d, J = 5.4 Hz, 2H), 3.49 (dd, J = 13.8, 6.8 Hz, |
| | 1H). |
| 3AZ | ¹H NMR (400 MHz, CD₃OD-d₄) δ 8.58 (d, J = 2.8 Hz, 1H), 8.07 (d, J = 8.4 |
| | Hz, 1H), 7.93 (dd, J = 8.1, 2.9 Hz, 1H), 7.60 (td, J = 8.9, 5.7 Hz, 1H), 7.17 |
| | (m, 1H), 7.06 (m, 1H), 3.71 (t, J = 6.2 Hz, 2H), 3.57 (t, J = 6.8 Hz, 2H), 2.82 |
| | (s, 3H), 1.89 (m, 2H). |
| 3BA | ¹H NMR (400 MHz, CD₃OD-d₄) δ 8.58 (d, J = 2.8 Hz, 1H), 8.07 (d, J = 8.3 |
| | Hz, 1H), 7.93 (dd, J = 8.0, 2.8 Hz, 1H), 7.60 (td, J = 8.9, 5.7 Hz, 1H), 7.17 |
| | (td, J = 8.9, 1.8 Hz, 1H), 7.06 (m, 1H), 2.83 (s, 3H). |
| 3BB | ¹H NMR (400 MHz, CD₃OD-d₄) δ 836 (s, 1H), 8.18 (s, 1H), 8.08 (d, 1H), |
| | 7.38 (m, 1H), 7.17 (m, 1H), 7.07 (m, 1H), 3.99 (s, 3H), 3.57 (t, 2H), 3.26 (t, |
| | 2H), 2.95 (s, 6H), 2.10 (m, 2H). |
| 3BC | ¹H NMR (400 MHz, CD₃OD-d₄) δ 9.52 (m, 1H), 8.30 (d, J = 2.5 Hz, 1H), |
| | 8.09 (d, J = 2.5 Hz, 1H), 7.75 (m, 1H), 7.65 (m, 1H), 7.53 (m, 1H), 7.25 (m, |
| | 1H), 3.99 (s, 3H), 3.69 (m, 2H), 3.56 (m, 2H), 1.88 (m, 2H). |
| 3BD | ¹H NMR (400 MHz, CD₃OD-d₄) δ 9.51 (m, 1H), 8.30 (d, J = 2.5 Hz, 1H), |
| | 8.09 (d, J = 2.5 Hz, 1H), 7.75 (s, 1H), 7.65 (m, 1H), 7.53 (m, 1H), 7.25 (br t, |
| | J = 8.0 Hz, 1H), 3.99 (s, 3H). |
| 3BE | ¹H NMR (400 MHz, CD₃OD-d₄) δ 8.28 (s, 1H), 8.09 (s, 1H), 7.93 (d, 1H), |
| | 7.58 (m, 1H), 7.06-7.11 (m, 2H), 3.98 (s, 3H), 3.77 (m, 1H), 3.48-3.71 (m, |
| | 4H), 1.95 (m, 1H), 1.70 (m, 1H). |
| 3BF | ¹H NMR (400 MHz, CD₃OD-d₄) δ 8.28 (s, 1H), 8.09 (s, 1H), 7.93 (d, 1H), |
| | 7.58 (m, 1H), 7.06-7.11 (m, 2H), 3.98 (s, 3H), 3.77 (m, 1H), 3.48-3.71 (m, |
| | 4H), 1.95 (m, 1H), 1.70 (m, 1H). |
| 3BG | ¹H NMR (400 MHz, CD₃OD-d₄) δ 8.27 (d, J = 3.0 Hz, 1H), 8.06 (d, J = 8.3 |
| | Hz, 1H), 7.97 (dd, J = 7.3, 3.0 Hz, 1H), 7.57 (m, 1H), 7.21-7.34 (m, 2H), 7.15 |
| | (dd, J = 8.3, 6.0 Hz, 1H), 4.00 (s, 3H), 3.76 (m, 1H), 3.50-3.70 (m, 4H), 1.93 |
| | (m, 1H), 1.70 (m, 1H). |
| 3BH | ¹H NMR (400 MHz, CD₃OD-d₄) δ 8.27 (d, J = 3.0 Hz, 1H), 8.06 (d, J = 8.3 |
| | Hz, 1H), 7.97 (dd, J = 7.3, 3.0 Hz, 1H), 7.57 (m, 1H), 7.21-7.34 (m, 2H), 7.15 |
| | (dd, J = 8.3, 6.0 Hz, 1H), 4.00 (s, 3H), 3.76 (m, 1H), 3.50-3.70 (m, 4H), 1.93 |
| | (m, 1H), 1.70 (m, 1H). |
| 3BI | ¹H NMR (400 MHz, CD₃OD-d₄) δ 8.36 (dd, J = 5.0, 1.9 Hz, 1H), 8.03-8.14 |
| | (m, 2H), 7.58 (m, 1H), 7.02-7.14 (m, 3H), 4.02 (s, 3H), 3.71 (t, J = 6.2 Hz, |
| | 2H), 3.57 (t, J = 6.8 Hz, 2H), 1.89 (m, 2H). |
| 3BJ | ¹H NMR (400 MHz, CD₃OD-d₄) δ 8.36 (dd, J = 5.0, 1.9 Hz, 1H), 8.03-8.14 |
| | (m, 2H), 7.58 (m, 1H), 7.02-7.14 (m, 3H), 4.02 (s, 3H). |
| 3BK | ¹H NMR (400 MHz, CD₃OD-d₄) δ 8.21 (d, J = 3.0 Hz, 1H), 8.01-8.10 (m, |
| | 2H), 7.31 (m, 1H), 7.19 (dd, J = 7.9, 3.8 Hz, 1H), 6.88 (m, 1H), 3.95 (s, 3H), |
| | 3.52 (t, J = 6.8 Hz, 2H), 2.74 (t, J = 7.7 Hz, 2H), 2.51 (s, 6H), 1.95 (m, 2H). |
| 3BL | ¹H NMR (400 MHz, CD₃OD-d₄) δ 2.35 (dt, J = 14.0, 7.1 Hz, 1H), 2.66 (s, |
| | 1H), 3.04 (s, 3H), 3.25-3.14 (m, 1H), 3.63-3.43 (m, 1H), 4.20-3.77 (m, |
| | 5H), 4.72 (s, 1H), 7.08 (ddd, J = 8.6, 5.1, 2.9 Hz, 1H), 7.24-7.17 (m, 1H), |
| | 7.39 (dq, J = 9.2, 3.2 Hz, 1H), 8.09 (d, J = 8.4 Hz, 1H), 8.19 (d, J = 2.6 Hz, |
| | 1H), 8.36 (d, J = 2.8 Hz, 1H). |
| 3BM | ¹H NMR (400 MHz, CD₃OD-d₄) δ 2.35 (dt, J = 14.0, 7.1 Hz, 1H), 2.66 (s, |
| | 1H), 3.04 (s, 3H), 3.25-3.14 (m, 1H), 3.63-3.43 (m, 1H), 4.20-3.77 (m, |
| | 5H), 4.72 (s, 1H), 7.08 (ddd, J = 8.6, 5.1, 2.9 Hz, 1H), 7.24-7.17 (m, 1H), |
| | 7.39 (dq, J = 9.2, 3.2 Hz, 1H), 8.09 (d, J = 8.4 Hz, 1H), 8.19 (d, J = 2.6 Hz, |
| | 1H), 8.36 (d, J = 2.8 Hz, 1H). |
| 3BN | ¹H NMR (400 MHz, CD₃OD-d₄) δ 9.21 (s, 1H), 8.71 (s, 1H), 8.27 (s, 1H), |
| | 8.15 (d, 1H), 7.96 (m, 1H), 7.60 (m, 1H), 7.09-7.18 (m, 2H), 3.99 (s, 3H). |
| 3BO | ¹H NMR (400 MHz, CD₃OD-d₄) δ 8.28 (d, J = 3.0 Hz, 1H), 8.14 (d, J = 8.4 |
| | Hz, 1H), 8.03 (s, 2H), 7.95 (dd, J = 7.2, 3.0 Hz, 1H), 7.60 (m, 1H), 7.13 (td, |
| | J = 8.1, 7.3, 1.8 Hz, 2H), 4.00 (s, 3H). |
| 3BP | ¹H NMR (400 MHz, CD₃OD-d₄) δ 8.35 (s, 1H), 8.27 (s, 1H), 8.27 (d, 1H), |
| | 7.96 (d, 1H), 7.82 (m, 1H), 7.58 (m, 1H), 7.09-7.17 (m, 2H), 6.63 (d, 1H), |
| | 4.00 (s, 3H), 3.65 (s, 3H). |
| 3BQ | ¹H NMR (400 MHz, CD₃OD-d₄) δ 8.36 (s, 1H), 8.13 (d, 1H), 8.08 (s, 1H), |
| | 8.07 (s, 1H), 7.74 (s, 1H), 7.62 (m, 1H), 7.11-7.17 (m, 2H), 4.04 (s, 3H), 3.93 |
| | (s, 3H). |
| 3BR | ¹H NMR (400 MHz, CD₃OD-d₄) δ 8.31 (d, J = 3.0 Hz, 1H), 8.11-8.19 (m, |
| | 2H), 7.81 (dd, J = 8.6, 7.5 Hz, 1H), 7.69 (dd, J = 8.6, 1.3 Hz, 1H), 7.22 (dd, |
| | J = 8.4, 5.8 Hz, 1H), 3.95 (s, 3H). |
| 3BS | ¹H NMR (400 MHz, CD₃OD-d₄) δ 8.23 (d, J = 3.0 Hz, 1H), 8.02-8.10 (m, |
| | 2H), 7.33 (m, 1H), 7.18 (m, 1H), 6.95 (m, 1H), 4.67 (m, 1H), 3.96 (s, 3H), |
| | 3.04 (m, 2H), 2.87 (dd, J = 10.8, 4.5 Hz, 1H), 2.72 (m, 1H), 2.54 (s, 3H), 2.49 |
| | (m, 1H), 1.98 (dd, J = 13.1, 6.3 Hz, 1H). |
| 3BT | ¹H NMR (400 MHz, CD₃OD-d₄) δ 8.23 (d, J = 3.0 Hz, 1H), 8.02-8.10 (m, |
| | 2H), 7.33 (m, 1H), 7.18 (m, 1H), 6.95 (m, 1H), 4.67 (m, 1H), 3.96 (s, 3H), |
| | 3.04 (m, 2H), 2.87 (dd, J = 10.8, 4.5 Hz, 1H), 2.72 (m, 1H), 2.54 (s, 3H), 2.49 |
| | (m, 1H), 1.98 (dd, J = 13.1, 6.3 Hz, 1H). |
| 3BU | ¹H NMR (400 MHz, CD₃OD-d₄) δ 8.74 (s, 1H), 8.41 (s, 1H), 8.09 (d, 1H), |
| | 7.61 (m, 1H), 7.11-7.17 (m, 2H), 4.10 (s, 3H), 3.59 (t, 2H), 3.26 (t, 2H), 2.95 |
| | (s, 6H), 2.09 (m, 2H). |
| 3BV | ¹H NMR (400 MHz, CD₃OD-d₄) δ 8.75 (d, J = 2.2 Hz, 1H), 8.52 (d, J = 2.2 |
| | Hz, 1H), 8.15 (d, J = 8.4 Hz, 1H), 7.40 (m, 1H), 7.25 (s, 2H), 7.06-7.19 (m, |
| | 2H), 4.09 (s, 3H). |
| 3BW | ¹H NMR (400 MHz, DMSO-d₆) δ 14.74 (bs, 1H), 10.50 (bs, 1H), 9.91 (s, |
| | 1H), 8.40-8.52 (m, 2H), 8.12 (d, J = 5.6, 1H), 8.01 (d, J = 5.6, 1H), 7.42-7.55 |
| | (m, 2H), 7.20-7.30 (m, 2H), 6.37 (t, J = 5.6, 1H), 3.89 (s, 3H), 3.60 (s, 3H). |
| 3BX | ¹H NMR (400 MHz, DMSO-d₆) δ 14.56 (bs, 1H), 10.68 (s, 1H), 10.46 (bs, |
| | 1H), 8.94 (s, 1H), 8.46 (s, 1H), 8.17 (d, J = 8.4, 1H), 8.11 (d, J = 8.4, 1H), |
| | 8.02 (d, J = 6.8, 1H), 7.46 (m, 1H), 7.19-7.29 (m, 3H), 3.90 (s, 3H), 2.46 (s, |
| | 3H). |
| 3BY | ¹H NMR (400 MHz, CD₃OD-d₄) δ 8.13 (m, 1H), 7.37 (ddd, J = 9.1, 5.9, 3.1 |
| | Hz, 1H), 7.22 (s, 2H), 7.07-7.17 (m, 2H), 2.63 (s, 3H), 2.46 (s, 3H). |
| 3BZ | ¹H NMR (400 MHz, CD₃OD-d₄) δ 9.34 (m, 1H), 8.01 (d, J = 8.6 Hz, 1H), |
| | 7.71 (s, 2H), 7.40 (m, 1H), 7.33 (m, 1H), 7.09 (m, 1H), 3.99 ppm (s, 3H) |
| 3CA | ¹H NMR (400 MHz, CD₃OD-d₄) δ 8.30 (s, 1H), 8.12-8.17 (m, 2H), 7.82 (m, |
| | 1H), 7.70 (d, J = 5.6, 1H), 7.22 (m, 1H), 3.95 (s, 3H). 3.70 (t, J = 4.8 Hz, 2H), |
| | 3.59 (t, J = 4.8 Hz, 2H), 1.85-1.95 (m, 2H). |
| 3CB | ¹H NMR (400 MHz, CD₃OD-d₄) δ 8.28 (d, J = 2.8 Hz, 1H), 8.18 (d, J = 8.4 |
| | Hz, 1H), 8.07 (m, 1H), 7.96 (m, 1H), 7.71 (d, J = 4.4 Hz, 1H), 7.25 (s, 2H), |
| | 7.18 (m, 1H), 3.95 (s, 3H). |
| 3CC | ¹H NMR (400 MHz, CD₃OD-d₄) δ 8.55 (d, J = 2.4 Hz, 1H), 8.16 (d, J = 8.4 |
| | Hz, 1H), 8.07 (m, 1H), 7.90 (m, 1H), 7.68 (d, J = 4.4 Hz, 1H), 7.25 (s, 2H), |
| | 7.15 (m, 1H), 2.87 (s, 3H). |
| 3CD | ¹H NMR (400 MHz, CD₃OD-d₄) δ 8.58 (d, J = 2.8 Hz, 1H), 8.03-8.10 (m, |
| | 2H), 7.38 (ddd, J = 9.1, 5.9, 3.2 Hz, 1H), 7.10-7.19 (m, 2H), 3.57 (t, J = 6.5 |
| | Hz, 2H), 3.26 (m, 2H), 2.95 (s, 6H), 2.85 (s, 3H), 2.09 (m, 2H). |
| 3CE | ¹H NMR (400 MHz, CD₃OD-d₄) δ 8.57 (d, J = 2.1 Hz, 1H), 8.19 (d, J = 2.2 |
| | Hz, 1H), 8.02 (d, J = 8.4 Hz, 1H), 7.45 (m, 1H), 7.17-7.27 (m, 2H), 4.05 (s, |
| | 3H), 3.68 (t, J = 6.8 Hz, 2H), 3.17 (m, 2H), 2.85 (s, 6H), 2.05 (m, 2H). |
| 3CF | ¹H NMR (400 MHz, CD₃OD-d₄) δ 8.33 (d, J = 2.5 Hz, 1H), 8.18 (d, J = 2.6 |
| | Hz, 1H), 8.07 (d, J = 8.4 Hz, 1H), 7.34 (m, 1H), 7.18 (dd, J = 8.4, 5.9 Hz, |
| | 1H), 7.00 (m, 1H), 3.98 (s, 3H). |
| 3CG | ¹H NMR (400 MHz, CD₃OD-d₄) δ 8.58 (d, J = 2.8 Hz, 1H), 8.00-8.10 (m, |
| | 2H), 7.38 (m, 1H), 7.11-7.16 (m, 2H), 2.85 (s, 3H). |
| 3CH | ¹H NMR (400 MHz, DMSO-d₆) δ 10.69 (br s, 1H), 8.16 (d, J = 8.61 Hz, 1H), |
| | 7.71 (s, 2H), 7.52-7.66 (m, 2H), 7.38 (td, J = 8.80, 5.87 Hz, 1H), 7.17-7.30 |
| | (m, 2H), 5.73 (s, 1H), 4.59 (s, 2H). |
| 3CK | ¹H NMR (400 MHz, CD₃OD-d₄) δ 8.29 (s, 1H), 8.13 (d, J = 5.6, 1H), 7.95 |
| | (d, J = 5.6, 1H), 7.60 (m, 1H), 7.10-7.18 (m, 2H), 4.00 (s, 3H), 2.69 (s, 3H). |
| 3CL | ¹H NMR (400 MHz, CD₃OD-d₄) δ 8.64 (s, 1H), 8.40 (s, 1H), 8.02 (d, J = 8.4 |
| | Hz, 1H), 7.66 (m, 1H), 7.12-7.26 (m, 2H), 4.05 (s, 3H), 3.71 (t, J = 6.8 Hz, |
| | 2H), 3.64 (t, J = 6.0 Hz, 2H), 1.84 (m, 2H). |
| 3CM | ¹H NMR (400 MHz, CD₃OD-d₄) δ 8.64 (s, 1H), 8.38 (s, 1H), 8.02 (d, J = 8.4 |
| | Hz, 1H), 7.69 (m, 1H), 7.23 (m, 1H), 7.13 (m, 1H), 4.05 (s, 3H), 3.68 (m, 2H), |
| | 3.13-3.20 (m, 2H), 2.84 (s, 6H), 2.06 (m, 2H). |
| 3CN | ¹H NMR (400 MHz, CD₃OD-d₄) δ 8.32 (d, J = 2.3 Hz, 1H), 8.13 (d, J = 2.4 |
| | Hz, 1H), 8.07 (d, J = 8.4 Hz, 1H), 7.59 (m, 1H), 7.04-7.10 (m, 2H), 4.58 (s, |
| | 2H), 4.02 (s, 3H), 3.71 (t, J = 6.2 Hz, 2H), 3.57 (t, J = 6.8 Hz, 2H), 1.89 (m, |
| | 2H). |
| 3CO | ¹H NMR (400 MHz, CD₃OD-d₄) δ 8.02-8.06 (m, 2H), 7.49 (m, 1H), 7.19 (d, |
| | J = 8.0 Hz, 1H), 7.03 (m, 2H), 3.51 (m, 2H), 2.84 (s, 3H), 2.64 (m, 2H), 2.55 |
| | (s, 3H), 2.43 (s, 6H), 1.92 (m, 2H). |
| 3CP | ¹H NMR (400 MHz, CD₃OD-d₄) δ 7.99-8.09 (m, 2H), 7.57 (m, 1H), 7.22 (d, |
| | J = 8.0 Hz, 1H), 7.17 (m, 1H), 7.07 (m, 1H), 3.71 (m, 2H), 3.57 (m, 2H), 2.82 |
| | (s, 3H), 2.57 (s, 3H), 1.90 (m, 2H). |
| 3CQ | ¹H NMR (400 MHz, CD₃OD-d₄) δ 8.37 (s, 1H), 8.14 (d, 1H), 8.08 (d, 1H), |
| | 7.99 (s, 2H), 7.74 (m, 1H), 7.62 (m, 1H), 7.11-7.18 (m, 2H), 4.01 (s, 3H). |
| 3CR | ¹H NMR (400 MHz, CD₃OD-d₄) δ 8.50 (s, 1H), 8.35 (d, J = 2.5 Hz, 1H), |
| | 8.15 (d, J = 8.4 Hz, 1H), 8.09 (d, J = 2.6 Hz, 1H), 8.00 (s, 1H), 7.60 (m, 1H), |
| | 7.49 (t, 1H), 7.10-7.18 (m, 2H), 4.01 (s, 3H). |
| 3CS | ¹H NMR (400 MHz, CD₃OD-d₄) δ 8.34 (d, J = 2.6 Hz, 1H), 8.06-8.15 (m, |
| | 2H), 7.71 (m, 1H), 7.58 (m, 1H), 7.10-7.17 (m, 2H), 4.00 (s, 3H), 2.34 (s, 3H). |
| 3CT | ¹H NMR (400 MHz, CD₃OD-d₄) δ 8.37 (d, J = 2.6 Hz, 1H), 8.12-8.20 (m, |
| | 2H), 8.09 (d, J = 2.6 Hz, 1H), 7.79 (s, 1H), 7.60 (m, 1H), 7.10-7.16 (m, 2H), |
| | 4.26 (t, J = 5.4 Hz, 2H), 4.01 (s, 3H), 3.93 (t, J = 5.4 Hz, 2H). |
| 3CU | ¹H NMR (400 MHz, CD₃OD-d₄) δ 8.37 (s, 1H), 8.16 (d, J = 5.6, 1H), 8.10 (s, |
| | 1H), 7.60 (m, 1H), 7.05-7.20 (m, 2H), 4.01 (s, 3H). |
| 3CV | ¹H NMR (400 MHz, CD₃OD-d₄) δ 9.21 (s, 1H), 8.71 (s, 1H), 8.36 (s, 1H), |
| | 8.13 (m, 1H), 8.08 (s, 1H), 7.60 (m, 1H), 7.10-7.17 (m, 2H), 4.00 (s, 3H). |
| 3CW | ¹H NMR (400 MHz, CD₃OD-d₄) δ 8.29 (s, 1H), 8.05 (d, J = 7.2, 1H), 7.95 |
| | (d, J = 5.6, 1H), 7.60 (m, 1H), 7.05-7.17 (m, 2H), 4.00 (s, 3H). |
| 3CX | ¹H NMR (400 MHz, CD₃OD-d₄) δ 8.89 (s, 1H), 8.78 (s, 1H), 8.16 (d, J = 8.4 |
| | Hz, 1H), 7.61 (m, 1H), 7.24 (s, 2H), 7.06-7.16 (m, 2H), 4.10 (s, 3H). |
| 3CY | ¹H NMR (400 MHz, CD₃OD-d₄) δ 8.68 (d, J = 2.2 Hz, 1H), 8.48 (d, J = 2.2 |
| | Hz, 1H), 8.07 (d, J = 8.4 Hz, 1H), 7.30 (m, 1H), 7.19 (dd, J = 8.3, 6.0 Hz, |
| | 1H), 6.92 (m, 1H), 4.05 (s, 3H). |
| 3CZ | ¹H NMR (400 MHz, CD₃OD-d₄) δ 8.35 (s, 1H), 8.06-8.09 (m, 2H), 7.58 (m, |
| | 1H), 7.07-7.15 (m, 2H), 3.99 (s, 3H), 3.33 (s, 3H). |
| 3DA | ¹H NMR (400 MHz, CD₃OD-d₄) δ 8.36 (d, J = 2.6 Hz, 1H), 8.15 (d, 1H), |
| | 8.09 (s, 1H), 7.78 (d, J = 2.1 Hz, 1H), 7.71 (dt, J = 10.9, 2.1 Hz, 1H), 7.60 (m, |
| | 1H), 7.08-7.19 (m, 2H), 6.98 (m, 1H), 4.01 (s, 3H). |
| 3DB | ¹H NMR (400 MHz, CD₃OD-d₄) δ 8.31 (s, 1H), 8.13 (d, 1H), 8.06 (d, 1H), |
| | 7.56 (m, 1H), 7.05-7.11 (m, 2H), 4.58 (s, 2H), 4.01 (s, 3H), 3.50 (t, 2H), 2.57 |
| | (t, 2H), 2.38 (s, 6H), 1.92 (m, 2H). |
| 3DC | ¹H NMR (400 MHz, CD₃OD-d₄) δ 8.33 (s, 1H), 8.14 (s, 1H), 8.03 (d, 1H), |
| | 7.71 (s, 2H), 7.59 (m, 1H), 7.26 (m, 1H), 7.12 (m, 1H), 4.58 (s, 2H), 4.03 (s, |
| | 3H). |
| 3DD | ¹H NMR (400 MHz, CD₃OD-d₄) δ 8.15 (d, J = 8.4 Hz, 1H), 7.99 (s, 1H), |
| | 7.59 (m, 1H), 7.25 (s, 2H), 7.02-7.17 (m, 2H), 3.99 (s, 3H), 2.58 (s, 3H). |
| 3DE | ¹H NMR (400 MHz, CD₃OD-d₄) δ 8.32 (s, 1H), 8.13 (s, 1H), 8.06 (d, 1H), |
| | 7.58 (m, 1H), 7.07-7.11 (m, 2H), 4.58 (s, 2H), 4.02 (s, 3H). |
| 3DF | ¹H NMR (400 MHz, CD₃OD-d₄) δ 8.36 (d, J = 2.4, 1H), 8.14-8.19 (m, 2H), |
| | 8.08 (s, 1H), 7.62 (m, 1H), 7.12-7.18 (m, 2H), 4.01 (s, 3H). |
| 3DG | ¹H NMR (400 MHz, CD₃OD-d₄) δ 9.21 (s, 1H), 8.71 (s, 1H), 8.13 (d, J = 8.4 |
| | Hz, 1H), 8.02 (d, J = 8.2 Hz, 1H), 7.57 (m, 1H), 7.22 (d, J = 8.1 Hz, 1H), |
| | 7.03-7.17 (m, 2H), 2.83 (s, 3H), 2.57 (s, 3H). |
| 3DH | ¹H NMR (400 MHz, CD₃OD-d₄) δ 8.06-8.13 (m, 2H), 8.02 (d, J = 8.2 Hz, |
| | 1H), 7.74 (s, 1H), 7.56 (m, 1H), 7.21 (d, J = 8.1 Hz, 1H), 7.01-7.15 (m, 2H), |
| | 3.93 (s, 3H), 2.83 (s, 3H), 2.57 (s, 3H). |
| 3DI | ¹H NMR (400 MHz, CD₃OD-d₄) δ 8.13 (s, 1H), 8.12 (d, J = 8.4 Hz, 1H), |
| | 8.02 (d, J = 8.2 Hz, 1H), 7.84 (s, 1H), 7.57 (m, 1H), 7.22 (d, J = 8.2 Hz, 1H), |
| | 7.01-7.16 (m, 2H), 2.83 (s, 3H), 2.57 (s, 3H). |
| 3DJ | ¹H NMR (400 MHz, CD₃OD-d₄) δ 8.75 (s, 1H), 8.42 (s, 1H), 8.14 (d, 1H), |
| | 7.61 (m, 1H), 7.25 (s, 2H), 7.15 (m, 1H), 7.08 (m, 1H), 4.10 (s, 3H). |
| 3DK | ¹H NMR (400 MHz, CD₃OD-d₄) δ 8.31 (s, 1H), 8.15 (d, J = 8.4 Hz, 1H), |
| | 7.54 (m, 1H), 7.25 (s, 2H), 7.04-7.16 (m, 2H), 4.06 (s, 3H), 2.71 (s, 3H). |
| 3DL | ¹H NMR (400 MHz, CD₃OD-d₄) δ 8.02-8.05 (m, 2H), 7.53 (m, 1H), 7.20 (d, |
| | J = 8.2 Hz, 1H), 7.00-7.12 (m, 2H), 2.84 (s, 3H), 2.56 (s, 3H). |
| 3DM | ¹H NMR (400 MHz, DMSO-d₆) δ 8.32 (s, 1H), 8.23 (d, J = 8.41 Hz, 1H), 7.83 |
| | (dd, J = 7.92, 3.03 Hz, 1H), 7.71 (s, 2H), 7.62 (br dd, J = 9.00, 2.93 Hz, 1H), |
| | 7.43 (br dd, J = 7.73, 5.97 Hz, 1H), 5.73 (s, 1H), 4.60 (s, 2H). |
| 3DN | ¹H NMR (400 MHz, CD₃OD-d₄) δ 8.08 (d, 1H), 7.99 (s, 1H), 7.58 (m, 1H), |
| | 7.09-7.15 (m, 2H), 3.98 (s, 3H), 3.58 (t, 2H), 3.27 (t, 2H), 2.95 (s, 6H), 2.56 |
| | (s, 3H), 2.10 (m, 2H). |
| 3DO | ¹H NMR (400 MHz, CD₃OD-d₄) δ 8.07 (d, 1H), 7.99 (s, 1H), 7.58 (m, 1H), |
| | 7.07-7.14 (m, 2H), 3.98 (s, 3H), 3.70 (t, 2H), 3.59 (t, 2H), 2.57 (s, 3H), 1.89 |
| | (m, 2H). |
| 3DP | ¹H NMR (400 MHz, CD₃OD-d₄) δ 8.72 (s, 1H), 8.50 (s, 1H), 8.07 (d, 1H), |
| | 7.37 (m, 1H), 7.18 (m, 1H), 7.02 (m, 1H), 4.07 (s, 3H), 3.71 (t, 2H), 3.58 (t, |
| | 2H), 1.89 (m, 2H). |
| 3DQ | ¹H NMR (400 MHz, CD₃OD-d₄) δ 9.21 (s, 1H), 8.71 (s, 1H), 8.56 (s, 1H), |
| | 8.13 (d, 1H), 7.94 (dd, 1H), 7.60 (m, 1H), 7.09-7.19 (m, 2H), 2.83 (s, 3H). |
| 3DR | ¹H NMR (400 MHz, CD₃OD-d₄) δ 8.57 (s, 1H), 8.12 (d, 1H), 8.07 (s, 1H), |
| | 7.93 (m, 1H), 7.73 (s, 1H), 7.59 (m, 1H), 7.07-7.19 (m, 2H), 3.93 (s, 3H), 2.83 |
| | (s, 3H). |
| 3DS | ¹H NMR (400 MHz, CD₃OD-d₄) δ 8.58 (s, 1H), 8.12 (d, 1H), 7.99 (s, 1H), |
| | 7.94 (m, 1H), 7.61 (m, 1H), 7.45 (bs, 1H), 7.17 (m, 1H), 7.11 (m, 1H), 2.82 (s, |
| | 3H). |

### Example 38—Synthesis of N-[2,4-difluoro-3-[7-fluoro-3-(1H-imidazol-2-yl)-1H-pyrazolo[4,3-c]pyridin-6-yl]phenyl]-5-fluoro-2-methyl-benzenesulfonamide

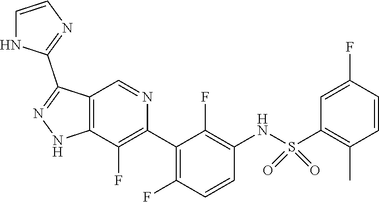 [SMILES: Cc1ccc(F)cc1S(=O)(=O)Nc1ccc(F)c(-c2ncc3c(-c4ncc[nH]4)n[nH]c3c2F)c1F]

### Part I—Synthesis of N-(3-bromo-2,4-difluoro-phenyl)-5-fluoro-2-methyl-benzenesulfonamide

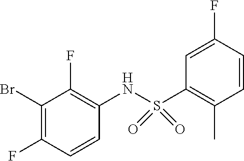 [SMILES: Cc1ccc(F)cc1S(=O)(=O)Nc1ccc(F)c(Br)c1F]

A mixture of 5-fluoro-2-methyl-benzenesulfonyl chloride (2.0062 g, 9.6154 mmol) and 3-bromo-2,4-difluoro-aniline (2 g, 9.6154 mmol) in pyridine (20 mL) was stirred at room temperature overnight. Methanol (20 mL) was added and the mixture was stirred at room temperature for 10 minutes. The reaction mixture was concentrated under reduced pressure the residue was partitioned between water and dichloromethane, aqueous phase was extracted with dichloromethane (3×30 mL) and the combined organics dried (MgSO₄) and concentrated. The residue was purified by silica gel chromatography eluting with 0 to 20% ethyl acetate/hexanes to yield N-(3-bromo-2,4-difluoro-phenyl)-5-fluoro-2-methyl-benzenesulfonamide (3.7 g, quantitative yield) as a beige solid.

### Part II—Synthesis of N-(3-bromo-2,4-difluoro-phenyl)-5-fluoro-2-methyl-N-(2-trimethylsilylethoxymethyl)benzenesulfonamide

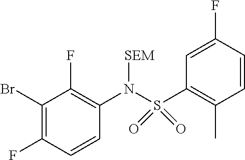 [SMILES: Cc1ccc(F)cc1S(=O)(=O)N(COCC[Si](C)(C)C)c1ccc(F)c(Br)c1F]

A solution of N-(3-bromo-2,4-difluoro-phenyl)-5-fluoro-2-methyl-benzenesulfonamide (0.69 g, 1.81 mmol) in THE (20 mL), and the solution was cooled in an ice bath. Sodium hydride 60% in oil (0.076 g, 2 mmol) was added portion-wise with stirring, and the mixture was stirred in the ice bath for 1 h after the addition of sodium hydride was complete. Then was added drop-wise 2-(trimethylsilyl)ethoxymethyl chloride (0.32 g, 1.9 mmol) and the reaction was allowed to reach room temperature and stirred overnight. The mixture was quenched with NH₄Cl saturated solution and the product was extracted with dichloromethane (3×50 mL). The combined organics were dried with MgSO₄ and concentrated. The residue was purified by silica gel chromatography eluting with 0 to 20% hexanes/ethyl acetate to yield N-(3-bromo-2,4-difluoro-phenyl)-5-fluoro-2-methyl-N-(2-trimethylsilylethoxymethyl)benzenesulfonamide (0.86 g, 93% yield) as a colorless liquid.

### Part III—Synthesis of N-(2,4-difluoro-3-tributylstannyl-phenyl)-5-fluoro-2-methyl-N-(2-trimethylsilylethoxymethyl)benzenesulfonamide

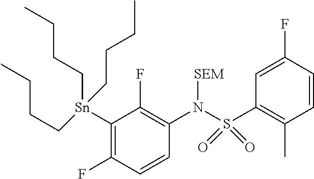 [SMILES: CCC[CH2][Sn]([CH2]CCC)([CH2]CCC)[c]1c(F)ccc(N(COCC[Si](C)(C)C)S(=O)(=O)c2cc(F)ccc2C)c1F]

A solution of N-(3-bromo-2,4-difluoro-phenyl)-5-fluoro-2-methyl-N-(2-trimethylsilylethoxymethyl)benzenesulfonamide (0.1 g, 0.2 mmol) in THE (2 mL) was cooled in an ice/methanol bath (about −10° C.) and then was added drop-wise with stirring isopropylmagnesium chloride 2 M solution in THE (0.11 mL, 0.23 mmol). After the addition was complete, the mixture was stirred in the ice bath for 10 min. Then the mixture was cooled in a dry ice/acetone bath and was added drop-wise tributyltin chloride (0.07 g, 0.2 mmol) and the reaction was allowed to reach room temperature with stirring overnight. The mixture was quenched with NH₄Cl saturated solution and the product was extracted with dichloromethane (3×50 mL), combined organics were dried with MgSO₄ and concentrated. The residue was purified by silica gel chromatography eluting with 0 to 10% hexanes/ethyl acetate to yield N-(2,4-difluoro-3-tributylstannyl-phenyl)-5-fluoro-2-methyl-N-(2-trimethylsilylethoxymethyl)benzenesulfonamide (0.072 g, 51% yield) as a colorless dense liquid.

### Part IV—Synthesis of N-[2,4-difluoro-3-[7-fluoro-3-(1H-imidazol-2-yl)-1H-pyrazolo[4,3-c]pyridin-6-yl]phenyl]-5-fluoro-2-methyl-benzenesulfonamide

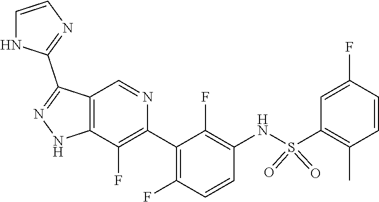 [SMILES: Cc1ccc(F)cc1S(=O)(=O)Nc1ccc(F)c(-c2ncc3c(-c4ncc[nH]4)n[nH]c3c2F)c1F]

A small microwave vial was charged under nitrogen with a solution of 2-[[6-chloro-7-fluoro-3-[1-(2-trimethylsilylethoxymethyl)imidazol-2-yl]pyrazolo[4,3-c]pyridin-1-yl]methoxy]ethyl-trimethyl-silane (0.045 g, 0.09 mmol), N-(2,4-difluoro-3-tributylstannyl-phenyl)-5-fluoro-2-methyl-N-(2-trimethylsilylethoxymethyl)benzenesulfonamide (0.072 g, 0.1 mmol) and copper(I) iodide (0.004 g, 0.02 mmol) in 1,4-dioxane (1 mL) and the mixture was evacuated and back filled with nitrogen and then was added bis(triphenylphosphine)palladium(II) dichloride (0.007 g, 0.01 mmol) then nitrogen was bubbled into the solution while the flask was immersed in an ultrasound bath for ten minutes. Then the reaction mixture was heated under microwaves at 120° C. for 30 min. LCMS showed mostly starting material. Added one full equivalent of the palladium catalyst and CuI and the mixture heated under microwaves at 110° C. for 2 h and then at 150° C. for 3 h. The mixture was concentrated and used in the next step without further treatment.

To a solution of the crude, from the previous step, in dichloromethane (2 mL) was added trifluoroacetic acid (1 mL) and the mixture was stirred at room temperature for 4 h. The mixture was concentrated, the residue was diluted with MeOH containing 10% NH₄OH, and the solution was stirred at room temperature for 2 h. The mixture was concentrated and the residue was purified by preparative HPLC to give N-[2,4-difluoro-3-[7-fluoro-3-(1H-imidazol-2-yl)-1H-pyrazolo[4,3-c]pyridin-6-yl]phenyl]-5-fluoro-2-methyl-benzenesulfonamide; 2,2,2-trifluoroacetic acid solvate (0.9 mg, 1.6% yield) as a white foam.

### Example 39—Preparation of Additional (Aza)Indazolyl-Aryl Sulfonamide and Related Compounds

Compounds in Table 7 were prepared based on experimental procedures described in Examples 1-38 and in the Detailed Description.

TABLE 7

| | | Molecular |
|---|---|---|
| Compd | | Weight |
| No. | Structure | (g/mol) |
| 4A | 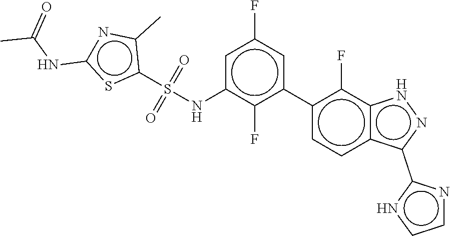 | 547.53 |
| 4B | 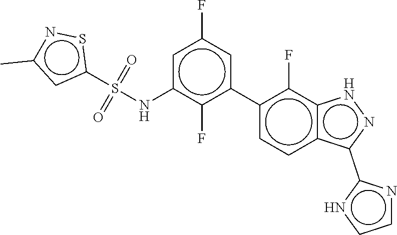 | 490.48 |
| 4C | 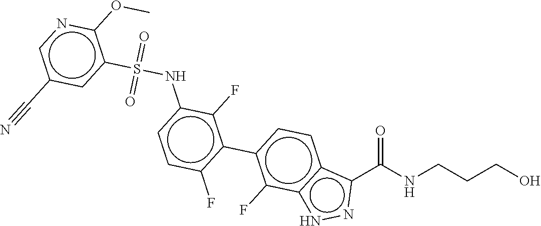 | 560.51 |
| 4D | 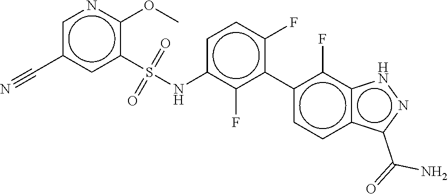 | 502.43 |
| 4E | 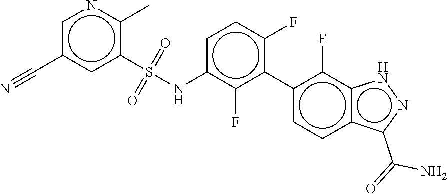 | 486.43 |
| 4F | 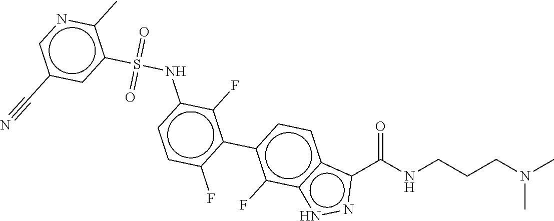 | 571.57 |
| 4G | 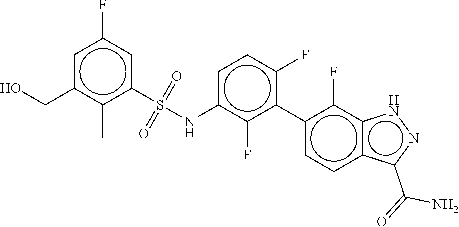 | 508.45 |
| 4H | 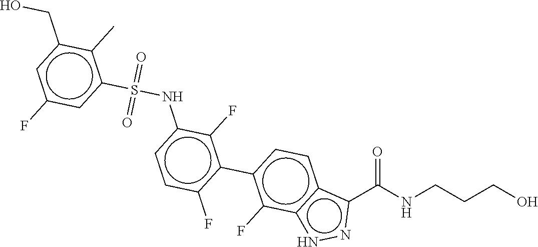 | 566.52 |
| 4I | 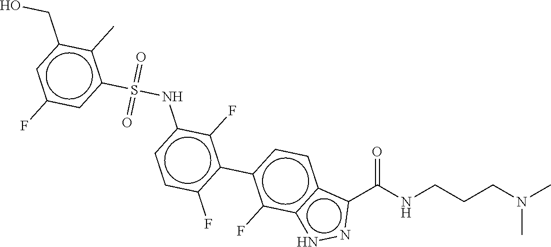 | 593.59 |
| 4J | 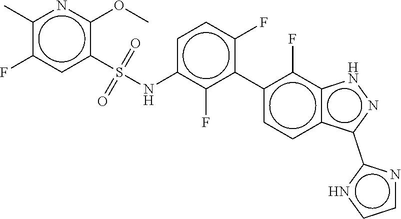 | 532.47 |
| 4K | 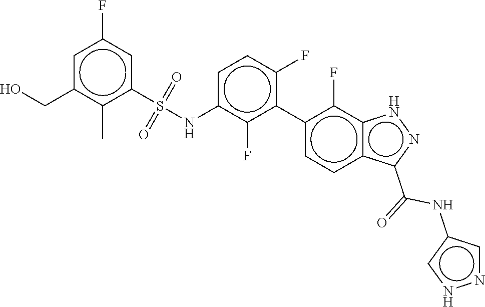 | 574.51 |
| 4L | 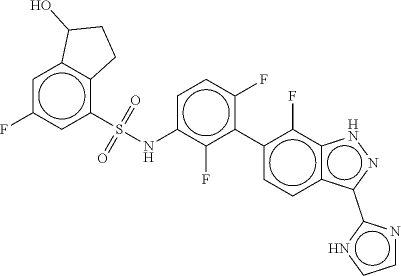 | 543.49 |
| 4M | 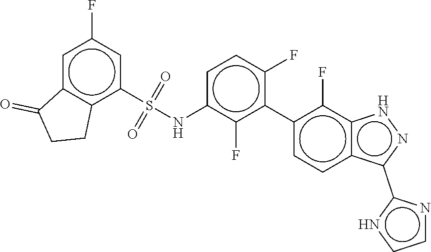 | 541.48 |
| 4N | 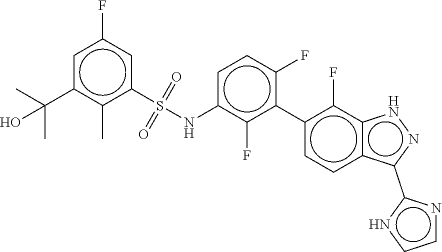 | 559.54 |
| 4O | 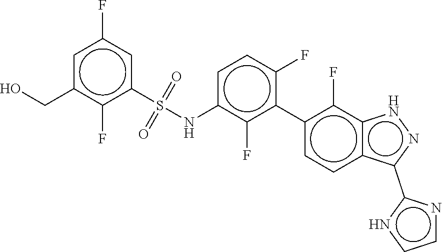 | 535.45 |
| 4P | 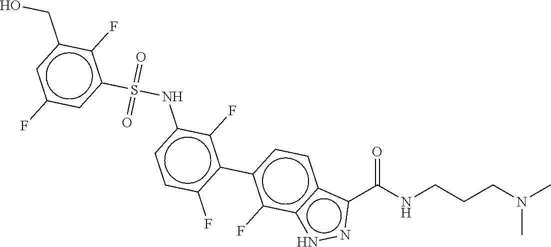 | 597.56 |
| 4Q | 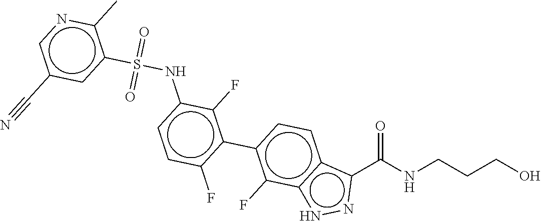 | 544.51 |
| 4R | 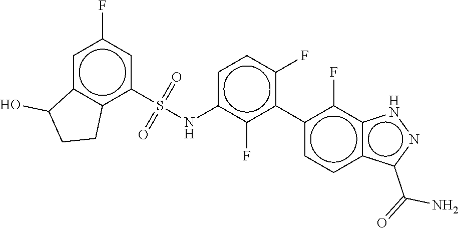 | 520.46 |
| 4S | 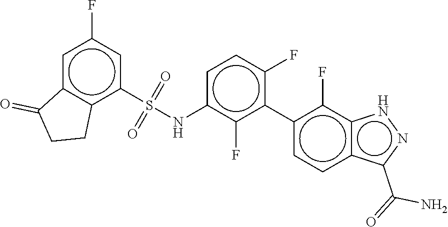 | 518.44 |
| 4T | 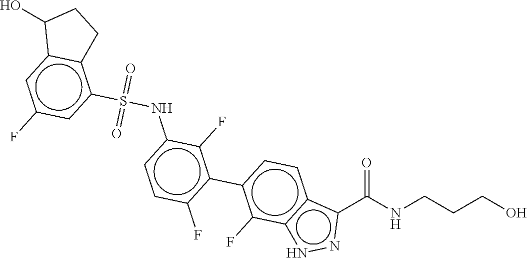 | 578.54 |
| 4U | 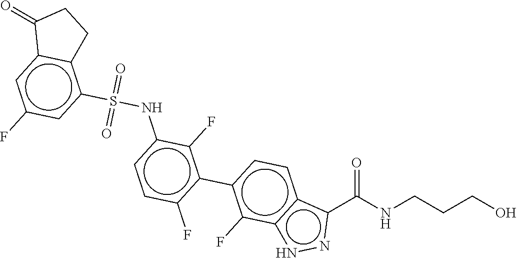 | 576.52 |
| 4V | 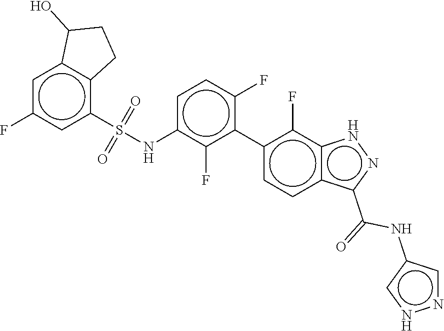 | 586.52 |
| 4W | 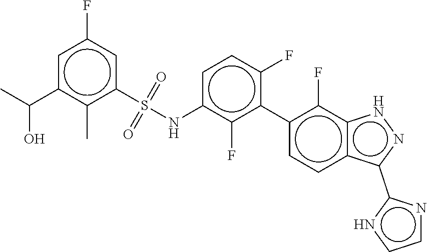 | 545.51 |
| 4X | 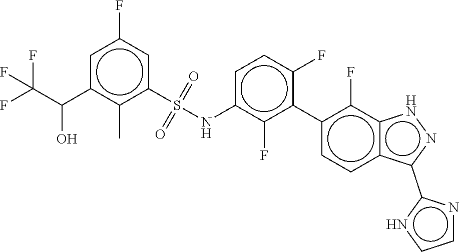 | 599.48 |
| 4Y | 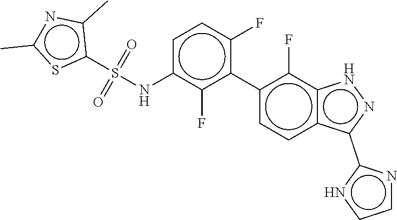 | 504.51 |
| 4Z | 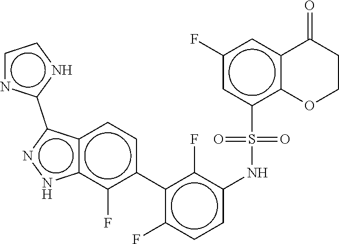 | 557.48 |
| 4AA | 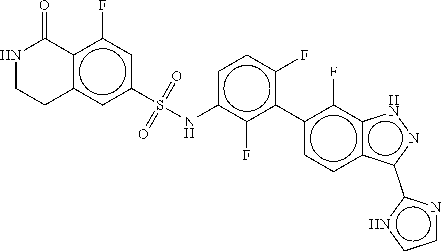 | 556.49 |
| 4AB | 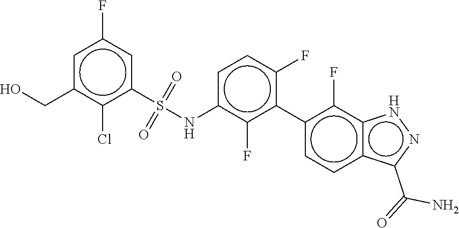 | 528.86 |
| 4AC | 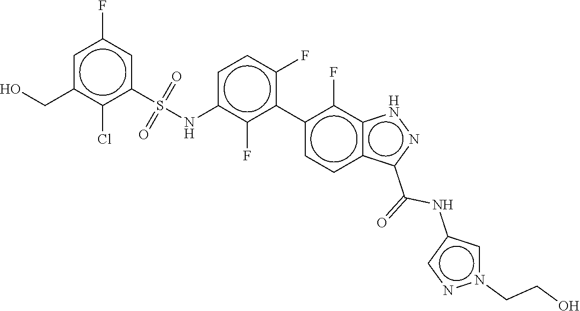 | 638.98 |
| 4AD | 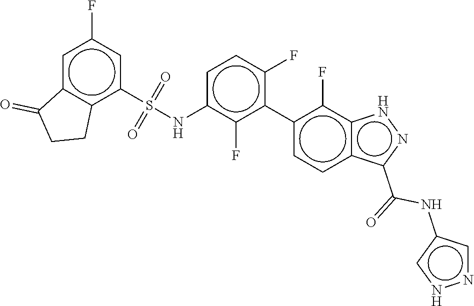 | 584.5 |
| 4AE | 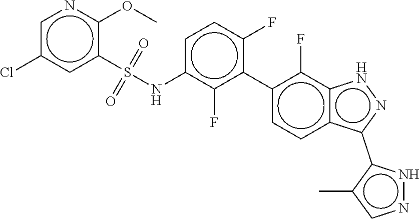 | 548.92 |
| 4AF | 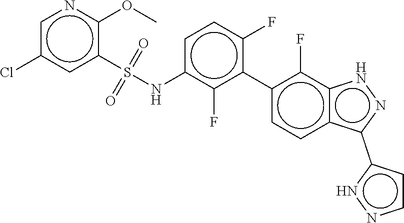 | 534.9 |
| 4AG | 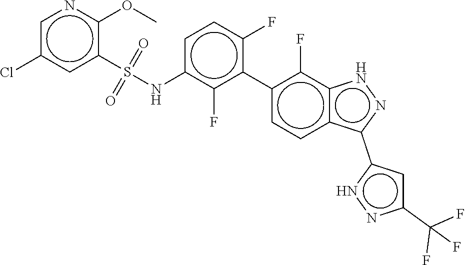 | 602.9 |
| 4AH | 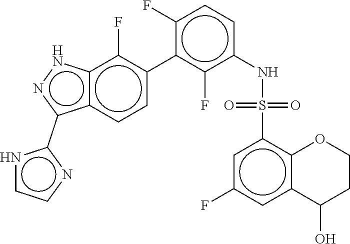 | 559.49 |
| 4AI | 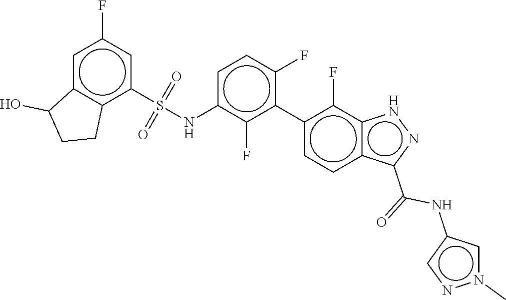 | 600.54 |
| 4AJ | 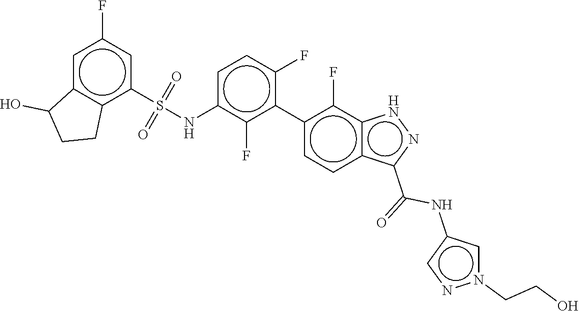 | 630.57 |
| 4AK | 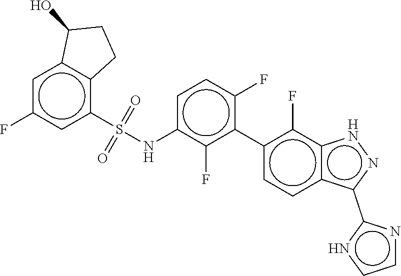 | 543.49 |
| 4AL | 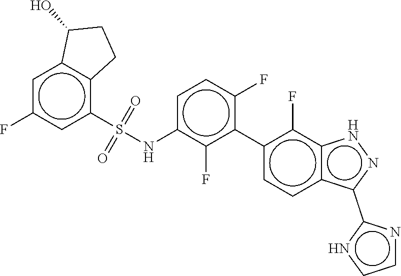 | 543.49 |
| 4AM | 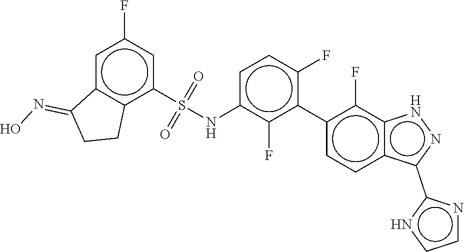 | 556.49 |
| 4AN | 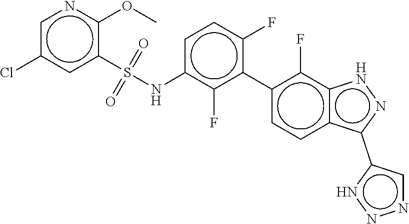 | 535.89 |
| 4AO | 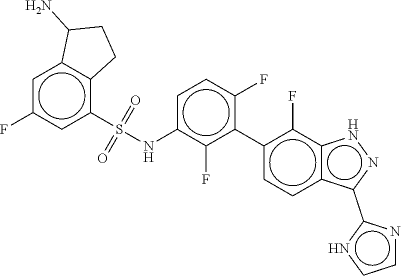 | 542.51 |
| 4AP | 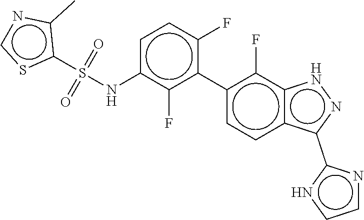 | 490.48 |
| 4AQ | 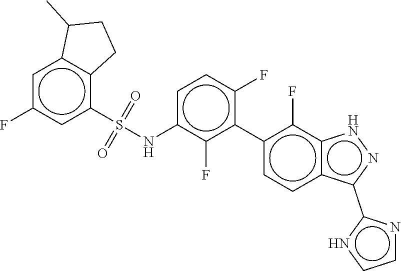 | 541.52 |
| 4AR | 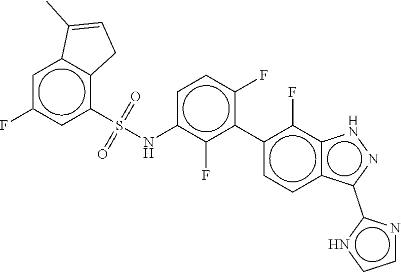 | 539.5 |
| 4AS | 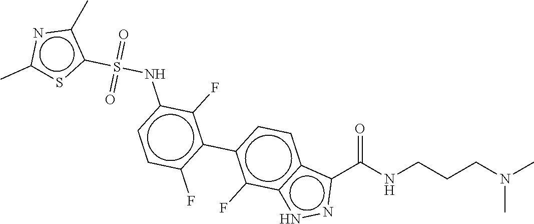 | 566.62 |
| 4AT | 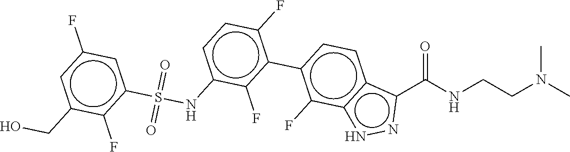 | 583.53 |
| 4AU | 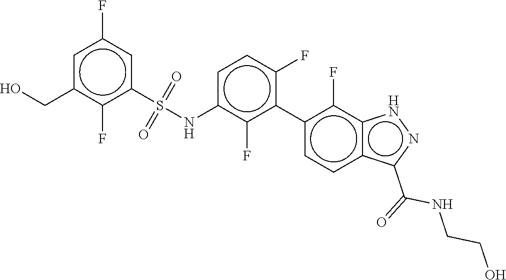 | 556.46 |
| 4AV | 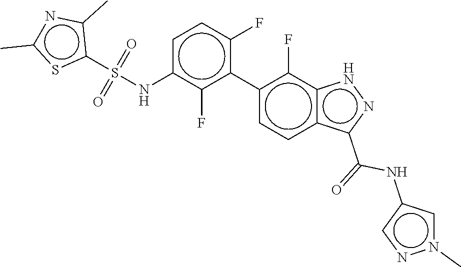 | 561.56 |
| 4AW | 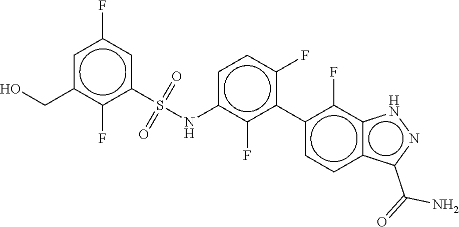 | 512.41 |
| 4AX | 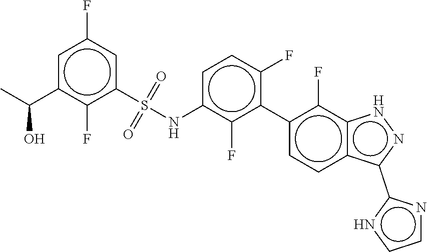 | 549.47 |
| 4AY | 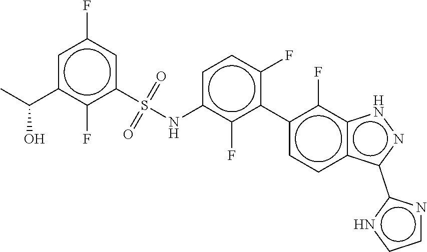 | 549.47 |
| 4AZ | 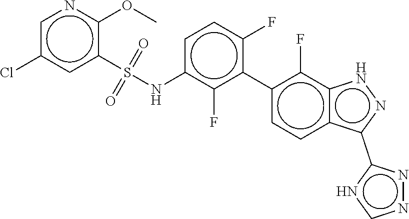 | 535.89 |
| 4BA | 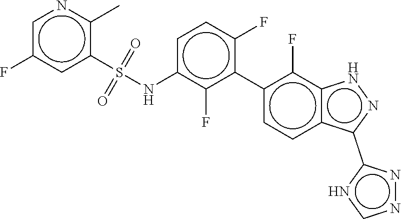 | 503.43 |
| 4BB | 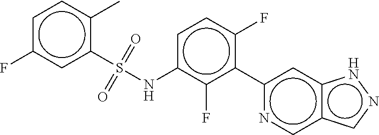 | 418.39 |

### Example 40—Synthesis of 6-(3-benzenesulfonamido-2,6-difluorophenyl)-7-fluoro-N-methyl-1H-indazole-3-carboxamide

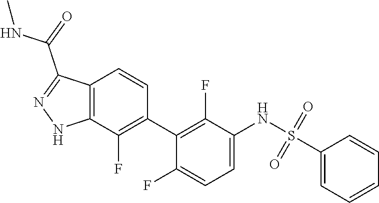 [SMILES: CNC(=O)c1n[nH]c2c(F)c(-c3c(F)ccc(NS(=O)(=O)c4ccccc4)c3F)ccc12]

### Part I—Synthesis of 6-(3-benzenesulfonamido-2,6-difluorophenyl)-7-fluoro-N-methyl-1-[[2-(trimethylsilyl)ethoxy]methyl]indazole-3-carboxamide

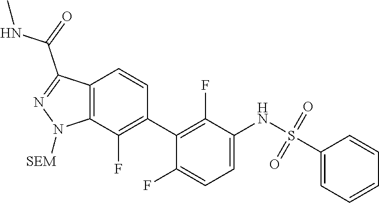 [SMILES: CNC(=O)c1nn(COCC[Si](C)(C)C)c2c(F)c(-c3c(F)ccc(NS(=O)(=O)c4ccccc4)c3F)ccc12]

Into a 10 mL vial, was placed 6-(3-amino-2,6-difluorophenyl)-7-fluoro-N-methyl-1-[[2-(trimethylsilyl)ethoxy]methyl] indazole-3-carboxamide (See Example 43). (310 mg, 0.7 mmol, 1 equiv), DCM (6 mL), pyridine (544 mg, 6.9 mmol, 10 equiv), benzenesulfonyl chloride (182 mg, 1 mmol, 1.5 equiv). The resulting solution was stirred for 30 min at room temperature, then was diluted with 10 mL of H₂O. The resulting solution was extracted with 3×10 mL of dichloromethane, and the extracts dried over anhydrous sodium sulfate. The resulting mixture was concentrated to yield 6-(3-benzenesulfonamido-2,6-difluorophenyl)-7-fluoro-N-methyl-1-[[2-(trimethylsilyl)ethoxy]methyl]indazole-3-carboxamide as a solid (325 mg, 80%). LCMS-PH-HBC-007-1 (ES, m/z):591 [M+H]+.

### Part II—Synthesis of 6-(3-benzenesulfonamido-2,6-difluorophenyl)-7-fluoro-N-methyl-1H-indazole-3-carboxamide

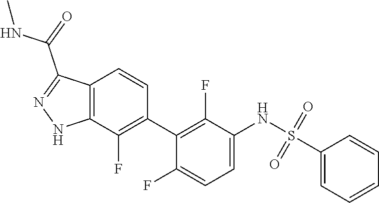 [SMILES: CNC(=O)c1n[nH]c2c(F)c(-c3c(F)ccc(NS(=O)(=O)c4ccccc4)c3F)ccc12]

Into a 50-mL round-bottom flask, was placed 6-(3-benzenesulfonamido-2,6-difluorophenyl)-7-fluoro-N-methyl-1-[[2-(trimethylsilyl)ethoxy]methyl]indazole-3-carboxamide (325 mg, 0.55 mmol, 1 equiv), CH₂Cl₂ (10 mL) and TFA (10 mL). The solution was stirred overnight at room temperature. The resulting solution was diluted with 15 mL of H₂O. The pH of the solution was adjusted to 8 with saturated aqueous NaHCO₃ and was extracted with 3×15 mL of dichloromethane. The extracts were dried over anhydrous sodium sulfate and concentrated. The residue was dissolved in 4 mL of CH₃OH. The crude product was purified by Prep-HPLC with the following conditions Column, Sunfire C18, 30*100 mm, 5 um; mobile phase, water (0.1% FA) and CH₃CN; Gradient: 10% B to 40% in 8 min; Flow rate: 25 mL/min; Detector, 220 nm. This resulted in 6-(3-benzenesulfonamido-2,6-difluorophenyl)-7-fluoro-N-methyl-1H-indazole-3-carboxamide as a white solid (38.5 mg, 15%). LCMS-PH-HBC-007-0 (ES, m/z):461[M+H]+. ¹H NMR-PH-HBC-007-0 (300 MHz, DMSO-d₆): δ 14.30 (s, 1H), 10.28 (s, 1H), 8.51 (d, J=4.9 Hz, 1H), δ 8.03 (d, J=8.4 Hz, 1H), 7.79-7.50 (m, 5H), 7.39 (td, J=8.9, 5.9 Hz, 1H), 7.29-7.18 (m, 1H), 7.13-6.98 (m, 1H), 2.83 (d, J=4.7 Hz, 3H).

### Example 41—Synthesis of 6-[2,6-Difluoro-3-[3-(hydroxymethyl)benzenesulfonamido]phenyl]-7-fluoro-N-methyl-1H-indazole-3-carboxamide

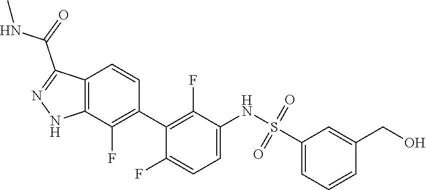 [SMILES: CNC(=O)c1n[nH]c2c(F)c(-c3c(F)ccc(NS(=O)(=O)c4cccc(CO)c4)c3F)ccc12]

### Part I—Synthesis of 3-(hydroxymethyl)benzenesulfonyl chloride

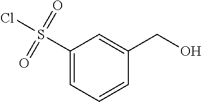 [SMILES: O=S(=O)(Cl)c1cccc(CO)c1]

Into a 50-mL round-bottom flask, 3-(chlorosulfonyl)benzoic acid (0.3 g, 1.4 mmol, 1 equiv) was placed THF (6 mL). This was followed by the addition of BH₃-THF (1.4 mL, 1.4 mmol, 1 equiv, 1M in THF) at 0 degrees C. in 10 min. The resulting solution was stirred for overnight at room temperature. The reaction was then quenched by the addition of water/ice and diluted with of 2M HCl (5 ml). The resulting solution was extracted with 3×10 mL of ethyl acetate, and the extracts were dried over anhydrous sodium sulfate. The resulting mixture was evaporated to give 3-(hydroxymethyl)benzenesulfonyl chloride (80 mg, 28%) as an off white solid.

### Part II—Synthesis of 6-[2,6-difluoro-3-[3-(hydroxymethyl)benzenesulfonamido]phenyl]-7-fluoro-N-methyl-1-[[2-(trimethylsilyl)ethoxy]methyl] indazole-3-carboxamide

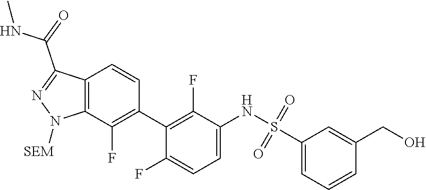 [SMILES: CNC(=O)c1nn(COCC[Si](C)(C)C)c2c(F)c(-c3c(F)ccc(NS(=O)(=O)c4cccc(CO)c4)c3F)ccc12]

Into a 50-mL round-bottom flask, was placed 6-(3-amino-2,6-difluorophenyl)-7-fluoro-N-methyl-1-[[2-(trimethylsilyl)ethoxy]methyl]indazole-3-carboxamide (See Example 43). (300 mg, 0.7 mmol, 1 equiv), CH₂Cl₂ (6 mL), pyridine (527 mg, 6.7 mmol, 10 equiv), 3-(hydroxymethyl)benzenesulfonyl chloride (206 mg, 1 mmol, 1.5 equiv). The resulting solution was stirred for 30 min at room temperature. The resulting solution was diluted with 10 mL of H₂O and extracted with 3×10 mL of dichloromethane. The extracts were dried over anhydrous sodium sulfate and concentrated to give 6-[2,6-difluoro-3-[3-(hydroxymethyl)benzenesulfonamido]phenyl]-7-fluoro-N-methyl-1-[[2-(trimethylsilyl)ethoxy]methyl] indazole-3-carboxamide (351 mg, 85%) as a yellow solid. LCMS (ES, m/z): 621 [M+H]+.

### Part III—Synthesis of 6-[2,6-Difluoro-3-[3-(hydroxymethyl)benzenesulfonamido]phenyl]-7-fluoro-N-methyl-1H-indazole-3-carboxamide

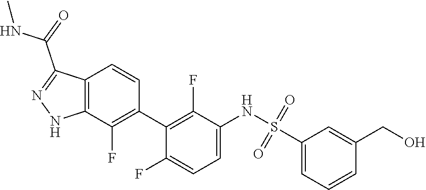 [SMILES: CNC(=O)c1n[nH]c2c(F)c(-c3c(F)ccc(NS(=O)(=O)c4cccc(CO)c4)c3F)ccc12]

Into a 50-mL round-bottom flask, was placed 6-[2,6-difluoro-3-[3-(hydroxymethyl)benzenesulfonamido]phenyl]-7-fluoro-N-methyl-1-[[2-(trimethylsilyl)ethoxy]methyl]indazole-3-carboxamide (350 mg), CH₂Cl₂ (10 mL) and TFA (10 mL). The resulting solution was stirred overnight at room temperature. The resulting solution was diluted with 15 mL of H₂O and the pH adjusted to 8 with saturated aqueous NaHCO₃. The resulting solution was extracted with 3×15 mL of dichloromethane, and the extracts dried over anhydrous sodium sulfate. After concentration, the residue was dissolved in 4 mL of CH₃OH and purified by Prep-HPLC with the following conditions: Column, Sunfire C18, 30*100 mm, 5 um; mobile phase, water (0.1% NH₃.H₂O) and CH₃CN; Gradient: 5% B to 35% in 10 min; Flow rate: 25 mL/min; Detector, 220. 6-[2,6-Difluoro-3-[3-(hydroxymethyl)benzenesulfonamido]phenyl]-7-fluoro-N-methyl-1H-indazole-3-carboxamide (74 mg) was isolated as a white solid. LCMS (ES, m/z):491 [M+H]+. ¹H NMR-PH-HBC-008-0 (300 MHz, DMSO-d₆): δ 14.30 (s, 1H), 10.27 (s, 1H), 8.51 (d, J=4.9 Hz, 1H), 8.05 (s, 1H), 7.74 (s, 1H), 7.66-7.48 (m, 3H), 7.38 (d, J=5.9 Hz, 1H), 7.27 (d, J=9.0 Hz, 1H), 7.15-7.01 (m, 1H), 4.56 (d, J=5.6 Hz, 2H), 2.84 (d, J=4.7 Hz, 3H).

### Example 42—Synthesis of 6-[2,6-difluoro-3-[3-fluoro-5-(hydroxymethyl)benzenesulfonamido]phenyl]-7-fluoro-N-methyl-1H-indazole-3-carboxamide

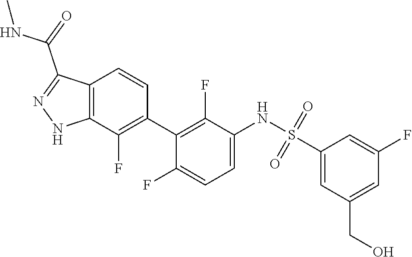 [SMILES: CNC(=O)c1n[nH]c2c(F)c(-c3c(F)ccc(NS(=O)(=O)c4cc(F)cc(CO)c4)c3F)ccc12]

### Part I—Synthesis of methyl 3-(benzylsulfanyl)-5-fluorobenzoate

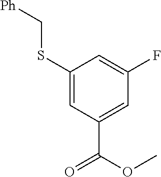 [SMILES: COC(=O)c1cc(F)cc(SCc2ccccc2)c1]

Into a 100-mL round-bottom flask purged and maintained with an inert atmosphere of nitrogen, was placed methyl 3-bromo-5-fluorobenzoate (1.50 g, 6.4 mmol, 1 equiv), Pd₂(dba)₃.CHCl₃ (0.15 g, 0.16 mmol, 0.025 equiv), Xantphos (0.19 g, 0.32 mmol, 0.05 equiv), dioxane (32 mL), DIEA (1.66 g, 12.9 mmol, 2 equiv), benzyl mercaptan (0.80 g, 6.4 mmol, 1 equiv). The resulting solution was stirred for 1 hr at 90 degrees C. The reaction mixture was cooled and concentrated. The residue was applied to a silica gel column with ethyl acetate/petroleum ether (1:8). The collected fractions were combined and concentrated to give methyl 3-(benzylsulfanyl)-5-fluorobenzoate (2.0 g, 96%) as light yellow oil. LCMS (ES, m/z):277 [M+H]+.

### Part II—Synthesis of [3-(benzylsulfanyl)-5-fluorophenyl]methanol

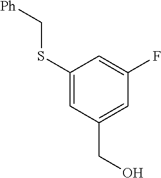 [SMILES: OCc1cc(F)cc(SCc2ccccc2)c1]

Into a 50-mL round-bottom flask, was placed methyl 3-(benzylsulfanyl)-5-fluorobenzoate (2.0 g, 7.2 mmol, 1 equiv) and THF (20 mL). This was followed by the addition of LAH (0.41 g, 10.8 mmol, 1.5 equiv) at 0 degrees C. The resulting solution was stirred for 30 min at 0 degrees C. The reaction was then quenched by the addition of water/ice and the pH was adjusted to 10 with NaOH (1M). The resulting solution was extracted with 3×20 mL of ethyl acetate, and the extracts were dried over anhydrous sodium sulfate. After concentration, the residue was applied to a silica gel column and eluted with ethyl acetate/petroleum ether (1:8). The appropriate fractions were combined and concentrated to give [3-(benzylsulfanyl)-5-fluorophenyl]methanol (1.6 g, 89%) as light yellow oil. LCMS (ES, m/z):249 [M+H]+.

### Part III—Synthesis of [3-(benzylsulfanyl)-5-fluorophenyl]methylacetate

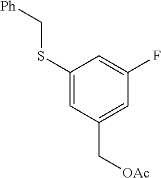 [SMILES: CC(=O)OCc1cc(F)cc(SCc2ccccc2)c1]

Into a 50-mL round-bottom flask, was placed [3-(benzylsulfanyl)-5-fluorophenyl]methanol (1.6 g, 6.4 mmol, 1 equiv), CH₂Cl₂ (32 mL), DMAP (0.08 g, 0.6 mmol, 0.1 equiv) and acetic anhydride (0.86 g, 8.4 mmol, 1.3 equiv). The resulting solution was stirred for 40 min at 25 degrees C. The mixture was concentrated and the residue applied to a silica gel column, eluting with ethyl acetate/petroleum ether (1:2). The collected fractions were combined and concentrated to give [3-(benzylsulfanyl)-5-fluorophenyl]methylacetate (1.6 g (86%) as light yellow oil. LCMS (ES, m/z):291 [M+H]+.

### Part IV—Synthesis of [3-(chlorosulfonyl)-5-fluorophenyl]methyl acetate

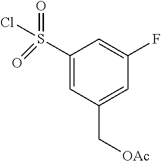 [SMILES: CC(=O)OCc1cc(F)cc(S(=O)(=O)Cl)c1]

Into a 50-mL round-bottom flask, was placed [3-(benzylsulfanyl)-5-fluorophenyl]methyl acetate (1.60 g, 5.5 mmol, 1 equiv), acetic acid (12 mL), H₂O (6 mL) and NCS (2.94 g, 22 mmol, 4 equiv). The resulting solution was stirred for 30 min at 25 degrees C., then was diluted with 10 mL of H₂O. The mixture was extracted with 3×15 mL of ethyl acetate, and the extracts washed with 1×20 ml of saturated aqueous NaHCO₃. After drying over anhydrous sodium sulfate, the solution was concentrated to give [3-(chlorosulfonyl)-5-fluorophenyl]methyl acetate (800 mg, 54%) as a light yellow oil.

### Part V—Synthesis of [3-([2,4-difluoro-3-[7-fluoro-3-(methylcarbamoyl)-1-[[2-(trimethylsilyl)ethoxy]methyl]indazol-6-yl]phenyl]sulfamoyl)-5-fluorophenyl] methyl acetate

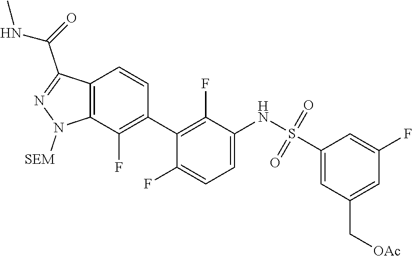

Into a 50-mL round-bottom flask, was placed 6-(3-amino-2,6-difluorophenyl)-7-fluoro-N-methyl-1-[[2-(trimethylsilyl)ethoxy]methyl]indazole-3-carboxamide (See Example 43). (0.80 g, 1.8 mmol, 1 equiv), CH₂Cl₂ (18 mL), pyridine (1.4 g, 18 mmol, 10 equiv), [3-(chlorosulfonyl)-5-fluorophenyl]methyl acetate (0.71 g, 2.7 mmol, 1.5 equiv). The resulting solution was stirred for 30 min at 25 degrees C. then diluted with 15 mL of H₂O. The mixture was extracted with 3×15 mL of dichloromethane, and the extracts dried over anhydrous sodium sulfate. Concentration gave [3-([2,4-difluoro-3-[7-fluoro-3-(methylcarbamoyl)-1-[[2-(trimethylsilyl)ethoxy]methyl]indazol-6-yl]phenyl]sulfamoyl)-5-fluorophenyl] methyl acetate (0.78 g, 65%) as a light yellow solid. LCMS (ES, m/z):681 [M+H]+.

### Part VI—6-(2,6-difluoro-3-((3-fluoro-5-(hydroxymethyl)phenyl) sulfonamido)phenyl)-7-fluoro-N-methyl-1-((2-(trimethylsilyl)ethoxy)methyl)-1H-indazole-3-carboxamide

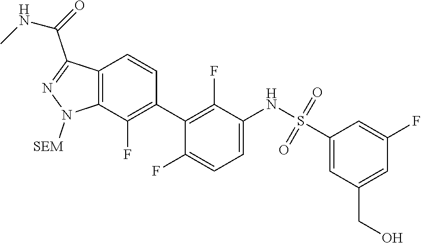

Into a 50-mL round-bottom flask, was placed [3-([2,4-difluoro-3-[7-methyl-3-(methylcarbamoyl)-1-[[2-(trimethylsilyl)ethoxy]methyl]indazol-6-yl]phenyl]sulfamoyl)-5-fluorophenyl]methyl acetate (0.78 g, 1.2 mmol, 1 equiv), LiOH (0.06 g, 2.5 mmol, 2.2 equiv), CH₃OH (8 mL), THE (8 mL) and H₂O (8 mL). The solution was stirred for 1.5 hr at 25 degrees C. and then concentrated. The resulting solution was diluted with 5 mL of H₂O, and the pH adjusted to 6 with HCl (1M). The solution was extracted with 3×15 mL of ethyl acetate, and the extracts were dried over anhydrous sodium sulfate. Concentration gave 6-(2,6-difluoro-3-((3-fluoro-5-(hydroxymethyl)phenyl) sulfonamido)phenyl)-7-fluoro-N-methyl-1-((2-(trimethylsilyl)ethoxy)methyl)-1H-indazole-3-carboxamide (0.53 g, 68%) as a brown solid. LCMS (ES, m/z):639 [M+H]+.

### Part VII—Synthesis of 6-[2,6-difluoro-3-[3-fluoro-5-(hydroxymethyl)benzenesulfonamido]phenyl]-7-fluoro-N-methyl-1H-indazole-3-carboxamide

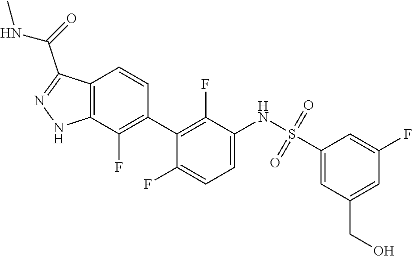

Into a 50-mL round-bottom flask, was placed 6-[2,6-difluoro-3-[3-fluoro-5-(hydroxymethyl)benzenesulfonamido] phenyl]-7-fluoro-N-methyl-1-[[2-(trimethylsilyl)ethoxy]methyl]indazole-3-carboxamide (150 mg, 0.3 mmol, 1 equiv), CH₂Cl₂ (2 mL) and TFA (2 mL). The resulting solution was stirred for 40 min at 25 degrees C., then quenched by the addition of 10 mL of water/ice. The pH was adjusted to 8 with NaHCO₃ and the mixture extracted with 3×10 mL of dichloromethane. The extracts were dried over anhydrous sodium sulfate and concentrated. The residue was dissolved in 5 mL of CH₃OH and purified by Prep-HPLC with the following conditions: Column, Sunfire C18, 30*100 mm, 5 um; mobile phase, water (0.1% NH₄HCO₃) and CH₃CN; Gradient: 20% B to 50% in 10 min; Flow rate: 25 mL/min; Detector, 220 nm. 6-[2,6-difluoro-3-[3-fluoro-5-(hydroxymethyl)benzenesulfonamido]phenyl]-7-fluoro-N-methyl-1H-indazole-3-carboxamide (69 mg) was isolated as a white solid. LCMS (ES, m/z):509 [M+H]+. ¹H NMR-PH-HBC-009-0 (300 MHz, DMSO-d₆): δ 14.30 (s, 1H), 10.43 (s, 1H), 8.51 (d, J=4.9 Hz, 1H), 8.00 (s, 1H), 7.50 (s, 1H), 7.43 (d, J=44.6 Hz, 3H), 7.21 (s, 1H), 7.06 (s, 1H), 4.48 (s, 2H), 2.80 (t, J=15.9 Hz, 3H).

### Example 43—Synthesis of 6-(3-amino-2-fluorophenyl)-7-fluoro-N-methyl-1-[[2-(trimethylsilyl)ethoxy]methyl]indazole-3-carboxamide

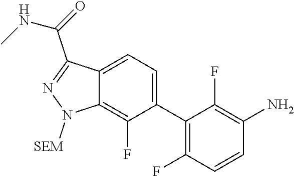

### Part I—Synthesis of 4-bromo-2,3-difluorobenzaldehyde

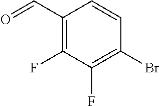 [SMILES: O=Cc1ccc(Br)c(F)c1F]

To a stirred solution of 1-bromo-2,3-difluorobenzene (100 g, 518 mmol, 1 equiv) in THE (1.2 L) was added LDA (285 mL, 570 mmol, 1.1 equiv, 2 M in hexane) dropwise at −78 degrees C. over 1 h under N₂ atmosphere. The resulting solution was stirred for 1 h at −78 degrees C. To the above mixture was added DMF (57.0 g, 823 mmol, 1.5 equiv) dropwise over 1 h at −78 degrees C. The resulting mixture was stirred for additional 1 h at −70 degrees C. The reaction was quenched with aqueous saturated NH₄Cl (500 mL). The resulting mixture was extracted with EA (3×500 mL). The combined organic layers were washed with brine (1×500 mL), dried over anhydrous Na₂SO₄. After filtration, the filtrate was concentrated under reduced pressure. The residue was purified by silica gel column chromatography, eluted with PE:EA (100:0-95:5) to afford 4-bromo-2,3-difluorobenzaldehyde (73 g, 64%) as a yellow solid.

### Part II—Synthesis of (E)-[(4-bromo-2,3-difluorophenyl)methylidene](methoxy)amine

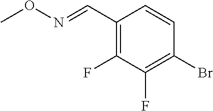

A mixture of 4-bromo-2,3-difluorobenzaldehyde (73 g, 330 mmol, 1 equiv), O-methylhydroxylamine hydrochloride (30.4 g, 363 mmol, 1.1 equiv) and K₂CO₃ (54.8 g, 396 mmol, 1.2 equiv) in DME (700 mL) was stirred for 2 h at 40 degrees C. The resulting mixture was filtered, and the filter cake washed with EA (2×200 mL). The combined filtrates were concentrated under reduced pressure to give (E)-[(4-bromo-2,3-difluorophenyl)methylidene](methoxy)amine (78.0 g, crude) as a red semi-solid.

### Part III—Synthesis of 6-bromo-7-fluoro-1H-indazole

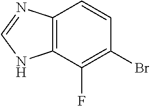 [SMILES: Fc1c(Br)ccc2nc[nH]c12]

A mixture of (E)-[(4-bromo-2,3-difluorophenyl)methylidene](methoxy)amine (73 g, 292 mmol, 1 equiv) and hydrazine hydrate (300 mL, 85%) in DME (750 mL) was stirred for overnight at 90 degrees C. The reaction was concentrated under reduced pressure. The residue was diluted with water (500 mL). The resulting mixture was extracted with EA (3×500 mL). The combined organic layers were washed with brine (2×200 mL), dried over anhydrous Na₂SO₄. After filtration, the filtrate was concentrated under reduced pressure. The resulting solid was washed with ether (1×200 mL), dried under vacuum to give 6-bromo-7-fluoro-1H-indazole (35 g, 56%) as a light yellow solid. LCMS (ES, m/z): [M+H]⁺: 215.

### Part IV—Synthesis of 6-bromo-7-fluoro-3-iodo-1H-indazole

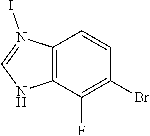

To a stirred solution of 6-bromo-7-fluoro-1H-indazole (35 g, 163 mmol, 1 equiv) and KOH (20.1 g, 358 mmol, 2.2 equiv) in DMF (600 mL) was added I₂ (82.3 g, 324 mmol, 2 equiv) in portions below 5 degrees C. The reaction was stirred for overnight at room temperature. The reaction was quenched with aqueous saturated Na₂SO₃ (1000 mL). The resulting mixture was extracted with EA (3×500 mL). The combined organic layers were washed with brine (1×200 mL), dried over anhydrous Na₂SO₄. After filtration, the filtrate was concentrated under reduced pressure. The residue was purified by silica gel column chromatography, eluted with PE:EA (9:1) to afford 6-bromo-7-fluoro-3-iodo-1H-indazole (53 g, 96%) as a yellow solid. LCMS (ES, m/z): [M+H]⁺: 341.

### Part V—Synthesis of 6-bromo-7-fluoro-3-iodo-1-[[2-(trimethylsilyl)ethoxy]methyl]indazole

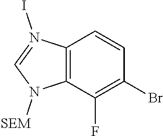

To a stirred solution of 6-bromo-7-fluoro-3-iodo-1H-indazole (530 g, 155 mmol, 1 equiv) in THE (500 mL) was added NaH (8.1 g, 202 mmol, 1.3 equiv, 60%) in portions at 0 degrees C. The resulting mixture was stirred for 0.5 h at 0 degrees C. To the above mixture was added SEMCl (38.9 g, 233 mmol, 1.5 equiv) dropwise over 30 min at 0 degrees C. The resulting mixture was stirred for additional 2 h at room temperature. The reaction was quenched with aqueous saturated NH₄Cl (300 mL). The resulting mixture was extracted with EA (3×200 mL). The combined organic layers were washed with brine (1×100 mL), dried over anhydrous Na₂SO₄. After filtration, the filtrate was concentrated under reduced pressure. The residue was purified by silica gel column chromatography, eluted with PE:EA (100:0-50:1) to afford 6-bromo-7-fluoro-3-iodo-1-[[2-(trimethylsilyl)ethoxy]methyl]indazole (49.6 g, 68%) as a yellow oil. LCMS (ES, m/z): [M+H]⁺: 471.

### Part VI—Synthesis of methyl 6-bromo-7-fluoro-1-[[2-(trimethylsilyl)ethoxy]methyl]indazole-3-carboxylate

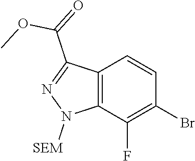 [SMILES: COC(=O)c1nn(COCC[Si](C)(C)C)c2c(F)c(Br)ccc12]

To a solution of 6-bromo-7-fluoro-3-iodo-1-[[2-(trimethylsilyl)ethoxy]methyl]indazole (49.6 g, 105 mmol, 1 equiv) in MeOH (700 mL) was added TEA (53 g, 526 mmol, 5 equiv), Pd(dppf)Cl₂ (7.7 g, 10.5 mmol, 0.1 equiv) in a pressure tank. The mixture was purged with nitrogen for 3 minutes and then was pressurized to 20 atm with carbon monoxide at 60 degrees C. for 4 h. The reaction mixture was cooled to room temperature and filtered to remove insoluble solids. The filtrate was concentrated under reduced pressure. The residue was purified by silica gel column chromatography, eluted with PE:EA (20:1) to afford methyl 6-bromo-7-fluoro-1-[[2-(trimethylsilyl)ethoxy]methyl]indazole-3-carboxylate (36.5 g, 86%) as a white solid. LCMS (ES, m/z): [M+H]⁺: 403.

### Part VII—Synthesis of methyl 7-fluoro-6-(4,4,5,5-tetramethyl-1,3,2-dioxaborolan-2-yl)-1-[[2-(trimethylsilyl)ethoxy]methyl]indazole-3-carboxylate

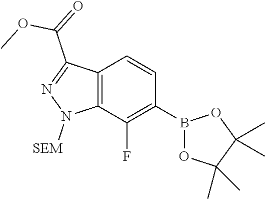

Into a 250-mL round-bottom flask purged and maintained with an inert atmosphere of nitrogen, was placed methyl methyl 6-bromo-7-fluoro-1-[[2-(trimethylsilyl)ethoxy]methyl]indazole-3-carboxylate (23 g, 57 mmol, 1 equiv), bis(pinacolato)diboron (29 g, 114 mmol, 2 equiv), Pd (dppf)Cl₂ (8.35 g, 11.4 mmol, 0.2 equiv), KOAc (8.40 g, 85.5 mmol, 1.5 equiv), dioxane (300 mL, 23.6 mmol, 190 equiv). The resulting solution was stirred for overnight at 100 degrees C. The reaction mixture was cooled. The solids were filtered out and washed with dioxane (10 mL×2). The resulting mixture was concentrated. The residue was dissolved in 200 mL of cyclohexane. The solids were filtered and washed with cyclohexane (10 mL×3). The resulting mixture was concentrated. This resulted in crude methyl 7-fluoro-6-(4,4,5,5-tetramethyl-1,3,2-dioxaborolan-2-yl)-1-[[2-(trimethylsilyl)ethoxy] methyl]indazole-3-carboxylate 15.2 g (59.18%) as a brown oil. LCMS (ES, m/z): [M+H]⁺: 451.

### Part VIII—Synthesis of methyl 6-(3-amino-2,6-difluorophenyl)-7-fluoro-1-[[2-(trimethylsilyl) ethoxy]methyl]indazole-3-carboxylate

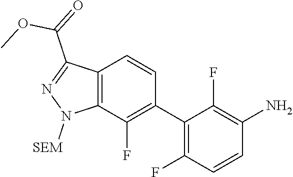 [SMILES: COC(=O)c1nn(COCC[Si](C)(C)C)c2c(F)c(-c3c(F)ccc(N)c3F)ccc12]

Into a 10-mL vial purged and maintained with an inert atmosphere of nitrogen, was placed methyl 7-fluoro-6-(4,4,5,5-tetramethyl-1,3,2-dioxaborolan-2-yl)-1-[[2-(trimethylsilyl)ethoxy]methyl]indazole-3-carboxylate (15.2 g, 33.7 mmol, 1 equiv), 3-bromo-2,4-difluoroaniline (5.6 g, 27 mmol, 0.8 equiv), SPhos Pd G3 (2.63 g, 3.4 mmol, 0.1 equiv), K₂CO₃ (7.0 g, 51 mmol, 1.5 equiv), SPhos (2.77 g, 6.8 mmol, 0.2 equiv), dioxane (100 mL) and H₂O (25 mL). The resulting solution was stirred for 1 hour at 100 degrees C. The reaction mixture was cooled and diluted with 50 mL of H₂O. The resulting solution was extracted with 3×150 mL of ethyl acetate, and the extracts were dried over anhydrous sodium sulfate. The resulting mixture was concentrated and the residue was applied onto a silica gel column, eluting with ethyl acetate/petroleum ether (1:2). Concentration of the appropriate fractions gave methyl 6-(3-amino-2,6-difluorophenyl)-7-fluoro-1-[[2-(trimethylsilyl)ethoxy]methyl]indazole-3-carboxylate as a brown oil (10.2 g, 67%). LCMS (ES, m/z): [M+H]⁺: 452.

### Part IX—Synthesis of 6-(3-amino-2-fluorophenyl)-7-fluoro-N-methyl-1-[[2-(trimethylsilyl)ethoxy] methyl]indazole-3-carboxamide

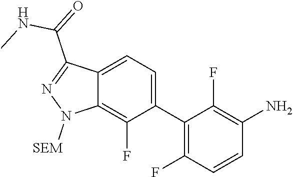 [SMILES: CNC(=O)c1nn(COCC[Si](C)(C)C)c2c(F)c(-c3c(F)ccc(N)c3F)ccc12]

Into a 10 mL vial, was placed methyl 6-(3-amino-2,6-difluorophenyl)-7-fluoro-1-((2-(trimethylsilyl)ethoxy)methyl)-1H-indazole-3-carboxylate (4.8 g, 10.6 mmol, 1 equiv), methylamine hydrate (10 mL). The resulting solution was stirred for overnight at room temperature. The resulting solution was extracted with 3×3 mL of ethyl acetate and the extracts were dried over anhydrous sodium sulfate. The filtrate was concentrated to give 6-(3-amino-2-fluorophenyl)-7-fluoro-N-methyl-1-[[2-(trimethylsilyl)ethoxy]methyl]indazole-3-carboxamide (4.1 g, 89%) as a brown oil. LCMS (ES, m/z): [M+H]+: 451.

### Example 44—Synthesis of N-(6-(3-(5-chloro-2-methoxypyridine-3-sulfonamido)-2,6-difluorophenyl)-7-fluoro-1H-indazol-3-yl)acetamide

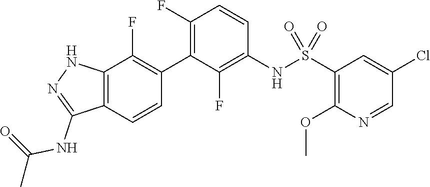 [SMILES: COc1ncc(Cl)cc1S(=O)(=O)Nc1ccc(F)c(-c2ccc3c(NC(C)=O)n[nH]c3c2F)c1F]

### Part I—Synthesis of N-(3-(3-amino-7-fluoro-1-((2-(trimethylsilyl)ethoxy)methyl)-1H-indazol-6-yl)-2,4-difluorophenyl)-5-chloro-2-methoxypyridine-3-sulfonamide

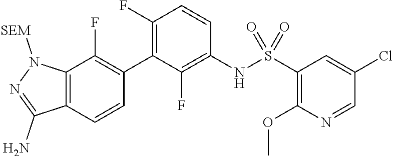 [SMILES: COc1ncc(Cl)cc1S(=O)(=O)Nc1ccc(F)c(-c2ccc3c(N)nn(COCC[Si](C)(C)C)c3c2F)c1F]

To a stirred solution of 6-(3-(5-chloro-2-methoxypyridine-3-sulfonamido)-2,6-difluorophenyl)-7-fluoro-1-((2-(trimethylsilyl)ethoxy)methyl)-1H-indazole-3-carboxylic acid (See Example 7) (200 mg, 0.3 mmol, 1 equiv) in DMF (5 mL) were added DPPA (128 mg, 0.47 mmol, 1.5 equiv), TEA (47 mg, 0.47 mmol, 1.5 equiv). The resulting solution was stirred for 1 hour at 100° C. The reaction was cooled to room temperature and poured into water (20 mL). The resulting mixture was extracted with ethyl acetate (3×20 mL). The combined organics were washed with brine (2×10 mL), dried over anhydrous Na₂SO₄. After filtration, the filtrate was concentration under reduced pressure. The residue was purified by column chromatography over silica gel (eluent: PE:EA=8:1) to afford N-(3-(3-amino-7-fluoro-1-((2-(trimethylsilyl)ethoxy)methyl)-1H-indazol-6-yl)-2,4-difluorophenyl)-5-chloro-2-methoxypyridine-3-sulfonamide (160 mg, 52%) as a white solid. LCMS (ES, m/z): [M+H]⁺: 614.

### Part II—Synthesis of N-(6-(3-(5-chloro-2-methoxypyridine-3-sulfonamido)-2,6-difluorophenyl)-7-fluoro-1-((2-(trimethylsilyl)ethoxy) methyl)-1H-indazol-3-yl)acetamide

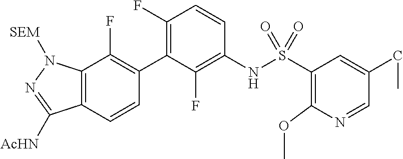 [SMILES: CCc1cnc(OC)c(S(=O)(=O)Nc2ccc(F)c(-c3ccc4c(NC(C)=O)nn(COCC[Si](C)(C)C)c4c3F)c2F)c1]

To a stirred solution of N-[3-(3-amino-7-fluoro-1-[[2-(trimethylsilyl) ethoxy]methyl]indazol-6-yl)-2,4-difluorophenyl]-5-chloro-2-methoxypyridine-3-sulfonamide (130 mg, 0.2 mmol, 1 equiv) and pyridine (33 mg, 0.4 mmol, 2 equiv) in THE (5 mL) was added acetyl chloride (20 mg, 0.25 mmol, 1.2 equiv) dropwise at 0° C. The resulting solution was stirred for 30 minutes at 0° C. The reaction was quenched with H₂O (1.0 mL) and concentrated under reduced pressure. The residue was purified by prep-TLC (PE:EA=1:1) to afford N-(6-(3-(5-chloro-2-methoxypyridine-3-sulfonamido)-2,6-difluorophenyl)-7-fluoro-1-((2-(trimethylsilyl)ethoxy) methyl)-1H-indazol-3-yl)acetamide (90 mg, 66%) as a white solid. LCMS (ES, m/z): [M+H]⁺: 656.

### Part III—Synthesis of N-(6-(3-(5-chloro-2-methoxypyridine-3-sulfonamido)-2,6-difluorophenyl)-7-fluoro-1H-indazol-3-yl)acetamide

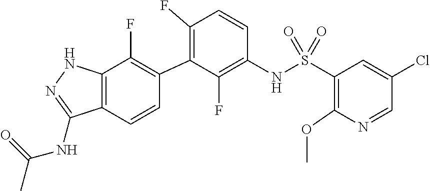 [SMILES: COc1ncc(Cl)cc1S(=O)(=O)Nc1ccc(F)c(-c2ccc3c(NC(C)=O)n[nH]c3c2F)c1F]

A mixture of N-(6-(3-(5-chloro-2-methoxypyridine-3-sulfonamido)-2,6-difluorophenyl)-7-fluoro-1-((2-(trimethylsilyl)ethoxy)methyl)-1H-indazol-3-yl)acetamide (90 mg, 0.14 mmol, 1 equiv) and TFA/DCM (1:4, 5 mL) was stirred for 0.5 hour at room temperature. The resulting mixture was concentrated. The residue was added to ammonia (7.0 M solution in MeOH) (5 mL) and stirred for 0.5 hour at room temperature. The resulting solution was concentrated. The residue was purified by prep-HPLC to afford N-(6-(3-(5-chloro-2-methoxypyridine-3-sulfonamido)-2,6-difluorophenyl)-7-fluoro-1H-indazol-3-yl)acetamide (70 mg, 59%) as a white solid. LCMS (ES, m/z): [M+H]⁺: 626. ¹H NMR (300 MHz, DMSO-d₆) δ 13.39 (s, 1H), 10.54 (s, 1H), 8.48 (d, J=2.6 Hz, 1H), 8.06 (d, J=2.6 Hz, 1H), 7.68 (d, J=8.4 Hz, 1H), 7.43 (td, J=8.9, 5.9 Hz, 1H), 7.31-7.15 (m, 1H), 6.91 (t, J=7.2 Hz, 1H), 3.90 (s, 3H), 2.13 (s, 3H).

### Example 45—Synthesis of 6-[3-[5-chloro-2-(difluoromethoxy)pyridine-3-sulfonamido]-2,6-difluorophenyl]-7-fluoro-N-methyl-1H-indazole-3-carboxamide

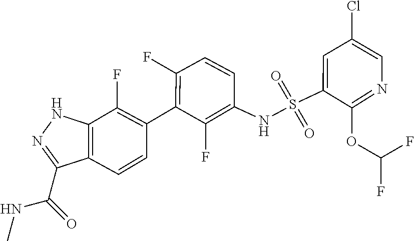 [SMILES: CNC(=O)c1n[nH]c2c(F)c(-c3c(F)ccc(NS(=O)(=O)c4cc(Cl)cnc4OC(F)F)c3F)ccc12]

### Part I—Synthesis of 3-Bromo-5-chloro-2-(difluoromethoxy)pyridine

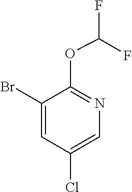 [SMILES: FC(F)Oc1ncc(Cl)cc1Br]

To a stirred solution of 3-bromo-5-chloropyridin-2-ol (10 g, 48 mmol, 1 equiv) and sodium 2-chloro-2,2-difluoroacetate (14.6 g, 96 mmol, 2 equiv) in DMF (100 mL) was added K₂CO₃ (9.95 g, 72 mmol, 1.5 equiv) in portions at room temperature. The resulting mixture was stirred for 2 h at 100 degrees C. The mixture was allowed to cool and was quenched with water (100 mL). The resulting mixture was extracted with EA (3×100 mL). The combined organic layers were washed with brine (2×100 mL), dried over anhydrous Na₂SO₄. After filtration, the filtrate was concentrated under reduced pressure. The residue was purified by prep-HPLC Flash-Prep-HPLC with the following conditions: Column, WelFlash TM C18-I, Spherical C18 20-40 m, 120 g; mobile phase, 0.1% FA and MeCN, from 20% increasing to 70% in 20 min; Detector, 220 nm. 3-Bromo-5-chloro-2-(difluoromethoxy)pyridine (3.3 g, 27%) was isolated as a brown oil.

### Part II—Synthesis of 3-(benzylsulfanyl)-5-chloro-2-(difluoromethoxy)pyridine

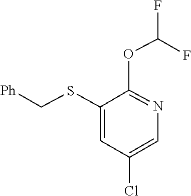 [SMILES: FC(F)Oc1ncc(Cl)cc1SCc1ccccc1]

To a stirred mixture of 3-bromo-5-chloro-2-(difluoromethoxy)pyridine (3.2 g, 12.4 mmol, 1 equiv) and benzyl mercaptan (1.54 g, 12.4 mmol, 1 equiv) in toluene (40 mL) were added DIEA (2.4 g, 18.6 mmol, 1.5 equiv), XantPhos (1.43 g, 2.5 mmol, 0.2 equiv) and Pd₂(dba)₃.CHCl₃ (1.3 g, 1.2 mmol, 0.1 equiv) at room temperature under N₂ atmosphere. The resulting mixture was stirred for 5 h at 115 degrees C. under N₂ atmosphere. The mixture was allowed to cool and was filtered. The filtrate was concentrated under reduced pressure. The residue was purified by silica gel column chromatography, eluting with PE:EA (20:1) to afford 3-(benzylsulfanyl)-5-chloro-2-(difluoromethoxy)pyridine (1.5 g, 40%) as a white solid. LCMS (ES, m/z): [M+H]⁺: 302.

### Part III—Synthesis of 5-chloro-2-(fluoromethoxy)pyridine-3-sulfonyl chloride

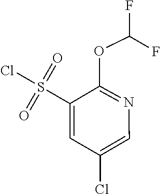 [SMILES: O=S(=O)(Cl)c1cc(Cl)cnc1OC(F)F]

To a stirred solution of 3-(benzylsulfanyl)-5-chloro-2-(difluoromethoxy)pyridine (500 mg, 1.7 mmol, 1 equiv) and H₂O (5 mL) in HOAc (15 mL) was added NCS (775 mg, 5.8 mmol, 3.5 equiv) in portions below 10 degrees C. The resulting solution was stirred for 4 h below 20 degrees C. The reaction was quenched with water (20 mL). The resulting solution was extracted with EA (3×50 mL). The combined organics were washed with brine (2×50 mL) and dried over anhydrous Na₂SO₄. After filtration, the filtrate was concentrated under reduced pressure to give 5-chloro-2-(fluoromethoxy)pyridine-3-sulfonyl chloride (345 mg, crude) as a yellow oil which was used in next step directly without further purification.

### Part IV—Synthesis of 6-[3-[5-chloro-2-(difluoromethoxy)pyridine-3-sulfonamido]-2,6-difluorophenyl]-7-fluoro-N-methyl-1-[[2-(trimethylsilyl)ethoxy]methyl]indazole-3-carboxamide

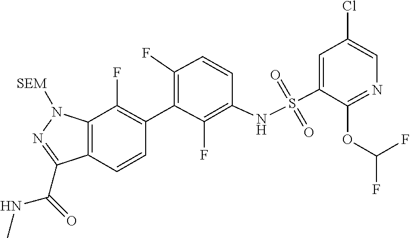 [SMILES: CNC(=O)c1nn(COCC[Si](C)(C)C)c2c(F)c(-c3c(F)ccc(NS(=O)(=O)c4cc(Cl)cnc4OC(F)F)c3F)ccc12]

To a stirred solution of 6-(3-amino-2,6-difluorophenyl)-7-fluoro-N-methyl-1-((2-(trimethylsilyl)ethoxy)methyl)-1H-indazole-3-carboxamide (366 mg, 0.8 mmol, 1 equiv) and pyridine (490 mg, 6.2 mmol, 7.7 equiv) in DCM (6 mL) was added a solution of 5-chloro-2-(difluoromethoxy)pyridine-3-sulfonyl chloride (340 mg, 1.2 mmol, 1.5 equiv) in DCM (2 mL) dropwise at 0 degrees C. The mixture was stirred for 2 h at room temperature. The mixture was concentrated under reduced pressure. The residue was purified by silica gel column chromatography, eluting with PE:EA (2:1) to give 6-[3-[5-chloro-2-(difluoromethoxy)pyridine-3-sulfonamido]-2,6-difluorophenyl]-7-fluoro-N-methyl-1-[[2-(trimethylsilyl)ethoxy]methyl]indazole-3-carboxamide (125 mg, 22%) as a yellow solid. LCMS (ES, m/z): [M+H]⁺: 692.

### Part V—Synthesis of 6-[3-[5-chloro-2-(difluoromethoxy)pyridine-3-sulfonamido]-2,6-difluorophenyl]-7-fluoro-N-methyl-1H-indazole-3-carboxamide

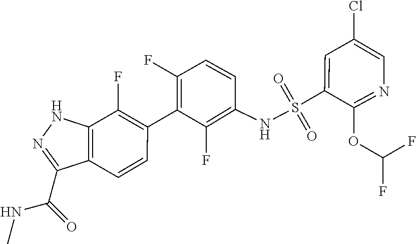 [SMILES: CNC(=O)c1n[nH]c2c(F)c(-c3c(F)ccc(NS(=O)(=O)c4cc(Cl)cnc4OC(F)F)c3F)ccc12]

To a stirred solution of 6-(3-((5-chloro-2-(difluoromethoxy)pyridine)-3-sulfonamido)-2,6-difluorophenyl)-7-fluoro-N-methyl-1-((2-(trimethylsilyl)ethoxy)methyl)-1H-indazole-3-carboxamide (121 mg, 0.18 mmol, 1 equiv) in DCM (4 mL) was added TFA (2 mL) dropwise at room temperature. The resulting solution was stirred overnight at room temperature. The mixture was concentrated under reduced pressure. The residue was purified by prep-HPLC with the following conditions Column, welch Vltimate XB-C18, 50×250 mm, 10 μm mobile phase, Mobile Phase A: 0.1% FA in Water, Mobile Phase B: CAN (35% up to 70% in 10 min) to afford 6-[3-[5-chloro-2-(difluoromethoxy)pyridine-3-sulfonamido]-2,6-difluorophenyl]-7-fluoro-N-methyl-1H-indazole-3-carboxamide (16.7 mg, 17%) as a white solid. LCMS (ES, m/z): [M−H]⁻: 560. ¹H NMR (300 MHz, DMSO-d₆) δ 14.30 (s, 1H), 10.37 (s, 1H), 8.52-8.48 (m, 2H), 8.07-7.65 (m, 3H), 7.46 (d, J=8.7 Hz, 1H), 7.23-7.13 (m, 2H), 2.84 (d, J=4.7 Hz, 3H).

### Example 46—Synthesis of 6-[3-[5-Cyano-2-(difluoromethoxy)pyridine-3-sulfonamido]-2,6-difluorophenyl]-7-fluoro-N-methyl-1H-indazole-3-carboxamide

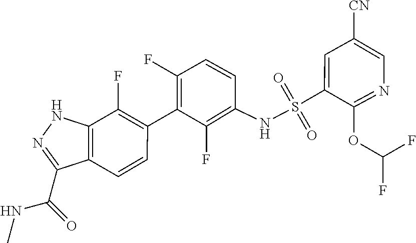 [SMILES: CNC(=O)c1n[nH]c2c(F)c(-c3c(F)ccc(NS(=O)(=O)c4cc(C#N)cnc4OC(F)F)c3F)ccc12]

### Part I—Synthesis of 6-hydroxypyridine-3-carbonitrile

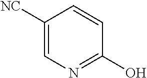

To a stirred mixture of 6-aminopyridine-3-carbonitrile (10 g, 84 mmol, 1 equiv) and conc. H₂SO₄ (12 mL, 225 mmol, 2.7 equiv) in H₂O (115 mL) was added NaNO₂ (9.9 g, 143 mmol, 1.7 equiv) in portions at 0-5 degrees C. under N₂ atmosphere. The mixture was stirred for 30 minutes at 100 degrees C. The mixture was allowed to cool down to room temperature and stirred for 1 h. The resulting solid was collected by filtration. The solid was washed with H₂O (2×100 mL) and dried under vacuum to give 6-hydroxypyridine-3-carbonitrile (7.2 g, 71%) as a white solid. ¹H NMR (300 MHz, DMSO-d₆) δ 12.42 (s, 1H), 8.25 (dd, J=2.6, 0.7 Hz, 1H), 7.66 (dd, J=9.6, 2.6 Hz, 1H), 6.42 (dd, J=9.6, 0.7 Hz, 1H).

### Part II—Synthesis 6-hydroxy-5-iodopyridine-3-carbonitrile

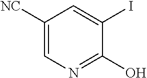

To a stirred mixture of 6-hydroxypyridine-3-carbonitrile (7.7 g, 64 mmol, 1 equiv) in DMF (100 mL) was added NIS (15.9 g, 71 mmol, 1.1 equiv) in portions at room temperature. The mixture was stirred for 4 h at 90 degrees C. The reaction was quenched with water (100 mL). The resulting mixture was extracted with EA (8×100 mL). The combined organic layers were washed with brine (2×50 mL) and dried over anhydrous Na₂SO₄. After filtration, the filtrate was concentrated under reduced pressure. The residue was purified by silica gel column chromatography, eluting with PE:EA (1:1˜0:1) to afford 6-hydroxy-5-iodopyridine-3-carbonitrile (12.5 g, 79% yield) as a light yellow solid. LCMS (ES, m/z): [M+H]⁺: 247.

### Part III—Synthesis of 6-(difluoromethoxy)-5-iodopyridine-3-carbonitrile

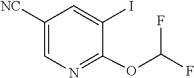 [SMILES: N#Cc1cnc(OC(F)F)c(I)c1]

To a stirred solution of 6-hydroxy-5-iodopyridine-3-carbonitrile (1.34 g, 5.5 mmol, 1 equiv) and difluoro(sulfo)acetic acid (2.9 g, 16 mmol, 3 equiv) in CH₃CN (15 mL) was added anhydrous Na₂SO₄ (1.55 g, 10.9 mmol, 2 equiv) in portions at room temperature under nitrogen atmosphere. The resulting mixture was stirred for 3 hours at 75 degrees C. The reaction was quenched with saturated aqueous NaHCO₃ (100 mL). The resulting mixture was extracted with EA (3×50 mL). The combined organic layers were washed with brine (2×20 mL) and dried over anhydrous Na₂SO₄. After filtration, the filtrate was concentrated under reduced pressure. The residue was purified by silica gel column chromatography, eluting with PE:EA to afford 6-(difluoromethoxy)-5-iodopyridine-3-carbonitrile (0.96 g, 59% yield) as a white solid.

### Part IV—Synthesis of 5-(benzylsulfanyl)-6-(difluoromethoxy)pyridine-3-carbonitrile

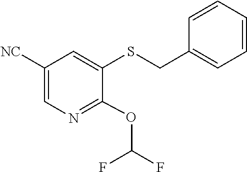 [SMILES: N#Cc1cnc(OC(F)F)c(SCc2ccccc2)c1]

To a stirred mixture of 6-(difluoromethoxy)-5-iodopyridine-3-carbonitrile (3.0 g, 10 mmol, 1 equiv), benzyl mercaptan (1.89 g, 15 mmol, 1.5 equiv) and DIEA (2.62 g, 20 mmol, 2 equiv) in toluene (50 mL) was added XantPhos (1.2 g, 2 mmol, 0.2 equiv) and Pd₂(dba)₃. CHCl₃ (1.05 g, 1 mmol, 0.1 equiv) under N₂ atmosphere. The resulting mixture was stirred for 4 h at 115 degrees C. The mixture was allowed to cool to room temperature and was quenched with water (200 mL). The resulting mixture was extracted with EA (3×100 mL). The combined organic layers were washed with brine (2×100 mL) and dried over anhydrous Na₂SO₄. After filtration, the filtrate was concentrated under reduced pressure. The crude product was purified by silica gel column chromatography, eluted with PE:EA=1:1 to give 5-(benzylsulfanyl)-6-(difluoromethoxy)pyridine-3-carbonitrile (2.70 g, 91%) as a yellow solid. LCMS (ES, m/z): [M+H]⁺: 293.

### Part V—Synthesis of 5-cyano-2-(difluoromethoxy)pyridine-3-sulfonyl chloride

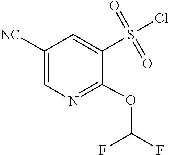 [SMILES: N#Cc1cnc(OC(F)F)c(S(=O)(=O)Cl)c1]

To a stirred mixture of 5-(benzylsulfanyl)-6-(difluoromethoxy)pyridine-3-carbonitrile (2.0 g, 6.8 mmol, 1 equiv) and H₂O (10 mL) in HOAc (50 mL) was added NCS (3.2 g, 24 mmol, 3.5 equiv) below 10 degrees C. The mixture was stirred for 4 h below 20 degrees C. The mixture was quenched with water (200 mL). The resulting mixture was extracted with EA (3×100 mL). The combined organic layers were washed with brine (2×100 mL) and dried over anhydrous Na₂SO₄. After filtration, the filtrate was concentrated under reduced pressure. The residue was purified by silica gel column chromatography, eluting with PE:EA (2:1) to afford 5-cyano-2-(difluoromethoxy)pyridine-3-sulfonyl chloride (0.85 g, 46%) as a white solid.

### Part VI—Synthesis of 6-[3-[5-cyano-2-(difluoromethoxy)pyridine-3-sulfonamido]-2,6-difluorophenyl]-7-fluoro-N-methyl-1-[[2-(trimethylsilyl)ethoxy]methyl]indazole-3-carboxamide

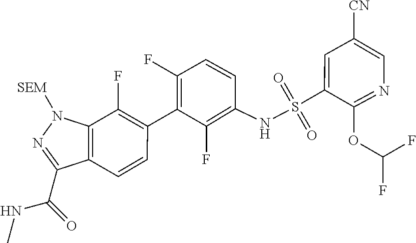 [SMILES: CNC(=O)c1nn(COCC[Si](C)(C)C)c2c(F)c(-c3c(F)ccc(NS(=O)(=O)c4cc(C#N)cnc4OC(F)F)c3F)ccc12]

To a stirred solution of 6-(3-amino-2,6-difluorophenyl)-7-fluoro-N-methyl-1-((2-(trimethylsilyl)ethoxy)methyl)-1H-indazole-3-carboxamide (960 mg, 2.1 mmol, 1 equiv) and pyridine (674 mg, 8.5 mmol, 4 equiv) in DCM (10 mL) was added a solution of 5-cyano-2-(difluoromethoxy)pyridine-3-sulfonyl chloride (859 mg, 3.2 mmol, 1.5 equiv) in DCM (5 mL) dropwise at 0 degrees C. The mixture was stirred for 2 h at room temperature. The reaction was quenched with water (50 mL). The resulting mixture was extracted with EA (3×20 mL). The combined organic layers were washed with brine (2×20 mL) and dried over anhydrous Na₂SO₄. After filtration, the filtrate was concentrated under reduced pressure. The residue was purified by silica gel column chromatography, eluting with PE:EA (2:1-1:1) to afford 6-[3-[5-cyano-2-(difluoromethoxy)pyridine-3-sulfonamido]-2,6-difluorophenyl]-7-fluoro-N-methyl-1-[[2-(trimethylsilyl)ethoxy]methyl]indazole-3-carboxamide (580 mg, 40% yield) as a white solid. LCMS (ES, m/z): [M+H]⁺: 683.

### Part VII—Synthesis of 6-[3-[5-Cyano-2-(difluoromethoxy)pyridine-3-sulfonamido]-2,6-difluorophenyl]-7-fluoro-N-methyl-1H-indazole-3-carboxamide

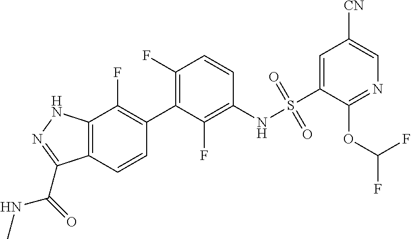 [SMILES: CNC(=O)c1n[nH]c2c(F)c(-c3c(F)ccc(NS(=O)(=O)c4cc(C#N)cnc4OC(F)F)c3F)ccc12]

A solution of 6-[3-[5-cyano-2-(difluoromethoxy)pyridine-3-sulfonamido]-2,6-difluorophenyl]-7-fluoro-N-methyl-1-[[2-(trimethylsilyl)ethoxy]methyl]indazole-3-carboxamide (200 mg, 0.3 mmol, 1 equiv) and TFA (10 mL) in DCM (20 mL) was stirred for 4 hours at room temperature. The reaction was concentrated under vacuum. The residue was dissolved in 7 M NH₃ solution in MeOH (50 mL) and stirred for 30 minutes. The resulting solution was concentrated under reduced pressure. The residue was dissolved in EA (50 mL). The organic phase was washed with H₂O (2×20 mL), dried (Na2SO4) and concentrated under reduced pressure. The residue was purified by prep-HPLC: Welch Vltimate XB-C18 column, 50×250 mm, 10 μm, mobile phase: water (0.1% FA) and CH₃CN; Gradient: 40% B to 80% in 8 min; Flow rate: 90 mL/min; Detector, 220 nm. 6-[3-[5-Cyano-2-(difluoromethoxy)pyridine-3-sulfonamido]-2,6-difluorophenyl]-7-fluoro-N-methyl-1H-indazole-3-carboxamide (37 mg, 23% yield) was obtained as a white solid. LCMS (ES, m/z): [M+H]⁺: 553. ¹H NMR (300 MHz, DMSO-d₆) δ 14.27 (s, 1H), 8.94 (s, 1H), 8.65 (s, 1H), 8.49 (d, J=4.9 Hz, 1H), 8.04 (d, J=8.8 Hz, 1H), 7.41 (s, 1H), 7.18-7.07 (m, 1H), 2.84 (d, J=4.7 Hz, 3H).

### Example 47—Synthesis of 6-[3-(5-chloro-2-methoxypyridine-3-sulfonamido)-2,6-difluorophenyl]-7-fluoro-N,4-dimethyl-1H-indazole-3-carboxamide

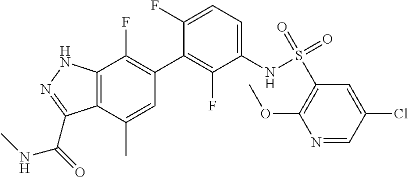 [SMILES: CNC(=O)c1n[nH]c2c(F)c(-c3c(F)ccc(NS(=O)(=O)c4cc(Cl)cnc4OC)c3F)cc(C)c12]

### Part I—Synthesis of (2E)-N-(2-fluoro-5-methylphenyl)-2-(N-hydroxyimino)acetamide

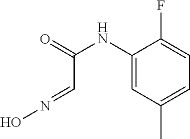

To a stirred solution of Na₂SO₄ (55 g, 384 mmol, 8 equiv) and chloral hydrate (8.8 g, 52.8 mmol, 1.1 equiv) in H₂O (200 mL) was added 2-fluoro-5-methylaniline (6 g, 48 mmol, 1 equiv) in HCl (4 mL, 112 mmol, 2.8 equiv) dropwise at room temperature. To the above mixture was added NH₂OH.HCl (3.7 g, 52.8 mmol, 1.1 equiv) in H₂O (35 mL) dropwise, maintaining ambient temperature and this was stirred for an additional 1 h at 100° C. The mixture was allowed to cool and the precipitated solids were collected by filtration and washed with water (3×100 mL). The resulting solid was dried under infrared light to give (2E)-N-(2-fluoro-5-methylphenyl)-2-(N-hydroxyimino)acetamide (7.5 g, 80% yield) as a yellow solid. LCMS (ES, m/z): [M+H]⁺: 197.

### Part II—Synthesis of 7-fluoro-4-methyl-1H-indole-2,3-dione

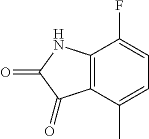

To H₂SO₄ (75 mL) was added (2E)-N-(2-fluoro-5-methylphenyl)-2-(N-hydroxyimino)acetamide (7.5 g, 38 mmol, 1 equiv) in portions at room temperature. The resulting mixture was stirred for 2 h at 70° C. The mixture was cooled and quenched with water/ice. The precipitated solids were collected by filtration and washed with water (3×100 mL). The resulting solid was dried under infrared light to give 7-fluoro-4-methyl-1H-indole-2,3-dione (5.1 g, 60% yield) as a yellow solid. LCMS (ES, m/z): [M+H]⁺: 180.

### Part III—Synthesis of 7-fluoro-4-methyl-1H-indazole-3-carboxylic acid

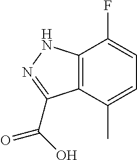

The substituted 7-fluoro-4-methyl-1H-indole-2,3-dione (1.7 g, 9.5 mmol, 1 equiv) was treated with 1M NaOH (3 mL, 30 mmol, 3.1 equiv) and was heated at 50° C. for 30 min. The solution was allowed to cool to room temperature and was stirred for 1 h. The reaction mixture was cooled to 0° C. and was treated with a 0° C. solution of NaNO₂ (655 mg, 9.5 mmol, 1 equiv) in water (2 mL). This solution was added over 15 min through a cannula submerged below the surface of a vigorously stirred solution of H₂SO₄ (0.6 mL) in water (12 mL) at 0° C. After an additional 10 min, a solution of SnCl₂.2H₂O (5.2 g, 22.8 mmol, 2.4 equiv) in concentrated HCl (20 mL) was added and the reaction mixture was stirred for 60 min. The precipitated solids were isolated by filtration, washed with water, and dried under infrared light to give 7-fluoro-4-methyl-1H-indazole-3-carboxylic acid (600 mg, 33%) as a yellow solid. LCMS (ES, m/z): [M+H]⁻: 193.

### Part IV—Synthesis of methyl 7-fluoro-4-methyl-1H-indazole-3-carboxylate

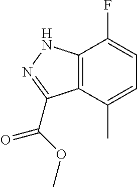

To a stirred solution of 7-fluoro-4-methyl-1H-indazole-3-carboxylic acid (50 mg, 0.26 mmol, 1 equiv) in DCM (6 mL) was added oxalyl chloride (65 mg, 0.52 mmol, 2 equiv) dropwise at room temperature. The resulting mixture was stirred for 2 h then concentrated under reduced pressure. The residue was diluted with DCM (6 mL), and MeOH (0.25 mL) added dropwise at 0-5° C. The resulting mixture was stirred for additional 1 h at room temperature, then diluted with water (5 mL). This was extracted with CH₂Cl₂ (2×10 mL). The combined organic layers were washed with water and brine, dried over anhydrous Na₂SO₄. After filtration, the filtrate was concentrated under reduced pressure. The residue was purified by silica gel column chromatography, eluted with EtOAc/PE (0-50%) to afford methyl 7-fluoro-4-methyl-1H-indazole-3-carboxylate (100 mg, 16%) as a yellow solid. LCMS (ES, m/z): [M+H]⁺: 209.

### Part V—Synthesis of methyl 7-fluoro-4-methyl-1-[[2-(trimethylsilyl)ethoxy] methyl]indazole-3-carboxylate

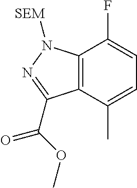 [SMILES: COC(=O)c1nn(COCC[Si](C)(C)C)c2c(F)ccc(C)c12]

To a stirred solution of methyl 7-fluoro-4-methyl-1H-indazole-3-carboxylate (100 mg, 0.48 mmol, 1 equiv) in THE (1 mL) was added NaH (38 mg, 0.96 mmol, 2 equiv, 60%) in portions at 0° C. under nitrogen atmosphere. The resulting mixture was stirred for 10 min at 0° C. and SEMCl (120 mg, 0.72 mmol, 1.5 equiv) was then added dropwise at 0° C. The reaction was stirred to ambient temperature over 2 hrs, then quenched with water at 0° C. The resulting mixture was extracted with EtOAc (3×20 mL). The combined organic layers were washed with water and brine, dried over anhydrous Na₂SO₄. After filtration, the filtrate was concentrated under reduced pressure. The residue was purified by silica gel column chromatography, eluted with EtOAc/PE (0-50%) to afford methyl 7-fluoro-4-methyl-1-[[2-(trimethylsilyl)ethoxy] methyl]indazole-3-carboxylate (70 mg, 43%) as a yellow solid. LCMS (ES, m/z): [M+H]⁺: 339.

### Part VI—Synthesis of 7-fluoro-4-methyl-6-(4,4,5,5-tetramethyl-1,3,2-dioxaborolan-2-yl)-1-[[2-(trimethylsilyl)ethoxy]methyl]indazole-3-carboxylate

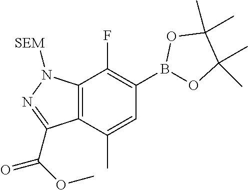 [SMILES: COC(=O)c1nn(COCC[Si](C)(C)C)c2c(F)c(B3OC(C)(C)C(C)(C)O3)cc(C)c12]

A solution of bis(pinacolato)diboron (550 mg, 2.2 mmol, 6.7 equiv), 4,4′-di-tert-butyl-2,2′-bipyridine (5 mg, 0.015 mmol, 0.05 equiv) and bis(1,5-cyclooctadiene,(Z,Z)-)dimethyl-2,4-dioxa-1,3-diiridabicyclo [1.1.0]butane-2,4-diium (11 mg, 0.015 mmol, 0.05 equiv) in THE (2.2 mL) was stirred for 10 min at 50° C. under nitrogen atmosphere. To the above mixture was added methyl 7-fluoro-4-methyl-1-[[2-(trimethylsilyl)ethoxy]methyl]indazole-3-carboxylate (110 mg, 0.3 mmol, 1 equiv) in THE dropwise at 50° C. This was stirred for additional 16 h at 50° C. The resulting mixture was concentrated under reduced pressure. The residue was purified by silica gel column chromatography, eluting with EtOAc/PE (0-20%) to afford methyl 7-fluoro-4-methyl-6-(4,4,5,5-tetramethyl-1,3,2-dioxaborolan-2-yl)-1-[[2-(trimethylsilyl)ethoxy]methyl]indazole-3-carboxylate (113 mg, 74%) as a yellow oil. LCMS (ES, m/z): [M+H]⁺: 465.

### Part VII—Synthesis of methyl 6-[3-(5-chloro-2-methoxypyridine-3-sulfonamido)-2,6-difluorophenyl]-7-fluoro-4-methyl-1-[[2-(trimethylsilyl)ethoxy]methyl]indazole-3-carboxylate

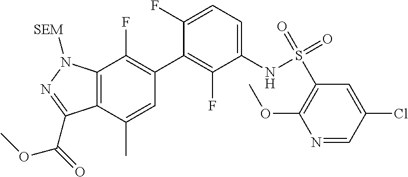 [SMILES: COC(=O)c1nn(COCC[Si](C)(C)C)c2c(F)c(-c3c(F)ccc(NS(=O)(=O)c4cc(Cl)cnc4OC)c3F)cc(C)c12]

To a mixture of methyl 7-fluoro-4-methyl-6-(4,4,5,5-tetramethyl-1,3,2-dioxaborolan-2-yl)-1-[[2-(trimethylsilyl) ethoxy]methyl]indazole-3-carboxylate (110 mg, 0.24 mmol, 1 equiv) and N-(3-bromo-2,4-difluorophenyl)-5-chloro-2-methoxypyridine-3-sulfonamide (98 mg, 0.24 mmol, 1 equiv) in dioxane (1 mL) and H₂O (0.1 mL) were added S-phos (19 mg, 0.048 mmol, 0.2 equiv), SPhosPd Gen₃ (18.5 mg, 0.024 mmol, 0.1 equiv) and K₂CO₃ (50 mg, 0.36 mmol, 1.5 equiv). After stirring for 16 h at 100° C. under a nitrogen atmosphere, the resulting mixture was concentrated under reduced pressure. The residue was purified by silica gel column chromatography, eluted with EtOAc/PE (0-25%) to afford methyl 6-[3-(5-chloro-2-methoxypyridine-3-sulfonamido)-2,6-difluorophenyl]-7-fluoro-4-methyl-1-[[2-(trimethylsilyl)ethoxy]methyl]indazole-3-carboxylate (58 mg, 36%) as a light yellow oil. LCMS (ES, m/z): [M+H]⁺: 671.

### Part VIII—Synthesis of 6-[3-(5-chloro-2-methoxypyridine-3-sulfonamido)-2,6-difluorophenyl]-7-fluoro-N,4-dimethyl-1-[[2-(trimethylsilyl) ethoxy]methyl]indazole-3-carboxamide

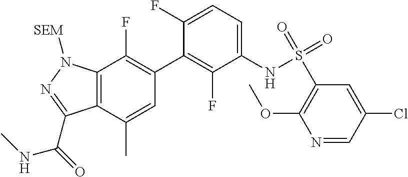 [SMILES: CNC(=O)c1nn(COCC[Si](C)(C)C)c2c(F)c(-c3c(F)ccc(NS(=O)(=O)c4cc(Cl)cnc4OC)c3F)cc(C)c12]

A solution of methyl 6-[3-(5-chloro-2-methoxypyridine-3-sulfonamido)-2,6-difluorophenyl]-7-fluoro-4-methyl-1-[[2-(trimethylsilyl)ethoxy]methyl]indazole-3-carboxylate (70 mg, 0.1 mmol, 1 equiv) in THE (0.5 mL) and CH₃NH₂ (0.5 mL, 30% in H₂O) was stirred for 5 h at room temperature. The resulting mixture was concentrated under vacuum to give 6-[3-(5-chloro-2-methoxypyridine-3-sulfonamido)-2,6-difluorophenyl]-7-fluoro-N,4-dimethyl-1-[[2-(trimethylsilyl) ethoxy]methyl]indazole-3-carboxamide (66 mg, crude) as a light yellow oil. LCMS (ES, m/z): [M+H]⁺: 670.

### Part IX—Synthesis of 6-[3-(5-chloro-2-methoxypyridine-3-sulfonamido)-2,6-difluorophenyl]-7-fluoro-N,4-dimethyl-1H-indazole-3-carboxamide

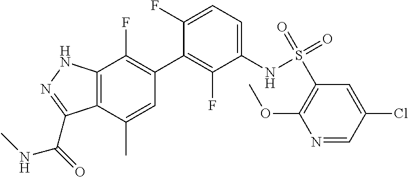 [SMILES: CNC(=O)c1n[nH]c2c(F)c(-c3c(F)ccc(NS(=O)(=O)c4cc(Cl)cnc4OC)c3F)cc(C)c12]

A solution of 6-[3-(5-chloro-2-methoxypyridine-3-sulfonamido)-2,6-difluorophenyl]-7-fluoro-N,4-dimethyl-1-[[2-(trimethylsilyl)ethoxy]methyl]indazole-3-carboxamide (56 mg, 0.084 mmol, 1 equiv) in DCM (0.3 mL) and TFA (0.1 mL) was stirred for 3 h at room temperature. The resulting mixture was concentrated under vacuum. The residue was dissolved in NH₃ (0.5 mL, 7M in MeOH). The resulting mixture was stirred for 2 h at room temperature, then concentrated under vacuum. The crude product was purified by Prep-HPLC with the following conditions Column: welch Vltimate XB-C18, 50×250 mm, 10 μm, mobile phase: 90 mL/min, Mobile Phase A: 0.1% FA in Water, Mobile Phase B: ACN (30% up to 50% in 12 min) to afford 6-[3-(5-chloro-2-methoxypyridine-3-sulfonamido)-2,6-difluorophenyl]-7-fluoro-N,4-dimethyl-1H-indazole-3-carboxamide (5.7 mg, 12.6%) as a white solid. LCMS (ES, m/z): [M−H]⁻: 538. ¹H NMR (300 MHz, DMSO-d₆) δ 14.14 (s, 1H), 10.45 (s, 1H), 8.43-8.51 (m, 2H), 8.05 (d, J=2.4 Hz, 1H), 7.38 (s, 1H), 7.17 (s, 1H), 6.83 (d, J=5.7 Hz, 1H), 3.88 (s, 3H), 2.82 (s, 3H), 2.67 (s, 3H).

### Example 48—Synthesis of 6-[3-(5-Chloro-2-methoxypyridine-3-sulfonamido)-2,6-difluorophenyl]-7-fluoro-N-(2-methylpropyl)-1H-indazole-3-carboxamide

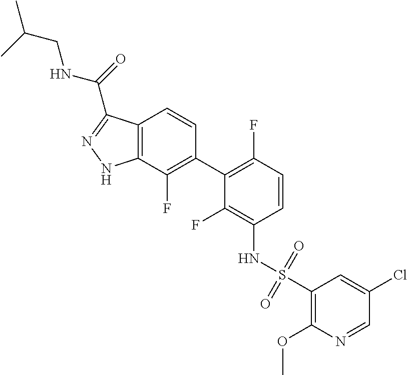 [SMILES: COc1ncc(Cl)cc1S(=O)(=O)Nc1ccc(F)c(-c2ccc3c(C(=O)NCC(C)C)n[nH]c3c2F)c1F]

### Part I—Synthesis of methyl 7-fluoro-6-(4,4,5,5-tetramethyl-1,3,2-dioxaborolan-2-yl)-1-[[2-(trimethylsilyl)ethoxy]methyl]indazole-3-carboxylate

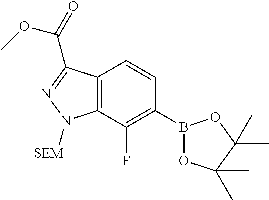

Into a 500-mL round-bottom flask purged and maintained with an inert atmosphere of nitrogen, was placed methyl-6-bromo-7-fluoro-1-[[2-(trimethylsilyl)ethoxy]methyl]indazole-3-carboxylate (See Example 4) (23 g, 57 mmol, 1 equiv), bis(pinacolato)diboron (29 g, 114 mmol, 2 equiv), Pd(dppf)Cl₂ (8.4 g, 11.4 mmol, 0.20 equiv), KOAc (8.4 g, 86 mmol, 1.5 equiv) and dioxane (300 mL). The resulting solution was stirred for overnight at 100 degrees C., then cooled and filtered. The filter cake was washed twice with dioxane (10 ml), and the combined filtrate was concentrated. The residue was dissolved in 200 mL of cyclohexane and the suspension formed was filtered. Concentration of the filtrate gave crude methyl 7-fluoro-6-(4,4,5,5-tetramethyl-1,3,2-dioxaborolan-2-yl)-1-[[2-(trimethylsilyl)ethoxy]methyl]indazole-3-carboxylate (15.2 g, 59%) as brown oil. LCMS (ES, m/z): 451[M+H]⁺.

### Part II—Synthesis of methyl 6-(3-amino-2,6-difluorophenyl)-7-fluoro-1-[[2-(trimethylsilyl)ethoxy]methyl] indazole-3-carboxylate

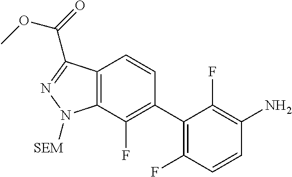 [SMILES: COC(=O)c1nn(COCC[Si](C)(C)C)c2c(F)c(-c3c(F)ccc(N)c3F)ccc12]

Into a 250-mL round-bottom flask purged and maintained with an inert atmosphere of nitrogen, was placed methyl 7-fluoro-6-(4,4,5,5-tetramethyl-1,3,2-dioxaborolan-2-yl)-1-[[2-(trimethylsilyl)ethoxy]methyl]indazole-3-carboxylate (15.2 g, 34 mmol, 1 equiv), 3-bromo-2,4-difluoroaniline (5.6 g, 27 mmol, 0.8 equiv), SPhos Pd Gen.3 (2.63 g, 3.4 mmol, 0.1 equiv), K₂CO₃ (7.0 g, 51 mmol, 1.5 equiv), SPhos (2.77 g, 6.8 mmol, 0.2 equiv), dioxane (100 mL) and H₂O (25 mL). The solution was stirred for 1 hr at 100 degrees C., then cooled and diluted with 50 mL of H₂O. The resulting mixture was extracted with 3×150 mL of ethyl acetate, and the extracts dried over anhydrous sodium sulfate. The extracts were concentrated and the residue applied to a silica gel column, eluting with ethyl acetate/petroleum ether (1:2). Concentration of the appropriate fractions gave methyl 6-(3-amino-2,6-difluorophenyl)-7-fluoro-1-[[2-(trimethylsilyl)ethoxy]methyl]indazole-3-carboxylate (10.2 g, 67% yield) as a brown oil. LCMS (ES, m/z): 452[M+H]⁺.

### Part III—Synthesis of methyl 6-[3-(5-chloro-2-methoxypyridine-3-sulfonamido)-2,6-difluorophenyl]-7-fluoro-1-[[2-(trimethylsilyl) ethoxy] methyl] indazole-3-carboxylate

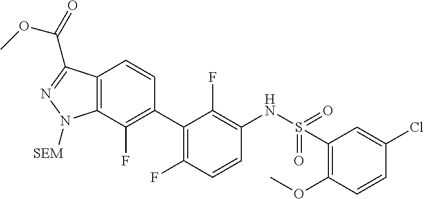 [SMILES: COC(=O)c1nn(COCC[Si](C)(C)C)c2c(F)c(-c3c(F)ccc(NS(=O)(=O)c4cc(Cl)ccc4OC)c3F)ccc12]

Into a 250-mL round-bottom flask, was placed methyl 6-(3-amino-2,6-difluorophenyl)-7-fluoro-1-[[2-(trimethylsilyl) ethoxy] methyl] indazole-3-carboxylate (10.2 g, 23 mmol, 1 equiv), CH₂Cl₂ (150 mL) and pyridine (17.9 g, 226 mmol, 10 equiv). This was followed by the addition of 2-methoxy-5-methylpyridine-3-sulfonyl chloride (7.51 g, 34 mmol, 1.5 equiv) at 0 degrees C. The resulting solution was stirred for 1 hr at low temperature, then diluted with 100 mL of H₂O. Extraction with 3×100 mL of dichloromethane and concentration gave methyl 6-[3-(5-chloro-2-methoxypyridine-3-sulfonamido)-2,6-difluorophenyl]-7-fluoro-1-[[2-(trimethylsilyl) ethoxy] methyl] indazole-3-carboxylate (13.1 g, 88% yield) as a brown solid. LCMS (ES, m/z):658 [M+H]⁺.

### Part IV—Synthesis of methyl 6-[3-(5-chloro-2-methoxypyridine-3-sulfonamido)-2,6-difluorophenyl]-7-fluoro-1H-indazole-3-carboxylate

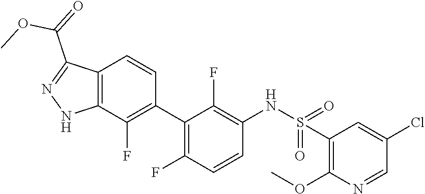 [SMILES: COC(=O)c1n[nH]c2c(F)c(-c3c(F)ccc(NS(=O)(=O)c4cc(Cl)cnc4OC)c3F)ccc12]

Into a 250-mL round-bottom flask, was placed methyl 6-[3-(5-chloro-2-methoxypyridine-3-sulfonamido)-2,6-difluorophenyl]-7-fluoro-1-[[2-(trimethylsilyl)ethoxy]methyl]indazole-3-carboxylate (13.1 g, 20 mmol, 1 equiv) and CH₂Cl₂ (100 mL). TFA (50 mL) was added dropwise at 0 degrees C. over 20 min. The resulting solution was stirred for 2 hr at 0 degrees C., then quenched by the addition of 200 mL of water/ice. The pH was adjusted to 10 with saturated aqueous NaHCO₃ and the mixture extracted with 3×150 mL of dichloromethane. The extracts were dried over anhydrous sodium sulfate and concentrated. The residue was applied to a silica gel column, eluting with ethyl acetate/petroleum ether (1:1). The appropriate fractions were combined and concentrated to give methyl 6-[3-(5-chloro-2-methoxypyridine-3-sulfonamido)-2,6-difluorophenyl]-7-fluoro-1H-indazole-3-carboxylate (9.82 g) as an off-white solid. LCMS (ES, m/z):527 [M+H]⁺.

### Part V—Synthesis of 6-[3-(5-chloro-2-methoxypyridine-3-sulfonamido)-2,6-difluorophenyl]-7-fluoro-1H-indazole-3-carboxylic acid

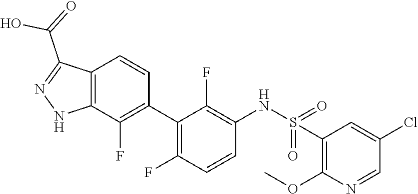 [SMILES: COc1ncc(Cl)cc1S(=O)(=O)Nc1ccc(F)c(-c2ccc3c(C(=O)O)n[nH]c3c2F)c1F]

Into a 250-mL round-bottom flask, was placed methyl 6-[3-(5-chloro-2-methoxypyridine-3-sulfonamido)-2,6-difluorophenyl]-7-fluoro-1H-indazole-3-carboxylate (9.7 g, 18.4 mmol, 1 equiv), CH₃OH (30 mL), THE (30 mL), H₂O (30 mL) and LiOH (0.88 g, 37 mmol, 2 equiv). The resulting solution was stirred for 3 hr at 50 degrees C., then concentrated. The pH was adjusted to 5 with 2M HCl, and the solid formed removed by filtration. Drying under vacuum gave 6-[3-(5-chloro-2-methoxypyridine-3-sulfonamido)-2,6-difluorophenyl]-7-fluoro-1H-indazole-3-carboxylic acid (8.2 g, 87% yield) as an off-white solid. LCMS (ES, m/z):512 [M+H]⁺.

### Part VI—Synthesis of 6-[3-(5-Chloro-2-methoxypyridine-3-sulfonamido)-2,6-difluorophenyl]-7-fluoro-N-(2-methylpropyl)-1H-indazole-3-carboxamide

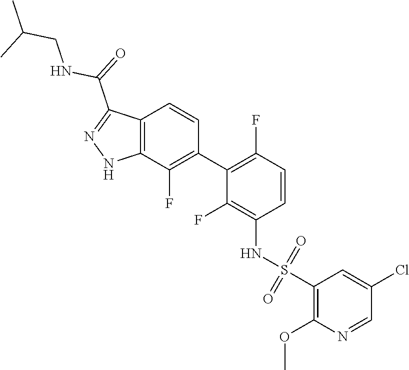 [SMILES: COc1ncc(Cl)cc1S(=O)(=O)Nc1ccc(F)c(-c2ccc3c(C(=O)NCC(C)C)n[nH]c3c2F)c1F]

To a stirred solution of 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv) in CH₃CN (3 mL) was added HATU (89 mg, 0.2 mmol, 1.2 equiv), DIEA (76 mg, 0.59 mmol, 3 equiv) and isobutylamine (29 mg, 0.4 mmol, 2 equiv) at room temperature. The resulting mixture was stirred for 12 h at 50 degrees C., then cooled and filtered. The filtrate was concentrated and purified by Prep-HPLC with the following conditions: Column, Welch-Xtimate, 30*150 mm, 10 μm; mobile phase, Water (0.1% FA) and CH3CN (38% CH3CN up to 78% in 6 min); Detector, 254 nm & 220 nm. 6-[3-(5-Chloro-2-methoxypyridine-3-sulfonamido)-2,6-difluorophenyl]-7-fluoro-N-(2-methylpropyl)-1H-indazole-3-carboxamide (23 mg, 21% yield) was isolated as an off-white solid. LCMS (ES, m/z): [M+H]⁺: 568. ¹H NMR (300 MHz, DMSO-d₆) δ 14.31 (s, 1H), 10.46 (s, 1H), 8.63-8.43 (m, 2H), 8.18-7.90 (m, 2H), 7.60-7.41 (m, 1H), 7.41-7.21 (m, 1H), 7.21-6.98 (m, 1H), 3.91 (d, J=1.9 Hz, 3H), 3.15 (t, J=6.6 Hz, 2H), 2.03-1.75 (m, 1H), 0.91 (d, J=6.7 Hz, 6H).

### Example 49—Synthesis of 6-(3-((5-Chloro-2-methoxypyridine)-3-sulfonamido)-2,6-difluorophenyl)-N-ethyl-7-fluoro-1H-indazole-3-carboxamide

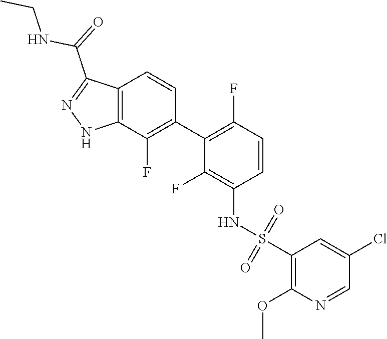 [SMILES: CCNC(=O)c1n[nH]c2c(F)c(-c3c(F)ccc(NS(=O)(=O)c4cc(Cl)cnc4OC)c3F)ccc12]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), ethylamine hydrochloride (32 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The reaction mixture was purified by Prep-HPLC with the following conditions: Column, T3, 19*150 mm, 5 μm; mobile phase, Water (0.1% FA) and CH₃CN (40% CH₃CN up to 60% in 6 min); Detector, 254 nm & 220 nm. 6-(3-((5-Chloro-2-methoxypyridine)-3-sulfonamido)-2,6-difluorophenyl)-N-ethyl-7-fluoro-1H-indazole-3-carboxamide (53 mg, 50% yield) was isolated as a white solid. LCMS (ES, m/z): [M+H]⁺: 540. ¹H NMR (300 MHz, DMSO-d₆) δ 14.31 (s, 1H), 10.45 (s, 1H), 8.56 (t, J=6.0 Hz, 1H), 8.51 (d, J=2.6 Hz, 1H), 8.12-8.01 (m, 2H), 7.55-7.39 (m, 1H), 7.37-7.21 (m, 1H), 7.21-7.05 (m, 1H), 3.91 (s, 3H), 3.41-3.34 (m, 2H), 1.16 (t, J=7.1 Hz, 3H).

### Example 50—Synthesis of 6-[3-(5-Chloro-2-methoxypyridine-3-sulfonamido)-2,6-difluorophenyl]-7-fluoro-N-(1-methyl-6-oxopiperidin-3-yl)-1H-indazole-3-carboxamide

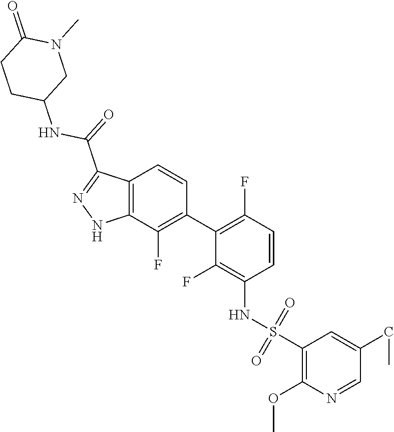 [SMILES: CCc1cnc(OC)c(S(=O)(=O)Nc2ccc(F)c(-c3ccc4c(C(=O)NC5CCC(=O)N(C)C5)n[nH]c4c3F)c2F)c1]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), 5-amino-1-methylpiperidin-2-one (50 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The reaction mixture was purified by Prep-HPLC with the following conditions: Column, Xselect Fluoro-Phenyl, 19*150 mm, 5 μm; mobile phase, Water (0.1% FA) and CH₃CN (33% CH₃CN up to 53% in 7 min); Detector, 254 nm & 220 nm. 6-[3-(5-Chloro-2-methoxypyridine-3-sulfonamido)-2,6-difluorophenyl]-7-fluoro-N-(1-methyl-6-oxopiperidin-3-yl)-1H-indazole-3-carboxamide (40.7 mg, 34% yield) was isolated as a white solid. LCMS (ES, m/z): [M+H]⁺: 623. ¹H NMR (300 MHz, DMSO-d₆) δ 14.40 (s, 1H), 9.95 (br, s, 1H), 8.69 (d, J=7.8 Hz, 1H), 8.51 (d, J=2.6 Hz, 1H), 8.19-7.97 (m, 2H), 7.55-7.41 (m, 1H), 7.38-7.24 (m, 1H), 7.24-7.10 (m, 1H), 4.49-4.22 (m, 1H), 3.91 (s, 3H), 3.53-3.42 (m, 1H), 3.42-3.36 (m, 1H), 2.82 (s, 3H), 2.44-2.32 (m, 2H), 2.15-1.86 (m, 2H).

### Example 51—Synthesis of N-(1-acetylpyrrolidin-3-yl)-6-[3-(5-chloro-2-methoxypyridine-3-sulfonamido)-2,6-difluorophenyl]-7-fluoro-1H-indazole-3-carboxamide

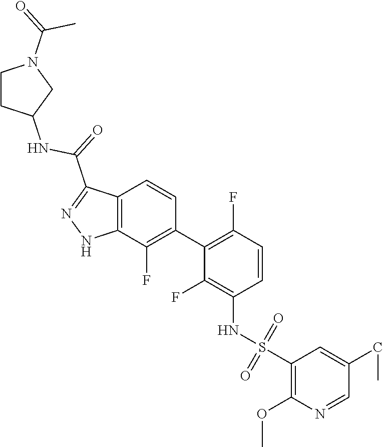 [SMILES: CCc1cnc(OC)c(S(=O)(=O)Nc2ccc(F)c(-c3ccc4c(C(=O)NC5CCN(C(C)=O)C5)n[nH]c4c3F)c2F)c1]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), 1-(3-aminopyrrolidin-1-yl)ethanone (50 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, T3, 19*150 mm, 5 μm; mobile phase, Water (0.1% FA) and CH₃CN (40% CH₃CN up to 57% in 6 min); Detector, 254 nm & 220 nm. N-(1-acetylpyrrolidin-3-yl)-6-[3-(5-chloro-2-methoxypyridine-3-sulfonamido)-2,6-difluorophenyl]-7-fluoro-1H-indazole-3-carboxamide (49 mg, 41% yield) was isolated as a white solid. LCMS (ES, m/z): [M+H]⁺: 623. ¹H NMR (300 MHz, DMSO-d₆) δ 14.37 (d, J=4.2 Hz, 1H), 10.46 (s, 1H), 8.91-8.66 (m, 1H), 8.51 (d, J=2.6 Hz, 1H), 8.19-7.94 (m, 2H), 7.59-7.38 (m, 1H), 7.38-7.22 (m, 1H), 7.22-7.08 (m, 1H), 4.73-4.42 (m, 1H), 3.91 (s, 3H), 3.83-3.40 (m, 3H), 3.40-3.33 (m, 1H), 2.34-2.01 (m, 2H), 1.95 (d, J=2.7 Hz, 3H).

### Example 52—Synthesis of 6-[3-(5-Chloro-2-methoxypyridine-3-sulfonamido)-2,6-difluorophenyl]-7-fluoro-N-(2-hydroxypropyl)-1H-indazole-3-carboxamide

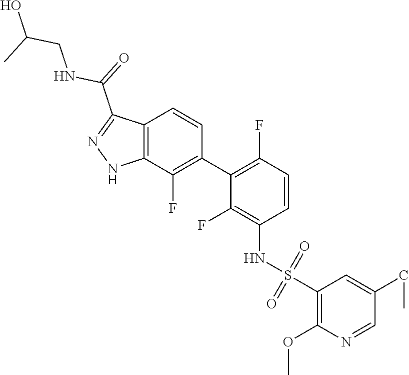 [SMILES: CCc1cnc(OC)c(S(=O)(=O)Nc2ccc(F)c(-c3ccc4c(C(=O)NCC(C)O)n[nH]c4c3F)c2F)c1]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), 1-amino-2-propanol (29 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, Xselect Fluoro-Phenyl, 19*150 mm, 5 μm; mobile phase, Water (0.1% FA) and CH₃CN (33% CH₃CN up to 53% in 7 min); Detector, 254 nm & 220 nm. 6-[3-(5-Chloro-2-methoxypyridine-3-sulfonamido)-2,6-difluorophenyl]-7-fluoro-N-(2-hydroxypropyl)-1H-indazole-3-carboxamide (20 mg, 18% yield) was isolated as a white solid. LCMS (ES, m/z): [M+H]⁺: 570. ¹H NMR (300 MHz, DMSO-d₆) δ 14.35 (s, 1H), 10.36 (br, s, 1H), 8.51 (d, J=2.6 Hz, 1H), 8.36-8.23 (m, 1H), 8.12-8.00 (m, 2H), 7.54-7.39 (m, 1H), 7.35-7.22 (m, 1H), 7.20-7.08 (m, 1H), 4.82 (d, J=4.8 Hz, 1H), 3.91 (s, 3H), 3.88-3.78 (m, 1H), 3.41-3.34 (m, 1H), 3.27-3.16 (m, 1H), 1.10 (d, J=6.2 Hz, 3H).

### Example 53—Synthesis of 6-[3-(5-Chloro-2-methoxypyridine-3-sulfonamido)-2,6-difluorophenyl]-7-fluoro-N-(1-hydroxypropan-2-yl)-1H-indazole-3-carboxamide

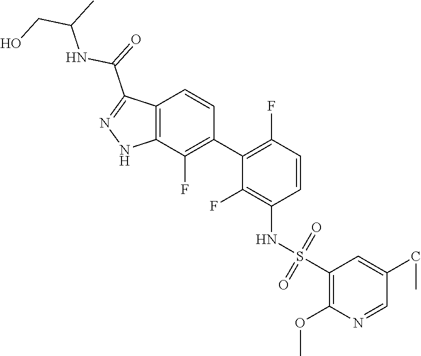 [SMILES: CCc1cnc(OC)c(S(=O)(=O)Nc2ccc(F)c(-c3ccc4c(C(=O)NC(C)CO)n[nH]c4c3F)c2F)c1]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), 2-aminopropanol (29 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, T3, 19*150 mm, 5 μm; mobile phase, Water (0.1% FA) and CH₃CN (58% CH₃CN up to 73% in 7 min); Detector, 254 nm & 220 nm. 6-[3-(5-Chloro-2-methoxypyridine-3-sulfonamido)-2,6-difluorophenyl]-7-fluoro-N-(1-hydroxypropan-2-yl)-1H-indazole-3-carboxamide (30 mg, 27% yield) was isolated as a white solid. LCMS (ES, m/z): [M+H]⁺: 570. ¹H NMR (300 MHz, DMSO-d₆) δ 14.33 (s, 1H), 10.45 (s, 1H), 8.51 (d, J=2.6 Hz, 1H), 8.15-7.99 (m, 3H), 7.54-7.39 (m, 1H), 7.35-7.23 (m, 1H), 7.19-7.08 (m, 1H), 4.81 (t, J=5.6 Hz, 1H), 4.18-4.02 (m, 1H), 3.91 (s, 3H), 3.59-3.39 (m, 2H), 1.22-1.15 (m, 3H).

### Example 54—Synthesis of 6-[3-(5-Chloro-2-methoxypyridine-3-sulfonamido)-2,6-difluorophenyl]-7-fluoro-N-(2-oxopyrrolidin-3-yl)-1H-indazole-3-carboxamide

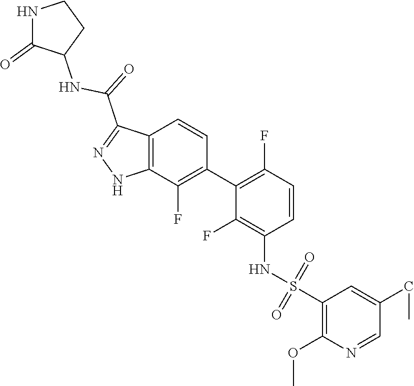 [SMILES: CCc1cnc(OC)c(S(=O)(=O)Nc2ccc(F)c(-c3ccc4c(C(=O)NC5CCNC5=O)n[nH]c4c3F)c2F)c1]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), 3-aminopyrrolidin-2-one (39 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, Xselect Fluoro-Phenyl, 19*150 mm, 5 μm; mobile phase, Water (0.1% FA) and CH₃CN (30% CH3CN up to 50% in 7 min); Detector, 254 nm & 220 nm. 6-[3-(5-Chloro-2-methoxypyridine-3-sulfonamido)-2,6-difluorophenyl]-7-fluoro-N-(2-oxopyrrolidin-3-yl)-1H-indazole-3-carboxamide (26 mg, 22% yield) was isolated as a white solid. LCMS (ES, m/z): [M+H]+: 595. ¹H NMR (300 MHz, DMSO-d₆) δ 14.39 (s, 1H), 8.67 (d, J=8.4 Hz, 1H), 8.49 (d, J=2.6 Hz, 1H), 8.11-8.02 (m, 2H), 7.84 (s, 1H), 7.51-7.39 (m, 1H), 7.33-7.22 (m, 1H), 7.21-7.10 (m, 1H), 4.68-4.51 (m, 1H), 3.90 (s, 3H), 3.27-3.21 (m, 2H), 2.44-2.31 (m, 1H), 2.24-2.09 (m, 1H).

### Example 55—Synthesis of 6-[3-(5-Chloro-2-methoxypyridine-3-sulfonamido)-2,6-difluorophenyl]-7-fluoro-N-(oxan-3-yl)-1H-indazole-3-carboxamide

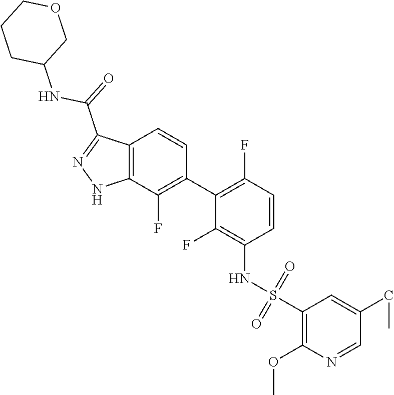 [SMILES: CCc1cnc(OC)c(S(=O)(=O)Nc2ccc(F)c(-c3ccc4c(C(=O)NC5CCCOC5)n[nH]c4c3F)c2F)c1]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), oxan-3-amine (40 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, Welch-Xtimate, 30*150 mm, 10 μm; mobile phase, Water (0.1% FA) and CH₃CN (38% CH₃CN up to 78% in 6 min); Detector, 254 nm & 220 nm. 6-[3-(5-Chloro-2-methoxypyridine-3-sulfonamido)-2,6-difluorophenyl]-7-fluoro-N-(oxan-3-yl)-1H-indazole-3-carboxamide (33 mg, 29% yield) was isolated as a white solid. LCMS (ES, m/z): [M+H]⁺: 596. ¹H NMR (300 MHz, DMSO-d₆) δ 14.36 (s, 1H), 10.45 (s, 1H), 8.51 (d, J=2.6 Hz, 1H), 8.30 (d, J=8.3 Hz, 1H), 8.17-7.94 (m, 2H), 7.54-7.37 (m, 1H), 7.37-7.23 (m, 1H), 7.23-7.00 (m, 1H), 4.09-3.94 (m, 1H), 3.91 (s, 3H), 3.85-3.69 (m, 2H), 3.39-3.34 (m, 1H), 3.30-3.24 (m, 1H), 2.01-1.86 (m, 1H), 1.84-1.50 (m, 3H).

### Example 56—Synthesis of 6-[3-(5-Chloro-2-methoxypyridine-3-sulfonamido)-2,6-difluorophenyl]-7-fluoro-N-(sec-butyl)-1H-indazole-3-carboxamide

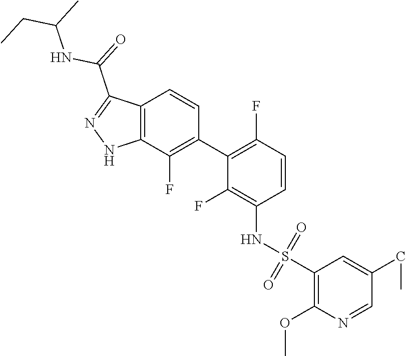 [SMILES: CCc1cnc(OC)c(S(=O)(=O)Nc2ccc(F)c(-c3ccc4c(C(=O)NC(C)CC)n[nH]c4c3F)c2F)c1]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), sec-butylamine (29 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, Xselect Fluoro-Phenyl, 19*150 mm, 5 μm; mobile phase, Water (0.1% FA) and CH₃CN (55% CH₃CN up to 75% in 7 min); Detector, 254 nm & 220 nm. 6-[3-(5-Chloro-2-methoxypyridine-3-sulfonamido)-2,6-difluorophenyl]-7-fluoro-N-(sec-butyl)-1H-indazole-3-carboxamide (41 mg, 37% yield) was isolated as a white solid. LCMS (ES, m/z): [M+H]⁺: 568. ¹H NMR (300 MHz, DMSO-d₆) δ 14.30 (s, 1H), 10.43 (s, 1H), 8.51 (d, J=2.6 Hz, 1H), 8.21 (d, J=8.8 Hz, 1H), 8.11-8.01 (m, 2H), 7.54-7.40 (m, 1H), 7.34-7.23 (m, 1H), 7.17-7.07 (m, 1H), 4.09-3.95 (m, 1H), 3.91 (s, 3H), 1.72-1.44 (m, 2H), 1.19 (d, J=6.6 Hz, 3H), 0.89 (t, J=7.4 Hz, 3H).

### Example 57—Synthesis of 6-[3-(5-Chloro-2-methoxypyridine-3-sulfonamido)-2,6-difluorophenyl]-7-fluoro-N-(pyrrolidin-3-yl)-1H-indazole-3-carboxamide

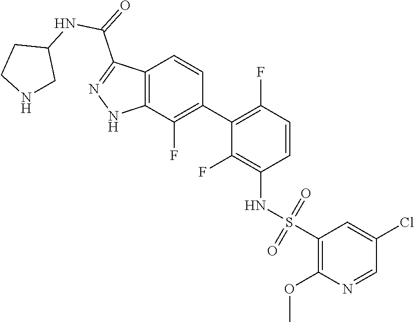 [SMILES: COc1ncc(Cl)cc1S(=O)(=O)Nc1ccc(F)c(-c2ccc3c(C(=O)NC4CCNC4)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (150 mg, 0.3 mmol, 1 equiv), CH₃CN (3.00 mL), tert-butyl 3-aminopyrrolidine-1-carboxylate (109 mg, 0.6 mmol, 2 equiv), NMI (84 mg, 1 mmol, 3.5 equiv), TCFH (123 mg, 0.4 mmol, 1.2 equiv). The resulting solution was stirred overnight at room temperature. The resulting mixture was concentrated under vacuum. The residue was diluted in DCM (2 mL). TFA (2 mL) was added to the mixture. The resulting reaction mixture was stirred for 1 h at room temperature. The resulting mixture was concentrated under vacuum. The residue was diluted in MeOH (4 mL). The filtrate was purified by Prep-HPLC with the following conditions: Column, T3, 19*150 mm, 5 μm; mobile phase, Water (0.1% FA) and CH₃CN (24% CH₃CN up to 40% in 7 min); Detector, 254 nm & 220 nm. 6-[3-(5-Chloro-2-methoxypyridine-3-sulfonamido)-2,6-difluorophenyl]-7-fluoro-N-(pyrrolidin-3-yl)-1H-indazole-3-carboxamide (20 mg, 12% yield) was isolated as a white solid. LCMS (ES, m/z): [M+H]⁺: 581. ¹H NMR (300 MHz, DMSO-d₆) δ 8.83 (d, J=7.2 Hz, 1H), 8.23 (d, J=2.6 Hz, 1H), 8.10-7.90 (m, 2H), 7.33-7.10 (m, 2H), 7.01-6.81 (m, 1H), 4.75-4.55 (m, 1H), 3.81 (s, 3H), 3.43-3.37 (m, 2H), 3.27-3.22 (m, 2H), 2.32-2.16 (m, 1H), 2.12-1.98 (m, 1H).

### Example 58—Synthesis of 6-[3-(5-Chloro-2-methoxypyridine-3-sulfonamido)-2,6-difluorophenyl]-7-fluoro-N-propyl-1H-indazole-3-carboxamide

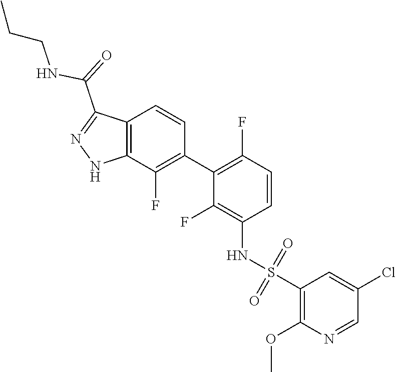 [SMILES: CCCNC(=O)c1n[nH]c2c(F)c(-c3c(F)ccc(NS(=O)(=O)c4cc(Cl)cnc4OC)c3F)ccc12]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), propylamine (23 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, Xselect Fluoro-Phenyl, 19*150 mm, 5 μm; mobile phase, Water (0.1% FA) and CH₃CN (48% CH3CN up to 70% in 7 min); Detector, 254 nm & 220 nm. 6-[3-(5-Chloro-2-methoxypyridine-3-sulfonamido)-2,6-difluorophenyl]-7-fluoro-N-propyl-1H-indazole-3-carboxamide (38 mg, 35% yield) was isolated as a white solid. LCMS-PH-HBC-099-0 (ES, m/z): [M+H]+: 554. ¹H NMR-PH-HBC-099-0 (300 MHz, DMSO-d₆) δ 14.30 (s, 1H), δ 10.43 (s, 1H). 8.67-8.33 (m, 2H), 8.17-7.85 (m, 2H), 7.46 (td, J=8.9, 5.8 Hz, 1H), 7.41-7.17 (m, 1H), 7.13 (dd, J=8.4, 5.8 Hz, 1H), 3.91 (s, 3H), 1.58 (q, J=7.3 Hz, 2H), 0.91 (t, J=7.4 Hz, 3H).

### Example 59—Synthesis of 6-[3-(5-Chloro-2-methoxypyridine-3-sulfonamido)-2,6-difluorophenyl]-7-fluoro-N-(pentan-2-yl)-1H-indazole-3-carboxamide

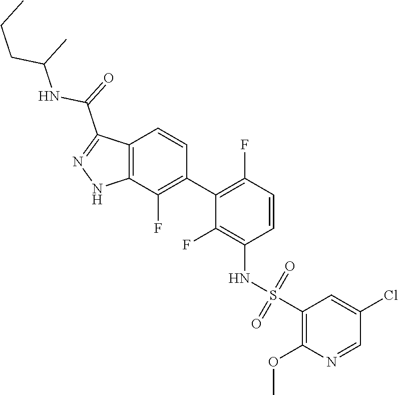 [SMILES: CCCC(C)NC(=O)c1n[nH]c2c(F)c(-c3c(F)ccc(NS(=O)(=O)c4cc(Cl)cnc4OC)c3F)ccc12]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (5 mL), NMI (56 mg, 0.7 mmol, 3.5 equiv), 2-pentanamine (34 mg, 0.4 mmol, 2 equiv) and TCFH (82 mg, 0.3 mmol, 1.5 equiv). The resulting solution was stirred overnight at room temperature, then quenched by the addition of 1 mL of water. The mixture was purified by Prep-HPLC with the following conditions: T3, 19*150 mm, 5 μm; mobile phase, Water (0.1% FA) and CH₃CN (58% CH₃CN up to 78 in 7 min); Detector, 254 nm & 220 nm. 6-[3-(5-Chloro-2-methoxypyridine-3-sulfonamido)-2,6-difluorophenyl]-7-fluoro-N-(pentan-2-yl)-1H-indazole-3-carboxamide (27 mg, 24% yield) was isolated as a white solid. LCMS (ES, m/z):582 [M+H]+. ¹H NMR (300 MHz, DMSO-d₆) δ 8.41 (d, J=2.6 Hz, 1H), 8.27-8.17 (m, 1H), 8.12-7.83 (m, 2H), 7.39 (d, J=6.4 Hz, 1H), 7.17-7.01 (m, 2H), 4.33-3.99 (m, 1H), 3.87 (s, 3H), 1.83-1.26 (m, 4H), 1.19 (d, J=6.6 Hz, 3H), 0.90 (t, J=7.2 Hz, 3H).

### Example 60—Synthesis of 6-[3-(5-chloro-2-methoxypyridine-3-sulfonamido)-2,6-difluorophenyl]-7-fluoro-N-(4-hydroxybutan-2-yl)-1H-indazole-3-carboxamide

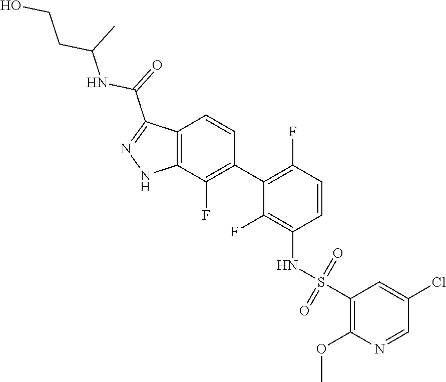 [SMILES: COc1ncc(Cl)cc1S(=O)(=O)Nc1ccc(F)c(-c2ccc3c(C(=O)NC(C)CCO)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH3CN (3 mL), 3-aminobutan-1-ol (35 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, T3, 19*150 mm, 5 μm; mobile phase, Water (0.1% FA) and CH₃CN (38% CH₃CN up to 60% in 7 min); Detector, 254 nm & 220 nm. 6-[3-(5-chloro-2-methoxypyridine-3-sulfonamido)-2,6-difluorophenyl]-7-fluoro-N-(4-hydroxybutan-2-yl)-1H-indazole-3-carboxamide (23 mg, 20% yield) was isolated as a white solid. LCMS (ES, m/z): 584 [M+H]+. ¹H NMR (300 MHz, DMSO-d₆) δ 14.30 (s, 1H), 10.48 (s, 1H), 8.48 (d, J=2.6 Hz, 1H), 8.35 (d, J=8.5 Hz, 1H), 8.14-7.91 (m, 2H), 7.44 (td, J=8.9, 5.8 Hz, 1H), 7.25 (t, J=8.9 Hz, 1H), 7.13 (dd, J=8.3, 5.8 Hz, 1H), 4.50 (t, J=4.7 Hz, 1H), 4.26-3.99 (m, 1H), 3.89 (s, 3H), 3.59-3.41 (m, 2H), 1.73 (ddp, J=19.9, 13.0, 6.2 Hz, 2H), 1.21 (d, J=6.6 Hz, 3H).

### Example 61—Synthesis of 6-[3-(1-Benzofuran-4-sulfonamido)-2,6-difluorophenyl]-7-fluoro-N-methyl-1H-indazole-3-carboxamide

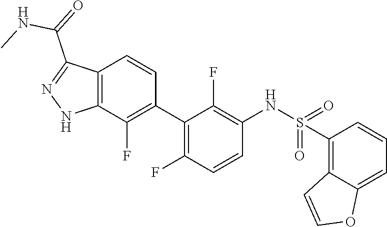 [SMILES: CNC(=O)c1n[nH]c2c(F)c(-c3c(F)ccc(NS(=O)(=O)c4cccc5occc45)c3F)ccc12]

### Part I—Synthesis of 1-Bromo-3-(2,2-dimethoxyethoxy)benzene

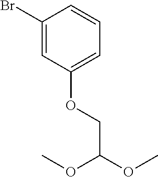

Into a 250-mL 3-necked round-bottom flask, was placed m-bromophenol (5 g, 29 mmol, 1 equiv), DMF (72 ml), K₂CO₃ (4.8 g, 35 mmol, 1.2 equiv) and 2-bromo-1,1-dimethoxyethane (5.4 g, 32 mmol, 1.1 equiv). The resulting solution was stirred for 24 hr at 120 degrees C. The reaction was then cooled and quenched by the addition of 100 mL of water/ice. The resulting solution was extracted with 3×100 mL of ethyl acetate. The organic layers were combined, and washed with 1×100 ml of brine. The mixture was dried over anhydrous sodium sulfate and concentrated under vacuum. The residue was applied onto a silica gel column with ethyl acetate/petroleum ether (1:10). 1-Bromo-3-(2,2-dimethoxyethoxy)benzene (5.0 g, 66% yield) was isolated as a yellow oil.

### Part II—Synthesis of Bromofurans

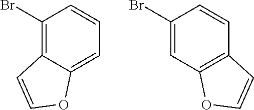

Into a 250-mL 3-necked round-bottom flask, was placed 1-bromo-4-(2,2-dimethoxyethoxy)benzene (5.0 g, 19 mmol, 1 equiv), toluene (100 mL) and H₂PO₄ (0.19 g, 2 mmol, 0.1 equiv). The resulting solution was stirred for 16 hr at 120 degrees C., then cooled and concentrated under vacuum. The residue was applied onto a silica gel column with ethyl acetate/petroleum ether (1:50). The bromofurans were isolated as a mixture (2 g).

### Part III—Synthesis of 4-(benzylsulfanyl)-1-benzofuran and 6-(benzylsulfanyl)-1-benzofuran

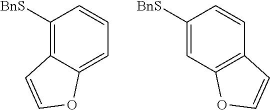

Into a 100-mL 3-necked round-bottom flask purged and maintained with an inert atmosphere of nitrogen, was placed the mixture of 4-bromo-1-benzofuran and 6-bromo-1-benzofuran (2.0 g, 5.1 mmol, 0.5 equiv), dioxane (20 mL), DIEA (2.62 g, 20.3 mmol, 2 equiv), benzyl mercaptan (1.9 g, 15 mmol, 1.5 equiv), xantphos (1.17 g, 2.0 mmol, 0.2 equiv) and Pd₂(dba)₃ (0.93 g, 1 mmol, 0.1 equiv). The resulting solution was stirred for 16 hr at 120 degrees C. in an oil bath. The resulting mixture was concentrated under vacuum. The residue was applied onto a silica gel column with ethyl acetate/petroleum ether (1:50). The mixture of 4-(benzylsulfanyl)-1-benzofuran and 6-(benzylsulfanyl)-1-benzofuran was isolated as a yellow oil (2.5 g).

### Part IV—Synthesis of 1-benzofuran-4-sulfonyl chloride and 1-benzofuran-6-sulfonyl chloride

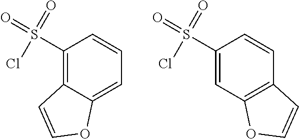 [SMILES: O=S(=O)(Cl)c1cccc2occc12 ; O=S(=O)(Cl)c1ccc2ccoc2c1]

Into a 250-mL 3-necked round-bottom flask, was placed the mixture of 4-(benzylsulfanyl)-1-benzofuran and 6-(benzylsulfanyl)-1-benzofuran (2.5 g, 5.2 mmol, 1 equiv) in CH₃CN (40 mL). This was followed by the addition of 6M HCl (20 mL) dropwise with stirring at 0 degrees C. over 10 min. To this was added NCS (5.56 g, 20.8 mmol, 4 equiv), in portions at 0 degrees C. The resulting solution was stirred for 30 min at 0 degrees C., then diluted with 20 mL of water. The resulting solution was extracted with 3×20 mL of dichloromethane. The extracts were washed with 2×20 of brine, dried over anhydrous sodium sulfate and concentrated under vacuum. This resulted in the mixture of 1-benzofuran-4-sulfonyl chloride and 1-benzofuran-6-sulfonyl chloride as a yellow oil (2.5 g).

### Part V—Synthesis of 6-[3-(1-Benzofuran-4-sulfonamido)-2,6-difluorophenyl]-7-fluoro-N-methyl-1-[[2-(trimethylsilyl)ethoxy]methyl]indazole-3-carboxamide and 6-[3-(1-benzofuran-6-sulfonamido)-2,6-difluorophenyl]-7-fluoro-N-methyl-1-[[2-(trimethylsilyl)ethoxy]methyl]indazole-3-carboxamide

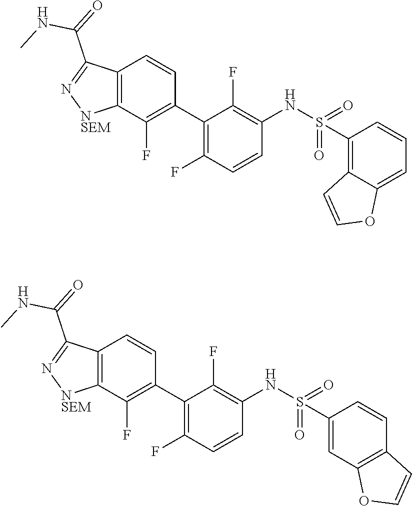 [SMILES: CNC(=O)c1nn(COCC[Si](C)(C)C)c2c(F)c(-c3c(F)ccc(NS(=O)(=O)c4cccc5occc45)c3F)ccc12 ; CNC(=O)c1nn(COCC[Si](C)(C)C)c2c(F)c(-c3c(F)ccc(NS(=O)(=O)c4ccc5ccoc5c4)c3F)ccc12]

Into a 50-mL 3-necked round-bottom flask, was placed 6-(3-amino-2,6-difluorophenyl)-7-fluoro-N-methyl-1-[[2-(trimethylsilyl)ethoxy]methyl]indazole-3-carboxamide (1.0 g, 2.2 mmol, 1 equiv), DCM (20 mL) and pyridine (0.88 g, 11 mmol, 5 equiv). This was followed by addition of the mixture of 1-benzofuran-6-sulfonyl chloride and 1-benzofuran-4-sulfonyl chloride (1.5 g, 3.3 mmol, 1.5 equiv) in DCM (1 mL) dropwise with stirring at 0 degrees C. The resulting solution was stirred for 30 min at room temperature, then concentrated under vacuum. The residue was purified by Prep-HPLC with the following conditions: Water (0.1% FA) and ACN (71.0% ACN up to 86.0% in 7 min, hold 95.0% in 1 min, down to 71.0% in 1 min. 200 mg (14.29%) 6-[3-(1-Benzofuran-4-sulfonamido)-2,6-difluorophenyl]-7-fluoro-N-methyl-1-[[2-(trimethylsilyl)ethoxy]methyl]indazole-3-carboxamide (200 mg, 14% yield) and 6-[3-(1-benzofuran-6-sulfonamido)-2,6-difluorophenyl]-7-fluoro-N-methyl-1-[[2-(trimethylsilyl)ethoxy]methyl]indazole-3-carboxamide (200 mg, 14% yield) were isolated as white solids. LCMS (ES, m/z): [M+H]⁺: 631.

### Part VI—Synthesis of 6-[3-(1-Benzofuran-4-sulfonamido)-2,6-difluorophenyl]-7-fluoro-N-methyl-1H-indazole-3-carboxamide

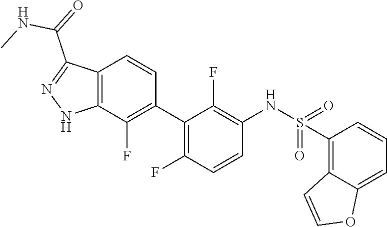 [SMILES: CNC(=O)c1n[nH]c2c(F)c(-c3c(F)ccc(NS(=O)(=O)c4cccc5occc45)c3F)ccc12]

Into a 50-mL round-bottom flask, was placed 6-[3-(1-benzofuran-4-sulfonamido)-2,6-difluorophenyl]-7-fluoro-N-methyl-1-[[2-(trimethylsilyl)ethoxy]methyl]indazole-3-carboxamide (100 mg, 0.16 mmol, 1 equiv) and DCM (5 mL). This was followed by addition of TFA (1 mL) dropwise with stirring at 0 degrees C. The resulting solution was stirred for 6 hr at room temperature, then diluted with 10 mL of water. The pH was adjusted to 8 with NaHCO₃ (2 mol/L). The resulting solution was extracted with 3×10 mL of DCM, and the organics combined, dried over anhydrous sodium sulfate and concentrated under vacuum. The crude product (120 mg) was purified by Prep-HPLC with the following conditions, Water (20 MMOL/L NH₄HCO₃) and ACN (10% Phase B up to 65% in 20 min); 6-[3-(1-Benzofuran-4-sulfonamido)-2,6-difluorophenyl]-7-fluoro-N-methyl-1H-indazole-3-carboxamide (31.6 mg, 40% yield) was isolated as a white solid. LCMS (ES, m/z): [M+H]⁺: 501. ¹H NMR (300 MHz, DMSO-d₆) δ 14.26 (s, 1H), 8.49 (d, J=4.8 Hz, 1H), 8.14 (d, J=2.2 Hz, 1H), 8.00 (d, J=8.3 Hz, 1H), 7.88 (d, J=8.2 Hz, 1H), 7.66 (dd, J=7.7, 0.9 Hz, 1H), 7.46 (t, J=8.0 Hz, 1H), 7.36 (td, J=9.0, 5.9 Hz, 1H), 7.16 (t, J=9.0 Hz, 1H), 7.03 (dd, J=2.3, 0.9 Hz, 1H), 6.97 (dd, J=8.4, 5.8 Hz, 1H), 2.83 (d, J=4.7 Hz, 3H).

### Example 62—Synthesis of 6-[3-(1H-1,3-Benzodiazole-4-sulfonamido)-2,6-difluorophenyl]-7-fluoro-N-methyl-1H-indazole-3-carboxamide

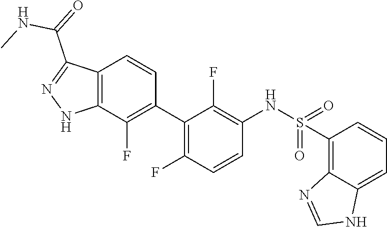 [SMILES: CNC(=O)c1n[nH]c2c(F)c(-c3c(F)ccc(NS(=O)(=O)c4cccc5[nH]cnc45)c3F)ccc12]

### Part I—Synthesis of 3-(Benzylsulfanyl)-2-nitroaniline

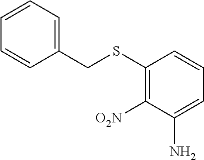 [SMILES: Nc1cccc(SCc2ccccc2)c1[N+](=O)[O-]]

Into a 250-mL round-bottom flask, was placed 3-fluoro-2-nitroaniline (3 g, 19 mmol, 1 equiv), DMF (30 mL) and K₂CO₃ (8.0 g, 58 mmol, 3 equiv). This was followed by the addition of benzyl mercaptan (3.6 g, 29 mmol, 1.5 equiv) dropwise with stirring at 30 degrees C. The resulting solution was stirred overnight at 30 degrees C. The reaction was then quenched by the addition of 50 mL of water. The resulting solution was extracted with 2×50 mL of ethyl acetate. The organics were washed with 3×50 ml of water. The mixture was dried over anhydrous sodium sulfate and concentrated. The residue was applied onto a silica gel column, eluting with ethyl acetate/PE (1/3). The collected fractions were combined and concentrated. 3-(Benzylsulfanyl)-2-nitroaniline (4.6 g, 87% yield) was obtained as a yellow solid. LCMS (ES, m/z): [M+H]⁺: 261.

### Part II—Synthesis of 3-(benzylsulfanyl)benzene-1,2-diamine

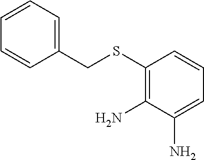 [SMILES: Nc1cccc(SCc2ccccc2)c1N]

Into a 250-mL round-bottom flask, was placed 3-(benzylsulfanyl)-2-nitroaniline (4.6 g, 17.7 mmol, 1 equiv), MeOH (90 mL), Zn (5.78 g, 0.09 mol, 5 equiv) and NH₄Cl (4.73 g, 88 mmol, 5 equiv). The resulting solution was stirred for 3 hr at 25 degrees C. The solids were removed by filtration and the filtrate concentrated. The residue was suspended in 100 mL of water and extracted with 3×100 mL of ethyl acetate. The organics were dried over anhydrous sodium sulfate and concentrated to give 3-(benzylsulfanyl)benzene-1,2-diamine (4 g, 98% yield) as a brown solid. LCMS (ES, m/z): [M+H]⁺: 231.

### Part III—Synthesis of 4-(benzylsulfanyl)-1H-1,3-benzodiazole

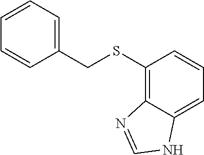 [SMILES: c1ccc(CSc2cccc3[nH]cnc23)cc1]

Into a 250-mL round-bottom flask, was placed 3-(benzylsulfanyl)benzene-1,2-diamine (4.95 g, 21.5 mmol, 1 equiv) and formic acid (100 mL). The resulting solution was stirred for 2 hr at 100 degrees C. The reaction mixture was cooled to room temperature and concentrated. The residue was applied onto a silica gel column, eluting with ethyl acetate/PE (2/1). The collected fractions were combined and concentrated to give 4-(benzylsulfanyl)-1H-1,3-benzodiazole (4.6 g, 89% yield) as a yellow solid. LCMS (ES, m/z): [M+H]⁺: 241.

### Part IV—Synthesis of 4-(benzylsulfanyl)-1-[[2-(trimethylsilyl)ethoxy]methyl]-1,3-benzodiazole

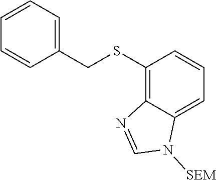 [SMILES: C[Si](C)(C)CCOCn1cnc2c(SCc3ccccc3)cccc21]

Into a 100-mL 3-necked round-bottom flask, was placed 4-(benzylsulfanyl)-1H-1,3-benzodiazole (840 mg, 3.5 mmol, 1 equiv) in THE (17 mL). This was followed by the addition of NaH (210 mg, 5.2 mmol, 1.5 equiv, 60%) in portions at 0 degrees C. To this was added SEM-Cl (699 mg, 4.2 mmol, 1.2 equiv) dropwise at 0 degrees C. The resulting solution was stirred for 1 hr at 0 degrees C., then quenched by the addition of 30 mL of water/ice. The resulting solution was extracted with 3×30 mL of ethyl acetate and the organic layers combined and dried over anhydrous sodium sulfate, then concentrated. The residue was applied onto a silica gel column eluting with ethyl acetate/PE (1/2). The collected fractions were combined and concentrated to give 4-(benzylsulfanyl)-1-[[2-(trimethylsilyl)ethoxy]methyl]-1,3-benzodiazole (1.0 g, 77% yield) as a brown solid. LCMS (ES, m/z): [M+H]⁺: 371.

### Part V—Synthesis of 1-[[2-(trimethylsilyl)ethoxy]methyl]-1,3-benzodiazole-4-sulfonyl chloride

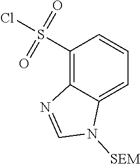 [SMILES: C[Si](C)(C)CCOCn1cnc2c(S(=O)(=O)Cl)cccc21]

Into a 50-mL round-bottom flask, was placed MeCN (10 mL) and 2N HCl (2 mL), then NCS (721 mg, 5.4 mmol, 4 equiv), in portions at 0° C. To this was added 4-(benzylsulfanyl)-1-[[2-(trimethylsilyl)ethoxy]methyl]-1,3-benzodiazole (500 mg, 1.3 mmol, 1 equiv) at 0° C. The resulting solution was stirred for 1 hr in a water/ice bath. The reaction was quenched by the addition of 20 mL of water and the resulting solution was extracted with 2×20 mL of dichloromethane. The combined extracts were dried over anhydrous sodium sulfate and concentrated to give 1-[[2-(trimethylsilyl)ethoxy]methyl]-1,3-benzodiazole-4-sulfonyl chloride (800 mg, 85% yield) as a yellow oil.

### Part VI—Synthesis of 6-[2,6-Difluoro-3-(1-[[2-(trimethylsilyl)ethoxy]methyl]-1,3-benzodiazole-4-sulfonamido) phenyl]-7-fluoro-N-methyl-1-[[2-(trimethylsilyl)ethoxy]methyl]indazole-3-carboxamide

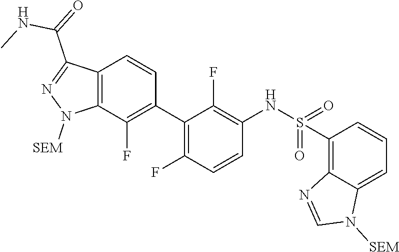 [SMILES: CNC(=O)c1nn(COCC[Si](C)(C)C)c2c(F)c(-c3c(F)ccc(NS(=O)(=O)c4cccc5c4ncn5COCC[Si](C)(C)C)c3F)ccc12]

Into a 40-mL vial, was placed 6-(3-amino-2,6-difluorophenyl)-7-fluoro-N-methyl-1-[[2-(trimethylsilyl)ethoxy]methyl] indazole-3-carboxamide (300 mg, 0.7 mmol, 1 equiv), DCM (10 mL), pyridine (1.05 g, 13.3 mmol, 20 equiv) and then 1-[[2-(trimethylsilyl)ethoxy]methyl]-1,3-benzodiazole-4-sulfonyl chloride (693 mg, 2 mmol, 3 equiv). The resulting solution was stirred for 1 hr at 25 degrees C., then concentrated. The crude product was purified by Flash-Prep-HPLC with the following conditions (IntelFlash-1): Column, C18; mobile phase, 0.1% NH₃.H₂O and MeCN, from 5% increasing to 70% within 20 min; Detector, 220 nm. 6-[2,6-Difluoro-3-(1-[[2-(trimethylsilyl)ethoxy]methyl]-1,3-benzodiazole-4-sulfonamido) phenyl]-7-fluoro-N-methyl-1-[[2-(trimethylsilyl)ethoxy]methyl]indazole-3-carboxamide (360 mg, 71% yield) was isolated as a yellow solid. LCMS (ES, m/z): [M+H]⁺: 761.

### Part VII—Synthesis of 6-[3-(1H-1,3-Benzodiazole-4-sulfonamido)-2,6-difluorophenyl]-7-fluoro-N-methyl-1H-indazole-3-carboxamide

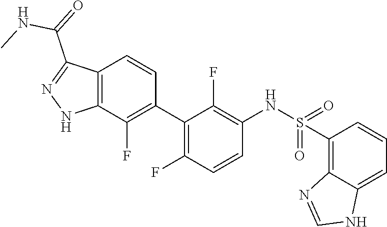 [SMILES: CNC(=O)c1n[nH]c2c(F)c(-c3c(F)ccc(NS(=O)(=O)c4cccc5[nH]cnc45)c3F)ccc12]

Into a 50-mL round-bottom flask, was placed 6-[2,6-difluoro-3-(1-[[2-(trimethylsilyl)ethoxy] methyl]-1,3-benzodiazole-4-sulfonamido)phenyl]-7-fluoro-N-methyl-1-[[2-(trimethylsilyl)ethoxy]methyl]indazole-3-carboxamide (180 mg, 0.24 mmol, 1 equiv), DCM (5 mL) and TFA (2 mL). The resulting solution was stirred for 2 hr at 25 degrees C., then concentrated. The residue was dissolved in 2 mL of MeOH and the pH adjusted to 8 with 7M NH₃ in MeOH. The crude product was purified by Prep-HPLC with the following conditions: Column, XBridge Prep C18 OBD Column, 5 μm, 19*150 mm; mobile phase, Water (0.05% NH₃/H₂O) and ACN (10% PhaseB up to 40% in 11 min); Detector, 220 nm. 6-[3-(1H-1,3-Benzodiazole-4-sulfonamido)-2,6-difluorophenyl]-7-fluoro-N-methyl-1H-indazole-3-carboxamide (24 mg, 20% yield) was isolated as an off-white solid. LCMS (ES, m/z): [M+H]⁺: 501. ¹H NMR (300 MHz, DMSO-d₆) δ 14.24 (s, 1H), 8.49 (q, J=4.7 Hz, 1H), 8.21 (d, J=13.5 Hz, 1H), 8.00 (d, J=8.4 Hz, 1H), 7.81 (d, J=7.9 Hz, 1H), 7.60-7.52 (m, 1H), 7.28 (td, J=8.5, 7.7, 5.3 Hz, 2H), 7.11-6.92 (m, 2H), 2.87-2.80 (m, 3H).

### Example 63—Synthesis of 6-[2,6-Difluoro-3-(3-methyl-1,3-benzodiazole-4-sulfonamido)phenyl]-7-fluoro-N-methyl-1H-indazole-3-carboxamide

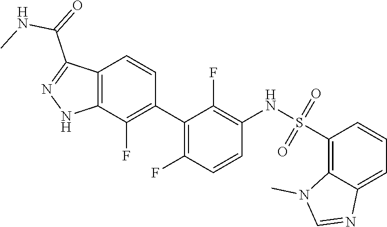 [SMILES: CNC(=O)c1n[nH]c2c(F)c(-c3c(F)ccc(NS(=O)(=O)c4cccc5ncn(C)c45)c3F)ccc12]

### Part I—Synthesis of 4-(benzylsulfanyl)-1-methyl-1,3-benzodiazole (1.7 g, 45% yield) and 7-(benzylsulfanyl)-1-methyl-1,3-benzodiazole

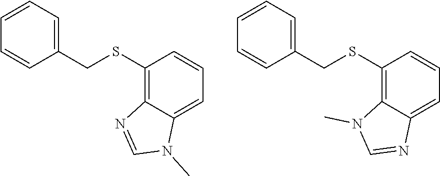

Into a 250-mL 3-necked round-bottom flask, was placed 4-(benzylsulfanyl)-1H-1,3-benzodiazole (3.6 g, 15 mmol, 1 equiv), THE (75 mL) and Cs₂CO₃ (14.6 g, 45 mmol, 3 equiv). This was followed by the addition of CH₃I (6.4 g, 45 mmol, 3 equiv) dropwise with stirring at 0 degrees C. The resulting solution was stirred overnight at 25 degrees C. The solids were removed by filtration and the filtrate concentrated. The crude product was purified by Prep-HPLC with the following conditions: Column, WelFlash TM C18-I, Spherical C18 20-40 m, 330 g; mobile phase, Water (20 mmoL/L NH₄HCO₃) and ACN (30% Phase B up to 50% in 10 min); Detector, uv 220 nm. This gave in 4-(benzylsulfanyl)-1-methyl-1,3-benzodiazole (1.7 g, 45% yield) and 7-(benzylsulfanyl)-1-methyl-1,3-benzodiazole (500 mg, 13% yield) as yellow solids. LCMS (ES, m/z): [M+H]⁺: 255.

### Part II—Synthesis of 3-methyl-1,3-benzodiazole-4-sulfonyl chloride

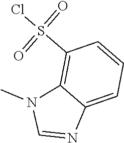

Into a 50-mL round-bottom flask, was placed MeCN (6 mL) and 6 M HCl (1.2 mL). This was followed by the addition of NCS (840 mg, 6.3 mmol, 4 equiv), in portions at 0 degrees C. To this was added 7-(benzylsulfanyl)-1-methyl-1,3-benzodiazole (400 mg, 1.6 mmol, 1 equiv) at 0 degrees C. The resulting solution was stirred for 1 h in a water/ice bath. The reaction was then quenched by the addition of 20 mL of water. The resulting solution was extracted with 2×20 mL of dichloromethane and the extracts were dried over anhydrous sodium sulfate and concentrated. The residue was applied onto a silica gel column eluting with ethyl acetate/petroleum ether (1:1). The collected fractions were combined and concentrated to give 3-methyl-1,3-benzodiazole-4-sulfonyl chloride (360 mg, 99% yield) as a yellow oil.

### Part III—Synthesis of 6-[2,6-Difluoro-3-(3-methyl-1,3-benzodiazole-4-sulfonamido)phenyl]-7-fluoro-N-methyl-1-[[2-(trimethylsilyl)ethoxy]methyl]indazole-3-carboxamide

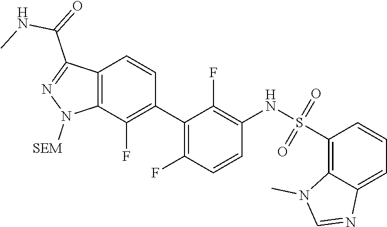 [SMILES: CNC(=O)c1nn(COCC[Si](C)(C)C)c2c(F)c(-c3c(F)ccc(NS(=O)(=O)c4cccc5ncn(C)c45)c3F)ccc12]

Into a 40-mL vial, was placed 6-(3-amino-2,6-difluorophenyl)-7-fluoro-N-methyl-1-[[2-(trimethylsilyl)ethoxy] methyl]indazole-3-carboxamide (200 mg, 0.4 mmol, 1 equiv), DCM (4 mL), pyridine (351 mg, 4.4 mmol, 10 equiv) and 3-methyl-1,3-benzodiazole-4-sulfonyl chloride (154 mg, 0.7 mmol, 1.5 equiv). The resulting solution was stirred overnight at 35 degrees C. The reaction mixture was cooled and concentrated. The crude product was purified by Flash-Prep-HPLC with the following conditions (IntelFlash-1): Column, WelFlash TM C18-I, Spherical C18 20-40 m, 120 g; mobile phase, 0.1% FA and MeCN, from 5% increasing to 60% in 20 min; Detector, 220 nm. 6-[2,6-Difluoro-3-(3-methyl-1,3-benzodiazole-4-sulfonamido)phenyl]-7-fluoro-N-methyl-1-[[2-(trimethylsilyl)ethoxy]methyl]indazole-3-carboxamide (250 mg, 87% yield) was isolated as a yellow solid. LCMS (ES, m/z): [M+H]⁺: 645.

### Part IV—Synthesis of 6-[2,6-Difluoro-3-(3-methyl-1,3-benzodiazole-4-sulfonamido)phenyl]-7-fluoro-N-methyl-1H-indazole-3-carboxamide

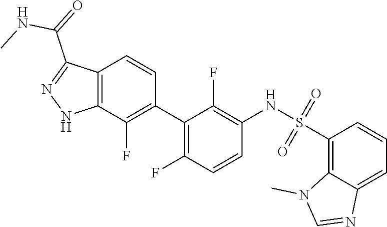 [SMILES: CNC(=O)c1n[nH]c2c(F)c(-c3c(F)ccc(NS(=O)(=O)c4cccc5ncn(C)c45)c3F)ccc12]

Into a 50-mL round-bottom flask, was placed 6-[2,6-difluoro-3-(3-methyl-1,3-benzodiazole-4-sulfonamido)phenyl]-7-fluoro-N-methyl-1-[[2-(trimethylsilyl)ethoxy]methyl] indazole-3-carboxamide (250 mg, 0.4 mmol, 1 equiv), DCM (5 mL) and then TFA (2 mL). The resulting solution was stirred for 2 h, then concentrated. The resulting solution was diluted with 3 mL of MeOH. The pH was adjusted to 8 with NH₃ (7 M in MeOH) and the mixture concentrated. The crude product was purified by Flash-Prep-HPLC with the following conditions: Column, welch Vltimate XB-C18, 50×250 mm, 10 μm, mobile phase, 0.1% FA and MeCN=5% increasing from 1% to 70% in 20 min. 6-[2,6-Difluoro-3-(3-methyl-1,3-benzodiazole-4-sulfonamido)phenyl]-7-fluoro-N-methyl-1H-indazole-3-carboxamide (102 mg, 51% yield) was isolated as an off-white solid. LCMS (ES, m/z): [M+H]⁺: 515. ¹H NMR (300 MHz, DMSO-d₆) δ 14.31 (s, 1H), 10.60 (s, 1H), 8.52 (d, J=5.0 Hz, 1H), 8.36 (s, 1H), 8.08-7.95 (m, 2H), 7.72 (dd, J=7.8, 1.1 Hz, 1H), 7.45-7.29 (m, 2H), 7.24 (td, J=9.0, 1.5 Hz, 1H), 7.13-7.02 (m, 1H), 4.19 (s, 3H), 2.84 (d, J=4.7 Hz, 3H).

### Example 64—Synthesis of 5-chloro-N-[2,4-difluoro-3-[7-fluoro-3-(1,3-oxazol-2-yl)-1H-indazol-6-yl]phenyl]-2-methoxypyridine-3-sulfonamide

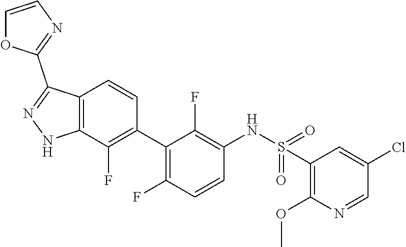 [SMILES: COc1ncc(Cl)cc1S(=O)(=O)Nc1ccc(F)c(-c2ccc3c(-c4ncco4)n[nH]c3c2F)c1F]

### Part I—Synthesis of 6-bromo-7-fluoro-3-(1,3-oxazol-2-yl)-1-[[2-(trimethylsilyl)ethoxy]methyl] indazole

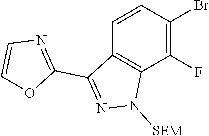 [SMILES: C[Si](C)(C)CCOCn1nc(-c2ncco2)c2ccc(Br)c(F)c21]

To a stirred solution of 2-(tetrabutylstannyl)-1,3-oxazole (1.0 g, 2.8 mmol, 1 equiv) in dioxane (20 mL) were added 6-bromo-7-fluoro-3-iodo-1-[[2-(trimethylsilyl)ethoxy]methyl]indazole (See Example 23) (1.97 g, 4.2 mmol, 1.5 equiv), tris(furan-2-yl)phosphane (129 mg, 560 μmol, 0.2 equiv) and Pd(PPh₃)₂Cl₂ (196 mg, 280 μmol, 0.1 equiv). The resulting solution was stirred at 100° C. under nitrogen for 16 hours. After cooling to room temperature, water (200 mL) was added and the mixture was extracted with ethyl acetate (3×200 mL). The organics were washed with brine (2×50 mL), dried over anhydrous Na₂SO₄ and concentrated to give a crude product which was purified by column chromatography over silica gel (eluent: PE:EA=10:1) to afford 6-bromo-7-fluoro-3-(1,3-oxazol-2-yl)-1-[[2-(trimethylsilyl)ethoxy]methyl]indazole (700 mg, 61% yield) as a yellow solid. LCMS (ES, m/z): [M+H]⁺: 412.

### Part II—Synthesis of 7-fluoro-3-(1,3-oxazol-2-yl)-6-(4,4,5,5-tetramethyl-1,3,2-dioxaborolan-2-yl)-1-[[2-(trimethylsilyl)ethoxy]methyl]indazole

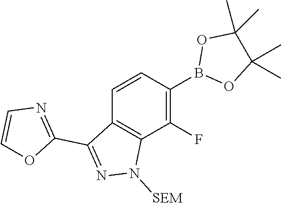 [SMILES: CC1(C)OB(c2ccc3c(-c4ncco4)nn(COCC[Si](C)(C)C)c3c2F)OC1(C)C]

To a solution of 6-bromo-7-fluoro-3-(1,3-oxazol-2-yl)-1-[[2-(trimethylsilyl)ethoxy]methyl] indazole (700 mg, 1.7 mmol, 1 eq) in dioxane (10 mL) were added bis(pinacolato)diboron (1.07 g, 4.2 mmol, 2.5 eq), KOAc (416 mg, 4.2 mmol, 2.5 eq), Pd(dppf)Cl₂ (248 mg, 0.34 mmol, 0.2 eq). The resulting solution was stirred at 100° C. under nitrogen for 16 hours. After cooling to room temperature, water (100 mL) was added and the mixture was extracted with ethyl acetate (3×50 mL). The combined organics were washed with brine (2×20 mL), dried over anhydrous Na₂SO₄, filtered and concentrated to give crude product, which was purified by column chromatography over silica gel (eluent: PE:EA=3:1) to afford 7-fluoro-3-(1,3-oxazol-2-yl)-6-(4,4,5,5-tetramethyl-1,3,2-dioxaborolan-2-yl)-1-[[2-(trimethylsilyl)ethoxy]methyl]indazole (700 mg, 90% yield) as a yellow solid. LCMS (ES, m/z): [M+H]⁺: 333.

### Part III—Synthesis of N-(3-bromo-2,4-difluorophenyl)-5-chloro-2-methoxypyridine-3-sulfonamide

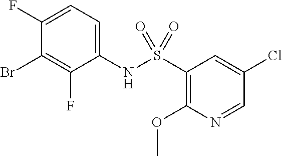 [SMILES: COc1ncc(Cl)cc1S(=O)(=O)Nc1ccc(F)c(Br)c1F]

To a solution of 3-bromo-2,4-difluoroaniline (5 g, 24.03 mmol, 1.0 eq) in DCM (100 mL) were added 5-chloro-2-methoxypyridine-3-sulfonyl chloride (8.73 g, 36.06 mmol, 1.5 eq) and pyridine (5.7 g, 72.11 mmol, 3.0 eq). The resulting solution was stirred for 1 hour at room temperature. The reaction was concentrated and purified by column chromatography over silica gel (eluent: PE:EA=8:1) to afford the desired product as a white solid (7 g, yield 70%). LCMS (ES, m/z): [M+H]+: 412. ¹H NMR (300 MHz, Chloroform-d) δ 8.30 (d, J=2.6 Hz, 1H), 8.05 (d, J=2.6 Hz, 1H), 7.56 (td, J=8.9, 5.4 Hz, 1H), 7.28 (d, J=3.4 Hz, 1H), 6.96 (ddd, J=9.4, 7.6, 2.1 Hz, 1H), 4.16 (s, 3H).

### Part IV—Synthesis of 5-chloro-N-[2,4-difluoro-3-[7-fluoro-3-(1,3-oxazol-2-yl)-1-[[2-(trimethylsilyl)ethoxy]methyl]indazol-6-yl]phenyl]-2-methoxypyridine-3-sulfonamide

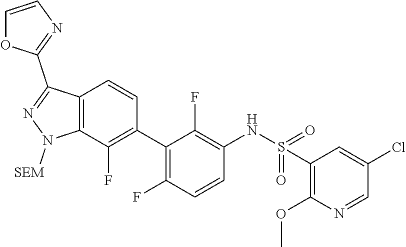 [SMILES: COc1ncc(Cl)cc1S(=O)(=O)Nc1ccc(F)c(-c2ccc3c(-c4ncco4)nn(COCC[Si](C)(C)C)c3c2F)c1F]

To a stirred solution of 7-fluoro-3-(1,3-oxazol-2-yl)-6-(4,4,5,5-tetramethyl-1,3,2-dioxaborolan-2-yl)-1-[[2-(trimethylsilyl)ethoxy]methyl]indazole (450 mg, 0.98 mmol, 1 equiv) in dioxane (10 mL) and H₂O (2 mL) were added N-(3-bromo-2,4-difluorophenyl)-5-chloro-2-methoxypyridine-3-sulfonamide (486 mg, 1.2 mmol, 1.2 equiv), K₂CO₃ (336 mg, 2.4 mmol, 2 eq) and Pd(dppf)Cl₂ (72 mg, 0.1 mmol, 0.1 equiv). The reaction mixture was stirred at 100° C. for 16 hours under nitrogen atmosphere. Once cooled, water (100 mL) was added, and the mixture was extracted with EA (3×50 mL). The combined organics were washed with brine (2×20 mL), dried over anhydrous Na₂SO₄, filtered and concentrated in vacuum. The residue was purified by column chromatograph over silica gel (PE:EA=2:1) to afford the 5-chloro-N-[2,4-difluoro-3-[7-fluoro-3-(1,3-oxazol-2-yl)-1-[[2-(trimethylsilyl)ethoxy]methyl]indazol-6-yl]phenyl]-2-methoxypyridine-3-sulfonamide (350 mg, 53%) as a white solid. LCMS (ES, m/z): [M+H]⁺: 666.

### Part V—Synthesis of 5-chloro-N-[2,4-difluoro-3-[7-fluoro-3-(1,3-oxazol-2-yl)-1H-indazol-6-yl]phenyl]-2-methoxypyridine-3-sulfonamide

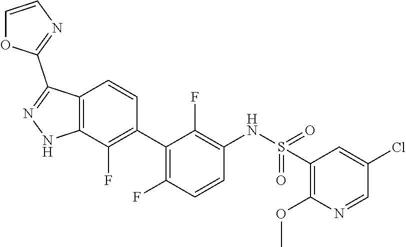 [SMILES: COc1ncc(Cl)cc1S(=O)(=O)Nc1ccc(F)c(-c2ccc3c(-c4ncco4)n[nH]c3c2F)c1F]

To a stirred solution of 5-chloro-N-[2,4-difluoro-3-[7-fluoro-3-(1,3-oxazol-2-yl)-1-[[2-(trimethylsilyl)ethoxy]methyl]indazol-6-yl]phenyl]-2-methoxypyridine-3-sulfonamide (150 mg, 0.23 mmol, 1 equiv) in DCM (8 mL) was added TFA (2 mL). The reaction mixture was stirred at room temperature for 1 hour and then concentrated. The residue was purified by prep-HPLC with the following conditions: Column, welch XB-C18, 50×250 mm, 10 μm, mobile phase, Mobile Phase A: 0.1% FA in Water, Mobile Phase B: CAN (30% up to 60% in 15 min) to afford 5-chloro-N-[2,4-difluoro-3-[7-fluoro-3-(1,3-oxazol-2-yl)-1H-indazol-6-yl]phenyl]-2-methoxypyridine-3-sulfonamide (40 mg, 33%) as a white solid. LCMS (ES, m/z): [M+H]⁺: 536. ¹H NMR (300 MHz, DMSO-d₆) δ 14.48 (s, 1H), 10.47 (s, 1H), 8.51 (d, J=2.6 Hz, 1H), 8.33 (s, 1H), 8.19-8.05 (m, 2H), 7.53 (d, J=0.8 Hz, 1H), 7.47 (td, J=8.9, 5.9 Hz, 1H), 7.34-7.26 (m, 1H), 7.21 (dd, J=8.3, 5.9 Hz, 1H), 3.91 (s, 3H).

### Example 65—Synthesis of 6-[2,6-difluoro-3-(5-fluoro-2-methylpyridine-3-sulfonamido)phenyl]-7-fluoro-N-methyl-1H-indazole-3-carboxamide

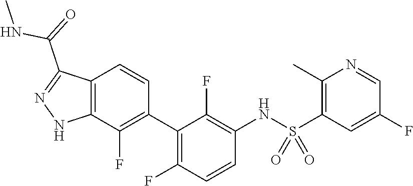 [SMILES: CNC(=O)c1n[nH]c2c(F)c(-c3c(F)ccc(NS(=O)(=O)c4cc(F)cnc4C)c3F)ccc12]

### Part I—Synthesis of 3-bromo-5-fluoro-2-methylpyridine

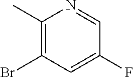

To a stirred mixture of 2,3-dibromo-5-fluoropyridine (9.0 g, 35.3 mmol, 1 equiv), methylboronic acid (8.45 g, 141 mmol, 4 equiv) and Na₂CO₃ (7.5 g, 71 mmol, 2 equiv) in dioxane (100 mL) were added H₂O (25 mL) and Pd(dppf)Cl₂ (2.58 g, 3.5 mmol, 0.1 equiv) at room temperature under N₂ atmosphere. The resulting mixture was stirred overnight at 100 degrees C. under N₂ atmosphere. The mixture was allowed to cool, then the solids removed by filtration. The filtrate was concentrated under reduced pressure. The residue was purified by silica gel column chromatography, eluting with PE:EA (98:2) to afford 3-bromo-5-fluoro-2-methylpyridine (1.2 g, 18% yield) as a white solid. LCMS (ES, m/z): [M+H]⁺: 190.

### Part II—Synthesis of 3-(benzylsulfanyl)-5-fluoro-2-methylpyridine

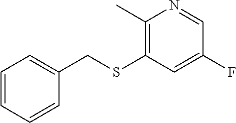 [SMILES: Cc1ncc(F)cc1SCc1ccccc1]

To a stirred mixture of 3-bromo-5-fluoro-2-methylpyridine (1.2 g, 6.3 mmol, 1 equiv), benzyl mercaptan (1.2 g, 9.5 mmol, 1.5 equiv) and DIEA (1.63 g, 12.6 mmol, 2 equiv) in toluene (20 mL) was added XantPhos (0.73 g, 1.3 mmol, 0.2 equiv) and Pd₂(dba)₃. CHCl₃ (0.65 g, 0.63 mmol, 0.1 equiv) at room temperature under N₂ atmosphere. The mixture was stirred for 4 h at 115 degrees C. The reaction was allowed to cool to room temperature and was filtered. The filtrate was concentrated under reduced pressure. The crude product was purified by Column, WelFlash TM C18-I, Spherical C18 20-40 m, 120 g; mobile phase, 0.1% FA and MeCN, from 15% increasing to 60% in 12 min; Detector, 220 nm. It afforded 3-(benzylsulfanyl)-5-fluoro-2-methylpyridine (1 g, 68%) as a yellow solid. LCMS (ES, m/z): [M+H]⁺: 234.

### Part III—Synthesis of 5-fluoro-2-methylpyridine-3-sulfonyl chloride

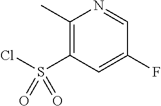

To a stirred solution of 3-(benzylsulfanyl)-5-fluoro-2-methylpyridine (500 mg, 2.1 mmol, 1 equiv) and H₂O (3 mL) in HOAc (10 mL) was added NCS (1.14 g, 8.5 mmol, 4 equiv) in portions below 10 degrees C. The resulting solution was stirred for 2 h at room temperature. The mixture was quenched with water (100 mL). The resulting mixture was extracted with DCM (3×50 mL). The combined organic layers were washed with saturated aqueous NaHCO₃ (1×20 mL), brine (1×20 mL) and dried over anhydrous Na₂SO₄. After filtration, the filtrate was concentrated under reduced pressure to give 5-fluoro-2-methylpyridine-3-sulfonyl chloride (990 mg, crude) as a semi-solid which was used in the next step directly without further purification.

### Part IV—Synthesis of 6-[2,6-difluoro-3-(5-fluoro-2-methylpyridine-3-sulfonamido)phenyl]-7-fluoro-N-methyl-1-[[2-(trimethylsilyl)ethoxy]methyl]indazole-3-carboxamide

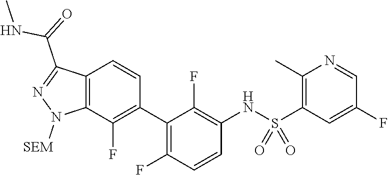 [SMILES: CNC(=O)c1nn(COCC[Si](C)(C)C)c2c(F)c(-c3c(F)ccc(NS(=O)(=O)c4cc(F)cnc4C)c3F)ccc12]

To a stirred solution of 6-(3-amino-2,6-difluorophenyl)-7-fluoro-N-methyl-1-[[2-(trimethylsilyl)ethoxy]methyl]indazole-3-carboxamide (60 mg, 0.1 mmol, 1 equiv) and pyridine (63 mg, 0.8 mmol, 6 equiv) in DCM (10 mL) was added a solution of 5-fluoro-2-methylpyridine-3-sulfonyl chloride (84 mg, 0.4 mmol, 3 equiv) in DCM (2 mL) dropwise at 0 degrees C. The resulting solution was stirred for 2 h at room temperature. The reaction mixture was quenched with water (10 mL). The resulting solution was extracted with DCM (3×20 mL). The combined organics were washed with brine (2×20 mL), dried over anhydrous Na₂SO₄. After filtration, the filtrate was concentrated under reduce pressure. The crude product was purified by Flash-Prep-HPLC with the following conditions: Column, WelFlash TM C18-I, Spherical C18 20-40 m, 120 g; mobile phase, 0.1% FA and MeCN, from 5% increasing to 60% in 12 min; Detector, 220 nm. It gave 6-[2,6-difluoro-3-(5-fluoro-2-methylpyridine-3-sulfonamido)phenyl]-7-fluoro-N-methyl-1-[[2-(trimethylsilyl)ethoxy]methyl]indazole-3-carboxamide (186 mg, 22.4%) as a yellow solid. LCMS (ES, m/z): [M+H]⁺: 624.

### Part V—Synthesis of 6-[2,6-difluoro-3-(5-fluoro-2-methylpyridine-3-sulfonamido)phenyl]-7-fluoro-N-methyl-1H-indazole-3-carboxamide

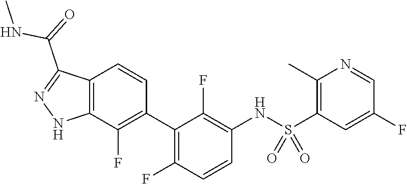 [SMILES: CNC(=O)c1n[nH]c2c(F)c(-c3c(F)ccc(NS(=O)(=O)c4cc(F)cnc4C)c3F)ccc12]

A mixture of 6-[2,6-difluoro-3-(5-fluoro-2-methylpyridine-3-sulfonamido)phenyl]-7-fluoro-N-methyl-1-[[2-(trimethylsilyl)ethoxy]methyl]indazole-3-carboxamide (180 mg, 0.3 mmol, 1 equiv) and TFA (5 mL) in DCM (10 mL) was stirred for 1 h at room temperature. The mixture was concentrated under reduced pressure. The residue was dissolved in 7 M NH₃ solution in MeOH (10 mL). The resulting mixture was stirred for 0.5 h. The resulting solution was concentrated under reduced pressure. The crude product was purified with Flash-Prep-HPLC with the following conditions: Column, welch Vltimate XB-C18, 50×250 mm, 10 μm, mobile phase, 0.1% FA and MeCN=5% increasing from 10% to 30% in 10 min. It gave 6-[2,6-difluoro-3-(5-fluoro-2-methylpyridine-3-sulfonamido)phenyl]-7-fluoro-N-methyl-1H-indazole-3-carboxamide (44 mg, 31%) as a white solid. LCMS (ES, m/z): [M+H]⁺: 494. ¹H NMR (300 MHz, DMSO-d₆) δ 14.26 (s, 1H), 8.57 (d, J=2.9 Hz, 1H), 8.49 (d, J=5.0 Hz, 1H), 8.03 (d, J=8.4 Hz, 1H), 7.90 (dd, J=8.4, 2.9 Hz, 1H), 7.32 (q, J=8.8 Hz, 1H), 7.11 (dd, J=14.7, 8.0 Hz, 2H), 2.84 (d, J=4.7 Hz, 3H), 2.76 (d, J=1.2 Hz, 3H).

### Example 66—Synthesis of 6-[2,6-Difluoro-3-(5-fluoro-2-methylpyridine-3-sulfonamido)phenyl]-7-fluoro-N-methyl-1H-pyrazolo[4,3-c]pyridine-3-carboxamide

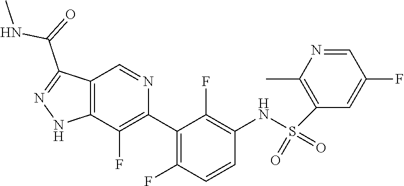 [SMILES: CNC(=O)c1n[nH]c2c(F)c(-c3c(F)ccc(NS(=O)(=O)c4cc(F)cnc4C)c3F)ncc12]

### Part I—Synthesis of ethyl 4-fluoro-3-oxobutanoate

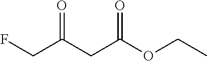

To a stirred solution of ethyl acetate (131.5 g, 1.5 mol, 1.1 equiv) in tetrahydrofuran (200 mL) was added LDA (815 mL, 1.6 mol, 1.2 equiv, 2M in THF) dropwise at −78 degrees C. under nitrogen atmosphere. The resulting mixture was stirred for 0.5 h at −78 degrees C. Ethyl 2-fluoroacetate (144 g, 1.4 mol, 1 equiv) was added dropwise and the resulting mixture was stirred for additional 3 h at −78 degrees C. The reaction was quenched by the addition of 1M HCl (1.6 L) at room temperature. The resulting mixture was extracted with EA (3×1.5 L). The combined organic layers were washed with brine (1×1 L), dried over anhydrous sodium sulfate. After filtration, the filtrate was concentrated under reduced pressure. The residue was purified by silica gel column chromatography, eluted with PE to afford ethyl 4-fluoro-3-oxobutanoate (206.2 g, crude) as red oil.

### Part II—Synthesis of ethyl (2Z)-2-[(dimethylamino)methylidene]-4-fluoro-3-oxobutanoate

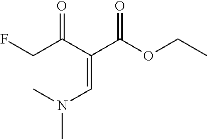

A mixture of ethyl 4-fluoro-3-oxobutanoate (190.0 g, 1.28 mol, 1.00 equiv) and DMF-DMA (168.1 g, 1.41 mol, 1.10 equiv) was stirred for overnight at room temperature. The resulting mixture was concentrated under reduced pressure. The residue was purified by silica gel column chromatography, eluted with PE:EA (2:1) to afford ethyl (2Z)-2-[(dimethylamino)methylidene]-4-fluoro-3-oxobutanoate (175.1 g, 67.1%) as a red oil.

### Part III—Synthesis of 2,6-difluoro-3-nitrobenzoyl chloride

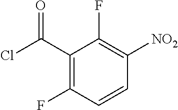 [SMILES: O=C(Cl)c1c(F)ccc([N+](=O)[O-])c1F]

A solution of 2,6-difluoro-3-nitrobenzoic acid (75 g, 369 mmol, 1 equiv) in thionyl chloride (300 mL) was stirred for overnight at 80 degrees C. The mixture was concentrated under vacuum to give 2,6-difluoro-3-nitrobenzoyl chloride (80 g, 98%) as a brown oil.

### Part IV—Synthesis of ethyl 6-(2,6-difluoro-3-nitrophenyl)-5-fluoro-4-oxo-1H-pyridine-3-carboxylate

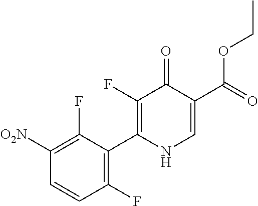 [SMILES: CCOC(=O)c1c[nH]c(-c2c(F)ccc([N+](=O)[O-])c2F)c(F)c1=O]

To a stirred solution ethyl (2Z)-2-[(dimethylamino)methylidene]-4-fluoro-3-oxobutanoate (15 g, 74 mmol, 1 equiv) in tetrahydrofuran (150 mL) under nitrogen atmosphere was added LiHMDS (150 mL, 798 mmol, 10.8 equiv) dropwise at −78 degrees C. The resulting mixture was stirred for 0.5 h at −78 degrees C. then a solution of 2,6-difluoro-3-nitrobenzoyl chloride (19.6 g, 88.6 mmol, 1.2 equiv) in tetrahydrofuran (75 mL) was added dropwise over 0.5 h at −78 degrees C. The resulting mixture was stirred for additional 3 min then a solution of NH₄OAc (8.5 g, 110.7 mmol, 1.5 equiv) in acetic acid (150 mL) was added dropwise over 10 min at −70 degrees C. The reaction mixture was stirred for 0.5 h at room temperature, then concentrated under reduced pressure to remove the solvent. The resulting mixture was heated for 1.5 h at 60 degrees C., then cooled and quenched by the addition of H₂O (300 mL). This was extracted with EA (3×200 mL). The combined organic layers were washed with saturated aqueous NaHCO₃ (3×200 mL), brine (1×300 mL), dried over anhydrous sodium sulfate. After filtration, the filtrate was concentrated under reduced pressure. The residue was purified by silica gel column chromatography, eluted with DCM:MeOH (10:1) to afford ethyl 6-(2,6-difluoro-3-nitrophenyl)-5-fluoro-4-oxo-1H-pyridine-3-carboxylate (7.7 g, 30% yield) as a yellow oil. LCMS (ES, m/z): [M+H]⁺: 343.

### Part V—Synthesis of ethyl 4-chloro-6-(2,6-difluoro-3-nitrophenyl)-5-fluoropyridine-3-carboxylate

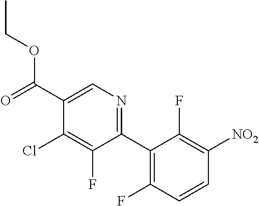 [SMILES: CCOC(=O)c1cnc(-c2c(F)ccc([N+](=O)[O-])c2F)c(F)c1Cl]

A solution of ethyl 6-(2,6-difluoro-3-nitrophenyl)-5-fluoro-4-oxo-1H-pyridine-3-carboxylate (24 g, 70 mmol, 1 equiv) in phosphorus oxychloride (100 mL) was stirred for 2 h at 110 degrees C. The mixture was concentrated under reduced pressure. The residue was basified to pH 9 with saturated aqueous NaHCO₃. The resulting mixture was extracted with EA (3×100 mL). The combined organic layers were washed with brine (2×100 mL), dried over anhydrous sodium sulfate. After filtration, the filtrate was concentrated under reduced pressure. The residue was purified by silica gel column chromatography, eluted with PE:EA (9:1) to afford ethyl 4-chloro-6-(2,6-difluoro-3-nitrophenyl)-5-fluoropyridine-3-carboxylate (6.1 g, 24% yield) as a light yellow oil. LCMS (ES, m/z): [M+H]⁺: 361.

### Part VI—Synthesis of [4-chloro-6-(2,6-difluoro-3-nitrophenyl)-5-fluoropyridin-3-yl]methanol

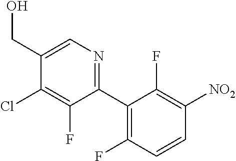 [SMILES: O=[N+]([O-])c1ccc(F)c(-c2ncc(CO)c(Cl)c2F)c1F]

To a stirred solution of ethyl 4-chloro-6-(2,6-difluoro-3-nitrophenyl)-5-fluoropyridine-3-carboxylate (10 g, 28 mmol, 1 equiv) in tetrahydrofuran (300 mL) was added LiAlH₄ (1.2 g, 31.9 mmol, 1.15 equiv) in portions at 0 degrees C. The resulting mixture was stirred for 3 h at 0 degrees C. The reaction was quenched with ice-water (50 mL) and acidified to pH 3 with 1 mol/L HCl. The resulting mixture was extracted with EA (3×200 mL). The combined organic layers were washed with brine (2×100 mL), dried over anhydrous sodium sulfate. After filtration, the filtrate was concentrated under reduced pressure. The residue was purified by silica gel column chromatography, eluted with PE:EA (5:1) to afford [4-chloro-6-(2,6-difluoro-3-nitrophenyl)-5-fluoropyridin-3-yl]methanol (5.5 g, 62% yield) as a yellow solid. LCMS (ES, m/z): [M+H]⁺: 319.

### Part VII—Synthesis of [6-(3-amino-2,6-difluorophenyl)-4-chloro-5-fluoropyridin-3-yl]methanol

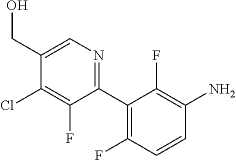 [SMILES: Nc1ccc(F)c(-c2ncc(CO)c(Cl)c2F)c1F]

A solution of [4-chloro-6-(2,6-difluoro-3-nitrophenyl)-5-fluoropyridin-3-yl]methanol (5.5 g, 18.3 mmol, 1 equiv) and Rh/C (5.5 g, 54 mmol, 3 equiv) in EA (100 mL) was stirred for 2 h at room temperature under H₂ (5 atm) atmosphere. The resulting mixture was filtered and the filter cake was washed with EA (3×20 mL). The combined filtrates were concentrated under reduced pressure to afford [6-(3-amino-2,6-difluorophenyl)-4-chloro-5-fluoropyridin-3-yl]methanol (5.1 g, crude) as a yellow oil which was used directly in the next step without further purification. LCMS (ES, m/z): [M+H]⁺: 289.

### Part VIII—Synthesis of 6-(3-amino-2,6-difluorophenyl)-4-chloro-5-fluoropyridine-3-carbaldehyde

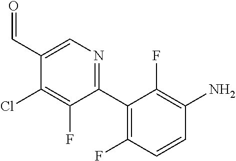 [SMILES: Nc1ccc(F)c(-c2ncc(C=O)c(Cl)c2F)c1F]

A solution of [6-(3-amino-2,6-difluorophenyl)-4-chloro-5-fluoropyridin-3-yl]methanol (5 g, 17.4 mmol, 1 equiv) and MnO₂ (15.1 g, 174 mmol, 10 equiv) in CHCl₃ (80 mL) was stirred for 2 h at 60 degree C. The resulting mixture was filtered and the filter cake was washed with EA (3×10 mL). The combined filtrates were concentrated under reduced pressure to afford 6-(3-amino-2,6-difluorophenyl)-4-chloro-5-fluoropyridine-3-carbaldehyde (3.5 g crude) as a yellow solid which was used directly in the next step without further purification.

### Part IX—Synthesis of 2,4-difluoro-3-[7-fluoro-1H-pyrazolo[4,3-c]pyridin-6-yl]aniline

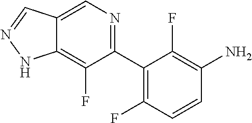 [SMILES: Nc1ccc(F)c(-c2ncc3cn[nH]c3c2F)c1F]

A solution of 6-(3-amino-2,6-difluorophenyl)-4-chloro-5-fluoropyridine-3-carbaldehyde (500 mg, 1.7 mmol, 1 equiv) and hydrazine hydrate (873 mg, 17.4 mmol, 10 equiv) in xylene (5 mL) was stirred for overnight at 120 degrees C. The mixture was concentrated under reduced pressure and the residue purified by silica gel column chromatography, eluting with DCM:CH₃OH (10:1) to afford 2,4-difluoro-3-[7-fluoro-1H-pyrazolo[4,3-c]pyridin-6-yl]aniline (310 mg, 67% yield) as a red solid. LCMS (ES, m/z): [M+H]⁺: 265.

### Part X—Synthesis of 2,4-difluoro-3-[7-fluoro-3-iodo-1H-pyrazolo[4,3-c]pyridin-6-yl]aniline

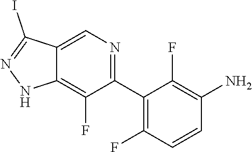 [SMILES: Nc1ccc(F)c(-c2ncc3c(I)n[nH]c3c2F)c1F]

To a stirred mixture of 2,4-difluoro-3-[7-fluoro-1H-pyrazolo[4,3-c]pyridin-6-yl]aniline (1 g, 3.8 mmol, 1 equiv) and KOH (530 mg, 9.5 mmol, 2.5 equiv) in dioxane (10 mL) was added I₂ (1.9 g, 7.6 mmol, 2 equiv) in portions at room temperature. The resulting solution was stirred for 4 hours at 80 degrees C. The mixture was cooled and quenched with water (50 mL). The resulting mixture was extracted with DCM (3×20 mL). The combined organic layers were washed with brine (2×20 mL), dried over anhydrous Na₂SO₄. After filtration, the filtrate was concentrated under reduced pressure to give 2,4-difluoro-3-[7-fluoro-3-iodo-1H-pyrazolo[4,3-c]pyridin-6-yl]aniline (840 mg, crude) as a light yellow solid. LCMS (ES, m/z): [M+H]⁺: 391.

### Part XI—Synthesis of methyl 6-(3-amino-2,6-difluorophenyl)-7-fluoro-1H-pyrazolo[4,3-c]pyridine-3-carboxylate

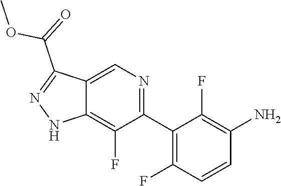 [SMILES: COC(=O)c1n[nH]c2c(F)c(-c3c(F)ccc(N)c3F)ncc12]

To a solution of 2,4-difluoro-3-[7-fluoro-3-iodo-1H-pyrazolo[4,3-c]pyridin-6-yl]aniline (2 g, 5.1 mmol, 1 equiv) in MeOH (50 mL) was added TEA (2.6 g, 26 mmol, 5 equiv) and Pd(dppf)Cl₂ (380 mg, 0.5 mmol, 0.1 equiv) in a pressure tank. The mixture was purged with nitrogen for 3 minutes and then was pressurized to 20 atm with carbon monoxide at 70 degree C. overnight. The reaction mixture was cooled and filtered to remove insoluble solids. The filtrate was concentrated under reduced pressure. The residue was purified by silica gel column chromatography, eluting with DCM:MeOH (10:1) to afford methyl 6-(3-amino-2,6-difluorophenyl)-7-fluoro-1H-pyrazolo[4,3-c]pyridine-3-carboxylate (1.0 g, 61% yield) as a yellow solid. LCMS (ES, m/z): [M+H]⁺: 323.

### Part XII—Synthesis of 6-(3-amino-2,6-difluorophenyl)-7-fluoro-N-methyl-1H-pyrazolo[4,3-c]pyridine-3-carboxamide

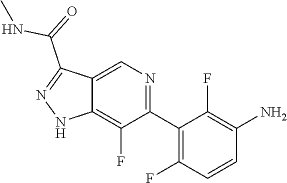 [SMILES: CNC(=O)c1n[nH]c2c(F)c(-c3c(F)ccc(N)c3F)ncc12]

To a stirred mixture of methyl 6-(3-amino-2,6-difluorophenyl)-7-fluoro-1H-pyrazolo[4,3-c]pyridine-3-carboxylate (980 mg, 1 equiv) in THE (10 mL) was added excess methylamine solution in water (10 mL) at room temperature. The resulting solution was stirred for overnight at 50 degree C. The mixture was allowed to cool and was extracted with EA (3×20 mL). The combined organic layers were washed with brine (2×10 mL), dried over anhydrous Na₂SO₄. After filtration, the filtrate was concentrated under reduced pressure to give 6-(3-amino-2,6-difluorophenyl)-7-fluoro-N-methyl-1H-pyrazolo[4,3-c]pyridine-3-carboxamide (890 mg, 91%) as a white solid. LCMS (ES, m/z): [M+H]⁺: 322.

### Part XIII—Synthesis of 6-[2,6-Difluoro-3-(5-fluoro-2-methylpyridine-3-sulfonamido)phenyl]-7-fluoro-N-methyl-1H-pyrazolo[4,3-c]pyridine-3-carboxamide

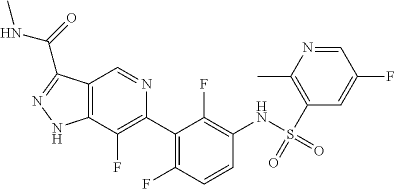 [SMILES: CNC(=O)c1n[nH]c2c(F)c(-c3c(F)ccc(NS(=O)(=O)c4cc(F)cnc4C)c3F)ncc12]

To a stirred solution of 6-(3-amino-2,6-difluorophenyl)-7-fluoro-N-methyl-1H-pyrazolo[4,3-c]pyridine-3-carboxamide (100 mg, 0.3 mmol, 1 equiv) in pyridine (2 mL) was added 5-fluoro-2-methylpyridine-3-sulfonyl chloride (72 mg, 0.34 mmol, 1.1 equiv) in portions at room temperature. The resulting mixture was stirred for 0.5 h then concentrated under vacuum. The residue was purified by prep-HPLC with the following conditions: Column, welch Vltimate XB-C18, 50×250 mm, 10 μm, mobile phase, 0.1% FA and MeCN=5% increasing from 15% to 50% in 10 min. 6-[2,6-Difluoro-3-(5-fluoro-2-methylpyridine-3-sulfonamido)phenyl]-7-fluoro-N-methyl-1H-pyrazolo[4,3-c]pyridine-3-carboxamide (35 mg, 23% yield) was isolated as a white solid. LCMS (ES, m/z): [M+H]⁺: 495. ¹H NMR (300 MHz, DMSO-d₆) δ 14.79 (s, 1H), 10.80 (s, 1H), 9.28 (d, J=2.3 Hz, 1H), 8.72 (d, J=2.9 Hz, 2H), 7.92 (dd, J=8.2, 2.8 Hz, 1H), 7.50 (td, J=8.9, 5.8 Hz, 1H), 7.30 (td, J=8.9, 1.6 Hz, 1H), 2.86 (d, J=4.7 Hz, 3H), 2.77 (d, J=1.3 Hz, 3H).

### Example 67—Synthesis of 6-[3-(5-Chloro-2-methoxypyridine-3-sulfonamido)-2,6-difluorophenyl]-7-fluoro-N-methyl-1H-pyrazolo[4,3-c]pyridine-3-carboxamide

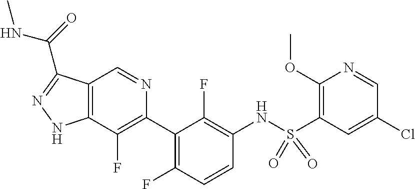 [SMILES: CNC(=O)c1n[nH]c2c(F)c(-c3c(F)ccc(NS(=O)(=O)c4cc(Cl)cnc4OC)c3F)ncc12]

To a stirred mixture of 6-(3-amino-2,6-difluorophenyl)-7-fluoro-N-methyl-1H-pyrazolo[4,3-c]pyridine-3-carboxamide (See Example 67) (60 mg, 0.2 mmol, 1 equiv) and pyridine (89 mg, 1.1 mmol, 6 equiv) in DCM (5 mL) was added a solution of 5-chloro-2-methoxypyridine-3-sulfonyl chloride (68 mg, 0.3 mmol, 1.5 equiv) in DCM (2 mL) dropwise at 0 degree C. The resulting solution was stirred for 2 h at room temperature, then concentrated under reduced pressure. The residue was purified by prep-HPLC with the following conditions: Column, welch Vltimate XB-C18, 50×250 mm, 10 μm, mobile phase, 0.1% FA and MeCN=5% increasing from 20% to 50% in 15 min. 6-[3-(5-Chloro-2-methoxypyridine-3-sulfonamido)-2,6-difluorophenyl]-7-fluoro-N-methyl-1H-pyrazolo[4,3-c]pyridine-3-carboxamide (42 mg, 43% yield) was isolated as a white solid. LCMS (ES, m/z): [M−H]⁻: 525. ¹H NMR (300 MHz, DMSO-d₆) δ 14.77 (s, 1H), 10.46 (s, 1H), 9.29 (d, J=2.2 Hz, 1H), 8.71 (d, J=4.9 Hz, 1H), 8.49 (d, J=2.6 Hz, 1H), 8.08 (d, J=2.6 Hz, 1H), 7.56-7.42 (m, 1H), 7.27 (t, J=8.9 Hz, 1H), 3.90 (s, 3H), 2.86 (d, J=4.7 Hz, 3H).

### Example 68—Synthesis of N-[2,4-difluoro-3-[7-fluoro-3-(hydrazinecarbonyl)-1H-indazol-6-yl]phenyl]-1-benzofuran-6-sulfonamide

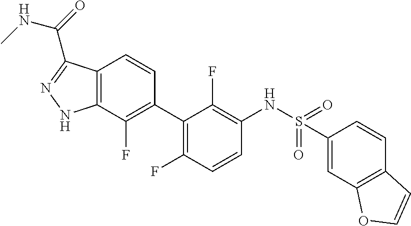 [SMILES: CNC(=O)c1n[nH]c2c(F)c(-c3c(F)ccc(NS(=O)(=O)c4ccc5ccoc5c4)c3F)ccc12]

Into a 8-mL round-bottom flask, was placed 6-[3-(1-benzofuran-6-sulfonamido)-2,6-difluorophenyl]-7-fluoro-N-methyl-1-[[2-(trimethylsilyl)ethoxy]methyl]indazole-3-carboxamide (See Example 62) (100 mg) in DCM (3 mL). This was followed by the addition of TFA (1.00 mL) dropwise with stirring at 0 degrees C. The resulting solution was stirred for 3 hr at room temperature. The reaction was then quenched by the addition of 5 mL of water. The pH was adjusted to 8 with NaHCO₃ (2 mol/L). The resulting solution was extracted with 3×5 mL of dichloromethane. The resulting mixture was washed with 1×5 ml of brine. The mixture was dried over anhydrous sodium sulfate and concentrated under vacuum. The crude product (150 mg) was purified by Prep-HPLC with the following conditions: Water (0.1% FA) and ACN (40% PhaseB up to 60% in 11 min. N-[2,4-difluoro-3-[7-fluoro-3-(hydrazinecarbonyl)-1H-indazol-6-yl]phenyl]-1-benzofuran-6-sulfonamide (23 mg) was isolated as a white solid. LCMS (ES, m/z): [M+H]⁺: 501. ¹H NMR (300 MHz, DMSO-d₆) δ 14.27 (s, 1H), 8.49 (q, J=5.2, 4.7 Hz, 1H), 8.23 (d, J=2.2 Hz, 1H), 7.99 (d, J=8.4 Hz, 1H), 7.93 (d, J=1.5 Hz, 1H), 7.83 (d, J=8.2 Hz, 1H), 7.63 (dd, J=8.2, 1.6 Hz, 1H), 7.36 (td, J=9.0, 5.9 Hz, 1H), 7.17 (t, J=8.9 Hz, 1H), 7.10 (dd, J=2.3, 1.0 Hz, 1H), 6.99 (dd, J=8.4, 5.8 Hz, 1H), 2.83 (d, J=4.7 Hz, 3H).

### Example 69—Synthesis of 6-[2,6-Difluoro-3-(1-methyl-1,3-benzodiazole-4-sulfonamido)phenyl]-7-fluoro-N-methyl-1H-indazole-3-carboxamide

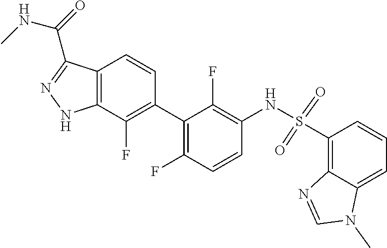 [SMILES: CNC(=O)c1n[nH]c2c(F)c(-c3c(F)ccc(NS(=O)(=O)c4cccc5c4ncn5C)c3F)ccc12]

### Part I—Synthesis of 3-methyl-1,3-benzodiazole-4-sulfonyl chloride

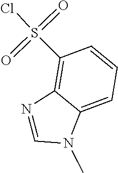

Into a 40-mL vial, was placed MeCN (7.5 mL), 2N HCl (1.5 mL). This was followed by the addition of NCS (945 mg, 7.1 mmol, 4 equiv), in portions at 0 degrees C. To this was added 4-(benzylsulfanyl)-1-methyl-1,3-benzodiazole (See Example 64) (450 mg, 1.8 mmol, 1 equiv) at 0 degrees C., after 10 min stirring. The resulting solution was stirred for 1 hr at 0 degrees C. in a water/ice bath. The reaction was then quenched by the addition of 20 mL of water/ice. The resulting solution was extracted with 2×20 mL of dichloromethane, and the extracts dried over anhydrous sodium sulfate and concentrated. This resulted in 600 mg (88%) of 3-methyl-1,3-benzodiazole-4-sulfonyl chloride as yellow oil.

### Part II—Synthesis of 6-[2,6-Difluoro-3-(1-methyl-1,3-benzodiazole-4-sulfonamido)phenyl]-7-fluoro-N-methyl-1-[[2-(trimethylsilyl)ethoxy]methyl]indazole-3-carboxamide

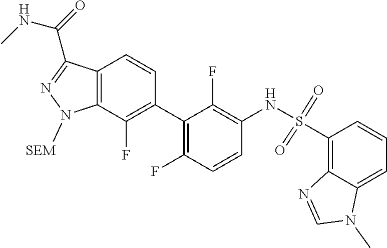 [SMILES: CNC(=O)c1nn(COCC[Si](C)(C)C)c2c(F)c(-c3c(F)ccc(NS(=O)(=O)c4cccc5c4ncn5C)c3F)ccc12]

Into a 40-mL vial, was placed 6-(3-amino-2,6-difluorophenyl)-7-fluoro-N-methyl-1-[[2-(trimethylsilyl)ethoxy] methyl]indazole-3-carboxamide (400 mg, 0.9 mmol, 1 equiv), DCM (9 mL), pyridine (1.4 g, 17.8 mmol, 20 equiv) and 1-methyl-1,3-benzodiazole-4-sulfonyl chloride (614 mg, 2.7 mmol, 3 equiv). The resulting solution was stirred for 1 hr at 25 degrees C. The resulting mixture was concentrated. The crude product was purified by Flash-Prep-HPLC with the following conditions: Column, C18 silica gel; mobile phase, 0.1% NH₃.H₂O and MeCN 5% increasing to 80% within 20 min; Detector, 220 nm. 6-[2,6-Difluoro-3-(1-methyl-1,3-benzodiazole-4-sulfonamido)phenyl]-7-fluoro-N-methyl-1-[[2-(trimethylsilyl)ethoxy]methyl]indazole-3-carboxamide (130 mg, 23% yield) was isolated as a yellow solid. LCMS (ES, m/z): [M+H]⁺: 645.

### Part III—Synthesis of 6-[2,6-Difluoro-3-(1-methyl-1,3-benzodiazole-4-sulfonamido)phenyl]-7-fluoro-N-methyl-1H-indazole-3-carboxamide

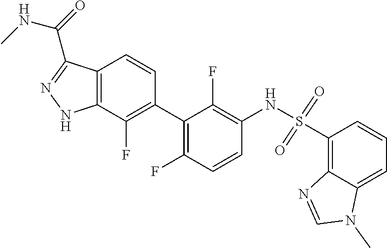 [SMILES: CNC(=O)c1n[nH]c2c(F)c(-c3c(F)ccc(NS(=O)(=O)c4cccc5c4ncn5C)c3F)ccc12]

Into a 50-mL round-bottom flask, was placed 6-[2,6-difluoro-3-(1-methyl-1,3-benzodiazole-4-sulfonamido)phenyl]-7-fluoro-N-methyl-1-[[2-(trimethylsilyl)ethoxy]methyl] indazole-3-carboxamide (130 mg, 0.2 mmol, 1 equiv), DCM (5 mL), and TFA (2 mL). The resulting solution was stirred for 2 hr at 25 degrees C. then concentrated. The pH was adjusted to 8 with NH₃ (7N in MeOH). The crude product was purified by Prep-HPLC with the following conditions: Column, Atlantis Prep T3 OBD Column, 19*150 mm 5 μm; mobile phase, Water (20 MMOL/L NH₄HCO₃) and ACN (30% Phase B up to 50% in 10 min); Detector, uv 220 nm. 6-[2,6-Difluoro-3-(1-methyl-1,3-benzodiazole-4-sulfonamido)phenyl]-7-fluoro-N-methyl-1H-indazole-3-carboxamide (70 mg, 67% yield) was isolated as an off-white solid. LCMS (ES, m/z): [M+H]⁺: 515 [M+H]⁺. ¹H NMR (300 MHz, DMSO-d₆) δ 14.35 (s, 1H), 8.49 (d, J=4.8 Hz, 1H), 8.39 (s, 1H), 8.02 (d, J=8.4 Hz, 1H), 7.91 (dd, J=8.1, 1.0 Hz, 1H), 7.64 (dd, J=7.6, 1.0 Hz, 1H), 7.44-7.26 (m, 2H), 7.13 (dd, J=9.9, 8.3 Hz, 1H), 7.08-6.97 (m, 1H), 3.91 (s, 3H), 2.84 (d, J=4.7 Hz, 3H).

### Example 70—Synthesis of 6-[2,6-Difluoro-3-(5-fluoro-2-methoxypyridine-3-sulfonamido)phenyl]-7-fluoro-N-methyl-1H-pyrazolo[4,3-c]pyridine-3-carboxamide

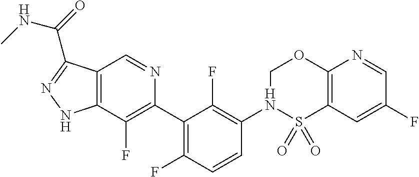

To a stirred mixture of 6-(3-amino-2,6-difluorophenyl)-7-fluoro-N-methyl-1H-pyrazolo[4,3-c]pyridine-3-carboxamide (85 mg, 0.3 mmol, 1 equiv) and pyridine (105 mg, 1.3 mmol, 5 equiv) in DCM (5 mL) was added a solution of 5-fluoro-2-methoxypyridine-3-sulfonyl chloride (90 mg, 0.4 mmol, 1.5 equiv) in DCM (2 mL) dropwise at 0 degree C. The resulting solution was stirred for 2 h at room temperature, then concentrated under reduced pressure. The residue was purified by prep-HPLC with the following conditions (IntelFlash-1): Column, WelFlash TM C18-I, Spherical C18 20-40 m, 120 g; mobile phase, 0.1% FA and MeCN, from 20% increasing to 45% in 15 min; Detector, 220 nm. 6-[2,6-Difluoro-3-(5-fluoro-2-methoxypyridine-3-sulfonamido)phenyl]-7-fluoro-N-methyl-1H-pyrazolo[4,3-c]pyridine-3-carboxamide (53 mg, 39%) was isolated as a white solid. LCMS (ES, m/z): [M+H]⁺: 511. ¹H NMR (300 MHz, DMSO-d₆) δ 14.79 (s, 1H), 10.45 (s, 1H), 9.29 (d, J=2.3 Hz, 1H), 8.71 (d, J=4.8 Hz, 1H), 8.45 (d, J=3.0 Hz, 1H), 8.02 (dd, J=7.3, 3.0 Hz, 1H), 7.49 (td, J=8.9, 5.9 Hz, 1H), 7.27 (t, J=8.9 Hz, 1H), 3.90 (s, 3H), 2.86 (d, J=4.7 Hz, 3H).

### Example 71—Synthesis of 6-[3-(5-Cyano-2-methoxypyridine-3-sulfonamido)-2,6-difluorophenyl]-7-fluoro-N-methyl-1H-pyrazolo[4,3-c]pyridine-3-carboxamide

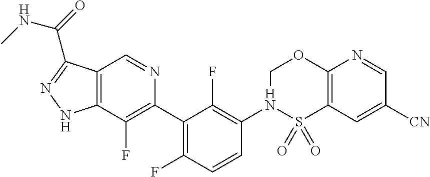

To a stirred mixture of 6-(3-amino-2,6-difluorophenyl)-7-fluoro-N-methyl-1H-pyrazolo[4,3-c]pyridine-3-carboxamide (80 mg, 0.25 mmol, 1 equiv) and pyridine (99 mg, 1.3 mmol, 5 equiv) in DCM (5 mL) was added a solution of 5-cyano-2-methoxypyridine-3-sulfonyl chloride (87 mg, 0.37 mmol, 1.5 equiv) in DCM (2 mL) dropwise at 0 degree C. The resulting solution was stirred for 2 h then concentrated under reduced pressure. The residue was purified by prep-HPLC with the following conditions (IntelFlash-1): Column, WelFlash TM C18-I, Spherical C18 20-40 m, 120 g; mobile phase, 0.1% FA and MeCN, from 20% increasing to 45% in 15 min; Detector, 220 nm. 6-[3-(5-Cyano-2-methoxypyridine-3-sulfonamido)-2,6-difluorophenyl]-7-fluoro-N-methyl-1H-pyrazolo[4,3-c]pyridine-3-carboxamide (63 mg, 49%) was isolated as an off-white solid. LCMS (ES, m/z): [M+H]⁺: 518. ¹H NMR (300 MHz, DMSO-d₆) δ 14.78 (s, 1H), 10.56 (s, 1H), 9.30 (d, J=2.3 Hz, 1H), 8.92 (d, J=2.2 Hz, 1H), 8.71 (d, J=4.8 Hz, 1H), 8.50 (d, J=2.2 Hz, 1H), 7.50 (td, J=8.9, 5.9 Hz, 1H), 7.27 (t, J=9.0 Hz, 1H), 4.00 (s, 3H), 2.86 (d, J=4.6 Hz, 3H).

### Example 72—Synthesis of 6-[3-(5-Chloro-2-methylpyridine-3-sulfonamido)-2,6-difluorophenyl]-7-fluoro-N-methyl-1H-pyrazolo[4,3-c]pyridine-3-carboxamide

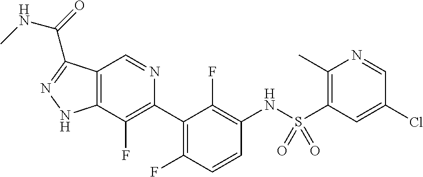 [SMILES: CNC(=O)c1n[nH]c2c(F)c(-c3c(F)ccc(NS(=O)(=O)c4cc(Cl)cnc4C)c3F)ncc12]

### Part I—Synthesis of 3-Bromo-5-chloro-2-methylpyridine

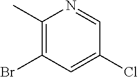

Into a 100-mL round-bottom flask purged and maintained with an inert atmosphere of nitrogen, was placed 2,3-dibromo-5-chloropyridine (5 g, 18.4 mmol, 1 equiv), dioxane (90 mL), K₂CO₃ (7.6 g, 55 mmol, 3 equiv), Pd(PPh₃)₄ (2.1 g, 1.8 mmol, 0.1 equiv) and trimethyl-1,3,5,2,4,6-trioxatriborinane (2.3 g, 18.6 mmol, 1.01 equiv). The resulting solution was stirred for 3 days at 110 degrees C., then cooled and concentrated. The residue was applied onto a silica gel column with ethyl acetate/petroleum ether (1:10). 3-Bromo-5-chloro-2-methylpyridine (2 g, 53% yield) was obtained as an off-white solid. LCMS (ES, m/z): [M+H]⁺: 205.

### Part II—Synthesis of 3-(Benzylsulfanyl)-5-chloro-2-methylpyridine

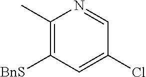

Into a 50-mL round-bottom flask purged and maintained with an inert atmosphere of nitrogen, was placed 3-bromo-5-chloro-2-methylpyridine (850 mg, 4.1 mmol, 1 equiv), dioxane (20 mL), DIEA (1.06 g, 8.2 mmol, 2 equiv), Xantphos (476 mg, 0.8 mmol, 0.2 equiv), Pd₂(dba)₃ (377 mg, 0.4 mmol, 0.1 equiv) and benzyl mercaptan (767 mg, 6.2 mmol, 1.5 equiv). The resulting solution was stirred for 2 h at 100 degrees C., then was cooled and filtered. The filtrate was concentrated and the residue applied to a silica gel column, eluting with ethyl acetate/petroleum ether (1:20). 3-(Benzylsulfanyl)-5-chloro-2-methylpyridine (500 mg, 49% yield) was isolated as a yellow solid. LCMS (ES, m/z): [M+H]⁺: 250.

### Part III—Synthesis of 5-chloro-2-methylpyridine-3-sulfonyl chloride

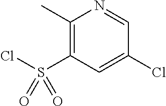

Into a 20-mL vial was placed MeCN (6 mL) and 6M HCl (1.2 mL). This was followed by the addition of NCS (428 mg, 3.2 mmol, 4 equiv), in portions at 0 degrees C. To this mixture was added 3-(benzylsulfanyl)-5-chloro-2-methylpyridine (200 mg, 0.8 mmol, 1 equiv), in portions at 0 degrees C. over 10 mins. The resulting solution was stirred for 30 min at in a water/ice bath. The reaction was quenched by the addition of 20 mL of water, and extracted with 2×20 mL of dichloromethane. The organic layers were combined, washed with 2×20 mL of water. The mixture was dried over anhydrous sodium sulfate and concentrated to give 5-chloro-2-methylpyridine-3-sulfonyl chloride (300 mg) as a crude yellow oil.

### Part IV—Synthesis of 6-[3-(5-Chloro-2-methylpyridine-3-sulfonamido)-2,6-difluorophenyl]-7-fluoro-N-methyl-1H-pyrazolo[4,3-c]pyridine-3-carboxamide

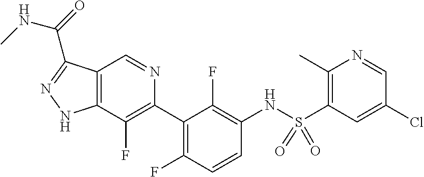 [SMILES: CNC(=O)c1n[nH]c2c(F)c(-c3c(F)ccc(NS(=O)(=O)c4cc(Cl)cnc4C)c3F)ncc12]

To a stirred solution of 6-(3-amino-2,6-difluorophenyl)-7-fluoro-N-methyl-1H-pyrazolo[4,3-c]pyridine-3-carboxamide (See Example 67) (100 mg, 0.3 mmol, 1 equiv) in pyridine (5 mL) was added 5-chloro-2-methylpyridine-3-sulfonyl chloride (106 mg, 0.5 mmol, 1.5 equiv) in portions at room temperature. The resulting mixture was stirred for 0.5 h then concentrated under vacuum. The residue was purified prep-HPLC with the following conditions (IntelFlash-1): Column, WelFlash TM C18-I, Spherical C18 20-40 m, 120 g; mobile phase, 0.1% FA and MeCN, from 35% increasing to 75% in 10 min; Detector, 220 nm. 6-[3-(5-Chloro-2-methylpyridine-3-sulfonamido)-2,6-difluorophenyl]-7-fluoro-N-methyl-1H-pyrazolo[4,3-c]pyridine-3-carboxamide (40 mg, 25%) was isolated as a white solid. LCMS (ES, m/z): [M+H]⁺: 511. ¹H NMR (300 MHz, DMSO-d₆) δ 14.79 (s, 1H), 10.81 (s, 1H), 9.28 (d, J=2.3 Hz, 1H), 8.78-8.68 (m, 2H), 8.05 (d, J=2.4 Hz, 1H), 7.57-7.43 (m, 1H), 7.29 (t, J=9.1 Hz, 1H), 2.90-2.82 (m, 3H), 2.76 (s, 3H).

### Example 73—Synthesis of 5-chloro-N-[2,4-difluoro-3-(7-fluoro-1H-indazol-6-yl)phenyl]-2-methoxypyridine-3-sulfonamide

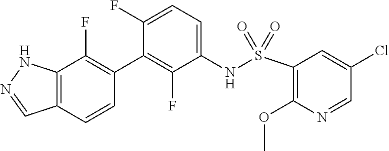 [SMILES: COc1ncc(Cl)cc1S(=O)(=O)Nc1ccc(F)c(-c2ccc3cn[nH]c3c2F)c1F]

### Part I—Synthesis of 5-chloro-N-(2,4-difluoro-3-(7-fluoro-1-((2-(trimethylsilyl)ethoxy)methyl)-1H-indazol-6-yl)phenyl)-2-methoxypyridine-3-sulfonamide

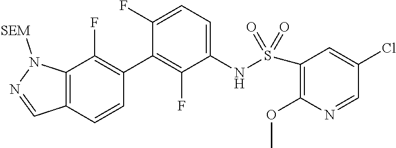 [SMILES: COc1ncc(Cl)cc1S(=O)(=O)Nc1ccc(F)c(-c2ccc3cnn(COCC[Si](C)(C)C)c3c2F)c1F]

6-(3-(5-chloro-2-methoxypyridine-3-sulfonamido)-2,6-difluorophenyl)-7-fluoro-1-((2-(trimethylsilyl)ethoxy)methyl)-1H-indazole-3-carboxylic acid (See Example 48) (200 mg, 0.31 mmol, 1.00 equiv) was dissolved in PhOPh (5.00 mL). The resulting solution was stirred for 0.5 h at 205 degrees C. in an oil bath. LCMS showed that the reaction was completed. The product was purified by column chromatography over silica gel (PE:EA=5:1) to give the product 5-chloro-N-(2,4-difluoro-3-(7-fluoro-1-((2-(trimethylsilyl)ethoxy)methyl)-1H-indazol-6-yl)phenyl)-2-methoxypyridine-3-sulfonamide (160 mg, 85%) as a white solid. LCMS (ES, m/z): [M+H]⁺: 599.

### Part II—Synthesis of 5-chloro-N-[2,4-difluoro-3-(7-fluoro-1H-indazol-6-yl)phenyl]-2-methoxypyridine-3-sulfonamide

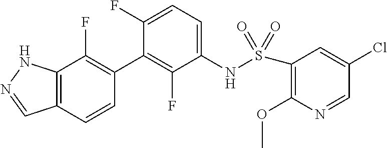 [SMILES: COc1ncc(Cl)cc1S(=O)(=O)Nc1ccc(F)c(-c2ccc3cn[nH]c3c2F)c1F]

A mixture of 5-chloro-N-(2,4-difluoro-3-(7-fluoro-1-((2-(trimethylsilyl)ethoxy)methyl)-1H-indazol-6-yl)phenyl)-2-methoxypyridine-3-sulfonamide (150 mg, 0.25 mmol, 1 equiv) and TFA/DCM (1:4, 5 mL) was stirred for 0.5 hour at room temperature. The resulting mixture was concentrated. The residue was added to ammonia (7.0 M solution in MeOH) (5.00 mL) and stirred for 0.5 hour at room temperature. The resulting solution was concentrated. The residue was purified by prep-HPLC to afford 5-chloro-N-[2,4-difluoro-3-(7-fluoro-1H-indazol-6-yl)phenyl]-2-methoxypyridine-3-sulfonamide (70 mg, yield, 59%) as a white solid. LCMS (ES, m/z): [M+H]⁺: 469. ¹H NMR (300 MHz, DMSO-d₆) δ 13.82 (s, 1H), 10.44 (s, 1H), 8.51 (d, J=2.6 Hz, 1H), 8.27 (d, J=3.5 Hz, 1H), 8.07 (d, J=2.6 Hz, 1H), 7.69 (d, J=8.3 Hz, 1H), 7.45 (td, J=8.9, 5.9 Hz, 1H), 7.27 (td, J=9.0, 1.6 Hz, 1H), 6.99 (dd, J=8.3, 5.8 Hz, 1H), 3.90 (s, 3H).

### Example 74—Synthesis of 6-[3-(cyclopentylmethanesulfonamido)-2,6-difluorophenyl]-7-fluoro-N-methyl-1H-indazole-3-carboxamide

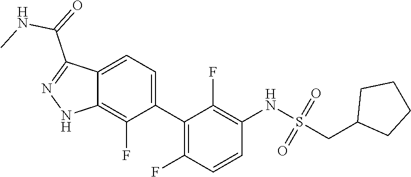 [SMILES: CNC(=O)c1n[nH]c2c(F)c(-c3c(F)ccc(NS(=O)(=O)CC4CCCC4)c3F)ccc12]

### Part I—Synthesis of 6-[3-(cyclopentylmethanesulfonamido)-2,6-difluorophenyl]-7-fluoro-N-methyl-1-[[2-(trimethylsilyl)ethoxy] methyl]indazole-3-carboxamide

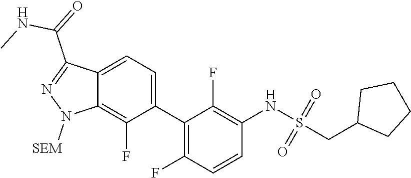 [SMILES: CNC(=O)c1nn(COCC[Si](C)(C)C)c2c(F)c(-c3c(F)ccc(NS(=O)(=O)CC4CCCC4)c3F)ccc12]

To a stirred solution of 6-(3-amino-2,6-difluorophenyl)-7-fluoro-N-methyl-1-[[2-(trimethylsilyl)ethoxy] methyl]indazole-3-carboxamide (250 mg, 0.6 mmol, 1 equiv) in pyridine (2 mL) was added cyclopentylmethanesulfonyl chloride (203 mg, 1.1 mmol, 2 equiv) dropwise at room temperature under nitrogen atmosphere. The resulting mixture was stirred for 3 h at 50 degrees C. under nitrogen atmosphere. The resulting mixture was concentrated under vacuum. The residue was purified by Prep-TLC (PE/EtOAc 1:1) to afford 6-[3-(cyclopentylmethanesulfonamido)-2,6-difluorophenyl]-7-fluoro-N-methyl-1-[[2-(trimethylsilyl)ethoxy] methyl]indazole-3-carboxamide (220 mg, 66%) as a yellow solid. LCMS (ES, m/z): [M+H]⁺: 597.

### Part II—Synthesis of 6-[3-(cyclopentylmethanesulfonamido)-2,6-difluorophenyl]-7-fluoro-N-methyl-1H-indazole-3-carboxamide

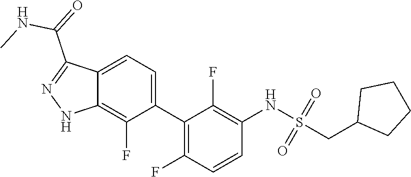 [SMILES: CNC(=O)c1n[nH]c2c(F)c(-c3c(F)ccc(NS(=O)(=O)CC4CCCC4)c3F)ccc12]

To a stirred solution of 6-[3-(cyclopentylmethanesulfonamido)-2,6-difluorophenyl]-7-fluoro-N-methyl-1-[[2-(trimethylsilyl)ethoxy]methyl]indazole-3-carboxamide (210 mg, 0.35 mmol, 1 equiv) in DCM (0.25 mL) was added TFA (0.25 mL, 3.4 mmol, 9.6 equiv) dropwise at room temperature under nitrogen atmosphere. The resulting mixture was stirred for 2 h at room temperature under nitrogen atmosphere. The resulting mixture was concentrated under vacuum and the residue purified by reverse flash chromatography with the following conditions: column, C18; mobile phase, ACN in water, 10% to 65% gradient in 10 min; detector, UV 254 nm to afford 6-[3-(cyclopentylmethanesulfonamido)-2,6-difluorophenyl]-7-fluoro-N-methyl-1H-indazole-3-carboxamide (66.9 mg, 40%) as an off-white solid. LCMS (ES, m/z): [M+H]⁺: 453. ¹H NMR (300 MHz, DMSO-d₆) δ 14.37 (s, 1H), 9.81 (s, 1H), 8.53 (d, J=4.8 Hz, 1H), 8.09 (d, J=8.4 Hz, 1H), 7.56 (td, J=9.0, 5.9 Hz, 1H), 7.36-7.21 (m, 2H), 3.18 (d, J=6.8 Hz, 2H), 2.84 (d, J=4.7 Hz, 3H), 2.28 (p, J=7.8 Hz, 1H), 1.86 (s, 2H), 1.73-1.39 (m, 4H), 1.28 (s, 2H).

### Example 75—Synthesis of 6-[2,6-difluoro-3-(oxane-4-sulfonamido)phenyl]-7-fluoro-N-methyl-1H-indazole-3-carboxamide

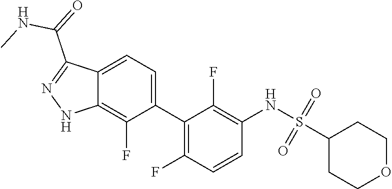 [SMILES: CNC(=O)c1n[nH]c2c(F)c(-c3c(F)ccc(NS(=O)(=O)C4CCOCC4)c3F)ccc12]

### Part I—Synthesis of 6-[2,6-difluoro-3-(oxane-4-sulfonamido)phenyl]-7-fluoro-N-methyl-1-[[2-(trimethylsilyl)ethoxy]methyl]indazole-3-carboxamide

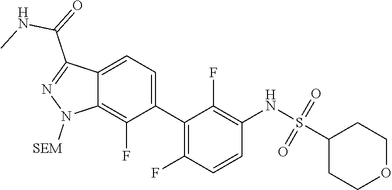 [SMILES: CNC(=O)c1nn(COCC[Si](C)(C)C)c2c(F)c(-c3c(F)ccc(NS(=O)(=O)C4CCOCC4)c3F)ccc12]

To a stirred solution of 6-(3-amino-2,6-difluorophenyl)-7-fluoro-N-methyl-1-[[2-(trimethylsilyl)ethoxy] methyl]indazole-3-carboxamide (250 mg, 0.56 mmol, 1 equiv) in pyridine (5 mL) was added oxane-4-sulfonyl chloride (205 mg, 1.1 mmol, 2 equiv) dropwise at room temperature under nitrogen atmosphere. The resulting mixture was stirred for 3 h at 50 degrees C. under nitrogen atmosphere. The resulting mixture was concentrated under vacuum. The residue was purified by reverse flash chromatography with the following conditions: column, C18; mobile phase, ACN in water, 10% to 60% gradient in 10 min; detector, UV 254 nm to afford 6-[2,6-difluoro-3-(oxane-4-sulfonamido)phenyl]-7-fluoro-N-methyl-1-[[2-(trimethylsilyl)ethoxy]methyl]indazole-3-carboxamide (210 mg, 63%) as a yellow solid. LCMS (ES, m/z): [M+H]⁺: 599.

### Part II—Synthesis of 6-[2,6-difluoro-3-(oxane-4-sulfonamido)phenyl]-7-fluoro-N-methyl-1H-indazole-3-carboxamide

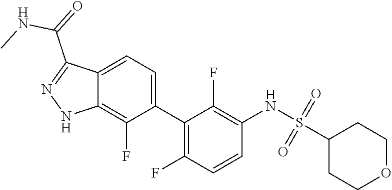 [SMILES: CNC(=O)c1n[nH]c2c(F)c(-c3c(F)ccc(NS(=O)(=O)C4CCOCC4)c3F)ccc12]

To a stirred solution of 6-[2,6-difluoro-3-(oxane-4-sulfonamido)phenyl]-7-fluoro-N-methyl-1-[[2-(trimethylsilyl) ethoxy]methyl]indazole-3-carboxamide (210 mg, 0.35 mmol, 1 equiv) in DCM (0.25 mL) was added TFA (0.25 mL, 3.4 mmol, 9.6 equiv) dropwise at room temperature under nitrogen atmosphere. The resulting mixture was stirred for 1 h at room temperature under nitrogen atmosphere. The resulting mixture was concentrated under vacuum. The crude product was purified by Prep-HPLC with the following conditions (column, C18; mobile phase, ACN in water, 10% to 60% gradient in 10 min; detector, UV 254 nm) to afford 6-[2,6-difluoro-3-(oxane-4-sulfonamido)phenyl]-7-fluoro-N-methyl-1H-indazole-3-carboxamide (72 mg, 43%) as an off-white solid. LCMS (ES, m/z): [M+H]⁺: 469. ¹H NMR (300 MHz, Methanol-d₄) δ 8.13 (d, J=8.4 Hz, 1H), 7.69 (td, J=8.9, 5.7 Hz, 1H), 7.30-7.11 (m, 2H), 4.1-3.98 (m, 2H), 3.42 (td, J=12.0, 2.4 Hz, 3H), 3.01 (s, 3H), 2.07 (d, J=13.0 Hz, 2H), 1.88 (qd, J=12.2, 4.7 Hz, 2H).

### Example 76—Synthesis of 6-[2,6-Difluoro-3-(6-fluoro-1-hydroxy-2,3-dihydro-1H-indene-4-sulfonamido)phenyl]-7-fluoro-N-methyl-1H-indazole-3-carboxamide

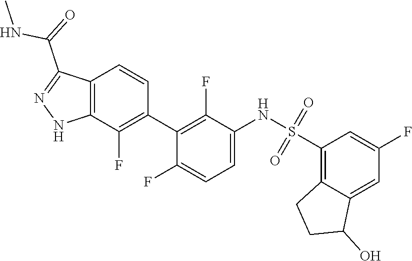 [SMILES: CNC(=O)c1n[nH]c2c(F)c(-c3c(F)ccc(NS(=O)(=O)c4cc(F)cc5c4CCC5O)c3F)ccc12]

### Part I—Synthesis of 4′-(benzylsulfanyl)-6′-fluoro-2′,3′-dihydrospiro[1,3-dioxolane-2,1′-indene]

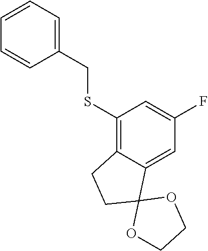 [SMILES: Fc1cc(SCc2ccccc2)c2c(c1)C1(CC2)OCCO1]

Into a 10-mL round-bottom flask was placed 4-(benzylsulfanyl)-6-fluoro-2,3-dihydroinden-1-one (900 mg, 3.3 mmol, 1 equiv), ethylene glycol (4.1 g, 66 mmol, 20 equiv), p-toluene sulfonate (68 mg, 0.4 mmol, 0.12 equiv) and benzene (23 mL). The resulting solution was stirred at reflux overnight under Dean-Stark conditions. The reaction mixture was cooled and diluted with 10 mL of H₂O. The mixture was extracted with 3×30 mL of ethyl acetate, and the extracts dried over anhydrous sodium sulfate. Concentration gave 4′-(benzylsulfanyl)-6′-fluoro-2′,3′-dihydrospiro[1,3-dioxolane-2,1′-indene] (855 mg, 82%) as a yellow solid. LCMS (ES, m/z): [M+H]⁺: 317.

### Part 11—Synthesis of 6-fluoro-1-oxo-2,3-dihydroindene-4-sulfonyl chloride

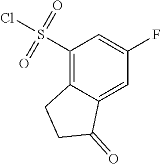 [SMILES: O=C1CCc2c1cc(F)cc2S(=O)(=O)Cl]

Into a 50-mL round-bottom flask, was placed 4′-(benzylsulfanyl)-6′-fluoro-2′,3′-dihydrospiro[1,3-dioxolane-2,1′-indene] (855 mg, 2.7 mmol, 1 equiv), HOAc (9 mL, 252 mmol, 83 equiv), H₂O (3 mL, 167 mmol, 62 equiv) and NCS (1.45 g, 10.8 mmol, 4 equiv). The resulting solution was stirred overnight. The reaction was quenched by the addition of 10 mL water/ice, and extracted with 3×15 mL of ethyl acetate. The extracts were washed with 15 ml of saturated aqueous NaHCO₃ and dried over anhydrous sodium sulfate. Concentration gave a residue which was applied to a silica gel column and eluted with ethyl acetate/petroleum ether (1:2). Concentration of the appropriate fractions gave 6-fluoro-1-oxo-2,3-dihydroindene-4-sulfonyl chloride (430 mg, 64%) as a white solid.

### Part III—Synthesis of 6-[2,6-difluoro-3-(6-fluoro-1-oxo-2,3-dihydroindene-4-sulfonamido)phenyl]-7-fluoro-N-methyl-1-[[2-(trimethylsilyl)ethoxy]methyl]indazole-3-carboxamide

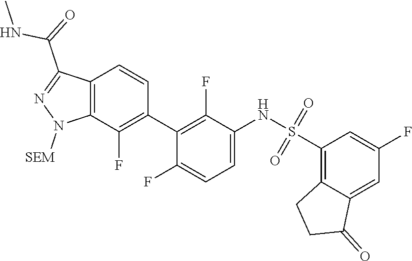 [SMILES: CNC(=O)c1nn(COCC[Si](C)(C)C)c2c(F)c(-c3c(F)ccc(NS(=O)(=O)c4cc(F)cc5c4CCC5=O)c3F)ccc12]

Into a 8-mL vial, was placed 6-(3-amino-2,6-difluorophenyl)-7-fluoro-N-methyl-1-[[2-(trimethylsilyl)ethoxy] methyl]indazole-3-carboxamide (See Example 63) (250 mg, 0.6 mmol, 1 equiv), CH₂Cl₂ (4 mL), pyridine (439 mg, 5.5 mmol, 10 equiv) and 6-fluoro-1-oxo-2,3-dihydroindene-4-sulfonyl chloride (207 mg, 0.83 mmol, 1.5 equiv). The resulting solution was stirred for 30 min, then diluted with 15 mL of H₂O. The resultant mixture was extracted with 3×15 mL of dichloromethane, and the extracts dried over anhydrous sodium sulfate. Concentration gave a residue which was applied to a silica gel column, eluting with ethyl acetate/petroleum ether (1:2). The appropriate fractions were combined and concentrated to give 6-[2,6-difluoro-3-(6-fluoro-1-oxo-2,3-dihydroindene-4-sulfonamido)phenyl]-7-fluoro-N-methyl-1-[[2-(trimethylsilyl)ethoxy]methyl]indazole-3-carboxamide (140 mg, 38%) as a yellow solid. LCMS (ES, m/z): [M+H]⁺: 663.

### Part IV—Synthesis of 6-[2,6-difluoro-3-(6-fluoro-1-oxo-2,3-dihydroindene-4-sulfonamido)phenyl]-7-fluoro-N-methyl-1H-indazole-3-carboxamide

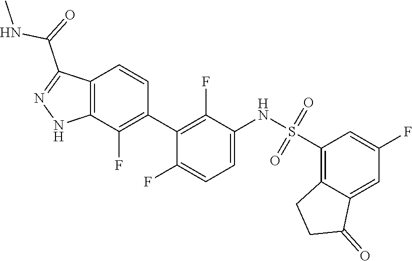 [SMILES: CNC(=O)c1n[nH]c2c(F)c(-c3c(F)ccc(NS(=O)(=O)c4cc(F)cc5c4CCC5=O)c3F)ccc12]

Into a 50-mL round-bottom flask, was placed 6-[2,6-difluoro-3-(6-fluoro-1-oxo-2,3-dihydroindene-4-sulfonamido) phenyl]-7-fluoro-N-methyl-1-[[2-(trimethylsilyl)ethoxy]methyl] indazole-3-carboxamide (140 mg, 1 equiv), CH₂Cl₂ (5 mL) and TFA (5 mL). The resulting solution was stirred overnight, then diluted with 15 mL of H₂O. The pH was adjusted to 8 with saturated aqueous NaHCO₃ and the resulting solution was extracted with 3×15 mL of dichloromethane. The extracts were dried (sodium sulfate), concentrated and the residue applied to a silica gel column, eluting with dichloromethane/petroleum ether (1:3). The appropriate fractions were combined and concentrated to give 6-[2,6-difluoro-3-(6-fluoro-1-oxo-2,3-dihydroindene-4-sulfonamido)phenyl]-7-fluoro-N-methyl-1H-indazole-3-carboxamide (130 mg) as an off-white solid. LCMS (ES, m/z): [M+H]⁺: 533.

### Part V—Synthesis of 6-[2,6-Difluoro-3-(6-fluoro-1-hydroxy-2,3-dihydro-1H-indene-4-sulfonamido)phenyl]-7-fluoro-N-methyl-1H-indazole-3-carboxamide

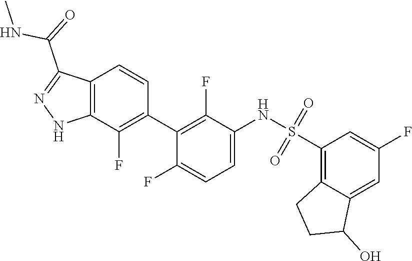 [SMILES: CNC(=O)c1n[nH]c2c(F)c(-c3c(F)ccc(NS(=O)(=O)c4cc(F)cc5c4CCC5O)c3F)ccc12]

Into a 50-mL round-bottom flask, was placed 6-[2,6-difluoro-3-(6-fluoro-1-oxo-2,3-dihydroindene-4-sulfonamido)phenyl]-7-fluoro-N-methyl-1H-indazole-3-carboxamide (124 mg, 0.23 mmol, 1 equiv) and MeOH (20 mL). NaBH₄ (8.8 mg, 0.23 mmol, 1 equiv) was then added at 0 degrees C. The resulting solution was stirred for 30 min at low temperature, then was quenched by the addition of 5 mL water/ice. Extraction with 3×20 mL of ethyl acetate, drying over anhydrous sodium sulfate and concentration gave a residue which was dissolved in 4 mL of CH₃OH. The crude product was purified by Prep-HPLC with the following conditions: Column, Sunfire C18, 30*100 mm, 5 um; mobile phase, water (0.1% FA) and CH₃CN; Gradient: 25% B to 45% in 10 min; Flow rate: 25 mL/min; Detector, 220 nm. 6-[2,6-Difluoro-3-(6-fluoro-1-hydroxy-2,3-dihydro-1H-indene-4-sulfonamido)phenyl]-7-fluoro-N-methyl-1H-indazole-3-carboxamide (30 mg, 24%) was obtained as a white solid. LCMS (ES, m/z): [M+H]⁺: 535. ¹H NMR (300 MHz, MeOD): δ 8.03 (d, J=8.4 Hz, 1H), 7.46 (dd, J=9.1, 2.2 Hz, 1H), 7.37-7.19 (m, 1H), 7.28-6.97 (m, 2H), 6.81 (t, J=8.6 Hz, 1H), 5.16 (s, 1H), 3.00 (s, 3H), 2.34-2.15 (m, 2H), 2.03-1.88 (m, 2H).

### Example 77—Synthesis of 6-[2,6-difluoro-3-(1-methylpiperidine-3-sulfonamido)phenyl]-7-fluoro-N-methyl-1H-indazole-3-carboxamide

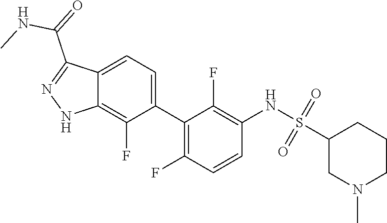 [SMILES: CNC(=O)c1n[nH]c2c(F)c(-c3c(F)ccc(NS(=O)(=O)C4CCCN(C)C4)c3F)ccc12]

### Part I—Synthesis of tert-butyl 3-(methanesulfonyloxy)piperidine-1-carboxylate

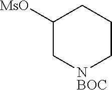

To a stirred mixture of tert-butyl 3-hydroxypiperidine-1-carboxylate (20.1 g, 100 mmol, 1 equiv) and TEA (12.1 g, 120 mmol, 1.2 equiv) in CH₂Cl₂ (400 mL) was added MsCl (12.6 g, 110 mmol, 1.1 equiv) dropwise at 0 degrees C. under nitrogen atmosphere. The resulting mixture was stirred for additional 1 h at room temperature. The reaction was cooled in an ice bath and quenched with sat. NaHCO₃ (aq., 200 mL). The resulting mixture was diluted with CH₂Cl₂ (200 mL). The aqueous layer was extracted with CH₂Cl₂ (3×100 mL). The combined organic layers were washed with brine (1×200 mL) and dried over anhydrous Na₂SO₄. Concentration yielded tert-butyl 3-(methanesulfonyloxy)piperidine-1-carboxylate (30.1 g, crude) as a light yellow oil. The crude product was used in the next step directly without further purification.

### Part II—Synthesis tert-butyl 3-(acetylsulfanyl)piperidine-1-carboxylate

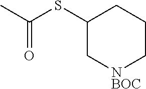

To a stirred solution of tert-butyl 3-(methanesulfonyloxy)piperidine-1-carboxylate (30.1 g, 92 mmol, 1 equiv, 85%) in DMF (500 mL) was added 1-(potassiosulfanyl)ethanone (14.6 g, 128 mmol, 1.4 equiv) in portions at room temperature. The resulting mixture was stirred for 2 h at 100 degrees C. under nitrogen atmosphere. The mixture was allowed to cool and was poured into the water (200 mL), and diluted with EtOAc (500 mL). The aqueous layer was extracted with EtOEt (3×200 mL). The combined organic layers were washed with water (3×200 mL) and brine (1×200 mL), then dried over anhydrous Na₂SO₄. Concentration gave an oil which was purified by silica gel column chromatography, eluting with PE/EtOAc (10:1) to afford tert-butyl 3-(acetylsulfanyl)piperidine-1-carboxylate (7.2 g, 26%) as a yellow oil.

### Part III—Synthesis of tert-butyl 3-(chlorosulfonyl)piperidine-1-carboxylate

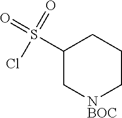

To a stirred solution of tert-butyl 3-(acetylsulfanyl)piperidine-1-carboxylate (5.2 g, 20 mmol, 1 equiv) in MeCN (80 mL) and H₂O (40 mL) was added conc. HCl (1.7 mL, 2.8 equiv) in portions at 0 degrees C. under nitrogen atmosphere. To the above mixture was added NCS (10.7 g, 80 mmol, 4 equiv) in portions over 5 min at 0 degrees C. The resulting mixture was stirred for additional 1 h at room temperature. The reaction was quenched by the addition of Water/Ice (100 mL) and the aqueous layer was extracted with CH₂Cl₂ (3×100 mL). The combined organic layers were washed with brine (2×100 mL), then dried over anhydrous Na₂SO₄. The organics were concentrated under reduced pressure to get tert-butyl 3-(chlorosulfonyl)piperidine-1-carboxylate (6.2 g, crude). The crude product was used in the next step directly without further purification.

### Part IV—Synthesis of tert-butyl 3-[(3-bromo-2,4-difluorophenyl)sulfamoyl]piperidine-1-carboxylate

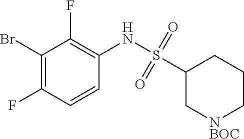

To a stirred mixture of 3-bromo-2,4-difluoroaniline (1.36 g, 6.6 mmol, 0.5 equiv), TEA (2.65 g, 26 mmol, 2 equiv) and DMAP (0.16 g, 1.3 mmol, 0.1 equiv) in CH₂Cl₂ (80 mL) was added tert-butyl 3-(chlorosulfonyl)piperidine-1-carboxylate (6.2 g, 13 mmol, 1 equiv, 60%) in DCM (20 mL) dropwise at 0 degrees C. under nitrogen atmosphere. The resulting mixture was stirred for 1 h at room temperature then poured into ice water (50 mL). The aqueous was extracted with CH₂Cl₂ (3×100 mL). The combined organic layers were washed with brine (1×100 mL), dried over anhydrous Na₂SO₄ and concentrated. The residue was purified by silica gel column chromatography, eluting with PE/EtOAc (10:1 to 5:1) to afford tert-butyl 3-[(3-bromo-2,4-difluorophenyl)sulfamoyl]piperidine-1-carboxylate (980 mg, 15% yield) as a yellow solid. LCMS (ES, m/z): [M+Na]⁺: 477/479.

### Part V—Synthesis of N-(3-bromo-2,4-difluorophenyl)piperidine-3-sulfonamide hydrochloride

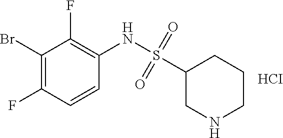

A solution of tert-butyl 3-[(3-bromo-2,4-difluorophenyl)sulfamoyl]piperidine-1-carboxylate (980 mg, 2.2 mmol, 1 equiv) in Et₂O/HCl (20 mL, 2M) was stirred for 1 h at room temperature under nitrogen atmosphere. The resulting mixture was concentrated under reduced pressure to get N-(3-bromo-2,4-difluorophenyl)piperidine-3-sulfonamide hydrochloride (1.0 g, crude). The crude product was used in the next step directly without further purification. LCMS (ES, m/z): [M+H]⁺: 355.

### Part VI—Synthesis of N-(3-bromo-2,4-difluorophenyl)-1-methylpiperidine-3-sulfonamide

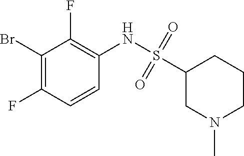 [SMILES: CN1CCCC(S(=O)(=O)Nc2ccc(F)c(Br)c2F)C1]

To a stirred mixture of N-(3-bromo-2,4-difluorophenyl)piperidine-3-sulfonamide hydrochloride (1.00 g, 2 mmol, 1 equiv, 80%) and DIEA (792 mg) in MeOH (20 mL) was added formaldehyde solution (409 mg, 4.1 mmol, 2 equiv, 30%) in one portion at room temperature under nitrogen atmosphere. To this mixture was added NaBH(OAc)₃ (866 mg) in portions over 2 min at room temperature. The resulting mixture was stirred for 1 h at room temperature. The reaction was quenched with water/ice (20 mL) at room temperature. The resulting mixture was diluted with EtOAc (150 mL). The aqueous layer was extracted with EtOAc (3×100 mL) and the combined organic layers were washed with brine (2×50 mL), then dried over anhydrous Na₂SO₄. The filtrate was concentrated under reduced pressure. The residue was purified by silica gel column chromatography, eluting with PE/EtOAc (3:1 to EA) to afford N-(3-bromo-2,4-difluorophenyl)-1-methylpiperidine-3-sulfonamide (420 mg, 50% yield) as a yellow solid. LCMS (ES, m/z): [M+H]⁺: 369.

### Part VII—Synthesis of methyl 6-[2,6-difluoro-3-(1-methylpiperidine-3-sulfonamido)phenyl]-7-fluoro-1-[[2-(trimethylsilyl)ethoxy]methyl]indazole-3-carboxylate

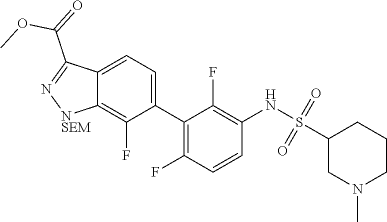 [SMILES: COC(=O)c1nn(COCC[Si](C)(C)C)c2c(F)c(-c3c(F)ccc(NS(=O)(=O)C4CCCN(C)C4)c3F)ccc12]

To a stirred mixture of N-(3-bromo-2,4-difluorophenyl)-1-methylpiperidine-3-sulfonamide (369 mg, 1 mmol, 1 equiv) and methyl 7-fluoro-6-(4,4,5,5-tetramethyl-1,3,2-dioxaborolan-2-yl)-1-[[2-(trimethylsilyl)ethoxy]methyl]indazole-3-carboxylate (See Example 43) (540 mg, 1.2 mmol, 1.2 equiv) in 1,4-dioxane: H₂O=5:1 (5 mL) were added SPhos (164 mg, 0.4 mmol, 0.4 equiv), SPhos Pd Gen.3 (156 mg, 0.2 mmol, 0.2 equiv) and K₂CO₃ (207 mg, 1.5 mmol, 1.5 equiv) in one portion at room temperature under nitrogen atmosphere. The resulting mixture was stirred for 1 h at 80 degrees C. under nitrogen atmosphere. The mixture was allowed to cool to room temperature and was quenched with water (10 mL). The resulting mixture was diluted with EtOAc (50 mL). The aqueous layer was extracted with EtOAc (3×50 mL). The combined organic layers were washed with brine (1×50 mL), then dried over anhydrous Na₂SO₄. The filtrate was concentrated under reduced pressure to give a residue which was purified by silica gel column chromatography, eluting with PE/EtOAc (1:1 to EA) to afford methyl 6-[2,6-difluoro-3-(1-methylpiperidine-3-sulfonamido)phenyl]-7-fluoro-1-[[2-(trimethylsilyl)ethoxy]methyl]indazole-3-carboxylate (590 mg, 96% yield) as a yellow solid. LCMS (ES, m/z): [M+H]⁺: 613.

### Part VIII—Synthesis of 6-[2,6-difluoro-3-(1-methylpiperidine-3-sulfonamido)phenyl]-7-fluoro-N-methyl-1-[[2-(trimethylsilyl)ethoxy]methyl]indazole-3-carboxamide

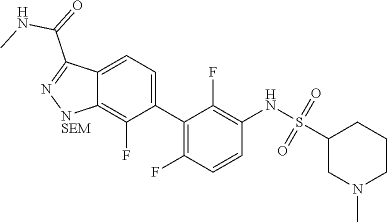 [SMILES: CNC(=O)c1nn(COCC[Si](C)(C)C)c2c(F)c(-c3c(F)ccc(NS(=O)(=O)C4CCCN(C)C4)c3F)ccc12]

To a stirred solution of methyl 6-[2,6-difluoro-3-(1-methylpiperidine-3-sulfonamido)phenyl]-7-fluoro-1-[[2-(trimethylsilyl)ethoxy]methyl]indazole-3-carboxylate (550 mg, 0.9 mmol, 1 equiv) in ethanol (5 mL) was added CH₃NH₂ (15 mL, in alcohol) at room temperature under nitrogen atmosphere. The mixture was stirred 2 h at room temperature. The resulting mixture was concentrated under reduced pressure to give crude 6-[2,6-difluoro-3-(1-methylpiperidine-3-sulfonamido)phenyl]-7-fluoro-N-methyl-1-[[2-(trimethylsilyl)ethoxy]methyl]indazole-3-carboxamide (550 mg). This was used in the next step directly without further purification. LCMS (ES, m/z): [M+H]⁺: 612.

### Part IX—Synthesis of 6-[2,6-difluoro-3-(1-methylpiperidine-3-sulfonamido)phenyl]-7-fluoro-N-methyl-1H-indazole-3-carboxamide

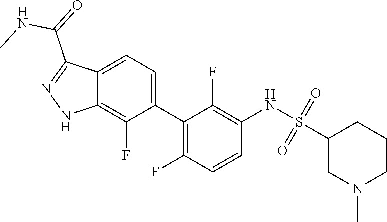 [SMILES: CNC(=O)c1n[nH]c2c(F)c(-c3c(F)ccc(NS(=O)(=O)C4CCCN(C)C4)c3F)ccc12]

To a stirred solution of 6-[2,6-difluoro-3-(1-methylpiperidine-3-sulfonamido)phenyl]-7-fluoro-N-methyl-1-[[2-(trimethylsilyl)ethoxy]methyl]indazole-3-carboxamide (220 mg, 0.31 mmol, 1 equiv, 85%) in CH₂Cl₂ (10 mL) was added TFA (5 mL) in one portion at 0 degrees C. under nitrogen atmosphere. The resulting mixture was stirred for 1 h at room temperature then concentrated. The residue was dissolved in MeOH (2 mL) and was basified to pH 8 with NH₃.CH₃OH (1 mL, 7 M). The crude product was purified by prep-HPLC with the following conditions: Column, welch Vltimate XB-C18, 50×250 mm, 10 μm, mobile phase: Mobile Phase A: 0.1% FA in Water, Mobile Phase B: CAN (10% up to 30% in 10 min) to afford 6-[2,6-difluoro-3-(1-methylpiperidine-3-sulfonamido)phenyl]-7-fluoro-N-methyl-1H-indazole-3-carboxamide (110 mg, 63% yield) as a white solid. LCMS (ES, m/z): [M+H]⁺: 482. ¹H NMR (300 MHz, DMSO-d₆) δ 14.33 (s, 1H), 9.88 (s, 1H), 8.57-8.46 (m, 1H), 8.09 (d, J=8.4 Hz, 1H), 7.57 (td, J=9.0, 5.9 Hz, 1H), 7.34-7.23 (m, 2H), 3.22-3.03 (m, 2H), 2.85 (d, J=4.7 Hz, 3H), 2.70 (d, J=11.3 Hz, 1H), 2.19 (s, 3H), 2.15-1.93 (m, 2H), 1.79 (dd, J=26.3, 13.4 Hz, 2H), 1.47 (t, J=11.7 Hz, 2H).

### Example 78—Synthesis of 6-[2,6-difluoro-3-[(3-hydroxycyclopentyl)methanesulfonamido]phenyl]-7-fluoro-N-methyl-1H-indazole-3-carboxamide

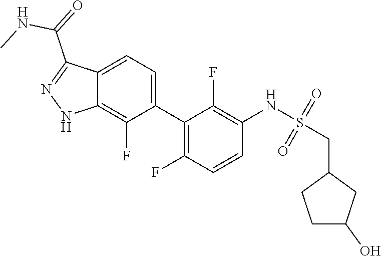 [SMILES: CNC(=O)c1n[nH]c2c(F)c(-c3c(F)ccc(NS(=O)(=O)CC4CCC(O)C4)c3F)ccc12]

### Part I—Synthesis of ethyl 1,4-dioxaspiro[4.4]nonane-7-carboxylate

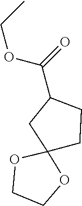

To a stirred mixture of ethyl 3-oxocyclopentane-1-carboxylate (25 g, 160 mmol, 1 equiv) and ethylene glycol (24.8 g, 400 mmol, 2.5 equiv) in toluene (100 mL) was added p-TsOH.H₂O (3.04 g, 16 mmol, 0.1 equiv) in portions at room temperature. The resulting mixture was stirred for 3 h at 130 degrees C. The mixture was allowed to cool and was concentrated under reduced pressure. The residue was purified by silica gel column chromatography, eluting with PE:EA (4:1) to afford ethyl 1,4-dioxaspiro[4.4]nonane-7-carboxylate (9.2 g, 29%) as a colorless oil.

### Part II—Synthesis of 1,4-dioxaspiro[4.4]nonan-7-ylmethanol

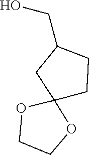 [SMILES: OCC1CCC2(C1)OCCO2]

To a stirred solution of ethyl 1,4-dioxaspiro[4.4]nonane-7-carboxylate (9.1 g, 45 mmol, 1 equiv) in THE (100 mL) was added LAH (2.07 g, 54 mmol, 1.2 equiv) in portions at 0 degrees C. The reaction was stirred for 2 h at low temperature, then quenched with Na₂SO₄.10H₂O (1.0 g). The resulting mixture was stirred for 0.5 h, then filtered. The filtrate was concentrated under reduced pressure to afford 1,4-dioxaspiro[4.4]nonan-7-ylmethanol (6.8 g, 95%) as a yellow oil.

### Part III—Synthesis of 1,4-dioxaspiro[4.4]nonan-7-ylmethyl methanesulfonate

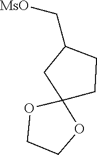 [SMILES: CS(=O)(=O)OCC1CCC2(C1)OCCO2]

To a stirred solution of 1,4-dioxaspiro[4.4]nonan-7-ylmethanol (3.0 g, 19 mmol, 1 equiv) and TEA (3.84 g, 38 mmol, 2 equiv) in DCM (30 mL) were added MsCl (3.3 g, 28 mmol, 1.5 equiv) dropwise at 0 degrees C. The resulting mixture was stirred for 2 h at room temperature. The reaction was quenched with water (100 mL) and extracted with DCM (3×100 mL). The combined organic layers were washed with brine (2×100 mL), dried over anhydrous Na₂SO₄. After filtration, the filtrate was concentrated under reduced pressure to give 1,4-dioxaspiro[4.4]nonan-7-ylmethyl methanesulfonate (5.2 g, crude) as a yellow oil.

### Part IV—Synthesis of 7-[(benzylsulfanyl)methyl]-1,4-dioxaspiro[4.4]nonane

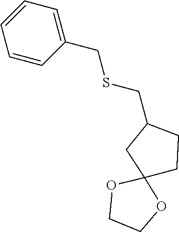 [SMILES: c1ccc(CSCC2CCC3(C2)OCCO3)cc1]

A mixture of 1,4-dioxaspiro[4.4]nonan-7-ylmethyl methanesulfonate (1.5 g, 6.4 mmol, 1 equiv), Cs₂CO₃ (4.14 g, 12.7 mmol, 2 equiv) and benzyl mercaptan (1.18 g, 9.5 mmol, 1.5 equiv) in DMF (30 mL) was stirred for 0.5 h at 100 degrees C. The resulting mixture was filtered and the filter cake was washed with EA (2×100 mL). The filtrate was concentrated under reduced pressure. The residue was purified by reverse flash chromatography with the following conditions: column, C18 silica gel; mobile phase, acetonitrile in water (0.1% NH₄HCO₃ & 0.05% NH₃.H₂O), 15% to 60% gradient in 12 min; detector, UV 220 nm. This resulted in 7-[(benzylsulfanyl)methyl]-1,4-dioxaspiro[4.4]nonane (1.3 g, 80%) as a yellow oil. LCMS (ES, m/z): [M+H]⁺: 265.

### Part V—Synthesis of 3-[(benzylsulfanyl)methyl]cyclopentan-1-one

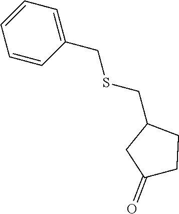 [SMILES: O=C1CCC(CSCc2ccccc2)C1]

A mixture of 7-[(benzylsulfanyl)methyl]-1,4-dioxaspiro[4.4]nonane (900 mg, 3.4 mmol, 1 equiv) and aq. 6 M HCl (10 mL) in THE (10 mL) was stirred for 2 h at room temperature. The resulting mixture was extracted with EA (3×20 mL). The combined organic layers were washed with brine (2×20 mL) and dried over anhydrous Na₂SO₄. After filtration, the filtrate was concentrated under reduced pressure to give 3-[(benzylsulfanyl)methyl]cyclopentan-1-one (830 mg, crude) as a light yellow oil. LCMS (ES, m/z): [M+H]⁺: 221.

### Part VI—Synthesis of 3-[(benzylsulfanyl)methyl]cyclopentan-1-ol

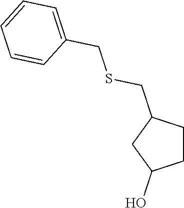 [SMILES: OC1CCC(CSCc2ccccc2)C1]

To a stirred mixture of 3-[(benzylsulfanyl)methyl]cyclopentan-1-one (830 mg, 3.8 mmol, 1 equiv) in MeOH (20 mL) was added NaBH₄ (285 mg, 7.5 mmol, 2 equiv) in portions at 0 degrees C. The resulting solution was stirred for 2 h at room temperature. The reaction was quenched with saturated aqueous NH₄Cl (20 mL) and the resulting mixture was extracted with EA (3×50 mL). The combined organic layers were washed with brine (2×100 mL), then dried over anhydrous Na₂SO₄. After filtration, the filtrate was concentrated under reduced pressure to give 3-[(benzylsulfanyl)methyl]cyclopentan-1-ol (720 mg, 86%) as a yellow oil. LCMS (ES, m/z): [M+H]⁺: 223.

### Part VII—Synthesis of 3-[(benzylsulfanyl)methyl]cyclopentyl acetate

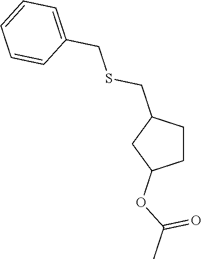 [SMILES: CC(=O)OC1CCC(CSCc2ccccc2)C1]

To a stirred solution of 3-[(benzylsulfanyl)methyl]cyclopentan-1-ol (700 mg, 3.2 mmol, 1 equiv) and TEA (637 mg, 6.3 mmol, 2 equiv) in DCM (20 mL) was added AcCl (371 mg, 4.7 mmol, 1.5 equiv) dropwise at 0 degrees C. The mixture was stirred for 3 h at room temperature. The reaction was quenched with water (10 mL). The resulting solution was extracted with EA (3×50 mL) and the combined organics were washed with brine (2×50 mL), then dried over anhydrous Na₂SO₄. The filtrate was concentrated under reduced pressure. The residue was purified by reverse flash chromatography with the following conditions: column, C18 silica gel; mobile phase, acetonitrile in water (0.1% NH4HCO₃ & 0.05% ammonia), 20% to 70% gradient in 12 min; detector, UV 220 nm. This resulted in 3-[(benzylsulfanyl)methyl]cyclopentyl acetate (280 mg, 34% yield) as a yellow oil. LCMS (ES, m/z): [M+H]⁺: 265.

### Part VIII—Synthesis of 3-[(chlorosulfonyl)methyl]cyclopentyl acetate

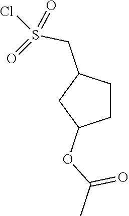

To a stirred solution of 3-[(benzylsulfanyl)methyl]cyclopentyl acetate (500 mg, 1.9 mmol, 1 equiv) and H₂O (1 mL) in HOAc (5 mL) was added NCS (884 mg, 6.6 mmol, 3.5 equiv) in portions at 0 degrees C. The mixture was stirred for 4 h below 20 degrees C. The reaction was quenched with water (20 mL) and extracted with EA (3×20 mL). The combined organic layers were washed with brine (2×20 mL) and dried over anhydrous Na₂SO₄. After filtration, the filtrate was concentrated under reduced pressure to give 3-[(chlorosulfonyl)methyl]cyclopentyl acetate (234 mg, crude) as a yellow oil.

### Part IX—Synthesis of 3-[([2,4-difluoro-3-[7-fluoro-3-(methylcarbamoyl)-1-[[2-(trimethylsilyl)ethoxy] methyl] indazol-6-yl]phenyl]sulfamoyl)methyl]cyclopentyl acetate

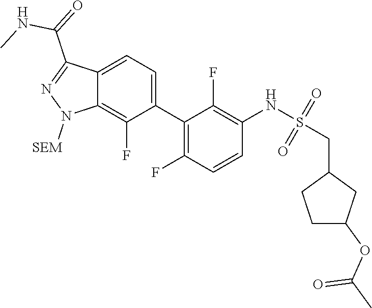 [SMILES: CNC(=O)c1nn(COCC[Si](C)(C)C)c2c(F)c(-c3c(F)ccc(NS(=O)(=O)CC4CCC(OC(C)=O)C4)c3F)ccc12]

To a stirred mixture of 6-(3-amino-2,6-difluorophenyl)-7-fluoro-N-methyl-1-[[2-(trimethylsilyl)ethoxy]methyl] indazole-3-carboxamide (293 mg, 0.65 mmol, 1 equiv) and 3-[(chlorosulfonyl)methyl]cyclopentyl acetate (235 mg, 1 mmol, 1.5 equiv) in DCM (5 mL) was added TEA (197 mg, 2 mmol, 3 equiv) and DMAP (8 mg, 0.06 mmol, 0.1 equiv). The resulting solution was stirred for 4 h at room temperature. The mixture was concentrated under reduced pressure. The residue was purified by reverse flash chromatography with the following conditions: column, C18 silica gel; mobile phase, acetonitrile in water (0.1% NH₄HCO₃ & 0.05% NH₃.H₂O), 20% to 70% gradient in 12 min; detector, UV 254 nm. This resulted in 3-[([2,4-difluoro-3-[7-fluoro-3-(methylcarbamoyl)-1-[[2-(trimethylsilyl)ethoxy]methyl] indazol-6-yl]phenyl]sulfamoyl)methyl]cyclopentyl acetate (182 mg, 43% yield) as a yellow oil. LCMS (ES, m/z): [M+H]⁺: 655.

### Part X—Synthesis of 6-[2,6-difluoro-3-[(3-hydroxycyclopentyl)methanesulfonamido]phenyl]-7-fluoro-N-methyl-1H-indazole-3-carboxamide

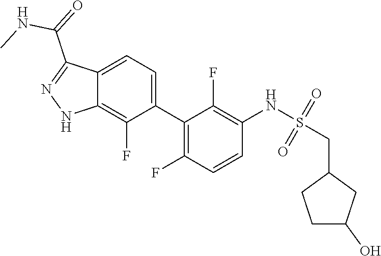 [SMILES: CNC(=O)c1n[nH]c2c(F)c(-c3c(F)ccc(NS(=O)(=O)CC4CCC(O)C4)c3F)ccc12]

A mixture of 3-[([2,4-difluoro-3-[7-fluoro-3-(methylcarbamoyl)-1-[[2-(trimethylsilyl)ethoxy]methyl]indazol-6-yl]phenyl]sulfamoyl)methyl]cyclopentyl acetate (100.00 mg, 0.15 mmol, 1.00 equiv) and TBAF (199.66 mg, 0.76 mmol, 5.00 equiv) in THE (10.00 mL) was stirred for overnight at 60 degrees C. The reaction was quenched with water (20.00 mL). The resulting mixture was extracted with EA (3×50.00 mL). The combined organic layers were washed with brine (2×50.00 mL), dried over anhydrous Na₂SO₄. After filtration, the filtrate was concentrated under reduced pressure. The residue was purified by Prep-HPLC with the following conditions welch Vltimate XB-C18, 50×250 mm, 10 μm, mobile phase: Water (0.1% FA) and ACN (25% Phase B up to 45% in 10 min); Detector, UV 220 nm. This resulted in 6-[2,6-difluoro-3-[(3-hydroxycyclopentyl) methanesulfonamido]phenyl]-7-fluoro-N-methyl-1H-indazole-3-carboxamide (48 mg, 65%, mixture of cis and trans isomers) as a white solid. LCMS (ES, m/z): [M+H]⁺: 483. ¹H NMR (300 MHz, DMSO-d₆) 8.52 (d, J=4.8 Hz, 1H), 8.09 (d, J=8.4 Hz, 1H), 7.60-7.51 (m, 1H), 7.32-7.23 (m, 2H), 4.51-4.41 (m, 1H), 4.08-4.01 (m, 1H), 3.26-3.12 (m, 2H), 2.84 (s, 3H), 2.48-2.31 (m, 1H), 2.16-2.02 (m, 1H), 1.92-1.71 (m, 1H), 1.62-1.21 (m, 4H).

### Example 79—Synthesis of 6-[3-(5-Chloro-2-methoxypyridine-3-sulfonamido)-2,6-difluorophenyl]-N-cyclopentyl-7-fluoro-1H-indazole-3-carboxamide

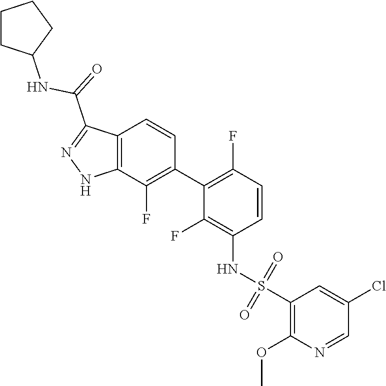 [SMILES: COc1ncc(Cl)cc1S(=O)(=O)Nc1ccc(F)c(-c2ccc3c(C(=O)NC4CCCC4)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), cyclopentanamine (33 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The filtrate was purified by Prep-HPLC with the following conditions: Column, T3, 19*150 mm, Sum; mobile phase, Water (0.1% FA) and CH₃CN (60% CH₃CN up to 75% in 7 min); Detector, 254 nm & 220 nm. 6-[3-(5-Chloro-2-methoxypyridine-3-sulfonamido)-2,6-difluorophenyl]-N-cyclopentyl-7-fluoro-1H-indazole-3-carboxamide (25 mg, 22% yield) was isolated as an off-white solid. LCMS (ES, m/z): [M+H]⁺: 580. ¹H NMR (300 MHz, DMSO-d₆) δ 14.30 (s, 1H), 10.46 (s, 1H), 8.51 (d, J=2.6 Hz, 1H), 8.37 (d, J=7.8 Hz, 1H), 8.15-7.99 (m, 2H), 7.57-7.39 (m, 1H), 7.38-7.23 (m, 1H), 7.22-7.02 (m, 1H), 4.40-4.22 (m, 1H), 3.91 (s, 3H), 2.03-1.83 (m, 2H), 1.83-1.43 (m, 6H).

### Example 80—Synthesis of 6-[3-(5-Chloro-2-methoxypyridine-3-sulfonamido)-2,6-difluorophenyl]-7-fluoro-N-(3-hydroxycyclohexyl)-1H-indazole-3-carboxamide

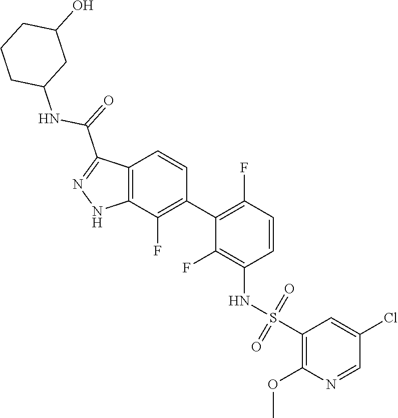 [SMILES: COc1ncc(Cl)cc1S(=O)(=O)Nc1ccc(F)c(-c2ccc3c(C(=O)NC4CCCC(O)C4)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), 3-aminocyclohexan-1-ol (45 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, T3, 19*150 mm, Sum; mobile phase, Water (0.1% FA) and CH₃CN (40% CH₃CN up to 60% in 7 min); Detector, 254 nm & 220 nm. 6-[3-(5-Chloro-2-methoxypyridine-3-sulfonamido)-2,6-difluorophenyl]-7-fluoro-N-(3-hydroxycyclohexyl)-1H-indazole-3-carboxamide (34.4 mg, 29% yield) was isolated as a white solid. LCMS (ES, m/z): [M+H]⁺: 610. ¹H NMR (300 MHz, DMSO-d₆) δ 14.29 (s, 1H), 10.46 (s, 1H), 8.51 (d, J=2.6 Hz, 1H), 8.16 (d, J=8.7 Hz, 1H), 8.11-8.00 (m, 2H), 7.52-7.40 (m, 1H), 7.34-7.23 (m, 1H), 7.18-7.06 (m, 1H), 4.46 (d, J=3.0 Hz, 1H), 4.37-4.20 (m, 1H), 4.05-3.95 (m, 1H), 3.91 (s, 3H), 1.80-1.30 (m, 8H).

### Example 81—Synthesis of 6-[3-(5-Chloro-2-methoxypyridine-3-sulfonamido)-2,6-difluorophenyl]-N-cyclopropyl-7-fluoro-1H-indazole-3-carboxamide

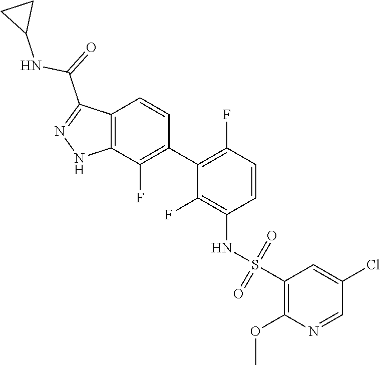 [SMILES: COc1ncc(Cl)cc1S(=O)(=O)Nc1ccc(F)c(-c2ccc3c(C(=O)NC4CC4)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), aminocyclopropane (22 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The mixture was purified by Prep-HPLC with the following conditions: Column, T3, 19*150 mm, Sum; mobile phase, Water (0.1% FA) and CH₃CN (25% CH₃CN up to 85% in 6 min); Detector, 254 nm & 220 nm. 6-[3-(5-Chloro-2-methoxypyridine-3-sulfonamido)-2,6-difluorophenyl]-N-cyclopropyl-7-fluoro-1H-indazole-3-carboxamide (23 mg, 21% yield) was isolated as a grey solid. LCMS (ES, m/z): [M+H]⁺: 552. ¹H NMR (300 MHz, DMSO-d₆): δ 14.30 (s, 1H), 10.46 (s, 1H), 8.59 (d, J=4.6 Hz, 1H), 8.50 (d, J=2.6 Hz, 1H), 8.11-8.01 (m, 2H), 7.46 (td, J=8.9, 5.9 Hz, 1H), 7.34-7.22 (m, 1H), 7.19-7.08 (m, 1H), 3.90 (s, 3H), 3.15-2.55 (m, 1H), 0.69 (td, J=5.6, 2.5 Hz, 4H).

### Example 82—Synthesis of 6-[3-(5-Chloro-2-ethoxypyridine-3-sulfonamido)-2,6-difluorophenyl]-7-fluoro-N-(morpholin-4-ylmethyl)-1H-indazole-3-carboxamide

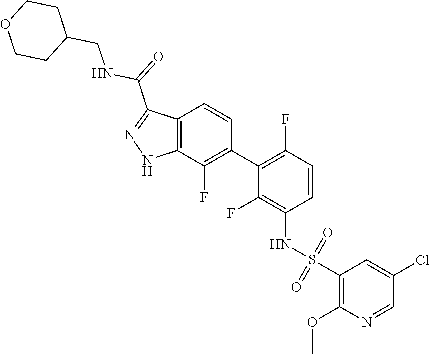 [SMILES: COc1ncc(Cl)cc1S(=O)(=O)Nc1ccc(F)c(-c2ccc3c(C(=O)NCC4CCOCC4)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), 1-(morpholin-4-yl)methanamine (45 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, Xselect Fluoro-Phenyl, 19*150 mm, 5 um; mobile phase, Water (0.1% FA) and CH₃CN (40% CH₃CN up to 57% in 7 min); Detector, 254 nm & 220 nm. 6-[3-(5-Chloro-2-ethoxypyridine-3-sulfonamido)-2,6-difluorophenyl]-7-fluoro-N-(morpholin-4-ylmethyl)-1H-indazole-3-carboxamide (44 mg, 37% yield) was isolated as a white solid. LCMS (ES, m/z): [M+H]⁺: 610. ¹H NMR (300 MHz, DMSO-d₆) δ 14.32 (s, 1H), 8.65-8.55 (m, 1H), 8.50 (d, J=2.6 Hz, 1H), 8.11-8.01 (m, 2H), 7.53-7.40 (m, 1H), 7.34-7.23 (m, 1H), 7.19-7.08 (m, 1H), 3.96-3.78 (m, 5H), 3.30-3.18 (m, 4H), 1.96-1.78 (m, 1H), 1.69-1.54 (m, 2H), 1.32-1.17 (m, 2H).

### Example 83—Synthesis of 6-[3-(5-chloro-2-methoxypyridine-3-sulfonamido)-2,6-difluorophenyl]-7-fluoro-N-[2-(pyrrolidin-1-yl)ethyl]-1H-indazole-3-carboxamide

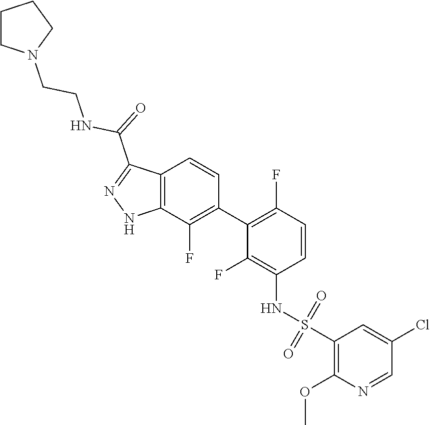 [SMILES: COc1ncc(Cl)cc1S(=O)(=O)Nc1ccc(F)c(-c2ccc3c(C(=O)NCCN4CCCC4)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), 1-pyrrolidineethanamine (45 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, Xselect Fluoro-Phenyl, 19*150 mm, 5 um; mobile phase, Water (0.1% FA) and CH₃CN (15% CH₃CN up to 35% in 6 min); Detector, 254 nm & 220 nm. 6-[3-(5-chloro-2-methoxypyridine-3-sulfonamido)-2,6-difluorophenyl]-7-fluoro-N-[2-(pyrrolidin-1-yl)ethyl]-1H-indazole-3-carboxamide (44 mg, 37% yield) was isolated as a white solid. LCMS (ES, m/z): [M+H]⁺: 609. ¹H NMR (300 MHz, DMSO-d₆) δ 8.71-8.56 (m, 1H), 8.41 (d, J=2.6 Hz, 1H), 8.16 (s, 1H), 8.12-7.95 (m, 2H), 7.46-7.31 (m, 1H), 7.25-7.06 (m, 2H), 3.87 (s, 3H), 3.61-3.53 (m, 2H), 3.06-3.00 (m, 2H), 3.00-2.88 (m, 4H), 1.91-1.76 (m, 4H).

### Example 84—Synthesis of 6-[3-(5-Chloro-2-methoxypyridine-3-sulfonamido)-2,6-difluorophenyl]-7-fluoro-N-(2-methanesulfonylethyl)-1H-indazole-3-carboxamide

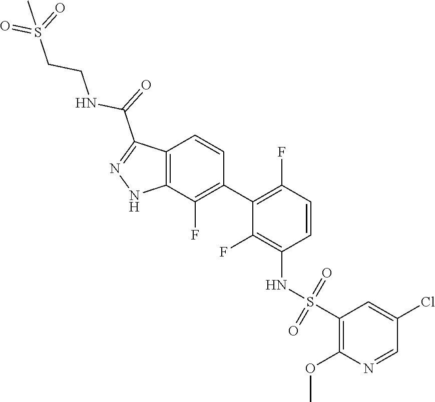 [SMILES: COc1ncc(Cl)cc1S(=O)(=O)Nc1ccc(F)c(-c2ccc3c(C(=O)NCCS(C)(=O)=O)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), 2-methanesulfonylethanamine (48 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, Xselect Fluoro-Phenyl, 19*150 mm, 5 um; mobile phase, Water (0.1% FA) and CH₃CN (33% CH₃CN up to 53% in 7 min); Detector, 254 nm & 220 nm. 6-[3-(5-Chloro-2-methoxypyridine-3-sulfonamido)-2,6-difluorophenyl]-7-fluoro-N-(2-methanesulfonylethyl)-1H-indazole-3-carboxamide (39 mg, 33% yield) was isolated as a white solid. LCMS (ES, m/z): [M+H]⁺: 618. ¹H NMR (300 MHz, DMSO-d₆) δ 14.41 (s, 1H), 8.76 (t, J=5.9 Hz, 1H), 8.51 (d, J=2.6 Hz, 1H), 8.25-7.93 (m, 2H), 7.47 (td, J=8.9, 5.9 Hz, 1H), 7.33-7.22 (m, 1H), 7.25-7.01 (m, 1H), 3.91 (s, 3H), 3.81-3.68 (m, 2H), 3.44 (t, J=6.9 Hz, 2H), 3.06 (s, 3H).

### Example 85—Synthesis of 6-[3-(5-Chloro-2-methoxypyridine-3-sulfonamido)-2,6-difluorophenyl]-N-(cyclopropylmethyl)-7-fluoro-1H-indazole-3-carboxamide

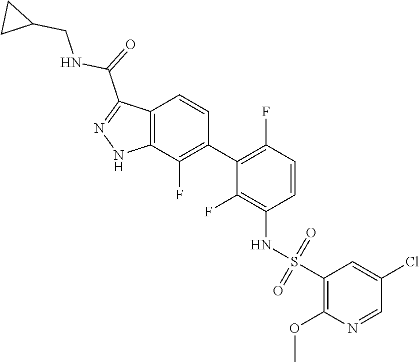 [SMILES: COc1ncc(Cl)cc1S(=O)(=O)Nc1ccc(F)c(-c2ccc3c(C(=O)NCC4CC4)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), 1-cyclopropylmethanamine (28 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, Xselect Fluoro-Phenyl, 19*150 mm, 5 um; mobile phase, Water (0.1% FA) and CH₃CN (40% CH₃CN up to 60% in 7 min); Detector, 254 nm & 220 nm. 6-[3-(5-Chloro-2-methoxypyridine-3-sulfonamido)-2,6-difluorophenyl]-N-(cyclopropylmethyl)-7-fluoro-1H-indazole-3-carboxamide (44 mg, 40% yield) was isolated as a white solid. LCMS (ES, m/z): [M+H]⁺: 566. ¹H NMR (300 MHz, DMSO-d₆): δ 14.33 (s, 1H), 10.45 (s, 1H), 8.60 (t, J=6.1 Hz, 1H), 8.51 (d, J=2.6 Hz, 1H), 8.30-7.88 (m, 2H), 7.46 (td, J=8.9, 5.9 Hz, 1H), 7.28 (td, J=9.1, 1.6 Hz, 1H), 7.22-7.04 (m, 1H), 3.91 (s, 3H), 3.20 (t, J=6.4 Hz, 2H), 1.18-0.91 (m, 1H), 0.60-0.41 (m, 2H), 0.29 (dt, J=4.8, 2.8 Hz, 2H).

### Example 86—Synthesis of 6-[3-(5-Chloro-2-methoxypyridine-3-sulfonamido)-2,6-difluorophenyl]-7-fluoro-N-[2-(morpholin-4-yl)ethyl]-1H-indazole-3-carboxamide

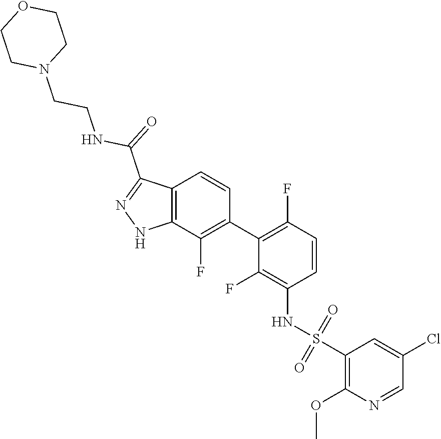 [SMILES: COc1ncc(Cl)cc1S(=O)(=O)Nc1ccc(F)c(-c2ccc3c(C(=O)NCCN4CCOCC4)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), N-aminoethylmorpholine (51 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, Xselect Fluoro-Phenyl, 19*150 mm, 5 um; mobile phase, Water (0.1% FA) and CH₃CN (15% CH₃CN up to 35% in 6 min); Detector, 254 nm & 220 nm. 6-[3-(5-Chloro-2-methoxypyridine-3-sulfonamido)-2,6-difluorophenyl]-7-fluoro-N-[2-(morpholin-4-yl)ethyl]-1H-indazole-3-carboxamide (61 mg, 50% yield) was isolated as a white solid. LCMS (ES, m/z): [M+H]⁺: 625. ¹H NMR (300 MHz, DMSO-d₆) δ 14.34 (s, 1H), 8.50 (d, J=2.6 Hz, 2H), 8.14 (s, 1H), 8.13-7.93 (m, 3H), 7.46 (d, J=5.9 Hz, 1H), 7.32-7.18 (m, 1H), 7.18-6.98 (m, 1H), 3.91 (s, 3H), 3.61 (t, J=4.7 Hz, 4H), 3.47 (q, J=6.5 Hz, 4H), 2.59 (d, J=6.9 Hz, 2H).

### Example 87—Synthesis of 6-[3-(5-Chloro-2-methoxypyridine-3-sulfonamido)-2,6-difluorophenyl]-7-fluoro-N-(2-hydroxycyclopentyl)-1H-indazole-3-carboxamide

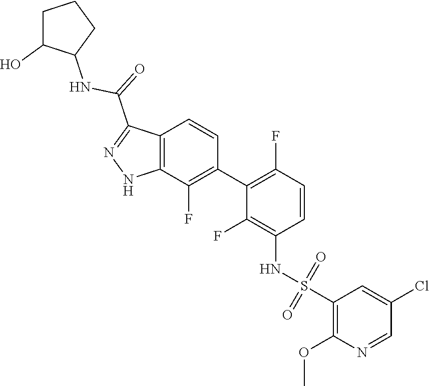 [SMILES: COc1ncc(Cl)cc1S(=O)(=O)Nc1ccc(F)c(-c2ccc3c(C(=O)NC4CCCC4O)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), 2-aminocyclopentan-1-ol (39 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, Xselect Fluoro-Phenyl, 19*150 mm, 5 um; mobile phase, Water (0.1% FA) and CH₃CN (40% CH₃CN up to 65% in 7 min); Detector, 254 nm & 220 nm. 6-[3-(5-Chloro-2-methoxypyridine-3-sulfonamido)-2,6-difluorophenyl]-7-fluoro-N-(2-hydroxycyclopentyl)-1H-indazole-3-carboxamide (26 mg, 23% yield) was isolated as a white solid. LCMS (ES, m/z): [M+H]⁺: 596. ¹H NMR (300 MHz, DMSO-d₆) δ 14.31 (s, 1H), 10.45 (s, 1H), 8.51 (d, J=2.6 Hz, 1H), 8.32 (d, J=7.0 Hz, 1H), 8.21-7.92 (m, 2H), 7.57-7.39 (m, 1H), 7.39-7.21 (m, 1H), 7.21-6.98 (m, 1H), 4.80 (d, J=4.2 Hz, 1H), 4.22-3.98 (m, 2H), 3.91 (s, 3H), 2.11-1.96 (m, 1H), 1.96-1.82 (m, 1H), 1.76-1.41 (m, 4H).

### Example 88—Synthesis of 6-[3-(5-Chloro-2-methoxypyridine-3-sulfonamido)-2,6-difluorophenyl]-7-fluoro-N-(2-hydroxycyclohexyl)-1H-indazole-3-carboxamide

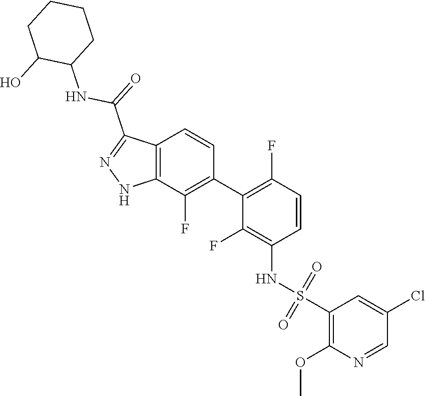 [SMILES: COc1ncc(Cl)cc1S(=O)(=O)Nc1ccc(F)c(-c2ccc3c(C(=O)NC4CCCCC4O)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), 2-aminocyclohexan-1-ol (45 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, Xselect Fluoro-Phenyl, 19*150 mm, 5 um; mobile phase, Water (0.1% FA) and CH₃CN (38% CH₃CN up to 55% in 7 min); Detector, 254 nm & 220 nm. 6-[3-(5-Chloro-2-methoxypyridine-3-sulfonamido)-2,6-difluorophenyl]-7-fluoro-N-(2-hydroxycyclohexyl)-1H-indazole-3-carboxamide (29 mg, 24% yield) was isolated as a white solid. LCMS (ES, m/z): [M+H]⁺: 610. ¹H NMR (300 MHz, DMSO-d₆) δ 14.30 (s, 1H), 10.45 (s, 1H), 8.51 (d, J=2.6 Hz, 1H), 8.17-7.96 (m, 3H), 7.56-7.37 (m, 1H), 7.37-7.22 (m, 1H), 7.22-7.05 (m, 1H), 4.66 (d, J=5.5 Hz, 1H), 3.91 (s, 3H), 3.78-3.59 (m, 1H), 3.59-3.42 (m, 1H), 1.99-1.86 (m, 2H), 1.74-1.58 (m, 2H), 1.42-1.21 (m, 4H).

### Example 89—Synthesis of 6-[3-(5-Chloro-2-methoxypyridine-3-sulfonamido)-2,6-difluorophenyl]-7-fluoro-N-(2-methylbut-3-yn-2-yl)-1H-indazole-3-carboxamide

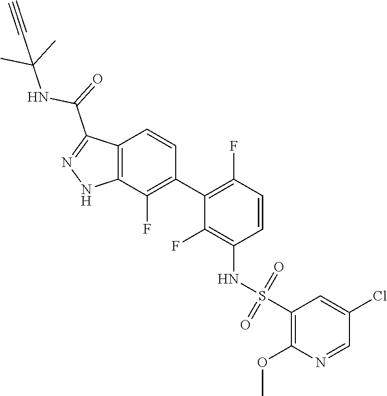 [SMILES: C#CC(C)(C)NC(=O)c1n[nH]c2c(F)c(-c3c(F)ccc(NS(=O)(=O)c4cc(Cl)cnc4OC)c3F)ccc12]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), 2-methylbut-3-yn-2-amine (32 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, Xselect Fluoro-Phenyl, 19*150 mm, 5 um; mobile phase, Water (0.1% FA) and CH₃CN (55% CH₃CN up to 75% in 7 min); Detector, 254 nm & 220 nm. 6-[3-(5-Chloro-2-methoxypyridine-3-sulfonamido)-2,6-difluorophenyl]-7-fluoro-N-(2-methylbut-3-yn-2-yl)-1H-indazole-3-carboxamide (40 mg, 35% yield) was isolated as a white solid. LCMS (ES, m/z): [M+H]⁺: 578. ¹H NMR (300 MHz, DMSO-d₆) δ 14.36 (s, 1H), 8.51 (d, J=2.6 Hz, 1H), 8.16-7.99 (m, 3H), 7.53-7.40 (m, 1H), 7.34-7.23 (m, 1H), 7.21-7.08 (m, 1H), 3.91 (s, 3H), 3.18 (s, 1H), 1.68 (s, 6H).

### Example 90—Synthesis of 6-[3-(5-Chloro-2-methoxypyridine-3-sulfonamido)-2,6-difluorophenyl]-7-fluoro-N-(2,2,2-trifluoroethyl)-1H-indazole-3-carboxamide

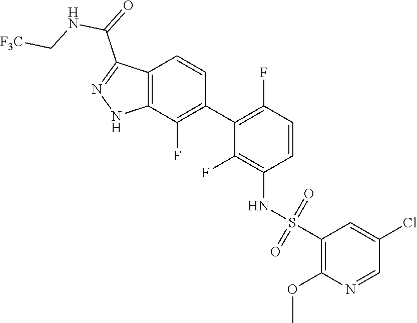 [SMILES: COc1ncc(Cl)cc1S(=O)(=O)Nc1ccc(F)c(-c2ccc3c(C(=O)NCC(F)(F)F)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), 2,2,2-trifluoroethylamine (39 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, Xselect Fluoro-Phenyl, 19*150 mm, 5 um; mobile phase, Water (0.1% FA) and CH₃CN (50% CH₃CN up to 73% in 7 min); Detector, 254 nm & 220 nm. 6-[3-(5-Chloro-2-methoxypyridine-3-sulfonamido)-2,6-difluorophenyl]-7-fluoro-N-(2,2,2-trifluoroethyl)-1H-indazole-3-carboxamide (48 mg, 42% yield) was isolated as a white solid. LCMS (ES, m/z): [M+H]⁺: 635. ¹H NMR (300 MHz, DMSO-d₆) δ 14.52 (s, 1H), 10.35 (s, 1H), 9.18 (t, J=6.6 Hz, 1H), 8.51 (d, J=2.6 Hz, 1H), 8.11-8.02 (m, 2H), 7.47 (td, J=8.9, 5.9 Hz, 1H), 7.29 (t, J=8.9 Hz, 1H), 7.21-7.12 (m, 1H), 4.11 (dd, J=9.7, 6.7 Hz, 2H), 3.91 (s, 3H).

### Example 91—Synthesis of 6-[3-(5-Chloro-2-methoxypyridine-3-sulfonamido)-2,6-difluorophenyl]-N-[cyano(cyclopropyl)methyl]-7-fluoro-1H-indazole-3-carboxamide

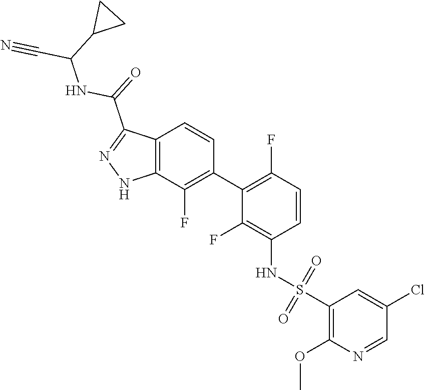 [SMILES: COc1ncc(Cl)cc1S(=O)(=O)Nc1ccc(F)c(-c2ccc3c(C(=O)NC(C#N)C4CC4)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), 2-amino-2-cyclopropylacetonitrile (38 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, Xselect Fluoro-Phenyl, 19*150 mm, 5 um; mobile phase, Water (0.1% FA) and CH₃CN (48% CH₃CN up to 70% in 7 min); Detector, 254 nm & 220 nm. 6-[3-(5-Chloro-2-methoxypyridine-3-sulfonamido)-2,6-difluorophenyl]-N-[cyano(cyclopropyl)methyl]-7-fluoro-1H-indazole-3-carboxamid (20 mg, 17% yield) was isolated as a white solid. LCMS (ES, m/z): [M+H]⁺: 591. ¹H NMR (300 MHz, DMSO-d₆): δ 9.55 (d, J=8.1 Hz, 1H), 8.42 (d, J=2.7 Hz, 1H), 8.09-7.83 (m, 2H), 7.40 (d, J=6.7 Hz, 1H), 7.19 (dd, J=8.3, 5.6 Hz, 2H), 4.67-4.20 (m, 1H), 3.87 (s, 3H), 1.86-1.34 (m, 1H), 0.76-0.57 (m, 3H), 0.44 (q, J=5.1 Hz, 1H).

### Example 92—Synthesis of 6-[3-(5-Chloro-2-methoxypyridine-3-sulfonamido)-2,6-difluorophenyl]-7-fluoro-N-(prop-2-yn-1-yl)-1H-indazole-3-carboxamide

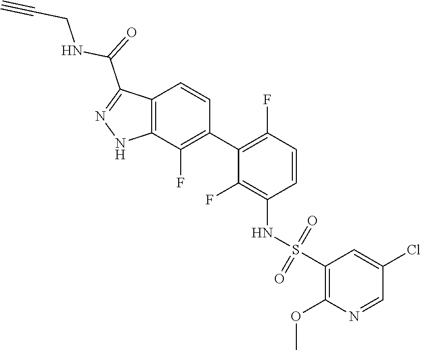 [SMILES: C#CCNC(=O)c1n[nH]c2c(F)c(-c3c(F)ccc(NS(=O)(=O)c4cc(Cl)cnc4OC)c3F)ccc12]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), 2-propynylamine (22 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, T3, 19*150 mm, Sum; mobile phase, Water (0.1% FA) and CH₃CN (50% CH₃CN up to 70% in 6 min); Detector, 254 nm & 220 nm. 6-[3-(5-Chloro-2-methoxypyridine-3-sulfonamido)-2,6-difluorophenyl]-7-fluoro-N-(prop-2-yn-1-yl)-1H-indazole-3-carboxamide (15 mg, 14% yield) was isolated as a white solid. LCMS (ES, m/z): [M+H]⁺: 550. ¹H NMR (300 MHz, DMSO-d₆) δ 14.42 (s, 1H), 10.44 (s, 1H), 9.07-8.94 (m, 1H), 8.50 (d, J=2.6 Hz, 1H), 8.13-7.98 (m, 2H), 7.52-7.40 (m, 1H), 7.35-7.23 (m, 1H), 7.23-7.10 (m, 1H), 4.16-4.03 (m, 2H), 3.90 (s, 3H), 3.15-3.05 (m, 1H).

### Example 93—Synthesis of 6-[3-(5-Chloro-2-methoxypyridine-3-sulfonamido)-2,6-difluorophenyl]-7-fluoro-N-(piperidin-3-ylmethyl)-1H-indazole-3-carboxamide

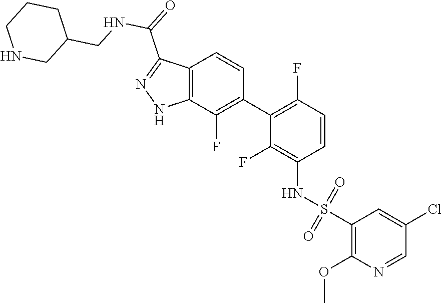 [SMILES: COc1ncc(Cl)cc1S(=O)(=O)Nc1ccc(F)c(-c2ccc3c(C(=O)NCC4CCCNC4)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (150 mg, 0.3 mmol, 1 equiv), CH₃CN (3 mL), tert-butyl 3-(aminomethyl)piperidine-1-carboxylate (125 mg, 0.6 mmol, 1.5 equiv), NMI (84 mg, 1 mmol, 3.5 equiv), TCFH (123 mg, 0.4 mmol, 1.5 equiv), TFA (2 mL) and DCM (2 mL). The resulting solution was stirred overnight at room temperature. The resulting mixture was concentrated under vacuum. The residue was diluted in DCM (2 mL). TFA (2 mL) was added to the mixture. The resulting reaction mixture was stirred for 1 h at room temperature. The resulting mixture was concentrated under vacuum. The residue was diluted in MeOH (4 mL). The filtrate was purified by Prep-HPLC with the following conditions: Column, T3, 19*150 mm, Sum; mobile phase, Water (0.1% FA) and CH₃CN (23% CH₃CN up to 45% in 7 min); Detector, 254 nm & 220 nm. 6-[3-(5-Chloro-2-methoxypyridine-3-sulfonamido)-2,6-difluorophenyl]-7-fluoro-N-(piperidin-3-ylmethyl)-1H-indazole-3-carboxamide (69 mg, 39% yield) was isolated as a white solid. LCMS (ES, m/z): [M+H]⁺: 609. ¹H NMR (300 MHz, DMSO-d₆) δ 8.84-8.71 (m, 1H), 8.23 (d, J=2.7 Hz, 1H), 8.08-7.94 (m, 2H), 7.31-7.11 (m, 2H), 7.01-6.85 (m, 1H), 3.80 (s, 3H), 3.26-3.22 (m, 2H), 3.21-3.10 (m, 2H), 2.89-2.71 (m, 1H), 2.71-2.57 (m, 1H), 2.14-1.95 (m, 1H), 1.89-1.71 (m, 2H), 1.69-1.48 (m, 1H), 1.34-1.19 (m, 1H).

### Example 94—Synthesis of N-(2-Aminocyclohexyl)-6-[3-(5-chloro-2-methoxypyridine-3-sulfonamido)-2,6-difluorophenyl]-7-fluoro-1H-indazole-3-carboxamide

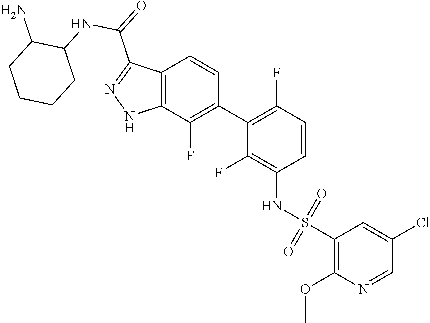 [SMILES: COc1ncc(Cl)cc1S(=O)(=O)Nc1ccc(F)c(-c2ccc3c(C(=O)NC4CCCCC4N)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (150 mg, 0.3 mmol, 1 equiv), CH₃CN (3 mL), tert-butyl N-(2-aminocyclohexyl)carbamate (125 mg, 0.6 mmol, 2 equiv), NMI (84 mg, 1 mmol, 3.5 equiv), TCFH (123 mg, 0.4 mmol, 1.5 equiv). The resulting solution was stirred overnight at room temperature. The resulting mixture was concentrated under vacuum. The residue was diluted in DCM (2 mL). TFA (2 mL) was added to the mixture. The resulting reaction mixture was stirred for 1 h at room temperature. The resulting mixture was concentrated under vacuum. The residue was diluted in MeOH (4 mL). The filtrate was purified by Prep-HPLC with the following conditions: Column, T3, 19*150 mm, Sum; mobile phase, Water (0.1% FA) and CH₃CN (25% CH₃CN up to 45% in 7 min); Detector, 254 nm & 220 nm. N-(2-Aminocyclohexyl)-6-[3-(5-chloro-2-methoxypyridine-3-sulfonamido)-2,6-difluorophenyl]-7-fluoro-1H-indazole-3-carboxamide (48 mg, 27% yield) was isolated as a white solid. LCMS (ES, m/z): [M+H]⁺: 609. ¹H NMR (300 MHz, DMSO-d₆) δ 8.34-8.18 (m, 2H), 8.08-7.88 (m, 2H), 7.42-7.08 (m, 2H), 7.00-6.87 (m, 1H), 4.41-4.20 (m, 1H), 4.09-3.90 (m, 1H), 3.81 (s, 3H), 3.50-3.33 (m, 1H), 3.25-3.09 (m, 1H), 2.18-1.04 (m, 8H).

### Example 95—Synthesis of N-(1-Aminopropan-2-yl)-6-[3-(5-chloro-2-methoxypyridine-3-sulfonamido)-2,6-difluorophenyl]-7-fluoro-1H-indazole-3-carboxamide

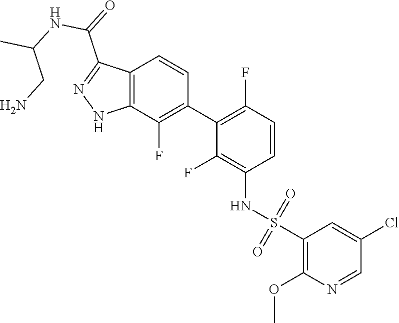 [SMILES: COc1ncc(Cl)cc1S(=O)(=O)Nc1ccc(F)c(-c2ccc3c(C(=O)NC(C)CN)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (150 mg, 0.3 mmol, 1 equiv), CH₃CN (3 mL), tert-butyl N-(2-aminopropyl)carbamate (102 mg, 0.6 mmol, 2 equiv), NMI (84 mg, 1 mmol, 3.5 equiv), TCFH (123 mg, 0.4 mmol, 1.5 equiv). The resulting solution was stirred overnight at room temperature. The resulting mixture was concentrated under vacuum. The residue was diluted in DCM (2 mL). TFA (2 mL) was added to the mixture. The resulting reaction mixture was stirred for 1 h at room temperature. The resulting mixture was concentrated under vacuum. The residue was diluted in MeOH (4 mL). The filtrate was purified by Prep-HPLC with the following conditions: Column, T3, 19*150 mm, Sum; mobile phase, Water (0.1% FA) and CH₃CN (28% CH₃CN up to 40% in 7 min); Detector, 254 nm & 220 nm. N-(1-Aminopropan-2-yl)-6-[3-(5-chloro-2-methoxypyridine-3-sulfonamido)-2,6-difluorophenyl]-7-fluoro-1H-indazole-3-carboxamide (71 mg, 43% yield) was isolated as a white solid. LCMS (ES, m/z): [M+H]⁺: 569. ¹H NMR (300 MHz, DMSO-d₆) δ 8.49 (d, J=8.7 Hz, 1H), 8.21 (d, J=2.7 Hz, 1H), 8.07-7.93 (m, 2H), 7.34-7.11 (m, 2H), 6.97-6.80 (m, 1H), 4.46-4.26 (m, 1H), 3.80 (s, 3H), 3.08-2.93 (m, 2H), 1.39 (s, 1H), 1.24 (d, J=6.7 Hz, 3H).

### Example 96—Synthesis of 6-[3-(5-Chloro-2-methoxypyridine-3-sulfonamido)-2,6-difluorophenyl]-7-fluoro-N-(4-oxocyclohexyl)-1H-indazole-3-carboxamide

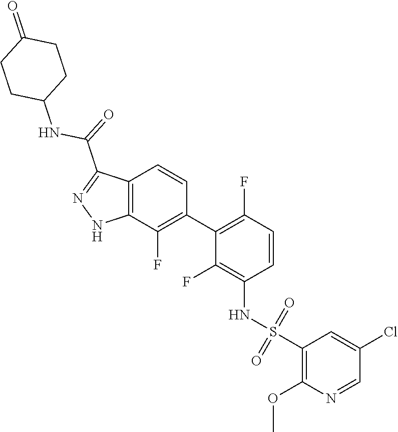 [SMILES: COc1ncc(Cl)cc1S(=O)(=O)Nc1ccc(F)c(-c2ccc3c(C(=O)NC4CCC(=O)CC4)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), 4-aminocyclohexan-1-one (44 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, Welch-Xtimate, 30*150 mm, 10 um; mobile phase, Water (0.1% FA) and CH₃CN (38% CH₃CN up to 78% in 6 min); Detector, 254 nm & 220 nm. 6-[3-(5-Chloro-2-methoxypyridine-3-sulfonamido)-2,6-difluorophenyl]-7-fluoro-N-(4-oxocyclohexyl)-1H-indazole-3-carboxamide (10 mg, 8% yield) was isolated as a white solid. LCMS (ES, m/z): [M+H]⁺: 608. ¹H NMR (300 MHz, DMSO-d₆) δ 14.35 (s, 1H), 10.45 (s, 1H), 8.62-8.41 (m, 2H), 8.21-7.89 (m, 2H), 7.52-7.40 (m, 1H), 7.34-7.22 (m, 1H), 7.20-7.10 (m, 1H), 4.53-4.30 (m, 1H), 3.91 (s, 3H), 2.66-2.55 (m, 2H), 2.37-2.22 (m, 2H), 2.19-2.04 (m, 2H), 2.02-1.80 (m, 2H).

### Example 97—Synthesis of 6-[3-(5-Chloro-2-methoxypyridine-3-sulfonamido)-2,6-difluorophenyl]-7-fluoro-N-(3-hydroxycyclopentyl)-1H-indazole-3-carboxamide

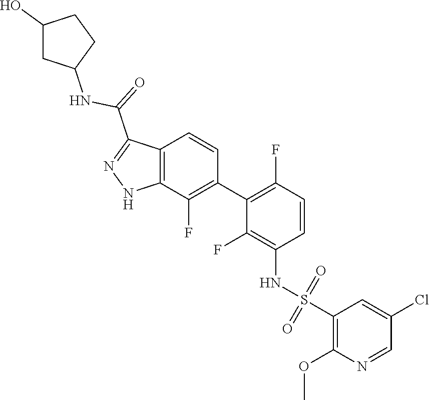 [SMILES: COc1ncc(Cl)cc1S(=O)(=O)Nc1ccc(F)c(-c2ccc3c(C(=O)NC4CCC(O)C4)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), 3-aminocyclopentan-1-ol (40 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, T3, 19*150 mm, 5 um; mobile phase, Water (0.1% FA) and CH₃CN (40% CH₃CN up to 75% in 6 min); Detector, 254 nm & 220 nm. 6-[3-(5-Chloro-2-methoxypyridine-3-sulfonamido)-2,6-difluorophenyl]-7-fluoro-N-(3-hydroxycyclopentyl)-1H-indazole-3-carboxamide (33 mg, 28% yield) was isolated as a white solid. LCMS (ES, m/z): [M+H]⁺: 596. ¹H NMR (300 MHz, DMSO-d₆) δ 14.30 (s, 1H), 10.46 (s, 1H), 8.49 (d, J=2.5 Hz, 1H), 8.33 (d, J=8.4 Hz, 1H), 8.11-8.00 (m, 2H), 7.52-7.39 (m, 1H), 7.33-7.21 (m, 1H), 7.19-7.07 (m, 1H), 4.82 (d, J=3.8 Hz, 1H), 4.56-4.33 (m, 1H), 4.33-4.04 (m, 1H), 3.90 (s, 3H), 2.15-1.88 (m, 2H), 1.88-1.54 (m, 4H).

### Example 98—Synthesis of N-(3-Aminocyclohexyl)-6-[3-(5-chloro-2-methoxypyridine-3-sulfonamido)-2,6-difluorophenyl]-7-fluoro-1H-indazole-3-carboxamide

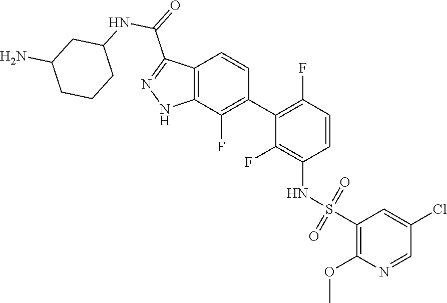 [SMILES: COc1ncc(Cl)cc1S(=O)(=O)Nc1ccc(F)c(-c2ccc3c(C(=O)NC4CCCC(N)C4)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (150 mg, 0.3 mmol, 1 equiv), CH₃CN (3 mL), tert-butyl N-(3-aminocyclohexyl)carbamate (125 mg, 0.6 mmol, 2 equiv), NMI (84 mg, 1 mmol, 3.5 equiv), TCFH (123 mg, 0.44 mmol, 1.5 equiv). The resulting solution was stirred overnight at room temperature. The reaction was concentrated under vacuum. The residue was diluted with TFA (1.5 mL) and DCM (1.5 mL). The resulting solution was stirred for 1 h at room temperature. The reaction was concentrated under vacuum. The residue was diluted with MeOH (3 mL). The reaction mixture was purified by Prep-HPLC with the following conditions: Column, T3, 19*150 mm, Sum; mobile phase, Water (0.1% FA) and CH₃CN (25% CH₃CN up to 45% in 7 min); Detector, 254 nm & 220 nm. N-(3-Aminocyclohexyl)-6-[3-(5-chloro-2-methoxypyridine-3-sulfonamido)-2,6-difluorophenyl]-7-fluoro-1H-indazole-3-carboxamide (34.8 mg, 20% yield) was isolated as a white solid. LCMS (ES, m/z): [M+H]⁺: 609. ¹H NMR (300 MHz, DMSO-d₆) δ 8.61-8.17 (m, 3H), 8.07-7.94 (m, 2H), 7.36-7.12 (m, 2H), 7.02-6.87 (m, 1H), 4.43-4.27 (m, 1H), 3.81 (s, 3H), 3.57-3.49 (m, 1H), 2.24-1.96 (m, 1H), 1.96-1.15 (m, 7H).

### Example 99—Synthesis of 6-[3-(5-Chloro-2-methoxypyridine-3-sulfonamido)-2,6-difluorophenyl]-7-fluoro-N-[2-(2-oxopyrrolidin-1-yl)ethyl]-1H-indazole-3-carboxamide

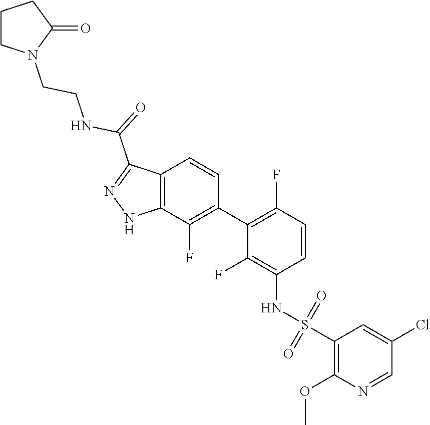 [SMILES: COc1ncc(Cl)cc1S(=O)(=O)Nc1ccc(F)c(-c2ccc3c(C(=O)NCCN4CCCC4=O)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), 1-(2-aminoethyl)pyrrolidin-2-one (50 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, T3, 19*150 mm, 5 um; mobile phase, Water (0.1% FA) and CH₃CN (25% CH₃CN up to 75% in 7 min); Detector, 254 nm & 220 nm. 6-[3-(5-Chloro-2-methoxypyridine-3-sulfonamido)-2,6-difluorophenyl]-7-fluoro-N-[2-(2-oxopyrrolidin-1-yl)ethyl]-1H-indazole-3-carboxamide (18 mg, 15% yield) was isolated as a white solid. LCMS (ES, m/z): [M+H]: 623. ¹H NMR (300 MHz, DMSO-d₆) δ 14.38 (s, 1H), 10.46 (s, 1H), 8.64 (s, 1H), 8.43 (s, 1H), 8.04 (s, 2H), 7.59-7.30 (m, 1H), 7.28 (s, 1H), 7.14 (s, 1H), 3.97 (s, 3H), 3.47 (s, 5H), 2.21 (s, 2H), 1.89 (s, 2H).

### Example 100—Synthesis of 6-[3-(5-Chloro-2-methoxypyridine-3-sulfonamido)-2,6-difluorophenyl]-7-fluoro-N-[(1-methylpyrrolidin-3-yl)methyl]-1H-indazole-3-carboxamide

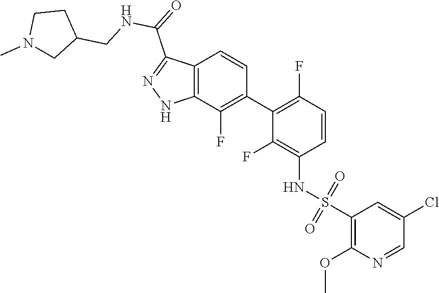 [SMILES: COc1ncc(Cl)cc1S(=O)(=O)Nc1ccc(F)c(-c2ccc3c(C(=O)NCC4CCN(C)C4)n[nH]c3c2F)c1F]

Into a 40-mL vial, was placed 6-[3-(5-chloro-2-methoxypyridine-3-sulfonamido)-2,6-difluorophenyl]-7-fluoro-1H-indazole-3-carboxylic acid (100 mg, 0.2 mmol, 1 equiv), CH₃CN (3 mL), 1-(1-methylpyrrolidin-3-yl)methanamine (45 mg, 0.4 mmol, 2 equiv), NMI (56 mg, 0.7 mmol, 3.5 equiv) and TCFH (82 mg, 0.3 mmol, 1.5 equiv). The resulting solution was stirred overnight at room temperature, then quenched by the addition of 1 mL of water. The resultant mixture was purified by Prep-HPLC with the following conditions: Column, Welch-Xtimate, 30*150 mm, 10 um; mobile phase, Water (0.1% FA) and CH3CN (15% CH3CN up to 60% in 6 min); Detector, 254 nm & 220 nm. 6-[3-(5-Chloro-2-methoxypyridine-3-sulfonamido)-2,6-difluorophenyl]-7-fluoro-N-[(1-methylpyrrolidin-3-yl)methyl]-1H-indazole-3-carboxamide (40 mg, 33% yield) was isolated as a white solid. LCMS (ES, m/z): [M+H]⁺: 609. ¹H NMR (300 MHz, DMSO-d₆) δ 8.87-8.68 (m, 1H), 8.34 (d, J=2.6 Hz, 1H), 8.23 (s, 1H), 8.11-7.96 (m, 2H), 7.42-7.27 (m, 1H), 7.22-7.12 (m, 1H), 7.12-6.99 (m, 1H), 3.84 (s, 3H), 3.44-3.31 (m, 2H), 3.19-2.91 (m, 3H), 2.91-2.75 (m, 1H), 2.72-2.56 (m, 4H), 2.12-1.90 (m, 1H), 1.81-1.59 (m, 1H).

### Example 101—Synthesis of 6-[3-(1,3-dihydro-2-benzofuran-4-sulfonamido)-2,6-difluorophenyl]-7-fluoro-N-methyl-1H-indazole-3-carboxamide

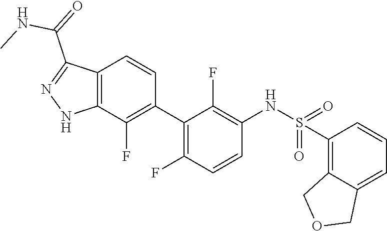 [SMILES: CNC(=O)c1n[nH]c2c(F)c(-c3c(F)ccc(NS(=O)(=O)c4cccc5c4COC5)c3F)ccc12]

### Part I—Synthesis of 1,2-bis bromomethyl)-3-nitrobenzene

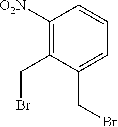 [SMILES: O=[N+]([O-])c1cccc(CBr)c1CBr]

To a stirred solution of 1,2-dimethyl-3-nitrobenzene (20 g, 132 mmol, 1 equiv) in CCl₄ (83.00 mL) was added NBS (49.5 g, 278 mmol, 2.1 equiv) and BPO (339 mg, 1.3 mmol, 0.01 equiv) at room temperature. The reaction was stirred at 80° C. for 2 hours and then cooled to room temperature. Further BPO (1.36 g, 5.3 mmol, 0.04 equiv) was added and the resulting solution was stirred for another 2 hours at 80° C. The mixture was allowed to cool, and the solids removed by filtration, being washed with CCl₄ (100 mL). The filtrate was concentrated under reduced pressure to give 1,2-bis bromomethyl)-3-nitrobenzene (44 g, crude) as a yellow oil. The material was used for next step directly without further purification.

### Part 11—Synthesis of 4-nitro-1,3-dihydro-2-benzofuran

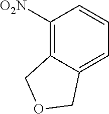 [SMILES: O=[N+]([O-])c1cccc2c1COC2]

To a stirred solution of 1,2-bis(bromomethyl)-3-nitrobenzene (44 g, 0.14 mol, 1 equiv) in toluene (440 mL) and H₂O (13.2 mL) was added aluminum oxide (290 g, 2.9 mol, 20 equiv), and the reaction was stirred at 120° C. overnight. The mixture was allowed to cool and the resulting mixture was filtered. The filtrate was concentrated under reduced pressure and the residue was purified by silica gel column chromatography, eluting with PE/EA (20:1) to afford 4-nitro-1,3-dihydro-2-benzofuran (8.0 g, 34%) as a red solid. LCMS (ES, m/z): [M+H]⁺: 166.

### Part III—Synthesis of 1,3-dihydro-2-benzofuran-4-amine

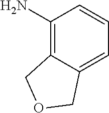

To a stirred solution of 4-nitro-1,3-dihydro-2-benzofuran (5 g, 30 mmol, 1 equiv) in THE (80 mL) was added Raney Ni (5 g, 58 mmol, 1.9 equiv) at room temperature under N2 atmosphere. The vessel was purged with hydrogen 3 times, and the resulting suspension was stirred at room temperature under hydrogen (5 atm) for 2 hours. The solids were carefully removed by filtration, washing with THE (50 mL). The filtrate was concentrated under reduced pressure to give 1,3-dihydro-2-benzofuran-4-amine (2.5 g, 61%) as a yellow solid. LCMS (ES, m/z): [M+H]⁺: 136.

### Part IV—Synthesis of 4-bromo-1,3-dihydro-2-benzofuran

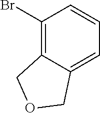

1,3-dihydro-2-benzofuran-4-amine (2.5 g, 18.5 mmol, 1 equiv) was dissolved in ACN (25 mL) and cooled to 0° C. HBr in water (7.5 mL, 257 mmol, 14 equiv) was added dropwise, and the reaction was stirred for 10 min, the solution turning to a thick suspension. NaNO₂ (1.4 g, 20 mmol, 1.1 equiv) was added and stirred for another 10 min, the reaction mixture turning clear again. CuBr (6.63 g, 46 mmol, 2.5 equiv) was added over 10 mins, and the resulting solution was stirred at 0° C. for 1 hour. The mixture was diluted with H₂O (20 mL) and extracted with Et₂O (30 mL×3). The combined organic layers were washed with brine (10 mL), dried over anhydrous Na₂SO₄, then after filtration, the filtrate was concentrated under reduced pressure. The residue was purified by silica gel column chromatography, eluting with PE to afford 4-bromo-1,3-dihydro-2-benzofuran (3.0 g, 81%) as a yellow solid. LCMS (ES, m/z): [M+H]⁺: 199.

### Part V—Synthesis of 4-(benzylsulfanyl)-1,3-dihydro-2-benzofuran

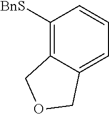

To a stirred solution of 4-bromo-1,3-dihydro-2-benzofuran (3.0 g, 15 mmol, 1 equiv) in dioxane (37.5 mL) was added benzyl mercaptan (2.2 g, 18 mmol, 1.2 eq), Pd₂(dba)₃.CHCl₃ (1.6 g, 1.5 mmol, 0.1 equiv), XantPhos (1.74 g, 3 mmol, 0.2 equiv) and DIEA (3.9 g, 30 mmol, 2 equiv). The solution was purged with N₂ and stirred at 120° C. overnight. The mixture was cooled, filtered, and the filtrate was concentrated under reduced pressure. The residue was purified by silica gel column chromatography, eluting with PE/EA (20:1) to afford 4-(benzylsulfanyl)-1,3-dihydro-2-benzofuran (3.0 g, 82% yield) as a yellow oil. LCMS (ES, m/z): [M+H]⁺: 243.

### Part VI—Synthesis of 1,3-dihydroisobenzofuran-4-sulfonyl chloride

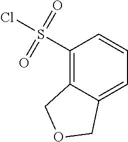 [SMILES: O=S(=O)(Cl)c1cccc2c1COC2]

To a solution of 4-(benzylsulfanyl)-1,3-dihydro-2-benzofuran (1.2 g, 5 mmol, 1 equiv) in MeCN (24 mL) was added HCl (6M, 12 mL) at 0° C., then NCS (2.65 g, 19.8 mmol, 4 equiv) was added slowly at 0° C. The reaction was stirred at 0° C. for 10 min, then diluted with 12 ml of H₂O, the mixture was extracted with EA, 10 mL×3, and the combined organic layers washed with brine (10 mL), and dried over anhydrous Na2SO4 before being concentrated under reduced pressure to afford 1,3-dihydroisobenzofuran-4-sulfonyl chloride (0.50 g, crude) as a yellow oil.

### Part VII—Synthesis of 6-[3-(1,3-dihydro-2-benzofuran-4-sulfonamido)-2,6-difluorophenyl]-7-fluoro-N-methyl-1-[[2-(trimethylsilyl)ethoxy]methyl]indazole-3-carboxamide

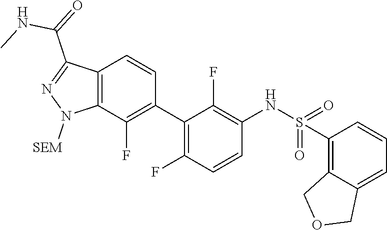 [SMILES: CNC(=O)c1nn(COCC[Si](C)(C)C)c2c(F)c(-c3c(F)ccc(NS(=O)(=O)c4cccc5c4COC5)c3F)ccc12]

6-(3-amino-2,6-difluorophenyl)-7-fluoro-N-methyl-1-((2-(trimethylsilyl)ethoxy)methyl)-1H-indazole-3-carboxamide (See Example 45) (0.20 g, 0.44 mmol, 1 equiv) was dissolved in DCM (2 mL) and cooled to 0° C., then pyridine (0.17 g, 2.2 mmol, 5 equiv) and 1,3-dihydroisobenzofuran-4-sulfonyl chloride (0.11 g, 0.48 mmol, 1.1 equiv) were added. The reaction was stirred at 0° C. for 10 min. The resulting solution was diluted with DCM (10 mL) and washed with H₂O (3 mL×3). The organic layer was dried over anhydrous Na₂SO₄ and concentrated under reduced pressure to afford 6-[3-(1,3-dihydro-2-benzofuran-4-sulfonamido)-2,6-difluorophenyl]-7-fluoro-N-methyl-1-[[2-(trimethylsilyl)ethoxy]methyl]indazole-3-carboxamide (0.10 g, crude) as a yellow solid. LCMS (ES, m/z): [M+H]⁺: 633.

### Part VIII—Synthesis of 6-[3-(1,3-dihydro-2-benzofuran-4-sulfonamido)-2,6-difluorophenyl]-7-fluoro-N-methyl-1H-indazole-3-carboxamide

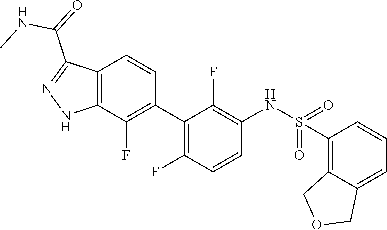 [SMILES: CNC(=O)c1n[nH]c2c(F)c(-c3c(F)ccc(NS(=O)(=O)c4cccc5c4COC5)c3F)ccc12]

To a stirred solution of 6-[3-(1,3-dihydro-2-benzofuran-4-sulfonamido)-2,6-difluorophenyl]-7-fluoro-N-methyl-1-[[2-(trimethylsilyl)ethoxy]methyl]indazole-3-carboxamide (100 mg, 0.16 mmol, 1 equiv) in DCM (2 mL) was added TFA (54 mg, 0.48 mmol, 3 equiv) at 0° C. and the reaction was stirred at room temperature for 2 hours. The solution was concentrated and the crude product was purified by Flash-Prep-HPLC with the following conditions (IntelFlash-1): Column, C18 silica gel; mobile phase, Mobile Phase A: Water (0.05% FA), Mobile Phase B: ACN=Gradient: 40 B to 70 B; Detector, 220 to afford 6-[3-(1,3-dihydro-2-benzofuran-4-sulfonamido)-2,6-difluorophenyl]-7-fluoro-N-methyl-1H-indazole-3-carboxamide (43 mg, 55%) as a white solid. LCMS (ES, m/z): [M+H]⁺: 503. ¹H NMR (300 MHz, DMSO-d6) δ 14.30 (s, 1H), 10.33 (s, 1H), 8.51 (d, J=4.8 Hz, 1H), 8.04 (d, J=8.4 Hz, 1H), 7.62-7.26 (m, 5H), 7.06-7.01 (m, 1H), 5.04 (s, 4H), 2.84 (d, J=4.7 Hz, 3H).

### Example 102—Synthesis of 6-[3-(2,3-dihydro-1-benzofuran-6-sulfonamido)-2,6-difluorophenyl]-7-fluoro-N-methyl-1H-indazole-3-carboxamide

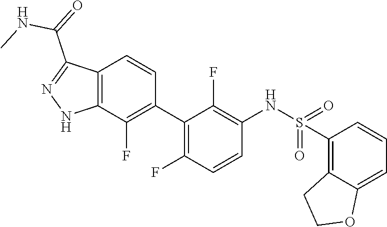 [SMILES: CNC(=O)c1n[nH]c2c(F)c(-c3c(F)ccc(NS(=O)(=O)c4cccc5c4CCO5)c3F)ccc12]

### Part I—Synthesis of 6-[3-(2,3-dihydro-1-benzofuran-4-sulfonamido)-2,6-difluorophenyl]-7-fluoro-N-methyl-1H-indazole-3-carboxamide

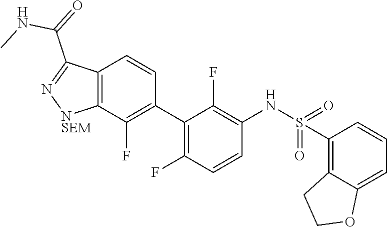 [SMILES: CNC(=O)c1nn(COCC[Si](C)(C)C)c2c(F)c(-c3c(F)ccc(NS(=O)(=O)c4cccc5c4CCO5)c3F)ccc12]

Into a 50-mL pressure tank reactor purged and maintained with an inert atmosphere of nitrogen, was placed 6-[3-(1-benzofuran-4-sulfonamido)-2,6-difluorophenyl]-7-fluoro-N-methyl-1-[[2-(trimethylsilyl)ethoxy]methyl]indazole-3-carboxamide (200 mg, 0.32 mmol, 1 equiv), MeOH (5 mL) and 10% Pd/C (30 mg). The mixture was stirred for 16 h at room temperature under an atmosphere of hydrogen. The solids were removed by filtration and the filtrate concentrated to give 6-[3-(2,3-dihydro-1-benzofuran-4-sulfonamido)-2,6-difluorophenyl]-7-fluoro-N-methyl-1H-indazole-3-carboxamide (180 mg, 90% yield) as a white solid. LCMS (ES, m/z): [M+H]⁺: 633.

### Part II—Synthesis of 6-[3-(2,3-dihydro-1-benzofuran-6-sulfonamido)-2,6-difluorophenyl]-7-fluoro-N-methyl-1H-indazole-3-carboxamide

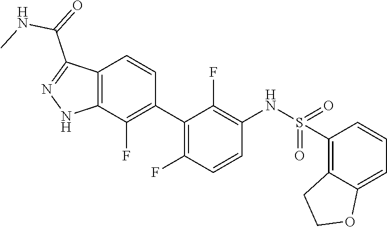 [SMILES: CNC(=O)c1n[nH]c2c(F)c(-c3c(F)ccc(NS(=O)(=O)c4cccc5c4CCO5)c3F)ccc12]

Into a 50-mL round-bottom flask, was placed 6-[3-(2,3-dihydro-1-benzofuran-6-sulfonamido)-2,6-difluorophenyl]-7-fluoro-N-methyl-1-[[2-(trimethylsilyl)ethoxy]methyl]indazole-3-carboxamide (100 mg, 0.16 mmol, 1 equiv) in DCM (5 mL). This was followed by the addition of TFA (1 mL) dropwise with stirring at 0 degrees C. The solution was stirred for 6 hr at room temperature, then diluted with 5 mL of water. The pH was adjusted to 8 with NaHCO₃ (2 mol/L) and the resulting solution was extracted with 2×10 mL of ethyl acetate. The extracts were dried (Na2SO4) and concentrated under vacuum. The crude product was purified by Prep-HPLC with the following conditions: Water (0.1% FA) and ACN (71% ACN up to 86% in 7 min, hold 95% in 1 min, down to 71% in 1 min, hold 71% in 1 min) to yield 6-[3-(2,3-dihydro-1-benzofuran-6-sulfonamido)-2,6-difluorophenyl]-7-fluoro-N-methyl-1H-indazole-3-carboxamide (21 mg, 26% yield) as a white solid. LCMS (ES, m/z): [M+H]⁺: 503. ¹H NMR (300 MHz, DMSO-d₆) δ 14.31 (s, 1H), 10.29 (s, 1H), 8.51 (d, J=4.8 Hz, 1H), 8.04 (d, J=8.3 Hz, 1H), 7.39 (td, J=8.8, 5.9 Hz, 1H), 7.31-7.23 (m, 2H), 7.14-6.97 (m, 3H), 4.55 (t, J=8.8 Hz, 2H), 3.30 (s, 2H), 2.84 (d, J=4.6 Hz, 3H).

### Example 103—Synthesis of 6-[3-(2,3-Dihydro-1-benzofuran-6-sulfonamido)-2,6-difluorophenyl]-7-fluoro-N-methyl-1-[[2-(trimethylsilyl)ethoxy]methyl]indazole-3-carboxamide

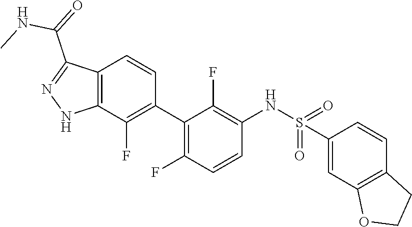 [SMILES: CNC(=O)c1n[nH]c2c(F)c(-c3c(F)ccc(NS(=O)(=O)c4ccc5c(c4)OCC5)c3F)ccc12]

### Part I—Synthesis of 6-[3-(2,3-Dihydro-1-benzofuran-6-sulfonamido)-2,6-difluorophenyl]-7-fluoro-N-methyl-1-[[2-(trimethylsilyl)ethoxy]methyl]indazole-3-carboxamide

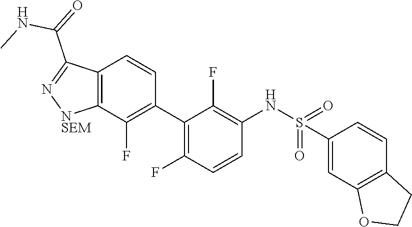 [SMILES: CNC(=O)c1nn(COCC[Si](C)(C)C)c2c(F)c(-c3c(F)ccc(NS(=O)(=O)c4ccc5c(c4)OCC5)c3F)ccc12]

Into a 50-mL pressure tank reactor purged and maintained with an inert atmosphere of nitrogen, was placed 6-[3-(1-benzofuran-6-sulfonamido)-2,6-difluorophenyl]-7-fluoro-N-methyl-1-[[2-(trimethylsilyl)ethoxy]methyl]indazole-3-carboxamide (See Example 61) (200 mg, 0.32 mmol, 1 equiv), MeOH (5 mL) and 10% Pd/C (30 mg). The mixture was stirred for 16 h at room temperature under an atmosphere of hydrogen. The solids were removed by filtration and the filtrate concentrated under vacuum. 6-[3-(2,3-Dihydro-1-benzofuran-6-sulfonamido)-2,6-difluorophenyl]-7-fluoro-N-methyl-1-[[2-(trimethylsilyl)ethoxy]methyl]indazole-3-carboxamide (160 mg, 80% yield) was isolated as a white solid. LCMS (ES, m/z): [M+H]⁺: 633.

### Part II—Synthesis of 6-[3-(2,3-Dihydro-1-benzofuran-6-sulfonamido)-2,6-difluorophenyl]-7-fluoro-N-methyl-1-[[2-(trimethylsilyl)ethoxy]methyl]indazole-3-carboxamide

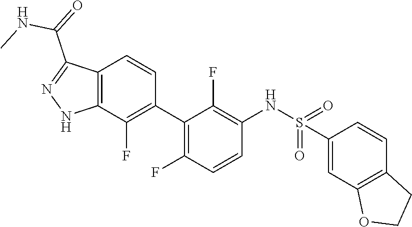 [SMILES: CNC(=O)c1n[nH]c2c(F)c(-c3c(F)ccc(NS(=O)(=O)c4ccc5c(c4)OCC5)c3F)ccc12]

Into a 8-mL round-bottom flask, was placed 6-[3-(2,3-dihydro-1-benzofuran-6-sulfonamido)-2,6-difluorophenyl]-7-fluoro-N-methyl-1-[[2-(trimethylsilyl)ethoxy]methyl]indazole-3-carboxamide (160 mg, 0.25 mmol, 1 equiv) in DCM (4 mL), followed by TFA (1 mL). The solution was stirred for 2 hr at room temperature then diluted with 10 mL of water. The pH was adjusted to 8 with NaHCHO₃ (2 mmol/L) and the resulting solution was extracted with 2×10 mL of DCM. The extracts were dried (Na₂SO₄) and concentrated under vacuum. The crude product was purified by Prep-HPLC with the following conditions: Water (0.1% FA) and ACN (30.0% ACN up to 65.0% in 7 min). 6-[3-(2,3-Dihydro-1-benzofuran-6-sulfonamido)-2,6-difluorophenyl]-7-fluoro-N-methyl-1-[[2-(trimethylsilyl)ethoxy]methyl]indazole-3-carboxamide (52 mg 33% yield) was isolated as a white solid. LCMS (ES, m/z): [M+H]⁺: 503. ¹H NMR (300 MHz, DMSO-d₆) δ 14.31 (s, 1H), 10.21 (s, 1H), 8.51 (d, J=4.8 Hz, 1H), 8.05 (d, J=8.4 Hz, 1H), 7.45-7.32 (m, 2H), 7.31-7.16 (m, 2H), 7.10 (dd, J=8.4, 5.8 Hz, 1H), 7.03 (d, J=1.6 Hz, 1H), 4.61 (t, J=8.8 Hz, 2H), 3.26 (t, J=9.2 Hz, 2H), 2.84 (d, J=4.7 Hz, 3H).

### Example 104—Synthesis of 6-[3-(6-cyano-1-hydroxy-2,3-dihydro-1H-indene-4-sulfonamido)-2,6-difluorophenyl]-7-fluoro-N-methyl-1H-indazole-3-carboxamide

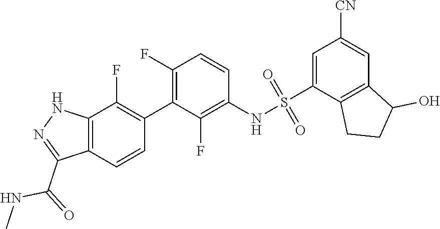 [SMILES: CNC(=O)c1n[nH]c2c(F)c(-c3c(F)ccc(NS(=O)(=O)c4cc(C#N)cc5c4CCC5O)c3F)ccc12]

### Part I—Synthesis of Ethyl 3-(4-bromo-2-fluorophenyl)propanoate

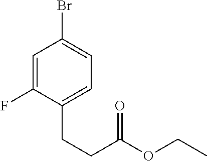

Into a 500-mL 3-necked round-bottom flask purged and maintained with an inert atmosphere of nitrogen, was placed 4-bromo-2-fluoro-1-iodobenzene (20 g, 67 mmol, 1 equiv), DMF (200 mL), 3,3-diethoxy-1-propene (11.3 g, 86 mmol, 1.3 equiv), tetrabutylammonium chloride (18.5 g, 67 mmol, 1 equiv), DIEA (23 g, 179 mmol, 2.7 equiv) and Pd(AcO)₂ (750 mg, 3 mmol, 0.05 equiv). The resulting solution was stirred for 2 h at 90° C. in an oil bath. The reaction mixture was cooled to 25° C. with a water/ice bath and the solution was diluted with 600 mL of H₂O. The resulting mixture was extracted with 2×200 mL of ethyl acetate and the organic layers combined. The organics were washed with 100 mL of brine. The mixture was dried over anhydrous sodium sulfate and concentrated under vacuum. The residue was applied onto a silica gel column, eluting with PE/EA=95/5. Ethyl 3-(4-bromo-2-fluorophenyl)propanoate (14.5 g, 50% yield) was isolated as a light yellow oil. LCMS (ES, m/z): [M+H]⁺: 275.

### Part II—Synthesis of 3-(4-Bromo-2-fluorophenyl) propanoic acid

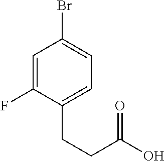 [SMILES: O=C(O)CCc1ccc(Br)cc1F]

Into a 1000-mL round-bottom flask, was placed ethyl 3-(4-bromo-2-fluorophenyl)propanoate (16.5 g, 60 mmol, 1 equiv), THE (120 mL), MeOH (120 mL) and 4N aqueous NaOH (120 mL, 480 mmol). The resulting solution was stirred for 2 h at 50° C. in an oil bath. The reaction mixture was concentrated under vacuum and the residue extracted with 2×100 mL of ethyl acetate. The pH of the aqueous layer was acidified with 4N HCl. The resulting suspension was extracted with 3×100 mL of ethyl acetate and the organic layers combined. The organics were washed with 100 mL of brine, dried over anhydrous sodium sulfate and concentrated under vacuum. The residue was applied onto a silica gel column, eluting with 0-40% EA/PE. 3-(4-Bromo-2-fluorophenyl) propanoic acid (11.5 g, 78% yield) was isolated as a white solid.

### Part III—Synthesis of 6-bromo-4-fluoro-2,3-dihydroinden-1-one

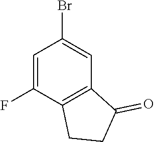 [SMILES: O=C1CCc2c(F)cc(Br)cc21]

To a stirred mixture of 3-(4-bromo-2-fluorophenyl)propanoic acid (11.5 g, 47 mmol, 1 equiv) in DCM (200 mL) was added oxalyl chloride (11.8 g, 93 mmol, 2 equiv) dropwise at room temperature under nitrogen atmosphere. The resulting mixture was stirred for 4 h then concentrated under reduced pressure. The residue was dissolved in DCM (200 mL). To the above mixture was added AlCl₃ (18.6 g, 140 mmol, 3 equiv) in portions at room temperature. The resulting mixture was stirred for additional 3 h at 40° C. Further AlCl₃ (18.6 g, 140 mmol, 3 equiv) was added in portions. The resulting mixture was stirred overnight at 40° C. The reaction mixture was diluted with NH₄Cl (300 mL) and extracted with CH₂Cl₂ (3×200 mL). The combined organic layers were washed with brine (100 mL) and dried over anhydrous Na₂SO₄. After filtration, the filtrate was concentrated under reduced pressure. The residue was purified by silica gel column chromatography with EA/PE=1/2 to afford 6-bromo-4-fluoro-2,3-dihydroinden-1-one (6.5 g, 61% yield) as a white solid.

### Part IV—Synthesis of 7-Fluoro-3-oxo-1,2-dihydroindene-5-carbonitrile

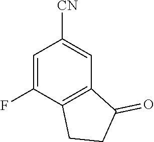 [SMILES: N#Cc1cc(F)c2c(c1)C(=O)CC2]

Into a 100-mL round-bottom flask purged and maintained with an inert atmosphere of nitrogen, was placed 6-bromo-4-fluoro-2,3-dihydroinden-1-one (4.6 g, 20.1 mmol, 1 equiv), NMP (50 mL) and Cu(CN)₂ (4.7 g, 40 mmol, 2 equiv). The resulting solution was stirred overnight at 175° C. The cooled reaction mixture was diluted with 200 mL of H₂O and extracted with 3×50 mL of ethyl acetate. The combined extracts were washed with 1×100 mL of brine, dried over anhydrous sodium sulfate and concentrated under vacuum. The residue was applied onto a silica gel column, eluting with 50-70% THF/PE. 7-Fluoro-3-oxo-1,2-dihydroindene-5-carbonitrile (1.5 g, 43% yield) was isolated as a light yellow solid.

### Part V—Synthesis of 7-(benzylsulfanyl)-3-oxo-1,2-dihydroindene-5-carbonitrile

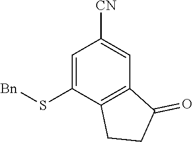 [SMILES: N#Cc1cc(SCc2ccccc2)c2c(c1)C(=O)CC2]

Into a 4 mL vial were added 7-fluoro-3-oxo-1,2-dihydroindene-5-carbonitrile (450 mg, 2.6 mmol, 1 equiv) and ACN (15 mL) at room temperature. To the stirred solution was added Cs₂CO₃ (920 mg, 2.8 mmol, 1.1 equiv) and benzyl mercaptan (478 mg, 3.9 mmol, 1.5 equiv) at room temperature. The resulting mixture was stirred overnight at room temperature. The reaction mixture was concentrated under reduced pressure. The residue was purified by silica gel column chromatography, eluting with CH₂Cl₂/MeOH (9:1) to afford 7-(benzylsulfanyl)-3-oxo-1,2-dihydroindene-5-carbonitrile (550 mg, 77% yield) as a white solid.

### Part VI—Synthesis of 7-(benzylsulfanyl)-3-hydroxy-2,3-dihydro-1H-indene-5-carbonitrile

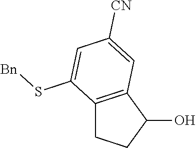 [SMILES: N#Cc1cc(SCc2ccccc2)c2c(c1)C(O)CC2]

Into a 100 mL round-bottom flask were added 7-(benzylsulfanyl)-3-oxo-1,2-dihydroindene-5-carbonitrile (550 mg, 2 mmol, 1 equiv) and MeOH (15 mL) at room temperature. To the solution was added NaBH₄ (97 mg, 2.6 mmol, 1.3 equiv) at room temperature. The resulting mixture was stirred for 1 h at room temperature, then diluted with water (50 mL). The resulting mixture was extracted with EtOAc (3×50 mL). The combined organic layers were washed with brine (30 mL), dried over anhydrous Na₂SO₄. After filtration, the filtrate was concentrated under reduced pressure. The residue was purified by silica gel column chromatography, eluting with CH₂Cl₂/MeOH (9:1) to afford 7-(benzylsulfanyl)-3-hydroxy-2,3-dihydro-1H-indene-5-carbonitrile (560 mg, crude) as a light grey solid.

### Part VII—Synthesis of 4-(benzylsulfanyl)-6-cyano-2,3-dihydro-1H-inden-1-yl acetate

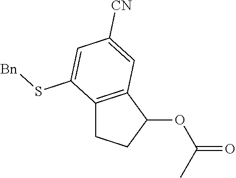 [SMILES: CC(=O)OC1CCc2c(SCc3ccccc3)cc(C#N)cc21]

Into a 2 mL vial were added 7-(benzylsulfanyl)-3-hydroxy-2,3-dihydro-1H-indene-5-carbonitrile (400 mg, 1.4 mmol, 1 equiv) and DCM (10 mL) at room temperature. To the stirred solution was added TEA (288 mg, 2.8 mmol, 2 equiv) and acetyl chloride (167 mg, 2.1 mmol, 1.5 equiv) dropwise at room temperature under nitrogen atmosphere. The reaction mixture was stirred for 1.5 h at room temperature, then quenched by the addition of MeOH (5 mL). The resulting mixture was concentrated under reduced pressure. The residue was purified by silica gel column chromatography, eluting with PE/EtOAc (8:1) to afford 4-(benzylsulfanyl)-6-cyano-2,3-dihydro-1H-inden-1-yl acetate (360 mg, 78%) as a white solid.

### Part VIII—Synthesis of 4-(chlorosulfonyl)-6-cyano-2,3-dihydro-1H-inden-1-yl acetate

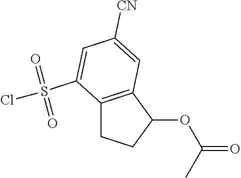 [SMILES: CC(=O)OC1CCc2c1cc(C#N)cc2S(=O)(=O)Cl]

Into a 20 mL vial were added 4-(benzylsulfanyl)-6-cyano-2,3-dihydro-1H-inden-1-yl acetate (400 mg, 1.2 mmol, 1 equiv) and MeCN (4 mL). To the stirred mixture was added 1M HCl (1.2 mL, 33 mmol, 32 equiv) and NCS (661 mg, 4.8 mmol, 4 equiv) in portions. The resulting mixture was diluted with water (10 mL) when the reaction was shown to be complete. The resulting mixture was extracted with EtOAc (3×10 mL). The combined organic layers were washed with brine (5 mL), dried over anhydrous Na₂SO₄. After filtration, the filtrate was concentrated under reduced pressure. The crude product was used in the next step directly without further purification.

### Part IX—Synthesis of methyl 6-[3-[1-(acetyloxy)-6-cyano-2,3-dihydro-1H-indene-4-sulfonamido]-2,6-difluorophenyl]-7-fluoro-1-[[2-(trimethylsilyl)ethoxy]methyl] indazole-3-carboxylate

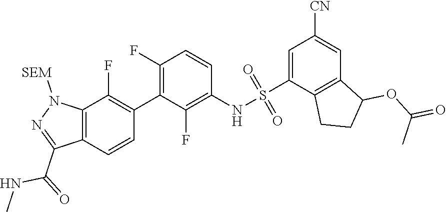 [SMILES: CNC(=O)c1nn(COCC[Si](C)(C)C)c2c(F)c(-c3c(F)ccc(NS(=O)(=O)c4cc(C#N)cc5c4CCC5OC(C)=O)c3F)ccc12]

Into a 20 mL vial were added 4-(chlorosulfonyl)-6-cyano-2,3-dihydro-1H-inden-1-yl acetate (366 mg, 1.2 mmol, 1 equiv) and DCM (5 mL). To the stirred mixture was added pyridine (483 mg, 6 mmol, 5 equiv) and methyl 6-(3-amino-2,6-difluorophenyl)-7-fluoro-1-[[2-(trimethylsilyl)ethoxy]methyl]indazole-3-carboxylate (333 mg, 0.7 mmol, 0.6 equiv) dropwise at room temperature. The resulting mixture was concentrated under reduced pressure. The residue was purified by silica gel column chromatography, eluted with PE/THF (1:1). The crude product was purified by Prep-HPLC with the following conditions: Column: welch Vltimate XB-C18, 50×250 mm, 10 μm, mobile phase: 90 mL/min, Mobile Phase A: 0.1% FA in Water, Mobile Phase B: ACN (30% up to 70% in 15 min) to afford methyl 6-[3-[1-(acetyloxy)-6-cyano-2,3-dihydro-1H-indene-4-sulfonamido]-2,6-difluorophenyl]-7-fluoro-1-[[2-(trimethylsilyl)ethoxy]methyl] indazole-3-carboxylate (55 mg, 6%) as a white solid. LCMS (ES, m/z): [M+H]⁺: 714.

### Part X—Synthesis of 6-[3-(6-cyano-1-hydroxy-2,3-dihydro-1H-indene-4-sulfonamido)-2,6-difluorophenyl]-7-fluoro-N-methyl-1H-indazole-3-carboxamide

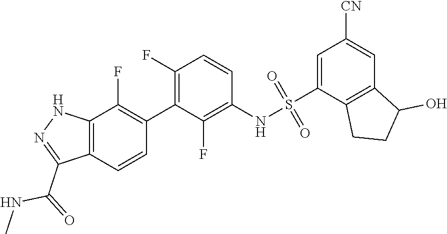 [SMILES: CNC(=O)c1n[nH]c2c(F)c(-c3c(F)ccc(NS(=O)(=O)c4cc(C#N)cc5c4CCC5O)c3F)ccc12]

A solution of 6-cyano-4-([2,4-difluoro-3-[7-fluoro-3-(methylcarbamoyl)-1-[[2-(trimethylsilyl)ethoxy] methyl] indazol-6-yl]phenyl]sulfamoyl)-2,3-dihydro-1H-inden-1-yl acetate (55 mg, 0.08 mmol, 1 equiv) and TBAF (0.6 mL, 0.6 mmol, 7.1 equiv, 1M in THF) was stirred for 4 h at 65° C. The reaction was quenched with sat. NH₄Cl (aq.) and was extracted with EtOAc (3×10 mL). The combined organic layers were washed with water (2×10 mL), dried over anhydrous Na₂SO₄. After filtration, the filtrate was concentrated under reduced pressure. The crude product was purified by Prep-HPLC with the following conditions Column, welch Vltimate XB-C18, 50×250 mm, 10 μm mobile phase, Mobile Phase A: 0.1% FA in Water, Mobile Phase B: ACN (10% up to 50% in 15 min) to afford 6-[3-(6-cyano-1-hydroxy-2,3-dihydro-1H-indene-4-sulfonamido)-2,6-difluorophenyl]-7-fluoro-N-methyl-1H-indazole-3-carboxamide (22.4 mg, 54%) as a white solid. LCMS (ES, m/z): [M+H]⁺: 542. ¹H NMR (300 MHz, DMSO-d₆) δ 14.31 (s, 1H), 10.52 (s, 1H), 8.51 (d, J=4.8 Hz, 1H), 8.09-7.97 (m, 2H), 7.93 (d, J=7.8 Hz, 1H), 7.40 (td, J=8.8, 5.9 Hz, 1H), 7.30 (t, J=9.0 Hz, 1H), 7.07 (s, 1H), 5.09 (t, J=6.8 Hz, 1H), 3.20 (s, 1H), 3.00-2.70 (m, 4H), 2.35 (s, 1H), 1.77 (s, 1H).

### Example 105—Synthesis of 6-[2,6-difluoro-3-(6-fluoro-1-hydroxy-2,3-dihydro-1H-indene-4-sulfonamido)phenyl]-7-fluoro-N-methyl-1H-pyrazolo[4,3-c]pyridine-3-carboxamide

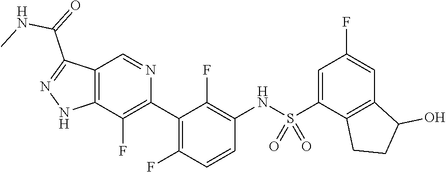 [SMILES: CNC(=O)c1n[nH]c2c(F)c(-c3c(F)ccc(NS(=O)(=O)c4cc(F)cc5c4CCC5O)c3F)ncc12]

### Part I—Synthesis of 4-(benzylsulfanyl)-6-fluoro-2,3-dihydro-1H-inden-1-ol

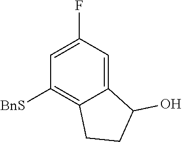

To a stirred solution of 4-(benzylsulfanyl)-6-fluoro-2,3-dihydroinden-1-one (600 mg, 2.2 mmol, 1 equiv) in MeOH (12 mL) was added NaBH₄ (416 mg, 11 mmol, 5 equiv) at 0 degrees C. The reaction was then stirred at room temperature for 30 min and concentrated. The residue was diluted with DCM (5 mL), and this was washed with H₂O (3×5 mL). The organic layer was dried (MgSO₄), concentrated and the residue was purified by silica gel column chromatography, eluting with PE:EA (3:1) to afford 4-(benzylsulfanyl)-6-fluoro-2,3-dihydro-1H-inden-1-ol (0.6 g, 99% yield) as a yellow solid. LCMS (ES, m/z): [M+H]⁺: 275.

### Part II—Synthesis of 4-(Benzylsulfanyl)-6-fluoro-2,3-dihydro-1H-inden-1-yl acetate

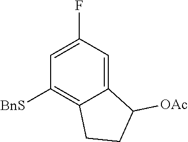

Into a 50-mL 3-necked round-bottom flask, was placed 4-(benzylsulfanyl)-6-fluoro-2,3-dihydro-1H-inden-1-ol (680 mg, 2.5 mmol, 1 equiv), DCM (20 mL), TEA (301 mg, 3 mmol, 1.2 equiv) and DMAP (30 mg, 0.25 mmol, 0.1 equiv). This was followed by the addition of acetic anhydride (380 mg, 3.7 mmol, 1.5 equiv) dropwise with stirring at 0 degrees C. The resulting solution was stirred for 2 h and quenched by the addition of 50 mL of water. The resulting solution was extracted with 3×30 mL of dichloromethane. The combined organics were washed with 50 ml of brine, dried over anhydrous sodium sulfate and concentrated under vacuum. The residue was applied onto a silica gel column, eluting with ethyl acetate/petroleum ether (1:3). 4-(Benzylsulfanyl)-6-fluoro-2,3-dihydro-1H-inden-1-yl acetate (580 mg, 74% yield) was isolated as a white solid. LCMS (ES, m/z): [M+H]⁺: 317.

### Part III—Synthesis of 4-(Chlorosulfonyl)-6-fluoro-2,3-dihydro-1H-inden-1-yl acetate

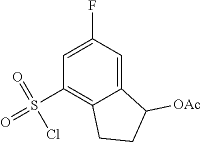 [SMILES: CC(=O)OC1CCc2c1cc(F)cc2S(=O)(=O)Cl]

Into a 50-mL 3-necked round-bottom flask, was placed 4-(benzylsulfanyl)-6-fluoro-2,3-dihydro-1H-inden-1-yl acetate (520 mg, 1.6 mmol, 1 equiv), CH₃CN (10 mL) and 6M HCl (5 mL). This was followed by the addition of NCS (878 mg, 6.6 mmol, 4 equiv) in portions at 0 degrees C. The resulting solution was stirred for 30 min at 0 degrees C., and was then quenched by the addition of 50 mL of water. The resulting solution was extracted with 3×30 mL of ethyl acetate. The organic layer was washed with 50 ml of water and 50 mL of brine. The mixture was dried over anhydrous sodium sulfate and concentrated under vacuum. 4-(Chlorosulfonyl)-6-fluoro-2,3-dihydro-1H-inden-1-yl acetate (450 mg crude) of was isolated as a light yellow oil and used in the next step directly without further purification. LCMS (ES, m/z): [M+H]⁺: 293.

### Part IV—Synthesis of 4-([2,4-difluoro-3-[7-fluoro-3-(methylcarbamoyl)-1H-pyrazolo[4,3-c]pyridin-6-yl]phenyl] sulfamoyl)-6-fluoro-2,3-dihydro-1H-inden-1-yl acetate

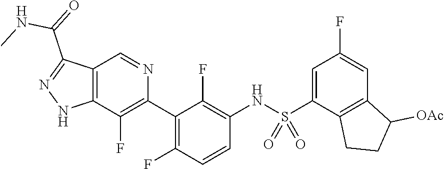 [SMILES: CNC(=O)c1n[nH]c2c(F)c(-c3c(F)ccc(NS(=O)(=O)c4cc(F)cc5c4CCC5OC(C)=O)c3F)ncc12]

To a stirred solution of 6-(3-amino-2,6-difluorophenyl)-7-fluoro-N-methyl-1H-pyrazolo[4,3-c]pyridine-3-carboxamide (See Example 66) (105 mg, 0.3 mmol, 1 equiv) in pyridine (5 mL) was added 4-(chlorosulfonyl)-6-fluoro-2,3-dihydro-1H-inden-1-yl acetate (115 mg, 0.4 mmol, 1.2 equiv) in portions at room temperature. The resulting mixture was stirred for 0.5 h then concentrated under reduced pressure. The residue was diluted with EA (50 mL). The combined organic layers were washed with brine (2×5 mL), dried over anhydrous sodium sulfate. After filtration, the filtrate was concentrated under reduced pressure to give 4-([2,4-difluoro-3-[7-fluoro-3-(methylcarbamoyl)-1H-pyrazolo[4,3-c]pyridin-6-yl]phenyl] sulfamoyl)-6-fluoro-2,3-dihydro-1H-inden-1-yl acetate (100 mg) as a yellow oil which was used in the next step directly without further purification.

### Part V—Synthesis of 6-[2,6-difluoro-3-(6-fluoro-1-hydroxy-2,3-dihydro-1H-indene-4-sulfonamido)phenyl]-7-fluoro-N-methyl-1H-pyrazolo[4,3-c]pyridine-3-carboxamide

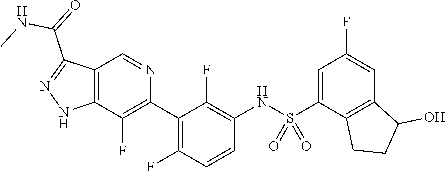 [SMILES: CNC(=O)c1n[nH]c2c(F)c(-c3c(F)ccc(NS(=O)(=O)c4cc(F)cc5c4CCC5O)c3F)ncc12]

To a stirred solution of 4-([2,4-difluoro-3-[7-fluoro-3-(methylcarbamoyl)-1H-pyrazolo[4,3-c]pyridin-6-yl]phenyl] sulfamoyl)-6-fluoro-2,3-dihydro-1H-inden-1-yl acetate (100 mg, 0.2 mmol, 1 equiv) in THE (2 mL) was added a solution of NaOH (14 mg, 0.35 mmol, 2 equiv) in H₂O (2 mL) dropwise at 0 degrees C. The resulting mixture was stirred for 0.5 h at room temperature, then concentrated under reduced pressure. The residue was purified by prep-HPLC with the following conditions (IntelFlash-1): welch Vltimate XB-C18, 50×250 mm, 10 μm; mobile phase, 0.1% FA and MeCN, from 15% increasing to 50% in 10 min; Detector, 220 nm. It afforded 6-[2,6-difluoro-3-(6-fluoro-1-hydroxy-2,3-dihydro-1H-indene-4-sulfonamido)phenyl]-7-fluoro-N-methyl-1H-pyrazolo[4,3-c]pyridine-3-carboxamide (50 mg, 53.9%) as a white solid. LCMS (ES, m/z): [M+H]⁺: 536. ¹H NMR (300 MHz, DMSO-d₆) δ 9.29 (d, J=2.3 Hz, 1H), 8.71 (d, J=4.9 Hz, 1H), 7.48-7.30 (m, 3H), 7.25 (t, J=8.9 Hz, 1H), 5.51 (d, J=6.0 Hz, 1H), 5.03 (q, J=6.6 Hz, 1H), 3.10 (s, 1H), 2.86 (d, J=4.7 Hz, 3H), 2.73 (dd, J=16.5, 8.6 Hz, 1H), 2.40-2.29 (m, 1H), 1.85-1.67 (m, 1H).

### Example 106—Biochemical Assay for Inhibition of GCN2

Exemplary compounds from the above Examples were tested for ability to inhibit GCN2 activity using a time resolved fluorescence energy transfer (TR-FRET) assay. Assay procedures and results are described below for Example 1-4BB.

### Part I—Procedures for TR-FRET Assay

GCN2 protein was obtained from Carna Biosciences (cat #05-153). The protein was diluted in assay buffer (50 mM HEPES pH 7.5, 10 mM MgCl₂, 1 mM ethylene glycol-bis(β-aminoethyl ether)-N,N,N′,N′-tetraacetic acid (EGTA), 2 mM dithiothreitol (DTT), 0.01% Brij-35) to obtain a 4×GCN2 concentration of 16 nM (2 nM final concentration) and 2.5 μL was plated in a 384-well white assay plate. Compounds were diluted to 4× final test concentration in assay buffer containing 4% DMSO, and a 2.5 μL aliquot was added to appropriate wells (final DMSO concentration of 1%). GFP-eIF2α protein was obtained from ThermoFisher (cat #PV4809). The protein was diluted in assay buffer to a 2× concentration of 200 nM along with 2 mM ATP (final concentration of 100 nM GFP-eIF2α and 1 mM ATP) and a 5 μL aliquot was added to each well containing the GCN2 protein and test compound. The plate was incubated in the dark at 25° C. for 1.5 hours, shaking at 1250 rpm. Tb-anti P-eIF2α (ThermoFisher cat #PV4810) was diluted to a 2× concentration of 4 nM in TR-FRET Dilution Buffer (ThermoFisher cat #PV3574) and 20 mM EDTA. 10 μL of the Tb-anti P-eIF2α solution was added to the TR-FRET reaction (final concentration of 2 nM Tb-anti P-eIF2α, 10 mM EDTA). The plate was incubated in the dark for 2 h at 25° C. shaking at 600 rpm. The FRET signal from the plate was read on a Tecan SPARK plate reader:

Label 1: Excitation: 340 nm, bandwidth 30 nm; Emission: 495 nm, bandwidth 10 nm. Lag time: 100 μsec. Integration time: 400 μsec. Flashes: 30.

Label 2: Excitation: 340 nm, bandwidth 30 nm; Emission: 520 nm, bandwidth 25 nm. Lag time: 100 μsec. Integration time: 400 μsec. Flashes: 30

The data were analyzed using GraphPad Prism employing a 4-parameter sigmoidal curve fit.

### Example 107

Exemplary compounds from the above Examples were tested for ability to inhibit GCN2 activity using a time resolved fluorescence energy transfer (TR-FRET) assay. Assay procedures and results are described below for Examples 40-72.

### PartI—Procedure for TR-FRET Assay

Test compounds were serially diluted to 11 concentrations by 3-fold dilution in DMSO and 10 nL of stock was plated into 384 well white assay plate. DMSO was used as a vehicle control. GCN2 Kinase (Carna Biosciences, #05-153) was diluted in assay buffer (ThermoFisher Scientific, #PV6135) and 2 mM dithiothreitol (DTT) to obtain a final GCN2 concentration of 2 nM and 5 μL was added into wells. GFP-eIF2 alpha Substrate (Invitrogen, #PV4809) was diluted in 1× assay buffer to a 2× concentration of 200 nM and 300 μM ATP (final concentration of 100 nM GFP-eIF2 alpha Substrate and 150 μM ATP), in the presence of 2 mM DTT and 5 μl this solution was added in to each well containing GCN2 kinase and compounds. The plate was incubated at 25° C. for 1.5 hours shaking at 1250 rpm in the dark. Tb-anti-p-eIF2αantibody (Invitrogen, PV4815) was diluted in TR-FRET Dilution Buffer (ThermoFisher Scientific, #PV3574) to a concentration of 1 nM and 10 μL was added to the TR-FRET reaction. The plate was incubated at 25° C. in the dark shaking at 600 rpm for 2 hours. The signal from the plate was read on Envision (PerkinElmer) plate reader.

### Part II—Results

Experimental results are provided in Table 10 below. The symbol “++++” indicates an IC₅₀ less than 0.5 μM. The symbol “+++” indicates an IC₅₀ in the range of 0.5 μM to 2.5 μM. The symbol “++” indicates an IC₅₀ in the range of greater than 2.5 μM to 10 μM. The symbol “+” indicates an IC₅₀ greater than 10 μM. The symbol “N/A” indicates that no data was available.

### Example 108—Biochemical Assay for Inhibition of GCN2

Exemplary compounds from the above Examples were tested for ability to inhibit GCN2 activity using a time resolved fluorescence energy transfer (TR-FRET) assay. Assay procedures and results are described below for Examples 73-105.

### PartI—Procedures for TR-FRET Assay

GCN2 protein was obtained from Carna Biosciences (cat #05-153). The protein was diluted in assay buffer (50 mM HEPES pH 7.5, 10 mM MgCl₂, 1 mM ethylene glycol-bis(β-aminoethyl ether)-N,N,N′,N′-tetraacetic acid (EGTA), 2 mM dithiothreitol (DTT), 0.01% Brij-35) to obtain a 4×GCN2 concentration of 16 nM (2 nM final concentration) and 2.5 μL was plated in a 384-well white assay plate. Compounds were diluted to 4× final test concentration in assay buffer containing 4% DMSO, and a 2.5 μL aliquot was added to appropriate wells (final DMSO concentration of 1%). GFP-eIF2α protein was obtained from ThermoFisher (cat #PV4809). The protein was diluted in assay buffer to a 2× concentration of 200 nM along with 2 mM ATP (final concentration of 100 nM GFP-eIF2α and 1 mM ATP) and a 5 μL aliquot was added to each well containing the GCN2 protein and test compound. The plate was incubated in the dark at 25° C. for 1.5 hours, shaking at 1250 rpm. Tb-anti P-eIF2α (ThermoFisher cat #PV4810) was diluted to a 2× concentration of 4 nM in TR-FRET Dilution Buffer (ThermoFisher cat #PV3574) and 20 mM EDTA. 10 μL of the Tb-anti P-eIF2α solution was added to the TR-FRET reaction (final concentration of 2 nM Tb-anti P-eIF2α, 10 mM EDTA). The plate was incubated in the dark for 2 h at 25° C. shaking at 600 rpm. The FRET signal from the plate was read on a Tecan SPARK plate reader:

Label 1: Excitation: 340 nm, bandwidth 30 nm; Emission: 495 nm, bandwidth 10 nm. Lag time: 100 μsec. Integration time: 400 μsec. Flashes: 30.

Label 2: Excitation: 340 nm, bandwidth 30 nm; Emission: 520 nm, bandwidth 25 nm. Lag time: 100 μsec. Integration time: 400 μsec. Flashes: 30

The data were analyzed using GraphPad Prism employing a 4-parameter sigmoidal curve fit.

### Part II—Results

Experimental results are provided in Table 11 below. The symbol “++++” indicates an IC₅₀ less than 0.5 μM. The symbol “+++” indicates an IC₅₀ in the range of 0.5 μM to 2.5 μM. The symbol “++” indicates an IC₅₀ in the range of greater than 2.5 μM to 10 μM. The symbol “+” indicates an IC₅₀ greater than 10 μM. The symbol “N/A” indicates that no data was available.

TABLE 8

| Title Compound | |
|---|---|
| from Example No. | IC₅₀ |
| 1 | +++ |
| 2 | ++++ |
| 3 | ++++ |
| 4 | ++++ |
| 5 | ++++ |
| 6 | ++++ |
| 7 | +++ |
| 8 | +++ |
| 2A | ++++ |
| 2B | +++ |
| 2C | ++ |
| 2D | ++ |
| 2E | +++ |
| 2F | ++++ |
| 2G | + |
| 2H | +++ |
| 2I | +++ |
| 2J | ++++ |
| 2K | +++ |
| 2L | ++ |
| 2M | ++++ |
| 2N | ++ |
| 2O | ++++ |
| 2P | ++++ |
| 2Q | +++ |
| 2R | +++ |
| 2S | ++ |
| 2T | ++++ |
| 2U | ++++ |
| 2V | ++++ |
| 2W | ++ |
| 2X | ++ |
| 2Y | ++ |
| 2Z | ++++ |
| 2AA | ++++ |
| 2AB | ++++ |
| 2AC | ++ |
| 2AD | ++++ |
| 2AE | + |
| 2AF | ++++ |
| 2AG | ++++ |
| 2AH | ++++ |
| 2AI | ++++ |
| 2AJ | ++ |
| 2AK | ++++ |
| 2AL | ++ |
| 2AM | +++ |
| 2AN | ++++ |
| 2AO | ++ |
| 2AP | ++++ |
| 2AQ | ++++ |
| 2AR | +++ |
| 2AS | +++ |
| 2AT | ++++ |
| 2AU | +++ |
| 2AV | ++++ |
| 2AW | ++++ |
| 2AX | ++++ |
| 2AY | ++++ |
| 2AZ | ++++ |
| 2BA | ++++ |
| 2BB | ++++ |
| 2BC | ++++ |
| 2BD | ++++ |
| 2BE | ++++ |
| 2BF | +++ |
| 2BG | ++++ |
| 2BH | ++++ |
| 2BI | ++++ |
| 2BJ | ++++ |
| 2BK | ++++ |
| 2BL | ++++ |
| 2BM | ++++ |
| 2BN | ++++ |
| 2BO | +++ |
| 2BP | ++++ |
| 2BQ | ++++ |
| 2BR | ++++ |
| 2BS | ++++ |
| 2BT | ++++ |
| 2BU | ++++ |
| 2BV | +++ |
| 2BW | ++++ |
| 2BX | +++ |
| 2BY | +++ |
| 2BZ | ++++ |
| 2CA | +++ |
| 2CB | ++++ |
| 2CC | ++ |
| 2CD | +++ |
| 2CE | ++++ |
| 2CF | ++++ |
| 2CG | ++++ |
| 2CH | ++++ |
| 2CI | ++++ |
| 2CJ | ++++ |
| 2CK | ++++ |
| 2CL | ++++ |
| 2CM | ++++ |
| 2CN | +++ |
| 2CO | ++ |

TABLE 9

| Title Compound | |
|---|---|
| from Example No. | IC₅₀ |
| 10 | ++ |
| 11 | + |
| 12 | + |
| 13 | ++++ |
| 14 | +++ |
| 15 | ++++ |
| 17 | ++++ |
| 18 | +++ |
| 19 | ++++ |
| 20 | ++++ |
| 21 | ++++ |
| 22 | +++ |
| 23 | +++ |
| 24 | + |
| 25 | ++ |
| 26 | ++++ |
| 27 | ++++ |
| 28 | ++++ |
| 29 | ++ |
| 30 | ++++ |
| 31 | +++ |
| 32 | ++++ |
| 35 | ++++ |
| 36 | ++++ |
| 38 | ++++ |
| 3A | ++++ |
| 3B | ++ |
| 3C | ++++ |
| 3D | ++++ |
| 3E | ++++ |
| 3F | ++++ |
| 3G | + |
| 3H | ++++ |
| 3I | ++++ |
| 3J | ++ |
| 3K | ++++ |
| 3L | + |
| 3M | + |
| 3N | +++ |
| 3O | ++++ |
| 3P | + |
| 3Q | +++ |
| 3R | ++++ |
| 3S | ++++ |
| 3T | ++++ |
| 3U | ++++ |
| 3V | ++++ |
| 3W | ++++ |
| 3X | ++++ |
| 3Y | +++ |
| 3Z | ++++ |
| 3AA | ++ |
| 3AB | ++++ |
| 3AC | +++ |
| 3AD | ++++ |
| 3AF | ++++ |
| 3AG | ++++ |
| 3AH | ++++ |
| 3AI | ++++ |
| 3AJ | ++++ |
| 3AK | ++++ |
| 3AL | ++++ |
| 3AM | +++ |
| 3AN | ++++ |
| 3AO | +++ |
| 3AP | ++++ |
| 3AQ | ++++ |
| 3AR | ++ |
| 3AS | ++++ |
| 3AT | ++++ |
| 3AU | +++ |
| 3AV | ++++ |
| 3AW | ++++ |
| 3AX | +++ |
| 3AY | +++ |
| 3AZ | ++++ |
| 3BA | ++++ |
| 3BB | ++++ |
| 3BC | ++++ |
| 3BD | ++++ |
| 3BE | ++++ |
| 3BF | ++++ |
| 3BG | ++++ |
| 3BH | +++ |
| 3BI | +++ |
| 3BJ | +++ |
| 3BK | ++++ |
| 3BL | ++++ |
| 3BM | ++++ |
| 3BN | ++++ |
| 3BO | ++++ |
| 3BP | ++++ |
| 3BQ | +++ |
| 3BR | +++ |
| 3BS | ++++ |
| 3BT | +++ |
| 3BU | +++ |
| 3BV | ++++ |
| 3BW | ++ |
| 3BX | ++++ |
| 3BY | ++++ |
| 3BZ | ++++ |
| 3CA | ++++ |
| 3CB | + |
| 3CC | + |
| 3CD | ++++ |
| 3CE | + |
| 3CF | ++++ |
| 3CG | ++++ |
| 3CH | ++++ |
| 3CI | +++ |
| 3CJ | +++ |
| 3CK | ++++ |
| 3CL | + |
| 3CM | + |
| 3CN | ++ |
| 3CO | +++ |
| 3CP | +++ |
| 3CQ | ++++ |
| 3CR | +++ |
| 3CS | ++++ |
| 3CT | ++++ |
| 3CU | ++++ |
| 3CV | ++++ |
| 3CW | ++++ |
| 3CX | +++ |
| 3CY | +++ |
| 3CZ | +++ |
| 3DA | ++ |
| 3DB | + |
| 3DC | +++ |
| 3DD | ++++ |
| 3DE | ++ |
| 3DF | +++ |
| 3DG | ++++ |
| 3DH | ++++ |
| 3DI | ++++ |
| 3DJ | ++++ |
| 3DK | ++++ |
| 3DL | +++ |
| 3DM | ++++ |
| 3DN | ++++ |
| 3DO | ++++ |
| 3DP | ++++ |
| 3DQ | ++++ |
| 3DR | ++++ |
| 3DS | ++++ |
| 4A | ++++ |
| 4B | +++ |
| 4C | +++ |
| 4D | ++++ |
| 4E | ++++ |
| 4F | ++++ |
| 4G | ++++ |
| 4H | ++++ |
| 4I | ++++ |
| 4J | ++++ |
| 4K | ++++ |
| 4L | ++++ |
| 4M | ++++ |
| 4N | ++++ |
| 4O | ++++ |
| 4P | ++++ |
| 4Q | +++ |
| 4R | ++++ |
| 4S | ++++ |
| 4T | ++++ |
| 4U | ++++ |
| 4V | ++++ |
| 4W | ++++ |
| 4X | +++ |
| 4Y | ++++ |
| 4Z | ++++ |
| 4AA | ++ |
| 4AB | ++++ |
| 4AC | ++++ |
| 4AD | ++++ |
| 4AE | +++ |
| 4AF | ++++ |
| 4AG | +++ |
| 4AH | ++++ |
| 4AI | ++++ |
| 4AJ | ++++ |
| 4AK | ++++ |
| 4AL | ++++ |
| 4AM | ++++ |
| 4AN | ++++ |
| 4AO | + |
| 4AP | ++++ |
| 4AQ | ++++ |
| 4AR | ++++ |
| 4BB | +++ |

TABLE 10

| Title Compound | |
|---|---|
| from Example No. | IC50 |
| 40 | ++ |
| 41 | +++ |
| 42 | ++++ |
| 43 | N/A |
| 44 | ++++ |
| 45 | ++++ |
| 46 | ++++ |
| 47 | ++++ |
| 48 | ++++ |
| 49 | ++++ |
| 50 | ++++ |
| 51 | ++++ |
| 52 | ++++ |
| 53 | ++++ |
| 54 | ++++ |
| 55 | ++++ |
| 56 | ++++ |
| 57 | ++++ |
| 58 | ++++ |
| 59 | ++++ |
| 60 | ++++ |
| 61 | ++++ |
| 62 | +++ |
| 63 | ++++ |
| 64 | ++++ |
| 65 | ++++ |
| 66 | ++++ |
| 67 | ++++ |
| 68 | +++ |
| 69 | +++ |
| 70 | ++++ |
| 71 | ++++ |
| 72 | ++++ |

TABLE 11

| Title Compound | |
|---|---|
| from Example No. | IC₅₀ |
| 73 | ++++ |
| 74 | ++ |
| 75 | ++ |
| 76 | ++++ |
| 77 | ++ |
| 78 | ++ |
| 79 | ++++ |
| 80 | ++++ |
| 81 | ++++ |
| 82 | ++++ |
| 83 | ++++ |
| 84 | ++++ |
| 85 | ++++ |
| 86 | ++++ |
| 87 | ++++ |
| 88 | ++++ |
| 89 | ++++ |
| 90 | ++++ |
| 91 | ++++ |
| 92 | ++++ |
| 93 | ++++ |
| 94 | +++ |
| 95 | ++++ |
| 96 | ++++ |
| 97 | ++++ |
| 98 | ++++ |
| 99 | ++++ |
| 100 | ++++ |

1. A compound represented by Formula I: 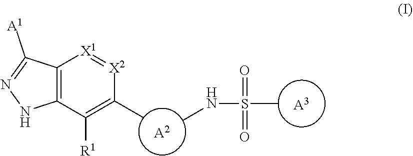
or a pharmaceutically acceptable salt thereof; wherein:
X¹ and X² are independently C(R²) or N, wherein X¹ is N and X² is C(R²), X¹ is C(R²) and X² is N, or both X¹ and X² are C(R²);
R¹ is halogen, hydrogen, C₁₋₄ alkyl, C₁₋₄ fluoroalkyl, or cyano;
R² represents independently for each occurrence hydrogen, halogen, C₁₋₄ alkyl, C₁₋₄ fluoroalkyl, cyano, C₁₋₄ alkoxyl, or hydroxyl;
R³ and R⁴ each represent independently for each occurrence hydrogen, C₁₋₄ alkyl, or C₃₋₇ cycloalkyl; or an occurrence of R³ and R⁴ attached to the same nitrogen atom are taken together with the nitrogen atom to which they are attached to form a 3-7 membered carbocyclyl or heterocyclyl;
R⁵ represents independently for each occurrence hydrogen, C₁₋₄ alkyl, or hydroxyl;
R⁶ represents independently for each occurrence hydrogen, C₁₋₆ alkyl, C₁₋₆ hydroxyalkyl, C₃₋₆ cycloalkyl, 4-7 membered heterocyclyl, 6-10 membered aryl, —(C₁₋₆ alkylene)-N(R³)(R⁴), —(C₁₋₆ alkylene)-N(R³)—C(O)(R⁴), —(C₁₋₆ alkylene)-(5-10 membered heteroaryl), —(C₁₋₆ alkylene)-(C₃₋₆ cycloalkyl), —(C₁₋₆ alkylene)-(5-10 membered heterocycloalkyl), —(C₁₋₆ alkylene)-CO₂R³, —(C₁₋₆ alkylene)-C(O)N(R³)(R⁴), —(C₁₋₆ alkylene)-S(O)₂—(C₁₋₆ alkyl), —(C₁₋₆ alkylene)-O—(C₁₋₆ alkyl), or —(C₁₋₆ alkylene)-CN, wherein the C₁₋₆ alkyl, C₃₋₆ cycloalkyl, —(C₁₋₆ alkylene)-(C₃₋₆ cycloalkyl), 4-7 membered heterocyclyl, and —(C₁₋₆ alkylene)-(5-10 membered heterocycloalkyl) may be optionally substituted with 1, 2, or 3 substituents independently selected from the group consisting cyano, halogen, hydroxyl, oxo, and NH₂, and wherein if the 4-7 membered heterocyclyl and —(C₁₋₆ alkylene)-(5-10 membered heterocycloalkyl) contain a suitable ring nitrogen atom, that ring nitrogen may be optionally substituted by C₁₋₃ alkyl or —C(O)—C₁₋₃ alkyl;
R⁷ is C₁₋₄ alkyl, C₃₋₇ cycloalkyl, or —(C₁₋₆ alkylene)-(C₃₋₇ cycloalkyl);
A¹ is one of the following:
5-10 membered heterocyclyl or 6-10 membered aryl, each of which is optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, —N(R³)(R⁴), —(C₁₋₆ alkylene)-N(R³)(R⁴), —C(O)N(R⁵)(R⁶), and —(C₁₋₆ alkylene)-C(O)N(R⁵)(R⁶); or
—C(O)N(R⁵)(R⁶) or —N(R⁵)C(O)(R⁷);
A² is phenylene or a 5-6 membered heteroarylene, each of which is optionally substituted with 1, 2, or 3 substituents independently selected from the group consisting of halogen, C₁₋₄ alkyl, C₁₋₄ haloalkyl, cyano, C₁₋₄ alkoxyl, C₃₋₅ cycloalkyl, and C₃₋₅ halocycloalkyl; and
A³ is phenyl, —CH₂—(C₃₋₆ cycloalkyl), 7-10 membered bicyclic carbocyclyl, or 5-10 membered heterocyclyl, each of which is optionally substituted with 1, 2, or 3 substituents independently selected from the group consisting of halogen, C₁₋₆ alkyl, C₁₋₆ hydroxyalkyl, C₁₋₄ haloalkyl, C₁₋₄ hydroxyfluoroalkyl, C₃₋₅ cycloalkyl, cyano, hydroxyl, C₁₋₄ alkoxyl, C₁₋₄ fluoroalkoxyl, —N(R³)(R⁴), —N(R³)—C(O)(R⁴), —(C₁₋₆ alkylene)-N(R³)(R⁴), —CO₂H, —CO₂(C₁₋₆ alkyl), —S—(C₁₋₆ alkyl), and —S—(C₁₋₆ fluoroalkyl), wherein each of the 7-10 membered bicyclic carbocyclyl and 5-10 membered heterocyclyl is optionally further substituted by oxo or oxime, and wherein if the 5-10 membered heterocyclyl contains a suitable ring nitrogen atom, that ring nitrogen may be optionally substituted by C₁₋₃ alkyl.

2.-4. (canceled)

5. The compound of claim 1, wherein R¹ is halogen, C₁₋₄ alkyl, C₁₋₄ fluoroalkyl, or cyano.

6-7. (canceled)

8. The compound of claim 1, wherein R² is hydrogen.

9. The compound of claim 1, wherein R³ and R⁴ each represent independently for each occurrence hydrogen or C₁₋₄ alkyl.

10. The compound of claim 1, wherein A¹ is a 5-10 membered unsaturated heterocyclyl optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, —N(R³)(R⁴), and —(C₁₋₆ alkylene)-N(R³)(R⁴).

11. The compound of claim 1, wherein A¹ is a 5-membered heteroaryl optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, —N(R³)(R⁴), and —(C₁₋₆ alkylene)-N(R³)(R⁴).

12.-18. (canceled)

19. The compound of claim 1, wherein A is —C(O)N(R⁵)(R⁶).

20. The compound of claim 19, wherein R⁵ is hydrogen.

21. The compound of claim 19, wherein R⁶ represents individually for each occurrence, hydrogen, C₁₋₆ alkyl or C₁₋₆ hydroxyalkyl.

22. (canceled)

23. The compound of any ne of claim 1, wherein A¹ is —N(R⁵)C(O)(R⁷).

24. The compound of claim 1, wherein R⁷ is C₁₋₄ alkyl.

25. The compound of claim 1, wherein A² is phenylene or a 5-6 membered heteroarylene, each of which is optionally substituted with 1, 2, or 3 substituents independently selected from the group consisting of halogen, C₁₋₄ alkyl, C₁₋₄ haloalkyl, cyano, and C₁₋₄ alkoxyl.

26.-33. (canceled)

34. The compound of claim 1, wherein A³ is a 5-10 membered unsaturated heterocyclyl optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, —N(R³)(R⁴), and —(C₁₋₆ alkylene)-N(R³)(R⁴).

35.-86. (canceled)

87. A compound selected from the group consisting of:
6-(3-benzenesulfonamido-2,6-difluorophenyl)-7-fluoro-N-methyl-1H-indazole-3-carboxamide;
6-[2,6-Difluoro-3-[3-(hydroxymethyl)benzenesulfonamido]phenyl]-7-fluoro-N-methyl-1H-indazole-3-carboxamide;
6-[2,6-difluoro-3-[3-fluoro-5-(hydroxymethyl)benzenesulfonamido]phenyl]-7-fluoro-N-methyl-1H-indazole-3-carboxamide;
6-(3-amino-2-fluorophenyl)-7-fluoro-N-methyl-1-[[2-(trimethylsilyl)ethoxy]methyl]indazole-3-carboxamide;
N-(6-(3-(5-chloro-2-methoxypyridine-3-sulfonamido)-2,6-difluorophenyl)-7-fluoro-1H-indazol-3-yl)acetamide;
6-[3-[5-chloro-2-(difluoromethoxy)pyridine-3-sulfonamido]-2,6-difluorophenyl]-7-fluoro-N-methyl-1H-indazole-3-carboxamide;
6-[3-[5-Cyano-2-(difluoromethoxy)pyridine-3-sulfonamido]-2,6-difluorophenyl]-7-fluoro-N-methyl-1H-indazole-3-carboxamide;
6-[3-(5-chloro-2-methoxypyridine-3-sulfonamido)-2,6-difluorophenyl]-7-fluoro-N,4-dimethyl-1H-indazole-3-carboxamide;
6-[3-(5-Chloro-2-methoxypyridine-3-sulfonamido)-2,6-difluorophenyl]-7-fluoro-N-(2-methylpropyl)-1H-indazole-3-carboxamide;
6-(3-((5-Chloro-2-methoxypyridine)-3-sulfonamido)-2,6-difluorophenyl)-N-ethyl-7-fluoro-1H-indazole-3-carboxamide;
6-[3-(5-Chloro-2-methoxypyridine-3-sulfonamido)-2,6-difluorophenyl]-7-fluoro-N-(1-methyl-6-oxopiperidin-3-yl)-1H-indazole-3-carboxamide;
N-(1-acetylpyrrolidin-3-yl)-6-[3-(5-chloro-2-methoxypyridine-3-sulfonamido)-2,6-difluorophenyl]-7-fluoro-1H-indazole-3-carboxamide;
6-[3-(5-Chloro-2-methoxypyridine-3-sulfonamido)-2,6-difluorophenyl]-7-fluoro-N-(2-hydroxypropyl)-1H-indazole-3-carboxamide;
6-[3-(5-Chloro-2-methoxypyridine-3-sulfonamido)-2,6-difluorophenyl]-7-fluoro-N-(1-hydroxypropan-2-yl)-1H-indazole-3-carboxamide;
6-[3-(5-Chloro-2-methoxypyridine-3-sulfonamido)-2,6-difluorophenyl]-7-fluoro-N-(2-oxopyrrolidin-3-yl)-1H-indazole-3-carboxamide;
6-[3-(5-Chloro-2-methoxypyridine-3-sulfonamido)-2,6-difluorophenyl]-7-fluoro-N-(oxan-3-yl)-1H-indazole-3-carboxamide;
6-[3-(5-Chloro-2-methoxypyridine-3-sulfonamido)-2,6-difluorophenyl]-7-fluoro-N-(sec-butyl)-1H-indazole-3-carboxamide;
6-[3-(5-Chloro-2-methoxypyridine-3-sulfonamido)-2,6-difluorophenyl]-7-fluoro-N-(pyrrolidin-3-yl)-1H-indazole-3-carboxamide;
6-[3-(5-Chloro-2-methoxypyridine-3-sulfonamido)-2,6-difluorophenyl]-7-fluoro-N-propyl-1H-indazole-3-carboxamide;
6-[3-(5-Chloro-2-methoxypyridine-3-sulfonamido)-2,6-difluorophenyl]-7-fluoro-N-(pentan-2-yl)-1H-indazole-3-carboxamide;
6-[3-(5-chloro-2-methoxypyridine-3-sulfonamido)-2,6-difluorophenyl]-7-fluoro-N-(4-hydroxybutan-2-yl)-1H-indazole-3-carboxamide;
6-[3-(1-Benzofuran-4-sulfonamido)-2,6-difluorophenyl]-7-fluoro-N-methyl-1H-indazole-3-carboxamide;
6-[3-(1H-1,3-Benzodiazole-4-sulfonamido)-2,6-difluorophenyl]-7-fluoro-N-methyl-1H-indazole-3-carboxamide;
6-[2,6-Difluoro-3-(3-methyl-1,3-benzodiazole-4-sulfonamido)phenyl]-7-fluoro-N-methyl-1H-indazole-3-carboxamide;
5-chloro-N-[2,4-difluoro-3-[7-fluoro-3-(1,3-oxazol-2-yl)-1H-indazol-6-yl]phenyl]-2-methoxypyridine-3-sulfonamide;
6-[2,6-difluoro-3-(5-fluoro-2-methylpyridine-3-sulfonamido)phenyl]-7-fluoro-N-methyl-1H-indazole-3-carboxamide;
6-[2,6-Difluoro-3-(5-fluoro-2-methylpyridine-3-sulfonamido)phenyl]-7-fluoro-N-methyl-1H-pyrazolo[4,3-c]pyridine-3-carboxamide;
6-[3-(5-Chloro-2-methoxypyridine-3-sulfonamido)-2,6-difluorophenyl]-7-fluoro-N-methyl-1H-pyrazolo[4,3-c]pyridine-3-carboxamide;
N-[2,4-difluoro-3-[7-fluoro-3-(hydrazinecarbonyl)-1H-indazol-6-yl]phenyl]-1-benzofuran-6-sulfonamide;
6-[2,6-Difluoro-3-(1-methyl-1,3-benzodiazole-4-sulfonamido)phenyl]-7-fluoro-N-methyl-1H-indazole-3-carboxamide;
6-[2,6-Difluoro-3-(5-fluoro-2-methoxypyridine-3-sulfonamido)phenyl]-7-fluoro-N-methyl-1H-pyrazolo[4,3-c]pyridine-3-carboxamide;
6-[3-(5-Cyano-2-methoxypyridine-3-sulfonamido)-2,6-difluorophenyl]-7-fluoro-N-methyl-1H-pyrazolo[4,3-c]pyridine-3-carboxamide;
6-[3-(5-Chloro-2-methylpyridine-3-sulfonamido)-2,6-difluorophenyl]-7-fluoro-N-methyl-1H-pyrazolo[4,3-c]pyridine-3-carboxamide;
6-[3-(cyclopentylmethanesulfonamido)-2,6-difluorophenyl]-7-fluoro-N-methyl-1H-indazole-3-carboxamide;
6-[2,6-difluoro-3-(oxane-4-sulfonamido)phenyl]-7-fluoro-N-methyl-1H-indazole-3-carboxamide;
6-[2,6-Difluoro-3-(6-fluoro-1-hydroxy-2,3-dihydro-1H-indene-4-sulfonamido)phenyl]-7-fluoro-N-methyl-1H-indazole-3-carboxamide;
6-[2,6-difluoro-3-(1-methylpiperidine-3-sulfonamido)phenyl]-7-fluoro-N-methyl-1H-indazole-3-carboxamide;
6-[2,6-difluoro-3-[(3-hydroxycyclopentyl)methanesulfonamido]phenyl]-7-fluoro-N-methyl-1H-indazole-3-carboxamide;
6-[3-(5-Chloro-2-methoxypyridine-3-sulfonamido)-2,6-difluorophenyl]-N-cyclopentyl-7-fluoro-1H-indazole-3-carboxamide;
6-[3-(5-Chloro-2-methoxypyridine-3-sulfonamido)-2,6-difluorophenyl]-7-fluoro-N-(3-hydroxycyclohexyl)-1H-indazole-3-carboxamide;
6-[3-(5-Chloro-2-methoxypyridine-3-sulfonamido)-2,6-difluorophenyl]-N-cyclopropyl-7-fluoro-1H-indazole-3-carboxamide;
6-[3-(5-Chloro-2-ethoxypyridine-3-sulfonamido)-2,6-difluorophenyl]-7-fluoro-N-(morpholin-4-ylmethyl)-1H-indazole-3-carboxamide;
6-[3-(5-chloro-2-methoxypyridine-3-sulfonamido)-2,6-difluorophenyl]-7-fluoro-N-[2-(pyrrolidin-1-yl)ethyl]-1H-indazole-3-carboxamide;
6-[3-(5-Chloro-2-methoxypyridine-3-sulfonamido)-2,6-difluorophenyl]-7-fluoro-N-(2-methanesulfonylethyl)-1H-indazole-3-carboxamide;
6-[3-(5-Chloro-2-methoxypyridine-3-sulfonamido)-2,6-difluorophenyl]-N-(cyclopropylmethyl)-7-fluoro-1H-indazole-3-carboxamide;
6-[3-(5-Chloro-2-methoxypyridine-3-sulfonamido)-2,6-difluorophenyl]-7-fluoro-N-[2-(morpholin-4-yl)ethyl]-1H-indazole-3-carboxamide;
6-[3-(5-Chloro-2-methoxypyridine-3-sulfonamido)-2,6-difluorophenyl]-7-fluoro-N-(2-hydroxycyclopentyl)-1H-indazole-3-carboxamide;
6-[3-(5-Chloro-2-methoxypyridine-3-sulfonamido)-2,6-difluorophenyl]-7-fluoro-N-(2-hydroxycyclohexyl)-1H-indazole-3-carboxamide;
6-[3-(5-Chloro-2-methoxypyridine-3-sulfonamido)-2,6-difluorophenyl]-7-fluoro-N-(2-methylbut-3-yn-2-yl)-1H-indazole-3-carboxamide;
6-[3-(5-Chloro-2-methoxypyridine-3-sulfonamido)-2,6-difluorophenyl]-7-fluoro-N-(2,2,2-trifluoroethyl)-1H-indazole-3-carboxamide;
6-[3-(5-Chloro-2-methoxypyridine-3-sulfonamido)-2,6-difluorophenyl]-N-[cyano(cyclopropyl)methyl]-7-fluoro-1H-indazole-3-carboxamide;
6-[3-(5-Chloro-2-methoxypyridine-3-sulfonamido)-2,6-difluorophenyl]-7-fluoro-N-(prop-2-yn-1-yl)-1H-indazole-3-carboxamide;
6-[3-(5-Chloro-2-methoxypyridine-3-sulfonamido)-2,6-difluorophenyl]-7-fluoro-N-(piperidin-3-ylmethyl)-1H-indazole-3-carboxamide;
N-(2-Aminocyclohexyl)-6-[3-(5-chloro-2-methoxypyridine-3-sulfonamido)-2,6-difluorophenyl]-7-fluoro-1H-indazole-3-carboxamide;
N-(1-Aminopropan-2-yl)-6-[3-(5-chloro-2-methoxypyridine-3-sulfonamido)-2,6-difluorophenyl]-7-fluoro-1H-indazole-3-carboxamide;
6-[3-(5-Chloro-2-methoxypyridine-3-sulfonamido)-2,6-difluorophenyl]-7-fluoro-N-(4-oxocyclohexyl)-1H-indazole-3-carboxamide;
6-[3-(5-Chloro-2-methoxypyridine-3-sulfonamido)-2,6-difluorophenyl]-7-fluoro-N-(3-hydroxycyclopentyl)-1H-indazole-3-carboxamide;
N-(3-Aminocyclohexyl)-6-[3-(5-chloro-2-methoxypyridine-3-sulfonamido)-2,6-difluorophenyl]-7-fluoro-1H-indazole-3-carboxamide;
6-[3-(5-Chloro-2-methoxypyridine-3-sulfonamido)-2,6-difluorophenyl]-7-fluoro-N-[2-(2-oxopyrrolidin-1-yl)ethyl]-1H-indazole-3-carboxamide;
6-[3-(5-Chloro-2-methoxypyridine-3-sulfonamido)-2,6-difluorophenyl]-7-fluoro-N-[(1-methylpyrrolidin-3-yl)methyl]-1H-indazole-3-carboxamide;
6-[3-(1,3-dihydro-2-benzofuran-4-sulfonamido)-2,6-difluorophenyl]-7-fluoro-N-methyl-1H-indazole-3-carboxamide;
6-[3-(2,3-dihydro-1-benzofuran-6-sulfonamido)-2,6-difluorophenyl]-7-fluoro-N-methyl-1H-indazole-3-carboxamide;
6-[3-(2,3-Dihydro-1-benzofuran-6-sulfonamido)-2,6-difluorophenyl]-7-fluoro-N-methyl-1-[[2-(trimethylsilyl)ethoxy]methyl]indazole-3-carboxamide;
6-[3-(6-cyano-1-hydroxy-2,3-dihydro-1H-indene-4-sulfonamido)-2,6-difluorophenyl]-7-fluoro-N-methyl-1H-indazole-3-carboxamide;
6-[2,6-difluoro-3-(6-fluoro-1-hydroxy-2,3-dihydro-1H-indene-4-sulfonamido)phenyl]-7-fluoro-N-methyl-1H-pyrazolo[4,3-c]pyridine-3-carboxamide;
or a pharmaceutically acceptable salt thereof.

88. A pharmaceutical composition comprising a compound of claim 1 and a pharmaceutically acceptable carrier.

89. A method of treating cancer in a subject, comprising administering a therapeutically effective amount of a compound of claim 1 to a subject in need thereof to treat the cancer.

90. The method of claim 89, wherein the cancer is colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, sweat gland carcinoma, sebaceous gland carcinoma, thyroid cancer, kidney cancer, uterus cancer, esophagus cancer, liver cancer, head cancer, neck cancer, throat cancer, mouth cancer, bone cancer, chest cancer, lymph node cancer, eye cancer, mesothelioma, an acoustic neuroma, oligodendroglioma, meningioma, neuroblastoma, retinoblastoma, leukemia, or lymphoma.

91. (canceled)

92. A method of treating a neurodegenerative disease in a subject, comprising administering a therapeutically effective amount of a compound of claim 1 to a subject in need thereof to treat the neurodegenerative disease.

93. The method of claim 92, wherein the neurodegenerative disease is Alzheimer's disease, Parkinson's Disease, Huntington's Disease, amyotrophic lateral sclerosis, or spinocerebellar ataxia.

94. A method of treating doxorubicin-induced cardiotoxicity in a subject, comprising administering a therapeutically effective amount of a compound of claim 1 to a subject in need thereof suffering from doxorubicin-induced cardiotoxicity, to thereby treat the doxorubicin-induced cardiotoxicity.

95.-96. (canceled)
